(19) United States
(12) Patent Application Publication
(10) Pub. No.: US 2026/0327658 A1
(43) Pub. Date: Oct. 22, 2026

(54) TANDEM FUSOGENS AND RELATED LIPID PARTICLES

(71) Applicant: Sana Biotechnology, Inc., Seattle, WA (US)

(72) Inventors: Patricia Ann CRUITE, Seattle, WA (US); Matteo STOPPATO, Stoneham, MA (US); Jagesh Vijaykumar SHAH, Lexington, MA (US); Luca BIASCO, Boston, MA (US)

(73) Assignee: Sana Biotechnology, Inc., Seattle, WA (US)

(21) Appl. No.: 19/485,225

(22) PCT Filed: May 22, 2024
(86) PCT No.: PCT/US2024/030619

§ 371 Date: Nov. 17, 2025

Related U.S. Application Data
(60) Provisional application No. 63/468,525, filed on May 23, 2023.
(60) Provisional application No. 63/522,722, filed on Jun. 22, 2023.
(60) Provisional application No. 63/539,547, filed on Sep. 20, 2023.
(60) Provisional application No. 63/592,151, filed on Oct. 20, 2023.

(51) Int. Cl. C12N 15/88; A61K 31/395; A61K 38/19; C07K 14/005; C07K 16/28; C12N 15/86

(52) U.S. Cl. CPC C12N 15/88; A61K 31/395; A61K 38/193; C07K 14/005; C07K 16/2803; C07K 16/2815; C07K 16/2896; C12N 15/86; C07K 2317/569; C07K 2317/622; C07K 2319/00; C12N 2740/15043; C12N 2740/15051; C12N 2760/18222; C12N 2760/18223

(57) ABSTRACT

Provided herein are lipid particles containing at least two attachment proteins derived from a paramyxovirus envelope attachment protein and at least one paramyxovirus fusion (F) protein. Also provided are lipid particles are viral vectors, such as lentiviral vectors or lentiviral-like particles. Also provided are producer cells and compositions containing such lipid particles and methods of making and using the lipid particles.

### CROSS-REFERENCE TO RELATED APPLICATIONS

This application claims priority to U.S. Provisional Patent Application No. 63/468,525 filed on May 23, 2023, entitled “Tandem Fusogens and Related Lipid Particles”, U.S. Provisional Patent Application No. 63/522,722 filed on Jun. 22, 2023, entitled “Tandem Fusogens and Related Lipid Particles”, U.S. Provisional Patent Application No. 63/539,547 filed on Sep. 20, 2023, entitled “Tandem Fusogens and Related Lipid Particles”, U.S. Provisional Patent Application No. 63/592,151 filed on Oct. 20, 2023, entitled “Tandem Fusogens and Related Lipid Particles”, each of which are hereby incorporated by reference in its entirety.

### REFERENCE TO AN ELECTRONIC SEQUENCE LISTING

The present application is being filed along with a Sequence Listing in electronic format. The Sequence Listing is provided as a file entitled 18615_2009440.XML created May 22, 2024, 2024 which is 1,090,341 bytes in size. The information in the electronic format of the Sequence Listing is incorporated by reference in its entirety.

### FIELD

The present disclosure relates to lipid particles containing at least one attachment protein derived from a paramyxovirus envelope attachment protein and at least one paramyxovirus fusion (F) protein. In some embodiments, the attachment protein is retargeted, such as with two or more targeting moieties. In some embodiments, the lipid particles are viral vectors, such as lentiviral vectors or lentiviral-like particles. Also provided are producer cells and compositions containing such lipid particles and methods of making and using the lipid particles.

### BACKGROUND

Lipid particles, including viral-based particles like virus-like particles and viral vectors such as lentiviral particles, are commonly used for delivery of exogenous agents to cells. For various particles, such as lentiviral vector particles, the host range can be altered by pseudotyping with at least one retargeted attachment protein that is or comprises two or more targeting moieties. The efficient preparation and production of particles with certain retargeted pseudotyped envelope proteins to produce a higher titer and with efficient transduction efficiency of target cells are needed. The provided disclosure addresses this need.

### SUMMARY

Provided herein is a lipid particle, comprising: (a) a retargeted attachment protein comprising a paramyxovirus envelope attachment protein linked to (i) a first targeting moiety directed to a first target molecule expressed on the surface of a target cell, and (ii) a second targeting moiety directed to a second target molecule expressed on the surface of a target cell; and (b) at least one paramyxovirus fusion (F) protein; and wherein the protein in (a), and (b) are exposed on the outside of the lipid bilayer.

In some of any of the provided embodiments, the paramyxovirus attachment protein is a variant paramyxovirus envelope attachment protein comprising one or more mutations to reduce native tropism relative to the wild-type paramyxovirus envelope attachment protein not comprising the one or more mutations. In some of any of the provided embodiments, the paramyxovirus envelope attachment protein is a first paramyxovirus envelope attachment protein and the lipid particle further comprises a second paramyxovirus envelope attachment protein, wherein the second paramyxovirus envelope attachment protein is a variant paramyxovirus envelope attachment protein comprising one or more mutations to reduce native tropism relative to the wild-type paramyxovirus envelope attachment protein not comprising the one or more mutations, wherein the second paramyxovirus envelope attachment protein is exposed on the outside of the lipid bilayer. In some of any of the provided embodiments, the targeting one or both of the first target molecule and the second target molecule does not activate or inhibit, induce a phenotype change (for example maturation and/or differentiation), induce proliferation, and/or induce apoptosis of said target cell.

Provided herein is a lipid particle, comprising: (a) a retargeted attachment protein comprising a first paramyxovirus envelope attachment protein operably linked to (i) a first targeting moiety directed to a first target molecule expressed on the surface of a target cell, and (ii) a second targeting moiety directed to a second target molecule expressed on the surface of a target cell, (b) a second paramyxovirus envelope attachment protein that is a variant paramyxovirus envelope attachment protein comprising one or more mutations to reduce the native tropism relative to the wild-type paramyxovirus envelope attachment protein not comprising the one or more mutations; and (c) at least one paramyxovirus fusion (F) protein; and wherein the protein in (a), (b) and (c) are exposed on the outside of the lipid bilayer. Provided herein is a lipid particle, comprising: (a) a retargeted attachment protein comprising a first paramyxovirus envelope attachment protein operably linked to (i) a first targeting moiety directed to a first target molecule expressed on the surface of a target cell, and (ii) a second targeting moiety directed to a second target molecule expressed on the surface of a target cell, (b) a second paramyxovirus envelope attachment protein that is a variant paramyxovirus envelope attachment protein comprising one or more amino acid substitutions to reduce the native tropism relative to the wild-type paramyxovirus envelope attachment protein not comprising the one or more mutations; and (c) at least one paramyxovirus fusion (F) protein; and wherein the protein in (a), (b) and (c) are exposed on the outside of the lipid bilayer.

In some of any of the provided embodiments, the first paramyxovirus envelope attachment protein is a variant paramyxovirus envelope attachment protein. In some of any of the provided embodiments, the variant paramyxovirus envelope attachment protein comprises one or more mutations that reduces native tropism relative to the wild-type paramyxovirus envelope attachment protein not comprising the one or more mutations. In some of any of the provided embodiments, the first and second paramyxovirus envelope attachment protein are the same. In some of any of the provided embodiments, the second paramyxovirus envelope attachment protein is not linked or fused to a non-viral heterologous moiety that is a cell-specific targeting domain or functional domain. In some of any of the provided embodiments, the first and second targeting moiety are each not selected from the group consisting of a cytokine, growth factor, hormone, neurotransmitter, apoptosis ligand and their combinations.

In some of any of the provided embodiments, targeting one or both of the first target molecule and the second target molecule does not modulate or induce a signal in the target cell. In some of any of the provided embodiments, the first and second targeting moieties each bind to a cell surface molecule present on a target cell. In some of any of the provided embodiments, the first targeting moiety binds to a cell surface molecule present on a first target cell, and the second targeting moiety binds a surface molecule present on a second target cell. In some of any of the provided embodiments, the first and second target molecule are different target molecules. In some of any of the provided embodiments, the first and second target molecule are the same target molecule. In some of any of the provided embodiments, the first and second targeting moiety bind distinct epitopes of the same target molecule.

In some of any of the provided embodiments, the cell surface molecule is a protein, glycan, or lipid. In some of any of the provided embodiments, the target cell is selected from the group consisting of tumor-infiltrating lymphocytes, T cells, neoplastic or tumor cells, virus-infected cells, stem cells, central nervous system (CNS) cells, hematopoietic stem cells (HSCs), and liver cells. In some of any of the provided embodiments, the target cells is selected from the group consisting of a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+B cell, a CD19+B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell. In some of any of the provided embodiments, the target cell is a hepatocyte. In some of any of the provided embodiments, the cell surface molecule is selected from the group consisting of CD34, CD117, and CD133. In some of any of the provided embodiments, the target cell is a T cell. In some of any of the provided embodiments, the cell surface molecule is selected from the group consisting of CD3, CD4, CD7 CD8, ASCT2, CD105, CD110, CD117, CD133, CD146, CD164, CD34, CD46, CD49f, CD90, EPCR, and ITGA3.

In some of any of the provided embodiments, the second paramyxovirus envelope attachment protein that is a variant paramyxovirus envelope attachment protein comprises one or more amino acid substitutions corresponding to amino acid substitutions selected from the group consisting of E501A, W504A, Q530A and E533A with reference to numbering set forth in SEQ ID NO:1.

Provided herein is a lipid particle, comprising: (a) a first retargeted attachment protein comprising (i) a first paramyxovirus envelope attachment protein; and (ii) a first targeting moiety directed to CD133; and a second targeting moiety directed to CD133; and (b) at least one paramyxovirus fusion (F) protein; and the protein in (a), and (b) are exposed on the outside of the lipid bilayer.

In some of any of the provided embodiments, (i) the first targeting moiety is an scFv and comprises an amino acid sequence selected from the group consisting of SEQ ID NOs: 516, 525, 534, 543, and 552, or an amino acid sequence having at least 90%, 91%, 92%, 93%, 94%, 95%, 96%, 97%, 98%, or 99% sequence identity thereto; and/or (ii) the second targeting moiety is an scFv and comprises an amino acid sequence selected from the group consisting of SEQ ID NOs: 516, 525, 534, 543, and 552, or an amino acid sequence having at least 90%, 91%, 92%, 93%, 94%, 95%, 96%, 97%, 98%, or 99% sequence identity thereto.

In some of any of the provided embodiments, (i) the first targeting moiety comprises: (a) a CD133 binding agent comprising a CDR-H1, a CDR-H2, and a CDR-H3 comprising the amino acid sequences of SEQ ID NOs: 536, 537, and 538, respectively, and a CDR-L1, a CDR-L2, and a CDR-L3 comprising the amino acid sequences of SEQ ID NOs: 540, 541, and 542, respectively; (b) a CD133 binding agent comprising a CDR-H1, a CDR-H2, and a CDR-H3 comprising the amino acid sequences of SEQ ID NOs: 545, 546, and 547, respectively, and a CDR-L1, a CDR-L2, and a CDR-L3 comprising the amino acid sequences of SEQ ID NOs: 549, 550, and 551, respectively; (c) a CD133 binding agent comprising a CDR-H1, a CDR-H2, and a CDR-H3 comprising the amino acid sequences of SEQ ID NOs: 518, 519, and 520, respectively, and a CDR-L1, a CDR-L2, and a CDR-L3 comprising the amino acid sequences of SEQ ID NOs: 522, 523, and 524, respectively; (d) a CD133 binding agent comprising a CDR-H1, a CDR-H2, and a CDR-H3 comprising the amino acid sequences of SEQ ID NOs: 527, 528, and 529, respectively, and a CDR-L1, a CDR-L2, and a CDR-L3 comprising the amino acid sequences of SEQ ID NOs: 531, 532, and 533, respectively; or (e) a CD133 binding agent comprising a CDR-H1, a CDR-H2, and a CDR-H3 comprising the amino acid sequences of SEQ ID NOs: 554, 555, and 556, respectively, and a CDR-L1, a CDR-L2, and a CDR-L3 comprising the amino acid sequences of SEQ ID NOs: 558, 559, and 560, respectively; and/or (ii) the second targeting moiety comprises: (a) a CD133 binding agent comprising a CDR-H1, a CDR-H2, and a CDR-H3 comprising the amino acid sequences of SEQ ID NOs: 536, 537, and 538, respectively, and a CDR-L1, a CDR-L2, and a CDR-L3 comprising the amino acid sequences of SEQ ID NOs: 540, 541, and 542, respectively; (b) a CD133 binding agent comprising a CDR-H1, a CDR-H2, and a CDR-H3 comprising the amino acid sequences of SEQ ID NOs: 545, 546, and 547, respectively, and a CDR-L1, a CDR-L2, and a CDR-L3 comprising the amino acid sequences of SEQ ID NOs: 549, 550, and 551, respectively; (c) a CD133 binding agent comprising a CDR-H1, a CDR-H2, and a CDR-H3 comprising the amino acid sequences of SEQ ID NOs: 518, 519, and 520, respectively, and a CDR-L1, a CDR-L2, and a CDR-L3 comprising the amino acid sequences of SEQ ID NOs: 522, 523, and 524, respectively; (d) a CD133 binding agent comprising a CDR-H1, a CDR-H2, and a CDR-H3 comprising the amino acid sequences of SEQ ID NOs: 527, 528, and 529, respectively, and a CDR-L1, a CDR-L2, and a CDR-L3 comprising the amino acid sequences of SEQ ID NOs: 531, 532, and 533, respectively; or (e) a CD133 binding agent comprising a CDR-H1, a CDR-H2, and a CDR-H3 comprising the amino acid sequences of SEQ ID NOs: 554, 555, and 556, respectively, and a CDR-L1, a CDR-L2, and a CDR-L3 comprising the amino acid sequences of SEQ ID NOs: 558, 559, and 560, respectively.

In some of any of the provided embodiments, (i) the first targeting moiety comprises: (a) a CD133 binding agent comprising a CDR-H1, a CDR-H2, and a CDR-H3 comprising the amino acid sequences of SEQ ID NOs: 289, 565, and 538, respectively, and a CDR-L1, a CDR-L2, and a CDR-L3 comprising the amino acid sequences of SEQ ID NOs: 540, 541, and 542, respectively; (b) a CD133 binding agent comprising a CDR-H1, a CDR-H2, and a CDR-H3 comprising the amino acid sequences of SEQ ID NOs: 566, 567, and 547, respectively, and a CDR-L1, a CDR-L2, and a CDR-L3 comprising the amino acid sequences of SEQ ID NOs: 549, 550, and 551, respectively; (c) a CD133 binding agent comprising a CDR-H1, a CDR-H2, and a CDR-H3 comprising the amino acid sequences of SEQ ID NOs: 568, 569, and 520, respectively, and a CDR-L1, a CDR-L2, and a CDR-L3 comprising the amino acid sequences of SEQ ID NOs: 522, 523, and 524, respectively; (d) a CD133 binding agent comprising a CDR-H1, a CDR-H2, and a CDR-H3 comprising the amino acid sequences of SEQ ID NOs: 570, 571, and 529, respectively, and a CDR-L1, a CDR-L2, and a CDR-L3 comprising the amino acid sequences of SEQ ID NOs: 531, 532, and 533, respectively; or (e) a CD133 binding agent comprising a CDR-H1, a CDR-H2, and a CDR-H3 comprising the amino acid sequences of SEQ ID NOs: 572, 573, and 556, respectively, and a CDR-L1, a CDR-L2, and a CDR-L3 comprising the amino acid sequences of SEQ ID NOs: 558, 559, and 560, respectively; and/or (ii) the second targeting moiety comprises: (a) a CD133 binding agent comprising a CDR-H1, a CDR-H2, and a CDR-H3 comprising the amino acid sequences of SEQ ID NOs: 289, 565, and 538, respectively, and a CDR-L1, a CDR-L2, and a CDR-L3 comprising the amino acid sequences of SEQ ID NOs: 540, 541, and 542, respectively; (b) a CD133 binding agent comprising a CDR-H1, a CDR-H2, and a CDR-H3 comprising the amino acid sequences of SEQ ID NOs: 566, 567, and 547, respectively, and a CDR-L1, a CDR-L2, and a CDR-L3 comprising the amino acid sequences of SEQ ID NOs: 549, 550, and 551, respectively; (c) a CD133 binding agent comprising a CDR-H1, a CDR-H2, and a CDR-H3 comprising the amino acid sequences of SEQ ID NOs: 568, 569, and 520, respectively, and a CDR-L1, a CDR-L2, and a CDR-L3 comprising the amino acid sequences of SEQ ID NOs: 522, 523, and 524, respectively; (d) a CD133 binding agent comprising a CDR-H1, a CDR-H2, and a CDR-H3 comprising the amino acid sequences of SEQ ID NOs: 570, 571, and 529, respectively, and a CDR-L1, a CDR-L2, and a CDR-L3 comprising the amino acid sequences of SEQ ID NOs: 531, 532, and 533, respectively; or (e) a CD133 binding agent comprising a CDR-H1, a CDR-H2, and a CDR-H3 comprising the amino acid sequences of SEQ ID NOs: 572, 573, and 556, respectively, and a CDR-L1, a CDR-L2, and a CDR-L3 comprising the amino acid sequences of SEQ ID NOs: 558, 559, and 560, respectively.

In some of any of the provided embodiments, (i) the first targeting moiety comprises: (a) a CD133 binding agent comprising a heavy chain variable (VH) region comprising the amino acid sequence of SEQ ID NO: 535, or an amino acid sequence having at least 90%, 91%, 92%, 93%, 94%, 95%, 96%, 97%, 98%, or 99% sequence identity thereto; and a light chain variable (VL) region comprising the amino acid sequence of SEQ ID NO: 539, or an amino acid sequence having at least 90%, 91%, 92%, 93%, 94%, 95%, 96%, 97%, 98%, or 99% sequence identity thereto; (b) a CD133 binding agent comprising a VH region comprising the amino acid sequence of SEQ ID NO: 544, or an amino acid sequence having at least 90%, 91%, 92%, 93%, 94%, 95%, 96%, 97%, 98%, or 99% sequence identity thereto; and a VL region comprising the amino acid sequence of SEQ ID NO: 548, or an amino acid sequence having at least 90%, 91%, 92%, 93%, 94%, 95%, 96%, 97%, 98%, or 99% sequence identity thereto; (c) a CD133 binding agent comprising a VH region comprising the amino acid sequence of SEQ ID NO: 517, or an amino acid sequence having at least 90%, 91%, 92%, 93%, 94%, 95%, 96%, 97%, 98%, or 99% sequence identity thereto; and a VL region comprising the amino acid sequence of SEQ ID NO: 521, or an amino acid sequence having at least 90%, 91%, 92%, 93%, 94%, 95%, 96%, 97%, 98%, or 99% sequence identity thereto; (d) a CD133 binding agent comprising a VH region comprising the amino acid sequence of SEQ ID NO: 526, or an amino acid sequence having at least 90%, 91%, 92%, 93%, 94%, 95%, 96%, 97%, 98%, or 99% sequence identity thereto; and a VL region comprising the amino acid sequence of SEQ ID NO: 530, or an amino acid sequence having at least 90%, 91%, 92%, 93%, 94%, 95%, 96%, 97%, 98%, or 99% sequence identity thereto; or (e) a CD133 binding agent comprising a VH region comprising the amino acid sequence of SEQ ID NO: 553, or an amino acid sequence having at least 90%, 91%, 92%, 93%, 94%, 95%, 96%, 97%, 98%, or 99% sequence identity thereto; and a VL region comprising the amino acid sequence of SEQ ID NO: 557, or an amino acid sequence having at least 90%, 91%, 92%, 93%, 94%, 95%, 96%, 97%, 98%, or 99% sequence identity thereto; and/or (ii) the second targeting moiety comprises: (a) a CD133 binding agent comprising a heavy chain variable (VH) region comprising the amino acid sequence of SEQ ID NO: 535, or an amino acid sequence having at least 90%, 91%, 92%, 93%, 94%, 95%, 96%, 97%, 98%, or 99% sequence identity thereto; and a light chain variable (VL) region comprising the amino acid sequence of SEQ ID NO: 539, or an amino acid sequence having at least 90%, 91%, 92%, 93%, 94%, 95%, 96%, 97%, 98%, or 99% sequence identity thereto; (b) a CD133 binding agent comprising a VH region comprising the amino acid sequence of SEQ ID NO: 544, or an amino acid sequence having at least 90%, 91%, 92%, 93%, 94%, 95%, 96%, 97%, 98%, or 99% sequence identity thereto; and a VL region comprising the amino acid sequence of SEQ ID NO: 548, or an amino acid sequence having at least 90%, 91%, 92%, 93%, 94%, 95%, 96%, 97%, 98%, or 99% sequence identity thereto; (c) a CD133 binding agent comprising a VH region comprising the amino acid sequence of SEQ ID NO: 517, or an amino acid sequence having at least 90%, 91%, 92%, 93%, 94%, 95%, 96%, 97%, 98%, or 99% sequence identity thereto; and a VL region comprising the amino acid sequence of SEQ ID NO: 521, or an amino acid sequence having at least 90%, 91%, 92%, 93%, 94%, 95%, 96%, 97%, 98%, or 99% sequence identity thereto; (d) a CD133 binding agent comprising a VH region comprising the amino acid sequence of SEQ ID NO: 526, or an amino acid sequence having at least 90%, 91%, 92%, 93%, 94%, 95%, 96%, 97%, 98%, or 99% sequence identity thereto; and a VL region comprising the amino acid sequence of SEQ ID NO: 530, or an amino acid sequence having at least 90%, 91%, 92%, 93%, 94%, 95%, 96%, 97%, 98%, or 99% sequence identity thereto; or (e) a CD133 binding agent comprising a VH region comprising the amino acid sequence of SEQ ID NO: 553, or an amino acid sequence having at least 90%, 91%, 92%, 93%, 94%, 95%, 96%, 97%, 98%, or 99% sequence identity thereto; and a VL region comprising the amino acid sequence of SEQ ID NO: 557, or an amino acid sequence having at least 90%, 91%, 92%, 93%, 94%, 95%, 96%, 97%, 98%, or 99% sequence identity thereto.

In some of any of the provided embodiments, (a) the first targeting moiety comprises a CD133 binding agent comprising a VH region comprising the amino acid sequence of SEQ ID NO: 517, or an amino acid sequence having at least 90%, 91%, 92%, 93%, 94%, 95%, 96%, 97%, 98%, or 99% sequence identity thereto; and (b) the second targeting moiety comprises a CD133 binding agent comprising a VH region comprising the amino acid sequence of SEQ ID NO: 526, or an amino acid sequence having at least 90%, 91%, 92%, 93%, 94%, 95%, 96%, 97%, 98%, or 99% sequence identity thereto. In some of any of the provided embodiments, (a) the first targeting moiety comprises a CD133 binding agent comprising a VH region comprising the amino acid sequence of SEQ ID NO: 526, or an amino acid sequence having at least 90%, 91%, 92%, 93%, 94%, 95%, 96%, 97%, 98%, or 99% sequence identity thereto; and (b) the second targeting moiety comprises a CD133 binding agent comprising a VH region comprising the amino acid sequence of SEQ ID NO: 517, or an amino acid sequence having at least 90%, 91%, 92%, 93%, 94%, 95%, 96%, 97%, 98%, or 99% sequence identity thereto. In some of any of the provided embodiments, wherein the first and second targeting moiety bind to distinct epitopes on CD133.

In some of any of the provided embodiments, the lipid particle further comprises a second paramyxovirus envelope attachment protein that is a variant paramyxovirus envelope attachment protein comprising one or more amino acid substitutions to reduce the native tropism relative to the wild-type paramyxovirus envelope attachment protein not comprising the one or more amino acid substitutions. In some of any of the provided embodiments, the second paramyxovirus envelope attachment protein that is a variant paramyxovirus envelope attachment protein comprises one or more amino acid substitutions corresponding to amino acid substitutions selected from the group consisting of E501A, W504A, Q530A and E533A with reference to numbering set forth in SEQ ID NO:1.

Provided herein is a lipid particle, comprising: (a) a first retargeted attachment protein comprising (i) a first paramyxovirus envelope attachment protein; and (ii) a first targeting moiety directed to CD117; and a second targeting moiety directed to CD117; and (b) at least one paramyxovirus fusion (F) protein; and the protein in (a), (b) are exposed on the outside of the lipid bilayer.

In some of any of the provided embodiments, (a) the first targeting moiety comprises a CD117 binding agent comprising a VHH single domain antibody comprising a CDR-H1, a CDR-H2, and a CDR-H3 contained within an amino acid sequence selected from the group consisting of SEQ ID NOs: 512-515; and/or wherein (b) the second targeting moiety comprises a CD117 binding agent comprising a VHH single domain antibody comprising a CDR-H1, a CDR-H2, and a CDR-H3 contained within an amino acid sequence selected from the group consisting of SEQ ID NOs: 512-515. In some of any of the provided embodiments, (a) the first targeting moiety comprising a VHH single domain antibody comprising an amino acid sequence selected from the group consisting of SEQ ID NOs: 512-515, or an amino acid sequence having at least 90%, 91%, 92%, 93%, 94%, 95%, 96%, 97%, 98%, or 99% sequence identity thereto; and/or (b)) the second targeting moiety comprising a VHH single domain antibody comprising an amino acid sequence selected from the group consisting of SEQ ID NOs: 512-515, or an amino acid sequence having at least 90%, 91%, 92%, 93%, 94%, 95%, 96%, 97%, 98%, or 99% sequence identity thereto. In some of any of the provided embodiments, (a) the first targeting moiety comprises a VHH comprising the amino acid sequence set forth in any one of SEQ ID NOS: 512-515; and/or (b) the second targeting moiety comprises a VHH comprising the amino acid sequence set forth in any one of SEQ ID NOS: 512-515. In some of any of the provided embodiments, the first and second targeting moiety bind to distinct epitopes on CD117.

In some of any of the provided embodiments, the lipid particle further comprises a second paramyxovirus envelope attachment protein that is a variant paramyxovirus envelope attachment protein comprising one or more amino acid substitutions to reduce the native tropism relative to the wild-type paramyxovirus envelope attachment protein not comprising the one or more amino acid substitutions. In some of any of the provided embodiments, the second paramyxovirus envelope attachment protein that is a variant paramyxovirus envelope attachment protein comprises one or more amino acid substitutions corresponding to amino acid substitutions selected from the group consisting of E501A, W504A, Q530A and E533A with reference to numbering set forth in SEQ ID NO:1.

Provided herein is a lipid particle, comprising: (a) a first retargeted attachment protein comprising (i) a first paramyxovirus envelope attachment protein; and (ii) a first targeting moiety directed to CD8; and a second targeting moiety directed to CD8; and (b) at least one paramyxovirus fusion (F) protein; and the protein in (a), and (b) are exposed on the outside of the lipid bilayer.

In some of any of the provided embodiments, (i) the first targeting moiety is an scFv and comprises an amino acid sequence selected from the group consisting of SEQ ID NOs: 489, 496, 501, or 508 or an amino acid sequence having at least 90%, 91%, 92%, 93%, 94%, 95%, 96%, 97%, 98%, or 99% sequence identity thereto; and/or (ii) the second targeting moiety is an scFv and comprises an amino acid sequence selected from the group consisting of SEQ ID NOs: 489, 496, 501, or 508, or an amino acid sequence having at least 90%, 91%, 92%, 93%, 94%, 95%, 96%, 97%, 98%, or 99% sequence identity thereto. In some of any of the provided embodiments, (a) the first targeting moiety comprises a CD8 binding agent comprising a VHH single domain antibody comprising a CDR-H1, a CDR-H2, and a CDR-H3 contained within an amino acid sequence of SEQ ID NO: 377; and/or wherein (b) the second targeting moiety comprises a CD8 binding agent comprising a VHH single domain antibody comprising a CDR-H1, a CDR-H2, and a CDR-H3 contained within an amino acid sequence of SEQ ID NO: 377. In some of any of the provided embodiments, (a) the first targeting moiety comprises a VHH comprising the amino acid sequence set forth in SEQ ID NOS: 377; and/or (b) the second targeting moiety comprises a VHH comprising the amino acid sequence set forth in SEQ ID NO: 377. In some of any of the provided embodiments, the first targeting moiety comprises a VHH comprising the amino acid sequence set forth in SEQ ID NO: 377 or an amino acid sequence having at least 90%, 91%, 92%, 93%, 94%, 95%, 96%, 97%, 98%, or 99% sequence identity thereto; and/or the second targeting moiety is an scFv and comprises an amino acid sequence selected from the group consisting of SEQ ID NO: 501 or an amino acid sequence having at least 90%, 91%, 92%, 93%, 94%, 95%, 96%, 97%, 98%, or 99% sequence identity thereto. In some of any of the provided embodiments, the first targeting moiety is an scFv and comprises an amino acid sequence selected from the group consisting of SEQ ID NO: 501 or an amino acid sequence having at least 90%, 91%, 92%, 93%, 94%, 95%, 96%, 97%, 98%, or 99% sequence identity thereto; and the second targeting moiety comprises a VHH comprising the amino acid sequence set forth in SEQ ID NO: 377 or an amino acid sequence having at least 90%, 91%, 92%, 93%, 94%, 95%, 96%, 97%, 98%, or 99% sequence identity thereto.

In some of any of the provided embodiments, the first and second targeting moiety bind to distinct epitopes on CD8.

In some of any of the provided embodiments, the lipid particle further comprising a second paramyxovirus envelope attachment protein that is a variant paramyxovirus envelope attachment protein comprising one or more mutations that reduces the native tropism relative to the wild-type paramyxovirus envelope attachment protein not comprising the one or more mutations. In some of any of the provided embodiments, the distinct epitopes are non-overlapping. In some of any of the provided embodiments, the first and second targeting moiety bind to the distinct epitopes in a non-competitive manner.

In some of any of the provided embodiments, each of the first targeting moiety and the second targeting moiety are independently selected from the group consisting of an antibody or antigen-binding fragment, a DARPin, an Aptamer, an Affimer, an Affibody, a Knottin, an Avimer, a Monobody, an Anticalin, a Fynomer, and a targeting peptide. In some of any of the provided embodiments, the first targeting moiety and the second targeting moiety are independently selected from the group consisting of a single domain antibody or a single chain variable fragment (scFv).

Provided herein is a lipid particle comprising a) a retargeted attachment protein comprising (i) a first paramyxovirus envelope attachment protein; and (ii) a first and second targeting moiety directed to CD133, (b) a second paramyxovirus envelope attachment protein that is a variant paramyxovirus envelope attachment protein comprising one or more mutations that reduces the native tropism relative to the wild-type paramyxovirus envelope attachment protein not comprising the one or more mutations; and (c) at least one paramyxovirus fusion (F) protein; and wherein the protein in (a), (b) and (c) are exposed on the outside of the lipid bilayer.

Provided herein is a lipid particle comprising (a) a retargeted attachment protein comprising (i) a first paramyxovirus envelope attachment protein; and (ii) a first and second targeting moiety directed to CD117, (b) a second paramyxovirus envelope attachment protein that is a variant paramyxovirus envelope attachment protein comprising one or more mutations that reduces the native tropism relative to the wild-type paramyxovirus envelope attachment protein not comprising the one or more mutations; and (c) at least one paramyxovirus fusion (F) protein; and wherein the protein in (a), (b) and (c) are exposed on the outside of the lipid bilayer.

Provided herein is a lipid particle comprising (a) a retargeted attachment protein comprising (i) a first paramyxovirus envelope attachment protein; and (ii) a first and second targeting moiety directed to CD8, (b) a second paramyxovirus envelope attachment protein that is a variant paramyxovirus envelope attachment protein comprising one or more mutations that reduces the native tropism relative to the wild-type paramyxovirus envelope attachment protein not comprising the one or more mutations; and (c) at least one paramyxovirus fusion (F) protein; and wherein the protein in (a), (b) and (c) are exposed on the outside of the lipid bilayer.

In some of any of the provided embodiments, the first paramyxovirus envelope attachment protein is a variant paramyxovirus envelope attachment protein. In some of any of the provided embodiments, the variant paramyxovirus envelope attachment protein comprises one or more mutations that reduces native tropism relative to the wild-type paramyxovirus envelope attachment protein not comprising the one or more mutations. In some of any of the provided embodiments, the second paramyxovirus envelope attachment protein is a variant paramyxovirus envelope attachment protein. In some of any of the provided embodiments, the variant paramyxovirus envelope attachment protein comprises one or more mutations that reduces native tropism relative to the wild-type paramyxovirus envelope attachment protein not comprising the one or more mutations. In some of any of the provided embodiments, the first targeting moiety is selected from the group consisting of a single domain antibody or a single chain variable fragment (scFv). In some of any of the provided embodiments, the second targeting moiety is selected from the group consisting of a single domain antibody or a single chain variable fragment (scFv). In some of any of the provided embodiments, the single domain antibody is a VHH.

In some of any of the provided embodiments, the first variant paramyxovirus envelope attachment protein and the second variant paramyxovirus envelope attachment protein are the same. In some of any of the provided embodiments, the first paramyxovirus envelope attachment protein and the second paramyxovirus envelope attachment protein are different. In some of any of the provided embodiments, the first paramyxovirus envelope attachment protein is an envelope attachment protein from a Nipah virus, Hendra virus, or Measles virus, or is a variant or biologically active portion thereof of any of the foregoing. In some of any of the provided embodiments, the first paramyxovirus envelope attachment protein is a wild-type paramyxovirus G protein, H protein or HN protein or is a variant or biologically active portion of any of the foregoing. In some of any of the provided embodiments, the first paramyxovirus envelope attachment protein is a wild-type Nipah virus G (NiV-G) protein or is a variant or biologically active portion of a NiV-G.

In some of any of the provided embodiments, the first paramyxovirus envelope attachment protein is a variant NiV-G that is a variant or a biologically active portion of a wild-type NiV-G. In some of any of the provided embodiments, the second paramyxovirus envelope attachment protein is an envelope attachment protein from a Nipah virus, Hendra virus, or Measles virus, or is a variant or biologically active portion of any of the foregoing. In some of any of the provided embodiments, the second paramyxovirus envelope attachment protein is a wild-type paramyxovirus G protein, H protein or HN protein or is a variant or biologically active portion of any of the foregoing. In some of any of the provided embodiments, the second paramyxovirus envelope attachment protein is a wild-type Nipah virus G (NiV-G) protein or is a variant or a biologically active portion of a NiV-G. In some of any of the provided embodiments, the second paramyxovirus envelope attachment protein is a variant NiV-G that is a variant or a biologically active portion of a wild-type NiV-G. In some of any of the provided embodiments, the second paramyxovirus envelope attachment protein is a variant paramyxovirus envelope glycoprotein from a Nipah virus, Hendra virus, or Measles virus or a biologically active portion thereof. In some of any of the provided embodiments, the second paramyxovirus envelope attachment protein is a variant of a wild-type paramyxovirus G protein, H protein or HN protein or a biologically active portion thereof.

In some of any of the provided embodiments, the variant is a variant NiV-G that is a variant of a wild-type Nipah virus G (NiV-G) protein or a biologically active portion thereof. In some of any of the provided embodiments, the variant NiV-G is truncated by up to 40 contiguous amino acids at or near the N-terminus of the wild-type NiV-G set forth in SEQ ID NO:1. In some of any of the provided embodiments, the variant NiV-G has a truncation of amino acids 2-34 of the wild-type NiV-G set forth in SEQ ID NO:1. In some of any of the provided embodiments, the variant NiV-G exhibits reduced binding to Ephrin B2 or Ephrin B3. In some of any of the provided embodiments, the variant NiV-G comprises: one or more amino acid substitutions corresponding to amino acid substitutions selected from the group consisting of E501A, W504A, Q530A and E533A with reference to numbering set forth in SEQ ID NO: 1. In some of any of the provided embodiments, the variant NiV-G comprises amino acid substitutions E501A, W504A, Q530A and E533A with reference to numbering set forth in SEQ ID NO: 1. In some of any of the provided embodiments, the variant NiV-G has the amino acid sequence set forth in SEQ ID NO: 228 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:228. In some of any of the provided embodiments, the variant NiV-G has the amino acid sequence set forth in SEQ ID NO:228. In some of any of the provided embodiments, the at least one paramyxovirus fusion (F) protein is an F protein from a henipavirus or is a biologically active portion thereof or variant thereof.

In some of any of the provided embodiments, the henipavirus is a Hendra virus. In some of any of the provided embodiments, the henipavirus is a Nipah virus. In some of any of the provided embodiments, the paramyxovirus F protein is a wild-type NiV-F protein or a variant or a biologically active portion thereof. In some of any of the provided embodiments, the paramyxovirus F protein is a variant NiV-F that is a variant or a biologically active portion of a wild-type NiV-F protein. In some of any of the provided embodiments, the variant NiV-F is truncated by up to22 contiguous amino acids the at the C-terminus of the wild-type NiV-F set forth in SEQ ID NO:235, optionally not include the initial methionine. In some of any of the provided embodiments, the variant NiV-F protein is a truncated NiV-F that lacks amino acids 525-546 of SEQ ID NO:235. In some of any of the provided embodiments, the variant NiV-F has the amino acid sequence set forth in SEQ ID NO: 227 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 227. In some of any of the provided embodiments, the variant NiV-F has the amino acid sequence set forth in SEQ ID NO:227.

In some of any of the provided embodiments, the paramyxovirus F protein is an F0 precursor or is a proteolytically cleaved form thereof comprising F1 and F2 subunits. In some of any of the provided embodiments, the proteolytically cleaved form is a cathepsin L cleavage product. In some of any of the provided embodiments, the paramyxovirus envelope attachment protein and the first targeting moiety and second targeting moiety are linked via one or more linkers.

In some of any of the provided embodiments, the one or more linkers are one or more peptide linker. In some of any of the provided embodiments, the retargeted attachment protein comprises in order: Paramyxovirus attachment protein-first linker-first targeting moiety-second linker-second targeting moiety.

In some of any of the provided embodiments, the first linker and second linker is independently a peptide linker. In some of any of the provided embodiments, the first linker and second linker are the same. In some of any of the provided embodiments, the first linker and second linker are different. In some of any of the provided embodiments, the peptide linker is 2 to 65 amino acids in length. In some of any of the provided embodiments, the peptide linker is a flexible linker that comprises GS, GGS, GGGGS, GGGGGS or combinations thereof. In some of any of the provided embodiments, the peptide linker is selected from: (GGS)n, wherein n is 1 to 10; (GGGGS)n, wherein n is 1 to 10; or (GGGGGS)n, wherein n is 1 to 6. In some of any of the provided embodiments, the peptide linker is selected from SEQ ID NOs: 589-592.

In some of any of the provided embodiments, the lipid particle further comprises one or more additional paramyxovirus envelope attachment glycoproteins embedded in the lipid bilayer. In some of any of the provided embodiments, the one or more additional paramyxovirus envelope attachment glycoproteins is a retargeted attachment protein comprising a paramyxovirus envelope attachment protein and a further targeting moiety. In some of any of the provided embodiments, the at least one paramyxovirus fusion (F) protein exhibits fusogenic activity with a target cell upon binding of at least one paramyxovirus envelope attachment protein to the target molecule on the target cell.

In some of any of the provided embodiments, the lipid particle comprises a viral nucleic acid. In some of any of the provided embodiments, the viral nucleic acid comprises one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3).

In some of any of the provided embodiments, the lipid particle is a viral vector. In some of any of the provided embodiments, that is a retroviral vector. In some of any of the provided embodiments, that is a lentiviral vector. In some of any of the provided embodiments, the lipid particle is devoid of viral genomic DNA. In some of any of the provided embodiments, the lipid particle is a viral like particle. In some of any of the provided embodiments, the lipid particle is a retroviral-like particle. In some of any of the provided embodiments, the lipid particle is a viral like particle. In some of any of the provided embodiments, the lipid particle is a lentiviral-like particle.

In some of any of the provided embodiments, the lipid particle is produced as a preparation with increased titer compared to a reference lipid particle preparation that is similarly produced but with only the first retargeted attachment protein. In some of any of the provided embodiments, the lipid particle is produced as a preparation with increased titer compared to a reference lipid particle preparation that is similarly produced but without the second paramyxovirus envelope attachment protein that is a variant paramyxovirus envelope attachment protein comprising one or more substitutions to reduce the native tropism relative to the wild-type paramyxovirus envelope attachment protein not comprising the one or more substitutions. In some of any of the provided embodiments, the second paramyxovirus envelope attachment protein that is a variant paramyxovirus envelope attachment protein comprises one or more amino acid substitutions corresponding to amino acid substitutions selected from the group consisting of E501A, W504A, Q530A and E533A with reference to numbering set forth in SEQ ID NO:1. In some of any of the provided embodiments, the lipid particle is produced in suspension culture as a preparation with increased titer compared to a reference lipid particle preparation that is similarly produced but with only the first retargeted attachment protein.

In some of any of the provided embodiments, the titer is increased by at or greater than 1.2-fold, 1.3-fold, 1.4-fold, 1.5-fold, 1.6-fold, 1.7-fold, 1.8-fold, 2-fold, 2.5-fold, 3-fold, 4-fold, 5-fold, 6-fold, 7-fold, 8-fold, 9-fold, 10-fold, or more.

In some of any of the provided embodiments, the lipid particle further comprises an exogenous agent for delivery to a target cell. In some of any of the provided embodiments, the exogenous agent is present in the lumen. In some of any of the provided embodiments, the exogenous agent is a protein or a nucleic acid, optionally wherein the nucleic acid is a DNA or RNA. In some of any of the provided embodiments, the exogenous agent is a nucleic acid encoding a cargo for delivery to the target cell. In some of any of the provided embodiments, the exogenous agent is or encodes a therapeutic agent, a diagnostic agent or a genome-modifying enzyme. In some of any of the provided embodiments, the exogenous agent encodes a membrane protein, optionally wherein the membrane protein is an antigen receptor for targeting cells expressed by or associated with a disease or condition. In some of any of the provided embodiments, the membrane protein is a chimeric antigen receptor (CAR). In some of any of the provided embodiments, the exogenous agent is a nucleic acid comprising a payload gene for correcting a genetic deficiency, optionally a genetic deficiency in the target cell, optionally wherein the genetic deficiency is associated with a liver cell or a hepatocyte.

In some of any of the provided embodiments, binding of the paramyxovirus envelope attachment protein or biologically active portion thereof to a target molecule expressed on the surface of a target cell mediates fusion of the particle with the target cell and delivery of the exogenous agent to the target cell. In some of any of the provided embodiments, at or greater than 10%, 20%, 30%, 40%, 50%, 60% of the target cells are delivered the exogenous agent. In some of any of the provided embodiments, delivery of the exogenous cell to the target cell is increased compared to a reference particle preparation that is similarly produced but with only a first retargeted attachment protein. In some of any of the provided embodiments, the delivery to the target cell is increased by at or greater than 1.2-fold, 1.3-fold, 1.4-fold, 1.5-fold, 1.6-fold, 1.7-fold, 1.8-fold, 2-fold, 2.5-fold, 3-fold, 4-fold, 5-fold, 6-fold, 7-fold, 8-fold, 9-fold, 10-fold, or more.

Provided herein is a producer cell comprising (a) a nucleic acid encoding a retargeted attachment protein comprising a paramyxovirus envelope attachment protein linked to (i) a first targeting moiety directed to a first target molecule expressed on the surface of a target cell, and (ii) a second targeting moiety directed to a second target molecule expressed on the surface of a target cell; and (b) a nucleic acid encoding at least one paramyxovirus fusion (F) protein.

In some of any of the provided embodiments, the producer cell further comprises a nucleic acid encoding a second paramyxovirus envelope attachment protein that is a variant paramyxovirus envelope attachment protein comprising one or more amino acid substitutions to reduce the native tropism relative to the wild-type paramyxovirus envelope attachment protein not comprising the one or more amino acid substitutions.

In some of any of the provided embodiments, the cell further comprises a viral nucleic acid(s). In some of any of the provided embodiments, the viral nucleic acid(s) are lentiviral nucleic acids. In some of any of the provided embodiments, the cell is a mammalian cell. In some of any of the provided embodiments, the producer cell is selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells. In some of any of the provided embodiments, the producer cell comprises 293T cells. In some of any of the provided embodiments, the viral nucleic acid(s) lacks one or more genes involved in viral replication. In some of any of the provided embodiments, the viral nucleic acid comprises a nucleic acid encoding a viral packaging protein selected from one or more of Gag, Pol, Rev and Tat. In some of any of the provided embodiments, the viral nucleic acid comprises: one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3).

Provided herein is a method of making a lipid particle, comprising: a) providing a producer cell such as any of the provided producer cells; b) culturing the cell under conditions that allow for production of the lipid particle, and c) separating, enriching, or purifying the lipid particle from the cell, thereby making the lipid particle. In some of any of the provided embodiments, the lipid particle is a pseudotyped lentiviral vector.

Provided herein is a lipid particle produced by any of the provided methods. Also provided herein is a composition comprising any of the provided lipid particles and/or a plurality of any of the provided lipid particles. Provided herein is a method transducing a cell comprising contacting a cell with any of the provided lipid particles and/or compositions. Provided herein is a method of delivering an exogenous agent into a target cell comprising contacting a cell with any of the provided lipid particles and/or compositions with a target cell.

In some of any of the provided embodiments, the contacting is in vitro or ex vivo. In some of any of the provided embodiments, the contacting is in vivo in a subject.

Provided herein is a method of delivering an exogenous agent to a cell in a subject, the method comprising administering to the subject any of the provided lipid particles and/or compositions. In some of any of the provided embodiments, the exogenous agent is or encodes a therapeutic agent for treating a disease or condition in the subject.

Provided herein is a method of treatment, the method comprising administering to a subject any of the provided lipid particles and/or compositions. In some of any of the provided embodiments, the exogenous agent is or encodes a membrane protein, optionally a chimeric antigen receptor, for targeting an antigen associated with a disease or condition in the subject. In some of any of the provided embodiments, the exogenous agent is for use in gene therapy to correct a genetic deficiency or replaces a deficient or missing gene in the subject. In some of any of the provided embodiments, the subject is a human subject.

In some of any of the provided embodiments, the methods delivery a particle (e.g. a lentiviral particle), including a particle containing an exogenous agent, to a hematopoietic stem cell (HSC). In some of any such provided embodiments, the method further comprises administering to the subject one or more agents that stimulate mobilization of bone marrow cells from the bone marrow to the peripheral blood. In some of any of the provided embodiments, the subject has previously been administered one or more agents that stimulate mobilization of bone marrow cells from the bone marrow to the peripheral blood. In some of any of the provided embodiments, the one or more agents that stimulate mobilization are selected from the group consisting of stem cell factor (SCF), small molecule VLA-4 inhibitor BI05192, BOP (N-(benzenesulfonyl)-L-prolyl-L-0-(1-pyrrolidinylcarbonyl) tyrosine), heparin, granulocyte colony-stimulating factor (G-CSF), MGTA-145, and plerixafor (AMD3100). In some of any of the provided embodiments, the one or more agents that stimulate mobilization comprise G-CSF. In some of any of the provided embodiments, the one or more agents that stimulate mobilization comprise plerixafor.

### BRIEF DESCRIPTION OF THE DRAWINGS

FIG. 1A depicts viral vector titer for exemplary tandem CD117 VHH binders and single CD117 VHH binders. Viral titer for vector pseudotyped with a re-targeted tandem fusogen targeting CD117 and a NiV-F protein with and without an additional blinded fusogen (+Gm) are shown for both CD117-overexpressing cells (FIG. 1B) and CD34+ cells (FIG. 1C). FIG. 1D shows primary cell transduction for exemplary tandem binders.

FIG. 2 depicts transduction of CD34+ primary cells via two exemplary CD133 tandem binders.

FIGS. 3A and 3B show transduction for the indicated fusogens. FIG. 3A depicts transduction of SupT1 T cells via two exemplary CD8 tandem binders. FIG. 3B depicts transduction of Pan-T cells comparing a single binder to a tandem binder.

FIG. 4A shows an exemplary protocol for assessing transduction efficiency in long-term humanized mice. FIG. 4B shows percent transduction in bone marrow compartments using tandem fusogens.

A model study design for non-human primates is shown in FIG. 5.

### DETAILED DESCRIPTION

Provided herein is a lipid particle that contains a fusogen protein on the outer surface of the lipid particle that contains two or more targeting moieties (also termed “tandem fusogen”). In some embodiments, the fusogen is embedded in the lipid bilayer of the lipid particle. In some embodiments, the fusogen contains a viral envelope attachment protein that is linked to the two or more targeting moieties. In some embodiments, the at least two targeting moieties are directed against a target molecule or target molecules on the surface of a cell to thereby retarget the attachment protein to the target molecule or target molecules. In some embodiments, the viral envelope attachment protein contains a first targeting moiety directed to a first target molecule and a second targeting moiety directed to a second target molecule. In some embodiments the first and second target molecule are the same. In some embodiments, the first and second target molecule are different. In some embodiments, the attachment protein is fused to the two or more targeting moieties in which one or more peptide linkers connect the attachment protein and the two or more targeting moieties. In some embodiments, the fusogen facilitates the fusion of the lipid particle to a plasma cell membrane of one or more target cell.

In some embodiments, provided herein is a lipid particle in which a retargeted attachment protein includes at least two targeting moieties directed to a target molecule or target molecules on the surface of the cells and the lipid particle also contains a second attachment protein that is a fusion protein (F protein). In some embodiments, the fusogen is composed of a viral attachment protein comprising the two or more targeting moieties and a fusion protein (F protein) that together are a fusogen. In some embodiments, the fusogen may be derived from a paramyxovirus. In some embodiments, the viral attachment protein is a paramyxovirus envelope attachment protein, such as a G, H or HN protein, and the F protein is at least one paramyxovirus fusion (F) protein. In some embodiments, the attachment protein is a variant attachment protein containing one or more mutations (e.g. amino acid substitutions) to reduce or ablate the native tropism relative to the wild-type virus envelope attachment protein (e.g., paramyxovirus envelope attachment protein) not comprising the one or more mutations.

In some aspects, the lipid particle provided herein includes (a) a retargeted attachment protein with (i) a first targeting moiety directed to a first target molecule expressed on the surface of a target cell, and (ii) a second targeting moiety directed to a second target molecule expressed on the surface of a target cell. In some embodiments, the lipid particles, such as viral vectors or viral-like particles, contain one or more attachment proteins (e.g., fusogens). In some embodiments, the lipid particle, e.g. viral vector or viral-like particle, contains an exogenous or overexpressed attachment protein (e.g., fusogen). In some embodiments, the attachment protein is disposed in the lipid bilayer. In some embodiments, the attachment protein (e.g., fusogen) facilitates the fusion of the lipid particle to a membrane. In some embodiments, the membrane is a plasma cell membrane of a target cell. In some embodiments, the lipid particle, such as a viral or non-viral vector, comprising the attachment protein (e.g., fusogen) integrates into the membrane into a lipid bilayer of a target cell. In some embodiments, the attachment protein (e.g., fusogen) results in mixing between lipids in the lipid particle and lipids in the target cell. In some embodiments, the attachment protein (e.g., fusogen) results in formation of one or more pores between the interior of the non-cell particle and the cytosol of the target cell. In some embodiments, the attachment protein (e.g., fusogen) may include a non-mammalian protein, e.g., a viral protein. In some embodiments, a viral fusogen is a Class I viral membrane fusion protein, a Class II viral membrane protein, a Class III viral membrane fusion protein, a viral membrane glycoprotein, or other viral fusion proteins, or a homologue thereof, a fragment thereof, a variant thereof, or a protein fusion comprising one or more proteins or fragments thereof.

In some aspects, the lipid particle provided herein includes (a) a retargeted attachment protein comprising a paramyxovirus envelope attachment protein operably fused in tandem with (i) a first targeting moiety directed to a first target molecule expressed on the surface of a target cell, and (ii) a second targeting moiety directed to a second target molecule expressed on the surface of a target cell; and (b) a paramyxovirus fusion protein. In some embodiments, the retargeted attachment protein comprises a variant paramyxovirus envelope attachment protein comprising one or more mutations to reduce native tropism relative to the wild-type paramyxovirus envelope attachment protein not comprising the one or more mutations.

In some of any of the embodiments, the lipid particle further contains a second viral envelope attachment protein embedded in the lipid bilayer of the lipid particle that is not attached to a targeting moiety. In some embodiments, the second viral envelope attachment protein is a paramyxovirus envelope attachment protein, such as a G, H or HN protein. In some embodiments, the first viral envelope attachment protein and the second viral envelope attachment protein are the same. In particular embodiments, the second viral envelope attachment protein is a variant paramyxovirus envelope attachment protein comprising one or more mutations to reduce native tropism relative to the wild-type paramyxovirus envelope attachment protein not comprising the one or more mutations. a second envelope attachment protein. In such embodiments, the second viral envelope attachment protein is not linked or fused to a non-viral heterologous moiety, such as a targeting moiety.

In some aspects, the lipid particle provided herein includes (a) a retargeted attachment protein comprising a first paramyxovirus envelope attachment protein operably fused in tandem with (i) a first targeting moiety directed to a first target molecule expressed on the surface of a target cell, and (ii) a second targeting moiety directed to a second target molecule expressed on the surface of a target cell; (b) a second paramyxovirus envelope attachment protein; and (b) a paramyxovirus fusion protein. In some embodiments, the first and second paramyxovirus envelope attachment proteins are the same. In some embodiments, each of the first and second paramyxovirus envelope attachment protein is a variant paramyxovirus envelope attachment protein comprising one or more mutations to reduce native tropism relative to the wild-type paramyxovirus envelope attachment protein not comprising the one or more mutations. In such embodiments, the second paramyxovirus envelope attachment protein is not linked or fused to a non-viral heterologous moiety, such as a targeting moiety.

In some embodiments, the at least two targeting moieties, e.g., a first targeting moiety and second targeting moiety, bind a target molecule on the same target cell. In some embodiments, the at least two targeting moieties, e.g., a first targeting moiety and a second targeting moiety, are the same. In some embodiments, the at least two targeting moieties, e.g., a first targeting moiety and a second targeting moiety, are different. In some embodiments, the at least two targeting moieties, e.g., a first targeting moiety and a second targeting moiety, bind to different epitopes of the same target molecule.

In some embodiments, the at least two targeting moieties, e.g., a first targeting moiety and a second targeting moiety, bind to target molecules on the surface of different target cells.

Exemplary target cells include cells present in a blood sample from a subject. In some embodiments, the cells include a leukocyte component. In some embodiments, the target cells include polymorphonuclear cells (also known as PMN, PML, PMNL, or granulocytes), In some embodiments, the target cells include lymphocytes, monocytes, macrophages, dendritic cells, natural killer cells, T cells (e.g. CD4 or CD8 T cells including cytotoxic T lymphocytes) or B cells. In some embodiments, the target cells include hematopoietic stem cells (HSCs).

In some embodiments, the retargeted attachment protein comprises a paramyxovirus envelope attachment protein and (i) a first targeting moiety directed to a first target molecule expressed on the surface of a T cell, and (ii) a second targeting moiety directed to a second target molecule expressed on the surface of a T cell. In some embodiments, the target molecule on a T cell is CD3, CD4 or CD8. In some embodiments, the first and second target molecule are the same. In some embodiments, the first and second target molecule is CD3. In some embodiments, the first and second target molecule is CD4. In some embodiments, the first and second target molecule is CD8. In some embodiments, the first targeting moiety and second targeting moiety are the same. In some embodiments, the first targeting moiety and second targeting moiety are different. In some embodiments, the first and second targeting moiety bind to different epitopes of the same target molecule.

In some embodiments, the retargeted attachment protein comprises a paramyxovirus envelope attachment protein and (i) a first targeting moiety directed to a first target molecule expressed on the surface of a T cell, and (ii) a second targeting moiety directed to a second target molecule expressed on the surface of a hematopoietic stem cell (HSC). In some embodiments, the target molecule on an HSC is CD117 or CD133. In some embodiments, the first and second target molecule are the same. In some embodiments, the first and second target molecule is CD117. In some embodiments, the first and second target molecule is CD133. In some embodiments, the first targeting moiety and second targeting moiety are the same. In some embodiments, the first targeting moiety and second targeting moiety are different. In some embodiments, the first and second targeting moiety bind to different epitopes of the same target molecule.

In some embodiments, the targeting moiety, such as each of the first targeting moiety and the second targeting moiety, are independently selected from the group consisting of an antibody or antigen-binding fragment, an engineered binding domain, a nanobody, a DARPin, an Aptamer, an Affimer, an Affibody, a Knottin, an Avimer, a Monobody, an Anticalin, a Fynomer, and a targeting peptide.

In some embodiments, any of the provided lipid particles also contains at least one paramyxovirus fusion (F) protein or a biologically active portion thereof embedded in the lipid bilayer. In some embodiments, the F protein is from a Paramyxovirus, a Henipavirus (e.g., Hendra (HeV), Nipah (NiV) virus, Cedar henipavirus (CedV), Kumasi virus (KV), Mòjiang virus (MojV), or Langya virus), or is a biologically active portion thereof or is a variant or mutant thereof. In particular embodiments, the F protein is from a Nipah (NiV) virus.

In naturally occurring paramyxoviruses, the fusion (F) and attachment (G, H, or HN) glycoproteins mediate cellular entry of paramyxovirus, such as Nipah virus. In some embodiments, the combination of an F protein, such as a NiV-F protein, and variant NiV-G protein as provided herein is able to mediate cellular entry of a provided lipid particle (e.g. lentiviral vector).

The F protein, such as Nipah Virus F protein, also known as NiV-F, is a class I fusion protein that has structural and functional features in common with fusion proteins of many families (e.g., HIV-1 gp41 or influenza virus hemagglutinin [HA]), such as an ectodomain with a hydrophobic fusion peptide and two heptad repeat regions (White J M et al. 2008. Crit Rev Biochem Mol Biol 43:189-219). F proteins are synthesized as inactive precursors F₀ and are activated by proteolytic cleavage into the two disulfide-linked subunits F₁ and F₂ (Moll M. et al. 2004. J. Virol. 78 (18): 9705-9712).

In some embodiments, the lipid particle comprises a retargeted paramyxovirus envelope attachment protein comprising a G protein. G proteins are attachment proteins of henipavirus (e.g. Nipah virus or Hendra virus) that are type II transmembrane glycoproteins containing an N-terminal cytoplasmic tail, a transmembrane domain, an extracellular stalk, and a globular head (Liu, Q. et al. 2015. Journal of Virology, 89 (3): 1838-1850). The Nipah virus attachment protein, NiV-G, recognizes the receptors EphrinB2 and EphrinB3. Binding of the receptor to NiV-G triggers a series of conformational changes that eventually lead to the triggering of NiV-F, which exposes the fusion peptide of NiV-F, allowing another series of conformational changes that lead to virus-cell membrane fusion (Stone J. A. et al. 2016. J Virol. 90 (23): 10762-10773). EphrinB2 was previously identified as the primary NiV receptor (Negrete et al., 2005), as well as ephrinB3 as an alternate receptor (Negrete et al., 2006). In fact, wild-type NiV-G has a high affinity for ephrinB2 and B3, with affinity binding constants (Kd) in the picomolar range (Negrete et al., 2006) (Kd=0.06 nM and 0.58 nM for cell surface expressed ephrinB2 and B3, respectively). In some embodiments, the G protein is from a Paramyxovirus, a Henipavirus (e.g., Hendra (HeV), Nipah (NiV) virus, Cedar henipavirus (CedV), Kumasi virus (KV), Mòjiang virus (MojV), or Langya virus), or is a biologically active portion thereof or is a variant or mutant thereof. In particular embodiments, the G protein is from a Nipah (NiV) virus.

In some embodiments, the lipid particles can be a virus-like particle, a virus, or a viral vector, such as a lentiviral vector.

In some embodiments, the retargeted paramyxovirus envelope attachment protein comprising a G protein may be further linked to at least two targeting moieties as a retargeted attachment protein to facilitate specific targeting of the lipid particle to a target molecule or target molecules for fusion with a desired target cell or desired target cells. Thus, the provided embodiments, the envelope attachment protein comprising G proteins may be re-targeted as a retargeted attachment protein to any desired cell type for specific targeting of a lipid particle (e.g. lentiviral vector) and, in some cases, specific delivery to a target cell of a transgene or heterologous protein contained therein.

Thus, also provided herein are lipid particles containing a lipid bilayer enclosing a lumen or cavity and a paramyxovirus envelope attachment protein that is retargeted with two or more targeting moieties containing an antigen binding domain or a biologically active portion thereof, such as a single domain antibody (sdAb) variable domain, in which the retargeted G glycoprotein (e.g., tandem G protein) is embedded in the lipid bilayer of the lipid particles. In particular embodiments, the binding domain is an antibody with the ability to bind, such as specifically bind, to a desired target molecule. Exemplary binding domains are described in Section II.

The efficiency of transduction of lipid particles can be improved by engineering mutations in one or both of NiV-F and NiV-G. Several such mutations have been previously described (see, e.g., Lee at al., 2011, Trends in Microbiology). This is useful, for example, for maintaining the specificity and picomolar affinity of NiV-G for ephrinB2 and/or B3. Additionally, mutations in NiV-G that completely abrogate ephrinB2 and B3 binding, but that do not impact the association of this NiV-G with NiV-F, have been identified (Aguilar, et al. J Biol Chem. 2009; 284 (3): 1628-1635; Weise et al. J Virol. 2010; 84 (15): 7634-764; Negrete et al., J Virol. 2007; 81 (19): 10804-10814; Negrete et al. PLOS Pathog. 2006; Guillaume et al., J. Virol 2006, 80 (15) 7546-7554 In some cases, methods to improve targeting of lipid particles can be achieved by fusion of a binding molecule with a G protein (e.g. NiV-G, including a NiV-G with mutations to abrogate Ephrin B2 and Ephrin B3 binding). This does allow for altered G protein tropism allowing for targeting of other desired cell types that are not ephrinB2+ through the addition of the binding molecule directed against a different cell surface molecule. Thus, in provided aspects, a paramyxovirus envelope attachment protein is a variant NiV-G protein that may further contain a mutation to reduce or abrogate binding to Ephrin B2 and/B3. In some embodiments, the mutations can include one or more of mutations E501A, W504A, Q530A and E533A, with reference to numbering of wild-type NiV-G set forth in SEQ ID NO:1.

In some embodiments, the lipid particle (e.g. viral vector) is pseudotyped with a retargeted attachment protein which comprises a retargeted paramyxovirus attachment protein as described herein, such as a tandem NiV-G protein.

It has been reported that the henipavirus F proteins from various species exhibit compatibility with G proteins from other species to trigger fusion (Brandel-Tretheway et al. Journal of Virology. 2019. 93 (13): e00577-19). In some aspects of the provided lipid particles (e.g. lentiviral vector), the F protein is heterologous to the G protein, i.e. the F and G protein or biologically active portions are from different henipavirus species. For example, the G protein is from Hendra virus and the F protein is a NiV-F as described. In other aspects, the F and/or G protein can be retargeted F and/or G protein containing regions of F and/or G proteins from different species of Henipavirus. In some embodiments, switching a region of amino acid residues of the F protein from one species of Henipavirus to another can result in fusion to the G protein of the species comprising the amino acid insertion. (Brandel-Tretheway et al. 2019). In some cases, the chimeric F and/or G protein contains an extracellular domain from one henipavirus species and a transmembrane and/or cytoplasmic domain from a different henipavirus species. For example, the F protein contains an extracellular domain of Hendra virus and a transmembrane/cytoplasmic domain of Nipah virus.

The provided lipid particles, such as lentiviral vectors, exhibit advantages over available envelope-pseudotyped particles. For instance, VSV-G is the most common envelope glycoprotein used for pseudotyping but its broad tropism is often not ideal or desirable for specific target cell delivery, such as is desired for gene therapy or exogenous protein delivery. Further, although alternative envelope proteins may exhibit reduced tropism or may be amenable to linkage to a binding domain for redirected targeting to a desired target cell, the titer of a preparation of lentiviral vectors containing such envelope proteins may be too low to allow for efficient transduction. Thus, alternative approaches are needed. It is found herein that certain duplicate proteins when pseudotyped on a lentiviral vector exhibit high titers.

Also provided are lipid particles, such as targeted lipid particles, additionally containing one or more exogenous agents, such as for delivery of a diagnostic or therapeutic agent to cells, including following in vivo administration to a subject. Also provided herein are methods and uses of the lipid particles, such in diagnostic and therapeutic methods. Also provided are polynucleotides, methods for engineering, preparing, and producing the lipid non-cell particles, compositions containing the particles, and kits and devices containing and for using, producing and administering the particles.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art. Unless indicated otherwise, abbreviations and symbols for chemical and biochemical names is per IUPAC-IUB nomenclature. Unless indicated otherwise, all numerical ranges are inclusive of the values defining the range as well as all integer values in-between.

As used herein, the articles “a” and “an” refer to one or to more than one (i.e. to at least one) of the grammatical object of the article. By way of example, “an element” means one element or more than one element.

As used herein, the term “about” will be understood by persons of ordinary skill in the art and will vary to some extent on the context in which it is used. As used herein, “about” when referring to a measurable value such as an amount, a temporal duration, and the like, is meant to encompass variations of +20% or +10%, more preferably +5%, even more preferably +1%, and still more preferably +0.1% from the specified value, as such variations are appropriate to perform the disclosed methods.

As used herein, “lipid particle” refers to any biological or synthetic particle that contains a bilayer of amphipathic lipids enclosing a lumen or cavity. Typically a lipid particle does not contain a nucleus. Such lipid particles include, but are not limited to, viral particles (e.g. lentiviral particles), virus-like particles, viral vectors (e.g., lentiviral vectors) exosomes, enucleated cells, various vesicles, such as a microvesicle, a membrane vesicle, an extracellular membrane vesicle, a plasma membrane vesicle, a giant plasma membrane vesicle, an apoptotic body, a mitoparticle, a pyrenocyte, or a lysosome. In some embodiments, a lipid particle can be a fusosome. In some embodiments, the lipid particle is not a platelet. In some embodiments, the fusosome is derived from a source cell. A lipid particle also may include an exogenous agent or a nucleic acid encoding an exogenous agent, which may be present in the lumen of the lipid particle.

The terms “viral vector particle” and “viral vector” are used interchangeably herein and refer to a vector for transfer of an exogenous agent (e.g. non-viral or exogenous nucleic acid) into a recipient or target cell and that contains one or more viral structural proteins in addition to at least one non-structural viral genomic component or functional fragment thereof (i.e., a polymerase, an integrase, a protease or other non-structural component). The viral vector thus contains the exogenous agent, such as heterologous nucleic acid that includes non-viral coding sequences, to be transferred into a cell. Examples of viral vectors are retroviral vectors, such as lentiviral vectors.

The term “retroviral vector” refers to a viral vector that contains retroviral nucleic acid or is derived from a retrovirus. A retroviral vector particle includes the following components: a vector genome (retrovirus nucleic acid), a nucleocapsid encapsidating the nucleic acid, and a membrane envelope surrounding the nucleocapsid. Typically, a retroviral vector contains sufficient retroviral genetic information to allow packaging of an RNA genome, in the presence of packaging components, into a viral particle capable of infecting a target cell. Infection of the target cell may include reverse transcription and integration into the target cell genome. A retroviral vector may be a recombinant retroviral vector that is replication defective and lacks genes essential for replication, such as a functional gag-pol and/or env gene and/or other genes essential for replication. A retroviral vector also may be a self-inactivating (SIN) vector.

As used herein, a “lentiviral vector” or LV refers to a viral vector that contains lentiviral nucleic acid or is derived from a lentivirus. A lentiviral vector particle includes the following components: a vector genome (lentivirus nucleic acid), a nucleocapsid encapsidating the nucleic acid, and a membrane surrounding the nucleocapsid. Typically, a lentiviral vector contains sufficient lentiviral genetic information to allow packaging of an RNA genome, in the presence of packaging components, into a viral particle capable of infecting a target cell. Infection of the target cell may include reverse transcription and integration into the target cell genome. A lentiviral vector may be a recombinant lentiviral vector that is replication defective and lacks genes essential for replication, such as a functional gag-pol and/or env gene and/or other genes essential for replication. A lentiviral vector also may be a self-inactivating (SIN) vector.

As used herein, a “retroviral nucleic acid,” refers to a nucleic acid containing at least the minimal sequence requirements for packaging into a retroviral vector, alone or in combination with a helper cell, helper virus, or helper plasmid. In the case of “lentiviral nucleic acid” the nucleic acid refers to at least the minimal sequence requirements for packaging into a lentiviral vector, alone or in combination with a helper cell, helper virus, or helper plasmid. In some embodiments, the viral nucleic acid comprises one or more of (e.g., all of) a 5′ LTR (e.g., to promote integration), U3 (e.g., to activate viral genomic RNA transcription), R (e.g., a Tat-binding region), U5, a 3′ LTR (e.g., to promote integration), a packaging site (e.g., psi (Y)), RRE (e.g., to bind to Rev and promote nuclear export). The viral nucleic acid can comprise RNA (e.g., when part of a virion) or DNA (e.g., when being introduced into a source cell or after reverse transcription in a recipient cell). In some embodiments, the viral nucleic acid is packaged using a helper cell, helper virus, or helper plasmid which comprises one or more of (e.g., all of) gag, pol, and env.

As used herein, “fusosome” refers to a lipid particle containing a bilayer of amphipathic lipids enclosing a lumen or cavity and a fusogen that interacts with the amphipathic lipid bilayer. In some embodiments, the fusosome is a membrane enclosed preparation. In some embodiments, the fusosome is derived from a source cell. A fusosome also may include an exogenous agent or a nucleic acid encoding an exogenous agent, which may be present in the lumen of the fusosome.

As used herein, “fusosome composition” refers to a composition comprising one or more fusosomes.

As used herein, “fusogen” refers to an agent or molecule that creates an interaction between two membrane enclosed lumens. In embodiments, the fusogen facilitates fusion of the membranes. In other embodiments, the fusogen creates a connection, e.g., a pore, between two lumens (e.g., a lumen of a retroviral vector and a cytoplasm of a target cell). In some embodiments, the fusogen comprises a complex of two or more proteins, e.g., wherein neither protein has fusogenic activity alone. In some embodiments, the fusogen comprises a targeting domain. Examples of fusogens include paramyxovirus F and G proteins such as those from Nipah Virus (NiV) and biologically active portions or variants thereof including any as described.

As used herein, a “re-targeted fusogen,” such as a re-targeted G protein, refers to a fusogen that comprises a targeting moiety having a sequence that is not part of the naturally-occurring form of the fusogen in which the targeting moiety targets or binds a molecule on a desired cell type. In embodiments, the fusogen comprises a different targeting moiety relative to the targeting moiety in the naturally-occurring form of the fusogen. In embodiments, the naturally-occurring form of the fusogen lacks a targeting domain, and the re-targeted fusogen comprises a targeting moiety that is absent from the naturally-occurring form of the fusogen. In embodiments, the fusogen is modified to comprise a targeting moiety. In some such embodiments, the attachment of the targeting moiety to a fusogen (e.g. G protein) may be directly or indirectly via a linker, such as a peptide linker. In embodiments, the fusogen comprises one or more sequence alterations outside of the targeting moiety relative to the naturally-occurring form of the fusogen, e.g., in a transmembrane domain, fusogenically active domain, or cytoplasmic domain.

As used herein, a “target cell” refers to a cell of a type to which it is desired that a lipid particle, such as a targeted lipid particle, delivers an exogenous agent. In embodiments, a target cell is a cell of a specific tissue type or class, e.g., an immune effector cell, e.g., a T cell. In some embodiments, a target cell is a diseased cell, e.g., a cancer cell. In some embodiments, the fusogen, e.g., re-targeted fusogen leads to preferential delivery of the exogenous agent to a target cell compared to a non-target cell.

As used herein a “non-target cell” refers to a cell of a type to which it is not desired that a lipid particle delivers an exogenous agent. In some embodiments, a non-target cell is a cell of a specific tissue type or class. In some embodiments, a non-target cell is a non-diseased cell, e.g., a non-cancerous cell. In some embodiments, the fusogen, e.g., re-targeted fusogen leads to lower delivery of the exogenous agent to a non-target cell compared to a target cell.

As used herein a “biologically active portion,” such as with reference to a protein such as a G protein or an F protein, refers to a portion of the protein that exhibits or retains an activity or property of the full-length of the protein. For example, a biologically active portion of an F protein retains fusogenic activity in conjunction with the G protein when each are embedded in a lipid bilayer. A biologically active portion of the G protein retains fusogenic activity in conjunction with an F protein when each is embedded in a lipid bilayer. The retained activity can include 10%-150% or more of the activity of a full-length or wild-type F protein or G protein. Examples of biologically active portions of F and G proteins include proteins with truncations of the cytoplasmic domain, such as any of the described variant NiV-F with a truncated cytoplasmic tail.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table 1. Amino acid substitutions may be introduced into an antibody of interest and the products screened for a desired activity, for example, retained/improved binding.

TABLE 1
Exemplary substitutions

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term, “corresponding to” with reference to positions of a protein, such as recitation that nucleotides or amino acid positions “correspond to” nucleotides or amino acid positions in a disclosed sequence, such as set forth in the Sequence listing, refers to nucleotides or amino acid positions identified upon alignment with the disclosed sequence based on structural sequence alignment or using a standard alignment algorithm, such as the GAP algorithm. For example, corresponding residues of a similar sequence (e.g. fragment or species variant) can be determined by alignment to a reference sequence by structural alignment methods. By aligning the sequences, one skilled in the art can identify corresponding residues, for example, using conserved and identical amino acid residues as guides.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The term “effective amount” as used herein means an amount of a pharmaceutical composition which is sufficient to significantly and positively modify the symptoms and/or conditions to be treated (e.g., provide a positive clinical response). The effective amount of an active ingredient for use in a pharmaceutical composition will vary with the particular condition being treated, the severity of the condition, the duration of treatment, the nature of concurrent therapy, the particular active ingredient(s) being employed, the particular pharmaceutically-acceptable excipient(s) and/or carrier(s) utilized, and like factors with the knowledge and expertise of the attending physician.

An “exogenous agent” as used herein with reference to a lipid particle, such as a viral vector, refers to an agent that is neither comprised by nor encoded in the corresponding wild-type virus or fusosome made from a corresponding wild-type source cell. In some embodiments, the exogenous agent does not naturally exist, such as a protein or nucleic acid that has a sequence that is altered (e.g., by insertion, deletion, or substitution) relative to a naturally occurring protein. In some embodiments, the exogenous agent does not naturally exist in the source cell. In some embodiments, the exogenous agent exists naturally in the source cell but is exogenous to the virus. In some embodiments, the exogenous agent does not naturally exist in the recipient cell. In some embodiments, the exogenous agent exists naturally in the recipient cell, but is not present at a desired level or at a desired time. In some embodiments, the exogenous agent comprises RNA or protein.

As used herein, a “promoter” refers to a cis-regulatory DNA sequence that, when operably linked to a gene coding sequence, drives transcription of the gene. The promoter may comprise a transcription factor binding sites. In some embodiments, a promoter works in concert with one or more enhancers which are distal to the gene.

As used herein, a composition refers to any mixture of two or more products, substances, or compounds, including cells. It may be a solution, a suspension, liquid, powder, a paste, aqueous, non-aqueous or any combination thereof.

As used herein, the term “pharmaceutically acceptable” refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

A “disease” or “disorder” as used herein refers to a condition where treatment is needed and/or desired.

As used herein, the terms “treat,” “treating,” or “treatment” refer to ameliorating a disease or disorder, e.g., slowing or arresting or reducing the development of the disease or disorder or reducing at least one of the clinical symptoms thereof. For purposes of this disclosure, ameliorating a disease or disorder can include obtaining a beneficial or desired clinical result that includes, but is not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total).

The terms “individual” and “subject” are used interchangeably herein to refer to an animal; for example a mammal. The term patient includes human and veterinary subjects. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. The subject can be male or female and can be any suitable age, including infant, juvenile, adolescent, adult, and geriatric subjects. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder. In particular embodiments, the subject is a human, such as a human patient.

As used herein, the terms “nuclear export sequence” (NES) or “nuclear export signal” (NES) refer to a nuclear export signal or other sequence or domain that is present in a protein and capable of targeting the protein for export from the cell nucleus to the cytoplasm through the nuclear pore complex using nuclear transport. A nuclear export domain can be fused (e.g., fused in-frame) with a polypeptide.

As used herein, the terms “nuclear localization sequence” (NLS) or “nuclear localization sequence” (NLS) refer to a nuclear localization signal or other sequence or domain that is present in a protein and capable of targeting the protein for import from the cytoplasm to the cell nucleus through the nuclear pore complex using nuclear transport. A nuclear localization can be fused (e.g., fused in-frame) with a polypeptide.

### I. Lipid Particles Comprising Tandem Fusogens

In some embodiments, the lipid particles, such as viral vectors or viral-like particles, contain one or more attachment proteins (e.g., fusogens). In some embodiments, the lipid particle, e.g. viral vector or viral-like particle, contains an exogenous or overexpressed attachment protein (e.g., fusogen). In some embodiments, the fusogen is disposed in the lipid bilayer. In some embodiments, the fusogen facilitates the fusion of the lipid particle to a membrane.

In some embodiments, the lipid particle comprises an attachment protein that is retargeted, such as an attachment protein that is linked to at least two targeting moieties, such as a first and second targeting moiety.

In some embodiments, the attachment protein (e.g., fusogen) may include a non-mammalian protein, e.g., a viral protein. In some embodiments, a viral fusogen is a Class I viral membrane fusion protein, a Class II viral membrane protein, a Class III viral membrane fusion protein, a viral membrane glycoprotein, or other viral fusion proteins, or a homologue thereof, a fragment thereof, a variant thereof, or a protein fusion comprising one or more proteins or fragments thereof.

In some embodiments, Class I viral membrane fusion proteins include, but are not limited to, Baculovirus F protein, e.g., F proteins of the nucleopolyhedrovirus (NPV) genera, e.g., Spodoptera exigua MNPV (SeMNPV) F protein and Lymantria dispar MNPV (LdMNPV), and paramyxovirus F proteins.

In some embodiments, Class II viral membrane proteins include, but are not limited to, tick bone encephalitis E (TBEV E), Semliki Forest Virus E1/E2.

In some embodiments, Class III viral membrane fusion proteins include, but are not limited to, rhabdovirus G (e.g., fusogenic protein G of the Vesicular Stomatitis Virus (VSV-G)), herpesvirus glycoprotein B (e.g., Herpes Simplex virus 1 (HSV-1) gB)), Epstein Barr Virus glycoprotein B (EBV gB), thogotovirus G, baculovirus gp64 (e.g., Autographa California multiple NPV (AcMNPV) gp64), Baboon endogenous retrovirus envelope glycoprotein (BaEV), and Borna disease virus (BDV) glycoprotein (BDV G).

Examples of other viral attachment proteins e.g., fusogens that are membrane glycoproteins and viral fusion proteins, include, but are not limited to: viral syncytia proteins such as influenza hemagglutinin (HA) or mutants, or fusion proteins thereof; human immunodeficiency virus type 1 envelope protein (HIV-1 ENV), gp120 from HIV binding LFA-1 to form lymphocyte syncytium, HIV gp41, HIV gp160, or HIV Trans-Activator of Transcription (TAT); viral glycoprotein VSV-G, viral glycoprotein from vesicular stomatitis virus of the Rhabdoviridae family; glycoproteins gB and gH-gL of the varicella-zoster virus (VZV); murine leukemia virus (MLV)-10A1; Gibbon Ape Leukemia Virus glycoprotein (GaLV); type G glycoproteins in Rabies, Mokola, vesicular stomatitis virus and Togaviruses; murine hepatitis virus JHM surface projection protein; porcine respiratory coronavirus spike- and membrane glycoproteins; avian infectious bronchitis spike glycoprotein and its precursor; bovine enteric coronavirus spike protein; the F and H, HN or G genes of Measles virus; canine distemper virus, Newcastle disease virus, human parainfluenza virus 3, simian virus 41, Sendai virus and human respiratory syncytial virus; gH of human herpesvirus 1 and simian varicella virus, with the chaperone protein gL; human, bovine and cercopithicine herpesvirus gB; envelope glycoproteins of Friend murine leukemia virus and Mason Pfizer monkey virus; mumps virus hemagglutinin neuraminidase, and glycoproteins F₁ and F₂; membrane glycoproteins from Venezuelan equine encephalomyelitis; paramyxovirus F protein; SIV gp160 protein; Ebola virus G protein; or Sendai virus fusion protein, or a homologue thereof, a fragment thereof, a variant thereof, and a protein fusion comprising one or more proteins or fragments thereof.

Non-mammalian attachment proteins (e.g., fusogens) include viral fusogens, homologues thereof, fragments thereof, and fusion proteins comprising one or more proteins or fragments thereof. Viral fusogens include class I fusogens, class II fusogens, class III fusogens, and class IV fusogens. In embodiments, class I fusogens such as human immunodeficiency virus (HIV) gp41, have a characteristic post fusion conformation with a signature trimer of α-helical hairpins with a central coiled-coil structure. Class I viral fusion proteins include proteins having a central post fusion six-helix bundle. Class I viral fusion proteins include influenza HA, parainfluenza F, HIV Env, Ebola GP, hemagglutinins from orthomyxoviruses, F proteins from paramyxoviruses (e.g. Measles, (Katoh et al. BMC Biotechnology 2010, 10:37)), ENV proteins from retroviruses, and fusogens of filoviruses and coronaviruses. In embodiments, class II viral fusogens such as dengue E glycoprotein, have a structural signature of β-sheets forming an elongated ectodomain that refolds to result in a trimer of hairpins. In embodiments, the class II viral fusogen lacks the central coiled coil. Class II viral fusogen can be found in alphaviruses (e.g., E1 protein) and flaviviruses (e.g., E glycoproteins). Class II viral fusogens include fusogens from Semliki Forest virus, Sinbis, rubella virus, and dengue virus. In embodiments, class III viral fusogens such as the vesicular stomatitis virus G glycoprotein, combine structural signatures found in classes I and II. In embodiments, a class III viral fusogen comprises a helices (e.g., forming a six-helix bundle to fold back the protein as with class I viral fusogens), and β sheets with an amphiphilic fusion peptide at its end, reminiscent of class II viral fusogens. Class III viral fusogens can be found in rhabdoviruses and herpesviruses. In embodiments, class IV viral fusogens are fusion-associated small transmembrane (FAST) proteins (doi: 10.1038/sj.emboj.7600767, Nesbitt, Rae L., “Targeted Intracellular Therapeutic Delivery Using Liposomes Formulated with Multifunctional FAST proteins” (2012). Electronic Thesis and Dissertation Repository. Paper 388), which are encoded by nonenveloped reoviruses. In embodiments, the class IV viral fusogens are sufficiently small that they do not form hairpins (doi: 10.1146/annurev-cellbio-101512-122422, doi: 10.1016/j.devcel.2007.12.008).

Additional exemplary attachment proteins e.g., fusogens are disclosed in U.S. Pat. No. 9,695,446, US 2004/0028687, U.S. Pat. Nos. 6,416,997, 7,329,807, US 2017/0112773, US 2009/0202622, WO 2006/027202, and US 2004/0009604, the entire contents of all of which are hereby incorporated by reference.

In some embodiments, the attachment protein (e.g., fusogen) is any of the fusogenic moieties described in WO2017/182585; WO2022/164935; WO2021/076788; Hamilton et al. bioRxiv 2022.08.24.505004; Nikolic et al. Nat Commun 9, 1029 (2018); Dobson et al. Nat. Methods. 19, 449-460 (2022); and Yu et al. bioRxiv 2021.12.13.472464, for instance any of the VSV or variant VSV glycoproteins described therein, such as VSV glycoproteins that have reduced binding to native receptors.

In some embodiments, the attachment protein (e.g., fusogen) is a poxviridae fusogen.

In some embodiments, the lipid particle comprises an attachment protein that is a first paramyxovirus attachment protein that is retargeted, such as a paramyxovirus envelope attachment protein that is linked to at least two targeting moieties, such as a first and second targeting moiety. In some embodiments, the lipid particle further comprises at least one paramyxovirus fusion protein.

In some embodiments, the paramyxovirus envelope attachment proteins and/or retargeted attachment proteins provided herein exhibit fusogenic activity to a target cell, such as to deliver an exogenous agent or nucleic acid exogenous agent to the target cell.

In some embodiments, the paramyxovirus attachment protein is or comprises a hemagglutinin-neuraminidase (HN) from a respiratory paramyxovirus. In some embodiments, the respiratory paramyxovirus is a Sendai virus. The HN glycoproteins of Sendai viruses function to attach to sialic acids via the HN protein, and to mediate cell fusion for entry to cells via the F (fusion) protein. In some embodiments, the paramyxovirus attachment protein is or comprises a HN protein from the murine parainfluenza virus type 1 (See e.g., U.S. Pat. No. 10,704,061).

In some embodiments, the paramyxovirus attachment protein is or comprises a Nipah virus protein G, a measles protein H, a tupaia paramyxovirus H protein, a paramyxovirus G protein, a paramyxovirus H protein, a paramyxovirus HN protein, a Morbillivirus H protein, a respirovirus HN protein, a Sendai HN protein, a rubulavirus HN protein, an avulavirus HN protein, or a derivative thereof. In some embodiments, the paramyxovirus attachment protein is or comprises a sequence chosen from Nipah virus G proteins, measles virus H proteins, tupaia paramyxovirus H proteins, paramyxovirus G proteins and H proteins and HN proteins, Hendra virus G proteins, Henipavirus G proteins, Morbillivirus H proteins, respirovirus HN protein, a Sendai virus HN protein, rubulavirus HN proteins, or avulavirus HN proteins, or a derivative thereof, or any combination thereof.

### A. Paramyxovirus Attachment Proteins

In some embodiments, the lipid particles provided herein comprise a paramyxovirus envelope attachment protein, a first paramyxovirus envelope attachment protein, and/or a second paramyxovirus envelope attachment protein. In some embodiments, the paramyxovirus attachment protein is retargeted. In some embodiments, the paramyxovirus envelope attachment protein may be an envelope glycoprotein G, H and/or HN of the Paramyxoviridae family.

In some embodiments, the lipid particles provided herein comprise a first paramyxovirus envelope attachment protein, a second paramyxovirus envelope attachment protein, and a third paramyxovirus envelope attachment protein. In some embodiments, each of the first, second, and third paramyxovirus envelope attachment protein may independently be an envelope glycoprotein G, H and/or HN of the Paramyxoviridae family.

In some embodiments, the lipid particles provided herein comprise a first paramyxovirus envelope attachment protein, a second paramyxovirus envelope attachment protein, a third paramyxovirus envelope attachment protein, and one or more additional paramyxovirus envelope attachment proteins, such as a fourth paramyxovirus envelope attachment protein, or a fourth and fifth paramyxovirus envelope attachment protein, or a fourth, fifth, and sixth paramyxovirus envelope attachment protein, or beyond. In some embodiments, each of the paramyxovirus envelope attachment proteins may independently be an envelope glycoprotein G, H and/or HN of the Paramyxoviridae family.

### 1. G Proteins

In some embodiments, the paramyxovirus envelope attachment protein, first paramyxovirus envelope attachment protein, and/or second paramyxovirus envelope attachment protein and/or the third paramyxovirus envelope attachment protein and/or the fourth paramyxovirus envelope attachment protein and/or the fifth paramyxovirus envelope attachment protein and/or the sixth paramyxovirus envelope attachment protein, and/or any additional paramyxovirus envelope attachment protein is an attachment glycoprotein G (G protein) or biologically active portion thereof. In some embodiment, the retargeted attachment protein comprises a first paramyxovirus envelope attachment protein G.

In some embodiments, the lipid particle comprises a retargeted attachment protein, a first retargeted attachment protein, and/or second retargeted attachment protein exposed on the surface of the targeted lipid particle. In some embodiments, the lipid particle further comprises a third retargeted attachment protein exposed on the surface of the targeted lipid particle. In some embodiments, the lipid particle further comprises a third retargeted attachment protein and a fourth retargeted attachment protein exposed on the surface of the targeted lipid particle. In some embodiments, the lipid particle further comprises a third retargeted attachment protein, a fourth retargeted attachment protein, and a fifth retargeted attachment protein exposed on the surface of the targeted lipid particle. In some embodiments, the lipid particle further comprises a third retargeted attachment protein, a fourth retargeted attachment protein, a fifth retargeted attachment protein, and one or more additional retargeted attachment proteins, exposed on the surface of the targeted lipid particle. In some embodiments, the retargeted attachment protein is or comprises a paramyxovirus attachment protein, wherein the paramyxovirus attachment protein is an attachment glycoprotein G (G protein) or biologically active portion thereof. In some embodiments, the retargeted attachment protein is or comprises a paramyxovirus attachment protein, wherein the paramyxovirus attachment protein is an attachment glycoprotein G (G protein) or biologically active portion thereof, and comprises a targeting moiety directed to a target molecule, e.g., a binding domain or a binding agent, expressed on the surface of a target cell.

The envelope attachment G proteins are type II transmembrane glycoproteins containing an N-terminal cytoplasmic tail (e.g. corresponding to amino acids 1-49 of SEQ ID NO:1), a transmembrane domain (e.g. corresponding to amino acids 50-70 of SEQ ID NO:1), and an extracellular domain containing an extracellular stalk (e.g. corresponding to amino acids 71-187 of SEQ ID NO:1), and a globular head (corresponding to amino acids 188-602 of SEQ ID NO:1). The N-terminal cytoplasmic domain is within the inner lumen of the lipid bilayer and the C-terminal portion is the extracellular domain that is exposed on the outside of the lipid bilayer. Regions of the stalk in the C-terminal region (e.g. corresponding to amino acids 71-187 of SEQ ID NO: 1) have been shown to be involved in interactions with F protein and triggering of F protein fusion (Liu et al. 2015 J of Virology 89:1838). In wild-type G protein, the globular head mediates receptor binding to henipavirus entry receptors Ephrin B2 and Ephrin B3, but is dispensable for membrane fusion (Brandel-Tretheway et al. Journal of Virology. 2019. 93 (13) e00577-19).

In some embodiments herein, tropism of the G protein is altered by linkage of the G protein or biologically active fragment thereof (e.g. cytoplasmic truncation) to a sdAb variable domain. Binding of the G protein to a binding partner can trigger fusion mediated by a compatible paramyxovirus fusion protein (e.g., F protein) or biologically active portion thereof (such as any of the F proteins described in II.B below). G protein sequences disclosed herein are predominantly disclosed as expressed sequences including an N-terminal methionine required for start of translation. As such N-terminal methionines are commonly cleaved co- or post-translationally, the mature protein sequences for all G protein sequences disclosed herein are also contemplated as lacking the N-terminal methionine.

G glycoproteins are highly conserved between henipavirus species. For example, the G protein of NiV and HeV viruses share 79% amino acids identity. Studies have shown a high degree of compatibility among G proteins with F proteins of different species as demonstrated by heterotypic fusion activation (Brandel-Tretheway et al. Journal of Virology. 2019). As described, a lipid particle can contain at least two envelope attachment proteins (e.g., co-fusogens). In particular embodiments, the F protein or the functionally active variant or biologically active portion thereof retains fusogenic activity in conjunction with the at least two envelope attachment proteins (e.g., co-fusogens that are paramyxovirus attachment protein Gs) as provided, such as any set forth below. Fusogenic activity includes the activity of the paramyxovirus fusion protein (e.g., F protein) in conjunction with a G protein to promote or facilitate fusion of two membrane lumens, such as the lumen of the lipid particle provided herein (e.g. having embedded in its lipid bilayer, such as exposed on its surface, at least two G proteins and a F protein), and a cytoplasm of a target cell, e.g. a cell that contains a surface receptor or molecule that is recognized or bound by the G protein.

### Exemplary Henipavirus Protein G Sequences are Provided in Table 2

Table 2. Henipavirus protein G sequence clusters. Column 1, Genbank ID includes the Genbank ID of the whole genome sequence of the virus that is the centroid sequence of the cluster. Column 2, nucleotides of CDS provides the nucleotides corresponding to the CDS of the gene in the whole genome. Column 3, Full Gene Name, provides the full name of the gene including Genbank ID, virus species, strain, and protein name. Column 4, Sequence, provides the amino acid sequence of the gene. Column 5, #Sequences/Cluster, provides the number of sequences that cluster with this centroid sequence. Column 6 provides the SEQ ID numbers for the described sequences.

TABLE 2
Exemplary Henipavirus protein G sequences

| | | Full | | SEQ |
|---|---|---|---|---|
| Genbank | Nucleotides | sequence | | ID |
| ID | of CDS | ID | Sequence | NO |
| AF017149 | 8913-10727 | gb:AF017149\| | MMADSKLVSLNNNLSGKIKDQGKVIKNYYGT | 561 |
| | | Organism:Hendra | MDIKKINDGLLDSKILGAFNTVIALLGSIIIIVM | |
| | | virus\|Strain | NIMIIQNYTRTTDNQALIKESLQSVQQQIKALT | |
| | | Name:UNKNOWN- | DKIGTEIGPKVSLIDTSSTITIPANIGLLGSKISQS | |
| | | AF017149\| | TSSINENVNDKCKFTLPPLKIHECNISCPNPLPF | |
| | | Protein Name: | REYRPISQGVSDLVGLPNQICLQKTTSTILKPR | |
| | | glycoprotein\| | LISYTLPINTREGVCITDPLLAVDNGFFAYSHL | |
| | | Gene Symbol:G | EKIGSCTRGIAKQRIIGVGEVLDRGDKVPSMF | |
| | | | MTNVWTPPNPSTIHHCSSTYHEDFYYTLCAVS | |
| | | | HVGDPILNSTSWTESLSLIRLAVRPKSDSGDYN | |
| | | | QKYIAITKVERGKYDKVMPYGPSGIKQGDTLY | |
| | | | FPAVGFLPRTEFQYNDSNCPIIHCKYSKAENCR | |
| | | | LSMGVNSKSHYILRSGLLKYNLSLGGDIILQFI | |
| | | | EIADNRLTIGSPSKIYNSLGQPVFYQASYSWDT | |
| | | | MIKLGDVDTVDPLRVQWRNNSVISRPGQSQC | |
| | | | PRFNVCPEVCWEGTYNDAFLIDRLNWVSAGV | |
| | | | YLNSNQTAENPVFAVFKDNEILYQVPLAEDDT | |
| | | | NAQKTITDCFLLENVIWCISLVEIYDTGDSVIRP | |
| | | | KLFAVKIPAQCSES | |
| AF212302 | 8943-10751 | gb:AF212302\| | MPAENKKVRFENTTSDKGKIPSKVIKSYYGTM | 1 |
| | | Organism:Nipah | DIKKINEGLLDSKILSAFNTVIALLGSIVIIVMNI | |
| | | virus\|Strain | MIIQNYTRSTDNQAVIKDALQGIQQQIKGLAD | |
| | | Name:UNKNOWN- | KIGTEIGPKVSLIDTSSTITIPANIGLLGSKISQST | |
| | | AF212302\| | ASINENVNEKCKFTLPPLKIHECNISCPNPLPFR | |
| | | Protein Name: | EYRPQTEGVSNLVGLPNNICLQKTSNQILKPKL | |
| | | attachment | ISYTLPVVGQSGTCITDPLLAMDEGYFAYSHL | |
| | | glycoprotein\| | ERIGSCSRGVSKQRIIGVGEVLDRGDEVPSLFM | |
| | | Gene Symbol:G | TNVWTPPNPNTVYHCSAVYNNEFYYVLCAVS | |
| | | | TVGDPILNSTYWSGSLMMTRLAVKPKSNGGG | |
| | | | YNQHQLALRSIEKGRYDKVMPYGPSGIKQGD | |
| | | | TLYFPAVGFLVRTEFKYNDSNCPITKCQYSKP | |
| | | | ENCRLSMGIRPNSHYILRSGLLKYNLSDGENP | |
| | | | KVVFIEISDQRLSIGSPSKIYDSLGQPVFYQASF | |
| | | | SWDTMIKFGDVLTVNPLVVNWRNNTVISRPG | |
| | | | QSQCPRFNTCPEICWEGVYNDAFLIDRINWISA | |
| | | | GVFLDSNQTAENPVFTVFKDNEILYRAQLASE | |
| | | | DTNAQKTITNCFLLKNKIWCISLVEIYDTGDN | |
| | | | VIRPKLFAVKIPEQCT | |
| JQ001776 | 8170-10275 | gb:JQ001776: | MLSQLQKNYLDNSNQQGDKMNNPDKKLSVNFNPL | 562 |
| | | 8170-10275\| | ELDKGQKDLNKSYYVKNKNYNVSNLLNESLHDI | |
| | | Organism:Cedar | KFCIYCIFSLLIIITIINIITISIVITRLKVHE | |
| | | virus\|Strain | ENNGMESPNLQSIQDSLSSLTNMINTEITPRIGI | |
| | | Name:CG1a\| | LVTATSVTLSSSINYVGTKTNQLVNELKDYIT | |
| | | Protein Name: | KSCGFKVPELKLHECNISCADPKISKSAMYST | |
| | | attachment | NAYAELAGPPKIFCKSVSKDPDFRLKQIDYVIP | |
| | | glycoprotein\| | VQQDRSICMNNPLLDISDGFFTYIHYEGINSCK | |
| | | Gene Symbol:G | KSDSFKVLLSHGEIVDRGDYRPSLYLLSSHYH | |
| | | | PYSMQVINCVPVTCNQSSFVFCHISNNTKTLD | |
| | | | NSDYSSDEYYITYFNGIDRPKTKKIPINNMTAD | |
| | | | NRYIHFTFSGGGGVCLGEEFIIPVTTVINTDVFT | |
| | | | HDYCESFNCSVQTGKSLKEICSESLRSPTNSSR | |
| | | | YNLNGIMIISQNNMTDFKIQLNGITYNKLSFGS | |
| | | | PGRLSKTLGQVLYYQSSMSWDTYLKAGFVEK | |
| | | | WKPFTPNWMNNTVISRPNQGNCPRYHKCPEI | |
| | | | CYGGTYNDIAPLDLGKDMYVSVILDSDQLAE | |
| | | | NPEITVFNSTTILYKERVSKDELNTRSTTTSCFL | |
| | | | FLDEPWCISVLETNRFNGKSIRPEIYSYKIPKYC | |
| NC_025256 | 9117-11015 | gb:NC_025256: | MPQKTVEFINMNSPLERGVSTLSDKKTLNQSK | 563 |
| | | 9117-11015\| | ITKQGYFGLGSHSERNWKKQKNQNDHYMTV | |
| | | Organism:Bat | STMILEILVVLGIMFNLIVLTMVYYQNDNINQR | |
| | | Paramyxovirus | MAELTSNITVLNLNLNQLTNKIQREIIPRITLID | |
| | | Eid_hel/GH- | TATTITIPSAITYILATLTTRISELLPSINQKCEFK | |
| | | M74a/GHA/2009\| | TPTLVLNDCRINCTPPLNPSDGVKMSSLATNL | |
| | | Strain | VAHGPSPCRNFSSVPTIYYYRIPGLYNRTALDE | |
| | | Name:BatPV/ | RCILNPRLTISSTKFAYVHSEYDKNCTRGFKYY | |
| | | Eid_hel/GH- | ELMTFGEILEGPEKEPRMFSRSFYSPTNAVNY | |
| | | M74a/GHA/2009\| | HSCTPIVTVNEGYFLCLECTSSDPLYKANLSNS | |
| | | Protein Name: | TFHLVILRHNKDEKIVSMPSFNLSTDQEYVQII | |
| | | glycoprotein\| | PAEGGGTAESGNLYFPCIGRLLHKRVTHPLCK | |
| | | Gene Symbol:G | KSNCSRTDDESCLKSYYNQGSPQHQVVNCLIR | |
| | | | IRNAQRDNPTWDVITVDLTNTYPGSRSRIFGSF | |
| | | | SKPMLYQSSVSWHTLLQVAEITDLDKYQLDW | |
| | | | LDTPYISRPGGSECPFGNYCPTVCWEGTYNDV | |
| | | | YSLTPNNDLFVTVYLKSEQVAENPYFAIFSRD | |
| | | | QILKEFPLDAWISSARTTTISCFMFNNEIWCIAA | |
| | | | LEITRLNDDIIRPIYYSFWLPTDCRTPYPHTGK | |
| | | | MTRVPLRSTYNY | |
| NC_025352 | 8716-11257 | gb:NC_025352: | MATNRDNTITSAEVSQEDKVKKYYGVETAEK | 564 |
| | | 8716-11257\| | VADSISGNKVFILMNTLLILTGAIITITLNITNLT | |
| | | Organism: | AAKSQQNMLKIIQDDVNAKLEMFVNLDQLVK | |
| | | Mojiang | GEIKPKVSLINTAVSVSIPGQISNLQTKFLQKY | |
| | | virus\|Strain | VYLEESITKQCTCNPLSGIFPTSGPTYPPTDKPD | |
| | | Name:Tongguan1\| | DDTTDDDKVDTTIKPIEYPKPDGCNRTGDHFT | |
| | | Protein Name: | MEPGANFYTVPNLGPASSNSDECYTNPSFSIGS | |
| | | attachment | SIYMFSQEIRKTDCTAGEILSIQIVLGRIVDKGQ | |
| | | glycoprotein\| | QGPQASPLLVWAVPNPKIINSCAVAAGDEMG | |
| | | Gene Symbol:G | WVLCSVTLTAASGEPIPHMFDGFWLYKLEPDT | |
| | | | EVVSYRITGYAYLLDKQYDSVFIGKGGGIQKG | |
| | | | NDLYFQMYGLSRNRQSFKALCEHGSCLGTGG | |
| | | | GGYQVLCDRAVMSFGSEESLITNAYLKVNDL | |
| | | | ASGKPVIIGQTFPPSDSYKGSNGRMYTIGDKY | |
| | | | GLYLAPSSWNRYLRFGITPDISVRSTTWLKSQ | |
| | | | DPIMKILSTCTNTDRDMCPEICNTRGYQDIFPL | |
| | | | SEDSEYYTYIGITPNNGGTKNFVAVRDSDGHI | |
| | | | ASIDILQNYYSITSATISCFMYKDEIWCIAITEG | |
| | | | KKQKDNPQRIYAHSYKIRQMCYNMKSATVTV | |
| | | | GNAKNITIRRY | |

In some embodiments, at least one G protein has a sequence set forth in any of SEQ ID NOS: 1, 561, 562, 563, or 564 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to any one of SEQ ID NOS: 1, 561, 562, 563, or 564.

In particular embodiments, the paramyxovirus envelope attachment protein (e.g., G protein) or functionally active variant or biologically active portion is a protein that retains fusogenic activity in conjunction with a paramyxovirus fusion protein (e.g., F protein), such as a NiV-F protein described herein. Fusogenic activity includes the activity of the paramyxovirus envelope attachment protein (e.g., G protein) in conjunction with a paramyxovirus fusion protein (e.g., F protein) to promote or facilitate fusion of two membrane lumens, such as the lumen of the targeted lipid particle having embedded in its lipid bilayer a paramyxovirus fusion protein (e.g., F protein) and paramyxovirus envelope attachment protein (e.g., G protein), and a cytoplasm of a target cell, e.g. a cell that contains a surface receptor or molecule that is recognized or bound by the targeted envelope protein. In some embodiments, the paramyxovirus fusion protein (e.g., F protein) and the paramyxovirus envelope attachment protein (e.g., G protein) are from the same paramyxovirus species (e.g. the same Henipavirus species such as NiV-G and NiV-F).

In some embodiments, at least one G protein or the functionally active variant or biologically active portion thereof binds to Ephrin B2 or Ephrin B3. In some embodiments, the G protein is a variant G protein, such as a truncated G protein as described and retains binding to Ephrin B2 or B3. Reference to retaining binding to Ephrin B2 or B3 includes binding that is similar to the level or degree of binding of the corresponding wild-type G protein, such as set forth in SEQ ID NO: 1, 561, 562, 563, or 564, such as at least 50%, at least 60%, at least 70%, at least 80% or at least 90% of the binding of the wild-type G protein.

In some embodiments, the paramyxovirus envelope attachment protein, the first paramyxovirus envelope attachment protein, and/or the second paramyxovirus envelope attachment protein is a variant G protein that exhibits reduced binding for the native binding partner of a wild-type G protein. In some embodiments, the first paramyxovirus envelope attachment protein, and/or the second paramyxovirus envelope attachment protein and/or the third paramyxovirus envelope attachment protein is a variant G protein that exhibits reduced binding for the native binding partner of a wild-type G protein. In some embodiments, the first paramyxovirus envelope attachment protein, and/or the second paramyxovirus envelope attachment protein and/or the third paramyxovirus envelope attachment protein and/or the fourth paramyxovirus envelope attachment protein is a variant G protein that exhibits reduced binding for the native binding partner of a wild-type G protein. In some embodiments, the first paramyxovirus envelope attachment protein, and/or the second paramyxovirus envelope attachment protein and/or the third paramyxovirus envelope attachment protein and/or the fourth paramyxovirus envelope attachment protein and/or the fifth paramyxovirus envelope attachment protein, and/or one or more additional paramyxovirus envelope attachment proteins is a variant G protein that exhibits reduced binding for the native binding partner of a wild-type G protein. In some embodiments, the variant G protein or the biologically active portion thereof is a variant of wild-type NiV-G and exhibits reduced binding to one or both of the native binding partners Ephrin B2 or Ephrin B3. In some embodiments, the variant G-protein or the biologically active portion, such as a variant NiV-G protein, exhibits reduced binding to the native binding partner. In some embodiments, the reduced binding to Ephrin B2 or Ephrin B3 is reduced by greater than at or about 5%, at or about 10%, at or about 15%, at or about 20%, at or about 25%, at or about 30%, at or about 40%, at or about 50%, at or about 60%, at or about 70%, at or about 80%, at or about 90%, or at or about 100%.

In some embodiments, the mutations (e.g., amino acid substitutions) can improve transduction efficiency. In some embodiments, the mutations allow for specific targeting of other desired cell types that are not Ephrin B2 or Ephrin B3. In some embodiments, the mutations result in at least the partial inability to bind at least one natural receptor, such has reduce the binding to at least one of Ephrin B2 or Ephrin B3. In some embodiments, the mutations described herein interfere with natural receptor recognition.

In some embodiments, at least one G protein contains one or more amino acid substitutions in a residue that is involved in the interaction with one or both of Ephrin B2 and Ephrin B3. In some embodiments, the amino acid substitutions correspond to mutations E501A, W504A, Q530A and E533A with reference to numbering set forth in SEQ ID NO:1. In some embodiments, at least one G protein is a variant G protein containing one or more amino acid substitutions selected from the group consisting of E501A, W504A, Q530A and E533A with reference to numbering set forth in SEQ ID NO:1. In some embodiments, at least one G protein is a variant G protein that contains one or more amino acid substitutions elected from the group consisting of E501A, W504A, Q530A and E533A with reference to SEQ ID NO: 1 and is a biologically active portion thereof containing an N-terminal truncation.

In some embodiments, described herein is a lipid particle comprising a paramyxovirus envelope attachment protein that is not retargeted and comprises one or more amino acid substitutions to reduce the native tropism relative to the wild-type paramyxovirus envelope attachment protein not comprising the one or more amino acid substitutions. In one embodiment, a paramyxovirus envelope attachment protein is a variant paramyxovirus envelope attachment protein comprising one or more amino acid substitutions to reduce the native tropism relative to the wild-type paramyxovirus envelope attachment protein not comprising the one or more amino acid substitutions. In one embodiment, a paramyxovirus envelope attachment protein is a variant paramyxovirus envelope attachment protein comprises one or more amino acid substitutions corresponding to amino acid substitutions selected from the group consisting of E501A, W504A, Q530A and E533A with reference to numbering set forth in SEQ ID NO:1.

In some embodiments, the NiV-G is a variant NiV-G proteins that contain an altered cytoplasmic tail compared to native NiV-G (e.g. SEQ ID NO:5) that are or can be incorporated into a lipid particle, such as a viral particle, including a lentiviral particle or lentiviral-like particle. The cytoplasmic tail of NiV-G corresponds to amino acids 1-45 of SEQ ID NO:5. In some cases, it is understood that the N-terminal methionine of NiV-G, or a variant NiV-G, as described herein can be cleaved and the cytoplasmic tail lacks an initial N-terminal methionine. For instance, in some embodiments, the cytoplasmic tail of wild-type NiV-G may correspond to amino acids 2-45 of SEQ ID NO: 5, and the variant NiV-G protein contains a cytoplasmic tail that is altered compared to amino acids 2-45 of SEQ ID NO:5. In some embodiments, the variant NiV-G contains a modified cytoplasmic tail in which the native cytoplasmic tail is truncated or is replaced by a heterologous cytoplasmic tail.

Non-limiting examples of variant NiV-G proteins, including truncated NiV-G or NiV-G with a altered or modified cytoplasmic tail, are described in WO2013148327, WO2017182585, or PCT/US2022/081872. Further exemplary variant NiV-G proteins are described in Bender et al. 2016 PLOS Pathol 12 (6): e1005641

In some embodiments, at least one G protein is a variant G protein that is a functionally active variant or biologically active portion containing one or more amino acid mutations, such as one or more amino acid insertions, deletions, substitutions or truncations. In some embodiments, the mutations described herein relate to amino acid insertions, deletions, substitutions or truncations of amino acids compared to a reference G protein sequence. In some embodiments, the reference G protein sequence is the wild-type sequence of a G protein or a biologically active portion thereof. In some embodiments, at least one functionally active variant or the biologically active portion thereof is a variant of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein or biologically active portion thereof. In some embodiments, the wild-type G protein has the sequence set forth in any one of SEQ ID NOS: 1, 561, 562, 563, or 564.

In some embodiments, at least one G protein is a variant G protein that is a biologically active portion that is an N-terminally and/or C-terminally truncated fragment of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein. In particular embodiments, the truncation is an N-terminal truncation of all or a portion of the cytoplasmic domain. In some embodiments, at least one variant G protein is a biologically active portion that is truncated and lacks up to 49 contiguous amino acid residues at or near the N-terminus of the wild-type G protein, such as a wild-type G protein set forth in any one of SEQ ID NOS: 1, 561, 562, 563, or 564. In some embodiments, at least one variant G protein is truncated and lacks up to 49 contiguous amino acids, such as up to 49, 48, 47, 46, 45, 44, 43, 42, 41, 40, 30, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 contiguous amino acids at the N-terminus of the wild-type G protein.

In some embodiments, at least one G protein is a wild-type Nipah virus G (NiV-G) protein or a Hendra virus G protein, or is a functionally active variant or biologically active portion thereof. In some embodiments, at least one G protein is a NiV-G protein that has the sequence set forth in SEQ ID NO:1, or is a functional variant or a biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, at least at or about 99% sequence identity to SEQ ID NO:1.

In some embodiments, the variant NiV-G comprises a modified cytoplasmic tail which comprises a truncated cytoplasmic tail from a glycoprotein from the same Nipah virus. In some embodiments, the variant NiV-G contains a modified cytoplasmic tail in which at least a part of the native cytoplasmic tail (e.g. corresponding to amino acids 1-45 of SEQ ID NO:5) is a truncated portion thereof from a glycoprotein from Nipah Virus. In some embodiments, the cytoplasmic tail is a truncated portion thereof that is at least 5 amino acids in length. from or from about 5-44, from or from about 5-40, from or from about 5-30, from or from about 5-20, from or from about 5-10, from or from about 10-44, from or from about 10-40, from or from about 10-30, from or from about 10-20, from or from about 20-44, from or from about 20-40, from or from about 20-30, from or from about 30-44, from or from about 30-40, from or from about 40-44 amino acids in length. In some embodiments, the truncated portion thereof is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43 or 44 amino acids in length. In some embodiments, the variant NiV-G has a cytoplasmic tail that is a truncated NiV-G cytoplasmic tail.

In some embodiments, the truncated NiV-G cytoplasmic tail has a deletion of up to 40, up to 35, up to 30, up to 29, up to 28, up to 27, up to 26, up to 25, up to 24, up to 23, up to 22, up to 21, up to 20, up to 19, up to 18, up to 17, up to 16, up to 15, or up to 14 contiguous amino acid residues at or near the N-terminus of the wild-type NiV-G cytoplasmic tail set forth in SEQ ID NO: 28. In some embodiments, the truncated NiV-G cytoplasmic tail has a deletion of up to 40, up to 35, up to 30, up to 29, up to 28, up to 27, up to 26, up to 25, up to 24, up to 23, up to 22, up to 21, up to 20, up to 19, up to 18, up to 17, up to 16, up to 15, or up to 14 contiguous amino acid residues at or near the N-terminus of the wild-type NiV-G cytoplasmic tail set forth in SEQ ID NO: 4.

In some embodiments, the cytoplasmic tail of NiV-G is set forth in SEQ ID NO:4. In some embodiments, the variant NiV-G has a deletion of between 5 and 41 contiguous amino acid residues at or near the N-terminus of the wild-type NiV-G protein cytoplasmic tail set forth in SEQ ID NO: 4. In some embodiments, the variant NiV-G has a deletion of between 26 and 40 contiguous amino acid residues at or near the N-terminus of the wild-type NiV-G protein cytoplasmic tail set forth in SEQ ID NO: 4.

In some embodiments, at least one G protein is a variant NiV-G protein that is a biologically active portion of a wild-type NiV-G. In some embodiments, the biologically active portion is an N-terminally truncated fragment. In some embodiments, the variant NiV-G protein is truncated and lacks up to 5 contiguous amino acid residues at or near the N-terminus of the wild-type NiV-G protein, such as compared to wild-type NiV-G set forth in SEQ ID NO: 1. In some embodiments, the variant NiV-G protein is truncated and lacks up to 10 contiguous amino acid residues at or near the N-terminus of the wild-type NiV-G protein, such as compared to wild-type NiV-G set forth in SEQ ID NO: 1. In some embodiments, the variant NiV-G protein is truncated and lacks up to 15 contiguous amino acid residues at or near the N-terminus of the wild-type NiV-G protein, such as compared to wild-type NiV-G set forth in SEQ ID NO: 1. n some embodiments, the variant NiV-G protein is truncated and lacks up to 20 contiguous amino acid residues at or near the N-terminus of the wild-type NiV-G protein, such as compared to wild-type NiV-G set forth in SEQ ID NO: 1. In some embodiments, the variant NiV-G protein is truncated and lacks up to 25 contiguous amino acid residues at or near the N-terminus of the wild-type NiV-G protein, such as compared to wild-type NiV-G set forth in SEQ ID NO: 1. In some embodiments, the variant NiV-G protein is truncated and lacks up to 30 contiguous amino acid residues at or near the N-terminus of the wild-type NiV-G protein, such as compared to wild-type NiV-G set forth in SEQ ID NO: 1. In some embodiments, the variant NiV-G protein is truncated and lacks up to 35 contiguous amino acid residues at or near the N-terminus of the wild-type NiV-G protein, such as compared to wild-type NiV-G set forth in SEQ ID NO: 1. In some embodiments, the variant NiV-G protein (also called variant NiV-G) contains an N-terminal methionine.

In some embodiments, the variant NiV-G has a cytoplasmic tail deletion of amino acid residues 2-41, 2-40, 2-39, 2-38, 2-37, 2-36, 2-35, 2-34, 2-33, 2-32, 2-31, 2-30, 2-29, 2-28, 2-27, 2-26, 2-25, 2-22, 2-21, 2-16, 2-11, or 2-5 of SEQ ID NO:4. In some embodiments, the cytoplasmic tail is a truncated portion of the Nipah virus cytoplasmic tail set forth in any one of SEQ ID NOS: 6-28. In some embodiments, the cytoplasmic tail is a truncated portion of the Nipah virus cytoplasmic tail set forth in any one of SEQ ID NOS: 6-28 that lacks the N-terminal methionine. In some embodiments, the variant NiV-G has a sequence in which the cytoplasmic tail, such as set forth in any one of SEQ ID NOS: 6-28, is directly linked to the N-terminus of the sequence set forth in SEQ ID NO: 2. In some embodiments, the variant NiV-G has a sequence in which the cytoplasmic tail set forth in any one of SEQ ID NOS: 6-28 is directly linked to the N-terminus of the sequence set forth in SEQ ID NO: 3. In some embodiments, the cytoplasmic tail is set forth in SEQ ID NO: 7, 13, or 19.

In some embodiments, the variant NiV-G comprises the sequence of amino acids set forth in SEQ ID NO: 211, 220 or 221, or a sequence of amino acids that exhibits at least 85% sequence identity, at least 86% sequence identity, at least 87% sequence identity, at least 88% sequence identity, at least 89% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity to any one of SEQ ID NOs: 211, 220 or 221. In some embodiments, the truncated NiV-G cytoplasmic tail is the sequence of amino acids set forth in SEQ ID NO: 211, 220 or 221.

In some embodiments, the variant NiV-G comprises a modified cytoplasmic tail which comprises a heterologous cytoplasmic tail or a truncated portion thereof from a glycoprotein from another virus. In some embodiments, the other virus is a member of the Kingdom Orthornavirae. In some embodiments, the other virus is a member of the family Paramyxoviridae, Rhabdoviridae, Arenaviridae, or Retroviridae. In some embodiments, the other virus is a member of the family Paramyxoviridae.

In some embodiments, the variant NiV-G contains a modified cytoplasmic tail in which at least a part of the native cytoplasmic tail (e.g. corresponding to amino acids 1-45 of SEQ ID NO:5) is replaced by a heterologous cytoplasmic tail or a truncated portion thereof from a glycoprotein from another virus from another virus or viral-associated protein. In some embodiments, the replaced cytoplasmic tail is a heterologous cytoplasmic tail or a truncated portion thereof that is at least 5 amino acids in length. In some embodiments, the replaced heterologous cytoplasmic tail or a truncated portion thereof is from or from about 5-180 amino acids in length, such as from or from about 5-150, from or from about 5-100, from or from about 5-75, from or from about 5-50, from or from about 5-40, from or from about 5-30, from or from about 5-20, from or from about 5-10, from or from about 10-150, from or from about 10-100, from or from about 10-75, from or from about 10-50, from or from about 10-40, from or from about 10-30, from or from about 10-20, from or from about 20-150, from or from about 20-100, from or from about 20-75, from or from about 20-50, from or from about 20-40, from or from about 20-30, from or from about 30-150, from or from about 30-100, from or from about 30-75, from or from about 30-50, from or from about 30-40, from or from about 40-150, from or from about 40-100, from or from about 40-75, from or from about 40-50, from or from about 50-150, from or from about 50-100, from or from about 50-75, from or from about 75-150, from or from about 75-100 or from or from about 100-150 amino acids in length. In some embodiments, the replaced heterologous cytoplasmic tail or a truncated portion thereof is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids in length. In some embodiments, the heterologous cytoplasmic tail or the truncated portion thereof is directly linked to the N-terminus of the sequence set forth in SEQ ID NO: 2.

In some embodiments, the heterologous cytoplasmic tail is a cytoplasmic tail or a truncated portion thereof from a glycoprotein from another virus, such as a paramyxovirus, a retrovirus, a filovirus, a rhabdovirus or an arenavirus. In some embodiments, the virus is a paramyxovirus other than a Nipah virus. For instance, the virus is a measles virus, Bat paramyxovirus, Cedar Virus, Canine Distemper Virus, Sendai virus, Hendra virus, Human Parainfluenza virus, or Newcastle Disease virus. In some embodiments, the replaced heterologous cytoplasmic tail is the native cytoplasmic tail or a truncated portion of the native cytoplasmic tail of another virus, such as a truncated portion of the cytoplasmic tail set forth in any one of SEQ ID NOS: 40-166. In some embodiments, the variant NiV-G has a sequence in which the heterologous cytoplasmic tail or the truncated portion thereof set forth in any one of SEQ ID NOS: 40-166 is directly linked to the N-terminus of the sequence set forth in SEQ ID NO: 2. In some embodiments, the variant NiV-G contains mutations in the extracellular domain that reduce or abrogate binding to an Ephrin B2 or B3 corresponding to one or more of E501A, W504A, Q530A and E533A, with numbering of residues as set forth SEQ ID NO:1. In some embodiments, the variant NiV-G has a sequence in which the heterologous cytoplasmic tail or the truncated portion thereof set forth in any one of SEQ ID NOS: 40-166 is directly linked to the N-terminus of the sequence set forth in SEQ ID NO: 3. In some embodiments, it is understood that the heterologous cytoplasmic tail or the truncated portion thereof may include any sequence set forth in any one of SEQ ID NOS: 40-166 that lacks the N-terminal methionine.

In some embodiments, the virus is a retrovirus. For instance, the virus may be a baboon endogenous virus (BaEV), Gibbon Ape Leukemia virus (GaLV), murine leukemia virus, or human immunodeficiency virus 1 (HIV-1). In some embodiments, the replaced heterologous cytoplasmic tail is the native cytoplasmic tail or a truncated portion of the native cytoplasmic tail of another virus, such as set forth in any one of SEQ ID NOS: 167-168, 174-177, 179-182, or 185-199. In some embodiments, the variant NiV-G has a sequence in which the heterologous cytoplasmic tail or the truncated portion thereof set forth in any one of SEQ ID NOS: 167-168, 174-177, 179-182, or 185-199 is directly linked to the N-terminus of the sequence set forth in SEQ ID NO: 2. In some embodiments, the variant NiV-G contains mutations in the extracellular domain that reduce or abrogate binding to an Ephrin B2 or B3 corresponding to one or more of E501A, W504A, Q530A and E533A, with numbering of residues as set forth SEQ ID NO:1. In some embodiments, the variant NiV-G has a sequence in which the heterologous cytoplasmic tail or the truncated portion thereof set forth in any one of SEQ ID NOS: 167-168, 174-177, 179-182, or 185-199 is directly linked to the N-terminus of the sequence set forth in SEQ ID NO: 3. In some embodiments, it is understood that the heterologous cytoplasmic tail or the truncated portion thereof may include any sequence set forth in any one of SEQ ID NOS: 167-168, 174-177, 179-182, or 185-199 that lacks the N-terminal methionine.

In some embodiments, the virus is a filovirus. For instance, the virus may be an Ebola virus (EboV). In some embodiments, the replaced heterologous cytoplasmic tail is the native cytoplasmic tail or a truncated portion of the native cytoplasmic tail of another virus, such as set forth in any one of SEQ ID NOS: 172 or 173. In some embodiments, the variant NiV-G has a sequence in which the heterologous cytoplasmic tail or the truncated portion thereof set forth in any one of SEQ ID NOS: 172 or 173 is directly linked to the N-terminus of the sequence set forth in SEQ ID NO: 2. In some embodiments, the variant NiV-G contains mutations in the extracellular domain that reduce or abrogate binding to an Ephrin B2 or B3 corresponding to one or more of E501A, W504A, Q530A and E533A, with numbering of residues as set forth SEQ ID NO:1. In some embodiments, the variant NiV-G has a sequence in which the heterologous cytoplasmic tail or the truncated portion thereof set forth in any one of SEQ ID NOS: 172 or 173 is directly linked to the N-terminus of the sequence set forth in SEQ ID NO: 3. In some embodiments, it is understood that the heterologous cytoplasmic tail or the truncated portion thereof may include any sequence set forth in any one of SEQ ID NOS: 172 or 173 that lacks the N-terminal methionine.

In some embodiments, the virus is a rhabdovirus. For instance, the virus may be Cocal vesiculovirus (Cocal) or vesicular stomatitis virus (VSV). In some embodiments, the replaced heterologous cytoplasmic tail is the native cytoplasmic tail or a truncated portion of the native cytoplasmic tail of another virus, such as set forth in any one of SEQ ID NOS: 170, 171, 183, or 184. In some embodiments, the variant NiV-G has a sequence in which the heterologous cytoplasmic tail or the truncated portion thereof set forth in any one of SEQ ID NOS: 70, 171, 183, or 184 is directly linked to the N-terminus of the sequence set forth in SEQ ID NO: 2. In some embodiments, the variant NiV-G contains mutations in the extracellular domain that reduce or abrogate binding to an Ephrin B2 or B3 corresponding to one or more of E501A, W504A, Q530A and E533A, with numbering of residues as set forth SEQ ID NO:1. In some embodiments, the variant NiV-G has a sequence in which the heterologous cytoplasmic tail or the truncated portion thereof set forth in any one of SEQ ID NOS: 70, 171, 183, or 184 is directly linked to the N-terminus of the sequence set forth in SEQ ID NO: 3. In some embodiments, it is understood that the heterologous cytoplasmic tail or the truncated portion thereof may include any sequence set forth in any one of SEQ ID NOS: 70, 171, 183 or 184 that lacks the N-terminal methionine.

In some embodiments, the virus is an arenavirus. For instance, the virus may be Lymphocytic choriomeningitis virus (LCMV). In some embodiments, the replaced heterologous cytoplasmic tail is the native cytoplasmic tail or a truncated portion of the native cytoplasmic tail of another virus, such as set forth in SEQ ID NOS: 178. In some embodiments, the variant NiV-G has a sequence in which the heterologous cytoplasmic tail or the truncated portion thereof set forth in SEQ ID NOS: 178 is directly linked to the N-terminus of the sequence set forth in SEQ ID NO: 2. In some embodiments, the variant NiV-G contains mutations in the extracellular domain that reduce or abrogate binding to an Ephrin B2 or B3 corresponding to one or more of E501A, W504A, Q530A and E533A, with numbering of residues as set forth SEQ ID NO:1. In some embodiments, the variant NiV-G has a sequence in which the heterologous cytoplasmic tail or the truncated portion thereof set forth in SEQ ID NOS: 178 is directly linked to the N-terminus of the sequence set forth in SEQ ID NO: 3. In some embodiments, it is understood that the heterologous cytoplasmic tail or the truncated portion thereof may include any sequence set forth in any one of SEQ ID NOS: 178 that lacks the N-terminal methionine.

In some embodiments, at least one variant NiV-G protein is truncated and lacks up to amino acid 34 at or near the N-terminus of the wild-type NiV-G protein, such as compared to wild-type NiV-G set forth in SEQ ID NO: 1. In some embodiments, the variant NiV-G protein (also called variant NiV-G) contains an N-terminal methionine. In some embodiments, the variant NiV-G protein lacks amino acids 2-34 as compared to wild-type NiV-G set forth in SEQ ID NO:1. In some embodiments, the NiV-G is set forth in SEQ ID NO:228.

In particular embodiments, at least one G protein has the sequence of amino acids set forth in SEQ ID NO: 228, or is a functionally active variant thereof or a biologically active portion thereof that retains binding and/or fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 228 and retains fusogenic activity in conjunction with a variant NiV-F protein as described. In some embodiments, at least one G protein is a variant G protein that comprises the amino acid sequence of SEQ ID NO: 228.

In some embodiments, the variant NiV-G contains a heterologous cytoplasmic tail that is a cytoplasmic tail or a truncated portion thereof from a glycoprotein from CD63. In some embodiments, the heterologous cytoplasmic tail replaces at least a part of the native cytoplasmic tail of NiV-G (e.g. corresponding to amino acids 1-45 of SEQ ID NO:5). In some embodiments, the heterologous tail is a contiguous sequence of 5, 6, 7, 8, 9, 10, 11, 12, 13, 14, 15, 16, 17, 18, 19, 20, 21, 22, 23, 24, or 25 N-terminal amino acids of the native cytoplasmic tail of CD63. In some embodiments, the native cytoplasmic tail of CD63 is set forth in SEQ ID NOs: 200, 201, or 202. In some embodiments, the heterologous cytoplasmic tail is a truncated portion of the CD63 cytoplasmic tail set forth in any one of SEQ ID NOS: 200-205. In some embodiments, the variant NiV-G has a sequence in which the heterologous cytoplasmic tail set forth in any one of SEQ ID NOS: 200-205 is directly linked to the N-terminus of the sequence set forth in SEQ ID NO: 2. In some embodiments, the variant NiV-G has a sequence in which the heterologous cytoplasmic tail set forth in any one of SEQ ID NOS: 200-205 is directly linked to the N-terminus of the sequence set forth in SEQ ID NO: 3.

In some embodiments, the variant NiV-G comprises a modified cytoplasmic tail which comprises a mutated cytoplasmic tail from a glycoprotein from the same Nipah virus. In some embodiments, the variant NiV-G contains a modified cytoplasmic tail in which at least a part of the native cytoplasmic tail (e.g. corresponding to amino acids 1-45 of SEQ ID NO:5) is a mutated portion thereof from a glycoprotein from Nipah Virus. In some embodiments, the cytoplasmic tail is a mutated portion of the Nipah virus cytoplasmic tail set forth in any one of SEQ ID NOS: 29-38. In some embodiments, it is understood that the truncated NiV-G cytoplasmic tail may include the sequence set forth in any one of SEQ ID NOS: 29-38 that lacks the N-terminal methionine. In some embodiments, the variant NiV-G has a sequence in which the cytoplasmic tail set forth in any one of SEQ ID NOS: 29-38 is directly linked to the N-terminus of the sequence set forth in SEQ ID NO: 2. In some embodiments, the variant NiV-G has a sequence in which the cytoplasmic tail set forth in any one of SEQ ID NOS: 29-38 is directly linked to the N-terminus of the sequence set forth in SEQ ID NO: 3.

In some embodiments, any of the provided lipid particles (lentiviral vectors) may also contain an F protein, such as a NiV-F protein, such as a full-length NiV-F protein or a biologically active portion thereof or a variant thereof. For instance, also provided herein are viral particles or viral-like particles, such as lentiviral particles or lentiviral-like particles, that are pseudotyped with any of the provided variant NiV-G proteins and a NiV-F protein, such as a full-length NiV-F protein or a biologically active portion or a variant thereof. Exemplary NiV-F proteins are further described in Section II.B.

In particular embodiments, the paramyxovirus envelope attachment protein, first paramyxovirus envelope attachment protein and/or second paramyxovirus envelope attachment protein, such as at least one G protein or functionally active variant or biologically active portion thereof, is a protein that retains fusogenic activity in conjunction with other retargeted attachment proteins, such as more than one G protein expressed as a multimer on the lipid bi-layer. In particular embodiments, the first paramyxovirus envelope attachment protein and/or the second paramyxovirus envelope attachment protein and/or the third paramyxovirus envelope attachment protein, such as at least one G protein or functionally active variant or biologically active portion thereof, is a protein that retains fusogenic activity in conjunction with other retargeted attachment proteins, such as more than one G protein expressed as a multimer on the lipid bi-layer. In particular embodiments, the first paramyxovirus envelope attachment protein and/or the second paramyxovirus envelope attachment protein and/or the third paramyxovirus envelope attachment protein and/or the fourth paramyxovirus envelope attachment protein, such as at least one G protein or functionally active variant or biologically active portion thereof, is a protein that retains fusogenic activity in conjunction with other retargeted attachment proteins, such as more than one G protein expressed as a multimer on the lipid bi-layer. In particular embodiments, the first paramyxovirus envelope attachment protein and/or the second paramyxovirus envelope attachment protein and/or the third paramyxovirus envelope attachment protein and/or the fourth paramyxovirus envelope attachment protein and/or one or more additional paramyxovirus envelope attachment proteins, such as at least one G protein or functionally active variant or biologically active portion thereof, is a protein that retains fusogenic activity in conjunction with other retargeted attachment proteins, such as more than one G protein expressed as a multimer on the lipid bi-layer. Fusogenic activity includes the activity of the paramyxovirus envelope attachment protein in conjunction with a protein that is a paramyxovirus fusion protein (e.g., an F protein) to promote or facilitate fusion of two membrane lumens, such as the lumen of the targeted lipid particle having embedded in its lipid bilayer at least two paramyxovirus envelope attachment protein and paramyxovirus fusion protein (e.g., F and G proteins), and a cytoplasm of a target cell, e.g. a cell that contains a surface receptor or molecule that is recognized or bound by the targeted envelope protein.

Reference to retaining fusogenic activity includes activity of a lipid particle (e.g. lentiviral vector) containing at least two paramyxovirus envelope attachment protein and paramyxovirus fusion protein (e.g., F and G proteins) that is between at or about 10% and at or about 150% or more of the level or degree of binding of a reference lipid particle (e.g. lentiviral vector) that is similar, such as contains the same variant NiV-F, but that contains the corresponding wild-type G protein, such as set forth in SEQ ID NO: 1. For instance, a lipid particle (e.g. lentiviral vector) that retains fusogenic activity has at least or at least about 10% of the level or degree of fusogenic activity of the reference lipid particle that is similar (such as contains the same variant NiV-F) but that contains the corresponding wild-type G protein, such as at least or at least about 15% of the level or degree of fusogenic activity, at least or at least about 20% of the level or degree of fusogenic activity, at least or at least about 25% of the level or degree of fusogenic activity, at least or at least about 30% of the level or degree of fusogenic activity, at least or at least about 35% of the level or degree of fusogenic activity, at least or at least about 40% of the level or degree of fusogenic activity, at least or at least about 45% of the level or degree of fusogenic activity, at least or at least about 50% of the level or degree of fusogenic activity, at least or at least about 55% of the level or degree of fusogenic activity, at least or at least about 60% of the level or degree of fusogenic activity, at least or at least about 65% of the level or degree of fusogenic activity, at least or at least about 70% of the level or degree of fusogenic activity, at least or at least about 75% of the level or degree of fusogenic activity, at least or at least about 80% of the level or degree of fusogenic activity, at least or at least about 85% of the level or degree of fusogenic activity, at least or at least about 90% of the level or degree of fusogenic activity, at least or at least about 95% of the level or degree of fusogenic activity, at least or at least about 100% of the level or degree of fusogenic activity, or at least or at least about 120% of the level or degree of fusogenic activity.

Reference to retaining fusogenic activity includes activity of a lipid particle (e.g. lentiviral vector) containing at least two paramyxovirus envelope attachment protein and paramyxovirus fusion protein (e.g., F and G proteins) that is between at or about 10% and at or about 150% or more of the level or degree of binding of a reference lipid particle (e.g. lentiviral vector) that is similar, such as contains the same variant NiV-F, but that contains only one of the provided paramyxovirus envelope attachment proteins (e.g., G proteins). For instance, a lipid particle (e.g. lentiviral vector) that retains fusogenic activity has at least or at least about 10% of the level or degree of fusogenic activity of the reference lipid particle that is similar (such as contains the same variant NiV-F) but that contains only one of the provided paramyxovirus envelope attachment proteins, such as at least or at least about 15% of the level or degree of fusogenic activity, at least or at least about 20% of the level or degree of fusogenic activity, at least or at least about 25% of the level or degree of fusogenic activity, at least or at least about 30% of the level or degree of fusogenic activity, at least or at least about 35% of the level or degree of fusogenic activity, at least or at least about 40% of the level or degree of fusogenic activity, at least or at least about 45% of the level or degree of fusogenic activity, at least or at least about 50% of the level or degree of fusogenic activity, at least or at least about 55% of the level or degree of fusogenic activity, at least or at least about 60% of the level or degree of fusogenic activity, at least or at least about 65% of the level or degree of fusogenic activity, at least or at least about 70% of the level or degree of fusogenic activity, at least or at least about 75% of the level or degree of fusogenic activity, at least or at least about 80% of the level or degree of fusogenic activity, at least or at least about 85% of the level or degree of fusogenic activity, at least or at least about 90% of the level or degree of fusogenic activity, at least or at least about 95% of the level or degree of fusogenic activity, at least or at least about 100% of the level or degree of fusogenic activity, or at least or at least about 120% of the level or degree of fusogenic activity.

### a. Mutated Paramyxovirus G Proteins

In some embodiment, the G protein is a Paramyxovirus G glycoprotein (e.g., variant Paramyxovirus G glycoproteins) comprising one or more amino acid mutations that result in decreased glycosylation of the protein. The one or more amino acid mutations, also called deglycosylation mutations, can be one or more amino acid substitutions (also referred to as mutations).

In some embodiments, the mutant Paramyxovirus G glycoprotein comprises an amino acid substitution at one or more amino acid positions that reduce glycosylation of the G glycoprotein. In some embodiments, the one or more amino acid substitutions disrupts an N-linked glycosylation site. In some embodiments, the one or more amino acid substitutions disrupts an O-linked glycosylation site.

In some embodiments, the mutant Paramyxovirus G glycoprotein is derived from Morbillivirus (e.g., measles virus (MeV), canine distemper virus, Cetacean morbillivirus, Peste-des-petits-ruminants virus, Phocine distemper virus, Rinderpest virus), Henipavirus (e.g., Hendra (HeV) virus, Nipah (NiV) virus, a Cedar (CedPV) virus, Mòjiang virus, a Langya virus or bat Paramyxovirus). In some embodiments, the mutant Paramyxovirus G glycoprotein is a mutant of a Paramyxovirus G glycoprotein derived from Nipah virus or Measles virus. In some embodiments, the mutant Paramyxovirus G protein is a mutant of a paramyxovirus G protein selected from the group consisting of SEQ ID NOs: 1, 561-564 or a modified Paramyxovirus G glycoprotein derived from any one of 1, 5, 561-564 containing an altered cytoplasmic tail. In some embodiments, the mutant Paramyxovirus G protein has a sequence of amino acids that has at least 85%, at least 86%, at least 87%, at least 88%, at least 89%, at least 90%, at least 91%, at least 92%, at least 93%, at least 94% or at least 95% to any one of SEQ ID NOs: 1, 5, 561-564 and contains the acid substitution at one or more amino acid positions that reduce glycosylation of the G glycoprotein as provided herein. In some embodiments, the mutant Paramyxovirus G protein that has one or more amino acid mutations that result in decreased glycosylation is a mutant of the truncated NiV-G set forth in SEQ ID NO:228.

The location of precited glycosylation sites can be determined using the sequence of a protein. For example, N-glycosylation often occurs at sites with the sequence N—X-S/T in which “X” is any amino acid except P. Various algorithms and tools are available for prediction of both N- and O-linked glycosylation, including SprintGly (http://sparks-lab.org/server/sprint-gly/), NetNGlyc (https://services.healthtech.dtu.dk/service.php?NetNGlyc-1.0), NetOGlyc (https://services.healthtech.dtu.dk/service.php?NetOGlyc-4.0), and GlycoMine^{struct} (http://glycomine.erc.monash.edu/Lab/GlycoMine_Struct/), and methods described in Pitti et al., Sci. Reports, 9:15975 (2019) and Pakhrin et al., Molecules 26:7314 (2021). Any predicted glycosylation site may be substituted as described herein.

In some embodiments, the Paramyxovirus G glycoprotein to which the deglycosylation mutation is made is a NiV-G set forth in SEQ ID NO: 1 or a modified Nipah G glycoprotein (NiV-G) that has an altered cytoplasmic tail compared to native NiV-G (e.g., SEQ ID NO: 1). In some embodiments, the variant Paramyxovirus G protein has a sequence of amino acids that has at least 85%, at least 86%, at least 87%, at least 88%, at least 89%, at least 90%, at least 91%, at least 92%, at least 93%, at least 94% or at least 95% to SEQ ID NO: 1 and contains the acid substitution at one or more amino acid positions that reduce glycosylation of the G glycoprotein as provided herein. In some embodiments, the Paramyxovirus G glycoprotein to which the deglycosylation mutation is made is a NiV-G set forth in SEQ ID NO: 5 or a modified Nipah G glycoprotein (NiV-G) that has an altered cytoplasmic tail compared to native NiV-G (e.g., SEQ ID NO: 1). In some embodiments, the variant Paramyxovirus G protein has a sequence of amino acids that has at least 85%, at least 86%, at least 87%, at least 88%, at least 89%, at least 90%, at least 91%, at least 92%, at least 93%, at least 94% or at least 95% to SEQ ID NO: 5 and contains the acid substitution at one or more amino acid positions that reduce glycosylation of the G glycoprotein as provided herein.

Exemplary modified NiV-G proteins with altered cytoplasmic tails to which the one or more amino acid substitutions for reducing glycosylation can be incorporated are described in Section II.A.1.

Amino acid positions for substitutions are described herein with positions “corresponding to” positions of a reference sequence. It is understood that the amino acid substitutions are not limited to being made in only the reference sequence but also can be made in similar sequences by identification of residues that align or correspond with the reference positions. For instance, positions “corresponding to” to positions of a protein in a reference sequence can be identified upon alignment of a similar sequence with the referenced sequence based on structural sequence alignment or using a standard alignment algorithm, such as the GAP algorithm. By aligning the sequences, one skilled in the art can identify corresponding residues, for example, using conserved and identical amino acid residues as guides. For instance, amino acid positions for mutations are described herein with reference to the exemplary truncated NiV-G sequence set forth in SEQ ID NO:5; however, similar amino acid positions for mutations as described can be made in other modified NiV-G sequences, such as any as described in Section II.A.1, by sequence alignment and identification of the corresponding residues.

In some embodiments, the one or more amino acid mutations are at positions corresponding to positions 39, 126, 128, 273, 345, 384, 448, and 496 of SEQ ID NO:5. In some embodiments, the variant Paramyxovirus G glycoprotein comprises an amino acid mutation at any one of positions 39, 126, 128, 273, 345, 384, 448, and 496 of SEQ ID NO:5. In some embodiments, the variant Paramyxovirus G glycoprotein comprises two or more amino acid mutations at any of positions corresponding to positions 39, 126, 128, 273, 345, 384, 448, and 496 of SEQ ID NO:5, such as mutations at 2, 3, 4, 5, 7, or 8 of the positions.

In some embodiments, the one or more amino acid mutations is at a position corresponding to position 39 of SEQ ID NO:5. In some embodiments, the one or more amino acid mutations is at a position corresponding to position 126 of SEQ ID NO:5. In some embodiments, the one or more amino acid mutations is at a position corresponding to position 128 of SEQ ID NO:5. In some embodiments, the one or more amino acid mutations is at a position corresponding to position 273 of SEQ ID NO:5. In some embodiments, the one or more amino acid mutations is at a position corresponding to position 345 of SEQ ID NO:5. In some embodiments, the one or more amino acid mutations is at a position corresponding to position 384 of SEQ ID NO:5. In some embodiments, the one or more amino acid mutations is at a position corresponding to position 448 of SEQ ID NO:5. In some embodiments, the one or more amino acid mutations is at a position corresponding to position 496 of SEQ ID NO:5.

In some embodiments, the native amino acid at the position comprising the amino acid mutation is asparagine or serine. In some embodiments, the amino acid mutation is an amino acid substitution. In some embodiments, the mutation is an asparagine to glutamine substitution. In some embodiments, the mutation is a serine to alanine substitution.

In some embodiments, the mutation is an asparagine to glutamine substitution at a position corresponding to position 39 (N39Q) of SEQ ID NO:5. In some embodiments, the mutation is an asparagine to glutamine substitution at a position corresponding to position 126 (N126Q) of SEQ ID NO: 5. In some embodiments, the mutation is an asparagine to glutamine substitution at a position corresponding to position 273 (N273Q) of SEQ ID NO: 5. In some embodiments, the mutation is an asparagine to glutamine substitution at a position corresponding to position 345 (N345Q) of SEQ ID NO: 5. In some embodiments, the mutation is an asparagine to glutamine substitution at a position corresponding to position 384 (N384Q) of SEQ ID NO: 5. In some embodiments, the mutation is an asparagine to glutamine substitution at a position corresponding to position 448 (N448Q) of SEQ ID NO: 5. In some embodiments, the mutation is an asparagine to glutamine substitution at a position corresponding to position 496 (N496Q) of SEQ ID NO: 5.

In some embodiments, the mutation is a serine to alanine substitution at a position corresponding to position 128 (S128A) of SEQ ID NO: 5.

In some embodiments, the G glycoprotein is derived from Nipah virus G protein and the one or more amino acid substitutions are at positions corresponding to positions selected from the group consisting of 39, 126, 128, 273, 345, 384, 448, and 496 of SEQ ID NO: 5. In some embodiments, the one or more amino acid substitutions are selected from N39Q, N126Q, S128A, N273Q, N345Q, N384Q, N448Q, N496Q or any combination thereof. In some embodiments, the G glycoprotein is a mutant NiV-G containing one amino acid substitution from any one of N39Q, N126Q, S128A, N273Q, N345Q, N384Q, N448Q, N496Q. In some embodiments, the G glycoprotein is a mutant NiV-G containing two amino acid substitutions from any two of N39Q, N126Q, S128A, N273Q, N345Q, N384Q, N448Q, N496Q. In some embodiments, the G glycoprotein is a mutant NiV-G containing three amino acid substitutions from any three of N39Q, N126Q, S128A, N273Q, N345Q, N384Q, N448Q, N496Q. In some embodiments, the G glycoprotein is a mutant NiV-G containing four amino acid substitutions from any one of N39Q, N126Q, S128A, N273Q, N345Q, N384Q, N448Q, N496Q. In some embodiments, the G glycoprotein is a mutant NiV-G containing five amino acid substitutions from any one of N39Q, N126Q, S128A, N273Q, N345Q, N384Q, N448Q, N496Q. In some embodiments, the G glycoprotein is a mutant NiV-G containing six amino acid substitutions from any one of N39Q, N126Q, S128A, N273Q, N345Q, N384Q, N448Q, N496Q. In some embodiments, the G glycoprotein is a mutant NiV-G containing seven amino acid substitutions from any one of N39Q, N126Q, S128A, N273Q, N345Q, N384Q, N448Q, N496Q. In some embodiments, the G glycoprotein is a mutant NiV-G containing eight amino acid substitutions from any one of N39Q, N126Q, S128A, N273Q, N345Q, N384Q, N448Q, N496Q. In some embodiments, the one or more amino acid substitutions are in the SEQ ID NO:5 or a or a modified Nipah G glycoprotein (NiV-G) that has an altered cytoplasmic tail compared to native NiV-G (e.g., SEQ ID NO:5). In some embodiments, the amino acid substitutions are in a modified NiV-G protein described in Section II.A. In some embodiments, the amino acid substitutions are in the NiV-G set forth in SEQ ID NO:5.

In some embodiments, the variant Nipah-G protein comprises at least three amino acid substitutions. In some embodiments, the amino acid substitutions are at positions 273, 384, and 496 of SEQ ID NO:5. In some embodiments, the amino acid substitutions are at positions 273, 345, and 496 of SEQ ID NO:5. In some embodiments, the amino acid substitutions are at positions 39, 126, and 128 of SEQ ID NO:5. In some embodiments, the amino acid substitutions are at positions 39, 273, and 345 of SEQ ID NO:5. In some embodiments, the amino acid substitutions are at positions 39, 384, and 448 of SEQ ID NO:5. In some embodiments, the amino acid substitutions are at positions 39, 448, and 496 of SEQ ID NO:5. In some embodiments, the amino acid substitutions are at positions 39, 128, and 273 of SEQ ID NO:5. In some embodiments, the amino acid substitutions are at positions 39, 345, and 384 of SEQ ID NO:5. In some embodiments, the amino acid substitutions are at positions 39, 384, and 448 of SEQ ID NO:5.

In some embodiments, the variant Nipah-G protein comprises at least two amino acid substitutions. In some embodiments, the amino acid substitutions are at positions 273, and 496 of SEQ ID NO: 5. In some embodiments, the amino acid substitutions are at positions 345, and 496 of SEQ ID NO: 5. In some embodiments, the amino acid substitutions are at positions 39 and 128 of SEQ ID NO:5. In some embodiments, the amino acid substitutions are at positions 39, and 345 of SEQ ID NO:5. In some embodiments, the amino acid substitutions are at positions 39, and 448 of SEQ ID NO:5. In some embodiments, the amino acid substitutions are at positions 39 and 496 of SEQ ID NO:5. In some embodiments, the amino acid substitutions are at positions 39 and 273 of SEQ ID NO:5. In some embodiments, the amino acid substitutions are at positions 39 and 384 of SEQ ID NO:5. In some embodiments, the amino acid substitutions are at positions 384 and 448 of SEQ ID NO:5.

In some embodiments, the amino acid substitution is at position 39 of SEQ ID NO:5. In some embodiments, the amino acid substitution is at position 126 of SEQ ID NO:5. In some embodiments, the amino acid substitution is at position 128 of SEQ ID NO:5. In some embodiments, the amino acid substitution is at position 273 of SEQ ID NO:5. In some embodiments, the amino acid substitution is at position 345 of SEQ ID NO:5. In some embodiments, the amino acid substitution is at position 384 of SEQ ID NO:5. In some embodiments, the amino acid substitution is at position 448 of SEQ ID NO:5. In some embodiments, the amino acid substitution is at position 496 of SEQ ID NO:5.

In some embodiments, the mutant Nipah-G protein comprises an asparagine to glutamine substitution at position 39 of SEQ ID NO:5. In some embodiments, the mutant Nipah-G protein comprises an asparagine to glutamine substitution at position 126 of SEQ ID NO:5. In some embodiments, the mutant Nipah-G protein comprises an asparagine to glutamine substitution at position 273 of SEQ ID NO:5. In some embodiments, the mutant Nipah-G protein comprises an asparagine to glutamine substitution at position 345 of SEQ ID NO:5. In some embodiments, the mutant Nipah-G protein comprises an asparagine to glutamine substitution at position 384 of SEQ ID NO:5. In some embodiments, the mutant Nipah-G protein comprises an asparagine to glutamine substitution at position 448 of SEQ ID NO:5. In some embodiments, the mutant Nipah-G protein comprises an asparagine to glutamine substitution at position 496 of SEQ ID NO:5. In some embodiments, the mutant Nipah-G protein comprises a serine to alanine substitution at position 128 of SEQ ID NO:5.

In some embodiments, the mutant Nipah-G protein comprises the sequence selected from the group consisting of any one of SEQ ID NOs: 640-766, such as any exemplary mutant Nipah-G proteins set forth in Table 2A below. In some embodiments, the mutant Nipah-G protein comprises the sequence of SEQ ID NO: 660. In some embodiments, the variant Nipah-G protein comprises the sequence of SEQ ID NO: 663. In some embodiments, the variant Nipah-G protein comprises the sequence of SEQ ID NO: 667.

TABLE 2A
Exemplary Mutated Paramyxovirus Nipah G Proteins

| NIV-G | SEQ | Position | Position | Position | Position | Position | Position | Position |
|---|---|---|---|---|---|---|---|---|
| (Mutated) | ID NO | 39 | 126 | 273 | 345 | 384 | 448 | 496 |
| NivG | 5 | N | N | N | N | N | N | N |
| NivG.690 | 640 | Q | N | N | N | N | N | N |
| NivG.691 | 641 | N | Q | N | N | N | N | N |
| NivG.693 | 642 | N | N | Q | N | N | N | N |
| NivG.694 | 643 | N | N | N | Q | N | N | N |
| NivG.695 | 644 | N | N | N | N | Q | N | N |
| NivG.696 | 645 | N | N | N | N | N | Q | N |
| NivG.697 | 646 | N | N | N | N | N | N | Q |
| NivG.699 | 647 | N | N | N | Q | Q | Q | N |
| NivG.700 | 648 | N | N | N | Q | N | Q | N |
| NivG.701 | 649 | N | N | N | Q | N | N | Q |
| NivG.702 | 650 | Q | Q | Q | Q | Q | Q | Q |
| NivG.740 | 651 | N | N | N | N | N | Q | Q |
| NivG.742 | 652 | N | N | N | N | Q | N | Q |
| NivG.743 | 653 | N | N | N | N | Q | Q | N |
| NivG.744 | 654 | N | N | N | N | Q | Q | Q |
| NivG.748 | 655 | N | N | N | Q | N | Q | Q |
| NivG.749 | 656 | N | N | N | Q | Q | N | N |
| NivG.750 | 657 | N | N | N | Q | Q | N | Q |
| NivG.752 | 658 | N | N | N | Q | Q | Q | Q |
| NivG.753 | 659 | N | N | Q | N | N | N | Q |
| NivG.754 | 660 | N | N | Q | N | N | Q | N |
| NivG.755 | 661 | N | N | Q | N | N | Q | Q |
| NivG.756 | 662 | N | N | Q | N | Q | N | N |
| NivG.757 | 663 | N | N | Q | N | Q | N | Q |
| NivG.758 | 664 | N | N | Q | N | Q | Q | N |
| NivG.759 | 665 | N | N | Q | N | Q | Q | Q |
| NivG.760 | 666 | N | N | Q | Q | N | N | N |
| NivG.761 | 667 | N | N | Q | Q | N | N | Q |
| NivG.762 | 668 | N | N | Q | Q | N | Q | N |
| NivG.763 | 669 | N | N | Q | Q | N | Q | Q |
| NivG.764 | 670 | N | N | Q | Q | Q | N | N |
| NivG.765 | 671 | N | N | Q | Q | Q | N | Q |
| NivG.766 | 672 | N | N | Q | Q | Q | Q | N |
| NivG.767 | 673 | N | N | Q | Q | Q | Q | Q |
| NivG.768 | 674 | N | Q | N | N | N | N | Q |
| NivG.769 | 675 | N | Q | N | N | N | Q | N |
| NivG.770 | 676 | N | Q | N | N | N | Q | Q |
| NivG.771 | 677 | N | Q | N | N | Q | N | N |
| NivG.772 | 678 | N | Q | N | N | Q | N | Q |
| NivG.773 | 679 | N | Q | N | N | Q | Q | N |
| NivG.774 | 680 | N | Q | N | N | Q | Q | Q |
| NivG.775 | 681 | N | Q | N | Q | N | N | N |
| NivG.776 | 682 | N | Q | N | Q | N | N | Q |
| NivG.777 | 683 | N | Q | N | Q | N | Q | N |
| NivG.778 | 684 | N | Q | N | Q | N | Q | Q |
| NivG.779 | 685 | N | Q | N | Q | Q | N | N |
| NivG.780 | 686 | N | Q | N | Q | Q | N | Q |
| NivG.781 | 687 | N | Q | N | Q | Q | Q | N |
| NivG.782 | 688 | N | Q | N | Q | Q | Q | Q |
| NivG.783 | 689 | N | Q | Q | N | N | N | N |
| NivG.784 | 690 | N | Q | Q | N | N | N | Q |
| NivG.785 | 691 | N | Q | Q | N | N | Q | N |
| NivG.786 | 692 | N | Q | Q | N | N | Q | Q |
| NivG.787 | 693 | N | Q | Q | N | Q | N | N |
| NivG.788 | 694 | N | Q | Q | N | Q | N | Q |
| NivG.789 | 695 | N | Q | Q | N | Q | Q | N |
| NivG.790 | 696 | N | Q | Q | N | Q | Q | Q |
| NivG.791 | 697 | N | Q | Q | Q | N | N | N |
| NivG.792 | 698 | N | Q | Q | Q | N | N | Q |
| NivG.793 | 699 | N | Q | Q | Q | N | Q | N |
| NivG.794 | 700 | N | Q | Q | Q | N | Q | Q |
| NivG.795 | 701 | N | Q | Q | Q | Q | N | N |
| NivG.796 | 702 | N | Q | Q | Q | Q | N | Q |
| NivG.797 | 703 | N | Q | Q | Q | Q | Q | N |
| NivG.798 | 704 | N | Q | Q | Q | Q | Q | Q |
| NivG.799 | 705 | Q | N | N | N | N | N | Q |
| NivG.800 | 706 | Q | N | N | N | N | Q | N |
| NivG.801 | 707 | Q | N | N | N | N | Q | Q |
| NivG.802 | 708 | Q | N | N | N | Q | N | N |
| NivG.803 | 709 | Q | N | N | N | Q | N | Q |
| NivG.804 | 710 | Q | N | N | N | Q | Q | N |
| NivG.805 | 711 | Q | N | N | N | Q | Q | Q |
| NivG.806 | 712 | Q | N | N | Q | N | N | N |
| NivG.807 | 713 | Q | N | N | Q | N | N | Q |
| NivG.808 | 714 | Q | N | N | Q | N | Q | N |
| NivG.809 | 715 | Q | N | N | Q | N | Q | Q |
| NivG.810 | 716 | Q | N | N | Q | Q | N | N |
| NivG.811 | 717 | Q | N | N | Q | Q | N | Q |
| NivG.812 | 718 | Q | N | N | Q | Q | Q | N |
| NivG.813 | 719 | Q | N | N | Q | Q | Q | Q |
| NivG.814 | 720 | Q | N | Q | N | N | N | N |
| NivG.815 | 721 | Q | N | 0 | N | N | N | Q |
| NivG.816 | 722 | Q | N | Q | N | N | Q | N |
| NivG.817 | 723 | Q | N | Q | N | N | Q | Q |
| NivG.818 | 724 | Q | N | Q | N | Q | N | N |
| NivG.819 | 725 | Q | N | Q | N | Q | N | Q |
| NivG.820 | 726 | Q | N | Q | N | Q | Q | N |
| NivG.821 | 727 | Q | N | Q | N | Q | Q | Q |
| NivG.822 | 728 | Q | N | Q | Q | N | N | N |
| NivG.823 | 729 | Q | N | Q | Q | N | N | 0 |
| NivG.824 | 730 | Q | N | Q | Q | N | Q | N |
| NivG.825 | 731 | Q | N | Q | Q | N | Q | Q |
| NivG.826 | 732 | Q | N | Q | Q | Q | N | N |
| NivG.827 | 733 | Q | N | Q | Q | Q | N | Q |
| NivG.828 | 734 | Q | N | Q | Q | Q | Q | N |
| NivG.829 | 735 | Q | N | Q | Q | Q | Q | Q |
| NivG.830 | 736 | Q | Q | N | N | N | N | N |
| NivG.831 | 737 | Q | Q | N | N | N | N | Q |
| NivG.832 | 738 | Q | Q | N | N | N | Q | N |
| NivG.833 | 739 | Q | Q | N | N | N | Q | Q |
| NivG.834 | 740 | Q | Q | N | N | Q | N | N |
| NivG.835 | 741 | Q | Q | N | N | Q | N | 0 |
| NivG.836 | 742 | Q | Q | N | N | Q | Q | N |
| NivG.837 | 743 | Q | Q | N | N | Q | Q | Q |
| NivG.838 | 744 | Q | Q | N | Q | N | N | N |
| NivG.839 | 745 | Q | Q | N | Q | N | N | Q |
| NivG.840 | 746 | Q | Q | N | Q | N | Q | N |
| NivG.841 | 747 | Q | Q | N | Q | N | Q | 0 |
| NivG.842 | 748 | Q | Q | N | Q | Q | N | N |
| NivG.843 | 749 | Q | Q | N | Q | Q | N | Q |
| NivG.844 | 750 | Q | Q | N | Q | Q | Q | N |
| NivG.845 | 751 | Q | Q | N | Q | Q | Q | Q |
| NivG.846 | 752 | Q | Q | Q | N | N | N | N |
| NivG.847 | 753 | Q | Q | Q | N | N | N | Q |
| NivG.848 | 754 | Q | Q | Q | N | N | Q | N |
| NivG.849 | 755 | Q | Q | Q | N | N | Q | Q |
| NivG.850 | 756 | Q | Q | Q | N | Q | N | N |
| NivG.851 | 757 | Q | Q | Q | N | Q | N | Q |
| NivG.852 | 758 | Q | Q | Q | N | Q | Q | N |
| NivG.853 | 759 | Q | Q | Q | N | Q | Q | Q |
| NivG.854 | 760 | Q | Q | Q | Q | N | N | N |
| NivG.855 | 761 | Q | Q | Q | Q | N | N | 0 |
| NivG.856 | 762 | Q | Q | Q | Q | N | Q | N |
| NivG.857 | 763 | Q | Q | Q | Q | N | Q | Q |
| NivG.858 | 764 | Q | Q | Q | Q | Q | N | N |
| NivG.859 | 765 | Q | Q | Q | Q | Q | N | Q |
| NivG.860 | 766 | Q | Q | Q | Q | Q | Q | N |

In some embodiments, the Paramyxovirus G glycoprotein to which the deglycosylation mutations is made is a Measles virus H (Mev-H) protein or a modified MeV-H protein that has an altered cytoplasmic tail compared to native MeV-H (e.g., SEQ ID NO:769). In some embodiments, the mutant Paramyxovirus G protein has a sequence of amino acids that has at least 85%, at least 86%, at least 87%, at least 88%, at least 89%, at least 90%, at least 91%, at least 92%, at least 93%, at least 94% or at least 95% to SEQ ID NO: 769 and contains the acid substitution at one or more amino acid positions that reduce glycosylation of the G glycoprotein as provided herein.

In some embodiments, the G glycoprotein is derived from Measles virus H (Mev-H) protein and the one or more amino acid substitutions are at positions corresponding to positions selected from the group consisting of 168, 187, 200, 215, 238 of SEQ ID NO: 769. In some embodiments, the mutant Mev-H protein comprises at least two amino acid substitutions, such as 2, 3, 4, or 5 substitutions at positions 168, 187, 200, 215, 238 of SEQ ID NO: 769.

In some embodiments, the Paramyxovirus G glycoprotein to which the deglycosylation mutations is made is a Canine distemper virus H (CDV-H) protein or a modified CDV-H protein that has an altered cytoplasmic tail compared to native CDV-H (e.g., SEQ ID NO: 770). In some embodiments, the mutant Paramyxovirus G protein has a sequence of amino acids that has at least 85%, at least 86%, at least 87%, at least 88%, at least 89%, at least 90%, at least 91%, at least 92%, at least 93%, at least 94% or at least 95% to SEQ ID NO: 770 and contains the acid substitution at one or more amino acid positions that reduce glycosylation of the G glycoprotein as provided herein.

In some embodiments, the G glycoprotein is derived from Canine distemper virus H (CDV-H) protein and the one or more amino acid substitutions are at positions corresponding to positions selected from the group consisting of 19, 149, 422 of SEQ ID NO: 770. In some embodiments, the variant CDV-H protein comprises at least two amino acid substitutions, such as 2 or 3 substitutions at positions 19, 149, 422 of SEQ ID NO: 770.

### 2. Retargeted Attachment Proteins

In some embodiments, a paramyxovirus envelope attachment protein, such as a G protein (e.g., NiV-G), is further attached or linked in tandem with at least two binding domains that bind to a first and second target molecule respectively to comprise a retargeted attachment protein. For instance, provided in some aspects is a lipid particle that includes a targeted paramyxovirus envelope attachment proteins (e.g., a chimeric attachment G protein) containing any of the provided G proteins described above that is attached (e.g., operably fused in tandem) to a first and second binding domain, in which the retargeted attachment protein (e.g., re-targeted G protein) is exposed on the surface of the targeted lipid particle (e.g. lentiviral vector). In some of any of the provided embodiments, the lipid particle comprises a retargeted attachment protein comprising (i) a paramyxovirus envelope attachment protein; and (ii) a first targeting moiety directed to a first target molecule expressed on the surface of a target cell, and (iii) a second targeting moiety directed to a second target molecule expressed on the surface of a target cell.

In some embodiments, each of the one or more of the paramyxovirus envelope attachment proteins, such as a G protein (e.g., NiV-G), is further attached or linked in tandem to a first and a second targeting moiety, e.g., a first and second binding domain or a binding agent, directed to a first and second target molecule expressed on the surface of a target cell. The binding domains or binding agents can be individually selected from any binding domain or binding agent described herein, e.g., in Section II. Accordingly, in some embodiments, the lipid particle comprises one or more retargeted attachment proteins, wherein each of the one or more retargeted attachment proteins independently comprise: (i) a paramyxovirus envelope attachment protein; and (ii) a first targeting moiety directed to a first target molecule expressed on the surface of a target cell, and (iii) a second targeting moiety directed to a second target molecule expressed on the surface of a target cell. The targeting moieties can be a binding domain or binding agent, such as any binding domain or any binding agent described herein, e.g., in Section II.

In some embodiments, the envelope attachment protein is a retargeted attachment protein containing a henipavirus G protein or a biologically active portion thereof. In some embodiments, the envelope attachment proteins (e.g., G protein) may be retargeted by tandem linkage to a targeting moiety, such as a binding molecule (e.g. antibody or antigen-binding fragment, e.g. sdAb or scFv) that binds to a target cell, such as a retargeted attachment protein. In some embodiments, the retargeted attachment protein and paramyxovirus fusion protein (e.g., G protein and a NiV-F protein provided herein) together exhibit fusogenic activity to a target cell, such as to deliver an exogenous agent or nucleic acid exogenous agent to the target cell.

In some embodiments, the lipid particle comprises at least two retargeted attachment proteins comprising paramyxovirus envelope attachment proteins (e.g., G proteins), wherein at least one is retargeted by tandem linkage (e.g., operable fusion in tandem) to a first and a second targeting moiety, such as a binding molecule (e.g. antibody or antigen-binding fragment, e.g. sdAb or scFv) that binds to a target cell.

In some embodiments, the retargeted attachment protein is retargeted by tandem linkage to a first and second targeting moiety, wherein the targeting moieties are directed to a target molecule expressed on the surface of a target cell. In some embodiments, the retargeted attachment protein is retargeted by tandem linkage to a first and second targeting moiety, wherein the first and second targeting moiety are directed to the same target molecule expressed on the surface of a target cell. In some embodiments, the retargeted attachment protein is retargeted by tandem linkage to a first and second targeting moiety, wherein the first and second targeting moiety are directed to a first and second target molecule expressed on the surface of a target cell that are different. In some embodiments, the targeting one or both of the first target molecule and the second target molecule does not activate or inhibit, induce a phenotype change (for example maturation and/or differentiation), induce proliferation, and/or induce apoptosis of said target cell.

In some embodiments, the lipid particle comprises at least three retargeted attachment proteins comprising paramyxovirus envelope attachment proteins (e.g., G proteins), wherein at least one is retargeted by tandem linkage to a first and second targeting moiety, such as a binding molecule (e.g. antibody or antigen-binding fragment, e.g. sdAb or scFv) that binds to a target cell. In some embodiments, the lipid particle comprises at least three retargeted attachment proteins comprising envelope attachment proteins (e.g., G proteins), wherein at least two are retargeted by tandem linkage to a first and second targeting moiety, such as a binding molecule (e.g. antibody or antigen-binding fragment, e.g. sdAb or scFv) that binds to a target cell. In some embodiments, the lipid particle comprises at least three retargeted attachment proteins comprising envelope attachment proteins (e.g., G proteins), wherein at least three are retargeted by tandem linkage to a first and second targeting moiety, such as a binding molecule (e.g. antibody or antigen-binding fragment, e.g. sdAb or scFv) that binds to a target cell.

In some embodiments, the first, second, and third retargeted attachment proteins are retargeted by tandem linkage to a first and second targeting moiety, wherein the targeting moieties are independently directed to a first and second target molecule expressed on the surface of a target cell. In some embodiments, the first, second, and third retargeted attachment proteins are retargeted by tandem linkage to a first, second, and third targeting moiety, wherein the first and second targeting moiety, or the second and third targeting moiety, or the first and third targeting moiety, or the first, second, and third target moiety, are directed to the same target molecule expressed on the surface of a target cell. In some embodiments, the first, second, and third retargeted attachment proteins are retargeted by tandem linkage to a first, second, and third targeting moiety, wherein the first, second, and third targeting moiety are directed to a first, second, and third target molecule expressed on the surface of a target cell that are different. In some embodiments, the targeting of one, two, or three of the first target molecule, the second target molecule, and the third target molecule does not activate or inhibit, induce a phenotype change (for example maturation and/or differentiation), induce proliferation, and/or induce apoptosis of said target cell.

In some embodiments, the lipid particle comprises at least four or at least five retargeted attachment proteins comprising paramyxovirus envelope attachment proteins (e.g., G proteins), wherein at least one, at least two, at least three, or at least four is retargeted by tandem linkage to a first and second targeting moiety, such as a binding molecule (e.g. antibody or antigen-binding fragment, e.g. sdAb or scFv) that binds to a target cell. In some embodiments, the lipid particle comprises at least four or at least five retargeted attachment proteins comprising envelope attachment proteins (e.g., G proteins), wherein at least two or at least three are retargeted by tandem linkage to a first and second targeting moiety, such as a binding molecule (e.g. antibody or antigen-binding fragment, e.g. sdAb or scFv) that binds to a target cell. In some embodiments, the lipid particle comprises at least four or at least five retargeted attachment proteins comprising envelope attachment proteins (e.g., G proteins), wherein at least four or at least five are retargeted by tandem linkage to a targeting moiety, such as a binding molecule (e.g. antibody or antigen-binding fragment, e.g. sdAb or scFv) that binds to a target cell.

In some embodiments, the paramyxovirus retargeted attachment protein is a targeted envelope protein containing a G protein provided herein. In some embodiments the paramyxovirus retargeted attachment protein comprises at least one envelope attachment proteins (e.g., G protein) that is any of those provided in Section II.A, including NiV-G proteins with cytoplasmic domain modifications, truncated NiV-G cytoplasmic tails, or modified NiV-G cytoplasmic tails.

In some of any embodiments, the retargeted attachment protein comprises (a) a retargeted attachment protein comprising a first paramyxovirus envelope attachment protein operably fused in tandem with (i) a first targeting moiety directed to a first target molecule expressed on the surface of a target cell, and (ii) a second targeting moiety directed to a second target molecule expressed on the surface of a target cell. In some embodiments, the targeting moiety is a binding domain, such as any of the binding domains or binding agents described herein in Section II.A.2, e.g., a T cell binding domain or an HSC binding domain. In some embodiments, the binding domain can be any agent that binds to a cell surface molecule on a target cells. In some embodiments, the binding domain can be an antibody or an antibody portion or fragment. In some embodiments, the binding domain is a single domain antibody (sdAb). In some embodiments, the binding domain is a single chain variable fragment (scFv). The binding domain can be linked directly or indirectly to the G protein. In particular embodiments, the binding domain is linked to the C-terminus (C-terminal amino acid) of the G protein or the biologically active portion thereof. The linkage can be via a peptide linker, such as a flexible peptide linker.

The retargeted attachment protein comprising a first paramyxovirus envelope attachment protein operably fused in tandem with (i) a first targeting moiety directed to a first target molecule expressed on the surface of a target cell, and (ii) a second targeting moiety may be modulated to have different binding strengths. For example, scFvs and antibodies with various binding strengths may be used to alter the fusion activity of the retargeted attachment proteins towards cells that display high or low amounts of the target antigen. For example DARPins with different affinities may be used to alter the fusion activity towards cells that display high or low amounts of the target antigen. Binding domains may also be modulated to target different regions on the target ligand, which will affect the fusion rate with cells displaying the target.

The binding domain may comprise a humanized antibody molecule, intact IgA, IgG, IgE or IgM antibody; bi- or multi-specific antibody (e.g., Zybodies®, etc.); antibody fragments such as Fab fragments, Fab′ fragments, F(ab′) 2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPsTM”); single chain or Tandem diabodies (TandAb®); VHHs; Anticalins®; Nanobodies®; minibodies; BiTE®s; ankyrin repeat proteins or DARPINS®; Avimers®; DARTs; TCR-like antibodies; Adnectins®; Affilins®; Trans-Bodies®; Affibodies®; TrimerX®; MicroProteins; Fynomers®, Centyrins®; and KALBITOR®s. A targeting moiety can also include an antibody or an antigen-binding fragment thereof (e.g., Fab, Fab′, F(ab′) 2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs).

The binding domain may comprise a humanized antibody molecule, intact IgA, IgG, IgE or IgM antibody; bi- or multi-specific antibody (e.g., Zybodies®, etc.); antibody fragments such as Fab fragments, Fab′ fragments, F(ab′) 2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPsTM”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-Bodies®; Affibodies®; TrimerX®; MicroProteins; Fynomers®, Centyrins®; and KALBITOR®s. A targeting moiety can also include an antibody or an antigen-binding fragment thereof (e.g., Fab, Fab′, F(ab′) 2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody or a T cell receptor (TCRs). In some embodiments, the binding domain does not comprise a ligand, a cytokine, or a chemokine,

In some embodiments, the binding domain is a single chain molecule. In some embodiments, the binding domain is a single domain antibody. In some embodiments, the binding domain is a single chain variable fragment. In particular embodiments, the binding domain contains an antibody variable sequence(s) that is human or humanized.

In some embodiments, the binding domain is a single domain antibody. In some embodiments, the single domain antibody can be human or humanized. In some embodiments, the single domain antibody or portion thereof is naturally occurring. In some embodiments, the single domain antibody or portion thereof is synthetic.

In some embodiments, the single domain antibodies are antibodies whose complementary determining regions are part of a single domain polypeptide. In some embodiments, the single domain antibody is a heavy chain only antibody variable domain. In some embodiments, the single domain antibody does not include light chains.

In some embodiments, the heavy chain antibody devoid of light chains is referred to as VHH. In some embodiments, the single domain antibody antibodies have a molecular weight of 12-15 kDa. In some embodiments, the single domain antibody antibodies include camelid antibodies or shark antibodies. In some embodiments, the single domain antibody molecule is derived from antibodies raised in Camelidae species, for example in camel, llama, dromedary, alpaca, vicuna and guanaco. In some embodiments, the single domain antibody is referred to as immunoglobulin new antigen receptors (IgNARs) and is derived from cartilaginous fishes. In some embodiments, the single domain antibody is generated by splitting dimeric variable domains of human or mouse IgG into monomers and camelizing critical residues.

In some embodiments, the single domain antibody can be generated from display libraries, e.g., phage display libraries. In some embodiments, the display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the display library is generated comprising antibody fragments of a non-immunized camelid. In some embodiments, single domain antibodies a library of human single domain antibodies is synthetically generated by introducing diversity into one or more scaffolds.

In some embodiments, the binding domain is a single domain antibody (sdAb). In some embodiments, the binding domain is a single chain variable fragment (scFv). The binding domain can be linked directly or indirectly to the paramyxovirus envelope attachment protein, first paramyxovirus envelope attachment protein, and/or second paramyxovirus envelope attachment protein (e.g., G protein, and/or retargeted attachment protein). In particular embodiments, the binding domain is linked to the C-terminus (C-terminal amino acid) of the G protein or the biologically active portion thereof. The linkage can be via a peptide linker, such as a flexible peptide linker. In particular embodiments, the first and/or second targeting moiety is operably fused in tandem with the paramyxovirus envelope attachment protein.

In some embodiments, the C-terminus of the binding domain is attached to the C-terminus of the G protein or biologically active portion thereof. In some embodiments, the N-terminus of the binding domain is exposed on the exterior surface of the lipid bilayer. In some embodiments, the N-terminus of the binding domain binds to a cell surface molecule of a target cell. In some embodiments, the binding domain specifically binds to a cell surface molecule present on a target cell. In some embodiments, the cell surface molecule is a protein, glycan, lipid or low molecular weight molecule. In some embodiments, the binding domain is one of any binding domains as described above.

In some embodiments, a binding domain (e.g. sdAb or one of any binding domains as described herein) binds to a cell surface antigen of a cell. In some embodiments, a cell surface antigen is characteristic of one type of cell. In some embodiments, a cell surface antigen is characteristic of more than one type of cell.

In some embodiments, the cell surface molecule of a target cell is an antigen or portion thereof. In some embodiments, the single domain antibody or portion thereof is an antibody having a single monomeric domain antigen binding/recognition domain that is able to bind selectively to a specific antigen. In some embodiments, the single domain antibody binds an antigen present on a target cell.

Exemplary cells include polymorphonuclear cells (also known as PMN, PML, PMNL, or granulocytes), stem cells, embryonic stem cells, neural stem cells, mesenchymal stem cells (MSCs), hematopoietic stem cells (HSCs), human myogenic stem cells, muscle-derived stem cells (MuStem), embryonic stem cells (ES or ESCs), limbal epithelial stem cells, cardio-myogenic stem cells, cardiomyocytes, progenitor cells, immune effector cells, lymphocytes, macrophages, dendritic cells, natural killer cells, T cells, cytotoxic T lymphocytes, allogenic cells, resident cardiac cells, induced pluripotent stem cells (iPS), adipose-derived or phenotypic modified stem or progenitor cells, CD133+ cells, aldehyde dehydrogenase-positive cells (ALDH+), umbilical cord blood (UCB) cells, peripheral blood stem cells (PBSCs), neurons, neural progenitor cells, pancreatic beta cells, glial cells, or hepatocytes.

In some embodiments, the target cell is a cell of a target tissue. The target tissue can include liver, lungs, heart, spleen, pancreas, gastrointestinal tract, kidney, testes, ovaries, brain, reproductive organs, central nervous system, peripheral nervous system, skeletal muscle, endothelium, inner ear, or eye.

In some embodiments, the target cell is a muscle cell (e.g., skeletal muscle cell), kidney cell, liver cell (e.g. hepatocyte), or a cardiac cell (e.g. cardiomyocyte). In some embodiments, the target cell is a cardiac cell, e.g., a cardiomyocyte (e.g., a quiescent cardiomyocyte), a hepatoblast (e.g., a bile duct hepatoblast), an epithelial cell, a T cell (e.g. a naive T cell), a macrophage (e.g., a tumor infiltrating macrophage), or a fibroblast (e.g., a cardiac fibroblast).

In some embodiments, the target cell is a tumor-infiltrating lymphocyte, a T cell, a neoplastic or tumor cell, a virus-infected cell, a stem cell, a central nervous system (CNS) cell, a hematopoietic stem cell (HSC), a liver cell or a fully differentiated cell. In some embodiments, the target cell is a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+B cell, a CD19+B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

In some embodiments, the target cell is an antigen presenting cell, an MHC class II+ cell, a professional antigen presenting cell, an atypical antigen presenting cell, a macrophage, a dendritic cell, a myeloid dendritic cell, a plasmacyteoid dendritic cell, a CD11c+ cell, a CD11b+ cell, a splenocyte, a B cell, a hepatocyte, a endothelial cell, or a non-cancerous cell). In some embodiments, the first and second target molecules are present on the same target cell. In some embodiments, the first and second target molecules are present on different cells.

In some embodiments, the binding domain (e.g. sdAb) variable domain binds a cell surface molecule or antigen. In some embodiments, the cell surface molecule is ASGR1, ASGR2, TM4SF5, CD8, CD4, or low density lipoprotein receptor (LDL-R). In some embodiments, the cell surface molecule is ASGR1. In some embodiments, the cell surface molecule is ASGR2. In some embodiments, the cell surface molecule is TM4SF5. In some embodiments, the cell surface molecule is CD8. In some embodiments, the cell surface molecule is CD4. In some embodiments, the cell surface molecule is LDL-R.

In some embodiments, the target cell is a hematopoietic lineage cell. Reference to a “hematopoietic cell” includes blood cells, both from the myeloid and the lymphoid lineage. In particular, the term “hematopoietic cell” includes both undifferentiated or poorly differentiated cells, such as hematopoietic stem cells and progenitor cells, and differentiated cells such as T lymphocytes, B lymphocytes, or dendritic cells. In some embodiments, the hematopoietic cells are hematopoietic stem cells (HSCs), CD34+ progenitor cells, in particular peripheral blood CD34+ cells, very early progenitor CD34+ cells, B-cell CD19+ progenitors, myeloid progenitor CD13+ cells, T lymphocytes, B lymphocytes, monocytes, dendritic cells, cancer B cells in particular B-cell chronic lymphocytic leukemia (BCLL) cells and marginal zone lymphoma (MZL) B cells, or thymocytes.

As known from the skilled person, many hematopoietic cells are produced from bone marrow hematopoietic stem cells.

In some embodiments, a hematopoietic cell is a hematopoietic stem cell (HSC), which are cells able to replenish all blood cell types and to self-renew. Hematopoietic stem cells may be in particular defined as cells that keep the levels of myeloid, T cells, and B cells at robustly detectable levels (typically more than 1% of peripheral blood cells) for 16 weeks when injected into the circulation of a recipient mouse with a depleted hematopoietic system (Schroeder (2010) Cell Stem Cell 6:203-207).

In some embodiments, the hematopoietic cell is a “CD34+ progenitor cell,” which is a heterogeneous cell population that includes a subpopulation of HSCs, pluripotent stem cells and cells in the early stages of lineage commitment. CD34+ progenitor cells continuously migrate to and from the bone marrow in normal adult animals. They can differentiate to produce all hematopoietic cell lineages found in the circulation. In some embodiments, the hematopoietic cell is a very early progenitor CD34+ cell which is a subgroup of CD34+ progenitor cells enriched from HSCs.

In some embodiments, the hematopoietic cell is a “peripheral blood CD34+ cell”, which is a CD34+ cell present in the blood.

In some embodiments, the hematopoietic cell is a B cell CD19+ progenitor, which is a population of B-lineage cells that express cell surface CD10, CD34, and CD19.

In some embodiments, the hematopoietic cell is a myeloid progenitor CD13+ cells, which is a population of myeloid lineage cells that express cell surface CD34 and CD13, and in some cases, also CD33.

In some embodiments, the target cell is selected from the group consisting of myeloid-lymphoid balanced hematopoietic lineage cells, myeloid-biased hematopoietic lineage cells, lymphoid-biased hematopoietic lineage cells, a platelet-biased hematopoietic lineage cells, a platelet-myeloid-biased hematopoietic lineage cells, a long-term repopulating hematopoietic lineage cells, an intermediate-term repopulating hematopoietic lineage cells, or a short-term repopulating hematopoietic lineage cells. In some embodiments, the target cell is selected from monocytes, macrophages, neutrophils, basophils, eosinophils, erythrocytes, megakaryocytes and platelets. In some embodiments, the target cell is selected from T cells, B cells, natural killer (NK) cells and innate lymphoid cells.

In some embodiments the target cell is an effector cell, e.g., a cell of the immune system that expresses one or more Fc receptors and mediates one or more effector functions. In some embodiments, a target cell may include one or more of a monocyte, macrophage, neutrophil, dendritic cell, eosinophil, mast cell, platelet, large granular lymphocyte, Langerhans' cell, natural killer (NK) cell, T lymphocyte (e.g., T cell), a Gamma delta T cell, B lymphocyte (e.g., B cell) and may be from any organism including humans, mice, rats, rabbits, and monkeys.

In some embodiment, the hematopoietic cell is a T cell. In some embodiments, the T cell is a naïve T cell. In some embodiments, the T cell is a memory T cell.

In some embodiments, the hematopoietic cell is a B cell. In some embodiments, the target cell is a resting B cell, such as a naive or a memory B cell. In some embodiments, the target cell is a cancer B cell, such as a B-cell chronic lymphocytic leukemia (BCLL) cell or a marginal zone lymphoma (MZL) B cell.

In some embodiments, the target cell is a thymocyte. In some embodiments, the target cell is a natural killer (NK) cell. In some embodiments, the thymocyte expresses CD4 or CD8. In some embodiments, the thymocyte does not express CD4 or CD8. In some embodiments, the natural killer (NK) cell is a cell that expresses CD56.

In some embodiments, the target cell is a CD3+ T cell, a CD4+ T cell, or a CD8+ T cell.

In some embodiments, the target cell is an antigen presenting cell, an MHC class II+ cell, a professional antigen presenting cell, an atypical antigen presenting cell, a macrophage, a dendritic cell, a myeloid dendritic cell, a plasmacyteoid dendritic cell, a CD11c+ cell, a CD11b+ cell, or a B cell.

In some embodiments, the binding domain (e.g. sdAb) variable domain binds a cell surface molecule or antigen. In some embodiments, the cell surface molecule is ASGR1, ASGR2, TM4SF5, CD3, CD8, CD4, CD7, or low density lipoprotein receptor (LDL-R). In some embodiments, the cell surface molecule is ASGR1. In some embodiments, the cell surface molecule is ASGR2. In some embodiments, the cell surface molecule is TM4SF5. In some embodiments, the cell surface molecule is CD3. In some embodiments, the cell surface molecule is CD8. In some embodiments, the cell surface molecule is CD4. In some embodiments, the cell surface molecule is LDL-R. In some embodiments, the cell surface molecule is ASCT2, CD105, CD110, CD117, CD133, CD146, CD164, CD34, CD46, CD49f, CD90, EPCR, or ITGA3.

In some embodiments, the retargeted attachment protein comprises the paramyxovirus envelope attachment protein (e.g., G protein or functionally active variant or biologically active portion thereof) linked directly to the binding domain and/or variable domain thereof. In some embodiments, the targeted envelope protein is a fusion protein that has the following structure: (N′-single domain antibody-C′)—(C′-G protein-N′).

In some embodiments, the retargeted attachment protein comprises the paramyxovirus envelope attachment protein (e.g., G protein or functionally active variant or biologically active portion thereof) linked indirectly via a linker to the binding domain and/or variable domain thereof. In some embodiments, the linker is a peptide linker. In some embodiments, the linker is a chemical linker.

In some embodiments, the linker is a peptide linker and the targeted envelope protein is a fusion protein containing the paramyxovirus envelope attachment protein (e.g., G protein or functionally active variant or biologically active portion thereof) linked via a peptide linker to the sdAb variable domain. In some embodiments, the targeted envelope protein is a fusion protein that has the following structure: (N′-single domain antibody-C′)-Linker-(C′-G protein-N′).

In some embodiments, the linker is a polypeptide linker. The polypeptide linker can be a flexible linker or a rigid linker or a combination of both. In some aspects, the linker is a short, medium or long linker. In some embodiments, the peptide linker is up to 65 amino acids in length. In some embodiments, the peptide linker comprises from or from about 2 to 65 amino acids, 2 to 60 amino acids, 2 to 56 amino acids, 2 to 52 amino acids, 2 to 48 amino acids, 2 to 44 amino acids, 2 to 40 amino acids, 2 to 36 amino acids, 2 to 32 amino acids, 2 to 28 amino acids, 2 to 24 amino acids, 2 to 20 amino acids, 2 to 18 amino acids, 2 to 14 amino acids, 2 to 12 amino acids, 2 to 10 amino acids, 2 to 8 amino acids, 2 to 6 amino acids, 6 to 65 amino acids, 6 to 60 amino acids, 6 to 56 amino acids, 6 to 52 amino acids, 6 to 48 amino acids, 6 to 44 amino acids, 6 to 40 amino acids, 6 to 36 amino acids, 6 to 32 amino acids, 6 to 28 amino acids, 6 to 24 amino acids, 6 to 20 amino acids, 6 to 18 amino acids, 6 to 14 amino acids, 6 to 12 amino acids, 6 to 10 amino acids, 6 to 8 amino acids, 8 to 65 amino acids, 8 to 60 amino acids, 8 to 56 amino acids, 8 to 52 amino acids, 8 to 48 amino acids, 8 to 44 amino acids, 8 to 40 amino acids, 8 to 36 amino acids, 8 to 32 amino acids, 8 to 28 amino acids, 8 to 24 amino acids, 8 to 20 amino acids, 8 to 18 amino acids, 8 to 14 amino acids, 8 to 12 amino acids, 8 to 10 amino acids, 10 to 65 amino acids, 10 to 60 amino acids, 10 to 56 amino acids, 10 to 52 amino acids, 10 to 48 amino acids, 10 to 44 amino acids, 10 to 40 amino acids, 10 to 36 amino acids, 10 to 32 amino acids, 10 to 28 amino acids, 10 to 24 amino acids, 10 to 20 amino acids, 10 to 18 amino acids, 10 to 14 amino acids, 10 to 12 amino acids, 12 to 65 amino acids, 12 to 60 amino acids, 12 to 56 amino acids, 12 to 52 amino acids, 12 to 48 amino acids, 12 to 44 amino acids, 12 to 40 amino acids, 12 to 36 amino acids, 12 to 32 amino acids, 12 to 28 amino acids, 12 to 24 amino acids, 12 to 20 amino acids, 12 to 18 amino acids, 12 to 14 amino acids, 14 to 65 amino acids, 14 to 60 amino acids, 14 to 56 amino acids, 14 to 52 amino acids, 14 to 48 amino acids, 14 to 44 amino acids, 14 to 40 amino acids, 14 to 36 amino acids, 14 to 32 amino acids, 14 to 28 amino acids, 14 to 24 amino acids, 14 to 20 amino acids, 14 to 18 amino acids, 18 to 65 amino acids, 18 to 60 amino acids, 18 to 56 amino acids, 18 to 52 amino acids, 18 to 48 amino acids, 18 to 44 amino acids, 18 to 40 amino acids, 18 to 36 amino acids, 18 to 32 amino acids, 18 to 28 amino acids, 18 to 24 amino acids, 18 to 20 amino acids, 20 to 65 amino acids, 20 to 60 amino acids, 20 to 56 amino acids, 20 to 52 amino acids, 20 to 48 amino acids, 20 to 44 amino acids, 20 to 40 amino acids, 20 to 36 amino acids, 20 to 32 amino acids, 20 to 28 amino acids, 20 to 26 amino acids, 20 to 24 amino acids, 24 to 65 amino acids, 24 to 60 amino acids, 24 to 56 amino acids, 24 to 52 amino acids, 24 to 48 amino acids, 24 to 44 amino acids, 24 to 40 amino acids, 24 to 36 amino acids, 24 to 32 amino acids, 24 to 30 amino acids, 24 to 28 amino acids, 28 to 65 amino acids, 28 to 60 amino acids, 28 to 56 amino acids, 28 to 52 amino acids, 28 to 48 amino acids, 28 to 44 amino acids, 28 to 40 amino acids, 28 to 36 amino acids, 28 to 34 amino acids, 28 to 32 amino acids, 32 to 65 amino acids, 32 to 60 amino acids, 32 to 56 amino acids, 32 to 52 amino acids, 32 to 48 amino acids, 32 to 44 amino acids, 32 to 40 amino acids, 32 to 38 amino acids, 32 to 36 amino acids, 36 to 65 amino acids, 36 to 60 amino acids, 36 to 56 amino acids, 36 to 52 amino acids, 36 to 48 amino acids, 36 to 44 amino acids, 36 to 40 amino acids, 40 to 65 amino acids, 40 to 60 amino acids, 40 to 56 amino acids, 40 to 52 amino acids, 40 to 48 amino acids, 40 to 44 amino acids, 44 to 65 amino acids, 44 to 60 amino acids, 44 to 56 amino acids, 44 to 52 amino acids, 44 to 48 amino acids, 48 to 65 amino acids, 48 to 60 amino acids, 48 to 56 amino acids, 48 to 52 amino acids, 50 to 65 amino acids, 50 to 60 amino acids, 50 to 56 amino acids, 50 to 52 amino acids, 54 to 65 amino acids, 54 to 60 amino acids, 54 to 56 amino acids, 58 to 65 amino acids, 58 to 60 amino acids, or 60 to 65 amino acids. In some embodiments, the peptide linker is a polypeptide that is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 amino acids in length.

The linkers can be naturally-occurring, synthetic or a combination of both. Natural linkers can be either flexible or constrained (e.g., structured) and can be very diverse in amino acid sequence and composition. Their degree of resistance to proteolysis depends on which proteins they originate from and which biological environment these proteins are facing in nature (extracellular, intracellular, prokaryotic, eukaryotic, etc.). In some embodiments, the linker is a linker based on peptides found in human proteins. Examples of natural linkers are: (i) KES GSVS SEQL AQFRSLD (see Bird et al. (1988) Science, 242, 423-426), (ii) sequences corresponding to the hinge domain of immunoglobulins devoid of light chains (see Hamers-Casterman et al. (1993) Nature, 363, 446-448 and PCT International Publication No: WO 096/34103). Examples of linkers for use with anti-albumin domain antibodies (e.g., human, humanized, camelized human or Camelid VHH domain antibodies) are EPKIPQPQPKPQPQPQPQPKPQPKPEPECTCPKCP and TNEVCKCPKCP. Other linkers derived from human and camelid hinges are disclosed in EPO656946, incorporated herein by reference. The hinge derived linkers can have variable lengths, for example from 0 to about 50 amino acids, including 1, 2, 3, 4, 5, 6, 7, 8, 9, 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 amino acids.

Particularly suitable linker polypeptides predominantly include amino acid residues selected from Glycine (Gly), Serine (Ser), Alanine (Ala), and Threonine (Thr). For example, the linker may contain at least 75% (calculated on the basis of the total number of residues present in the peptide linker), such as at least 80%, at least 85%, or at least 90% of amino acid residues selected from Gly, Ser, Ala, and Thr. The linker may also consist of Gly, Ser, Ala and/or Thr residues only. In some embodiments, the linker contains 1-25 glycine residues, 5-20 glycine residues, 5-15 glycine residues, or 8-12 glycine residues. In some aspects, suitable peptide linkers typically contain at least 50% glycine residues, such as at least 75% glycine residues. In some embodiments, a peptide linker comprises glycine residues only. In some embodiments, a peptide linker comprises glycine and serine residues only.

In particular embodiments, the linker is a flexible peptide linker. Flexible linkers are designed to adopt no stable secondary structure when connecting two polypeptide moieties, thus allowing a range of conformations in the fusion protein. These linkers are preferably hydrophilic in nature to prevent these from interacting with one or both fused polypeptides. Usually small polar residues such as glycine and serine are prevalent in those linkers in order to increase the flexible and hydrophilic characteristics of the peptide backbone, respectively. The length of these linkers is variable and best determined either empirically or with the aid of 3D computing approaches. In general, a preferred linker length will be the smallest compatible with good expression, good solubility and. full recovery of the native functions and structures of interest. Because of their flexible characteristics, flexible linkers may constitute good substrates for endogenous proteases. In general, unless it is a desirable feature flexible linkers are devoid of amino acids such as charged amino acids or large hydrophobic/aromatic which are readily recognized by endogenous proteases with broad substrate specificity.

In some embodiments, these linkers are composed predominately of the amino acids Glycine and Serine, denoted as GS-linkers herein. In some such embodiments, the linker is 1-20 amino acids, such as 1-20 amino acids predominantly composed of glycine. In some embodiments, the linker is 1-20 amino acids, such as 1-20 amino acids predominantly composed of glycine and serine. In some embodiments, the linker is a flexible peptide linker containing amino acids Glycine and Serine, referred to as GS-linkers. In some embodiments, the peptide linker includes the sequences GS, GGS, GGGGS, GGGGGS or combinations thereof. In some embodiments, the polypeptide linker has the sequence (GGS)n, wherein n is 1 to 10. In some embodiments, the polypeptide linker has the sequence (GGGGS)n, wherein n is 1 to 10. In some embodiments, the polypeptide linker has the sequence (GGGGGS)n, wherein n is 1 to 6. In some embodiments, the polypeptide linker has or comprises the amino acid sequence of SEQ ID NO: 405 (GGGGSGGGGSGGGGS).

In some cases, it may be desirable to provide some rigidity into the peptide linker. This may be accomplished by including proline residues in the amino acid sequence of the peptide linker. Thus, in some embodiments, a linker comprises at least one proline residue in the amino acid sequence of the peptide linker. For example, a peptide linker can have an amino acid sequence wherein at least 25% (e.g., at least 50% or at least 75%) of the amino acid residues are proline residues. In one particular embodiment, the peptide linker comprises proline residues only.

In some aspects, a peptide linker comprises at least one cysteine residue, such as one cysteine residue. For example, in some embodiments, a linker comprises at least one cysteine residue and amino acid residues selected from the group consisting of Gly, Ser, Ala, and Thr. In some such embodiments, a linker comprises glycine residues and cysteine residues, such as glycine residues and cysteine residues only. Typically, only one cysteine residue will be included per peptide linker. One example of a specific linker comprising a cysteine residue includes a peptide linker having the amino acid sequence Glym-Cys-Glyn, wherein n and m are each integers from 1-12, e.g., from 3-9, from 4-8, or from 4-7.

In some embodiments, the linker of the fusion protein is a structured linker. Constrained and/or structured linkers are designed to adopt a stable secondary structure when connecting two polypeptide moieties, thus restricting the range of conformations in the fusion protein. Such linkers usually adopt a helical structure spanning several turns. Again the length of these linkers is variable and best determined either empirically or with the aid of computing approaches. In some embodiments, constrained and/or structured linkers maintain the longest distance between each polypeptide of the fusion. This is particularly relevant when both polypeptides have a tendency to form hetero-aggregates. By virtue of their structure, constrained linkers can also be more resistant to proteolytic degradation, thereby offering an advantage when injected in vivo. Examples of constrained linkers are cited in PCT International Publications No: WO 00/24884 (e.g., SSSASASSA, GSPGSPG, or ATTTGSSPGPT), U.S. Pat. No. 6,132,992 (e.g., helical peptide linkers).

In particular embodiments, the structured linker contains the sequence (AP)n or (EAAAK)n, wherein n is 2 to 20, preferably 4 to 10, including but not limited to, AS-(AP)n-GT or AS-(EAAAK)n-GT, wherein n is 2 to 20, such as 2, 3, 4, 5, 6, 7, 8, 9, 10, 11, 12, 13, 14 or 15. In other embodiments, the linker comprises the sequences (GGGGA)n, (PGGGS)n, (AGGGS)n or GGS-(EGKSSGSGSESKST)n-GGS, wherein n is 2 to 20, (ADAAP)n (wherein n is 2 to 20), (ADAAP)n-G (wherein n is 2 to 20), (GEPQG)n (wherein n is 2 to 20), (GEPQG)n-G (wherein n is 2 to 20), (AGGEP)n (wherein n is 2 to 20), (AGGEP)n-G (wherein n is 2 to 20), (AGSEP)n (wherein n is 2 to 20), (AGSEP)n-G (wherein n is 2 to 20), (GGGEQ)n (wherein n is 2 to 20), (GGGEQ)n-G (wherein n is 2 to 20). In some embodiments, the linker is SSSASASSA, GSPGSPG, ATTTGSSPGPT, ADAAPADAAPG, GEPQGGEPQGG, AGGEPAGGEPG, AGSEPAGSEPG, or GGGEQGGGEQG.

In some embodiments, the polypeptide linker has or comprises the amino acid sequence of SEQ ID NO: 589 (AHHSED). In some embodiments, the polypeptide linker has or comprises the amino acid sequence of SEQ ID NO: 590 (EPKTPKPQPQPQPQPQPNPTTE).

In some embodiments, the retargeted attachment protein comprising a binding domain, a first binding domain, and/or a second binding domain linked to at least one paramyxovirus envelope attachment may comprise an engineered binding domain, such as an artificially generated binding domain. The binding domain may comprise a nanobody, a DARPin, an Aptamer, an Affimer, an Affibody, a Knottin, an Avimer, a Monobody, an Anticalin, a Fynomer. Any engineered binding domain known in art and suitable for the present invention can be used, for example any such binding domain described in Olaleye et al. Biomolecules. 2021 December; 11 (12): 1791.

### a. T Cell Binding Domains

In some embodiments, the lipid particles disclosed herein comprise one or more retargeted attachment proteins, each independently comprising (i) a paramyxovirus envelope attachment protein; and (ii) a targeting moiety directed to a target molecule expressed on the surface of a target cell. In some embodiments, the targeting moiety is a T cell binding domain, e.g., a T cell binding agent, such as any of those disclosed herein.

The lipid particles disclosed herein include, in some embodiments, one or more T cell binding domains (e.g., a T cell binding agent) that target the viral vector to a T cell. In some embodiments, the T cell binding agent binds to a molecule expressed on the surface of the T cell. The cell surface molecule may be a receptor, coreceptor, or a GPI-anchored protein. In some embodiments, the T cell binding agent binds CD3, CD4 or CD8.

In some embodiments, a T cell binding agent may be fused to or incorporated in a protein fusogen or lipid particle envelope attachment protein (e.g., a retargeted attachment protein). In some embodiments, a T cell binding agent may be incorporated into the viral envelope via fusion with a transmembrane domain. In some embodiments, the T cell binding agent targets the lipid particle to a T cell.

In particular embodiments, a T cell binding agent may be fused to or incorporated in a protein fusogen or attachment protein, thereby retargeting the lipid particle to a T cell. In some embodiments, for re-targeting the T cell binding agent is fused to a protein fusogen or envelope attachment protein that is mutated to reduce binding for the native binding partner of the fusogen or viral envelope protein. In some embodiments, the fusogen is or contains a mutant G protein or a biologically active portion thereof that is a mutant of wild-type NiV-G and exhibits reduced binding to one or both of the native binding partners Ephrin B2 or Ephrin B3, including any as described above. Thus, in some aspects, a fusogen can be retargeted to display altered tropism. In some embodiments, the binding confers re-targeted binding compared to the binding of a wild-type surface glycoprotein protein in which a new or different binding activity is conferred. In particular embodiments, the binding confers re-targeted binding compared to the binding of a wild-type G protein in which a new or different binding activity is conferred. In some embodiments the fusogen is randomly mutated. In some embodiments the fusogen is rationally mutated. In some embodiments the fusogen is subjected to directed evolution. In some embodiments the fusogen is truncated and only a subset of the peptide is used in the viral vector. In some embodiments, amino acid residues in the measles hemagglutinin protein may be mutated to alter the binding properties of the protein, redirecting fusion (doi: 10.1038/nbt942, Molecular Therapy vol. 16 no. 8, 1427-1436 August 2008, doi: 10.1038/nbt1060, DOI: 10.1128/JVI.76.7.3558-3563.2002, DOI: 10.1128/JVI.75.17.8016-8020.2001, doi: 10.1073pnas.0604993103).

In some embodiments, protein fusogens may be re-targeted by covalently conjugating a T cell binding agent to the attachment protein. In some embodiments, the fusogen and T cell binding agent are covalently conjugated by expression of a chimeric protein comprising the fusogen linked to the T cell binding agent (e.g., retargeted attachment protein). The T cell binding agent can include any targeting protein able to confer specific binding to a target molecule expressed on the surface of a T cell. In some embodiments, a targeting protein can also include an antibody or an antigen-binding fragment thereof (e.g., Fab, Fab′, F(ab′) 2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). In some embodiments, the T cell binding agent is an antibody or antigen binding fragment thereof. In some embodiments, the fusion protein can be engineered to bind the Fc region of an antibody that targets an antigen on a target cell, redirecting the fusion activity towards cells that display the antibody's target (DOI: 10.1128/JVI.75.17.8016-8020.2001, doi: 10.1038/nm1192). In some embodiments, altered and non-altered fusogens may be displayed on the same retroviral vector or VLP (doi: 10.1016/j.biomaterials.2014.01.051).

In some embodiments, a single-chain variable fragment (scFv) can be conjugated to fusogens to redirect fusion activity towards T cells that display the scFv binding target (doi: 10.1038/nbt1060, DOI 10.1182/blood-2012-11-468579, doi: 10.1038/nmeth.1514, doi: 10.1006/mthe.2002.0550, HUMAN GENE THERAPY 11:817-826, doi: 10.1038/nbt942, doi: 10.1371/journal.pone.0026381, DOI 10.1186/s12896-015-0142-z). In some embodiments, designed ankyrin repeat proteins (DARPin) can be conjugated to fusogens to redirect fusion activity towards T cells that display the DARPin binding target (doi: 10.1038/mt.2013.16, doi: 10.1038/mt.2010.298, doi: 10.4049/jimmunol.1500956), as well as combinations of different DARPins (doi: 10.1038/mto.2016.3). In some embodiments, a single domain antibody (e.g., a VHH) can be conjugated to fusogens to redirect fusion activity towards T cells that display the sdAb binding target. In some embodiments, receptor ligands and antigens can be conjugated to fusogens to redirect fusion activity towards T cells that display the target receptor (DOI: 10.1089/hgtb.2012.054, DOI: 10.1128/JVI.76.7.3558-3563.2002).

### i. CD3 Binding Agents

In some embodiments, the lipid particles disclosed herein comprise one or more retargeted attachment proteins, each independently comprising (i) a paramyxovirus envelope attachment protein; and (ii) a targeting moiety directed to a target molecule expressed on the surface of a target cell, wherein the target molecule is CD3. In some embodiments, the targeting moiety is a CD3 binding domain, e.g., a CD3 binding agent, such as any of those disclosed herein.

The lipid particles disclosed herein include, in some embodiments, one or more CD3 binding agents. For example, a CD3 binding agent may be fused to or incorporated in a retargeted attachment protein. In another embodiment, a CD3 binding agent may be incorporated into the lipid particle envelope via fusion with a transmembrane domain.

Exemplary CD3 binding agents include antibodies and fragments thereof (e.g., scFv, VHH) that bind to CD3. Such antibodies may be derived from any species, and may be for example, mouse, rabbit, human, humanized, or camelid antibodies.

Exemplary antibodies include OKT3, CRIS-7, I2C, blinatumomab, catumaxomab, muromonab-CD3, A-319, AFM11, AMG 199, AMG 211, AMG 424, AMG 427, AMG 562, AMG 564, APVO436, CC-93269, ERY974, GBR1302, GEM333, GEM2PSCA, GNC-035, HPN424, IGM-2323, JNJ-63709178, JNJ-63898081, JNJ-75348780, JNJ-78306358, M701, M802, MGD007, MOR209/ES414, PF-06671008, REGN5459, RO7283420, SAR442257, SAR443216, TNB-383B, TNB-486, TNB-585, Y150, acapatamab, cevostamab, cibisatamab, duvortuxizumab, eluvixtamab, emerfetamab, etevritamab, glofitamab, gresonitamab, obrindatamab, pavurutamab, plamotamab, solitomab, tarlatamab, tepoditamab, tidutamab, vibecotamab, vixtimotamab, alnuctamab, dafsolimab setaritox, pacanalotamab, pasotuxizumab, runimotamab, nivatrotamab, elranatamab, ertumaxomab, flotetuzumab, odronextamab, talquetamab, teclistamab, visilizumab, epcoritamab, otelixizumab, 3F₈BiAb, CCW702, DKTK CC-1, EMB-06, GEN1044, GEN1047, GTB-3550, HPN217, IMC-C103C, NVG-111, REGN4018, REGN4336, REGN5458, A-2019, A-337, ABP-100, AFM15, AFM21, AMG 701, APVO425, CLN-049, Dow2, EM801, Ektomab, FBTA05, GBR1342, GBR1372, GSK3537142, HBM7020, HLX31, IGM-2644, MG1122, MGD015, ND003, ND007, PF-07062119, RO7293583, STA551, TT19, ZW38; and anti-CD3 antibodies disclosed in U.S. Pat. Nos. 4,361,549, 7,728,114, 9,657,102, 9,587,021, and 11007267; US Patent Application Nos. US20120269826, US20180057597, and US20180112000; and PCT Application Nos. WO2005118635, WO2011050106, WO2012162067, WO2014047231, WO2016116626, WO2016180721, and WO2016204966. Other exemplary binding agents include designed ankyrin repeat proteins (DARPins) and binding agents based on fibronectin type III (Fn3) scaffolds.

In some embodiments, the CD3 binding agent comprises a heavy chain variable (VH) region comprising a CDR-H1, a CDRH-2, and a CDR-H3 comprising the amino acid sequence set forth in SEQ ID NO: 474, 475, and 476 respectively; and a light chain variable region comprising a CDR-L1, a CDR-L2, and a CDR-L3 comprising the amino acid sequence set forth in SEQ ID NO:477, 478, and 479, respectively. In some embodiments, the CD3 binding agent comprises a VH region comprising an amino acid sequence having at least about 90% sequence identity to the amino acid sequence set forth in SEQ ID NO: 480, and a VL region comprising an amino acid sequence having at least about 90% sequence identity to the amino acid sequence set forth in SEQ ID NO:481. In some embodiments, the CD3 binding agent comprises a VH region comprising the amino acid sequence set forth in SEQ ID NO:480, and a VL region comprising the amino acid sequence set forth in SEQ ID NO:481. In some embodiments, the CD3 binding agent is an scFv. In some embodiments, the CD3 binding agent comprises the amino acid sequence set forth in SEQ ID NO:482. In some embodiments, the CD3 binding agent is OKT3.

In some embodiments, the CD3 binding agent is activating (e.g., the CD3 binding agent activates T cells). In some embodiments, the CD3 binding agent is non-activating (e.g., it does not activate T cells).

In some embodiments, a CD3 binding agent comprises a humanized antibody molecule, intact IgA, IgG, IgE or IgM antibody; bi- or multi-specific antibody (e.g., Zybodies®, etc.); antibody fragments such as Fab fragments, Fab′ fragments, F(ab′) 2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); camelid antibodies; masked antibodies (e.g., Probodies®); Small Modular ImmunoPharmaceuticals (“SMIPsTM”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-Bodies®; Affibodies®; TrimerX®; MicroProteins; Fynomers®, Centyrins®; and KALBITOR®s.

In some embodiments, the CD3 binding agent is a peptide. In some embodiments, the CD3 binding agent is an antibody, such as a single-chain variable fragment (scFv). In some embodiments, the CD3 binding agent is an antibody, such as a single domain antibody. In some embodiments, the antibody can be human or humanized. In some embodiments, the CD3 binding agent is a VHH. In some embodiments, the antibody or portion thereof is naturally occurring. In some embodiments, the antibody or portion thereof is synthetic.

In some embodiments, the antibody can be generated from phage display libraries to have specificity for a desired target ligand. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, a library of human single domain antibodies is synthetically generated by introducing diversity into one or more scaffolds.

In some embodiments, the C-terminus of the CD3 binding agent is attached to the C-terminus of the G protein (e.g., fusogen) or biologically active portion thereof. In some embodiments, the N-terminus of the CD3 binding agent is exposed on the exterior surface of the lipid bilayer.

In some embodiments, the CD3 binding agent is the only surface displayed non-viral sequence of the viral vector. In some embodiments, the CD3 binding agent is the only membrane bound non-viral sequence of the viral vector. In some embodiments, the viral vector does not contain a molecule that engages or stimulates T cells other than the CD3 binding agent. In some embodiments, the viral vector contains a non-activating CD3 binding agent.

In some embodiments, viral vectors may display CD3 binding agents that are not conjugated to protein fusogens in order to redirect the fusion activity towards a cell that is bound by the targeting moiety, or to affect homing.

### ii. CD4 Binding Agents

In some embodiments, the lipid particles disclosed herein comprise one or more retargeted attachment proteins, each independently comprising (i) a paramyxovirus envelope attachment protein; and (ii) a targeting moiety directed to a target molecule expressed on the surface of a target cell, wherein the target molecule is CD4. In some embodiments, the targeting moiety is a CD4 binding domain, e.g., a CD4 binding agent, such as any of those disclosed herein.

The lipid particles disclosed herein include, in some embodiments, one or more CD4 binding agents. For example, a CD4 binding agent may be fused to or incorporated in a protein fusogen or attachment protein. In another embodiment, a CD4 binding agent may be incorporated into the lipid particle envelope via fusion with a transmembrane domain.

In some of any of the provided embodiments, the CD4 binding agent is exposed on the surface of the lipid particle. In some embodiments, the CD4 binding agent is fused to a transmembrane domain incorporated in the lipid particle envelope.

Exemplary CD4 binding agents include antibodies and fragments thereof (e.g., scFv, VHH) that bind to CD4. Such antibodies may be derived from any species, and may be for example, mouse, rabbit, human, humanized, or camelid antibodies. Exemplary antibodies include ibalizumab, zanolimumab, tregalizumab, priliximab, cedelizumab, clenoliximab, keliximab, and anti-CD4 antibodies disclosed in WO2002102853, WO2004083247, WO2004067554, WO2007109052, WO2008134046, WO2010074266, WO2012113348, WO2013188870, WO2017104735, WO2018035001, WO2018170096, WO2019203497, WO2019236684, WO2020228824, U.S. Pat. Nos. 5,871,732, 7,338,658, 7,722,873, 8,399,621, 8,911,728, 9,005,963, U.S. Pat. Nos. 9,587,022, 9,745,552, U.S. provisional application No. 63/326,269, U.S. provisional application No. 63/341,681; as well as antibodies B486A1, RPA-T4, CE9.1 (Novus Biologicals); GK1.5, RM4-5, RPA-T4, OKT4, 4SM95, S3.5, N1UG0 (ThermoFisher); GTX50984, ST0488, 10B5, EP204 (GeneTex); GK1.3, 5A8, 10C12, W3/25, 8A5, 13B8.2, 6G5 (Absolute Antibody); VIT4, M-T466, M-T321, REA623, (Miltenyi); MEM115, MT310 (Enzo Life Sciences); H129.19, 5B4, 6A17, 18-46, A-1, C-1, OX68 (Santa Cruz); EP204, D2E6M (Cell Signaling Technology). Other exemplary binding agents include designed ankyrin repeat proteins (DARPins) (e.g., the anti-CD4 DARPin disclosed in WO2017182585) and binding agents based on fibronectin type III (Fn3) scaffolds. Each of U.S. Pat. No. 9,005,963, U.S. provisional application No. 63/326,269, and U.S. provisional application No. 63/341,681 is incorporated by reference herein in its entirety.

In some embodiments, protein fusogens or attachment proteins may be re-targeted by mutating amino acid residues in a fusion protein or a targeting protein (e.g. the hemagglutinin (H) protein or G protein). In particular embodiments, the fusogen (e.g. G protein) is mutated to reduce binding for the native binding partner of the fusogen. In some embodiments, the fusogen is or contains a mutant G protein or a biologically active portion thereof that is a mutant of wild-type NiV-G and exhibits reduced binding to one or both of the native binding partners Ephrin B2 or Ephrin B3, including any as described above. Thus, in some aspects, a fusogen can be retargeted to display altered tropism. In some embodiments, the binding confers re-targeted binding compared to the binding of a wild-type surface glycoprotein protein in which a new or different binding activity is conferred. In particular embodiments, the binding confers re-targeted binding compared to the binding of a wild-type G protein in which a new or different binding activity is conferred. In some embodiments the fusogen is randomly mutated. In some embodiments the fusogen is rationally mutated. In some embodiments the fusogen is subjected to directed evolution. In some embodiments the fusogen is truncated and only a subset of the peptide is used in the viral vector. In some embodiments, amino acid residues in the measles hemagglutinin protein may be mutated to alter the binding properties of the protein, redirecting fusion (doi: 10.1038/nbt942, Molecular Therapy vol. 16 no. 8, 1427-1436 August 2008, doi: 10.1038/nbt1060, DOI: 10.1128/JVI.76.7.3558-3563.2002, DOI: 10.1128/JVI.75.17.8016-8020.2001, doi: 10.1073pnas.0604993103).

In some embodiments, protein fusogens may be re-targeted by covalently conjugating a CD4 binding agent to the fusion protein or attachment protein (e.g. retargeted attachment protein). In some embodiments, the fusogen and CD4 binding agent are covalently conjugated by expression of a chimeric protein comprising the fusogen linked to the CD4 binding agent (e.g. retargeted attachment protein). In some embodiments, a single-chain variable fragment (scFv) can be conjugated to fusogens to redirect fusion activity towards cells that display the scFv binding target (doi: 10.1038/nbt1060, DOI 10.1182/blood-2012-11-468579, doi: 10.1038/nmeth.1514, doi: 10.1006/mthe.2002.0550, HUMAN GENE THERAPY 11:817-826, doi: 10.1038/nbt942, doi: 10.1371/journal.pone.0026381, DOI 10.1186/s12896-015-0142-z). In some embodiments, designed ankyrin repeat proteins (DARPin) can be conjugated to fusogens to redirect fusion activity towards cells that display the DARPin binding target (doi: 10.1038/mt.2013.16, doi: 10.1038/mt.2010.298, doi: 10.4049/jimmunol.1500956), as well as combinations of different DARPins (doi: 10.1038/mto.2016.3). In some embodiments, receptor ligands and antigens can be conjugated to fusogens to redirect fusion activity towards cells that display the target receptor (DOI: 10.1089/hgtb.2012.054, DOI: 10.1128/JVI.76.7.3558-3563.2002). In some embodiments, a targeting protein can also include an antibody or an antigen-binding fragment thereof (e.g., Fab, Fab′, F (ab′) 2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). In some embodiments, protein fusogens may be re-targeted by non-covalently conjugating a CD4 binding agent to the fusion protein or targeting protein (e.g. retargeted attachment protein)). In some embodiments, the fusion protein can be engineered to bind the Fc region of an antibody that targets an antigen on a target cell, redirecting the fusion activity towards cells that display the antibody's target (DOI: 10.1128/JVI.75.17.8016-8020.2001, doi: 10.1038/nm1192). In some embodiments, altered and non-altered fusogens may be displayed on the same retroviral vector or VLP (doi: 10.1016/j.biomaterials.2014.01.051).

In some embodiments, a CD4 binding agent comprises a humanized antibody molecule, intact IgA, IgG, IgE or IgM antibody; bi- or multi-specific antibody (e.g., Zybodies®, etc.); antibody fragments such as Fab fragments, Fab′ fragments, F(ab′) 2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); camelid antibodies; masked antibodies (e.g., Probodies®); Small Modular ImmunoPharmaceuticals (“SMIPsTM”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-Bodies®; Affibodies®; TrimerX®; MicroProteins; Fynomers®, Centyrins®; and KALBITOR®s.

In some embodiments, the CD4 binding agent is a peptide. In some embodiments, the CD4 binding agent is an antibody, such as a single-chain variable fragment (scFv). In some embodiments, the CD4 binding agent is an antibody, such as a single domain antibody. In some embodiments, the antibody can be human or humanized. In some embodiments, the CD4 binding agent is a VHH. In some embodiments, the antibody or portion thereof is naturally occurring. In some embodiments, the antibody or portion thereof is synthetic.

In some embodiments, the antibody can be generated from phage display libraries to have specificity for a desired target ligand. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, a library of human single domain antibodies is synthetically generated by introducing diversity into one or more scaffolds.

In some embodiments, the C-terminus of the CD4 binding agent is attached to the C-terminus of the G protein (e.g., fusogen) or biologically active portion thereof. In some embodiments, the N-terminus of the CD4 binding agent is exposed on the exterior surface of the lipid bilayer.

In some embodiments, the CD4 binding agent is the only surface displayed non-viral sequence of the lipid particle. In some embodiments, the CD4 binding agent is the only membrane bound non-viral sequence of the lipid particle. In some embodiments, the lipid particle does not contain a molecule that engages or stimulates T cells other than the CD4 binding agent.

In some embodiments, lipid particles may display CD4 binding agents that are not conjugated to protein fusogens in order to redirect the fusion activity towards a cell that is bound by the targeting moiety, or to affect homing.

In some embodiments, a protein fusogen derived from a virus or organism that do not infect humans does not have a natural fusion targets in patients, and thus has high specificity.

In some of any of the provided embodiments, the CD4 binding agent is an anti-CD4 antibody or an antigen-binding fragment. In some of any of the provided embodiments, the anti-CD4 antibody or antigen-binding fragment is mouse, rabbit, human, or humanized. In some embodiments, the antigen-binding fragment is a single chain variable fragment (scFv). In some embodiments, the antigen-binding fragment is an anti-CD4 scFv.

In some embodiments, the anti-CD4 scFv comprises a CDR-H1, a CDR-H2, and a CDR-H3 comprising the amino acid sequence set forth in SEQ ID NO: 260, 261, and 262, respectively. In some embodiments, the anti-CD4 scFv comprises a CDR-L1, a CDR-L2, and a CDR-L3 comprising the amino acid sequence set forth in SEQ ID NO: 263, 264, and 265, respectively. In some embodiments, the anti-CD4 scFv comprises a CDR-H1, a CDR-H2, and a CDR-H3 comprising the amino acid sequence set forth in SEQ ID NO: 260, 261, and 262, respectively; and a CDR-L1, a CDR-L2, and a CDR-L3 comprising the amino acid sequence set forth in SEQ ID NO: 263, 264, and 265, respectively. In some embodiments, the anti-CD4 scFv comprises a CDR-H1, a CDR-H2, and a CDR-H3 comprising the amino acid sequence set forth in SEQ ID NO: 266, 267, and 268, respectively. In some embodiments, the anti-CD4 scFv comprises a CDR-L1, a CDR-L2, and a CDR-L3 comprising the amino acid sequence set forth in SEQ ID NO: 269, 270, and 265, respectively. In some embodiments, the anti-CD4 scFv comprises a CDR-H1, a CDR-H2, and a CDR-H3 comprising the amino acid sequence set forth in SEQ ID NO: 266, 267, and 268, respectively; and a CDR-L1, a CDR-L2, and a CDR-L3 comprising the amino acid sequence set forth in SEQ ID NO: 269, 270, and 265, respectively. In some embodiments, the anti-CD4 scFv comprises a CDR-H1, a CDR-H2, and a CDR-H3 comprising the amino acid sequence set forth in SEQ ID NO: 271, 272, and 268, respectively. In some embodiments, the anti-CD4 scFv comprises a CDR-L1, a CDR-L2, and a CDR-L3 comprising the amino acid sequence set forth in SEQ ID NO: 269, 270, and 265, respectively. In some embodiments, the anti-CD4 scFv comprises a CDR-H1, a CDR-H2, and a CDR-H3 comprising the amino acid sequence set forth in SEQ ID NO: 271, 272, and 268, respectively; and a CDR-L1, a CDR-L2, and a CDR-L3 comprising the amino acid sequence set forth in SEQ ID NO: 269, 270, and 265, respectively. In some embodiments, the anti-CD4 scFv comprises a heavy chain variable region (VH) comprising the amino acid sequence set forth in SEQ ID NO: 273. In some embodiments, the anti-CD4 scFv comprises a light chain variable region (VL) comprising the amino acid sequence set forth in SEQ ID NO:274. In some embodiments, the anti-CD4 scFv comprises a heavy chain variable region (VH) comprising the amino acid sequence set forth in SEQ ID NO: 273; and a light chain variable region (VL) comprising the amino acid sequence set forth in SEQ ID NO: 274. In some embodiments, the VH and VL are joined by a linker. In some embodiments, the linker comprises the amino acid sequence set forth in SEQ ID NO:275. In some embodiments, the anti-CD4 scFv comprises the amino acid sequence set forth in SEQ ID NO:276.

In some embodiments, the anti-CD4 scFv comprises a CDR-H1, a CDR-H2, and a CDR-H3 comprising the amino acid sequence set forth in SEQ ID NO: 277, 278, and 279, respectively. In some embodiments, the anti-CD4 scFv comprises a CDR-L1, a CDR-L2, and a CDR-L3 comprising the amino acid sequence set forth in SEQ ID NO: 280, 281, and 282, respectively. In some embodiments, the anti-CD4 scFv comprises a CDR-H1, a CDR-H2, and a CDR-H3 comprising the amino acid sequence set forth in SEQ ID NO: 277, 278, and 279, respectively; and a CDR-L1, a CDR-L2, and a CDR-L3 comprising the amino acid sequence set forth in SEQ ID NO: 280, 281, and 282, respectively. In some embodiments, the anti-CD4 scFv comprises a CDR-H1, a CDR-H2, and a CDR-H3 comprising the amino acid sequence set forth in SEQ ID NO: 283, 284, and 285, respectively. In some embodiments, the anti-CD4 scFv comprises a CDR-L1, a CDR-L2, and a CDR-L3 comprising the amino acid sequence set forth in SEQ ID NO: 286, 287, and 288, respectively. In some embodiments, the anti-CD4 scFv comprises a CDR-H1, a CDR-H2, and a CDR-H3 comprising the amino acid sequence set forth in SEQ ID NO: 283, 284, and 285, respectively; and a CDR-L1, a CDR-L2, and a CDR-L3 comprising the amino acid sequence set forth in SEQ ID NO: 286, 287, and 288, respectively. In some embodiments, the anti-CD4 scFv comprises a CDR-H1, a CDR-H2, and a CDR-H3 comprising the amino acid sequence set forth in SEQ ID NO: 289, 290, and 285, respectively. In some embodiments, the anti-CD4 scFv comprises a CDR-L1, a CDR-L2, and a CDR-L3 comprising the amino acid sequence set forth in SEQ ID NO: 286, 287, and 282, respectively. In some embodiments, the anti-CD4 scFv comprises a CDR-H1, a CDR-H2, and a CDR-H3 comprising the amino acid sequence set forth in SEQ ID NO: 289, 290, and 285, respectively; and a CDR-L1, a CDR-L2, and a CDR-L3 comprising the amino acid sequence set forth in SEQ ID NO: 286, 287, and 282, respectively. In some embodiments, the anti-CD4 scFv comprises a heavy chain variable region (VH) comprising the amino acid sequence set forth in SEQ ID NO: 291. In some embodiments, the anti-CD4 scFv comprises a light chain variable region (VL) comprising the amino acid sequence set forth in SEQ ID NO:292. In some embodiments, the anti-CD4 scFv comprises a heavy chain variable region (VH) comprising the amino acid sequence set forth in SEQ ID NO: 291; and a light chain variable region (VL) comprising the amino acid sequence set forth in SEQ ID NO: 292. In some embodiments, the VH and VL are joined by a linker. In some embodiments, the linker comprises the amino acid sequence set forth in SEQ ID NO:275. In some embodiments, the anti-CD4 scFv comprises the amino acid sequence set forth in SEQ ID NO:293.

In some embodiments, the anti-CD4 scFv comprises a CDR-H1, a CDR-H2, and a CDR-H3 comprising the amino acid sequence set forth in SEQ ID NO: 294, 295, and 296, respectively. In some embodiments, the anti-CD4 scFv comprises a CDR-L1, a CDR-L2, and a CDR-L3 comprising the amino acid sequence set forth in SEQ ID NO: 297, 298, and 299, respectively. In some embodiments, the anti-CD4 scFv comprises a CDR-H1, a CDR-H2, and a CDR-H3 comprising the amino acid sequence set forth in SEQ ID NO: 294, 295, and 296, respectively; and a CDR-L1, a CDR-L2, and a CDR-L3 comprising the amino acid sequence set forth in SEQ ID NO: 297, 298, and 299, respectively. In some embodiments, the anti-CD4 scFv comprises a CDR-H1, a CDR-H2, and a CDR-H3 comprising the amino acid sequence set forth in SEQ ID NO: 300, 301, 302, respectively. In some embodiments, the anti-CD4 scFv comprises a CDR-L1, a CDR-L2, and a CDR-L3 comprising the amino acid sequence set forth in SEQ ID NO: 303, 304, and 299, respectively. In some embodiments, the anti-CD4 scFv comprises a CDR-H1, a CDR-H2, and a CDR-H3 comprising the amino acid sequence set forth in SEQ ID NO: 300, 301, 302, respectively; and a CDR-L1, a CDR-L2, and a CDR-L3 comprising the amino acid sequence set forth in SEQ ID NO: 303, 304, and 299, respectively. In some embodiments, the anti-CD4 scFv comprises a CDR-H1, a CDR-H2, and a CDR-H3 comprising the amino acid sequence set forth in SEQ ID NO: 305, 306, 306, respectively. In some embodiments, the anti-CD4 scFv comprises a CDR-L1, a CDR-L2, and a CDR-L3 comprising the amino acid sequence set forth in SEQ ID NO: 303, 304, and 299, respectively. In some embodiments, the anti-CD4 scFv comprises a CDR-H1, a CDR-H2, and a CDR-H3 comprising the amino acid sequence set forth in SEQ ID NO: 305, 306, 302, respectively; and a CDR-L1, a CDR-L2, and a CDR-L3 comprising the amino acid sequence set forth in SEQ ID NO: 224, 225, and 172, respectively. In some embodiments, the anti-CD4 scFv comprises a heavy chain variable region (VH) comprising the amino acid sequence set forth in SEQ ID NO:307. In some embodiments, the anti-CD4 scFv comprises a light chain variable region (VL) comprising the amino acid sequence set forth in SEQ ID NO:308. In some embodiments, the anti-CD4 scFv comprises a heavy chain variable region (VH) comprising the amino acid sequence set forth in SEQ ID NO:307; and a light chain variable region (VL) comprising the amino acid sequence set forth in SEQ ID NO:308. In some embodiments, the VH and VL are joined by a linker. In some embodiments, the linker comprises the amino acid sequence set forth in SEQ ID NO:275. In some embodiments, the anti-CD4 scFv comprises the amino acid sequence set forth in SEQ ID NO:309.

In some embodiments, the anti-CD4 scFv comprises a CDR-H1, a CDR-H2, and a CDR-H3 comprising the amino acid sequence set forth in SEQ ID NO: 310, 311, and 312, respectively. In some embodiments, the anti-CD4 scFv comprises a CDR-L1, a CDR-L2, and a CDR-L3 comprising the amino acid sequence set forth in SEQ ID NO: 313, 314, and 315, respectively. In some embodiments, the anti-CD4 scFv comprises a CDR-H1, a CDR-H2, and a CDR-H3 comprising the amino acid sequence set forth in SEQ ID NO: 310, 311, and 312, respectively; and a CDR-L1, a CDR-L2, and a CDR-L3 comprising the amino acid sequence set forth in SEQ ID NO: 313, 314, and 315, respectively. In some embodiments, the anti-CD4 scFv comprises a CDR-H1, a CDR-H2, and a CDR-H3 comprising the amino acid sequence set forth in SEQ ID NO: 316, 317, 318, respectively. In some embodiments, the anti-CD4 scFv comprises a CDR-L1, a CDR-L2, and a CDR-L3 comprising the amino acid sequence set forth in SEQ ID NO: 319, 320, and 315, respectively. In some embodiments, the anti-CD4 scFv comprises a CDR-H1, a CDR-H2, and a CDR-H3 comprising the amino acid sequence set forth in SEQ ID NO: 316, 317, 318, respectively; and a CDR-L1, a CDR-L2, and a CDR-L3 comprising the amino acid sequence set forth in SEQ ID NO: 319, 320, and 315, respectively. In some embodiments, the anti-CD4 scFv comprises a CDR-H1, a CDR-H2, and a CDR-H3 comprising the amino acid sequence set forth in SEQ ID NO: 321, 322, 318, respectively. In some embodiments, the anti-CD4 scFv comprises a CDR-L1, a CDR-L2, and a CDR-L3 comprising the amino acid sequence set forth in SEQ ID NO: 319, 321, and 315, respectively. In some embodiments, the anti-CD4 scFv comprises a CDR-H1, a CDR-H2, and a CDR-H3 comprising the amino acid sequence set forth in SEQ ID NO: 321, 322, 318, respectively; and a CDR-L1, a CDR-L2, and a CDR-L3 comprising the amino acid sequence set forth in SEQ ID NO: 319, 320, and 323, respectively. In some embodiments, the anti-CD4 scFv comprises a heavy chain variable region (VH) comprising the amino acid sequence set forth in SEQ ID NO:323. In some embodiments, the anti-CD4 scFv comprises a light chain variable region (VL) comprising the amino acid sequence set forth in SEQ ID NO:324. In some embodiments, the anti-CD4 scFv comprises a heavy chain variable region (VH) comprising the amino acid sequence set forth in SEQ ID NO:323; and a light chain variable region (VL) comprising the amino acid sequence set forth in SEQ ID NO:324. In some embodiments, the VH and VL are joined by a linker. In some embodiments, the linker comprises the amino acid sequence set forth in SEQ ID NO:275. In some embodiments, the anti-CD4 scFv comprises the amino acid sequence set forth in SEQ ID NO:325.

In some embodiments, the anti-CD4 scFv comprises a CDR-H1, a CDR-H2, and a CDR-H3 comprising the amino acid sequence set forth in SEQ ID NO: 326, 327, and 328, respectively. In some embodiments, the anti-CD4 scFv comprises a CDR-L1, a CDR-L2, and a CDR-L3 comprising the amino acid sequence set forth in SEQ ID NO: 329, 330, and 331, respectively. In some embodiments, the anti-CD4 scFv comprises a CDR-H1, a CDR-H2, and a CDR-H3 comprising the amino acid sequence set forth in SEQ ID NO: 326, 327, and 328, respectively; and a CDR-L1, a CDR-L2, and a CDR-L3 comprising the amino acid sequence set forth in SEQ ID NO: 329, 330, and 331, respectively. In some embodiments, the anti-CD4 scFv comprises a CDR-H1, a CDR-H2, and a CDR-H3 comprising the amino acid sequence set forth in SEQ ID NO: 332, 333, and 334, respectively. In some embodiments, the anti-CD4 scFv comprises a CDR-L1, a CDR-L2, and a CDR-L3 comprising the amino acid sequence set forth in SEQ ID NO: 335, 336, and 331, respectively. In some embodiments, the anti-CD4 scFv comprises a CDR-H1, a CDR-H2, and a CDR-H3 comprising the amino acid sequence set forth in SEQ ID NO: 332, 333, and 334, respectively; and a CDR-L1, a CDR-L2, and a CDR-L3 comprising the amino acid sequence set forth in SEQ ID NO: 335, 336, and 331, respectively. In some embodiments, the anti-CD4 scFv comprises a CDR-H1, a CDR-H2, and a CDR-H3 comprising the amino acid sequence set forth in SEQ ID NO: 337, 338, and 334, respectively. In some embodiments, the anti-CD4 scFv comprises a CDR-L1, a CDR-L2, and a CDR-L3 comprising the amino acid sequence set forth in SEQ ID NO: 335, 336, and 331, respectively. In some embodiments, the anti-CD4 scFv comprises a CDR-H1, a CDR-H2, and a CDR-H3 comprising the amino acid sequence set forth in SEQ ID NO: 337, 338, and 334, respectively; and a CDR-L1, a CDR-L2, and a CDR-L3 comprising the amino acid sequence set forth in SEQ ID NO: 335, 336, and 331, respectively. In some embodiments, the anti-CD4 scFv comprises a heavy chain variable region (VH) comprising the amino acid sequence set forth in SEQ ID NO: 339. In some embodiments, the anti-CD4 scFv comprises a light chain variable region (VL) comprising the amino acid sequence set forth in SEQ ID NO:340. In some embodiments, the anti-CD4 scFv comprises a heavy chain variable region (VH) comprising the amino acid sequence set forth in SEQ ID NO: 339; and a light chain variable region (VL) comprising the amino acid sequence set forth in SEQ ID NO: 340. In some embodiments, the VH and VL are joined by a linker. In some embodiments, the linker comprises the amino acid sequence set forth in SEQ ID NO:275. In some embodiments, the anti-CD4 scFv comprises the amino acid sequence set forth in SEQ ID NO:341.

In some embodiments, the anti-CD4 scFv comprises a CDR-H1, a CDR-H2, and a CDR-H3 comprising the amino acid sequence set forth in SEQ ID NO: 342, 343, and 344, respectively. In some embodiments, the anti-CD4 scFv comprises a CDR-L1, a CDR-L2, and a CDR-L3 comprising the amino acid sequence set forth in SEQ ID NO: 345, 346, and 347, respectively. In some embodiments, the anti-CD4 scFv comprises a CDR-H1, a CDR-H2, and a CDR-H3 comprising the amino acid sequence set forth in SEQ ID NO: 342, 343, and 344, respectively; and a CDR-L1, a CDR-L2, and a CDR-L3 comprising the amino acid sequence set forth in SEQ ID NO: 345, 346, and 347, respectively. In some embodiments, the anti-CD4 scFv comprises a CDR-H1, a CDR-H2, and a CDR-H3 comprising the amino acid sequence set forth in SEQ ID NO: 348, 349, and 350, respectively. In some embodiments, the anti-CD4 scFv comprises a CDR-L1, a CDR-L2, and a CDR-L3 comprising the amino acid sequence set forth in SEQ ID NO: 351, 352, and 347, respectively. In some embodiments, the anti-CD4 scFv comprises a CDR-H1, a CDR-H2, and a CDR-H3 comprising the amino acid sequence set forth in SEQ ID NO: 242, 243, and 244, respectively; and a CDR-L1, a CDR-L2, and a CDR-L3 comprising the amino acid sequence set forth in SEQ ID NO: 245, 246, and 198, respectively. In some embodiments, the anti-CD4 scFv comprises a CDR-H1, a CDR-H2, and a CDR-H3 comprising the amino acid sequence set forth in SEQ ID NO: 353, 354, and 350, respectively. In some embodiments, the anti-CD4 scFv comprises a CDR-L1, a CDR-L2, and a CDR-L3 comprising the amino acid sequence set forth in SEQ ID NO: 351, 353, and 347, respectively. In some embodiments, the anti-CD4 scFv comprises a CDR-H1, a CDR-H2, and a CDR-H3 comprising the amino acid sequence set forth in SEQ ID NO: 353, 354, and 350, respectively; and a CDR-L1, a CDR-L2, and a CDR-L3 comprising the amino acid sequence set forth in SEQ ID NO: 351, 352, and 347, respectively. In some embodiments, the anti-CD4 scFv comprises a heavy chain variable region (VH) comprising the amino acid sequence set forth in SEQ ID NO: 355. In some embodiments, the anti-CD4 scFv comprises a light chain variable region (VL) comprising the amino acid sequence set forth in SEQ ID NO:356. In some embodiments, the anti-CD4 scFv comprises a heavy chain variable region (VH) comprising the amino acid sequence set forth in SEQ ID NO: 355; and a light chain variable region (VL) comprising the amino acid sequence set forth in SEQ ID NO: 356. In some embodiments, the VH and VL are joined by a linker. In some embodiments, the linker comprises the amino acid sequence set forth in SEQ ID NO:275. In some embodiments, the anti-CD4 scFv comprises the amino acid sequence set forth in SEQ ID NO:357.

In some embodiments, the anti-CD4 antibody or antigen-binding fragment is a single domain antibody. In some embodiments, the anti-CD4 antibody or antigen-binding fragment is a camelid (e.g. llama, alpaca, camel) anti-CD4 antibody or antigen-binding fragment (e.g. VHH). In some embodiments, the anti-CD4 antibody or antigen-binding fragment is an anti-CD4 VHH. In some embodiments, the anti-CD4 VHH comprises a CDR-H1, a CDR-H2, and a CDR-H3 comprising the amino acid sequence set forth in SEQ ID NO: 358, 359, and 360, respectively. In some embodiments, the anti-CD4 VHH comprises a CDR-H1, a CDR-H2, and a CDR-H3 comprising the amino acid sequence set forth in SEQ ID NO: 361, 362, and 363, respectively. In some embodiments, the anti-CD4 VHH comprises a CDR-H1, a CDR-H2, and a CDR-H3 comprising the amino acid sequence set forth in SEQ ID NO: 364, 365, and 363, respectively. In some embodiments, the anti-CD4 VHH comprises the amino acid sequence set forth in SEQ ID NO:366.

### iii. CD7 Binding Agents

In some embodiments, the lipid particles disclosed herein comprise one or more retargeted attachment proteins, each independently comprising (i) a paramyxovirus envelope attachment protein; and (ii) a targeting moiety directed to a target molecule expressed on the surface of a target cell, wherein the target molecule is CD7. In some embodiments, the targeting moiety is a CD7 binding domain, e.g., a CD7 binding agent, such as any of those disclosed herein.

The lipid particles disclosed herein include, in some embodiments, one or more CD7 binding agents. For example, a CD7 binding agent may be fused to or incorporated in a protein fusogen or attachment protein. In another embodiment, a CD7 binding agent may be incorporated into the lipid particle envelope via fusion with a transmembrane domain.

Exemplary CD7 binding agents include antibodies and fragments thereof (e.g., scFv, VHH) that bind to CD7. Such antibodies may be derived from any species, and may be for example, mouse, rabbit, human, humanized, or camelid antibodies. Exemplary antibodies include grsinilimab, SPV-T3a and those disclosed in WO2015/184941; U.S. Pat. No. 10,106,609; WO2017/213979; WO2018/098306; WO2019086534; U.S. Pat. No. 11,447,548; WO2019/102234; WO2022/136887; WO2022/136888; WO2020/212710; WO2021/160267; WO2022/095803; WO2022/151851. Further exemplary anti-CD7 binding agents and G proteins are described in U.S. provisional application No. 63/172,518, which is incorporated by reference herein. Other exemplary binding agents include designed ankyrin repeat proteins (DARPins) and binding agents based on fibronectin type III (Fn3) scaffolds.

In some embodiments, protein fusogens or attachment proteins may be re-targeted by mutating amino acid residues in a fusion protein or a targeting protein (e.g. retargeted attachment protein). In particular embodiments, the fusogen (e.g. G protein) is mutated to reduce binding for the native binding partner of the fusogen. In some embodiments, the fusogen is or contains a mutant G protein or a biologically active portion thereof that is a mutant of wild-type NiV-G and exhibits reduced binding to one or both of the native binding partners Ephrin B2 or Ephrin B3, including any as described above. Thus, in some aspects, a fusogen can be retargeted to display altered tropism. In some embodiments, the binding confers re-targeted binding compared to the binding of a wild-type surface glycoprotein protein in which a new or different binding activity is conferred. In particular embodiments, the binding confers re-targeted binding compared to the binding of a wild-type G protein in which a new or different binding activity is conferred. In some embodiments the fusogen is randomly mutated. In some embodiments the fusogen is rationally mutated. In some embodiments the fusogen is subjected to directed evolution. In some embodiments the fusogen is truncated and only a subset of the peptide is used in the viral vector. In some embodiments, amino acid residues in the measles hemagglutinin protein may be mutated to alter the binding properties of the protein, redirecting fusion (doi: 10.1038/nbt942, Molecular Therapy vol. 16 no. 8, 1427-1436 August 2008, doi: 10.1038/nbt1060, DOI: 10.1128/JVI.76.7.3558-3563.2002, DOI: 10.1128/JVI.75.17.8016-8020.2001, doi: 10.1073pnas.0604993103).

In some embodiments, protein fusogens (e.g., attachment proteins) may be re-targeted by covalently conjugating a CD7 binding agent to the fusion protein or attachment protein (e.g. retargeted attachment protein). In some embodiments, the fusogen and CD7 binding agent are covalently conjugated by expression of a chimeric protein comprising the fusogen linked to the CD8 binding agent. In some embodiments, a single-chain variable fragment (scFv) can be conjugated to fusogens to redirect fusion activity towards cells that display the scFv binding target (doi: 10.1038/nbt1060, DOI 10.1182/blood-2012-11-468579, doi: 10.1038/nmeth.1514, doi: 10.1006/mthe.2002.0550, HUMAN GENE THERAPY 11:817-826, doi: 10.1038/nbt942, doi: 10.1371/journal.pone.0026381, DOI 10.1186/s12896-015-0142-z). In some embodiments, designed ankyrin repeat proteins (DARPin) can be conjugated to fusogens to redirect fusion activity towards cells that display the DARPin binding target (doi: 10.1038/mt.2013.16, doi: 10.1038/mt.2010.298, doi: 10.4049/jimmunol.1500956), as well as combinations of different DARPins (doi: 10.1038/mto.2016.3). In some embodiments, receptor ligands and antigens can be conjugated to fusogens to redirect fusion activity towards cells that display the target receptor (DOI: 10.1089/hgtb.2012.054, DOI: 10.1128/JVI.76.7.3558-3563.2002). In some embodiments, a targeting protein can also include an antibody or an antigen-binding fragment thereof (e.g., Fab, Fab′, F (ab′) 2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). In some embodiments, protein fusogens may be re-targeted by non-covalently conjugating a CD7 binding agent to the fusion protein or targeting protein (e.g. the hemagglutinin protein). In some embodiments, the fusion protein can be engineered to bind the Fc region of an antibody that targets an antigen on a target cell, redirecting the fusion activity towards cells that display the antibody's target (DOI: 10.1128/JVI.75.17.8016-8020.2001, doi: 10.1038/nm1192). In some embodiments, altered and non-altered fusogens may be displayed on the same retroviral vector or VLP (doi: 10.1016/j.biomaterials.2014.01.051).

In some embodiments, a CD7 binding agent comprises a humanized antibody molecule, intact IgA, IgG, IgE or IgM antibody; bi- or multi-specific antibody (e.g., Zybodies®, etc.); antibody fragments such as Fab fragments, Fab′ fragments, F(ab′) 2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPsTM”); single chain or Tandem diabodies (TandAb®); VHHs; Anticalins®; Nanobodies®; minibodies; BiTE®s; ankyrin repeat proteins or DARPINS®; Avimers®; DARTs; TCR-like antibodies; Adnectins®; Affilins®; Trans-Bodies®; Affibodies®; TrimerX®; MicroProteins; Fynomers®, Centyrins®; and KALBITOR®s.

In some embodiments, the CD7 binding agent is a peptide. In some embodiments, the CD7 binding agent is an antibody, such as a single-chain variable fragment (scFv). In some embodiments, the CD7 binding agent is an antibody, such as a single domain antibody. In some embodiments, the CD7 binding agent is a VHH. In some embodiments, the antibody can be human or humanized. In some embodiments, the antibody or portion thereof is naturally occurring. In some embodiments, the antibody or portion thereof is synthetic.

In some embodiments, the antibody can be generated from phage display libraries to have specificity for a desired target ligand. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, a library of human single domain antibodies is synthetically generated by introducing diversity into one or more scaffolds.

In some embodiments, the C-terminus of the CD7 binding agent is attached to the C-terminus of the G protein (e.g., fusogen) or biologically active portion thereof. In some embodiments, the N-terminus of the CD7 binding agent is exposed on the exterior surface of the lipid bilayer.

In some embodiments, the CD7 binding agent is the only surface displayed non-viral sequence of the viral vector. In some embodiments, the CD7 binding agent is the only membrane bound non-viral sequence of the viral vector. In some embodiments, the viral vector does not contain a molecule that engages or stimulates T cells other than the CD7 binding agent.

In some embodiments, viral vectors may display CD7 binding agents that are not conjugated to protein fusogens in order to redirect the fusion activity towards a cell that is bound by the targeting moiety, or to affect homing.

### iv. CD8 Binding Agents

In some embodiments, the lipid particles disclosed herein comprise one or more retargeted attachment proteins, each independently comprising (i) a paramyxovirus envelope attachment protein; and (ii) a targeting moiety directed to a target molecule expressed on the surface of a target cell, wherein the target molecule is CD8. In some embodiments, the targeting moiety is a CD8 binding domain, e.g., a CD8 binding agent, such as any of those disclosed herein.

The lipid particles disclosed herein include, in some embodiments, one or more CD8 binding agents. For example, a CD8 binding agent may be fused to or incorporated in a protein fusogen or attachment protein. In another embodiment, a CD8 binding agent may be incorporated into the lipid particle envelope via fusion with a transmembrane domain.

Exemplary CD8 binding agents include antibodies and fragments thereof (e.g., scFv, VHH) that bind to one or more of CD8 alpha and CD8 beta. Such antibodies may be derived from any species, and may be for example, mouse, rabbit, human, humanized, or camelid antibodies. Exemplary antibodies include those disclosed in WO2014025828, WO2014164553, WO2020069433, WO2015184203, US20160176969, WO2017134306, WO2019032661, WO2020257412, WO2018170096, WO2020060924, U.S. Pat. No. 10,730,944, US20200172620, and the non-human antibodies OKT8; RPA-T8, 12.C7 (Novus); 17D8, 3B5, LT8, RIV11, SP16, YTC182.20, MEM-31, MEM-87, RAVB3, C8/144B (Thermo Fisher); 2ST8.5H7, Bu88, 3C39, Hit8a, SPM548, CA-8, SK1, RPA-T8 (GeneTex); UCHT4 (Absolute Antibody); BW135/80 (Miltenyi); G42-8 (BD Biosciences); C8/1779R, mAB 104 (Enzo Life Sciences); B-Z31 (Sapphire North America); 32-M4, 5F₁₀, MCD8, UCH-T4, 5F₂ (Santa Cruz); D8A8Y, RPA-T8 (Cell Signaling Technology). Further exemplary anti-CD8 binding agents and G proteins are described in U.S. provisional application No. 63/172,518, which is incorporated by reference herein. Other exemplary binding agents include designed ankyrin repeat proteins (DARPins) and binding agents based on fibronectin type III (Fn3) scaffolds.

In some embodiments, the CD8 binding agent comprises a CDR-H1, a CDR-H2, and a CDR-H3 comprising the amino acid sequence set forth in SEQ ID NO: 483, 484, and 485, respectively; and a CDR-L1, a CDR-L2, and a CDR-L3 comprising the amino acid sequence set forth in SEQ ID NO: 486, 487, and 488, respectively. In some embodiments, the CD8 binding agent comprises a heavy chain variable region (VH) comprising the amino acid sequence set forth in SEQ ID NO:369, and a light chain variable region (VL) comprising the amino acid sequence set forth in SEQ ID NO:370. In some embodiments, the CD8 binding agent comprises the sequence set forth in SEQ ID NO:489.

In some embodiments, the CD8 binding agent comprises a CDR-H1, a CDR-H2, and a CDR-H3 comprising the amino acid sequence set forth in SEQ ID NO: 490, 491, and 492, respectively; and a CDR-L1, a CDR-L2, and a CDR-L3 comprising the amino acid sequence set forth in SEQ ID NO: 493, 494, and 495, respectively. In some embodiments, the CD8 binding agent comprises a heavy chain variable region (VH) comprising the amino acid sequence set forth in SEQ ID NO:371 and a light chain variable region (VL) comprising the amino acid sequence set forth in SEQ ID NO:372. In some embodiments, the CD8 binding agent comprises the sequence set forth in SEQ ID NO:496.

In some embodiments, the CD8 binding agent comprises a CDR-H1, a CDR-H2, and a CDR-H3 comprising the amino acid sequence set forth in SEQ ID NO: 497, 498, and 499, respectively; and a CDR-L1, a CDR-L2, and a CDR-L3 comprising the amino acid sequence set forth in SEQ ID NO: 486, 487, and 500, respectively. In some embodiments, the CD8 binding agent comprises a heavy chain variable region (VH) comprising the amino acid sequence set forth in SEQ ID NO:373, and a light chain variable region (VL) comprising the amino acid sequence set forth in SEQ ID NO:374. In some embodiments, the CD8 binding agent comprises the sequence set forth in SEQ ID NO:501.

In some embodiments, the CD8 binding agent comprises a CDR-H1, a CDR-H2, and a CDR-H3 comprising the amino acid sequence set forth in SEQ ID NO: 502, 503, and 504, respectively; and a CDR-L1, a CDR-L2, and a CDR-L3 comprising the amino acid sequence set forth in SEQ ID NO: 505, 506, and 507, respectively. In some embodiments, the CD8 binding agent comprises a heavy chain variable region (VH) comprising the amino acid sequence set forth in SEQ ID NO:375, and a light chain variable region (VL) comprising the amino acid sequence set forth in SEQ ID NO:376. In some embodiments, the CD8 binding agent comprises the sequence set forth in SEQ ID NO:508.

In some embodiments, the CD8 binding agent comprises a CDR-H1, a CDR-H2, and a CDR-H3 comprising the amino acid sequence set forth in SEQ ID NO: 509, 510, and 511, respectively. In some embodiments, the CD8 binding agent comprises a heavy chain variable region (VH) comprising the amino acid sequence set forth in SEQ ID NO:377. In some embodiments, the CD8 binding agent comprises the sequence set forth in SEQ ID NO:377.

In some embodiments, the CD8 binding agent comprises any CD8 binding agent as described in US 2019/0144885, incorporated by reference herein in its entirety.

In some embodiments, the CD8 binding agent is an scFv that contains a VH and VL set forth from any as below, in which the VH and VL are separated by linker. In some embodiments, the CD8 binding agent is a VHH having the sequence set forth below. In some embodiments, the CD8 binding agent is linked to the C-terminus of a truncated NiV-G set forth in SEQ ID NO: 19 to provide a re-targeted NiV-G. In some embodiments, the retargeted NiV-G is pseudotyped on a lentiviral vector with the a NiV-F (e.g. set forth in SEQ ID NO:227). In some embodiments, the lentiviral vector further contains a payload gene encoding an anti-CD19 CAR. In some embodiments, the anti-CD19 CAR contains an anti-CD19 FMC63 scFv binding domain set forth in SEQ ID NO:239, a CD8 hinge set forth in SEQ ID NO:367, a CD8 transmembrane domain set forth in SEQ ID NO: 368, a 4-1bb signaling domain set forth in SEQ ID NO:248. a CD3zeta signaling domain set forth in SEQ ID NO: 249.

CD8_1
VH (SEQ ID NO.: 369):
QVQLVQSGAEVKKPGASVKVSCKASGGTFSSYAISWVRQAPGQGLEW
MGIIDPSDGNTNYAQNFQGRVTMTRDTSTSTVYMELSSLRSEDTAVY
YCAKERAAAGYYYYMDVWGQGTTVTVSS
VL (SEQ ID NO.: 370):
DIQMTQSPSSLSASVGDRVTITCRASQSISSYLNWYQQKPGKAPKLL
IYAASSLQSGVPSRFSGSGSGTDFTLTISSLQPEDFATYYCQQSYST
PLTFGGGTKVEIKR
CD8_2
VH (SEQ ID NO.: 371):
QVQLVQSGAEVKKPGASVKVSCKASGYTFTDYYIQWVRQAPGQGLEW
MGWINPNSGGTSYAQKFQGRVTMTRDTSTSTVYMELSSLRSEDTAVY
YCAKEGDYYYGMDAWGQGTMVTVSS
VL (SEQ ID NO.: 372):
DIVMTQSPLSLPVTPGEPASISCRSSQSLLHSNGYNYLDWYLQKPGQ
SPQLLIYLGSNRASGVPDRFSGSGSGTDFTLKISRVEAEDVGVYYCM
QGLQTPHTFGQGTKVEIKR
CD8_3
VH (SEQ ID NO.: 373):
QVQLVQSGAEVKKPGASVKVSCKASGYTFTSYYMHWVRQAPGQGLEW
MGGFDPEDGETIYAQKFQGRVTMTRDTSTSTVYMELSSLRSEDTAVY
YCARDQGWGMDVWGQGTTVTVSS
VL(SEQ ID NO.: 374):
DIQMTQSPSSLSASVGDRVTITCRASQSISSYLNWYQQKPGKAPKLL
IYAASSLQSGVPSRFSGSGSGTDFTLTISSLQPEDFATYYCQQTYST
PYTFGQGTKLEIKR
CD8_4
VH (SEQ ID NO.: 375):
QVQLVQSGAEVKKPGASVKVSCKASGYTFTNHYMHWVRQAPGQGLEW
MGWMNPNSGNTGYAQKFQGRVTMTRDTSTSTVYMELSSLRSEDTAVY
YCASSESGSDLDYWGQGTLVTVSS
VL (SEQ ID NO.: 376):
DIQMTQSPSSLSASVGDRVTITCRASQTIGNYVNWYQQKPGKAPKLL
IYGASNLHTGVPSRFSGSGSGTDFTLTISSLQPEDFATYYCQQTYSA
PLTFGGGTKVEIKR
In some embodiments, the CD8 binding agent is
VHH set forth as:
VHH (SEQ ID NO.: 377):
QVQLVESGGGLVQAGGSLRLSCAASGRTFSGYVMGWFRQAPGKQRKF
VAAISRGGLSTSYADSVKGRFTISRDNAKNTVFLQMNTLKPEDTAVY
YCAADRSDLYEITAASNIDSWGQGTLVTVSS

In some embodiments, protein fusogens or attachment proteins may be re-targeted by mutating amino acid residues in a fusion protein or a targeting protein (e.g. retargeted attachment protein). In particular embodiments, the fusogen (e.g. G protein) is mutated to reduce binding for the native binding partner of the fusogen. In some embodiments, the fusogen is or contains a mutant G protein or a biologically active portion thereof that is a mutant of wild-type NiV-G and exhibits reduced binding to one or both of the native binding partners Ephrin B2 or Ephrin B3, including any as described above. Thus, in some aspects, a fusogen can be retargeted to display altered tropism. In some embodiments, the binding confers re-targeted binding compared to the binding of a wild-type surface glycoprotein protein in which a new or different binding activity is conferred. In particular embodiments, the binding confers re-targeted binding compared to the binding of a wild-type G protein in which a new or different binding activity is conferred. In some embodiments the fusogen is randomly mutated. In some embodiments the fusogen is rationally mutated. In some embodiments the fusogen is subjected to directed evolution. In some embodiments the fusogen is truncated and only a subset of the peptide is used in the viral vector. In some embodiments, amino acid residues in the measles hemagglutinin protein may be mutated to alter the binding properties of the protein, redirecting fusion (doi: 10.1038/nbt942, Molecular Therapy vol. 16 no. 8, 1427-1436 August 2008, doi: 10.1038/nbt1060, DOI: 10.1128/JVI.76.7.3558-3563.2002, DOI: 10.1128/JVI.75.17.8016-8020.2001, doi: 10.1073pnas.0604993103).

In some embodiments, protein fusogens (e.g., attachment proteins) may be re-targeted by covalently conjugating a CD8 binding agent to the fusion protein or attachment protein (e.g. retargeted attachment protein). In some embodiments, the fusogen and CD8 binding agent are covalently conjugated by expression of a chimeric protein comprising the fusogen linked to the CD8 binding agent. In some embodiments, a single-chain variable fragment (scFv) can be conjugated to fusogens to redirect fusion activity towards cells that display the scFv binding target (doi: 10.1038/nbt1060, DOI 10.1182/blood-2012-11-468579, doi: 10.1038/nmeth.1514, doi: 10.1006/mthe.2002.0550, HUMAN GENE THERAPY 11:817-826, doi: 10.1038/nbt942, doi: 10.1371/journal.pone.0026381, DOI 10.1186/s12896-015-0142-z). In some embodiments, designed ankyrin repeat proteins (DARPin) can be conjugated to fusogens to redirect fusion activity towards cells that display the DARPin binding target (doi: 10.1038/mt.2013.16, doi: 10.1038/mt.2010.298, doi: 10.4049/jimmunol.1500956), as well as combinations of different DARPins (doi: 10.1038/mto.2016.3). In some embodiments, receptor ligands and antigens can be conjugated to fusogens to redirect fusion activity towards cells that display the target receptor (DOI: 10.1089/hgtb.2012.054, DOI: 10.1128/JVI.76.7.3558-3563.2002). In some embodiments, a targeting protein can also include an antibody or an antigen-binding fragment thereof (e.g., Fab, Fab′, F (ab′) 2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). In some embodiments, protein fusogens may be re-targeted by non-covalently conjugating a CD8 binding agent to the fusion protein or targeting protein (e.g. the hemagglutinin protein). In some embodiments, the fusion protein can be engineered to bind the Fc region of an antibody that targets an antigen on a target cell, redirecting the fusion activity towards cells that display the antibody's target (DOI: 10.1128/JVI.75.17.8016-8020.2001, doi: 10.1038/nm1192). In some embodiments, altered and non-altered fusogens may be displayed on the same retroviral vector or VLP (doi: 10.1016/j.biomaterials.2014.01.051).

In some embodiments, a CD8 binding agent comprises a humanized antibody molecule, intact IgA, IgG, IgE or IgM antibody; bi- or multi-specific antibody (e.g., Zybodies®, etc.); antibody fragments such as Fab fragments, Fab′ fragments, F(ab′) 2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPsTM”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-Bodies®; Affibodies®; TrimerX®; MicroProteins; Fynomers®, Centyrins®; and KALBITOR®s.

In some embodiments, the CD8 binding agent is a peptide. In some embodiments, the CD8 binding agent is an antibody, such as a single-chain variable fragment (scFv). In some embodiments, the CD8 binding agent is an antibody, such as a single domain antibody. In some embodiments, the CD8 binding agent is a VHH. In some embodiments, the antibody can be human or humanized. In some embodiments, the antibody or portion thereof is naturally occurring. In some embodiments, the antibody or portion thereof is synthetic.

In some embodiments, the antibody can be generated from phage display libraries to have specificity for a desired target ligand. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, a library of human single domain antibodies is synthetically generated by introducing diversity into one or more scaffolds.

In some embodiments, the C-terminus of the CD8 binding agent is attached to the C-terminus of the G protein (e.g., fusogen) or biologically active portion thereof. In some embodiments, the N-terminus of the CD8 binding agent is exposed on the exterior surface of the lipid bilayer.

In some embodiments, the CD8 binding agent is the only surface displayed non-viral sequence of the viral vector. In some embodiments, the CD8 binding agent is the only membrane bound non-viral sequence of the viral vector. In some embodiments, the viral vector does not contain a molecule that engages or stimulates T cells other than the CD8 binding agent.

In some embodiments, viral vectors may display CD8 binding agents that are not conjugated to protein fusogens in order to redirect the fusion activity towards a cell that is bound by the targeting moiety, or to affect homing.

Non-limiting examples of re-targeted fusogens comprising a CD8 binding agent are as described in U.S. Pat. No. 11,535,869, the entire contents of which is incorporated by reference herein.

### b. HSC Binding Domains

In some embodiments, the lipid particles disclosed herein comprise one or more retargeted attachment proteins, each independently comprising (i) a paramyxovirus envelope attachment protein; and (ii) a targeting moiety directed to a target molecule expressed on the surface of a target cell. In some embodiments, the targeting moiety is an HSC binding domain, e.g., an HSC binding agent, such as any of those disclosed herein.

The lipid particles disclosed herein include, in some embodiments, one or more HSC binding domains (e.g., HSC binding agent) that target the viral vector to a cell that is an HSC. In some embodiments, the HSC binding agent binds to a molecule expressed on the surface of the HSC. The cell surface molecule may be a receptor, coreceptor, or a GPI-anchored protein. In some embodiments, the HSC binding agent binds ASCT2, CD105, CD110, CD117, CD133, CD146, CD164, CD34, CD46, CD49f, CD90, EPCR, or ITGA3. In some embodiments, a HSC binding agent may be fused to or incorporated in a protein fusogen or lipid particle envelope attachment protein (e.g., a retargeted attachment protein). In some embodiments, a HSC binding agent may be incorporated into the viral envelope via fusion with a transmembrane domain. In some embodiments, the HSC binding agent targets the lipid particle to a HSC.

In particular embodiments, a HSC binding agent may be fused to or incorporated in a protein fusogen or attachment protein, thereby retargeting the lipid particle to a HSC. In some embodiments, for re-targeting the HSC binding agent is fused to a protein fusogen or envelope attachment protein that is mutated to reduce binding for the native binding partner of the fusogen or viral envelope protein. In some embodiments, the fusogen is or contains a mutant G protein or a biologically active portion thereof that is a mutant of wild-type NiV-G and exhibits reduced binding to one or both of the native binding partners Ephrin B2 or Ephrin B3, including any as described above. Thus, in some aspects, a fusogen can be retargeted to display altered tropism. In some embodiments, the binding confers re-targeted binding compared to the binding of a wild-type surface glycoprotein protein in which a new or different binding activity is conferred. In particular embodiments, the binding confers re-targeted binding compared to the binding of a wild-type G protein in which a new or different binding activity is conferred. In some embodiments the fusogen is randomly mutated. In some embodiments the fusogen is rationally mutated. In some embodiments the fusogen is subjected to directed evolution. In some embodiments the fusogen is truncated and only a subset of the peptide is used in the viral vector. In some embodiments, amino acid residues in the measles hemagglutinin protein may be mutated to alter the binding properties of the protein, redirecting fusion (doi: 10.1038/nbt942, Molecular Therapy vol. 16 no. 8, 1427-1436 August 2008, doi: 10.1038/nbt1060, DOI: 10.1128/JVI.76.7.3558-3563.2002, DOI: 10.1128/JVI.75.17.8016-8020.2001, doi: 10.1073pnas.0604993103).

In some embodiments, protein fusogens may be re-targeted by covalently conjugating a HSC binding agent to the attachment protein. In some embodiments, the fusogen and HSC binding agent are covalently conjugated by expression of a chimeric protein comprising the fusogen linked to the HSC binding agent (e.g., retargeted attachment protein). The HSC binding agent can include any targeting protein able to confer specific binding to a target molecule expressed on the surface of a HSC. In some embodiments, a targeting protein can also include an antibody or an antigen-binding fragment thereof (e.g., Fab, Fab′, F(ab′) 2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). In some embodiments, the HSC binding agent is an antibody or antigen binding fragment thereof. In some embodiments, the fusion protein can be engineered to bind the Fc region of an antibody that targets an antigen on a target cell, redirecting the fusion activity towards cells that display the antibody's target (DOI: 10.1128/JVI.75.17.8016-8020.2001, doi: 10.1038/nm1192). In some embodiments, altered and non-altered fusogens may be displayed on the same retroviral vector or VLP (doi: 10.1016/j.biomaterials.2014.01.051).

In some embodiments, a single-chain variable fragment (scFv) can be conjugated to fusogens to redirect fusion activity towards HSCs that display the scFv binding target (doi: 10.1038/nbt1060, DOI 10.1182/blood-2012-11-468579, doi: 10.1038/nmeth.1514, doi: 10.1006/mthe.2002.0550, HUMAN GENE THERAPY 11:817-826, doi: 10.1038/nbt942, doi: 10.1371/journal.pone.0026381, DOI 10.1186/s12896-015-0142-z). In some embodiments, designed ankyrin repeat proteins (DARPin) can be conjugated to fusogens to redirect fusion activity towards HSCs that display the DARPin binding target (doi: 10.1038/mt.2013.16, doi: 10.1038/mt.2010.298, doi: 10.4049/jimmunol.1500956), as well as combinations of different DARPins (doi: 10.1038/mto.2016.3). In some embodiments, a single domain antibody (e.g., a VHH) can be conjugated to fusogens to redirect fusion activity towards HSCs that display the sdAb binding target. In some embodiments, receptor ligands and antigens can be conjugated to fusogens to redirect fusion activity towards HSCs that display the target receptor (DOI: 10.1089/hgtb.2012.054, DOI: 10.1128/JVI.76.7.3558-3563.2002).

In some embodiments, the target cell is a CD34+ progenitor cells. In some embodiments, the target cell molecule is expressed on at least a subset of CD34+ progenitor cells.

In some embodiments, the cell surface molecule is expressed on HSCs. In some embodiments, the cell surface molecule is expressed on MPPs. In some embodiments, the cell surface molecule is expressed on MLPs. In some embodiments, the cell surface molecule is expressed on ETPs. In some embodiments, the cell surface molecule is expressed on MEPs. In some embodiments, the cell surface molecule is expressed on CMPs. In some embodiments, the cell surface molecule is expressed on GMPs. In some embodiments, the cell surface molecule is expressed on any combination of the foregoing CD34+ progenitor subpopulations. In some embodiments, the cell surface molecule is expressed on HSCs and MPPs. In some embodiments, the cell surface molecule is expressed on myeloid progenitors. In some embodiments, the cell surface molecule is expressed on lymphoid progenitors. In some embodiments, the cell surface molecule is expressed on myeloid progenitors. In some embodiments, the cell surface molecule is expressed on HSCs, MPPs, MEPs, CMPs, and GMPs.

In some embodiments, the cell surface molecule is ASCT2. In some embodiments, the target cell is ASCT2+.

In some embodiments, the cell surface molecule is CD105. In some embodiments, the target cell is CD105+.

In some embodiments, the cell surface molecule is CD110. In some embodiments, the target cell is CD110+.

In some embodiments, the cell surface molecule is CD117. In some embodiments, the target cell is CD117+.

In some embodiments, the cell surface molecule is CD133. In some embodiments, the target cell is CD133+.

In some embodiments, the cell surface molecule is CD146. In some embodiments, the target cell is CD146+.

In some embodiments, the cell surface molecule is CD164. In some embodiments, the target cell is CD164+.

In some embodiments, the cell surface molecule is CD34. In some embodiments, the target cell is CD34+.

In some embodiments, the cell surface molecule is CD46. In some embodiments, the target cell is CD46+.

In some embodiments, the cell surface molecule is CD49f. In some embodiments, the target cell is CD49f+.

In some embodiments, the ta cell surface molecule is CD90. In some embodiments, the target cell is CD90+.

In some embodiments, the cell surface molecule is EPCR. In some embodiments, the target cell is EPCR+.

In some embodiments, the cell surface molecule is ITGA3. In some embodiments, the target cell is ITGA3+.

In some embodiments, the target molecule is CD133. In some embodiments, the target cell is CD133+. In some embodiments, the targeting agent is an anti-CD133 antibody. Exemplary anti-CD133 antibodies include CART133, AC133, 293C3-SDIE, CMab-43, RW03, 293C3H9 (293C3), and W6B3H10 (W6B3); and anti-CD133 antibodies disclosed in U.S. Pat. Nos. 8,722,858, 9,249,225, 9,624,303, 10,106,623, 10,711,068, 11,098,109, 11,214,628, 11,352,435, and 11,220,551; US Patent Application Nos. US20130224202; PCT Application Nos. WO200901840, WO2011089211, WO2011149493, WO2014128185, WO2015121383, WO2016154623, WO2018045880, WO2018072025, and WO2022022718; and Canadian Patent Application No. CA2962157.

In some embodiments, the lipid particles disclosed herein comprise one or more retargeted attachment proteins, each independently comprising (i) a paramyxovirus envelope attachment protein; and (ii) a targeting moiety directed to a target molecule expressed on the surface of a target cell, wherein the target molecule is CD133. In some embodiments, the targeting moiety is a CD133 binding domain, e.g., a CD133 binding agent, such as any of those disclosed herein.

In some embodiments, the lipid particles comprise one or more HSC binding domains that is a CD133 binding agent that targets the viral vector to a cell that is an HSC. In some embodiments, the lipid particles comprise two or more HSC binding domains that are each a CD133 binding agent that targets the viral vector to a cell that is an HSC. In some embodiments, each of the two or more HSC binding domains that are each a CD133 binding agent bind distinct epitopes of the same target molecule (CD133). In some embodiments, the lipid particle comprises two or more, e.g., two, three, four, or five or more, CD133 binding agents.

In some embodiments, the lipid particle comprises one or more targeting moieties, e.g., HSC binding domains, selected from: (a) a CD133 binding agent comprising a CDR-H1, a CDR-H2, and a CDR-H3 comprising the amino acid sequences of SEQ ID NOs: 536, 537, and 538, respectively, and a CDR-L1, a CDR-L2, and a CDR-L3 comprising the amino acid sequences of SEQ ID NOs: 540, 541, and 542, respectively; (b) a CD133 binding agent comprising a CDR-H1, a CDR-H2, and a CDR-H3 comprising the amino acid sequences of SEQ ID NOs: 545, 546, and 547, respectively, and a CDR-L1, a CDR-L2, and a CDR-L3 comprising the amino acid sequences of SEQ ID NOs: 549, 550, and 551, respectively; (c) a CD133 binding agent comprising a CDR-H1, a CDR-H2, and a CDR-H3 comprising the amino acid sequences of SEQ ID NOs: 518, 519, and 520, respectively, and a CDR-L1, a CDR-L2, and a CDR-L3 comprising the amino acid sequences of SEQ ID NOs: 522, 523, and 524, respectively; (d) and a CD133 binding agent comprising a CDR-H1, a CDR-H2, and a CDR-H3 comprising the amino acid sequences of SEQ ID NOs: 527, 528, and 529, respectively, and a CDR-L1, a CDR-L2, and a CDR-L3 comprising the amino acid sequences of SEQ ID NOs: 531, 532, and 533, respectively; and (e) and a CD133 binding agent comprising a CDR-H1, a CDR-H2, and a CDR-H3 comprising the amino acid sequences of SEQ ID NOs: 554, 555, and 556, respectively, and a CDR-L1, a CDR-L2, and a CDR-L3 comprising the amino acid sequences of SEQ ID NOs: 558, 559, and 560, respectively.

In some embodiments, the lipid particle comprises one or more targeting moieties, e.g., HSC binding domains, selected from: (a) a CD133 binding agent comprising a CDR-H1, a CDR-H2, and a CDR-H3 comprising the amino acid sequences of SEQ ID NOs: 289, 565, and 538, respectively, and a CDR-L1, a CDR-L2, and a CDR-L3 comprising the amino acid sequences of SEQ ID NOs: 540, 541, and 542, respectively; (b) a CD133 binding agent comprising a CDR-H1, a CDR-H2, and a CDR-H3 comprising the amino acid sequences of SEQ ID NOs: 566, 567, and 547, respectively, and a CDR-L1, a CDR-L2, and a CDR-L3 comprising the amino acid sequences of SEQ ID NOs: 549, 550, and 551, respectively; (c) a CD133 binding agent comprising a CDR-H1, a CDR-H2, and a CDR-H3 comprising the amino acid sequences of SEQ ID NOs: 568, 569, and 520, respectively, and a CDR-L1, a CDR-L2, and a CDR-L3 comprising the amino acid sequences of SEQ ID NOs: 522, 523, and 524, respectively; (d) and a CD133 binding agent comprising a CDR-H1, a CDR-H2, and a CDR-H3 comprising the amino acid sequences of SEQ ID NOs: 570, 571, and 529, respectively, and a CDR-L1, a CDR-L2, and a CDR-L3 comprising the amino acid sequences of SEQ ID NOs: 531, 532, and 533, respectively; and (e) and a CD133 binding agent comprising a CDR-H1, a CDR-H2, and a CDR-H3 comprising the amino acid sequences of SEQ ID NOs: 572, 573, and 556, respectively, and a CDR-L1, a CDR-L2, and a CDR-L3 comprising the amino acid sequences of SEQ ID NOs: 558, 559, and 560, respectively.

In some embodiments, the lipid particle comprises one or more, e.g., one, two, three, or more, targeting moieties, e.g., HSC binding domains, independently selected from: (a) a CD133 binding agent comprising a heavy chain variable (VH) region comprising the amino acid sequence of SEQ ID NO: 535, or an amino acid sequence having at least 90%, 91%, 92%, 93%, 94%, 95%, 96%, 97%, 98%, or 99% sequence identity thereto; and a light chain variable (VL) region comprising the amino acid sequence of SEQ ID NO: 539, or an amino acid sequence having at least 90%, 91%, 92%, 93%, 94%, 95%, 96%, 97%, 98%, or 99% sequence identity thereto; (b) a CD133 binding agent comprising a VH region comprising the amino acid sequence of SEQ ID NO: 544, or an amino acid sequence having at least 90%, 91%, 92%, 93%, 94%, 95%, 96%, 97%, 98%, or 99% sequence identity thereto; and a VL region comprising the amino acid sequence of SEQ ID NO: 548, or an amino acid sequence having at least 90%, 91%, 92%, 93%, 94%, 95%, 96%, 97%, 98%, or 99% sequence identity thereto; (c) a CD133 binding agent comprising a VH region comprising the amino acid sequence of SEQ ID NO: 517, or an amino acid sequence having at least 90%, 91%, 92%, 93%, 94%, 95%, 96%, 97%, 98%, or 99% sequence identity thereto; and a VL region comprising the amino acid sequence of SEQ ID NO: 521, or an amino acid sequence having at least 90%, 91%, 92%, 93%, 94%, 95%, 96%, 97%, 98%, or 99% sequence identity thereto; (d) a CD133 binding agent comprising a VH region comprising the amino acid sequence of SEQ ID NO: 526, or an amino acid sequence having at least 90%, 91%, 92%, 93%, 94%, 95%, 96%, 97%, 98%, or 99% sequence identity thereto; and a VL region comprising the amino acid sequence of SEQ ID NO: 530, or an amino acid sequence having at least 90%, 91%, 92%, 93%, 94%, 95%, 96%, 97%, 98%, or 99% sequence identity thereto; and (e) a CD133 binding agent comprising a VH region comprising the amino acid sequence of SEQ ID NO: 553, or an amino acid sequence having at least 90%, 91%, 92%, 93%, 94%, 95%, 96%, 97%, 98%, or 99% sequence identity thereto; and a VL region comprising the amino acid sequence of SEQ ID NO: 557, or an amino acid sequence having at least 90%, 91%, 92%, 93%, 94%, 95%, 96%, 97%, 98%, or 99% sequence identity thereto.

In some embodiments, the lipid particle comprises one or more targeting moieties, e.g., HSC binding domains, comprising an amino acid sequence independently selected from the group consisting of SEQ ID NOs: 516, 525, 534, 543, and 552, or an amino acid sequence having at least 90%, 91%, 92%, 93%, 94%, 95%, 96%, 97%, 98%, or 99% sequence identity thereto. In some embodiments, each of the one or more CD133 binding agents is independently an scFv comprising an amino acid sequence selected from the group consisting of SEQ ID NOs: 516, 525, 534, 543, and 552, or an amino acid sequence having at least 90%, 91%, 92%, 93%, 94%, 95%, 96%, 97%, 98%, or 99% sequence identity thereto.

In some embodiments, the lipid particle comprises a first CD133 binding agent that is an scFv comprising the amino acid sequence of SEQ ID NO: 516 or an amino acid sequence having at least 90%, 91%, 92%, 93%, 94%, 95%, 96%, 97%, 98%, or 99% sequence identity thereto, and a second CD133 binding agent that is an scFv comprising the amino acid sequence of SEQ ID NO: 525 or an amino acid sequence having at least 90%, 91%, 92%, 93%, 94%, 95%, 96%, 97%, 98%, or 99% sequence identity thereto.

In some embodiments, the target molecule is CD105. In some embodiments, the target cell is CD105+. In some embodiments, the targeting agent is an anti-CD105 antibody. Exemplary anti-CD105 antibodies include carotuximab, TRC105, huRH105, and TCR205; and anti-CD105 antibodies disclosed in U.S. Pat. Nos. 8,221,753, 8,609,094, 9,150,652, 95,181,212, 9,926,375, 9,944,714, 10,155,820, and 10,336,831; US Patent Application Nos. US20100098692, US20100196398, US20170007714, and US20220233591; PCT Application Nos. WO2010039873, WO2011041441, WO2016077451, WO2018067819, WO2010032059, WO2012149412, WO2015118031, WO2021118955, and WO2021118957; and Korean Patent No. KR101398707B1.

In some embodiments, the lipid particles disclosed herein comprise one or more retargeted attachment proteins, each independently comprising (i) a paramyxovirus envelope attachment protein; and (ii) a targeting moiety directed to a target molecule expressed on the surface of a target cell, wherein the target molecule is CD117. In some embodiments, the targeting moiety is a CD117 binding domain, e.g., a CD117 binding agent, such as any of those disclosed herein.

In some embodiments, the target molecule is CD117. In some embodiments, the target cell is CD117+. In some embodiments, the targeting agent is an anti-CD117 antibody. In some embodiments, the lipid particles comprise one or more HSC binding domains that is a CD117 binding agent that targets the viral vector to a cell that is an HSC. In some embodiments, the lipid particle comprises one or more targeting moieties, e.g., HSC binding domains, that is a CD117 binding agent comprising an amino acid sequence independently selected from the group consisting of SEQ ID NOs: 512-515, or an amino acid sequence having at least 90%, 91%, 92%, 93%, 94%, 95%, 96%, 97%, 98%, or 99% sequence identity thereto. In some embodiments, the CD117 binding agent comprises an amino acid sequence selected from the group consisting of SEQ ID NOs: 593-639, or an amino acid sequence having at least 90%, 91%, 92%, 93%, 94%, 95%, 96%, 97%, 98%, or 99% sequence identity thereto.

In some embodiments, the CD117 binding agent is a VHH comprising a CDR-H1, a CDR-H2, and a CDR-H3 contained within an amino acid sequence selected from the group consisting of SEQ ID NOs: 512-515, 593-639, wherein the target molecule is CD117.

In some embodiments, the CD117 binding agent is a VHH comprising the amino acid sequence of SEQ ID NO: 593, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 594, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 595, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 596, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 597, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 598, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 599, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 600, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 601, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 602, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 603, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 512, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 604, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 605, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 606, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 607, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 608, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 609, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 610, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 611, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 612, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 613, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 614, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 615, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 616, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 617, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 618, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 619, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 620, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 353, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 622, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 623, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 624, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 513, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 625, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 626, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 627, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 628, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 629, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 630, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 514, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 631, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 632, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 633, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 634, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 515, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 635, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 636, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 637, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 638, or an amino acid sequence having at least 90%, 91%, 92%, 93%, 94%, 95%, 96%, 97%, 98%, or 99% sequence identity thereto. In some embodiments, the CD117 binding agent is a VHH comprising the amino acid sequence of SEQ ID NO: 639, or an amino acid sequence having at least 90%, 91%, 92%, 93%, 94%, 95%, 96%, 97%, 98%, or 99% sequence identity thereto.

In some embodiments, the lipid particle comprises one or more targeting moieties, e.g., HSC binding domains, selected from: (a) a CD117 binding agent comprising a VHH comprising the amino acid sequence of SEQ ID NO: 512, or an amino acid sequence having at least 90%, 91%, 92%, 93%, 94%, 95%, 96%, 97%, 98%, or 99% sequence identity thereto; (b) a CD117 binding agent comprising a VHH comprising the amino acid sequence of SEQ ID NO: 513, or an amino acid sequence having at least 90%, 91%, 92%, 93%, 94%, 95%, 96%, 97%, 98%, or 99% sequence identity thereto; (c) a CD117 binding agent comprising a VHH comprising the amino acid sequence of SEQ ID NO: 514, or an amino acid sequence having at least 90%, 91%, 92%, 93%, 94%, 95%, 96%, 97%, 98%, or 99% sequence identity thereto; and (d) a CD117 binding agent comprising a VHH comprising the amino acid sequence of SEQ ID NO: 515, or an amino acid sequence having at least 90%, 91%, 92%, 93%, 94%, 95%, 96%, 97%, 98%, or 99% sequence identity thereto.

In some embodiments, the CD117 binding agent is a VHH comprising the amino acid sequence of SEQ ID NO: 593. In some embodiments, the CD117 binding agent is a VHH comprising the amino acid sequence of SEQ ID NO: 594. In some embodiments, the CD117 binding agent is a VHH comprising the amino acid sequence of SEQ ID NO: 595. In some embodiments, the CD117 binding agent is a VHH comprising the amino acid sequence of SEQ ID NO: 596. In some embodiments, the CD117 binding agent is a VHH comprising the amino acid sequence of SEQ ID NO: 597. In some embodiments, the CD117 binding agent is a VHH comprising the amino acid sequence of SEQ ID NO: 598. In some embodiments, the CD117 binding agent is a VHH comprising the amino acid sequence of SEQ ID NO: 599. In some embodiments, the CD117 binding agent is a VHH comprising the amino acid sequence of SEQ ID NO: 600. In some embodiments, the CD117 binding agent is a VHH comprising the amino acid sequence of SEQ ID NO: 601. In some embodiments, the CD117 binding agent is a VHH comprising the amino acid sequence of SEQ ID NO: 602. In some embodiments, the CD117 binding agent is a VHH comprising the amino acid sequence of SEQ ID NO: 603. In some embodiments, the CD117 binding agent is a VHH comprising the amino acid sequence of SEQ ID NO: 512. In some embodiments, the CD117 binding agent is a VHH comprising the amino acid sequence of SEQ ID NO: 604 In some embodiments, the CD117 binding agent is a VHH comprising the amino acid sequence of SEQ ID NO: 605. In some embodiments, the CD117 binding agent is a VHH comprising the amino acid sequence of SEQ ID NO: 606. In some embodiments, the CD117 binding agent is a VHH comprising the amino acid sequence of SEQ ID NO: 607. In some embodiments, the CD117 binding agent is a VHH comprising the amino acid sequence of SEQ ID NO: 608. In some embodiments, the CD117 binding agent is a VHH comprising the amino acid sequence of SEQ ID NO: 609. In some embodiments, the CD117 binding agent is a VHH comprising the amino acid sequence of SEQ ID NO: 610. In some embodiments, the CD117 binding agent is a VHH comprising the amino acid sequence of SEQ ID NO: 611. In some embodiments, the CD117 binding agent is a VHH comprising the amino acid sequence of SEQ ID NO: 612. In some embodiments, the CD117 binding agent is a VHH comprising the amino acid sequence of SEQ ID NO: 613. In some embodiments, the CD117 binding agent is a VHH comprising the amino acid sequence of SEQ ID NO: 614. In some embodiments, the CD117 binding agent is a VHH comprising the amino acid sequence of SEQ ID NO: 615. In some embodiments, the CD117 binding agent is a VHH comprising the amino acid sequence of SEQ ID NO: 616. In some embodiments, the CD117 binding agent is a VHH comprising the amino acid sequence of SEQ ID NO: 617. In some embodiments, the CD117 binding agent is a VHH comprising the amino acid sequence of SEQ ID NO: 618. In some embodiments, the CD117 binding agent is a VHH comprising the amino acid sequence of SEQ ID NO: 619. In some embodiments, the CD117 binding agent is a VHH comprising the amino acid sequence of SEQ ID NO: 620. In some embodiments, the CD117 binding agent is a VHH comprising the amino acid sequence of SEQ ID NO: 621. In some embodiments, the CD117 binding agent is a VHH comprising the amino acid sequence of SEQ ID NO: 622. In some embodiments, the CD117 binding agent is a VHH comprising the amino acid sequence of SEQ ID NO: 623. In some embodiments, the CD117 binding agent is a VHH comprising the amino acid sequence of SEQ ID NO: 624. In some embodiments, the CD117 binding agent is a VHH comprising the amino acid sequence of SEQ ID NO: 513. In some embodiments, the CD117 binding agent is a VHH comprising the amino acid sequence of SEQ ID NO: 625. In some embodiments, the CD117 binding agent is a VHH comprising the amino acid sequence of SEQ ID NO: 626. In some embodiments, the CD117 binding agent is a VHH comprising the amino acid sequence of SEQ ID NO: 627. In some embodiments, the CD117 binding agent is a VHH comprising the amino acid sequence of SEQ ID NO: 628. In some embodiments, the CD117 binding agent is a VHH comprising the amino acid sequence of SEQ ID NO: 629. In some embodiments, the CD117 binding agent is a VHH comprising the amino acid sequence of SEQ ID NO: 630. In some embodiments, the CD117 binding agent is a VHH comprising the amino acid sequence of SEQ ID NO: 514. In some embodiments, the CD117 binding agent is a VHH comprising the amino acid sequence of SEQ ID NO: 631. In some embodiments, the CD117 binding agent is a VHH comprising the amino acid sequence of SEQ ID NO: 632. In some embodiments, the CD117 binding agent is a VHH comprising the amino acid sequence of SEQ ID NO: 633. In some embodiments, the CD117 binding agent is a VHH comprising the amino acid sequence of SEQ ID NO: 634. In some embodiments, the CD117 binding agent is a VHH comprising the amino acid sequence of SEQ ID NO: 515. In some embodiments, the CD117 binding agent is a VHH comprising the amino acid sequence of SEQ ID NO: 635. In some embodiments, the CD117 binding agent is a VHH comprising the amino acid sequence of SEQ ID NO: 636. In some embodiments, the CD117 binding agent is a VHH comprising the amino acid sequence of SEQ ID NO: 637. In some embodiments, the CD117 binding agent is a VHH comprising the amino acid sequence of SEQ ID NO: 638. In some embodiments, the CD117 binding agent is a VHH comprising the amino acid sequence of SEQ ID NO: 639.

In some embodiments, the target molecule is EPCR. In some embodiments, the target cell is EPCR+. In some embodiments, the targeting agent is an anti-EPCR antibody. Exemplary anti-EPCR antibodies include JRK1494, JRK1535; and anti-EPCR antibodies disclosed in US Patent Application Nos. US20210355231 and US20220127374; and PCT Application Nos. WO2020051277 and WO2020161478.

In some embodiments, the target molecule is CD34. In some embodiments, the target cell is CD34+. In some embodiments, the targeting agent is an anti-CD34 antibody. Exemplary anti-CD34 antibodies include h4C8, 9C5; and anti-CD34 antibodies disclosed in U.S. Pat. Nos. 8,399,249, 8,927,696, and 10,106,623; US Patent Application Nos. US20090221003, US20130143238, US20100311955, US20130172533, US20170320966, US20170298148, US20180169177, US20190135945; and PCT Application Nos. WO2009079922 and WO2015121383.

In some embodiments, the target molecule is ASCT2. In some embodiments, the target cell is ASCT2+. In some embodiments, the targeting agent is an anti-ASCT2 antibody. Exemplary anti-ASCT2 antibodies include idactamab, MEDI7247, KM4008, KM4012, KM4018; and anti-ASCT2 antibodies disclosed in U.S. Pat. Nos. 8,268,592, 8,501,180, 8,945,870, 8,673,592, and 10,829,554; US Patent Application Nos. US20180273617, US20190367605, US20210024629; and PCT Application Nos. WO2017083451, WO2018089393.

In some embodiments, the target molecule is CD90. In some embodiments, the target cell is CD90+. In some embodiments, the targeting agent is an anti-CD90 antibody. Exemplary anti-CD90 antibodies include EPR3133, CL1028, CL1040, AF-9, JF10-09, 5E10, 7E1B11; and anti-CD90 antibodies disclosed in US Patent Application No. US20210054068; and PCT Application No. WO2017214050.

In some embodiments, the target molecule is CD164. In some embodiments, the target cell is CD164+. In some embodiments, the targeting agent is an anti-CD164 antibody. Exemplary anti-CD164 antibodies include 67D2, H-4, 32G1, EML2058, 5C5, N6B6, 4B4, and 15-11-14; and anti-CD164 antibodies disclosed in PCT Application No. WO2006002438; and German Patent Nos. DE19727813C1 and DE19727815C1.

In some embodiments, the target molecule is CD49f. In some embodiments, the target cell is CD49f+. In some embodiments, the targeting agent is an anti-CD49f antibody. Exemplary anti-CD49f antibodies include CL6957, GoH3, SR45-00, and MP4F₁₀; and anti-CD49f antibodies disclosed in U.S. Pat. Nos. 5,538,725, 10,030,071; US Patent Application Nos. US20110301227, US20160194400, US20160280789; and PCT Application Nos. WO2015034052 and WO2018127655.

In some embodiments, the target molecule is CD146. In some embodiments, the target cell is CD146+. In some embodiments, the targeting agent is an anti-CD146 antibody. Exemplary anti-CD146 antibodies include imaprelimab, PRX003, ABX-MA1, huAA98, M2H-1, M2J-1, and JM1-24-3; and anti-CD146 antibodies disclosed in U.S. Pat. Nos. 6,924,360, 7,067,131, U.S. Pat. No. 709,844, U.S. Pat. Nos. 9,447,190, 9,782,500, 10,407,506, 10,414,825, 10,407,507, 10,584,177, 10,905,771, 11,427,648; US Patent Application Nos. US20030147809, US20040115205, US20060246077, US20140314744, US20150239980, US20140227292, US20160206764, US20190192573, US20170002089, US20150259419, US20170037144, US20170129954, US20170101470, US20180271994, US20180105602, US20200010563, US20200165336, US20200216560, US20200262929, US20200100838, US20220041748, US20220041749; and PCT Application Nos. WO2003057006, WO2003057837, WO2003057838, WO2012170071, WO2014000338, WO2015044218, WO2015136469, WO2015136470, WO2017046776, WO2017046774, WO2017149513, WO2017153953, WO2017208210, WO2018033630, WO2018220467, WO2018223140, WO2019068842, WO2019133639, WO2019137309, WO2020132190, WO2020132232, and WO2022082073.

Further exemplary targeting agents and corresponding target molecules are described in the table below. The targeting agent can be any described in the referenced associated documents that bind to the associated target molecule.

TABLE 3
Exemplary targeting agents and corresponding target molecules

| | | Associated | |
|---|---|---|---|
| Target Molecule | Targeting Agent | Documents | Company/Institution |
| CD117 (c-Kit) | N/A | WO2022187050 | Biolegend |
| CD117 (c-Kit) | Briquilimab (also known | US20130288303 | Amgen/Jasper Therapeutics |
| | as JSP191 or AMG191) | U.S. Pat. No. 8,791,249 | |
| | | US20110223165 | |
| | | U.S. Pat. No. 8,436,150 | |
| | | WO2007127317 | |
| | | U.S. Pat. No. 7,915,391 | |
| CD117 (c-Kit) | CDX-0158 (also known as | US20200385483 | Celldex |
| | KTN0158) | US20220010027 | |
| | | U.S. Pat. No. 10,774,146 | |
| | | US20190153094 | |
| | | US20190106510 | |
| | | U.S. Pat. No. 10,781,267 | |
| | | US20190100598 | |
| | | U.S. Pat. No. 10,793,639 | |
| | | US20170158778 | |
| | | U.S. Pat. No. 10,184,007 | |
| | | US20170073422 | |
| | | U.S. Pat. No. 10,189,907 | |
| | | US20160311924 | |
| | | U.S. Pat. No. 9,605,081 | |
| | | WO2015179737 | |
| | | US20170121408 | |
| | | U.S. Pat. No. 10,239,943 | |
| | | WO2015112822 | |
| | | WO2015050959 | |
| | | WO2014018625 | |
| | | US20140065168 | |
| | | U.S. Pat. No. 9,334,332 | |
| | | WO2012103165 | |
| | | US20140056905 | |
| | | U.S. Pat. No. 9,540,443 | |
| | | WO2011119948 | |
| | | US20130011406 | |
| CD117 (c-Kit) | CDX-0159 | WO2022159737 | Celldex |
| CD117 (c-Kit) | | US20220127358 | Forty Seven |
| | | US20210122822 | |
| | | U.S. Pat. No. 11,041,022 | |
| | | WO2020242895 | |
| | | US20200165337 | |
| | | WO2020112687 | |
| | | U.S. Pat. No. 11,208,482 | |
| CD117 (c-Kit) | | WO2021099418 | IDD-Tech |
| CD117 (c-Kit) | | WO2012154861 | ImClone |
| | | U.S. Pat. No. 8,552,157 | |
| CD117 (c-Kit) | | WO2015067667 | INSERM |
| | | US20160264680 | |
| | | U.S. Pat. No. 9,932,410 | |
| CD117 (c-Kit) | LOP628 (also known as | US20220305134 | Novartis |
| | LMJ729) | US20200405875 | |
| | | US20200129632 | |
| | | WO2019244082 | |
| | | US20210228731 | |
| | | WO2018116178 | |
| | | US20190321483 | |
| | | U.S. Pat. No. 11,357,864 | |
| | | US20180125996 | |
| | | US20170035905 | |
| | | U.S. Pat. No. 10,117,953 | |
| | | WO2016020791 | |
| | | US20180193475 | |
| | | U.S. Pat. No. 10,786,578 | |
| | | US20150320880 | |
| | | US20170021033 | |
| | | US20140271688 | |
| | | WO2014150937 | |
| | | US20160030594 | |
| | | U.S. Pat. No. 9,498,543 | |
| | | U.S. Pat. No. 9,789,203 | |
| CD117 (c-Kit) | MGTA-117 (also known | US20220177575 | Magenta Thera |
| | as CD117-ADC or | US20220177578 | |
| | MGTA-C100) | US20220177577 | |
| | | US20220175944 | |
| | | US20220226493 | |
| | | US20210283267 | |
| | | US20210317228 | |
| | | US20200255523 | |
| | | WO2020219964 | |
| | | US20200376135 | |
| | | WO2020219748 | |
| | | WO2020219770 | |
| | | WO2020219774 | |
| | | WO2020219778 | |
| | | WO2020219775 | |
| | | WO2019234694 | |
| | | WO2019084067 | |
| | | WO2019084064 | |
| | | WO2019084057 | |
| | | WO2019084053 | |
| | | US20190153114 | |
| | | U.S. Pat. No. 10,882,915 | |
| | | U.S. Pat. No. 10,899,843 | |
| | | US20190134217 | |
| CD117 (c-Kit) | NN2101 (also known | WO2020076105 | Ajou University |
| | as 2G4) | WO2021107566 | |
| | | US20210355212 | |
| CD117 (c-Kit) | N/A (internal name | WO2016033201 | Stanford University |
| | SR1) | US20170224737 | |
| | | U.S. Pat. No. 10,406,179 | |
| CD117 (c-Kit) | N/A | US20200283521 | Granular |
| | | U.S. Pat. No. 11,377,493 | Therapeutics/Ultrahuman |
| | | US20200010548 | Four |
| | | U.S. Pat. No. 10,611,838 | |
| | | WO2019155067 | |
| CD117 (c-Kit) | N/A | WO2021188590 | Magenta Thera |
| (CD117/CD3 | | | |
| bispecific) | | | |
| CD117 (c-Kit) | N/A | WO2021044008 | University of Zurich |
| (CD117/CD3 | | | |
| bispecific) | | | |
| CD110 (TPO | N/A | WO2011060076 | Amgen |
| Receptor/c-Mpl) | | US20120269814 | |
| CD110 (TPO | N/A (also known as | US20120015436 | Chugai Pharmaceutical |
| Receptor/c-Mpl) | TA136) | U.S. Pat. No. 8,586,039 | |
| | | US20090297501 | |
| | | U.S. Pat. No. 9,493,569 | |
| | | WO2006106903 | |
| | | U.S. Pat. No. 7,691,588 | |
| | | U.S. Pat. No. 8,034,903 | |
| CD110 (TPO | N/A | WO2022114603 | Daegu Gyeongbuk Institute |
| Receptor/c-Mpl) | | | of Science and Technology |
| CD110 (TPO | N/A | US20130053547 | Kyowa Hakko Kirin Co. |
| Receptor/c-Mpl) | | U.S. Pat. No. 8,999,329 | |
| | | US20120053326 | |
| | | U.S. Pat. No. 8,236,314 | |
| | | WO2009041734 | |
| | | US20100004429 | |
| | | U.S. Pat. No. 8,048,421 | |
| ITGA3 (Integrin alpha | N/A | US20190309072 | Cytomx Therapeutics |
| 3, VLA3A, CD49c) | | WO2016179335 | |
| | | US20160355592 | |
| | | U.S. Pat. No. 10,233,244 | |
| ITGA3 (Integrin alpha | N/A (also known as OV- | Ke et al. Cancer Sci. | Academia Sinica |
| 3, VLA3A, CD49c) | Ab 30-7) | 2020; 111: 3478- | |
| | | 3492. | |
| CD46 (MCP, | N/A | WO2021257542 | BioAtla |
| Membrane cofactor | | | |
| protein) | | | |
| CD46 (MCP, | FOR46 (also known as | WO2022150512 | Fortis Therapeutics Inc/ |
| Membrane cofactor | YS5FL) | WO2022150514 | University of California |
| protein) | | WO2022150517 | |
| | | WO2022032020 | |
| | | US20200199245 | |
| | | US20200040094 | |
| | | U.S. Pat. No. 11,208,493 | |
| | | WO2018089807 | |
| | | US20190276553 | |
| | | U.S. Pat. No. 11,434,301 | |
| | | US20170240643 | |
| | | U.S. Pat. No. 10,400,045 | |
| | | WO2016040683 | |
| | | US20170362330 | |
| | | U.S. Pat. No. 10,533,056 | |
| | | US20140271685 | |
| | | U.S. Pat. No. 9,567,402 | |
| CD46 (MCP, | N/A | WO2003032814 | MacroGenics Inc |
| Membrane cofactor | | | |
| protein) | | | |
| CD46 (MCP, | N/A | US20170081419 | StemCentRx |
| Membrane cofactor | | U.S. Pat. No. 10,017,565 | |
| protein) | | WO2012031273 | |
| | | U.S. Pat. No. 9,458,231 | |
| | | US20130302355 | |

### B. F Proteins

In some embodiments, the lipid particle comprises one or more paramyxovirus fusion (F) proteins. In some embodiments, the lipid particle contains an exogenous or overexpressed paramyxovirus fusion (F) protein. In some embodiments, the paramyxovirus fusion (F) protein is disposed in the lipid bilayer. In some embodiments, the paramyxovirus fusion (F) protein (e.g., fusogen) facilitates the fusion of the lipid particle to a membrane. In some embodiments, the membrane is a plasma cell membrane. In some embodiments, the paramyxovirus fusion (F) protein binds a binding partner on a target cell surface. In some embodiments, the paramyxovirus fusion (F) protein comprises a protein with a hydrophobic fusion peptide domain.

In some embodiments the paramyxovirus fusion (F) protein is or comprises a Nipah virus protein F, a measles virus F protein, a tupaia paramyxovirus F protein, a paramyxovirus F protein, a Hendra virus F protein, a Henipavirus F protein, a Morbillivirus F protein, a respirovirus F protein, a Sendai virus F protein, a rubulavirus F protein, or an avulavirus F protein.

In some embodiments, the paramyxovirus fusion (F) protein comprises a henipavirus F protein molecule or biologically active portion thereof. In some embodiments, the Henipavirus F protein is a Hendra (HeV) virus F protein, a Nipah (NiV) virus F-protein, a Cedar (CedPV) virus F protein, a Mòjiang virus F protein, a Langya virus F protein or a bat Paramyxovirus F protein or a biologically active portion thereof.

Table 4 provides non-limiting examples of F proteins. In some embodiments, the N-terminal hydrophobic fusion peptide domain of the F protein molecule or biologically active portion thereof is exposed on the outside of lipid bilayer.

In some embodiments, the paramyxovirus fusion (F) protein is a variant Nipah F protein (NiV-F). In some embodiments, the variant NiV-F protein exhibits fusogenic activity. In some embodiments, the variant NiV-F facilitates the fusion of the lipid particle (e.g. lentiviral vector) to a membrane. F proteins of henipaviruses, including NiV-F, are encoded as F0 precursors containing a signal peptide (e.g. corresponding to amino acid residues 1-26 of the below). Following cleavage of the signal peptide, the mature F0 (SEQ ID NO:235 lacking the signal peptide, i.e. set forth in SEQ ID NO: 256) is transported to the cell surface, then endocytosed and cleaved by cathepsin L (e.g. between amino acids 109-110 of NiV-F corresponding to amino acids set forth in SEQ ID NO:235) into the mature fusogenic subunits F₁ (e.g. corresponding to amino acids 110-546 of NiV-F set forth in SEQ ID NO: 235) and F₂ (e.g. corresponding to amino acid residues 27-109 of NiV-F set forth in SEQ ID NO: 235). The F₁ and F₂ subunits are associated by a disulfide bond and recycled back to the cell surface. The F₁ subunit contains the fusion peptide domain located at the N terminus of the F₁ subunit (e.g. corresponding to amino acids 110-129 of the below e.g. NiV-F set forth in SEQ ID NO:235) where it is able to insert into a cell membrane to drive fusion. In particular cases, fusion activity is blocked by association of the F protein with G protein, until G engages with a target molecule resulting in its disassociation from F and exposure of the fusion peptide to mediate membrane fusion.

Among different henipavirus species, the sequence and activity of the F protein is highly conserved. For examples, the F protein of NiV and HeV viruses share 89% amino acid sequence identity. Further, in some cases, the henipavirus F proteins exhibit compatibility with G proteins from other species to trigger fusion (Brandel-Tretheway et al. Journal of Virology. 2019. 93 (13): e00577-19). In some aspects of the provided lipid particles, the F protein is heterologous to the G protein, i.e. the F and G protein or biologically active portions are from different henipavirus species. For example, the F protein is from Hendra virus and the G protein is from Nipah virus. In other aspects, the F protein can be a chimeric F protein containing regions of F proteins from different species of Henipavirus. In some embodiments, switching a region of amino acid residues of the F protein from one species of Henipavirus to another can result in fusion to the G protein of the species comprising the amino acid insertion. (Brandel-Tretheway et al. 2019). In some cases, the chimeric F protein contains an extracellular domain from one henipavirus species and a transmembrane and/or cytoplasmic domain from a different henipavirus species. For example, the F protein contains an extracellular domain of Hendra virus and a transmembrane/cytoplasmic domain of Nipah virus. F protein sequences disclosed herein are predominantly disclosed as expressed sequences including an N-terminal signal sequence. As such N-terminal signal sequences are commonly cleaved co- or post-translationally, the mature protein sequences for all F protein sequences disclosed herein are also contemplated as lacking the N-terminal signal sequence.

TABLE 4
Non-limiting Examples of F Proteins

| | | | | | SEQ ID |
|---|---|---|---|---|---|
| | | | | | NO |
| | | Full | | | (without |
| Genbank | Nucleotides | Gene | | SEQ | signal |
| ID | of CDS | Name | Sequence | ID | sequence) |
| AF017149 | 6618-8258 | gb:AF017149\| | MATQEVRLKCLLCGIIVLVLSLEGLGILH | 234 | 255 |
| | | Organism: | YEKLSKIGLVKGITRKYKIKSNPLTKDIVI | | |
| | | Hendra | KMIPNVSNVSKCTGTVMENYKSRLTGILS | | |
| | | virus\|Strain | PIKGAIELYNNNTHDLVGDVKLAGVVMA | | |
| | | Name:UNKNOWN- | GIAIGIATAAQITAGVALYEAMKNADNIN | | |
| | | AF017149\| | KLKSSIESTNEAVVKLQETAEKTVYVLTA | | |
| | | Protein Name: | LODYINTNLVPTIDQISCKQTELALDLALS | | |
| | | fusion\|Gene | KYLSDLLFVFGPNLQDPVSNSMTIQAISQ | | |
| | | Symbol:F | AFGGNYETLLRTLGYATEDFDDLLESDSI | | |
| | | | AGQIVYVDLSSYYIIVRVYFPILTEIQQAY | | |
| | | | VQELLPVSFNNDNSEWISIVPNFVLIRNTL | | |
| | | | ISNIEVKYCLITKKSVICNQDYATPMTAS | | |
| | | | VRECLTGSTDKCPRELVVSSHVPRFALSG | | |
| | | | GVLFANCISVTCQCQTTGRAISQSGEQTL | | |
| | | | LMIDNTTCTTVVLGNIIISLGKYLGSINYN | | |
| | | | SESIAVGPPVYTDKVDISSQISSMNQSLQQ | | |
| | | | SKDYIKEAQKILDTVNPSLISMLSMIILYV | | |
| | | | LSIAALCIGLITFISFVIVEKKRGNYSRLDD | | |
| | | | RQVRPVSNGDLYYIGT | | |
| Q9IH63 | | Additional | MVVILDKRCYCNLLILILMISECSVGILHY | 235 | 256 |
| | | in cluster: | EKLSKIGLVKGVTRKYKIKSNPLTKDIVIK | | |
| | | sp\|Q9IH63\| | MIPNVSNMSQCTGSVMENYKTRLNGILT | | |
| | | FUS_NIPAV | PIKGALEIYKNNTHDLVGDVRLAGVIMA | | |
| | | Fusion | GVAIGIATAAQITAGVALYEAMKNADNI | | |
| | | glycoprotein | NKLKSSIESTNEAVVKLQETAEKTVYVLT | | |
| | | F0OS = Nipah | ALQDYINTNLVPTIDKISCKQTELSLDLAL | | |
| | | virus | SKYLSDLLFVFGPNLQDPVSNSMTIQAIS | | |
| | | | QAFGGNYETLLRTLGYATEDFDDLLESD | | |
| | | | SITGQIIYVDLSSYYIIVRVYFPILTEIQQA | | |
| | | | YIQELLPVSFNNDNSEWISIVPNFILVRNT | | |
| | | | LISNIEIGFCLITKRSVICNQDYATPMTNN | | |
| | | | MRECLTGSTEKCPRELVVSSHVPRFALSN | | |
| | | | GVLFANCISVTCQCQTTGRAISQSGEQTL | | |
| | | | LMIDNTTCPTAVLGNVIISLGKYLGSVNY | | |
| | | | NSEGIAIGPPVFTDKVDISSQISSMNQSLQ | | |
| | | | QSKDYIKEAQRLLDTVNPSLISMLSMIILY | | |
| | | | VLSIASLCIGLITFISFIIVEKKRNTYSRLED | | |
| | | | RRVRPTSSGDLYYIGT | | |
| JQ001776 | 6129-8166 | gb:JQ001776: | MSNKRTTVLIIISYTLFYLNNAAIVGFDFD | 236 | 257 |
| | | 6129-8166\| | KLNKIGVVQGRVLNYKIKGDPMTKDLVL | | |
| | | Organism: | KFIPNIVNITECVREPLSRYNETVRRLLLPI | | |
| | | Cedar virus\| | HNMLGLYLNNTNAKMTGLMIAGVIMGG | | |
| | | Strain Name: | IAIGIATAAQITAGFALYEAKKNTENIQKL | | |
| | | CG1a\|Protein | TDSIMKTQDSIDKLTDSVGTSILILNKLQT | | |
| | | Name:fusion | YINNQLVPNLELLSCRQNKIEFDLMLTKY | | |
| | | glycoprotein\| | LVDLMTVIGPNINNPVNKDMTIQSLSLLF | | |
| | | Gene Symbol: | DGNYDIMMSELGYTPQDFLDLIESKSITG | | |
| | | F | QIIYVDMENLYVVIRTYLPTLIEVPDAQIY | | |
| | | | EFNKITMSSNGGEYLSTIPNFILIRGNYMS | | |
| | | | NIDVATCYMTKASVICNQDYSLPMSQNL | | |
| | | | RSCYQGETEYCPVEAVIASHSPRFALTNG | | |
| | | | VIFANCINTICRCQDNGKTITQNINQFVSM | | |
| | | | IDNSTCNDVMVDKFTIKVGKYMGRKDIN | | |
| | | | NINIQIGPQIIIDKVDLSNEINKMNQSLKDS | | |
| | | | IFYLREAKRILDSVNISLISPSVQLFLIIISVL | | |
| | | | SFIILLIIIVYLYCKSKHSYKYNKFIDDPDY | | |
| | | | YNDYKRERINGKASKSNNIYYVGD | | |
| NC_025352 | 5950-8712 | gb:NC_025352: | MALNKNMFSSLFLGYLLVYATTVQSSIH | 237 | 258 |
| | | 5950-8712\| | YDSLSKVGVIKGLTYNYKIKGSPSTKLM | | |
| | | Organism: | VVKLIPNIDSVKNCTQKQYDEYKNLVRK | | |
| | | Mojiang | ALEPVKMAIDTMLNNVKSGNNKYRFAG | | |
| | | virus\|Strain | AIMAGVALGVATAATVTAGIALHRSNEN | | |
| | | Name: | AQAIANMKSAIQNTNEAVKQLQLANKQT | | |
| | | Tongguan1\| | LAVIDTIRGEINNNIIPVINQLSCDTIGLSV | | |
| | | Protein Name: | GIRLTQYYSEIITAFGPALQNPVNTRITIQA | | |
| | | fusion | ISSVFNGNFDELLKIMGYTSGDLYEILHSE | | |
| | | protein\|Gene | LIRGNIIDVDVDAGYIALEIEFPNLTLVPN | | |
| | | Symbol:F | AVVQELMPISYNIDGDEWVTLVPRFVLT | | |
| | | | RTTLLSNIDTSRCTITDSSVICDNDYALPM | | |
| | | | SHELIGCLQGDTSKCAREKVVSSYVPKFA | | |
| | | | LSDGLVYANCLNTICRCMDTDTPISQSLG | | |
| | | | ATVSLLDNKRCSVYQVGDVLISVGSYLG | | |
| | | | DGEYNADNVELGPPIVIDKIDIGNQLAGI | | |
| | | | NQTLQEAEDYIEKSEEFLKGVNPSIITLGS | | |
| | | | MVVLYIFMILIAIVSVIALVLSIKLTVKGN | | |
| | | | VVRQQFTYTQHVPSMENINYVSH | | |
| NC_025256 | 6865-8853 | gb:NC_025256: | MKKKTDNPTISKRGHNHSRGIKSRALLRE | 238 | 259 |
| | | 6865-8853\| | TDNYSNGLIVENLVRNCHHPSKNNLNYT | | |
| | | Organism:Bat | KTQKRDSTIPYRVEERKGHYPKIKHLIDK | | |
| | | Paramyxovirus | SYKHIKRGKRRNGHNGNIITIILLLILILKT | | |
| | | Eid_hel/GH- | QMSEGAIHYETLSKIGLIKGITREYKVKG | | |
| | | M74a/GHA/ | TPSSKDIVIKLIPNVTGLNKCTNISMENYK | | |
| | | 2009\|Strain | EQLDKILIPINNIIELYANSTKSAPGNARFA | | |
| | | Name:BatPV/ | GVIIAGVALGVAAAAQITAGIALHEARQN | | |
| | | Eid_hel/GH- | AERINLLKDSISATNNAVAELQEATGGIV | | |
| | | M74a/GHA/ | NVITGMQDYINTNLVPQIDKLQCSQIKTA | | |
| | | 2009\|Protein | LDISLSQYYSEILTVFGPNLQNPVTTSMSI | | |
| | | Name:fusion | QAISQSFGGNIDLLLNLLGYTANDLLDLL | | |
| | | protein\|Gene | ESKSITGQITYINLEHYFMVIRVYYPIMTTI | | |
| | | Symbol:F | SNAYVQELIKISFNVDGSEWVSLVPSYILI | | |
| | | | RNSYLSNIDISECLITKNSVICRHDFAMPM | | |
| | | | SYTLKECLTGDTEKCPREAVVTSYVPRFA | | |
| | | | ISGGVIYANCLSTTCQCYQTGKVIAQDGS | | |
| | | | QTLMMIDNQTCSIVRIEEILISTGKYLGSQ | | |
| | | | EYNTMHVSVGNPVFTDKLDITSQISNINQ | | |
| | | | SIEQSKFYLDKSKAILDKINLNLIGSVPISI | | |
| | | | LFIIAILSLILSIITFVIVMIIVRRYNKYTPLI | | |
| | | | NSDPSSRRSTIQDVYIIPNPGEHSIRSAARS | | |
| | | | IDRDRD | | |

In some embodiments, the F protein or the biologically active portion thereof is a wild-type Nipah virus F (NiV-F) protein or a Hendra virus F protein or is a functionally active variant or biologically active portion thereof. For instance, in some embodiments, the F protein or the biologically active portion thereof is a wild-type NiV-F protein or a functionally active variant or a biologically active portion thereof.

In some embodiments, the F protein has the sequence of amino acids set forth in SEQ ID NO: 234, SEQ ID NO:235, SEQ ID NO:236, SEQ ID NO:237, or SEQ ID NO:238, or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 234, SEQ ID NO:235, SEQ ID NO:236, SEQ ID NO:237, or SEQ ID NO:238, and retains fusogenic activity in conjunction with a G protein, such as a variant NiV-G as provided herein. In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:234, SEQ ID NO:235, SEQ ID NO:236, SEQ ID NO:237, or SEQ ID NO:238.

In particular embodiments, the F protein has the sequence of amino acids set forth in SEQ ID NO:255, SEQ ID NO:256, SEQ ID NO:257, SEQ ID NO:258, or SEQ ID NO:259, or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 255, SEQ ID NO:256, SEQ ID NO:257, SEQ ID NO:258, or SEQ ID NO:259, and retains fusogenic activity in conjunction with a G protein, such as a variant NiV-G as provided herein. In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:255, SEQ ID NO:256, SEQ ID NO:257, SEQ ID NO:258, or SEQ ID NO:259.

Fusogenic activity includes the activity of the paramyxovirus fusion (F) protein in conjunction with a paramyxovirus envelope protein (e.g., G protein or G proteins) to promote or facilitate fusion of two membrane lumens, such as the lumen of the targeted lipid particle having embedded in its lipid bilayer a henipavirus F and at least two G proteins, and a cytoplasm of a target cell, e.g. a cell that contains a surface receptor or molecule that is recognized or bound by the targeted envelope protein. In some embodiments, the F protein and at least one G protein are from the same Henipavirus species (e.g. NiV-G and NiV-F). In some embodiments, the F protein and at least one G protein are from different Henipavirus species (e.g. NiV-G and HeV-F). In particular embodiments, the F protein of the functionally active variant or biologically active portion retains the cleavage site cleaved by cathepsin L (e.g. corresponding to the cleavage site between amino acids 109-110 of SEQ ID NO:235).

Reference to retaining fusogenic activity includes activity (in conjunction with a G protein, such as a variant G protein provided herein) that is between at or about 10% and at or about 150% or more of the level or degree of binding of the corresponding wild-type F protein, such as set forth in SEQ ID NO: 234, SEQ ID NO:235, SEQ ID NO:236, SEQ ID NO:237, or SEQ ID NO:238, SEQ ID NO:255, SEQ ID NO:256, SEQ ID NO:257, SEQ ID NO:258, or SEQ ID NO:259 or a cathepsin L cleaved from thereof containing an F1 and F2 subunit. In some embodiments, the fusogenic activity is at least or at least about 10% of the level or degree of fusogenic activity of the corresponding wild-type F protein, such as at least or at least about 15% of the level or degree of fusogenic activity of the corresponding wild-type F protein, such as at least or at least about 20% of the level or degree of fusogenic activity of the corresponding wild-type F protein, such as at least or at least about 25% of the level or degree of fusogenic activity of the corresponding wild-type F protein, such as at least or at least about 30% of the level or degree of fusogenic activity of the corresponding wild-type F protein, such as at least or at least about 35% of the level or degree of fusogenic activity of the corresponding wild-type F protein, such as at least or at least about 40% of the level or degree of fusogenic activity of the corresponding wild-type F protein, such as at least or at least about 45% of the level or degree of fusogenic activity of the corresponding wild-type F protein, such as at least or at least about 50% of the level or degree of fusogenic activity of the corresponding wild-type F protein, such as at least or at least about 55% of the level or degree of fusogenic activity of the corresponding wild-type F protein, such as at least or at least about 60% of the level or degree of fusogenic activity of the corresponding wild-type F protein, such as at least or at least about 65% of the level or degree of fusogenic activity of the corresponding wild-type F protein, such as at least or at least about 70% of the level or degree of fusogenic activity of the corresponding wild-type F protein, such as at least or at least about 75% of the level or degree of fusogenic activity of the corresponding wild-type F protein, such as at least or at least about 80% of the level or degree of fusogenic activity of the corresponding wild-type F protein, such as at least or at least about 85% of the level or degree of fusogenic activity of the corresponding wild-type F protein, such as at least or at least about 90% of the level or degree of fusogenic activity of the corresponding wild-type F protein, such as at least or at least about 95% of the level or degree of fusogenic activity of the corresponding wild-type F protein, such as at least or at least about 100% of the level or degree of fusogenic activity of the corresponding wild-type F protein, or such as at least or at least about 120% of the level or degree of fusogenic activity of the corresponding wild-type F protein.

In some embodiments, the paramyxovirus fusion (F) protein is a mutant F protein that is a functionally active fragment or a biologically active portion containing one or more amino acid mutations, such as one or more amino acid insertions, deletions, substitutions or truncations. In some embodiments, the mutations described herein relate to amino acid insertions, deletions, substitutions or truncations of amino acids compared to a reference F protein sequence. In some embodiments, the reference F protein sequence is the wild-type sequence of an F protein or a biologically active portion thereof. In some embodiments, the mutant F protein or the biologically active portion thereof is a mutant of a wild-type Hendra (Hev) virus F protein, a Nipah (NiV) virus F-protein, a Cedar (CedPV) virus F protein, a Mojiang virus F protein or a bat Paramyxovirus F protein. In some embodiments, the wild-type F protein is encoded by a sequence of nucleotides that encodes any one of SEQ ID NO:234, SEQ ID NO:235, SEQ ID NO:236, SEQ ID NO:237, or SEQ ID NO:238, SEQ ID NO:255, SEQ ID NO: 256, SEQ ID NO:257, SEQ ID NO:258, or SEQ ID NO:259 or a cathepsin L cleaved from thereof containing an F1 and F2 subunit.

In some embodiments, the mutant F protein is a biologically active portion that is truncated and lacks up to 22 contiguous amino acid residues at or near the C-terminus of the wild-type F protein, such as a wild-type F protein set forth in any one of SEQ ID NO:234, SEQ ID NO:235, SEQ ID NO:236, SEQ ID NO:237, or SEQ ID NO:238, SEQ ID NO:255, SEQ ID NO:256, SEQ ID NO:257, SEQ ID NO: 258, or SEQ ID NO:259. In some embodiments, the mutant F protein is truncated and lacks up to 22 contiguous amino acids, such as up to 21, 20, 19, 18, 17, 16, 15, 14, 13, 12, 11, 10, 9, 8, 7, 6, 5, 4, 3, 2, or 1 contiguous amino acids at the C-terminus of the wild-type F protein.

In some embodiments, the NiV-F, such as a mutant or truncated NiV-F, of a provided lipid particle includes the F0 precursor or a proteolytically cleaved form thereof containing the F1 and F2 subunits, such as resulting following proteolytic cleavage at the cleavage site (e.g. between amino acids corresponding to amino acids between amino acids 109-110 of SEQ ID NO:235) to produce two chains that can be linked by disulfide bond. In some embodiments, the NiV-F, such as wild-type NiV-F or a truncated or mutated NiV-F protein, is produced or encoded as an F0 precursor which then is able to be proteolytically cleaved to result in an F protein containing the F1 and F2 subunit linked by a disulfide bond. Hence, it is understood that reference to a particular sequence (SEQ ID NO) of a NiV-F herein is typically with reference to the F0 precursor sequence but also is understood to include the proteolytically cleaved form or sequence thereof containing the two cleaved chains, F1 and F2. For instance, the NiV-F, such as a mutant or truncated NiV-F, contains an F1 subunit corresponding to amino acids 110-546 of NiV-F set forth in SEQ ID NO:235 or truncated or mutant sequence thereof, and an F2 corresponding to amino acid residues 27-109 of NiV-F set forth in SEQ ID NO: 235.

In some embodiments, the mutant F protein is a biologically active portion that is truncated and lacks up to 22 contiguous amino acid residues at or near the C-terminus of the wild-type NiV-F protein, such as a wild-type NiV-F protein set forth in SEQ ID NO:235 or SEQ ID NO:256. In some embodiments, the mutant F protein is truncated and lacks up to 22 contiguous amino acids, such as up to 21, 20, 19, 18, 17, 16, 15, 14, 13, 12, 11, 10, 9, 8, 7, 6, 5, 4, 3, 2, or 1 contiguous amino acids at the C-terminus of the wild-type NiV-F protein, such as a wild-type NiV-F protein set forth in SEQ ID NO:235 or SEQ ID NO:256. In some embodiments, the mutant F protein contains an F1 subunit and an F2 subunit in which (1) the F1 subunit is truncated and lacks up to 22 contiguous amino acids at or near the C-terminus of the wild-type F1 subunit, such as lacks up to 22 contiguous amino acids at or near the C-terminus of the wild-type F1 subunit corresponding to amino acids 110-546 of NiV-F set forth in SEQ ID NO:235, and (2) the F2 subunit has the sequence corresponding to amino acid residues 27-109 of NiV-F set forth in SEQ ID NO:235.

In some embodiments, the paramyxovirus fusion (F) protein is a mutant NiV-F protein that is a biologically active portion thereof that comprises a 22 amino acid truncation at or near the C-terminus of the wild-type NiV-F protein (SEQ ID NO:235 or SEQ ID NO:256). In some embodiments, the NiV-F protein is encoded by a nucleotide sequence that encodes the sequence set forth in SEQ ID NO: 226. In some embodiments, the NiV-F proteins is encoded by a nucleotide sequence that encodes sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 226. In particular embodiments, the variant F protein is a mutant NiV-F protein that has the sequence of amino acids set forth in SEQ ID NO:227. In some embodiments, the NiV-F proteins is encoded by a sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 227. In some embodiments, the F protein molecule or biologically active portion thereof comprises the sequence set forth in SEQ ID NO: 227.

In some embodiments, the mutant F protein contains an F1 subunit and an F2 subunit in which (1) the F1 subunit is set forth as amino acids 110-524 of SEQ ID NO:226, and (2) the F2 subunit is set forth as amino acids 27-109 of SEQ ID NO:226.

In some embodiments, the mutant F protein contains an F1 subunit and an F2 subunit in which (1) the F1 subunit is set forth as amino acids 84-498 of SEQ ID NO:227, and (2) the F2 subunit is set forth as amino acids 1-83 of SEQ ID NO:227.

### C. Polynucleotides

Provided herein are polynucleotides comprising a nucleic acid sequence encoding a retargeted attachment protein. Also provided herein are polynucleotides encoding at least two retargeted attachment proteins. In some embodiments, the polynucleotides comprise a nucleic acid sequence encoding a G protein, F protein, or biologically active portion thereof. In some embodiments, the polynucleotides comprise a nucleic acid sequence encoding a first G protein, and a second G protein, or biologically active portion thereof. In some embodiments, the polynucleotides comprise a nucleic acid sequence encoding a first G protein, a second G protein, an F protein, or biologically active portion thereof. In some embodiments, the polynucleotides comprise a nucleic acid sequence encoding a first G protein, a second G protein, a third G protein, an F protein, or biologically active portion thereof. In some embodiments, the polynucleotides comprise a nucleic acid sequence encoding a first G protein, a second G protein, a third G protein, a fourth G protein, an F protein, or biologically active portion thereof. In some embodiments, the polynucleotides comprise a nucleic acid sequence encoding a first G protein, a second G protein, a third G protein, a fourth G protein, a fifth G protein, an F protein, or biologically active portion thereof. In some embodiments, the polynucleotides comprise a nucleic acid sequence encoding a first G protein, a second G protein, a third G protein, a fourth G protein, a fifth G protein, one or more additional G proteins, an F protein, or biologically active portion thereof. In some embodiments, the polynucleotides further comprise a nucleic acid sequence encoding a binding domain, such as single domain antibody (sdAb) variable domain or biologically active portion thereof. The polynucleotides may include a sequence of nucleotides encoding any of the chimeric attachment described above. The polynucleotide can be a synthetic nucleic acid. Also provided are expression vector containing any of the provided polynucleotides.

In some of any embodiments, expression of natural or synthetic nucleic acids is typically achieved by operably linking a nucleic acid encoding the gene of interest to a promoter and incorporating the construct into an expression vector. In some embodiments, vectors can be suitable for replication and integration in eukaryotes. In some embodiments, cloning vectors contain transcription and translation terminators, initiation sequences, and promoters useful for expression of the desired nucleic acid sequence. In some of any embodiments, a plasmid comprises a promoter suitable for expression in a cell.

In some embodiments, the polynucleotides contain at least one promoter that is operatively linked to control expression of the targeted retargeted attachment protein and/or G protein and/or F protein. For expression of the retargeted attachment protein, at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

In some embodiments, additional promoter elements, e.g., enhancers, regulate the frequency of transcriptional initiation. In some embodiments, additional promoter elements are located in the region 30-110 bp upstream of the start site, although a number of promoters have recently been shown to contain functional elements downstream of the start site as well. In some embodiments, spacing between promoter elements frequently is flexible, so that promoter function is preserved when elements are inverted or moved relative to one another. In some embodiments, the thymidine kinase (tk) promoter, the spacing between promoter elements can be increased to 50 bp apart before activity begins to decline. In some embodiments, depending on the promoter, individual elements can function either cooperatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein (U.S. Pat. Nos. 4,683,202 and 5,928,906).

In some embodiments, a suitable promoter is the immediate early cytomegalovirus (CMV) promoter sequence. In some embodiments, the promoter sequence is a strong constitutive promoter sequence capable of driving high levels of expression of any polynucleotide sequence operatively linked thereto. In some embodiments, a suitable promoter is Elongation Growth Factor-la (EF-1 a). In some embodiments, a suitable promoter is human PGK (hPGK). In some embodiments, a suitable promoter is RPBSA. In some embodiments, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

In some embodiments, the promoter is a promoter associated with T cells (e.g., a T cell promoter). In some embodiments, the promoter is a promoter that drives gene expression in T cells. In some embodiments, the promoter sequence is a T cells promoter sequence capable of driving high levels of expression of any polynucleotide sequence operatively linked thereto. In some embodiments, the promoter is dLck or CD38-promoter. In some embodiments, the promoter is ELAM promoter, hTNFAIP1, hVCAM1, hCCL2, or pA20 promoter.

In some embodiments, the promoter is an inducible promoter. In some embodiments, the inducible promoter provides a molecular switch capable of turning on expression of the polynucleotide sequence which it is operatively linked when such expression is desired, or turning off the expression when expression is not desired. In some embodiments, inducible promoters comprise metallothionine promoter, a glucocorticoid promoter, a progesterone promoter, and a tetracycline promoter.

In some embodiments, exogenously controlled inducible promoters can be used to regulate expression of the retargeted attachment protein, the G protein, the F protein, and/or an antigen binding domain such as a single domain antibody (sdAb) variable domain. For example, radiation-inducible promoters, heat-inducible promoters, and/or drug-inducible promoters can be used to selectively drive transgene expression in, for example, targeted regions. In such embodiments, the location, duration, and level of transgene expression can be regulated by the administration of the exogenous source of induction.

In some embodiments, expression of the retargeted attachment protein is regulated using a drug-inducible promoter. For example, in some cases, the promoter, enhancer, or transactivator comprises a Lac operator sequence, a tetracycline operator sequence, a galactose operator sequence, a doxycycline operator sequence, a rapamycin operator sequence, a tamoxifen operator sequence, or a hormone-responsive operator sequence, or an analog thereof. In some instances, the inducible promoter comprises a tetracycline response element (TRE). In some embodiments, the inducible promoter comprises an estrogen response element (ERE), which can activate gene expression in the presence of tamoxifen. In some instances, a drug-inducible element, such as a TRE, can be combined with a selected promoter to enhance transcription in the presence of drug, such as doxycycline. In some embodiments, the drug-inducible promoter is a small molecule-inducible promoter.

Any of the provided polynucleotides can be modified to remove CpG motifs and/or to optimize codons for translation in a particular species, such as human, canine, feline, equine, ovine, bovine, etc. species. In some embodiments, the polynucleotides are optimized for human codon usage (i.e., human codon-optimized). In some embodiments, the polynucleotides are modified to remove CpG motifs. In other embodiments, the provided polynucleotides are modified to remove CpG motifs and are codon-optimized, such as human codon-optimized. Methods of codon optimization and CpG motif detection and modification are well-known. Typically, polynucleotide optimization enhances transgene expression, increases transgene stability and preserves the amino acid sequence of the encoded polypeptide.

In order to assess the expression of the targeted envelope protein, the expression vector to be introduced into a cell can also contain either a selectable marker gene or a reporter gene or both to facilitate identification and selection of expressing particles, e.g. viral particles. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000, FEBS Lett. 479:79-82). Suitable expression systems are well known and may be prepared using well known techniques or obtained commercially. Internal deletion constructs may be generated using unique internal restriction sites or by partial digestion of non-unique restriction sites. Constructs may then be transfected into cells that display high levels of the desired polynucleotide and/or polypeptide expression. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

### II. LIPID PARTICLES AND METHODS OF PRODUCTION

Provided herein is a lipid particle comprising a lipid bilayer, a lumen surrounded by the lipid bilayer and a retargeted attachment protein, such as any as described, in which the retargeted attachment protein is embedded within the lipid bilayer. In some embodiments, the provided lipid particles preferentially target hematopoietic cells (e.g. T cells), which is mediated by the tropism of the retargeted attachment protein, such as a G protein. In some embodiments, the lipid particle may additionally contain an exogenous agent (e.g. therapeutic agent) for delivery to a cell. In some embodiments, a lipid particle is introduced to a cell in the subject. Also provided are methods of delivering any of the provided lipid particles to a cell.

In some embodiments, the provided lipid particles exhibit fusogenic activity, which is mediated by the retargeted attachment protein, such as a G protein and/or any of the provided F proteins that facilitates merger or fusion of the two lumens of the lipid particle and the target cell membranes. Thus, among provided lipid particles are fusosomes. In some embodiments, the fusosome comprises a naturally derived bilayer of amphipathic lipids with the retargeted attachment protein as a fusogen. In some embodiments, the fusosome comprises (a) a lipid bilayer, (b) a lumen (e.g., comprising cytosol) surrounded by the lipid bilayer; and (c) a fusogen that is exogenous or overexpressed relative to the source cell. In some embodiments, the retargeted attachment protein is disposed in the lipid bilayer. In some embodiments, the fusosome comprises several different types of lipids, e.g., amphipathic lipids, such as phospholipids

In some embodiments, the lipid particle includes a naturally derived bilayer of amphipathic lipids that encloses lumen or cavity. In some embodiments, the lipid particle comprises a lipid bilayer as the outermost surface. In some embodiments, the lipid bilayer encloses a lumen. In some embodiments, the lumen is aqueous. In some embodiments, the lumen is in contact with the hydrophilic head groups on the interior of the lipid bilayer. In some embodiments, the lumen is a cytosol. In some embodiments, the cytosol contains cellular components present in a source cell. In some embodiments, the cytosol does not contain components present in a source cell. In some embodiments, the lumen is a cavity. In some embodiments, the cavity contains an aqueous environment. In some embodiments, the cavity does not contain an aqueous environment.

In some aspects, the lipid bilayer is derived from a source cell during a process to produce a lipid-containing particle. Exemplary methods for producing lipid-containing particles are provided in Section I.E. In some embodiments, the lipid bilayer includes membrane components of the cell from which the lipid bilayer is produced, e.g., phospholipids, membrane proteins, etc. In some embodiments, the lipid bilayer includes a cytosol that includes components found in the cell from which the micro-vesicle is produced, e.g., solutes, proteins, nucleic acids, etc., but not all of the components of a cell, e.g., they lack a nucleus. In some embodiments, the lipid bilayer is considered to be exosome-like. The lipid bilayer may vary in size, and in some instances have a diameter ranging from 30 and 300 nm, such as from 30 and 150 nm, and including from 40 to 100 nm.

In some embodiments, the lipid bilayer is a viral envelope. In some embodiments, the viral envelope is obtained from a source cell. In some embodiments, the viral envelope is obtained by the viral capsid from the source cell plasma membrane. In some embodiments, the lipid bilayer is obtained from a membrane other than the plasma membrane of a host cell. In some embodiments, the viral envelope lipid bilayer is embedded with retargeted attachment proteins that are viral proteins, including viral glycoproteins as described herein such as a G protein and, in some aspects, also a F protein.

In other aspects, the lipid bilayer includes synthetic lipid complex. In some embodiments, the synthetic lipid complex is a liposome. In some embodiments, the lipid bilayer is a vesicular structure characterized by a phospholipid bilayer membrane and an inner aqueous medium. In some embodiments, the lipid bilayer has multiple lipid layers separated by aqueous medium. In some embodiments, the lipid bilayer forms spontaneously when phospholipids are suspended in an excess of aqueous solution. In some examples, the lipid components undergo self-rearrangement before the formation of closed structures and entrap water and dissolved solutes between the lipid bilayers.

In some embodiments, a targeted envelope protein and fusogen, such as any described above including any that are exogenous or overexpressed relative to the source cell, is disposed in the lipid bilayer.

In some embodiments, the lipid particle comprises several different types of lipids. In some embodiments, the lipids are amphipathic lipids. In some embodiments, the amphipathic lipids are phospholipids. In some embodiments, the phospholipids comprise phosphatidylcholine, phosphatidylethanolamine, phosphatidylinositol, and phosphatidylserine. In some embodiments, the lipids comprise phospholipids such as phosphocholines and phosphoinositols. In some embodiments, the lipids comprise DMPC, DOPC, and DSPC.

In some embodiments, the bilayer may be comprised of one or more lipids of the same or different type. In some embodiments, the source cell comprises a cell selected from HEK293 cells, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.

In some embodiments, the lipid particle can be a viral particle, a virus-like particle, a nanoparticle, a vesicle, an exosome, a dendrimer, a lentivirus, a viral vector, an enucleated cell, a microvesicle, a membrane vesicle, an extracellular membrane vesicle, a plasma membrane vesicle, a giant plasma membrane vesicle, an apoptotic body, a mitoparticle, a pyrenocyte, a lysosome, another membrane enclosed vesicle, or a lentiviral vector, a viral based particle, a virus like particle (VLP) or a cell based particle.

In particular embodiments, the lipid particle is virally derived. In some embodiments, the lipid particle can be a viral-based particle, such as a viral vector particle (e.g. lentiviral vector particle) or a virus-like particle (e.g. a lentiviral-like particle). In some embodiments, the lipid bilayer is a viral envelope. In some embodiments, the viral envelope is obtained from a host cell. In some embodiments, the viral envelope is obtained by the viral capsid from the source cell plasma membrane. In some embodiments, the lipid bilayer is obtained from a membrane other than the plasma membrane of a host cell. In some embodiments, the viral envelope lipid bilayer is embedded with viral proteins, including viral glycoproteins.

In particular embodiments, the lipid particle is not virally derived. In some embodiments, the lipid particle can be a nanoparticle, a vesicle, an exosome, a dendrimer, an enucleated cell, a microvesicle, a membrane vesicle, an extracellular membrane vesicle, a plasma membrane vesicle, a giant plasma membrane vesicle, an apoptotic body, a mitoparticle, a pyrenocyte, a lysosome, another membrane enclosed vesicle, or a cell derived particle.

In some embodiments, the lipid bilayer includes membrane components of the host cell from which the lipid bilayer is derived, e.g., phospholipids, membrane proteins, etc. In some embodiments, the lipid bilayer includes a cytosol that includes components found in the cell from which the vehicle is derived, e.g., solutes, proteins, nucleic acids, etc., but not all of the components of a cell, e.g., lacking a nucleus. In some embodiments, the lipid bilayer is considered to be exosome-like. The lipid bilayer may vary in size, and in some instances have a diameter ranging from 30 and 300 nm, such as from 30 and 150 nm, and including from 40 to 100 nm.

In particular embodiments, an exogenous agent, such as a polynucleotide or polypeptide, is encapsulated within the lumen of a lipid particle. Embodiments of provided lipid particles may have various properties that facilitate delivery of a payload, such as, e.g., a desired transgene or exogenous agent, to a target cell. The exogenous agent may be a polynucleotide or a polypeptide. In some embodiments, a lipid particle provided herein is administered to a subject, e.g., a mammal, e.g., a human. In such embodiments, the subject may be at risk of, may have a symptom of, or may be diagnosed with or identified as having, a particular disease or condition. In one embodiment, the subject has cancer. In one embodiment, the subject has an infectious disease. In some embodiments, the lipid particle contains nucleic acid sequences (polynucleotide) encoding an exogenous agent or a polypeptide exogenous agent for treating the disease or condition.

The lipid particles can include spherical particles or can include particles of elongated or irregular shape.

In some embodiments, a composition of particles can be assessed for one or more features related to their size, including diameter, range of variation thereof above and below an average (mean) or median value of the diameter, coefficient of variation, polydispersity index or other measure of size of particles in a composition. Various methods for particle characterization can be used, including, but not limited to, laser diffraction, dynamic light scattering (DLS; also known as photon correlation spectroscopy) or image analysis, such as microscopy or automated image analysis.

In some embodiments, the provided lipid particle has a diameter of, or the average (mean) diameter of particles in a composition is, less than about 3 μm, less than about 2 μm, less than about 1 μm, less than about 900 nm, less than about 800 nm, less than about 700 nm, less than about 600 nm, less than about 500 m, less than about 400 nm, less than about 300, less than about 200 nm, less than about 150 nm, less than about 100 nm, less than about 50 nm, or less than about 20 nm. In some embodiments, the lipid particle has a diameter of, or the average (mean) diameter of particles in a composition is, less than about 400 nm. In another embodiment, the lipid particle has a diameter of, or the average (mean) diameter of particles in a composition is, less than about 150 nm. In some embodiments, the lipid particle has a diameter of, or the average (mean) diameter of particles in a composition is, between at or about 2 μm and at or about 1 μm, between at or about 1 μm and at or about 900 nm, between at or about 900 nm and at or about 800 nm, between at or about 800 and at or about 700 nm, between at or about 700 nm and at or about 600 nm, between at or about 600 nm and at or about 500 nm, between at or about 500 nm and at or about 400 nm, between at or about 400 nm and at or about 300 nm, between at or about 300 nm and at or about 200 nm, between at or about 200 and at or about 100 nm, between at or about 100 and at or about 50 nm, or between at or about 20 nm and at or about 50 nm.

In some embodiments the median particle diameter in a composition of particles is between at or about 10 nm and at or about 1000 nM, between at or about 25 nm and at or about 500 nm, between at or about 40 nm and at or about 300 nm, between at or about 50 nm and at or about 250 nm, between at or about 60 nm and at or about 225 nm, between at or about 70 nm and at or about 200 nm, between at or about 80 nm and at or about 175 nm, or between at or about 90 nm and at or about 150 nm.

In some embodiments, 90% of the lipid particles in a composition fall within 50% of the median diameter of the lipid particles. In some embodiments, 90% of the lipid particles in a composition fall within 25% of the median diameter of the lipid particles. In some embodiments, 90% of the lipid particles in a composition fall within 20% of the median diameter. In some embodiments, 90% of the lipid particles in a composition fall within 15% of the median diameter of lipid particles. In some embodiments, 90% of the lipid particles in a composition fall within 10% of the median diameter of the lipid particles.

In some embodiments, 75% of the lipid particles in a composition fall within +/−2 or +/−1 St Dev standard deviations (St Dev) of the mean diameter of lipid particles. In some embodiments, 80% of the lipid particles in a composition fall within +/−2 St Dev or +/−1 St Dev of the mean diameter of lipid particles. In some embodiments, 85% of the lipid particles in a composition fall within +/−2 St Dev or +/−1 St Dev of the mean diameter of lipid particles. In some embodiments, 90% of the lipid particles in a composition fall within +/−2 St Dev or +/−1 St Dev of the mean diameter of lipid particles. In some embodiments, 95% of the lipid particles in a composition fall within +/−2 St Dev or +/−1 St Dev of the mean diameter of lipid particles.

In some embodiments, the lipid particles have an average hydrodynamic radius, e.g. as determined by DLS, of about 100 nm to about two microns. In some embodiments, the lipid particles have an average hydrodynamic radius between at or about 2 μm and at or about 1 μm, between at or about 1 μm and at or about 900 nm, between at or about 900 nm and at or about 800 nm, between at or about 800 and at or about 700 nm, between at or about 700 nm and at or about 600 nm, between at or about 600 nm and at or about 500 nm, between at or about 500 nm and at or about 400 nm, between at or about 400 nm and at or about 300 nm, between at or about 300 nm and at or about 200 nm, between at or about 200 and at or about 100 nm, between at or about 100 and at or about 50 nm, or between at or about 20 nm and at or about 50 nm.

In some embodiments, the lipid particles have an average geometric radius, e.g. as determined by a multi-angle light scattering, of about 100 nm to about two microns. In some embodiments, the lipid particles have an average geometric radius between at or about 2 μm and at or about 1 μm, between at or about 1 μm and at or about 900 nm, between at or about 900 nm and at or about 800 nm, between at or about 800 and at or about 700 nm, between at or about 700 nm and at or about 600 nm, between at or about 600 nm and at or about 500 nm, between at or about 500 nm and at or about 400 nm, between at or about 400 nm and at or about 300 nm, between at or about 300 nm and at or about 200 nm, between at or about 200 and at or about 100 nm, between at or about 100 and at or about 50 nm, or between at or about 20 nm and at or about 50 nm.

In some embodiments, the coefficient of variation (COV) (i.e. standard deviation divided by the mean) of a composition of lipid particles is less than at or about 30%, less than at or about 25%, less than at or about 20%, less than at or about 15%, less than at or about 10% or less than at or about 5%.

In some embodiment, provided compositions of lipid particles are characterized by their polydispersity index, which is a measure of the size distribution of the particles wherein values between 1 (maximum dispersion) and 0 (identical size of all of the particles) are possible. In some embodiments, compositions of lipid particles provided herein have a polydispersity index of between at or about 0.05 and at or about 0.7, between at or about 0.05 and at or about 0.6, between at or about 0.05 and at or about 0.5, between at or about 0.05 and at or about 0.4, between at or about 0.05 and at or about 0.3, between at or about 0.05 and at or about 0.2, between at or about 0.05 and at or about 0.1, between at or about 0.1 and at or about 0.7, between at or about 0.1 and at or about 0.6, between at or about 0.1 and at or about 0.5, between at or about 0.1 and at or about 0.4, between at or about 0.1 and at or about 0.3, between at or about 0.1 and at or about 0.2, between at or about 0.2 and at or about 0.7, between at or about 0.2 and at or about 0.6, between at or about 0.2 and at or about 0.5, between at or about 0.2 and at or about 0.4 between at or about 0.2 and at or about 0.3, between at or about 0.3 and at or about 0.7, between at or about 0.3 and at or about 0.6, between at or about 0.3 and at or about 0.5, between at or about 0.3 and at or about 0.4, between at or about 0.4 and at or about 0.7, between at or about 0.4 and at or about 0.6, between at or about 0.4 and at or about 0.5, between at or about 0.5 and at or about 0.7, between at or about 0.5 and at or about 0.6, or between at or about 0.6 and at or about 0.7. In some embodiments, the polydispersity index is less than at or about 0.05, less than at or about 0.1, less than at or about 0.15, less than at or about 0.2, less than at or about 0.25, less than at or about 0.3, less than at or about 0.4, less than at or about 0.5, less than at or about 0.6 or less than at or about 0.7. Various lipid particles are known, any of which can be generated in accord with the provided embodiments. Non-limiting examples of lipid particles include any as described in, or contain features as described in, International published PCT Application No. WO 2017/095946; WO 2017/095944; WO 2017/095940; WO 2019/157319; WO 2018/208728; WO 2019/113512; WO 2019/161281; WO 2020/102578; WO 2019/222403; WO 2020/014209; WO 2020/102485; WO 2020/102499; WO 2020/102503; WO 2013/148327; WO 2017/182585; WO 2011/058052; or WO 2017/068077, each of which are incorporated by reference in their entirety.

Features of the provided lipid particles are described in the following subsections.

### A. Viral-Based Particles

Provided herein are viral-based particles derived from a virus, including those derived from retroviruses or lentiviruses, containing a retargeted attachment protein, such as described in Section II. In some embodiments, the lipid particle's bilayer of amphipathic lipids is or comprises the viral envelope. In some embodiments, the lipid particle's bilayer of amphipathic lipids is or comprises lipids derived from a producer cell. In some embodiments, the viral envelope may comprise a fusogen, e.g., a fusogen that is endogenous to the virus or a pseudotyped fusogen. In some embodiments, the lipid particle's lumen or cavity comprises a viral nucleic acid, e.g., a retroviral nucleic acid, e.g., a lentiviral nucleic acid. In some embodiments, the viral nucleic acid may be a viral genome. In some embodiments, the lipid particle further comprises one or more viral non-structural proteins, e.g., in its cavity or lumen. In some embodiments, the viral-based particle is or comprises a virus-like particle (VLP). In some embodiments, the VLP does not comprise any viral genetic material. In some embodiments, the viral-based particle does not contain any virally derived nucleic acids or viral proteins, such as viral structural proteins.

Biological methods for introducing an exogenous agent to a host cell include the use of DNA and RNA vectors. DNA and RNA vectors can also be used to house and deliver polynucleotides and polypeptides. Viral vectors and virus like particles, and especially retroviral vectors, have become the most widely used method for inserting genes into mammalian, e.g., human cells. Other viral vectors and virus like particles can be derived from lentivirus, poxviruses, herpes simplex virus I, adenoviruses and adeno-associated viruses, and the like. See, for example, U.S. Pat. Nos. 5,350,674 and 5,585,362. Methods for producing cells comprising vectors and/or exogenous acids are well-known in the art. See, for example, Sambrook et al., 2001, Molecular Cloning: A Laboratory Manual, Cold Spring Harbor Laboratory, New York.

In some embodiments, the viral particles or virus-like particles bilayer of amphipathic lipids is or comprises lipids derived from an infected host cell. In some embodiments, the lipid bilayer is a viral envelope. In some embodiments, the viral particles or virus-like particles envelope is obtained from a host cell. In some embodiments, the viral particles or virus-like particles envelope is obtained by the viral capsid from the source cell plasma membrane. In some embodiments, the lipid bilayer is obtained from a membrane other than the plasma membrane of a host cell. In some embodiments, the viral particles or virus-like particles envelope lipid bilayer is embedded with retargeted attachment protein or proteins that are viral proteins, including viral glycoproteins, including a G protein and/or an F protein.

In some embodiments, one or more transducing units of viral particles or virus-like particles, e.g. retroviral particles or retroviral-like particles, are administered to the subject. In some embodiments, at least 1, 10, 100, 1000, 104, 105, 106, 107, 108, 109, 1010, 1011, 1012, 1013, or 1014, transducing units per kg are administered to the subject. In some embodiments at least 1, 10, 100, 1000, 104, 105, 106, 107, 108, 109, 1010, 1011, 1012, 1013, or 1014, transducing units per target cell per ml of blood are administered to the subject.

### 1. Viral Vector Particles

In some embodiments, the lipid particle is or comprises a virus or a viral vector, e.g., a retrovirus or retroviral vector, e.g., a lentivirus or lentiviral vector. In some embodiments, the virus or viral vector is recombinant. For instance, the viral particle may be referred to as a recombinant virus and/or a recombinant viral vector, which are used interchangeably. In some embodiments, the lipid particle is a recombinant lentivirus vector particle.

In some embodiments, a lipid particle comprises a lipid bilayer comprising a retroviral vector comprising an envelope. For instance, in some embodiments, the bilayer of amphipathic lipids is or comprises the viral envelope. The viral envelope may comprise a fusogen, e.g., retargeted attachment protein fusogen, that is endogenous to the virus or is a pseudotyped fusogen. In some embodiments, the viral vector's lumen or cavity comprises a viral nucleic acid, e.g., a retroviral nucleic acid, e.g., a lentiviral nucleic acid. The viral nucleic acid may be a viral genome. In some embodiments, the viral vector may further comprises one or more viral non-structural proteins, e.g., in its cavity or lumen. In some embodiments, the virus based vector particles are lentivirus. In some embodiments, the lentiviral vector particle is Human Immunodeficiency Virus-1 (HIV-1).

In some aspects, the viral vector particle is limited in the number of polynucleotides that can be packaged. In some embodiments, nucleotides encoding polypeptides to be packaged can be modified such that they retain functional activity with fewer nucleotides in the coding region than that which encodes for the wild-type peptide. Such modifications can include truncations, or other deletions. In some embodiments, more than one polypeptide can be expressed from the same promoter, such that they are fusion polypeptides. In some embodiments, the insert size to be packaged (i.e., viral genome, or portions thereof; or heterologous polynucleotides as described) can be between 500-1000, 1000-2000, 2000-3000, 3000-4000, 4000-5000, 5000-6000, 6000-7000, or 7000-8000 nucleotides in length. In some embodiments, the insert can be over 8000 nucleotides, such as 9000, 10,000, or 11,000 nucleotides in length.

In some embodiments, the viral vector particle, such as retroviral vector particle, comprises one or more of gag polyprotein, polymerase (e.g., pol), integrase (e.g., a functional or non-functional variant), protease, and a fusogen. In some embodiments, the lipid particle further comprises rev. In some embodiments, one or more of the aforesaid proteins are encoded in the retroviral genome (i.e., the insert as described above), and in some embodiments, one or more of the aforesaid proteins are provided in trans, e.g., by a helper cell, helper virus, or helper plasmid. In some embodiments, the lipid particle nucleic acid (e.g., retroviral nucleic acid) comprises one or more of the following nucleic acid sequences: 5′ LTR (e.g., comprising U5 and lacking a functional U3 domain), Psi packaging element (Psi, ‘), Central polypurine tract (cPPT) Promoter operatively linked to the payload gene, payload gene (optionally comprising an intron before the open reading frame), Poly A tail sequence, WPRE, and 3’ LTR (e.g., comprising U5 and lacking a functional U3). In some embodiments, the lipid particle nucleic acid further comprises a retroviral cis-acting RNA packaging element, and a cPPT/CTS element. In some embodiments the lipid particle nucleic acid further comprises one or more insulator element. In some embodiments, the recognition sites are situated between the poly A tail sequence and the WPRE.

In some embodiments, the lipid particle comprises supramolecular complexes formed by viral proteins that self-assemble into capsids. In some embodiments, the lipid particle is a viral particle derived from viral capsids. In some embodiments, the lipid particle is a viral particle derived from viral nucleocapsids. In some embodiments, the lipid particle comprises nucleocapsid-derived that retain the property of packaging nucleic acids.

In some embodiments, the lipid particle packages nucleic acids from host cells carrying one or more viral nucleic acids (e.g. retroviral nucleic acids) during the expression process. In some embodiments, the nucleic acids do not encode any genes involved in virus replication. In particular embodiments, the lipid particle is a virus-based particle, e.g. retrovirus particle such as a lentivirus particle, that is replication defective.

In some cases, the lipid particle is a viral particle that is morphologically indistinguishable from the wild type infectious virus. In some embodiments, the viral particle presents the entire viral proteome as an antigen. In some embodiments, the viral particle presents only a portion of the proteome as an antigen.

In some embodiments, the retroviral nucleic acid comprises one or more of (e.g., all of): a 5′ promoter (e.g., to control expression of the entire packaged RNA), a 5′ LTR (e.g., that includes R (polyadenylation tail signal) and/or U5 which includes a primer activation signal), a primer binding site, a psi packaging signal, a RRE element for nuclear export, a promoter directly upstream of the transgene to control transgene expression, a transgene (or other exogenous agent element), a polypurine tract, and a 3′ LTR (e.g., that includes a mutated U3, a R, and U5). In some embodiments, the retroviral nucleic acid further comprises one or more of a cPPT, a WPRE, and/or an insulator element.

A retrovirus typically replicates by reverse transcription of its genomic RNA into a linear double-stranded DNA copy and subsequently covalently integrates its genomic DNA into a host genome. Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

In some embodiments the retrovirus is a Gammaretrovirus. In some embodiments the retrovirus is an Epsilonretrovirus. In some embodiments the retrovirus is an Alpharetrovirus. In some embodiments the retrovirus is a Betaretrovirus. In some embodiments the retrovirus is a Deltaretrovirus. In some embodiments the retrovirus is a Lentivirus. In some embodiments the retrovirus is a Spumaretrovirus. In some embodiments the retrovirus is an endogenous retrovirus.

Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In some embodiments, HIV based vector backbones (i.e., HIV cis-acting sequence elements) are used.

A viral vector can comprise a nucleic acid molecule (e.g., a transfer plasmid) that includes virus-derived nucleic acid elements that typically facilitate transfer of a nucleic acid molecule (e.g. including nucleic acid encoding an exogenous agent) or integration into the genome of a cell or to a viral particle that mediates nucleic acid transfer. Viral vector particles will typically include various viral components and sometimes also host cell components in addition to nucleic acid(s). A viral vector can comprise a virus or viral particle capable of transferring a nucleic acid into a cell (e.g. nucleic acid encoding an exogenous agent), or to the transferred nucleic acid (e.g., as naked DNA). Viral vectors and transfer plasmids can comprise structural and/or functional genetic elements that are primarily derived from a virus. A retroviral vector can comprise a viral vector or plasmid containing structural and functional genetic elements, or portions thereof, that are primarily derived from a retrovirus. A lentiviral vector can comprise a viral vector or plasmid containing structural and functional genetic elements, or portions thereof, including LTRs that are primarily derived from a lentivirus.

In embodiments, a lentiviral vector (e.g., lentiviral expression vector) may comprise a lentiviral transfer plasmid (e.g., as naked DNA) or an infectious lentiviral particle. With respect to elements such as cloning sites, promoters, regulatory elements, heterologous nucleic acids, etc., it is to be understood that the sequences of these elements can be present in RNA form in lentiviral particles and can be present in DNA form in DNA plasmids.

In some vectors described herein, at least part of one or more protein coding regions that contribute to or are essential for replication may be absent compared to the corresponding wild-type virus. This makes the viral vector replication-defective. In some embodiments, the vector is capable of transducing a target non-dividing host cell and/or integrating its genome into a host genome.

The structure of a wild-type retrovirus genome often comprises a 5′ long terminal repeat (LTR) and a 3′ LTR, between or within which are located a packaging signal to enable the genome to be packaged, a primer binding site, integration sites to enable integration into a host cell genome and gag, pol and env genes encoding the packaging components which promote the assembly of viral particles. More complex retroviruses have additional features, such as rev and RRE sequences in HIV, which enable the efficient export of RNA transcripts of the integrated provirus from the nucleus to the cytoplasm of an infected target cell. In the provirus, the viral genes are flanked at both ends by regions called long terminal repeats (LTRs). The LTRs are involved in proviral integration and transcription. LTRs also serve as enhancer-promoter sequences and can control the expression of the viral genes. Encapsidation of the retroviral RNAs occurs by virtue of a psi sequence located at the 5′ end of the viral genome.

The LTRs themselves are typically similar (e.g., identical) sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

For the viral genome, the site of transcription initiation is typically at the boundary between U3 and R in one LTR and the site of poly (A) addition (termination) is at the boundary between R and U5 in the other LTR. U3 contains most of the transcriptional control elements of the provirus, which include the promoter and multiple enhancer sequences responsive to cellular and in some cases, viral transcriptional activator proteins. Some retroviruses comprise any one or more of the following genes that code for proteins that are involved in the regulation of gene expression: tot, rev, tax and rex. With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome. The env gene encodes the surface (SU) glycoprotein and the transmembrane (TM) protein of the virion, which form a complex that interacts specifically with cellular receptor proteins. This interaction promotes infection, e.g., by fusion of the viral membrane with the cell membrane.

In a replication-defective retroviral vector genome gag, pol and env may be absent or not functional. The R regions at both ends of the RNA are typically repeated sequences. U5 and U3 represent unique sequences at the 5′ and 3′ ends of the RNA genome respectively.

Retroviruses may also contain additional genes which code for proteins other than gag, pol and env. Examples of additional genes include (in HIV), one or more of vif, vpr, vpx, vpu, tat, rev and nef. EIAV has (amongst others) the additional gene S2. Proteins encoded by additional genes serve various functions, some of which may be duplicative of a function provided by a cellular protein. In EIAV, for example, tat acts as a transcriptional activator of the viral LTR (Derse and Newbold 1993 Virology 194:530-6; Maury et al. 1994 Virology 200:632-42). It binds to a stable, stem-loop RNA secondary structure referred to as TAR. Rev regulates and co-ordinates the expression of viral genes through rev-response elements (RRE) (Martarano et al. 1994 J. Virol. 68:3102-11). The mechanisms of action of these two proteins are thought to be broadly similar to the analogous mechanisms in the primate viruses. In addition, an EIAV protein, Ttm, has been identified that is encoded by the first exon of tat spliced to the env coding sequence at the start of the transmembrane protein.

In addition to protease, reverse transcriptase and integrase, non-primate lentiviruses contain a fourth pol gene product which codes for a dUTPase. This may play a role in the ability of these lentiviruses to infect certain non-dividing or slowly dividing cell types.

In embodiments, a recombinant lentiviral vector (RLV) is a vector with sufficient retroviral genetic information to allow packaging of an RNA genome, in the presence of packaging components, into a viral particle capable of infecting a target cell. Infection of the target cell can comprise reverse transcription and integration into the target cell genome. The RLV typically carries non-viral coding sequences which are to be delivered by the vector to the target cell, such as nucleic acid encoding an exogenous agent as described herein. In embodiments, an RLV is incapable of independent replication to produce infectious retroviral particles within the target cell. Usually the RLV lacks a functional gag-pol and/or env gene and/or other genes involved in replication. The vector may be configured as a split-intron vector, e.g., as described in PCT patent application WO 99/15683, which is herein incorporated by reference in its entirety.

In some embodiments, the lentiviral vector comprises a minimal viral genome, e.g., the viral vector has been manipulated so as to remove the non-essential elements and to retain the essential elements in order to provide the required functionality to infect, transduce and deliver a nucleotide sequence of interest to a target host cell, e.g., as described in WO 98/17815, which is herein incorporated by reference in its entirety.

A minimal lentiviral genome may comprise, e.g., (5′) R-U5-one or more first nucleotide sequences-U3-R (3′). However, the plasmid vector used to produce the lentiviral genome within a source cell can also include transcriptional regulatory control sequences operably linked to the lentiviral genome to direct transcription of the genome in a source cell. These regulatory sequences may comprise the natural sequences associated with the transcribed retroviral sequence, e.g., the 5′ U3 region, or they may comprise a heterologous promoter such as another viral promoter, for example the CMV promoter. Some lentiviral genomes comprise additional sequences to promote efficient virus production. For example, in the case of HIV, rev and RRE sequences may be included. Alternatively or in combination, codon optimization may be used, e.g., the gene encoding the exogenous agent may be codon optimized, e.g., as described in WO 01/79518, which is herein incorporated by reference in its entirety. Alternative sequences which perform a similar or the same function as the rev/RRE system may also be used. For example, a functional analogue of the rev/RRE system is found in the Mason Pfizer monkey virus. This is known as CTE and comprises an RRE-type sequence in the genome which is believed to interact with a factor in the infected cell. The cellular factor can be thought of as a rev analogue. Thus, CTE may be used as an alternative to the rev/RRE system. In addition, the Rex protein of HTLV-I can functionally replace the Rev protein of HIV-I. Rev and Rex have similar effects to IRE-BP.

In some embodiments, a retroviral nucleic acid (e.g., a lentiviral nucleic acid, e.g., a primate or non-primate lentiviral nucleic acid) (1) comprises a deleted gag gene wherein the deletion in gag removes one or more nucleotides downstream of about nucleotide 350 or 354 of the gag coding sequence; (2) has one or more accessory genes absent from the retroviral nucleic acid; (3) lacks the tat gene but includes the leader sequence between the end of the 5′ LTR and the ATG of gag; and (4) combinations of (1), (2) and (3). In an embodiment the lentiviral vector comprises all of features (1) and (2) and (3). This strategy is described in more detail in WO 99/32646, which is herein incorporated by reference in its entirety.

In some embodiments, a primate lentivirus minimal system requires none of the HIV/SIV additional genes vif, vpr, vpx, vpu, tat, rev and nef for either vector production or for transduction of dividing and non-dividing cells. In some embodiments, an EIAV minimal vector system does not require S2 for either vector production or for transduction of dividing and non dividing cells.

The deletion of additional genes may permit vectors to be produced without the genes associated with disease in lentiviral (e.g. HIV) infections. In particular, tat is associated with disease. Secondly, the deletion of additional genes permits the vector to package more heterologous DNA. Thirdly, genes whose function is unknown, such as S2, may be omitted, thus reducing the risk of causing undesired effects. Examples of minimal lentiviral vectors are disclosed in WO 99/32646 and in WO 98/17815.

In some embodiments, the retroviral nucleic acid is devoid of at least tat and S2 (if it is an EIAV vector system), and possibly also vif, vpr, vpx, vpu and nef. In some embodiments, the retroviral nucleic acid is also devoid of rev, RRE, or both.

In some embodiments the retroviral nucleic acid comprises vpx. The Vpx polypeptide binds to and induces the degradation of the SAMHD1 restriction factor, which degrades free dNTPs in the cytoplasm. Thus, the concentration of free dNTPs in the cytoplasm increases as Vpx degrades SAMHD1 and reverse transcription activity is increased, thus facilitating reverse transcription of the retroviral genome and integration into the target cell genome.

Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available. An additional description of codon optimization is found, e.g., in WO 99/41397, which is herein incorporated by reference in its entirety.

Many viruses, including HIV and other lentiviruses, use a large number of rare codons and by changing these to correspond to commonly used mammalian codons, increased expression of the packaging components in mammalian producer cells can be achieved.

In some embodiments, codon optimization has a number of other advantages. In some embodiments, by virtue of alterations in their sequences, the nucleotide sequences encoding the packaging components may have RNA instability sequences (INS) reduced or eliminated from them. At the same time, the amino acid sequence coding sequence for the packaging components is retained so that the viral components encoded by the sequences remain the same, or at least sufficiently similar that the function of the packaging components is not compromised. In some embodiments, codon optimization also overcomes the Rev/RRE requirement for export, rendering optimized sequences Rev independent. In some embodiments, codon optimization also reduces homologous recombination between different constructs within the vector system (for example between the regions of overlap in the gag-pol and env open reading frames). In some embodiments, codon optimization leads to an increase in viral titer and/or improved safety.

In some embodiments, only codons relating to INS are codon optimized. In other embodiments, the sequences are codon optimized in their entirety, with the exception of the sequence encompassing the frameshift site of gag-pol.

The gag-pol gene comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is preferably not codon optimized. In some embodiments, retaining this fragment will enable more efficient expression of the gag-pol proteins. For EIAV, the beginning of the overlap is at nt 1262 (where nucleotide 1 is the A of the gag ATG). The end of the overlap is at nt 1461. In order to ensure that the frameshift site and the gag-pol overlap are preserved, the wild type sequence may be retained from nt 1156 to 1465.

In some embodiments, derivations from optimal codon usage may be made, for example, in order to accommodate convenient restriction sites, and conservative amino acid changes may be introduced into the gag-pol proteins.

In some embodiments, codon optimization is based on codons with poor codon usage in mammalian systems. The third and sometimes the second and third base may be changed.

In some embodiments, due to the degenerate nature of the genetic code, it will be appreciated that numerous gag-pol sequences can be achieved by a skilled worker. Also, there are many retroviral variants described which can be used as a starting point for generating a codon optimized gag-pol sequence. Lentiviral genomes can be quite variable. For example there are many quasi-species of HIV-I which are still functional. This is also the case for EIAV. These variants may be used to enhance particular parts of the transduction process. Examples of HIV-I variants may be found in the HIV databases maintained by Los Alamos National Laboratory. Details of EIAV clones may be found at the NCBI database maintained by the National Institutes of Health.

It is within the level of a skilled artisan to empirically determine appropriate codon optimization of viral sequences. The strategy for codon optimized sequences, including gag-pol sequences, can be used in relation to any retrovirus, e.g., EIAV, FIV, BIV, CAEV, VMR, SIV, HIV—I and HIV-2. In addition this method can be used to increase expression of genes from HTLV-I, HTLV-2, HFV, HSRV and human endogenous retroviruses (HERV), MLV and other retroviruses.

In embodiments, the retroviral vector comprises a packaging signal that comprises from 255 to 360 nucleotides of gag in vectors that still retain env sequences, or about 40 nucleotides of gag in a particular combination of splice donor mutation, gag and env deletions. In some embodiments, the retroviral vector includes a gag sequence which comprises one or more deletions, e.g., the gag sequence comprises about 360 nucleotides derivable from the N-terminus.

In some embodiments, the retroviral vector, helper cell, helper virus, or helper plasmid may comprise retroviral structural and accessory proteins, for example gag, pol, env, tat, rev, vif, vpr, vpu, vpx, or nef proteins or other retroviral proteins. In some embodiments the retroviral proteins are derived from the same retrovirus. In some embodiments the retroviral proteins are derived from more than one retrovirus, e.g. 2, 3, 4, or more retroviruses.

In some embodiments, the gag and pol coding sequences are generally organized as the Gag-Pol Precursor in native lentivirus. The gag sequence codes for a 55-kD Gag precursor protein, also called p55. The p55 is cleaved by the virally encoded protease (a product of the pol gene) during the process of maturation into four smaller proteins designated MA (matrix [p17]), CA (capsid [p24]), NC (nucleocapsid [p9]), and p6. The pol precursor protein is cleaved away from Gag by a virally encoded protease, and further digested to separate the protease (p10), RT (p50), RNase H (p15), and integrase (p31) activities.

In some embodiments, the lentiviral vector is integration-deficient. In some embodiments, the pol is integrase deficient, such as by encoding due to mutations in the integrase gene. For example, the pol coding sequence can contain an inactivating mutation in the integrase, such as by mutation of one or more of amino acids involved in catalytic activity, i.e. mutation of one or more of aspartic 64, aspartic acid 116 and/or glutamic acid 152. In some embodiments, the integrase mutation is a D64V mutation. In some embodiments, the mutation in the integrase allows for packaging of viral RNA into a lentivirus. In some embodiments, the mutation in the integrase allows for packaging of viral proteins into a lentivirus. In some embodiments, the mutation in the integrase reduces the possibility of insertional mutagenesis. In some embodiments, the mutation in the integrase decreases the possibility of generating replication-competent recombinants (RCRs) (Wanisch et al. 2009. Mol Ther. 1798): 1316-1332). In some embodiments, native Gag-Pol sequences can be utilized in a helper vector (e.g., helper plasmid or helper virus), or modifications can be made. These modifications include, chimeric Gag-Pol, where the Gag and Pol sequences are obtained from different viruses (e.g., different species, subspecies, strains, clades, etc.), and/or where the sequences have been modified to improve transcription and/or translation, and/or reduce recombination.

In some embodiments, the retroviral nucleic acid includes a polynucleotide encoding a 150-250 (e.g., 168) nucleotide portion of a gag protein that (i) includes a mutated INS1 inhibitory sequence that reduces restriction of nuclear export of RNA relative to wild-type INS1, (ii) contains two nucleotide insertion that results in frame shift and premature termination, and/or (iii) does not include INS2, INS3, and INS4 inhibitory sequences of gag.

In some embodiments, a vector described herein is a hybrid vector that comprises both retroviral (e.g., lentiviral) sequences and non-lentiviral viral sequences. In some embodiments, a hybrid vector comprises retroviral e.g., lentiviral, sequences for reverse transcription, replication, integration and/or packaging.

According to certain specific embodiments, most or all of the viral vector backbone sequences are derived from a lentivirus, e.g., HIV-1. However, it is to be understood that many different sources of retroviral and/or lentiviral sequences can be used, or combined and numerous substitutions and alterations in certain of the lentiviral sequences may be accommodated without impairing the ability of a transfer vector to perform the functions described herein. A variety of lentiviral vectors are described in Naldini et ah, (1996a, 1996b, and 1998); Zufferey et al., (1997); Dull et al., 1998, U.S. Pat. Nos. 6,013,516; and 5,994,136, many of which may be adapted to produce a retroviral nucleic acid.

At each end of the provirus, long terminal repeats (LTRs) are typically found. An LTR typically comprises a domain located at the ends of retroviral nucleic acid which, in their natural sequence context, are direct repeats and contain U3, R and U5 regions. LTRs generally promote the expression of retroviral genes (e.g., promotion, initiation and polyadenylation of gene transcripts) and viral replication. The LTR can comprise numerous regulatory signals including transcriptional control elements, polyadenylation signals and sequences for replication and integration of the viral genome. The viral LTR is typically divided into three regions called U3, R and U5. The U3 region typically contains the enhancer and promoter elements. The U5 region is typically the sequence between the primer binding site and the R region and can contain the polyadenylation sequence. The R (repeat) region can be flanked by the U3 and U5 regions. The LTR is typically composed of U3, R and U5 regions and can appear at both the 5′ and 3′ ends of the viral genome. In some embodiments, adjacent to the 5′ LTR are sequences for reverse transcription of the genome (the tRNA primer binding site) and for efficient packaging of viral RNA into particles (the Psi site).

In some embodiments, a packaging signal can comprise a sequence located within the retroviral genome which mediate insertion of the viral RNA into the viral capsid or particle, see e.g., Clever et al., 1995. J. of Virology, Vol. 69, No. 4; pp. 2101-2109. Several retroviral vectors use a minimal packaging signal (a psi [Ψ] sequence) for encapsidation of the viral genome.

In various embodiments, retroviral nucleic acids comprise modified 5′ LTR and/or 3′ LTRs. Either or both of the LTR may comprise one or more modifications including, but not limited to, one or more deletions, insertions, or substitutions. Modifications of the 3′ LTR are often made to improve the safety of lentiviral or retroviral systems by rendering viruses replication-defective, e.g., virus that is not capable of complete, effective replication such that infective virions are not produced (e.g., replication-defective lentiviral progeny).

In some embodiments, a vector is a self-inactivating (SIN) vector, e.g., replication-defective vector, e.g., retroviral or lentiviral vector, in which the right (3′) LTR enhancer-promoter region, known as the U3 region, has been modified (e.g., by deletion or substitution) to prevent viral transcription beyond the first round of viral replication. In some aspects, provided herein is a replication incompetent (also referred to herein as replication defective) vector particle, that cannot participate in replication in the absence of the packaging cell (i.e., viral vector particles are not produced from the transduced cell). In some aspects, this is because the right (3′) LTR U3 region can be used as a template for the left (5′) LTR U3 region during viral replication and, thus, absence of the U3 enhancer-promoter inhibits viral replication. In embodiments, the 3′ LTR is modified such that the U5 region is removed, altered, or replaced, for example, with an exogenous poly (A) sequence The 3′ LTR, the 5′ LTR, or both 3′ and 5′ LTRs, may be modified LTRs. Other modifications to the viral vector, i.e., retroviral or lentiviral vector, to render said vector replication incompetent are known in the art.

In some embodiments, the U3 region of the 5′ LTR is replaced with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. In some embodiments, promoters are able to drive high levels of transcription in a Tat-independent manner. In certain embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include, but are not limited to, one or more chemical compounds or the physiological conditions such as temperature or pH, in which the host cells are cultured.

In some embodiments, viral vectors comprise a TAR (trans-activation response) element, e.g., located in the R region of lentiviral (e.g., HIV) LTRs. This element interacts with the lentiviral trans-activator (tat) genetic element to enhance viral replication. However, this element is not required, e.g., in embodiments wherein the U3 region of the 5′ LTR is replaced by a heterologous promoter.

The R region, e.g., the region within retroviral LTRs beginning at the start of the capping group (i.e., the start of transcription) and ending immediately prior to the start of the poly A tract can be flanked by the U3 and U5 regions. The R region plays a role during reverse transcription in the transfer of nascent DNA from one end of the genome to the other.

The retroviral nucleic acid can also comprise a FLAP element, e.g., a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et ah, 2000, Cell, 101:173, which are herein incorporated by reference in their entireties. During HIV-1 reverse transcription, central initiation of the plus-strand DNA at the central polypurine tract (cPPT) and central termination at the central termination sequence (CTS) can lead to the formation of a three-stranded DNA structure: the HIV-1 central DNA flap. In some embodiments, the retroviral or lentiviral vector backbones comprise one or more FLAP elements upstream or downstream of the gene encoding the exogenous agent. For example, in some embodiments a transfer plasmid includes a FLAP element, e.g., a FLAP element derived or isolated from HIV-L

In embodiments, a retroviral or lentiviral nucleic acid comprises one or more export elements, e.g., a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65:1053; and Cullen et al., 1991. Cell 58:423), and the hepatitis B virus post-transcriptional regulatory element (HPRE), which are herein incorporated by reference in their entireties. Generally, the RNA export element is placed within the 3′ UTR of a gene, and can be inserted as one or multiple copies.

In some embodiments, expression of heterologous sequences (e.g. nucleic acid encoding an exogenous agent) in viral vectors is increased by incorporating one or more of, e.g., all of, posttranscriptional regulatory elements, polyadenylation sites, and transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766), each of which is herein incorporated by reference in its entirety. In some embodiments, a retroviral nucleic acid described herein comprises a posttranscriptional regulatory element such as a WPRE or HPRE

In some embodiments, a retroviral nucleic acid described herein lacks or does not comprise a posttranscriptional regulatory element such as a WPRE or HPRE.

Elements directing the termination and polyadenylation of the heterologous nucleic acid transcripts may be included, e.g., to increases expression of the exogenous agent. Transcription termination signals may be found downstream of the polyadenylation signal. In some embodiments, vectors comprise a polyadenylation sequence 3′ of a polynucleotide encoding the exogenous agent. A polyA site may comprise a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Illustrative examples of polyA signals that can be used in a retroviral nucleic acid, include AATAAA, ATT AAA, AGTAAA, a bovine growth hormone polyA sequence (BGHpA), a rabbit b-globin polyA sequence (rPgpA), or another suitable heterologous or endogenous polyA sequence.

In some embodiments, a retroviral or lentiviral vector further comprises one or more insulator elements, e.g., an insulator element described herein.

In various embodiments, the vectors comprise a promoter operably linked to a polynucleotide encoding an exogenous agent. The vectors may have one or more LTRs, wherein either LTR comprises one or more modifications, such as one or more nucleotide substitutions, additions, or deletions. The vectors may further comprise one of more accessory elements to increase transduction efficiency (e.g., a cPPT/FLAP), viral packaging (e.g., a Psi (Y′) packaging signal, RRE), and/or other elements that increase exogenous gene expression (e.g., poly (A) sequences), and may optionally comprise a WPRE or HPRE.

In some embodiments, a lentiviral nucleic acid comprises one or more of, e.g., all of, e.g., from 5′ to 3′, a promoter (e.g., CMV), an R sequence (e.g., comprising TAR), a U5 sequence (e.g., for integration), a PBS sequence (e.g., for reverse transcription), a DIS sequence (e.g., for genome dimerization), a psi packaging signal, a partial gag sequence, an RRE sequence (e.g., for nuclear export), a cPPT sequence (e.g., for nuclear import), a promoter to drive expression of the exogenous agent, a gene encoding the exogenous agent, a WPRE sequence (e.g., for efficient transgene expression), a PPT sequence (e.g., for reverse transcription), an R sequence (e.g., for polyadenylation and termination), and a U5 signal (e.g., for integration).

### 2. Virus-Like Particles

In some embodiments, the viral-based particles are viral-like lipid particles (VLPs) that are derived from virus. In some embodiments, the viral envelope may comprise a fusogen, e.g., a fusogen that is endogenous to the virus or a pseudotyped fusogen, e.g., a retargeted attachment protein such as a G or F protein described in Section II. The VLPs include those derived from retroviruses or lentiviruses. While VLPs mimic native virion structure, they lack the viral genomic information necessary for independent replication within a host cell. Therefore, in some aspects, VLPs are non-infectious. In particular embodiments, a VLP does not contain a viral genome. In some embodiments, the VLP's bilayer of amphipathic lipids is or comprises the viral envelope. In some embodiments, the lipid particle's bilayer of amphipathic lipids is or comprises lipids derived from a cell. In some embodiments, a VLP contains at least one type of structural protein from a virus. In most cases this protein will form a proteinaceous capsid. In some cases the capsid will also be enveloped in a lipid bilayer originating from the cell from which the assembled VLP has been released (e.g. VLPs comprising a human immunodeficiency virus structural protein such as GAG). In some embodiments, the VLP further comprises a targeting moiety as an envelope protein within the lipid bilayer.

In some embodiments, the vector vehicle particle comprises supramolecular complexes formed by viral proteins that self-assemble into capsids. In some embodiments, the vector vehicle particle is a virus-like particle derived from viral capsid proteins. In some embodiments, the vector vehicle particle is a virus-like particle derived from viral nucleocapsid proteins. In some embodiments, the vector vehicle particle comprises nucleocapsid-derived proteins that retain the property of packaging nucleic acids. In some embodiments, the viral-based particles, such as virus-like particles comprises only viral structural glycoproteins among proteins from the viral genome. In some embodiments, the vector vehicle particle does not contain a viral genome.

In some embodiments, the vector vehicle particle packages nucleic acids from host cells during the expression process, such as a nucleic acid encoding an exogenous agent. In some embodiments, the nucleic acids do not encode any genes involved in virus replication. In particular embodiments, the vector vehicle particle is a virus-like particle, e.g. retrovirus-like particle such as a lentivirus-like particle, that is replication defective.

In some embodiments, the vector vehicle particle is a virus-like particle which comprises a sequence that is devoid of or lacking viral RNA may be the result of removing or eliminating the viral RNA from the sequence. In some embodiments, this may be achieved by using an endogenous packaging signal binding site on gag. In some embodiments, the endogenous packaging signal binding site is on pol. In some embodiments, the RNA which is to be delivered will contain a cognate packaging signal. In some embodiments, a heterologous binding domain (which is heterologous to gag) located on the RNA to be delivered, and a cognate binding site located on gag or pol, can be used to ensure packaging of the RNA to be delivered. In some embodiments, the heterologous sequence can be non-viral or it can be viral, in which case it may be derived from a different virus. In some embodiments, the vector particles can be used to deliver therapeutic RNA, in which case functional integrase and/or reverse transcriptase is not required. In some embodiments, the vector particles can also be used to deliver a therapeutic gene of interest, in which case pol is typically included.

In some embodiments, the VLP comprises supramolecular complexes formed by viral proteins that self-assemble into capsids. In some embodiments, the VLP is derived from viral capsids. In some embodiments, the VLP is derived from viral nucleocapsids. In some embodiments, the VLP is nucleocapsid-derived and retains the property of packaging nucleic acids. In some embodiments, the VLP includes only viral structural glycoproteins. In some embodiments, the VLP does not contain a viral genome.

### 3. Methods of Generating Viral-Based Particles

Large scale viral particle production is often useful to achieve a desired viral titer. Viral particles can be produced by transfecting a transfer vector into a packaging cell line that comprises viral structural and/or accessory genes, e.g., gag, pol, env, tat, rev, vif, vpr, vpu, vpx, or nef genes or other retroviral genes.

In some embodiments, viral vector particles may be produced in multiple cell culture systems including bacteria, mammalian cell lines, insect cell lines, yeast and plant cells. Methods of producing such viral-based particles can also be found in U.S. Pat. No. 10,316,295, which is incorporated herein by reference. Exemplary methods for producing viral vector particles are described.

In some embodiments, elements for the production of a viral vector, i.e., a recombinant viral vector such as a replication incompetent lentiviral vector, are included in a packaging cell line or are present on a packaging vector. In some embodiments, viral vectors can include packaging elements, rev, gag, and pol, delivered to the packaging cells line via one or more packaging vectors.

In embodiments, the packaging vector is an expression vector or viral vector that lacks a packaging signal and comprises a polynucleotide encoding one, two, three, four or more viral structural and/or accessory genes. Typically, the packaging vectors are included in a packaging cell, and are introduced into the cell via transfection, transduction or infection. A retroviral, e.g., lentiviral, transfer vector can be introduced into a packaging cell line, via transfection, transduction or infection, to generate a source cell or cell line. The packaging vectors can be introduced into human cells or cell lines by standard methods including, e.g., calcium phosphate transfection, lipofection or electroporation. In some embodiments, the packaging vectors are introduced into the cells together with a dominant selectable marker, such as neomycin, hygromycin, puromycin, blastocidin, zeocin, thymidine kinase, DHFR, Gln synthetase or ADA, followed by selection in the presence of the appropriate drug and isolation of clones. A selectable marker gene can be linked physically to genes encoding by the packaging vector, e.g., by IRES or self-cleaving viral peptides. In some embodiments, the packaging vector is a packaging plasmid.

Producer cell lines (also called packaging cell lines) include cell lines that do not contain a packaging signal, but do stably or transiently express viral structural proteins and replication enzymes (e.g., gag, pol and env) which can package viral particles. Any suitable cell line can be employed, e.g., mammalian cells, e.g., human cells. Suitable cell lines which can be used include, for example, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211 A cells. In embodiments, the packaging cells are 293 cells, 293T cells, or A549 cells.

In some embodiments, a producer cell (i.e., a source cell line) includes a cell line which is capable of producing recombinant retroviral particles, comprising a packaging cell line and a transfer vector construct comprising a packaging signal. Methods of preparing viral stock solutions are illustrated by, e.g., Y. Soneoka et al. (1995) Nucl. Acids Res. 23:628-633, and N. R. Landau et al. (1992) J. Virol. 66:5110-5113, which are incorporated herein by reference. Infectious virus particles may be collected from the packaging cells, e.g., by cell lysis, or collection of the supernatant of the cell culture. Optionally, the collected virus particles may be enriched or purified.

In some embodiments, the source cell comprises one or more plasmids coding for viral structural proteins and replication enzymes (e.g., gag, pol and env) which can package viral particles (i.e., a packaging plasmid). In some embodiments, the sequences coding for at least two of the gag, pol, and env precursors are on the same plasmid. In some embodiments, the sequences coding for the gag, pol, and env precursors are on different plasmids. In some embodiments, the sequences coding for the gag, pol, and env precursors have the same expression signal, e.g., promoter. In some embodiments, the sequences coding for the gag, pol, and env precursors have a different expression signal, e.g., different promoters. In some embodiments, expression of the gag, pol, and env precursors is inducible. In some embodiments, the plasmids coding for viral structural proteins and replication enzymes are transfected at the same time or at different times. In some embodiments, the plasmids coding for viral structural proteins and replication enzymes are transfected at the same time or at a different time from the packaging vector.

In some embodiments, the source cell line comprises one or more stably integrated viral structural genes. In some embodiments expression of the stably integrated viral structural genes is inducible.

In some embodiments, expression of the viral structural genes is regulated at the transcriptional level. In some embodiments, expression of the viral structural genes is regulated at the translational level. In some embodiments, expression of the viral structural genes is regulated at the post-translational level.

In some embodiments, expression of the viral structural genes is regulated by a tetracycline (Tet)-dependent system, in which a Tet-regulated transcriptional repressor (Tet-R) binds to DNA sequences included in a promoter and represses transcription by steric hindrance (Yao et al, 1998; Jones et al, 2005). Upon addition of doxycycline (dox), Tet-R is released, allowing transcription. Multiple other suitable transcriptional regulatory promoters, transcription factors, and small molecule inducers are suitable to regulate transcription of viral structural genes.

In some embodiments, the third-generation lentivirus components, human immunodeficiency virus type 1 (HIV) Rev, Gag/Pol, and an envelope under the control of Tet-regulated promoters and coupled with antibiotic resistance cassettes are separately integrated into the source cell genome. In some embodiments the source cell only has one copy of each of Rev, Gag/Pol, and an envelope protein integrated into the genome.

In some embodiments a nucleic acid encoding the exogenous agent (e.g., a retroviral nucleic acid encoding the exogenous agent) is also integrated into the source cell genome. In some embodiments a nucleic acid encoding the exogenous agent is maintained episomally. In some embodiments a nucleic acid encoding the exogenous agent is transfected into the source cell that has stably integrated Rev, Gag/Pol, and an envelope protein in the genome. See, e.g., Milani et al. EMBO Molecular Medicine, 2017, which is herein incorporated by reference in its entirety.

In some embodiments, a retroviral nucleic acid described herein is unable to undergo reverse transcription. Such a nucleic acid, in embodiments, is able to transiently express an exogenous agent. The retrovirus or VLP, may comprise a disabled reverse transcriptase protein, or may not comprise a reverse transcriptase protein. In embodiments, the retroviral nucleic acid comprises a disabled primer binding site (PBS) and/or att site. In embodiments, one or more viral accessory genes, including rev, tat, vif, nef, vpr, vpu, vpx and S2 or functional equivalents thereof, are disabled or absent from the retroviral nucleic acid. In embodiments, one or more accessory genes selected from S2, rev and tat are disabled or absent from the retroviral nucleic acid.

Typically, modern retroviral vector systems include viral genomes bearing cis-acting vector sequences for transcription, reverse-transcription, integration, translation and packaging of viral RNA into the viral particles, and (2) producer cells lines which express the trans-acting retroviral gene sequences (e.g., gag, pol and env) needed for production of virus particles. By separating the cis- and trans-acting vector sequences completely, the virus is unable to maintain replication for more than one cycle of infection. Generation of live virus can be avoided by a number of strategies, e.g., by minimizing the overlap between the cis- and trans-acting sequences to avoid recombination.

A virus-like particle (VLP) which comprises a sequence that is devoid of or lacking viral RNA as described in Section III.A.2 may be the result of removing or eliminating the viral RNA from the sequence. Similar to the viral vector particles disclosed in Section III.A.1, VLPs contain a viral outer envelope made from the host cell (i.e., producer cell or source cell) lipid-bi layer as well as at least one viral structural protein. In some embodiments, a viral structural protein refers to any viral protein or fragment thereof which contributes to the structure of the viral core or capsid.

Generally, for viral vector particles, expression of the gag precursor protein alone mediates vector assembly and release. In some aspects, gag proteins or fragments thereof have been demonstrated to assemble into structures analogous to viral cores. In one embodiment this may be achieved by using an endogenous packaging signal binding site on gag. Alternatively, the endogenous packaging signal binding site is on pol. In this embodiment, the RNA which is to be delivered will contain a cognate packaging signal. In another embodiment, a heterologous binding domain (which is heterologous to gag) located on the RNA to be delivered, and a cognate binding site located on gag or pol, can be used to ensure packaging of the RNA to be delivered. The heterologous sequence can be non-viral or it can be viral, in which case it may be derived from a different virus. The VLP can be used to deliver therapeutic RNA, in which case functional integrase and/or reverse transcriptase is not required. These VLPs can also be used to deliver a therapeutic gene of interest, in which case pol is typically included.

In an embodiment, gag-pol are altered, and the packaging signal is replaced with a corresponding packaging signal. In this embodiment, the particle can package the RNA with the new packaging signal. The advantage of this approach is that it is possible to package an RNA sequence which is devoid of viral sequence for example, RNAi.

An alternative approach is to rely on over-expression of the RNA to be packaged. In one embodiment the RNA to be packaged is over-expressed in the absence of any RNA containing a packaging signal. This may result in a significant level of therapeutic RNA being packaged, and that this amount is sufficient to transduce a cell and have a biological effect.

In some embodiments, a polynucleotide comprises a nucleotide sequence encoding a viral gag protein or retroviral gag and pol proteins, wherein the gag protein or pol protein comprises a heterologous RNA binding domain capable of recognizing a corresponding sequence in an RNA sequence to facilitate packaging of the RNA sequence into a viral vector particle. In some embodiments, the heterologous RNA binding domain comprises an RNA binding domain derived from a bacteriophage coat protein, a Rev protein, a protein of the U 1 small nuclear ribonucleoprotein particle, a Nova protein, a TF111 A protein, a TIS 11 protein, a trp RNA-binding attenuation protein (TRAP) or a pseudouridine synthase.

In some embodiments, the assembly of a viral based vector particle (i.e., a VLP) is initiated by binding of the core protein to a unique encapsidation sequence within the viral genome (e.g. UTR with stem-loop structure). In some embodiments, the interaction of the core with the encapsidation sequence facilitates oligomerization.

In some embodiments, the source cell for VLP production comprises one or more plasmids coding for viral structural proteins (e.g., gag, pol) which can package viral particles (i.e., a packaging plasmid). In some embodiments, the sequences coding for at least two of the gag and pol precursors are on the same plasmid. In some embodiments, the sequences coding for the gag and pol precursors are on different plasmids. In some embodiments, the sequences coding for the gag and pol precursors have the same expression signal, e.g., promoter. In some embodiments, the sequences coding for the gag and pol precursors have a different expression signal, e.g., different promoters. In some embodiments, expression of the gag and pol precursors is inducible.

In some embodiments, formation of VLPs or any viral-based particle, such as described above in Section III, can be detected by any suitable technique known in the art. Examples of such techniques include, e.g., electron microscopy, dynamic light scattering, selective chromatographic separation and/or density gradient centrifugation.

### B. Exogenous Agent

In some embodiments, the lipid particle as described herein or pharmaceutical composition comprising same described contains an exogenous agent. In some embodiments, the lipid particle or pharmaceutical composition comprising same described herein contains a nucleic acid that encodes an exogenous agent. In some embodiments, the lipid particle contains the exogenous agent. In some embodiments, the lipid particle contains a nucleic acid that encodes an exogenous agent. Reference to the coding sequence of the nucleic acid encoding the exogenous agent also is referred to herein as a payload gene. In some embodiments, the exogenous agent or the nucleic acid encoding the exogenous agent are present in the lumen of the lipid particle.

In some embodiments, the exogenous agent is a protein or a nucleic acid (e.g., a DNA, a chromosome (e.g. a human artificial chromosome), an RNA, e.g., an mRNA or miRNA). In some embodiments, the exogenous agent comprises or encodes a membrane protein. In some embodiments, the exogenous agent comprises or encodes a therapeutic agent. In some embodiments, the therapeutic agent is chosen from one or more of a protein, e.g., an enzyme, a transmembrane protein, a receptor, or an antibody; a nucleic acid, e.g., DNA, a chromosome (e.g. a human artificial chromosome), RNA, mRNA, siRNA, or miRNA; or a small molecule.

In some embodiments, the lipid particle or pharmaceutical composition delivers to a target cell at least 10%, 20%, 30%, 40%, 50%, 60%, 70%, 80%, 90%, 95%, 96%, 97%, 98%, or 99% of the exogenous agent (e.g., an exogenous agent comprising or encoding a therapeutic agent) comprised by the lipid particle. In some embodiments, the lipid particle, e.g., fusosome, that contacts, e.g., fuses, with the target cell(s) delivers to the target cell an average of at least 10%, 20%, 30%, 40%, 50%, 60%, 70%, 80%, 90%, 95%, 96%, 97%, 98%, or 99% of the exogenous agent (e.g., an exogenous agent comprising or encoding a therapeutic agent) comprised by the lipid particles, e.g., fusosomes, that contact, e.g., fuse, with the target cell(s). In some embodiments, the lipid particle composition delivers to a target tissue at least 10%, 20%, 30%, 40%, 50%, 60%, 70%, 80%, 90%, 95%, 96%, 97%, 98%, or 99% of the exogenous agent (e.g., an exogenous agent comprising or encoding a therapeutic agent) comprised by the lipid particle compositions.

In some embodiments, the exogenous agent is not expressed naturally in the cell from which the lipid particle is derived. In some embodiments, the exogenous agent is expressed naturally in the cell from which the lipid particle is derived. In some embodiments, the exogenous agent is loaded into the lipid particle via expression in the cell from which the lipid particle is derived (e.g. expression from DNA or mRNA introduced via transfection, transduction, or electroporation). In some embodiments, the exogenous is expressed from DNA integrated into the genome or maintained episomally. In some embodiments, expression of the exogenous agent is constitutive. In some embodiments, expression of the exogenous agent is induced. In some embodiments, expression of the exogenous agent is induced immediately prior to generating the lipid particle. In some embodiments, expression of the exogenous agent is induced at the same time as expression of the fusogen.

In some embodiments, the exogenous agent is loaded into the lipid particle via electroporation into the lipid particle itself or into the cell from which the lipid particle is derived. In some embodiments, the exogenous agent is loaded into the lipid particle via transfection (e.g., of a DNA or mRNA encoding the exogenous agent) into the lipid particle itself or into the cell from which the lipid particle is derived.

In some embodiments, the exogenous agent may include one or more nucleic acid sequences, one or more polypeptides, a combination of nucleic acid sequences and/or polypeptides, one or more organelles, and any combination thereof. In some embodiments, the exogenous agent may include one or more cellular components. In some embodiments, the exogenous agent includes one or more cytosolic and/or nuclear components.

In some embodiments, the lipid particle contains an exogenous agent that is a nucleic acid or contains a nucleic acid encoding the exogenous agent. In some embodiments, the nucleic acid is operatively linked to a “positive target cell-specific regulatory element” (or positive TCSRE). In some embodiments, the positive TCSRE is a functional nucleic acid sequence. In some embodiments, the positive TCSRE comprises a promoter or enhancer. In some embodiments, the TCSRE is a nucleic acid sequence that increases the level of an exogenous agent in a target cell. In some embodiments, the positive target cell-specific regulatory element comprises a T cell-specific promoter, a T cell-specific enhancer, a T cell-specific splice site, a T cell-specific site extending half-life of an RNA or protein, a T cell-specific mRNA nuclear export promoting site, a T cell-specific translational enhancing site, or a T cell-specific post-translational modification site. In some embodiments, the T cell-specific promoter is a promoter described in Immgen consortium, herein incorporated by reference in its entirety, e.g., the T cell-specific promoter is an IL2RA (CD25), LRRC32, FOXP3, or IKZF2 promoter. In some embodiments, the T cell-specific promoter or enhancer is a promoter or enhancer described in Schmidl et a, Blood. 2014 Apr. 24; 123 (17): e68-78, herein incorporated by reference in its entirety. In some embodiments, the T cell-specific promoter is a transcriptionally active fragment of any of the foregoing. In some embodiments, the T-cell specific promoter is a variant having at least 70%, 75%, 80%, 85%, 90%, 95%, 96%, 97%, 98%, or 99% identity to any of the foregoing.

In some embodiments, the lipid particle contains an exogenous agent that is a nucleic acid or contains a nucleic acid encoding the exogenous agent. In some embodiments, the nucleic acid is operatively linked to a “negative target cell-specific regulatory element” (or negative TCSRE). In some embodiments, the negative TCSRE is a functional nucleic acid sequence. In some embodiments, the negative TCSRE is a miRNA recognition site that causes degradation of inhibition of the lipid particle in a non-target cell. In some embodiments, the exogenous agent is operatively linked to a “non-target cell-specific regulatory element” (or NTCSRE). In some embodiments, the NTCSRE comprises a nucleic acid sequence that decreases the level of an exogenous agent in a non-target cell compared to in a target cell. In some embodiments, the NTCSRE comprises a non-target cell-specific miRNA recognition sequence, non-target cell-specific protease recognition site, non-target cell-specific ubiquitin ligase site, non-target cell-specific transcriptional repression site, or non-target cell-specific epigenetic repression site. In some embodiments, the NTCSRE comprises a tissue-specific miRNA recognition sequence, tissue-specific protease recognition site, tissue-specific ubiquitin ligase site, tissue-specific transcriptional repression site, or tissue-specific epigenetic repression site. In some embodiments, the NTCSRE comprises a non-target cell-specific miRNA recognition sequence, non-target cell-specific protease recognition site, non-target cell-specific ubiquitin ligase site, non-target cell-specific transcriptional repression site, or non-target cell-specific epigenetic repression site. In some embodiments, the NTCSRE comprises a non-target cell-specific miRNA recognition sequence and the miRNA recognition sequence is able to be bound by one or more of miR3 1, miR363, or miR29c. In some embodiments, the NTCSRE is situated or encoded within a transcribed region encoding the exogenous agent, optionally wherein an RNA produced by the transcribed region comprises the miRNA recognition sequence within a UTR or coding region.

### 1. Nucleic Acids

In some embodiments, the exogenous agent may include a nucleic acid. For example, the exogenous agent may comprise RNA to enhance expression of an endogenous protein, or a siRNA or miRNA that inhibits protein expression of an endogenous protein. For example, the endogenous protein may modulate structure or function in the target cells. In some embodiments, the exogenous agent may include a nucleic acid encoding an engineered protein that modulates structure or function in the target cells. In some embodiments, the exogenous agent is a nucleic acid that targets a transcriptional activator that modulate structure or function in the target cells

In some embodiments, a lipid particle described herein comprises a nucleic acid, e.g., RNA or DNA. In some embodiments, the nucleic acid is, comprises, or consists of one or more natural nucleic acid residues. In some embodiments, the nucleic acid is, comprises, or consists of one or more nucleic acid analogs. In some embodiments, the nucleic acid has a nucleotide sequence that encodes a functional gene product such as an RNA or protein. In some embodiments, the nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, the nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, the nucleic acid is partly or wholly single stranded; in some embodiments, the nucleic acid is partly or wholly double stranded. In some embodiments the nucleic acid has a nucleotide sequence comprising at least one element that encodes, or is the complement of a sequence that encodes, a polypeptide. The nucleic acid may include variants, e.g., having an overall sequence identity with a reference nucleic acid of at least 85%, 86%, 87%, 88%, 89%, 90%, 91%, 92%, 93%, 94%, 95%, 96%, 97%, or 99%. In some embodiments, a variant nucleic acid does not share at least one characteristic sequence element with a reference nucleic acid. In some embodiments, a variant nucleic acid shares one or more of the biological activities of the reference nucleic acid. In some embodiments, a nucleic acid variant has a nucleic acid sequence that is identical to that of the reference but for a small number of sequence alterations at particular positions. In some embodiments, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant nucleic acid comprises about 10, about 9, about 8, about 7, about 6, about 5, about 4, about 3, about 2, or about 1 substituted residue as compared to a reference. In some embodiments, a variant nucleic acid comprises a very small number (e.g., fewer than about 5, about 4, about 3, about 2, or about 1) number of substituted, inserted, or deleted, functional residues that participate in a particular biological activity relative to the reference. In some embodiments, a variant nucleic acid comprises not more than about 15, about 12, about 9, about 3, or about 1 addition or deletion, and, in some embodiments, comprises no additions or deletions, as compared to the reference. In some embodiments, a variant nucleic acid comprises fewer than about 27, about 24, about 21, about 18, about 15, about 12, about 9, about 6, about 3, or fewer than about 9, about 6, about 3, or about 2 additions or deletions as compared to the reference.

In some embodiments, the exogenous agent includes a nucleic acid, e.g., DNA, nDNA (nuclear DNA), mtDNA (mitochondrial DNA), protein coding DNA, gene, operon, chromosome, genome, transposon, retrotransposon, viral genome, intron, exon, modified DNA, mRNA (messenger RNA), tRNA (transfer RNA), modified RNA, microRNA, siRNA (small interfering RNA), tmRNA (transfer messenger RNA), rRNA (ribosomal RNA), mtRNA (mitochondrial RNA), snRNA (small nuclear RNA), small nucleolar RNA (snoRNA), SmY RNA (mRNA trans-splicing RNA), gRNA (guide RNA), TERC (telomerase RNA component), aRNA (antisense RNA), cis-NAT (Cis-natural antisense transcript), CRISPR RNA (crRNA), lncRNA (long noncoding RNA), piRNA (piwi-interacting RNA), shRNA (short hairpin RNA), tasiRNA (trans-acting siRNA), eRNA (enhancer RNA), satellite RNA, pcRNA (protein coding RNA), dsRNA (double stranded RNA), RNAi (interfering RNA), circRNA (circular RNA), reprograming RNAs, aptamers, and any combination thereof. In some embodiments, the nucleic acid is a wild-type nucleic acid. In some embodiments, the protein is a mutant nucleic acid. In some embodiments the nucleic acid is a fusion or chimera of multiple nucleic acid sequences

In embodiments, the nucleic acid encodes one or more (e.g. two or more) inhibitory RNA molecules directed against one or more RNA targets. An inhibitory RNA molecule can be, e.g., a miRNA or an shRNA. In some embodiments, the inhibitory molecule can be a precursor of a miRNA, such as for example, a Pri-miRNA or a Pre-miRNA, or a precursor of an shRNA. In some embodiments, the inhibitory molecule can be an artificially derived miRNA or shRNA. In other embodiments, the inhibitory RNA molecule can be a dsRNA (either transcribed or artificially introduced) that is processed into an siRNA or the siRNA itself. In some embodiments, the inhibitory RNA molecule can be a miRNA or shRNA that has a sequence that is not found in nature, or has at least one functional segment that is not found in nature, or has a combination of functional segments that are not found in nature. In illustrative embodiments, at least one or all of the inhibitory RNA molecules are miR-155. In some embodiments, a retroviral vector described herein encodes two or more inhibitory RNA molecules directed against one or more RNA targets. Two or more inhibitory RNA molecules, in some embodiments, can be directed against different targets. In other embodiments, the two or more inhibitory RNA molecules are directed against the same target. In some embodiments, the exogenous agent comprises a shRNA. A shRNA (short hairpin RNA) can comprise a double-stranded structure that is formed by a single self complementary RNA strand. shRNA constructs can comprise a nucleotide sequence identical to a portion, of either coding or non-coding sequence, of a target gene. RNA sequences with insertions, deletions, and single point mutations relative to the target sequence can also be used. Greater than 90% sequence identity, or even 100% sequence identity, between the inhibitory RNA and the portion of the target gene can be used. In certain embodiments, the length of the duplex-forming portion of an shRNA is at least 20, 21, or 22 nucleotides in length, e.g., corresponding in size to RNA products produced by Dicer-dependent cleavage. In certain embodiments, the shRNA construct is at least 25, 50, 100, 200, 300 or 400 bases in length. In certain embodiments, the shRNA construct is 400-800 bases in length. shRNA constructs are highly tolerant of variation in loop sequence and loop size. In embodiments, a retroviral vector that encodes an siRNA, an miRNA, an shRNA, or a ribozyme comprises one or more regulatory sequences, such as, for example, a strong constitutive pol III, e.g., human U6 snRNA promoter, the mouse U6 snRNA promoter, the human and mouse H 1 RNA promoter and the human tRNA-val promoter, or a strong constitutive pol II promoter.

### a. Polypeptides

In some embodiments, the lipid particle contains a nucleic acid that encodes a protein exogenous agent (also referred to as a “payload gene encoding an exogenous agent.”). In some embodiments, a lipid particle described herein comprises an exogenous agent which is or comprises a protein.

In some embodiments, the protein may include moieties other than amino acids (e.g., may be glycoproteins, proteoglycans, etc.) and/or may be otherwise processed or modified. In some embodiments, the protein can sometimes include more than one polypeptide chain, for example linked by one or more disulfide bonds or associated by other means.

In some embodiments, the protein may contain L-amino acids, D-amino acids, or both and may contain any of a variety of amino acid modifications or analogs. In some embodiments, proteins may comprise natural amino acids, non-natural amino acids, synthetic amino acids, and combinations thereof. In some embodiments, proteins are antibodies, antibody fragments, biologically active portions thereof, and/or characteristic portions thereof. In some embodiments, a polypeptide may include its variants, e.g., having an overall sequence identity with a reference polypeptide of at least 85%, 86%, 87%, 88%, 89%, 90%, 91%, 92%, 93%, 94%, 95%, 96%, 97%, or 99%. In some embodiments, a variant polypeptide does not share at least one characteristic sequence element with a reference polypeptide. In some embodiments, a variant polypeptide shares one or more of the biological activities of the reference polypeptide. In some embodiments, a polypeptide variant has an amino acid sequence that is identical to that of the reference but for a small number of sequence alterations at particular positions. In some embodiments, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide comprises about 10, about 9, about 8, about 7, about 6, about 5, about 4, about 3, about 2, or about 1 substituted residue as compared to a reference. In some embodiments, a variant polypeptide comprises a very small number (e.g., fewer than about 5, about 4, about 3, about 2, or about 1) number of substituted, inserted, or deleted, functional that participate in a particular biological activity relative to the reference. In some embodiments, a variant polypeptide comprises not more than about 5, about 4, about 3, about 2, or about 1 addition or deletion, and, in some embodiments, comprises no additions or deletions, as compared to the reference. In some embodiments, a variant polypeptide comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, the protein includes a polypeptide, e.g., enzymes, structural polypeptides, signaling polypeptides, regulatory polypeptides, transport polypeptides, sensory polypeptides, motor polypeptides, defense polypeptides, storage polypeptides, transcription factors, antibodies, cytokines, hormones, catabolic polypeptides, anabolic polypeptides, proteolytic polypeptides, metabolic polypeptides, kinases, transferases, hydrolases, lyases, isomerases, ligases, enzyme modulator polypeptides, protein binding polypeptides, lipid binding polypeptides, membrane fusion polypeptides, cell differentiation polypeptides, epigenetic polypeptides, cell death polypeptides, nuclear transport polypeptides, nucleic acid binding polypeptides, reprogramming polypeptides, DNA editing polypeptides, DNA repair polypeptides, DNA recombination polypeptides, transposase polypeptides, DNA integration polypeptides, targeted endonucleases (e.g. Zinc-finger nucleases, transcription-activator-like nucleases (TALENs), cas9 and homologs thereof), recombinases, and any combination thereof. In some embodiments, the protein targets a protein in the cell for degradation. In some embodiments, the protein targets a protein in the cell for degradation by localizing the protein to the proteasome. In some embodiments, the protein is a wild-type protein. In some embodiments, the protein is a mutant protein.

Exemplary protein exogenous agents are described in the following subsections. In some embodiments, a lipid particle provided herein can include any of such exogenous agents. In particular embodiments, a lipid particle contains a nucleic acid encoding any of such exogenous agents.

### b. Cytosolic Proteins

In some embodiments, the exogenous agent comprises a cytosolic protein, e.g., a protein that is produced in the recipient cell and localizes to the recipient cell cytoplasm. In some embodiments, the exogenous agent comprises a secreted protein, e.g., a protein that is produced and secreted by the recipient cell. In some embodiments, the exogenous agent comprises a nuclear protein, e.g., a protein that is produced in the recipient cell and is imported to the nucleus of the recipient cell. In some embodiments, the exogenous agent comprises an organellar protein (e.g., a mitochondrial protein), e.g., a protein that is produced in the recipient cell and is imported into an organelle (e.g., a mitochondrial) of the recipient cell. In some embodiments, the protein is a wild-type protein or a mutant protein. In some embodiments the protein is a fusion or chimeric protein.

### c. Membrane Proteins

In some embodiments, the exogenous agent comprises a membrane protein. In some embodiments, the membrane protein comprises a chimeric antigen receptor (CAR), a T cell receptor, an integrin, an ion channel, a pore forming protein, a Toll-Like Receptor, an interleukin receptor, a cell adhesion protein, or a transport protein.

### 1. Chimeric Antigen Receptors (CARs)

In certain embodiments, the payload gene may comprise an exogenous polynucleotide encoding a CAR. CARs (also known as chimeric immunoreceptors, chimeric T cell receptors, or artificial T cell receptors) are receptor proteins that have been engineered to give host cells (e.g., T cells) the new ability to target a specific protein. The receptors are chimeric because they combine both antigen-binding and T cell activating functions into a single receptor. The polycistronic vector of the present disclosure may be used to express one or more CARs in a host cell (e.g., a T cell) for use in cell-based therapies against various target antigens. The CARs expressed by the one or more expression cassettes may be the same or different. In these embodiments, the CAR may comprise an extracellular binding domain (also referred to as a “binder”) that specifically binds a target antigen, a transmembrane domain, and an intracellular signaling domain. In certain embodiments, the CAR may further comprise one or more additional elements, including one or more signal peptides, one or more extracellular hinge domains, and/or one or more intracellular costimulatory domains. Domains may be directly adjacent to one another, or there may be one or more amino acids linking the domains. The nucleotide sequence encoding a CAR may be derived from a mammalian sequence, for example, a mouse sequence, a primate sequence, a human sequence, or combinations thereof. In the cases where the nucleotide sequence encoding a CAR is non-human, the sequence of the CAR may be humanized. The nucleotide sequence encoding a CAR may also be codon-optimized for expression in a mammalian cell, for example, a human cell. In any of these embodiments, the nucleotide sequence encoding a CAR may be at least 80% identical (e.g., at least 80%, at least 85%, at least 90%, at least 95%, at least 96%, at least 97%, at least 98%, at least 99%, or 100% identical) to any of the nucleotide sequences disclosed herein. The sequence variations may be due to codon-optimization, humanization, restriction enzyme-based cloning scars, and/or additional amino acid residues linking the functional domains, etc.

In certain embodiments, the CAR may comprise a signal peptide at the N-terminus. Non-limiting examples of signal peptides include CD8α signal peptide, IgK signal peptide, and granulocyte-macrophage colony-stimulating factor receptor subunit alpha (GMCSFR-α, also known as colony stimulating factor 2 receptor subunit alpha (CSF2RA)) signal peptide, and variants thereof, the amino acid sequences of which are provided in Table 5 below.

TABLE 5
Exemplary sequences of signal peptides

| SEQ ID | | |
|---|---|---|
| NO: | Sequence | Description |
| 378 | MALPVTALLLPLALL | CD8α signal peptide |
| | LHAARP | |
| 379 | METDTLLLWVLLLWV | IgK signal peptide |
| | PGSTG | |
| 380 | MLLLVTSLLLCELPH | GMCSFR-α (CSF2RA) |
| | PAFLLIP | signal peptide |

In certain embodiments, the extracellular binding domain of the CAR may comprise one or more antibodies specific to one target antigen or multiple target antigens. The antibody may be an antibody fragment, for example, an scFv, or a single-domain antibody fragment, for example, a VHH. In certain embodiments, the scFv may comprise a heavy chain variable region (V_{H}) and a light chain variable region (V_{L}) of an antibody connected by a linker. The V_{H} and the V_{L} may be connected in either order, i.e., V_{H}-linker-V_{L} or V_{L}-linker-V_{H}. Non-limiting examples of linkers include Whitlow linker, (G4S)ₙ (n can be a positive integer, e.g., 1, 2, 3, 4, 5, 6, etc.) linker, and variants thereof. In certain embodiments, the antigen may be an antigen that is exclusively or preferentially expressed on tumor cells, or an antigen that is characteristic of an autoimmune or inflammatory disease. Exemplary target antigens include, but are not limited to, CD5, CD19, CD20, CD22, CD23, CD30, CD70, Kappa, Lambda, and B cell maturation agent (BCMA), G-protein coupled receptor family C group 5 member D (GPRC5D) (associated with leukemias); CS1/SLAMF7, CD38, CD138, GPRC5D, TACI, and BCMA (associated with myelomas); GD2, HER2, EGFR, EGFRVIII, B7H3, PSMA, PSCA, CAIX, CD171, CEA, CSPG4, EPHA2, FAP, FRα, IL-13Rα, Mesothelin, MUC1, MUC16, and ROR1 (associated with solid tumors). In any of these embodiments, the extracellular binding domain of the CAR can be codon-optimized for expression in a host cell or have variant sequences to increase functions of the extracellular binding domain.

In certain embodiments, the CAR may comprise a hinge domain, also referred to as a spacer. The terms “hinge” and “spacer” may be used interchangeably in the present disclosure. Non-limiting examples of hinge domains include CD8 hinge domain, CD28 hinge domain, IgG4 hinge domain, IgG4 hinge-CH2-CH3 domain, and variants thereof, the amino acid sequences of which are provided in Table 6 below.

TABLE 6
Exemplary sequences of hinge domains

| SEQ ID | | |
|---|---|---|
| NO: | Sequence | Description |
| 381 | TTTPAPRPPTPAPTIASQP | CD8α hinge domain |
| | LSLRPEACRPAAGGAVHTR | |
| | GLDFACD | |
| 382 | IEVMYPPPYLDNEKSNGTI | CD28 hinge domain |
| | IHVKGKHLCPSPLFPGPSK | |
| | P | |
| 383 | AAAIEVMYPPPYLDNEKSN | CD28 hinge domain |
| | GTIIHVKGKHLCPSPLFPG | |
| | PSKP | |
| 384 | ESKYGPPCPPCP | IgG4 hinge domain |
| 385 | ESKYGPPCPSCP | IgG4 hinge domain |
| 386 | ESKYGPPCPPCPAPEFLGG | IgG4 hinge-CH2-CH3 |
| | PSVFLFPPKPKDTLMISRT | domain |
| | PEVTCVVVDVSQEDPEVQF | |
| | NWYVDGVEVHNAKTKPREE | |
| | QFNSTYRVVSVLTVLHQDW | |
| | LNGKEYKCKVSNKGLPSSI | |
| | EKTISKAKGQPREPQVYTL | |
| | PPSQEEMTKNQVSLTCLVK | |
| | GFYPSDIAVEWESNGQPEN | |
| | NYKTTPPVLDSDGSFFLYS | |
| | RLTVDKSRWQEGNVFSCSV | |
| | MHEALHNHYTQKSLSLSLG | |
| | K | |

In certain embodiments, the transmembrane domain of the CAR may comprise a transmembrane region of the alpha, beta, or zeta chain of a T cell receptor, CD28, CD38, CD45, CD4, CD5, CD8, CD9, CD16, CD22, CD33, CD37, CD64, CD80, CD86, CD134, CD137, CD154, or a functional variant thereof, including the human versions of each of these sequences. In other embodiments, the transmembrane domain may comprise a transmembrane region of CD8a, CD8B, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B, or a functional variant thereof, including the human versions of each of these sequences. Table 7 provides the amino acid sequences of a few exemplary transmembrane domains.

TABLE 7
Exemplary sequences of transmembrane domains

| SEQ ID | | |
|---|---|---|
| NO: | Sequence | Description |
| 387 | IYIWAPLAGTCGVL | CD8α transmembrane |
| | LLSLVITLYC | domain |
| 388 | FWVLVVVGGVLACY | CD28 transmembrane |
| | SLLVTVAFIIFWV | domain |
| 389 | MFWVLVVVGGVLAC | CD28 transmembrane |
| | YSLLVTVAFIIFWV | domain |

In certain embodiments, the intracellular signaling domain and/or intracellular costimulatory domain of the CAR may comprise one or more signaling domains selected from B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, PDCD6, 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNFB, OX40/TNFRSF4, OX40 Ligand/TNFSF4, RELT/TNFRSF19L, TACI/TNFRSF13B, TL1A/TNFSF15, TNFα, TNF RII/TNFRSF1B, 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, SLAM/CD150, CD2, CD7, CD53, CD82/Kai-1, CD90/Thyl, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), NKG2C, CD3ζ, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, and a functional variant thereof including the human versions of each of these sequences. In some embodiments, the intracellular signaling domain and/or intracellular costimulatory domain comprises one or more signaling domains selected from a CD32 domain, an ITAM, a CD28 domain, 4-1BB domain, or a functional variant thereof. Table 8 provides the amino acid sequences of a few exemplary intracellular costimulatory and/or signaling domains. In certain embodiments, as in the case of tisagenlecleucel as described below, the CD3ζ signaling domain of SEQ ID NO:392 may have a mutation, e.g., a glutamine (Q) to lysine (K) mutation, at amino acid position 14 (see SEQ ID NO:393).

TABLE 8
Exemplary sequences of intracellular
costimulatory and/or signaling domains

| SEQ ID | | |
|---|---|---|
| NO: | Sequence | Description |
| 390 | KRGRKKLLYIFKQPFMR | 4-1BB costimulatory |
| | PVQTTQEEDGCSCRFPE | domain |
| | EEEGGCEL | |
| 391 | RSKRSRLLHSDYMNMTP | CD28 costimulatory |
| | RRPGPTRKHYQPYAPPR | domain |
| | DFAAYRS | |
| 392 | RVKFSRSADAPAYQQGQ | CD3ζ signaling |
| | NQLYNELNLGRREEYDV | domain |
| | LDKRRGRDPEMGGKPRR | |
| | KNPQEGLYNELQKDKMA | |
| | EAYSEIGMKGERRRGKG | |
| | HDGLYQGLSTATKDTYD | |
| | ALHMQALPPR | |
| 393 | RVKFSRSADAPAYKQGQ | CD3ζ signaling |
| | NQLYNELNLGRREEYDV | domain (with Q |
| | LDKRRGRDPEMGGKPRR | to K mutation at |
| | KNPQEGLYNELQKDKMA | position 14) |
| | EAYSEIGMKGERRRGKG | |
| | HDGLYQGLSTATKDTYD | |
| | ALHMQALPPR | |

In certain embodiments where the polycistronic vector encodes two or more CARs, the two or more CARs may comprise the same functional domains, or one or more different functional domains, as described. For example, the two or more CARs may comprise different signal peptides, extracellular binding domains, hinge domains, transmembrane domains, costimulatory domains, and/or intracellular signaling domains, in order to minimize the risk of recombination due to sequence similarities. Or, alternatively, the two or more CARs may comprise the same domains. In the cases where the same domain(s) and/or backbone are used, it is optional to introduce codon divergence at the nucleotide sequence level to minimize the risk of recombination.

### 1) CD19 CAR

In some embodiments, the CAR is a CD19 CAR (“CD19-CAR”), and in these embodiments, the polycistronic vector comprises an expression cassette that contains a nucleotide sequence encoding a CD19 CAR. In some embodiments, the CD19 CAR may comprise a signal peptide, an extracellular binding domain that specifically binds CD19, a hinge domain, a transmembrane domain, an intracellular costimulatory domain, and/or an intracellular signaling domain in tandem.

In some embodiments, the signal peptide of the CD19 CAR comprises a CD8α signal peptide. In some embodiments, the CD8α signal peptide comprises or consists of an amino acid sequence set forth in SEQ ID NO:378 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 378. In some embodiments, the signal peptide comprises an IgK signal peptide. In some embodiments, the IgK signal peptide comprises or consists of an amino acid sequence set forth in SEQ ID NO:379 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 379. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:380 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 380.

In some embodiments, the extracellular binding domain of the CD19 CAR is specific to CD19, for example, human CD19. The extracellular binding domain of the CD19 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD19 CAR comprises an scFv derived from the FMC63 monoclonal antibody (FMC63), which comprises the heavy chain variable region (V_{H}) and the light chain variable region (V_{L}) of FMC63 connected by a linker. FMC63 and the derived scFv have been described in Nicholson et al., Mol. Immun. 34 (16-17): 1157-1165 (1997) and PCT Application Publication No. WO2018/213337, the entire contents of each of which are incorporated by reference herein. In some embodiments, the amino acid sequences of the entire FMC63-derived scFv (also referred to as FMC63 scFv) and its different portions are provided in Table 9 below. In some embodiments, the CD19-specific scFv comprises or consists of an amino acid sequence set forth in SEQ ID NO: 394, 395, or 400, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:40, 41, or 46. In some embodiments, the CD19-specific scFv may comprise one or more CDRs having amino acid sequences set forth in SEQ ID NOs: 42-44 and 48-50. In some embodiments, the CD19-specific scFv may comprise a light chain with one or more CDRs having amino acid sequences set forth in SEQ ID NOs: 396-398. In some embodiments, the CD19-specific scFv may comprise a heavy chain with one or more CDRs having amino acid sequences set forth in SEQ ID NOs: 402-404. In any of these embodiments, the CD19-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD19 CAR comprises or consists of the one or more CDRs as described herein.

In some embodiments, the linker linking the V_{H} and the V_{L} portions of the scFv is a Whitlow linker having an amino acid sequence set forth in SEQ ID NO:399. In some embodiments, the Whitlow linker may be replaced by a different linker, for example, a 3×G₄S linker having an amino acid sequence set forth in SEQ ID NO:405, which gives rise to a different FMC63-derived scFv having an amino acid sequence set forth in SEQ ID NO:404. In certain of these embodiments, the CD19-specific scFv comprises or consists of an amino acid sequence set forth in SEQ ID NO:404 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 404.

TABLE 9
Exemplary sequences
of anti-CD19 scFv and components

| SEQ | | |
|---|---|---|
| ID NO: | Amino Acid Sequence | Description |
| 394 | DIQMTQTTSSLSASLGDRVTISCRASQDI | Anti-CD19 FMC63 scFv |
| | SKYLNWYQQKPDGTVKLLIYHTSRLHS | entire sequence, with |
| | GVPSRFSGSGSGTDYSLTISNLEQEDIAT | Whitlow linker |
| | YFCQQGNTLPYTFGGGTKLEITGSTSGS | |
| | GKPGSGEGSTKGEVKLQESGPGLVAPS | |
| | QSLSVTCTVSGVSLPDYGVSWIRQPPRK | |
| | GLEWLGVIWGSETTYYNSALKSRLTIIK | |
| | DNSKSQVFLKMNSLQTDDTAIYYCAKH | |
| | YYYGGSYAMDYWGQGTSVTVSS | |
| 395 | DIQMTQTTSSLSASLGDRVTISCRASQDI | Anti-CD19 FMC63 scFv |
| | SKYLNWYQQKPDGTVKLLIYHTSRLHS | light chain variable |
| | GVPSRFSGSGSGTDYSLTISNLEQEDIAT | region |
| | YFCQQGNTLPYTFGGGTKLEIT | |
| 396 | QDISKY | Anti-CD19 FMC63 scFv |
| | | light chain CDR1 |
| 397 | HTS | Anti-CD19 FMC63 scFv |
| | | light chain CDR2 |
| 398 | QQGNTLPYT | Anti-CD19 FMC63 scFv |
| | | light chain CDR3 |
| 399 | GSTSGSGKPGSGEGSTKG | Whitlow linker |
| 400 | EVKLQESGPGLVAPSQSLSVTCTVSGVS | Anti-CD19 FMC63 scFv |
| | LPDYGVSWIRQPPRKGLEWLGVIWGSE | heavy chain variable |
| | TTYYNSALKSRLTIIKDNSKSQVFLKMN | region |
| | SLQTDDTAIYYCAKHYYYGGSYAMDY | |
| | WGQGTSVTVSS | |
| 401 | GVSLPDYG | Anti-CD19 FMC63 scFv |
| | | heavy chain CDR1 |
| 402 | IWGSETT | Anti-CD19 FMC63 scFv |
| | | heavy chain CDR2 |
| 403 | AKHYYYGGSYAMDY | Anti-CD19 FMC63 scFv |
| | | heavy chain CDR3 |
| 404 | DIQMTQTTSSLSASLGDRVTISCRASQDI | Anti-CD19 FMC63 scFv |
| | SKYLNWYQQKPDGTVKLLIYHTSRLHS | entire sequence, with |
| | GVPSRFSGSGSGTDYSLTISNLEQEDIAT | 3xG₄S linker |
| | YFCQQGNTLPYTFGGGTKLEITGGGGS | |
| | GGGGSGGGGSEVKLQESGPGLVAPSQS | |
| | LSVTCTVSGVSLPDYGVSWIRQPPRKGL | |
| | EWLGVIWGSETTYYNSALKSRLTIIKDN | |
| | SKSQVFLKMNSLQTDDTAIYYCAKHYY | |
| | YGGSYAMDYWGQGTSVTVSS | |
| 405 | GGGGSGGGGSGGGGS | 3xG₄S linker |

In some embodiments, the extracellular binding domain of the CD19 CAR is derived from an antibody specific to CD19, including, for example, SJ25C1 (Bejcek et al., Cancer Res. 55:2346-2351 (1995)), HD37 (Pezutto et al., J. Immunol. 138 (9): 2793-2799 (1987)), 4G7 (Meeker et al., Hybridoma 3:305-320 (1984)), B43 (Bejcek (1995)), BLY3 (Bejcek (1995)), B4 (Freedman et al., 70:418-427 (1987)), B4 HB12b (Kansas & Tedder, J. Immunol. 147:4094-4102 (1991); Yazawa et al., Proc. Natl. Acad. Sci. USA 102:15178-15183 (2005); Herbst et al., J. Pharmacol. Exp. Ther. 335:213-222 (2010)), BU12 (Callard et al., J. Immunology, 148 (10): 2983-2987 (1992)), and CLB-CD19 (De Rie Cell. Immunol. 118:368-381 (1989)). In any of these embodiments, the extracellular binding domain of the CD19 CAR can comprise or consist of the V_{H}, the V_{L}, and/or one or more CDRs of any of the antibodies.

In some embodiments, the hinge domain of the CD19 CAR comprises a CD8α hinge domain, for example, a human CD8 hinge domain. In some embodiments, the CD8α hinge domain comprises or consists of an amino acid sequence set forth in SEQ ID NO:381 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:381. In some embodiments, the hinge domain comprises a CD28 hinge domain, for example, a human CD28 hinge domain. In some embodiments, the CD28 hinge domain comprises or consists of an amino acid sequence set forth in SEQ ID NO:382 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:382. In some embodiments, the CD28 hinge domain comprises or consists of an amino acid sequence set forth in SEQ ID NO:383 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:383. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:384 or SEQ ID NO:385, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:384 or SEQ ID NO:385. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:386 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:386.

In some embodiments, the transmembrane domain of the CD19 CAR comprises a CD8α transmembrane domain, for example, a human CD8α transmembrane domain. In some embodiments, the CD8α transmembrane domain comprises or consists of an amino acid sequence set forth in SEQ ID NO: 387 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:387. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:388 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:388. In some embodiments, the CD28 transmembrane domain comprises or consists of an amino acid sequence set forth in SEQ ID NO:389 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:389.

In some embodiments, the intracellular costimulatory domain of the CD19 CAR comprises a 4-1BB costimulatory domain. 4-1BB, also known as CD137, transmits a potent costimulatory signal to T cells, promoting differentiation and enhancing long-term survival of T lymphocytes. In some embodiments, the 4-1BB costimulatory domain is human. In some embodiments, the 4-1BB costimulatory domain comprises or consists of an amino acid sequence set forth in SEQ ID NO:390 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:390. In some embodiments, the intracellular costimulatory domain comprises a CD28 costimulatory domain. CD28 is another co-stimulatory molecule on T cells. In some embodiments, the CD28 costimulatory domain is human. In some embodiments, the CD28 costimulatory domain comprises or consists of an amino acid sequence set forth in SEQ ID NO:391 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:391. In some embodiments, the intracellular costimulatory domain of the CD19 CAR comprises a 4-1BB costimulatory domain and a CD28 costimulatory domain as described.

In some embodiments, the intracellular signaling domain of the CD19 CAR comprises a CD3 zeta (ζ) signaling domain. CD3 associates with T cell receptors (TCRs) to produce a signal and contains immunoreceptor tyrosine-based activation motifs (ITAMs). The CD3ζ signaling domain refers to amino acid residues from the cytoplasmic domain of the zeta chain that are sufficient to functionally transmit an initial signal necessary for T cell activation. In some embodiments, the CD3ζ signaling domain is human. In some embodiments, the CD3 signaling domain comprises or consists of an amino acid sequence set forth in SEQ ID NO:392 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:392.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO:394 or SEQ ID NO:395, the CD8α hinge domain of SEQ ID NO:381, the CD8α transmembrane domain of SEQ ID NO:387, the 4-1BB costimulatory domain of SEQ ID NO:390, the CD3ζ signaling domain of SEQ ID NO:392, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO:394 or SEQ ID NO:395, the IgG4 hinge domain of SEQ ID NO:384 or SEQ ID NO:385, the CD28 transmembrane domain of SEQ ID NO: 388, the 4-1BB costimulatory domain of SEQ ID NO:390, the CD3ζ signaling domain of SEQ ID NO: 392, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO:394 or SEQ ID NO:395, the CD28 hinge domain of SEQ ID NO:383, the CD28 transmembrane domain of SEQ ID NO:388, the CD28 costimulatory domain of SEQ ID NO:391, the CD3ζ signaling domain of SEQ ID NO:392, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR as set forth in SEQ ID NO:406 or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:406 (see Table 11). The encoded CD19 CAR has a corresponding amino acid sequence set forth in SEQ ID NO:407 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 407, with the following components: CD8α signal peptide, FMC63 scFv (V_{L}-Whitlow linker-V_{H}), CD8α hinge domain, CD8α transmembrane domain, 4-1BB costimulatory domain, and CD3ζ signaling domain.

In some embodiments, the polycistronic vector comprises an expression cassette that contains a nucleotide sequence encoding a commercially available embodiment of CD19 CAR. Non-limiting examples of commercially available embodiments of CD19 CARs expressed and/or encoded by T cells include tisagenlecleucel, lisocabtagene maraleucel, axicabtagene ciloleucel, and brexucabtagene autoleucel.

In some embodiments, the polycistronic vector comprises an expression cassette that contains a nucleotide sequence encoding tisagenlecleucel or portions thereof. Tisagenlecleucel comprises a CD19 CAR with the following components: CD8α signal peptide, FMC63 scFv (V_{L}-3×G₄S linker-V_{H}), CD8α hinge domain, CD8α transmembrane domain, 4-1BB costimulatory domain, and CD3ζ signaling domain. The nucleotide and amino acid sequence of the CD19 CAR in tisagenlecleucel are provided in Table 10, with annotations of the sequences provided in Table 11.

In some embodiments, the polycistronic vector comprises an expression cassette that contains a nucleotide sequence encoding lisocabtagene maraleucel or portions thereof. Lisocabtagene maraleucel comprises a CD19 CAR with the following components: GMCSFR-α or CSF2RA signal peptide, FMC63 scFv (V_{L}-Whitlow linker-V_{H}), IgG4 hinge domain, CD28 transmembrane domain, 4-1BB costimulatory domain, and CD3ζ signaling domain. The nucleotide and amino acid sequence of the CD19 CAR in lisocabtagene maraleucel are provided in Table 10, with annotations of the sequences provided in Table 12.

In some embodiments, the polycistronic vector comprises an expression cassette that contains a nucleotide sequence encoding axicabtagene ciloleucel or portions thereof. Axicabtagene ciloleucel comprises a CD19 CAR with the following components: GMCSFR-α or CSF2RA signal peptide, FMC63 scFv (V_{L}-Whitlow linker-V_{H}), CD28 hinge domain, CD28 transmembrane domain, CD28 costimulatory domain, and CD3ζ signaling domain. The nucleotide and amino acid sequence of the CD19 CAR in axicabtagene ciloleucel are provided in Table 10, with annotations of the sequences provided in Table 13.

In some embodiments, the polycistronic vector comprises an expression cassette that contains a nucleotide sequence encoding brexucabtagene autoleucel or portions thereof. Brexucabtagene autoleucel comprises a CD19 CAR with the following components: GMCSFR-α signal peptide, FMC63 scFv, CD28 hinge domain, CD28 transmembrane domain, CD28 costimulatory domain, and CD3ζ signaling domain.

In some embodiments, the polycistronic vector comprises an expression cassette that contains a nucleotide sequence encoding a CD19 CAR as set forth in SEQ ID NO: 408, 410, or 412, or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 408, 410, or 412. The encoded CD19 CAR has a corresponding amino acid sequence set forth in SEQ ID NO: 409, 411, or 413, respectively, or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 409, 411, or 413, respectively.

TABLE 10
Exemplary sequences of CD19 CARs

| SEQ | | |
|---|---|---|
| ID NO: | Sequence | Description |
| 406 | atggccttaccagtgaccgccttgctcctgccgctggccttgctgctccac | Exemplary CD19 |
| | gccgccaggccggacatccagatgacacagactacatcctccctgtctgc | CAR nucleotide |
| | ctctctgggagacagagtcaccatcagttgcagggcaagtcaggacatta | sequence |
| | gtaaatatttaaattggtatcagcagaaaccagatggaactgttaaactcct | |
| | gatctaccatacatcaagattacactcaggagtcccatcaaggttcagtgg | |
| | cagtgggtctggaacagattattctctcaccattagcaacctggagcaaga | |
| | agatattgccacttacttttgccaacagggtaatacgcttccgtacacgttcg | |
| | gaggggggaccaagctggagatcacaggctccacctctggatccggca | |
| | agcccggatctggcgagggatccaccaagggcgaggtgaaactgcag | |
| | gagtcaggacctggcctggtggcgccctcacagagcctgtccgtcacat | |
| | gcactgtctcaggggtctcattacccgactatggtgtaagctggattcgcc | |
| | agcctccacgaaagggtctggagtggctgggagtaatatggggtagtga | |
| | aaccacatactataattcagctctcaaatccagactgaccatcatcaagga | |
| | caactccaagagccaagttttcttaaaaatgaacagtctgcaaactgatga | |
| | cacagccatttactactgtgccaaacattattactacggtggtagctatgcta | |
| | tggactactggggccaaggaacctcagtcaccgtctcctcaaccacgac | |
| | gccagcgccgcgaccaccaacaccggcgcccaccatcgcgtcgcagc | |
| | ccctgtccctgcgcccagaggcgtgccggccagcggcggggggcgca | |
| | gtgcacacgagggggctggacttcgcctgtgatatctacatctgggcgcc | |
| | cttggccgggacttgtggggtccttctcctgtcactggttatcaccctttact | |
| | gcaaacggggcagaaagaaactcctgtatatattcaaacaaccatttatga | |
| | gaccagtacaaactactcaagaggaagatggctgtagctgccgatttcca | |
| | gaagaagaagaaggaggatgtgaactgagagtgaagttcagcaggagc | |
| | gcagacgcccccgcgtaccagcagggccagaaccagctctataacgag | |
| | ctcaatctaggacgaagagaggagtacgatgttttggacaagagacgtgg | |
| | ccgggaccctgagatggggggaaagccgagaaggaagaaccctcagg | |
| | aaggcctgtacaatgaactgcagaaagataagatggcggaggcctacag | |
| | tgagattgggatgaaaggcgagcgccggaggggcaaggggcacgatg | |
| | gcctttaccagggtctcagtacagccaccaaggacacctacgacgccctt | |
| | cacatgcaggccctgccccctcgc | |
| 407 | MALPVTALLLPLALLLHAARPDIQMTQTTSSLS | Exemplary CD19 |
| | ASLGDRVTISCRASQDISKYLNWYQQKPDGTV | CAR amino acid |
| | KLLIYHTSRLHSGVPSRFSGSGSGTDYSLTISNL | sequence |
| | EQEDIATYFCQQGNTLPYTFGGGTKLEITGSTS | |
| | GSGKPGSGEGSTKGEVKLQESGPGLVAPSQSLS | |
| | VTCTVSGVSLPDYGVSWIRQPPRKGLEWLGVI | |
| | WGSETTYYNSALKSRLTIIKDNSKSQVFLKMNS | |
| | LQTDDTAIYYCAKHYYYGGSYAMDYWGQGT | |
| | SVTVSSTTTPAPRPPTPAPTIASQPLSLRPEACRP | |
| | AAGGAVHTRGLDFACDIYIWAPLAGTCGVLLL | |
| | SLVITLYCKRGRKKLLYIFKQPFMRPVQTTQEE | |
| | DGCSCRFPEEEEGGCELRVKFSRSADAPAYQQ | |
| | GQNQLYNELNLGRREEYDVLDKRRGRDPEMG | |
| | GKPRRKNPQEGLYNELQKDKMAEAYSEIGMK | |
| | GERRRGKGHDGLYQGLSTATKDTYDALHMQA | |
| | LPPR | |
| 408 | atggccttaccagtgaccgccttgctcctgccgctggccttgctgctccac | Tisagenlecleucel |
| | gccgccaggccggacatccagatgacacagactacatcctccctgtctgc | CD19 CAR |
| | ctctctgggagacagagtcaccatcagttgcagggcaagtcaggacatta | nucleotide |
| | gtaaatatttaaattggtatcagcagaaaccagatggaactgttaaactcct | sequence |
| | gatctaccatacatcaagattacactcaggagtcccatcaaggttcagtgg | |
| | cagtgggtctggaacagattattctctcaccattagcaacctggagcaaga | |
| | agatattgccacttacttttgccaacagggtaatacgcttccgtacacgttcg | |
| | gaggggggaccaagctggagatcacaggtggcggtggctcgggcggt | |
| | ggtgggtcgggtggcggcggatctgaggtgaaactgcaggagtcagga | |
| | cctggcctggtggcgccctcacagagcctgtccgtcacatgcactgtctc | |
| | aggggtctcattacccgactatggtgtaagctggattcgccagcctccacg | |
| | aaagggtctggagtggctgggagtaatatggggtagtgaaaccacatact | |
| | ataattcagctctcaaatccagactgaccatcatcaaggacaactccaaga | |
| | gccaagttttcttaaaaatgaacagtctgcaaactgatgacacagccattta | |
| | ctactgtgccaaacattattactacggtggtagctatgctatggactactgg | |
| | ggccaaggaacctcagtcaccgtctcctcaaccacgacgccagcgccg | |
| | cgaccaccaacaccggcgcccaccatcgcgtcgcagcccctgtccctgc | |
| | gcccagaggcgtgccggccagcggcggggggcgcagtgcacacgag | |
| | ggggctggacttcgcctgtgatatctacatctgggcgcccttggccggga | |
| | cttgtggggtccttctcctgtcactggttatcaccctttactgcaaacggggc | |
| | agaaagaaactcctgtatatattcaaacaaccatttatgagaccagtacaaa | |
| | ctactcaagaggaagatggctgtagctgccgatttccagaagaagaagaa | |
| | ggaggatgtgaactgagagtgaagttcagcaggagcgcagacgccccc | |
| | gcgtacaagcagggccagaaccagctctataacgagctcaatctaggac | |
| | gaagagaggagtacgatgttttggacaagagacgtggccgggaccctga | |
| | gatggggggaaagccgagaaggaagaaccctcaggaaggcctgtaca | |
| | atgaactgcagaaagataagatggcggaggcctacagtgagattgggat | |
| | gaaaggcgagcgccggaggggcaaggggcacgatggcctttaccagg | |
| | gtctcagtacagccaccaaggacacctacgacgcccttcacatgcaggc | |
| | cctgccccctcgc | |
| 409 | MALPVTALLLPLALLLHAARPDIQMTQTTSSLS | Tisagenlecleucel |
| | ASLGDRVTISCRASQDISKYLNWYQQKPDGTV | CD19 CAR amino |
| | KLLIYHTSRLHSGVPSRFSGSGSGTDYSLTISNL | acid sequence |
| | EQEDIATYFCQQGNTLPYTFGGGTKLEITGGGG | |
| | SGGGGSGGGGSEVKLQESGPGLVAPSQSLSVT | |
| | CTVSGVSLPDYGVSWIRQPPRKGLEWLGVIWG | |
| | SETTYYNSALKSRLTIIKDNSKSQVFLKMNSLQ | |
| | TDDTAIYYCAKHYYYGGSYAMDYWGQGTSV | |
| | TVSSTTTPAPRPPTPAPTIASQPLSLRPEACRPA | |
| | AGGAVHTRGLDFACDIYIWAPLAGTCGVLLLS | |
| | LVITLYCKRGRKKLLYIFKQPFMRPVQTTQEED | |
| | GCSCRFPEEEEGGCELRVKFSRSADAPAYKQG | |
| | QNQLYNELNLGRREEYDVLDKRRGRDPEMGG | |
| | KPRRKNPQEGLYNELQKDKMAEAYSEIGMKG | |
| | ERRRGKGHDGLYQGLSTATKDTYDALHMQAL | |
| | PPR | |
| 410 | atgctgctgctggtgaccagcctgctgctgtgcgagctgccccaccccgc | Lisocabtagene |
| | ctttctgctgatccccgacatccagatgacccagaccacctccagcctgag | maraleucel CD19 |
| | cgccagcctgggcgaccgggtgaccatcagctgccgggccagccagg | CAR nucleotide |
| | acatcagcaagtacctgaactggtatcagcagaagcccgacggcaccgt | sequence |
| | caagctgctgatctaccacaccagccggctgcacagcggcgtgcccagc | |
| | cggtttagcggcagcggctccggcaccgactacagcctgaccatctcca | |
| | acctggaacaggaagatatcgccacctacttttgccagcagggcaacaca | |
| | ctgccctacacctttggcggcggaacaaagctggaaatcaccggcagca | |
| | cctccggcagcggcaagcctggcagcggcgagggcagcaccaaggg | |
| | cgaggtgaagctgcaggaaagcggccctggcctggtggcccccagcca | |
| | gagcctgagcgtgacctgcaccgtgagcggcgtgagcctgcccgacta | |
| | cggcgtgagctggatccggcagccccccaggaagggcctggaatggct | |
| | gggcgtgatctggggcagcgagaccacctactacaacagcgccctgaa | |
| | gagccggctgaccatcatcaaggacaacagcaagagccaggtgttcctg | |
| | aagatgaacagcctgcagaccgacgacaccgccatctactactgcgcca | |
| | agcactactactacggcggcagctacgccatggactactggggccaggg | |
| | caccagcgtgaccgtgagcagcgaatctaagtacggaccgccctgcccc | |
| | ccttgccctatgttctgggtgctggtggtggtcggaggcgtgctggcctgc | |
| | tacagcctgctggtcaccgtggccttcatcatcttttgggtgaaacggggc | |
| | agaaagaaactcctgtatatattcaaacaaccatttatgagaccagtacaaa | |
| | ctactcaagaggaagatggctgtagctgccgatttccagaagaagaagaa | |
| | ggaggatgtgaactgcgggtgaagttcagcagaagcgccgacgcccct | |
| | gcctaccagcagggccagaatcagctgtacaacgagctgaacctgggc | |
| | agaagggaagagtacgacgtcctggataagcggagaggccgggaccc | |
| | tgagatgggcggcaagcctcggcggaagaacccccaggaaggcctgta | |
| | taacgaactgcagaaagacaagatggccgaggcctacagcgagatcgg | |
| | catgaagggcgagcggaggcggggcaagggccacgacggcctgtatc | |
| | agggcctgtccaccgccaccaaggatacctacgacgccctgcacatgca | |
| | ggccctgcccccaagg | |
| 411 | MLLLVTSLLLCELPHPAFLLIPDIQMTQTTSSLS | Lisocabtagene |
| | ASLGDRVTISCRASQDISKYLNWYQQKPDGTV | maraleucel CD19 |
| | KLLIYHTSRLHSGVPSRFSGSGSGTDYSLTISNL | CAR amino acid |
| | EQEDIATYFCQQGNTLPYTFGGGTKLEITGSTS | sequence |
| | GSGKPGSGEGSTKGEVKLQESGPGLVAPSQSLS | |
| | VTCTVSGVSLPDYGVSWIRQPPRKGLEWLGVI | |
| | WGSETTYYNSALKSRLTIIKDNSKSQVFLKMNS | |
| | LQTDDTAIYYCAKHYYYGGSYAMDYWGQGT | |
| | SVTVSSESKYGPPCPPCPMFWVLVVVGGVLAC | |
| | YSLLVTVAFIIFWVKRGRKKLLYIFKQPFMRPV | |
| | QTTQEEDGCSCRFPEEEEGGCELRVKFSRSADA | |
| | PAYQQGQNQLYNELNLGRREEYDVLDKRRGR | |
| | DPEMGGKPRRKNPQEGLYNELQKDKMAEAYS | |
| | EIGMKGERRRGKGHDGLYQGLSTATKDTYDA | |
| | LHMQALPPR | |
| 412 | atgcttctcctggtgacaagccttctgctctgtgagttaccacacccagcatt | Axicabtagene |
| | cctcctgatcccagacatccagatgacacagactacatcctccctgtctgc | ciloleucel CD19 |
| | ctctctgggagacagagtcaccatcagttgcagggcaagtcaggacatta | CAR nucleotide |
| | gtaaatatttaaattggtatcagcagaaaccagatggaactgttaaactcct | sequence |
| | gatctaccatacatcaagattacactcaggagtcccatcaaggttcagtgg | |
| | cagtgggtctggaacagattattctctcaccattagcaacctggagcaaga | |
| | agatattgccacttacttttgccaacagggtaatacgcttccgtacacgttcg | |
| | gaggggggactaagttggaaataacaggctccacctctggatccggcaa | |
| | gcccggatctggcgagggatccaccaagggcgaggtgaaactgcagg | |
| | agtcaggacctggcctggtggcgccctcacagagcctgtccgtcacatg | |
| | cactgtctcaggggtctcattacccgactatggtgtaagctggattcgcca | |
| | gcctccacgaaagggtctggagtggctgggagtaatatggggtagtgaa | |
| | accacatactataattcagctctcaaatccagactgaccatcatcaaggac | |
| | aactccaagagccaagttttcttaaaaatgaacagtctgcaaactgatgac | |
| | acagccatttactactgtgccaaacattattactacggtggtagctatgctat | |
| | ggactactggggtcaaggaacctcagtcaccgtctcctcagcggccgca | |
| | attgaagttatgtatcctcctccttacctagacaatgagaagagcaatggaa | |
| | ccattatccatgtgaaagggaaacacctttgtccaagtcccctatttcccgg | |
| | accttctaagcccttttgggtgctggtggtggttgggggagtcctggcttgc | |
| | tatagcttgctagtaacagtggcctttattattttctgggtgaggagtaagag | |
| | gagcaggctcctgcacagtgactacatgaacatgactccccgccgcccc | |
| | gggcccacccgcaagcattaccagccctatgccccaccacgcgacttcg | |
| | cagcctatcgctccagagtgaagttcagcaggagcgcagacgcccccg | |
| | cgtaccagcagggccagaaccagctctataacgagctcaatctaggacg | |
| | aagagaggagtacgatgttttggacaagagacgtggccgggaccctgag | |
| | atggggggaaagccgagaaggaagaaccctcaggaaggcctgtacaat | |
| | gaactgcagaaagataagatggcggaggcctacagtgagattgggatga | |
| | aaggcgagcgccggaggggcaaggggcacgatggcctttaccagggt | |
| | ctcagtacagccaccaaggacacctacgacgcccttcacatgcaggccc | |
| | tgccccctcgc | |
| 413 | MLLLVTSLLLCELPHPAFLLIPDIQMTQTTSSLS | Axicabtagene |
| | ASLGDRVTISCRASQDISKYLNWYQQKPDGTV | ciloleucel CD19 |
| | KLLIYHTSRLHSGVPSRFSGSGSGTDYSLTISNL | CAR amino acid |
| | EQEDIATYFCQQGNTLPYTFGGGTKLEITGSTS | sequence |
| | GSGKPGSGEGSTKGEVKLQESGPGLVAPSQSLS | |
| | VTCTVSGVSLPDYGVSWIRQPPRKGLEWLGVI | |
| | WGSETTYYNSALKSRLTIIKDNSKSQVFLKMNS | |
| | LQTDDTAIYYCAKHYYYGGSYAMDYWGQGT | |
| | SVTVSSAAAIEVMYPPPYLDNEKSNGTIIHVKG | |
| | KHLCPSPLFPGPSKPFWVLVVVGGVLACYSLL | |
| | VTVAFIIFWVRSKRSRLLHSDYMNMTPRRPGPT | |
| | RKHYQPYAPPRDFAAYRSRVKFSRSADAPAYQ | |
| | QGQNQLYNELNLGRREEYDVLDKRRGRDPEM | |
| | GGKPRRKNPQEGLYNELQKDKMAEAYSEIGM | |
| | KGERRRGKGHDGLYQGLSTATKDTYDALHMQ | |
| | ALPPR | |

TABLE 11
Annotation of tisagenlecleucel CD19 CAR sequences

| | Nucleotide | Amino Acid |
|---|---|---|
| | Sequence | Sequence |
| Feature | Position | Position |
| CD8α signal peptide | 1-63 | 1-21 |
| FMC63 scFv | 64-789 | 22-263 |
| (V_{L}-3xG₄S linker-V_{H}) | | |
| CD8α hinge domain | 790-924 | 264-308 |
| CD8α transmembrane domain | 925-996 | 309-332 |
| 4-1BB costimulatory domain | 997-1122 | 333-374 |
| CD3ζ signaling domain | 1123-1458 | 375-486 |

TABLE 12
Annotation of lisocabtagene maraleucel CD19 CAR sequences

| | Nucleotide | Amino Acid |
|---|---|---|
| | Sequence | Sequence |
| Feature | Position | Position |
| GMCSFR-α signal peptide | 1-66 | 1-22 |
| FMC63 scFv | 67-801 | 23-267 |
| (V_{L}-Whitlow linker-V_{H}) | | |
| IgG4 hinge domain | 802-837 | 268-279 |
| CD28 transmembrane domain | 838-921 | 280-307 |
| 4-1BB costimulatory domain | 922-1047 | 308-349 |
| CD3ζ signaling domain | 1048-1383 | 350-461 |

TABLE 13
Annotation of axicabtagene ciloleucel CD19 CAR sequences

| | Nucleotide | Amino Acid |
|---|---|---|
| | Sequence | Sequence |
| Feature | Position | Position |
| CSF2RA signal peptide | 1-66 | 1-22 |
| FMC63 scFv | 67-801 | 23-267 |
| (V_{L}-Whitlow linker-V_{H}) | | |
| CD28 hinge domain | 802-927 | 268-309 |
| CD28 transmembrane domain | 928-1008 | 310-336 |
| CD28 costimulatory domain | 1009-1131 | 337-377 |
| CD3ζ signaling domain | 1132-1467 | 378-489 |

In some embodiments, the polycistronic vector comprises an expression cassette that contains a nucleotide sequence encoding CD19 CAR as set forth in SEQ ID NO: 408, 410, or 412, or at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 408, 410, or 412. The encoded CD19 CAR has a corresponding amino acid sequence set forth in SEQ ID NO: 409, 411, or 412, respectively, is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 409, 411, or 412, respectively.

### 2) CD20 CAR

In some embodiments, the CAR is a CD20 CAR (“CD20-CAR”), and in these embodiments, the polycistronic vector comprises an expression cassette that contains a nucleotide sequence encoding a CD20 CAR. CD20 is an antigen found on the surface of B cells as early at the pro-B phase and progressively at increasing levels until B cell maturity, as well as on the cells of most B-cell neoplasms. CD20 positive cells are also sometimes found in cases of Hodgkin's disease, myeloma, and thymoma. In some embodiments, the CD20 CAR may comprise a signal peptide, an extracellular binding domain that specifically binds CD20, a hinge domain, a transmembrane domain, an intracellular costimulatory domain, and/or an intracellular signaling domain in tandem.

In some embodiments, the signal peptide of the CD20 CAR comprises a CD8α signal peptide. In some embodiments, the CD8α signal peptide comprises or consists of an amino acid sequence set forth in SEQ ID NO:378 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:378. In some embodiments, the signal peptide comprises an IgK signal peptide. In some embodiments, the IgK signal peptide comprises or consists of an amino acid sequence set forth in SEQ ID NO:379 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 379. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:380 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:380.

In some embodiments, the extracellular binding domain of the CD20 CAR is specific to CD20, for example, human CD20. The extracellular binding domain of the CD20 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD20 CAR is derived from an antibody specific to CD20, including, for example, Leu16, IF5, 1.5.3, rituximab, obinutuzumab, ibritumomab, ofatumumab, tositumumab, odronextamab, veltuzumab, ublituximab, and ocrelizumab. In any of these embodiments, the extracellular binding domain of the CD20 CAR can comprise or consist of the V_{H}, the V_{L}, and/or one or more CDRs of any of the antibodies.

In some embodiments, the extracellular binding domain of the CD20 CAR comprises an scFv derived from the Leu16 monoclonal antibody, which comprises the heavy chain variable region (V_{H}) and the light chain variable region (V_{L}) of Leu16 connected by a linker. See Wu et al., Protein Engineering. 14 (12): 1025-1033 (2001). In some embodiments, the linker is a 3×G₄S linker. In other embodiments, the linker is a Whitlow linker as described herein. In some embodiments, the amino acid sequences of different portions of the entire Leu16-derived scFv (also referred to as Leu16 scFv) and its different portions are provided in Table 14 below. In some embodiments, the CD20-specific scFv comprises or consists of an amino acid sequence set forth in SEQ ID NO:414, 415, or 419, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:414, 415, or 419 In some embodiments, the CD20-specific scFv may comprise one or more CDRs having amino acid sequences set forth in SEQ ID NOs: 416-418, 420, 421, and 422. In some embodiments, the CD20-specific scFv may comprise a light chain with one or more CDRs having amino acid sequences set forth in SEQ ID NOs: 416-418. In some embodiments, the CD20-specific scFv may comprise a heavy chain with one or more CDRs having amino acid sequences set forth in SEQ ID NOs: 420, 421, and 422. In any of these embodiments, the CD20-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD20 CAR comprises or consists of the one or more CDRs as described herein.

TABLE 14
Exemplary sequences of anti-CD20 scFv and components

| SEQ | | |
|---|---|---|
| ID NO: | Amino Acid Sequence | Description |
| 414 | DIVLTQSPAILSASPGEKVTMTCRASSS | Anti-CD20 Leu16 scFv |
| | VNYMDWYQKKPGSSPKPWIYATSNLA | entire sequence, with |
| | SGVPARFSGSGSGTSYSLTISRVEAEDA | Whitlow linker |
| | ATYYCQQWSFNPPTFGGGTKLEIKGSTS | |
| | GSGKPGSGEGSTKGEVQLQQSGAELVK | |
| | PGASVKMSCKASGYTFTSYNMHWVKQ | |
| | TPGQGLEWIGAIYPGNGDTSYNQKFKG | |
| | KATLTADKSSSTAYMQLSSLTSEDSAD | |
| | YYCARSNYYGSSYWFFDVWGAGTTVT | |
| | VSS | |
| 415 | DIVLTQSPAILSASPGEKVTMTCRASSS | Anti-CD20 Leu16 scFv |
| | VNYMDWYQKKPGSSPKPWIYATSNLA | light chain variable |
| | SGVPARFSGSGSGTSYSLTISRVEAEDA | region |
| | ATYYCQQWSFNPPTFGGGTKLEIK | |
| 416 | RASSSVNYMD | Anti-CD20 Leu16 scFv |
| | | light chain CDR1 |
| 417 | ATSNLAS | Anti-CD20 Leu16 scFv |
| | | light chain CDR2 |
| 418 | QQWSFNPPT | Anti-CD20 Leu16 scFv |
| | | light chain CDR3 |
| 419 | EVQLQQSGAELVKPGASVKMSCKASG | Anti-CD20 Leu16 scFv |
| | YTFTSYNMHWVKQTPGQGLEWIGAIYP | heavy chain |
| | GNGDTSYNQKFKGKATLTADKSSSTAY | |
| | MQLSSLTSEDSADYYCARSNYYGSSYW | |
| | FFDVWGAGTTVTVSS | |
| 420 | SYNMH | Anti-CD20 Leu16 scFv |
| | | heavy chain CDR1 |
| 421 | AIYPGNGDTSYNQKFKG | Anti-CD20 Leu16 scFv |
| | | heavy chain CDR2 |
| 422 | SNYYGSSYWFFDV | Anti-CD20 Leu16 scFv |
| | | heavy chain CDR3 |

In some embodiments, the hinge domain of the CD20 CAR comprises a CD8α hinge domain, for example, a human CD8α hinge domain. In some embodiments, the CD8α hinge domain comprises or consists of an amino acid sequence set forth in SEQ ID NO:378 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:378. In some embodiments, the hinge domain comprises a CD28 hinge domain, for example, a human CD28 hinge domain. In some embodiments, the CD28 hinge domain comprises or consists of an amino acid sequence set forth in SEQ ID NO:382 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:382. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO: 384 or SEQ ID NO:385, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:384 or SEQ ID NO:385. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:386 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:386.

In some embodiments, the transmembrane domain of the CD20 CAR comprises a CD8α transmembrane domain, for example, a human CD8α transmembrane domain. In some embodiments, the CD8α transmembrane domain comprises or consists of an amino acid sequence set forth in SEQ ID NO: 387 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:387. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:388 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:388.

In some embodiments, the intracellular costimulatory domain of the CD20 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:390 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:390. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:391 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:391.

In some embodiments, the intracellular signaling domain of the CD20 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:392 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:392.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:414, the CD8α hinge domain of SEQ ID NO: 381, the CD8α transmembrane domain of SEQ ID NO:387, the 4-1BB costimulatory domain of SEQ ID NO:390, the CD3ζ signaling domain of SEQ ID NO:392, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:414, the CD28 hinge domain of SEQ ID NO: 381, the CD8α transmembrane domain of SEQ ID NO:387, the 4-1BB costimulatory domain of SEQ ID NO:390, the CD3ζ signaling domain of SEQ ID NO:392, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:414, the IgG4 hinge domain of SEQ ID NO: 384 or SEQ ID NO:385, the CD8α transmembrane domain of SEQ ID NO:387, the 4-1BB costimulatory domain of SEQ ID NO:390, the CD3ζ signaling domain of SEQ ID NO:392, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:414, the CD8α hinge domain of SEQ ID NO: 387, the CD28 transmembrane domain of SEQ ID NO:389, the 4-1BB costimulatory domain of SEQ ID NO:390, the CD3ζ signaling domain of SEQ ID NO:392, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:414, the CD28 hinge domain of SEQ ID NO: 382, the CD28 transmembrane domain of SEQ ID NO:388, the 4-1BB costimulatory domain of SEQ ID NO:390, the CD3 signaling domain of SEQ ID NO:392, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:414, the IgG4 hinge domain of SEQ ID NO: 384 or SEQ ID NO:385, the CD28 transmembrane domain of SEQ ID NO:388, the 4-1BB costimulatory domain of SEQ ID NO:390, the CD3ζ signaling domain of SEQ ID NO:392, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

### 3) CD22 CAR

In some embodiments, the CAR is a CD22 CAR (“CD22-CAR”), and in these embodiments, the polycistronic vector comprises an expression cassette that contains a nucleotide sequence encoding a CD22 CAR. CD22, which is a transmembrane protein found mostly on the surface of mature B cells that functions as an inhibitory receptor for B cell receptor (BCR) signaling. CD22 is expressed in 60-70% of B cell lymphomas and leukemias (e.g., B-chronic lymphocytic leukemia, hairy cell leukemia, acute lymphocytic leukemia (ALL), and Burkitt's lymphoma) and is not present on the cell surface in early stages of B cell development or on stem cells. In some embodiments, the CD22 CAR may comprise a signal peptide, an extracellular binding domain that specifically binds CD22, a hinge domain, a transmembrane domain, an intracellular costimulatory domain, and/or an intracellular signaling domain in tandem.

In some embodiments, the signal peptide of the CD22 CAR comprises a CD8α signal peptide. In some embodiments, the CD8α signal peptide comprises or consists of an amino acid sequence set forth in SEQ ID NO:450 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:450. In some embodiments, the signal peptide comprises an IgK signal peptide. In some embodiments, the IgK signal peptide comprises or consists of an amino acid sequence set forth in SEQ ID NO:379 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 379. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:380 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:380.

In some embodiments, the extracellular binding domain of the CD22 CAR is specific to CD22, for example, human CD22. The extracellular binding domain of the CD22 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD22 CAR is derived from an antibody specific to CD22, including, for example, SM03, inotuzumab, epratuzumab, moxetumomab, and pinatuzumab. In any of these embodiments, the extracellular binding domain of the CD22 CAR can comprise or consist of the V_{H}, the V_{L}, and/or one or more CDRs of any of the antibodies.

In some embodiments, the extracellular binding domain of the CD22 CAR comprises an scFv derived from the m971 monoclonal antibody (m971), which comprises the heavy chain variable region (V_{H}) and the light chain variable region (V_{L}) of m971 connected by a linker. In some embodiments, the linker is a 3×G₄S linker. In other embodiments, the Whitlow linker may be used instead. In some embodiments, the amino acid sequences of the entire m971-derived scFv (also referred to as m971 scFv) and its different portions are provided in Table 15 below. In some embodiments, the CD22-specific scFv comprises or consists of an amino acid sequence set forth in SEQ ID NO:423 or 432, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:423 or 432. In some embodiments, the CD22-specific scFv may comprise one or more CDRs having amino acid sequences set forth in SEQ ID NOs: 425-427 and 429-431, and 434-436, and 438-440. In some embodiments, the CD22-specific scFv may comprise a heavy chain with one or more CDRs having amino acid sequences set forth in SEQ ID NOs: 425-427 or 434-436. In some embodiments, the CD22-specific scFv may comprise a light chain with one or more CDRs having amino acid sequences set forth in SEQ ID NOs: 429-431 or 438-440. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

In some embodiments, the extracellular binding domain of the CD22 CAR comprises an scFv derived from m971-L7, which is an affinity matured variant of m971 with significantly improved CD22 binding affinity compared to the parental antibody m971 (improved from about 2 nM to less than 50 μM). In some embodiments, the scFv derived from m971-L7 comprises the V_{H} and the V_{L} of m971-L7 connected by a 3×G₄S linker. In other embodiments, the Whitlow linker may be used instead. In some embodiments, the amino acid sequences of the entire m971-L7-derived scFv (also referred to as m971-L7 scFv) and its different portions are provided in Table 15 below. In some embodiments, the CD22-specific scFv comprises or consists of an amino acid sequence set forth in SEQ ID NO:423 or 432, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:423, or 432. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

TABLE 15
Exemplary sequences of anti-CD22 scFv and components

| SEQ | | |
|---|---|---|
| ID NO: | Amino Acid Sequence | Description |
| 423 | QVQLQQSGPGLVKPSQTLSLTCAISGDS | Anti-CD22 m971 scFv |
| | VSSNSAAWNWIRQSPSRGLEWLGRTYY | entire sequence, with |
| | RSKWYNDYAVSVKSRITINPDTSKNQFS | 3xG₄S linker |
| | LQLNSVTPEDTAVYYCAREVTGDLEDA | |
| | FDIWGQGTMVTVSSGGGGSGGGGSGG | |
| | GGSDIQMTQSPSSLSASVGDRVTITCRA | |
| | SQTIWSYLNWYQQRPGKAPNLLIYAAS | |
| | SLQSGVPSRFSGRGSGTDFTLTISSLQAE | |
| | DFATYYCQQSYSIPQTFGQGTKLEIK | |
| 424 | QVQLQQSGPGLVKPSQTLSLTCAISGDS | Anti-CD22 m971 scFv |
| | VSSNSAAWNWIRQSPSRGLEWLGRTYY | heavy chain variable |
| | RSKWYNDYAVSVKSRITINPDTSKNQFS | region |
| | LQLNSVTPEDTAVYYCAREVTGDLEDA | |
| | FDIWGQGTMVTVSS | |
| 425 | GDSVSSNSAA | Anti-CD22 m971 scFv |
| | | heavy chain CDR1 |
| 426 | TYYRSKWYN | Anti-CD22 m971 scFv |
| | | heavy chain CDR2 |
| 427 | AREVTGDLEDAFDI | Anti-CD22 m971 scFv |
| | | heavy chain CDR3 |
| 428 | DIQMTQSPSSLSASVGDRVTITCRASQTI | Anti-CD22 m971 scFv |
| | WSYLNWYQQRPGKAPNLLIYAASSLQS | light chain |
| | GVPSRFSGRGSGTDFTLTISSLQAEDFAT | |
| | YYCQQSYSIPQTFGQGTKLEIK | |
| 429 | QTIWSY | Anti-CD22 m971 scFv |
| | | light chain CDR1 |
| 430 | AAS | Anti-CD22 m971 scFv |
| | | light chain CDR2 |
| 431 | QQSYSIPQT | Anti-CD22 m971 scFv |
| | | light chain CDR3 |
| 432 | QVQLQQSGPGMVKPSQTLSLTCAISGD | Anti-CD22 m971-L7 scFv |
| | SVSSNSVAWNWIRQSPSRGLEWLGRTY | entire sequence, with |
| | YRSTWYNDYAVSMKSRITINPDTNKNQ | 3xG₄S linker |
| | FSLQLNSVTPEDTAVYYCAREVTGDLE | |
| | DAFDIWGQGTMVTVSSGGGGSGGGGS | |
| | GGGGSDIQMIQSPSSLSASVGDRVTITC | |
| | RASQTIWSYLNWYRQRPGEAPNLLIYA | |
| | ASSLQSGVPSRFSGRGSGTDFTLTISSLQ | |
| | AEDFATYYCQQSYSIPQTFGQGTKLEIK | |
| 433 | QVQLQQSGPGMVKPSQTLSLTCAISGD | Anti-CD22 m971-L7 scFv |
| | SVSSNSVAWNWIRQSPSRGLEWLGRTY | heavy chain variable |
| | YRSTWYNDYAVSMKSRITINPDTNKNQ | region |
| | FSLQLNSVTPEDTAVYYCAREVTGDLE | |
| | DAFDIWGQGTMVTVSS | |
| 434 | GDSVSSNSVA | Anti-CD22 m971-L7 scFv |
| | | heavy chain CDR1 |
| 435 | TYYRSTWYN | Anti-CD22 m971-L7 scFv |
| | | heavy chain CDR2 |
| 436 | AREVTGDLEDAFDI | Anti-CD22 m971-L7 scFv |
| | | heavy chain CDR3 |
| 437 | DIQMIQSPSSLSASVGDRVTITCRASQTI | Anti-CD22 m971-L7 scFv |
| | WSYLNWYRQRPGEAPNLLIYAASSLQS | light chain variable |
| | GVPSRFSGRGSGTDFTLTISSLQAEDFAT | region |
| | YYCQQSYSIPQTFGQGTKLEIK | |
| 438 | QTIWSY | Anti-CD22 m971-L7 scFv |
| | | light chain CDR1 |
| 439 | AAS | Anti-CD22 m971-L7 scFv |
| | | light chain CDR2 |
| 440 | QQSYSIPQT | Anti-CD22 m971-L7 scFv |
| | | light chain CDR3 |

In some embodiments, the extracellular binding domain of the CD22 CAR comprises immunotoxins HA22 or BL22. Immunotoxins BL22 and HA22 are therapeutic agents that comprise an scFv specific for CD22 fused to a bacterial toxin, and thus can bind to the surface of the cancer cells that express CD22 and kill the cancer cells. BL22 comprises a dsFv of an anti-CD22 antibody, RFB4, fused to a 38-kDa truncated form of Pseudomonas exotoxin A (Bang et al., Clin. Cancer Res., 11:1545-50 (2005)). HA22 (CAT8015, moxetumomab pasudotox) is a mutated, higher affinity version of BL22 (Ho et al., J. Biol. Chem., 280 (1): 607-17 (2005)). Suitable sequences of antigen binding domains of HA22 and BL22 specific to CD22 are disclosed in, for example, U.S. Pat. Nos. 7,541,034; 7,355,012; and 7,982,011, which are hereby incorporated by reference in their entirety.

In some embodiments, the hinge domain of the CD22 CAR comprises a CD8 hinge domain, for example, a human CD8α hinge domain. In some embodiments, the CD8α hinge domain comprises or consists of an amino acid sequence set forth in SEQ ID NO:381 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:381. In some embodiments, the hinge domain comprises a CD28 hinge domain, for example, a human CD28 hinge domain. In some embodiments, the CD28 hinge domain comprises or consists of an amino acid sequence set forth in SEQ ID NO:382 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:381. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO: 384 or SEQ ID NO:385, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:384 or SEQ ID NO:385. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:386 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:386.

In some embodiments, the transmembrane domain of the CD22 CAR comprises a CD8α transmembrane domain, for example, a human CD8α transmembrane domain. In some embodiments, the CD8α transmembrane domain comprises or consists of an amino acid sequence set forth in SEQ ID NO: 387 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:387. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:388 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:388.

In some embodiments, the intracellular costimulatory domain of the CD22 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:390 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:390. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:391 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:391.

In some embodiments, the intracellular signaling domain of the CD22 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:392 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:392.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:423 or SEQ ID NO:432, the CD8α hinge domain of SEQ ID NO:381, the CD8α transmembrane domain of SEQ ID NO:387, the 4-1BB costimulatory domain of SEQ ID NO:390, the CD3 signaling domain of SEQ ID NO:392, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:423 or SEQ ID NO:432, the CD28 hinge domain of SEQ ID NO:382, the CD8α transmembrane domain of SEQ ID NO:387, the 4-1BB costimulatory domain of SEQ ID NO:390, the CD3ζ signaling domain of SEQ ID NO:392, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:423 or SEQ ID NO:432, the IgG4 hinge domain of SEQ ID NO:384 or SEQ ID NO:385, the CD8α transmembrane domain of SEQ ID NO: 387, the 4-1BB costimulatory domain of SEQ ID NO:390, the CD3ζ signaling domain of SEQ ID NO: 392, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:423 or SEQ ID NO:432, the CD8α hinge domain of SEQ ID NO:381, the CD28 transmembrane domain of SEQ ID NO:389, the 4-1BB costimulatory domain of SEQ ID NO:390, the CD3ζ signaling domain of SEQ ID NO:391, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:423 or SEQ ID NO:432, the CD28 hinge domain of SEQ ID NO:383, the CD28 transmembrane domain of SEQ ID NO:389, the 4-1BB costimulatory domain of SEQ ID NO:390, the CD3ζ signaling domain of SEQ ID NO:392, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:423 or SEQ ID NO:432, the IgG4 hinge domain of SEQ ID NO:384 or SEQ ID NO:385, the CD28 transmembrane domain of SEQ ID NO: 388, the 4-1BB costimulatory domain of SEQ ID NO:390, the CD3ζ signaling domain of SEQ ID NO: 392, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

### 4) BCMA CAR

In some embodiments, the CAR is a BCMA CAR (“BCMA-CAR”), and in these embodiments, the polycistronic vector comprises an expression cassette that contains a nucleotide sequence encoding a BCMA CAR. BCMA is a tumor necrosis family receptor (TNFR) member expressed on cells of the B cell lineage, with the highest expression on terminally differentiated B cells or mature B lymphocytes. BCMA is involved in mediating the survival of plasma cells for maintaining long-term humoral immunity. The expression of BCMA has been recently linked to a number of cancers, such as multiple myeloma, Hodgkin's and non-Hodgkin's lymphoma, various leukemias, and glioblastoma. In some embodiments, the BCMA CAR may comprise a signal peptide, an extracellular binding domain that specifically binds BCMA, a hinge domain, a transmembrane domain, an intracellular costimulatory domain, and/or an intracellular signaling domain in tandem.

In some embodiments, the signal peptide of the BCMA CAR comprises a CD8α signal peptide. In some embodiments, the CD8α signal peptide comprises or consists of an amino acid sequence set forth in SEQ ID NO:378 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:378. In some embodiments, the signal peptide comprises an IgK signal peptide. In some embodiments, the IgK signal peptide comprises or consists of an amino acid sequence set forth in SEQ ID NO:379 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 379. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:380 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:380.

In some embodiments, the extracellular binding domain of the BCMA CAR is specific to BCMA, for example, human BCMA. The extracellular binding domain of the BCMA CAR can be codon-optimized for expression in a host cell or to have variant sequences to increase functions of the extracellular binding domain.

In some embodiments, the extracellular binding domain comprises an immunogenically active portion of an immunoglobulin molecule, for example, an scFv. In some embodiments, the extracellular binding domain of the BCMA CAR is derived from an antibody specific to BCMA, including, for example, belantamab, erlanatamab, teclistamab, LCAR-B38M, and ciltacabtagene. In any of these embodiments, the extracellular binding domain of the BCMA CAR can comprise or consist of the V_{H}, the V_{L}, and/or one or more CDRs of any of the antibodies.

In some embodiments, the extracellular binding domain of the BCMA CAR comprises an scFv derived from C11D5.3, a murine monoclonal antibody as described in Carpenter et al., Clin. Cancer Res. 19 (8): 2048-2060 (2013). See also PCT Application Publication No. WO2010/104949. The C11D5.3-derived scFv may comprise the heavy chain variable region (V_{H}) and the light chain variable region (V_{L}) of C11D5.3 connected by the Whitlow linker, the amino acid sequences of which is provided in Table 17 below. In some embodiments, the BCMA-specific extracellular binding domain comprises or consists of an amino acid sequence set forth in SEQ ID NO:441, 450, or 463, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:441, 450, or 463. In some embodiments, the BCMA-specific extracellular binding domain may comprise one or more CDRs having amino acid sequences set forth in SEQ ID NOs: 443-445, 447-449, 452-454, 456-458, 460-432, 465-467 and 469-471. In some embodiments, the BCMA-specific extracellular binding domain may comprise a light chain with one or more CDRs having amino acid sequences set forth in SEQ ID NOs: 443-445, 452-454, 465-467. In some embodiments, the BCMA-specific extracellular binding domain may comprise a heavy chain with one or more CDRs having amino acid sequences set forth in SEQ ID NOs: 447-449, 456-458, 469-471. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from another murine monoclonal antibody, C12A3.2, as described in Carpenter et al., Clin. Cancer Res. 19 (8): 2048-2060 (2013) and PCT Application Publication No. WO2010/104949, the amino acid sequence of which is also provided in Table 16 below. In some embodiments, the BCMA-specific extracellular binding domain comprises or consists of an amino acid sequence set forth in SEQ ID NO: 441, 450 459, or 463, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 441, 450 459, or 463. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises a murine monoclonal antibody with high specificity to human BCMA, referred to as BB2121 in Friedman et al., Hum. Gene Ther. 29 (5): 585-601 (2018)). See also, PCT Application Publication No. WO2012163805.

In some embodiments, the extracellular binding domain of the BCMA CAR comprises single variable fragments of two heavy chains (VHH) that can bind to two epitopes of BCMA as described in Zhao et al., J. Hematol. Oncol. 11 (1): 141 (2018), also referred to as LCAR-B38M. See also, PCT Application Publication No. WO2018/028647.

In some embodiments, the extracellular binding domain of the BCMA CAR comprises a fully human heavy-chain variable domain (FHVH) as described in Lam et al., Nat. Commun. 11 (1): 283 (2020), also referred to as FHVH33. See also, PCT Application Publication No. WO2019/006072. The amino acid sequences of FHVH33 and its CDRs are provided in Table 16 below. In some embodiments, the BCMA-specific extracellular binding domain comprises or consists of an amino acid sequence set forth in SEQ ID NO:164 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:164. In some embodiments, the BCMA-specific extracellular binding domain may comprise one or more CDRs having amino acid sequences set forth in SEQ ID NOs: 165-167. In any of these embodiments, the BCMA-specific extracellular binding domain may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from CT103A (or CAR0085) as described in U.S. Pat. No. 11,026,975 B2, the amino acid sequence of which is provided in Table 16 below. In some embodiments, the BCMA-specific extracellular binding domain comprises or consists of an amino acid sequence set forth in SEQ ID NO: 463, 464, or 468, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 463, 464, or 468. In some embodiments, the BCMA-specific extracellular binding domain may comprise one or more CDRs having amino acid sequences set forth in SEQ ID NOs: 465-497 and 469-471. In some embodiments, the BCMA-specific extracellular binding domain may comprise a light chain with one or more CDRs having amino acid sequences set forth in SEQ ID NOs: 465-497. In some embodiments, the BCMA-specific extracellular binding domain may comprise a heavy chain with one or more CDRs having amino acid sequences set forth in SEQ ID NOs: 469-471. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

Additionally, CARs and binders directed to BCMA have been described in U.S. Application Publication Nos. 2020/0246381 A1 and 2020/0339699 A1, the entire contents of each of which are incorporated by reference herein.

TABLE 16
Exemplary sequences of anti-BCMA binder and components

| SEQ | | |
|---|---|---|
| ID NO: | Amino Acid Sequence | Description |
| 441 | DIVLTQSPASLAMSLGKRATISCRASES | Anti-BCMA C11D5.3 |
| | VSVIGAHLIHWYQQKPGQPPKLLIYLAS | scFv entire sequence, |
| | NLETGVPARFSGSGSGTDFTLTIDPVEE | with Whitlow linker |
| | DDVAIYSCLQSRIFPRTFGGGTKLEIKGS | |
| | TSGSGKPGSGEGSTKGQIQLVQSGPELK | |
| | KPGETVKISCKASGYTFTDYSINWVKR | |
| | APGKGLKWMGWINTETREPAYAYDFR | |
| | GRFAFSLETSASTAYLQINNLKYEDTAT | |
| | YFCALDYSYAMDYWGQGTSVTVSS | |
| 442 | DIVLTQSPASLAMSLGKRATISCRASES | Anti-BCMA C11D5.3 |
| | VSVIGAHLIHWYQQKPGQPPKLLIYLAS | scFv light chain |
| | NLETGVPARFSGSGSGTDFTLTIDPVEE | variable region |
| | DDVAIYSCLQSRIFPRTFGGGTKLEIK | |
| 443 | RASESVSVIGAHLIH | Anti-BCMA C11D5.3 |
| | | scFv light chain CDR1 |
| 444 | LASNLET | Anti-BCMA C11D5.3 |
| | | scFv light chain CDR2 |
| 445 | LQSRIFPRT | Anti-BCMA C11D5.3 |
| | | scFv light chain CDR3 |
| 446 | QIQLVQSGPELKKPGETVKISCKASGYT | Anti-BCMA C11D5.3 |
| | FTDYSINWVKRAPGKGLKWMGWINTE | scFv heavy chain |
| | TREPAYAYDFRGRFAFSLETSASTAYLQ | variable region |
| | INNLKYEDTATYFCALDYSYAMDYWG | |
| | QGTSVTVSS | |
| 447 | DYSIN | Anti-BCMA C11D5.3 |
| | | scFv heavy chain CDR1 |
| 448 | WINTETREPAYAYDFRG | Anti-BCMA C11D5.3 |
| | | scFv heavy chain CDR2 |
| 449 | DYSYAMDY | Anti-BCMA C11D5.3 |
| | | scFv heavy chain CDR3 |
| 450 | DIVLTQSPPSLAMSLGKRATISCRASESV | Anti-BCMA C12A3.2 |
| | TILGSHLIYWYQQKPGQPPTLLIQLASN | scFv entire sequence, |
| | VQTGVPARFSGSGSRTDFTLTIDPVEED | with Whitlow linker |
| | DVAVYYCLQSRTIPRTFGGGTKLEIKGS | |
| | TSGSGKPGSGEGSTKGQIQLVQSGPELK | |
| | KPGETVKISCKASGYTFRHYSMNWVK | |
| | QAPGKGLKWMGRINTESGVPIYADDFK | |
| | GRFAFSVETSASTAYLVINNLKDEDTAS | |
| | YFCSNDYLYSLDFWGQGTALTVSS | |
| 451 | DIVLTQSPPSLAMSLGKRATISCRASESV | Anti-BCMA C12A3.2 |
| | TILGSHLIYWYQQKPGQPPTLLIQLASN | scFv light chain |
| | VQTGVPARFSGSGSRTDFTLTIDPVEED | variable region |
| | DVAVYYCLQSRTIPRTFGGGTKLEIK | |
| 452 | RASESVTILGSHLIY | Anti-BCMA C12A3.2 |
| | | scFv light chain CDR1 |
| 453 | LASNVQT | Anti-BCMA C12A3.2 |
| | | scFv light chain CDR2 |
| 454 | LQSRTIPRT | Anti-BCMA C12A3.2 |
| | | scFv light chain CDR3 |
| 455 | QIQLVQSGPELKKPGETVKISCKASGYT | Anti-BCMA C12A3.2 |
| | FRHYSMNWVKQAPGKGLKWMGRINTE | scFv heavy chain |
| | SGVPIYADDFKGRFAFSVETSASTAYLV | variable region |
| | INNLKDEDTASYFCSNDYLYSLDFWGQ | |
| | GTALTVSS | |
| 456 | HYSMN | Anti-BCMA C12A3.2 |
| | | scFv heavy chain CDR1 |
| 457 | RINTESGVPIYADDFKG | Anti-BCMA C12A3.2 |
| | | scFv heavy chain CDR2 |
| 458 | DYLYSLDF | Anti-BCMA C12A3.2 |
| | | scFv heavy chain CDR3 |
| 459 | EVQLLESGGGLVQPGGSLRLSCAASGF | Anti-BCMA FHVH33 |
| | TFSSYAMSWVRQAPGKGLEWVSSISGS | entire sequence |
| | GDYIYYADSVKGRFTISRDISKNTLYLQ | |
| | MNSLRAEDTAVYYCAKEGTGANSSLA | |
| | DYRGQGTLVTVSS | |
| 460 | GFTFSSYA | Anti-BCMA FHVH33 |
| | | CDR1 |
| 461 | ISGSGDYI | Anti-BCMA FHVH33 |
| | | CDR2 |
| 462 | AKEGTGANSSLADY | Anti-BCMA FHVH33 |
| | | CDR3 |
| 463 | DIQMTQSPSSLSASVGDRVTITCRASQSI | Anti-BCMA CT103A |
| | SSYLNWYQQKPGKAPKLLIYAASSLQS | scFv entire sequence, |
| | GVPSRFSGSGSGTDFTLTISSLQPEDFAT | with Whitlow linker |
| | YYCQQKYDLLTFGGGTKVEIKGSTSGS | |
| | GKPGSGEGSTKGQLQLQESGPGLVKPS | |
| | ETLSLTCTVSGGSISSSSYYWGWIRQPP | |
| | GKGLEWIGSISYSGSTYYNPSLKSRVTIS | |
| | VDTSKNQFSLKLSSVTAADTAVYYCAR | |
| | DRGDTILDVWGQGTMVTVSS | |
| 464 | DIQMTQSPSSLSASVGDRVTITCRASQSI | Anti-BCMA CT103A |
| | SSYLNWYQQKPGKAPKLLIYAASSLQS | scFv light chain |
| | GVPSRFSGSGSGTDFTLTISSLQPEDFAT | variable region |
| | YYCQQKYDLLTFGGGTKVEIK | |
| 465 | QSISSY | Anti-BCMA CT103A |
| | | scFv light chain CDR1 |
| 466 | AAS | Anti-BCMA CT103A |
| | | scFv light chain CDR2 |
| 467 | QQKYDLLT | Anti-BCMA CT103A |
| | | scFv light chain CDR3 |
| 468 | QLQLQESGPGLVKPSETLSLTCTVSGGS | Anti-BCMA CT103A |
| | ISSSSYYWGWIRQPPGKGLEWIGSISYS | scFv heavy chain |
| | GSTYYNPSLKSRVTISVDTSKNQFSLKL | variable region |
| | SSVTAADTAVYYCARDRGDTILDVWG | |
| | QGTMVTVSS | |
| 469 | GGSISSSSYY | Anti-BCMA CT103A |
| | | scFv heavy chain CDR1 |
| 470 | ISYSGST | Anti-BCMA CT103A |
| | | scFv heavy chain CDR2 |
| 471 | ARDRGDTILDV | Anti-BCMA CT103A |
| | | scFv heavy chain CDR3 |

In some embodiments, the hinge domain of the BCMA CAR comprises a CD8α hinge domain, for example, a human CD8α hinge domain. In some embodiments, the CD8α hinge domain comprises or consists of an amino acid sequence set forth in SEQ ID NO:381 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 381. In some embodiments, the hinge domain comprises a CD28 hinge domain, for example, a human CD28 hinge domain. In some embodiments, the CD28 hinge domain comprises or consists of an amino acid sequence set forth in SEQ ID NO:382 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:382. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:384 or SEQ ID NO:385, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:384 or SEQ ID NO: 385. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:386 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:386.

In some embodiments, the transmembrane domain of the BCMA CAR comprises a CD8α transmembrane domain, for example, a human CD8α transmembrane domain. In some embodiments, the CD8α transmembrane domain comprises or consists of an amino acid sequence set forth in SEQ ID NO: 387 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:387. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:388 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:388.

In some embodiments, the intracellular costimulatory domain of the BCMA CAR comprises a 4-1BB costimulatory domain, for example, a human 4-1BB costimulatory domain. In some embodiments, the 4-1BB costimulatory domain comprises or consists of an amino acid sequence set forth in SEQ ID NO:390 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:390. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:391 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:391.

In some embodiments, the intracellular signaling domain of the BCMA CAR comprises a CD3 zeta (ζ) signaling domain, for example, a human CD3ζ signaling domain. In some embodiments, the CD3ζ signaling domain comprises or consists of an amino acid sequence set forth in SEQ ID NO:392 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:392.

In some embodiments, the polycistronic vector comprises an expression cassette that contains a nucleotide sequence encoding a BCMA CAR, including, for example, a BCMA CAR comprising any of the BCMA-specific extracellular binding domains as described, the CD8a hinge domain of SEQ ID NO:381, the CD8a transmembrane domain of SEQ ID NO:387, the 4-1BB costimulatory domain of SEQ ID NO:390, the CD35 signaling domain of SEQ ID NO:392, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide (e.g., a CD8a signal peptide) as described.

In some embodiments, the polycistronic vector comprises an expression cassette that contains a nucleotide sequence encoding a BCMA CAR, including, for example, a BCMA CAR comprising any of the BCMA-specific extracellular binding domains as described, the CD8a hinge domain of SEQ ID NO:381, the CD8a transmembrane domain of SEQ ID NO:387, the CD28 costimulatory domain of SEQ ID NO:391, the CD32 signaling domain of SEQ ID NO:392, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide as described.

In some embodiments, the polycistronic vector comprises an expression cassette that contains a nucleotide sequence encoding a BCMA CAR as set forth in SEQ ID NO:406 or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:406 (see Table 17). The encoded BCMA CAR has a corresponding amino acid sequence set forth in SEQ ID NO:407 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 407, with the following components: CD8a signal peptide, CT103A scFv (VL-Whitlow linker-VH), CD8a hinge domain, CD8a transmembrane domain, 4-1BB costimulatory domain, and CD32 signaling domain.

In some embodiments, the polycistronic vector comprises an expression cassette that contains a nucleotide sequence encoding a commercially available embodiment of BCMA CAR, including, for example, idecabtagene vicleucel (ide-cel, also called bb2121). In some embodiments, the polycistronic vector comprises an expression cassette that contains a nucleotide sequence encoding idecabtagene vicleucel or portions thereof. Idecabtagene vicleucel comprises a BCMA CAR with the following components: the BB2121 binder, CD8a hinge domain, CD8a transmembrane domain, 4-1BB costimulatory domain, and CD3ζ signaling domain.

TABLE 17
Exemplary sequences of BCMA CARs

| SEQ | | |
|---|---|---|
| ID NO: | Sequence | Description |
| 472 | atggccttaccagtgaccgccttgctcctgccgctggccttgctgctcca | Exemplary BCMA |
| | cgccgccaggccggacatccagatgacccagtctccatcctccctgtct | CAR nucleotide |
| | gcatctgtaggagacagagtcaccatcacttgccgggcaagtcagagc | sequence |
| | attagcagctatttaaattggtatcagcagaaaccagggaaagcccctaa | |
| | gctcctgatctatgctgcatccagtttgcaaagtggggtcccatcaaggtt | |
| | cagtggcagtggatctgggacagatttcactctcaccatcagcagtctgc | |
| | aacctgaagattttgcaacttactactgtcagcaaaaatacgacctcctca | |
| | cttttggcggagggaccaaggttgagatcaaaggcagcaccagcggct | |
| | ccggcaagcctggctctggcgagggcagcacaaagggacagctgca | |
| | gctgcaggagtcgggcccaggactggtgaagccttcggagaccctgtc | |
| | cctcacctgcactgtctctggtggctccatcagcagtagtagttactactg | |
| | gggctggatccgccagcccccagggaaggggctggagtggattggg | |
| | agtatctcctatagtgggagcacctactacaacccgtccctcaagagtcg | |
| | agtcaccatatccgtagacacgtccaagaaccagttctccctgaagctga | |
| | gttctgtgaccgccgcagacacggcggtgtactactgcgccagagatc | |
| | gtggagacaccatactagacgtatggggtcagggtacaatggtcaccgt | |
| | cagctcattcgtgcccgtgttcctgcccgccaaacctaccaccacccctg | |
| | cccctagacctcccaccccagccccaacaatcgccagccagcctctgt | |
| | ctctgcggcccgaagcctgtagacctgctgccggcggagccgtgcaca | |
| | ccagaggcctggacttcgcctgcgacatctacatctgggcccctctggc | |
| | cggcacctgtggcgtgctgctgctgagcctggtgatcaccctgtactgc | |
| | aaccaccggaacaaacggggcagaaagaaactcctgtatatattcaaa | |
| | caaccatttatgagaccagtacaaactactcaagaggaagatggctgta | |
| | gctgccgatttccagaagaagaagaaggaggatgtgaactgagagtga | |
| | agttcagcagatccgccgacgcccctgcctaccagcagggacagaac | |
| | cagctgtacaacgagctgaacctgggcagacgggaagagtacgacgt | |
| | gctggacaagcggagaggccgggaccccgagatgggcggaaagcc | |
| | cagacggaagaacccccaggaaggcctgtataacgaactgcagaaag | |
| | acaagatggccgaggcctacagcgagatcggcatgaagggcgagcg | |
| | gaggcgcggcaagggccacgatggcctgtaccagggcctgagcacc | |
| | gccaccaaggacacctacgacgccctgcacatgcaggccctgccccc | |
| | caga | |
| 473 | MALPVTALLLPLALLLHAARPDIQMTQSPSSL | Exemplary BCMA |
| | SASVGDRVTITCRASQSISSYLNWYQQKPGKA | CAR amino acid |
| | PKLLIYAASSLQSGVPSRFSGSGSGTDFTLTISS | sequence |
| | LQPEDFATYYCQQKYDLLTFGGGTKVEIKGST | |
| | SGSGKPGSGEGSTKGQLQLQESGPGLVKPSET | |
| | LSLTCTVSGGSISSSSYYWGWIRQPPGKGLEWI | |
| | GSISYSGSTYYNPSLKSRVTISVDTSKNQFSLK | |
| | LSSVTAADTAVYYCARDRGDTILDVWGQGT | |
| | MVTVSSFVPVFLPAKPTTTPAPRPPTPAPTIAS | |
| | QPLSLRPEACRPAAGGAVHTRGLDFACDIYIW | |
| | APLAGTCGVLLLSLVITLYCNHRNKRGRKKLL | |
| | YIFKQPFMRPVQTTQEEDGCSCRFPEEEEGGC | |
| | ELRVKFSRSADAPAYQQGQNQLYNELNLGRR | |
| | EEYDVLDKRRGRDPEMGGKPRRKNPQEGLY | |
| | NELQKDKMAEAYSEIGMKGERRRGKGHDGL | |
| | YQGLSTATKDTYDALHMQALPPR | |

### 3. Gene Editing Enzymes

In some embodiments, the exogenous agent is or comprises a genome editing technology. In some embodiments, the exogenous agent is or comprises a heterologous protein that is associated with a genome editing technology. Any of a variety of agents associated with gene editing technologies can be included as the exogenous agent and/or heterologous protein, such as for delivery of gene editing machinery to a cell. In some embodiments, the gene editing technology can include systems involving nuclease, nickase, homing, integrase, transposase, recombinase, and/or reverse transcriptase activity. In some embodiments, the gene editing technologies can be used for knock-out or knock-down of genes. In some embodiments, the gene-editing technologies can be used for knock-in or integration of DNA into a region of the genome. In some embodiments, the exogenous agent and/or heterologous protein mediates single-strand breaks (SSB). In some embodiments, the exogenous agent and/or heterologous protein mediates double-strand breaks (DSB), including in connection with non-homologous end-joining (NHEJ) or homology-directed repair (HDR). In some embodiments, the exogenous agent and/or heterologous protein does not mediate SSB. In some embodiments, the exogenous agent and/or heterologous protein does not mediate DSB. In some embodiments, the exogenous agent and/or heterologous protein can be used for DNA base editing or prime-editing. In some embodiments, the exogenous agent and/or heterologous protein can be used for Programmable Addition via Site-specific Targeting Elements (PASTE). In some embodiments, the payload agent is a programmable DNA-binding polypeptide.

In some embodiments, the exogenous agent is a programmable DNA-binding polypeptide and/or nuclease for use in gene editing methods. In some embodiments, the nuclease is a zinc-finger nucleases (ZFNs), transcription-activator like effector nucleases (TALENs), or a CRISPR-associated protein-nuclease (Cas). In some embodiments, the programmable DNA-binding polypeptide is a CRISPR-associated protein-nuclease (Cas). In some embodiments, the Cas protein is selected from the group consisting of Cas3, Cas9, Cas10, Cas12, and Cas13. In some embodiments, the Cas is a Cas12a (also known as cpf1) from a Prevotella, Francisella novicida, Acidaminococcus sp., Lachnospiraceae bacterium, or Francisella bacteria. In some embodiments, the Cas is Cas9 from Streptococcus pyogenes. In some embodiments, the Cas is Cas9 from Streptococcus pyogenes (SpCas). In some embodiments, the Cas9 is from Staphylococcus aureus (SaCas9). In some embodiments, the Cas9 is from Neisseria meningitidis (NmeCas9). In some embodiments, the Cas9 is from Campylobacter jejuni (CjCas9). In some embodiments, the Cas9 is from Streptococcus thermophilis (StCas9). In some embodiments, the Cas is a Cas12a (also known as Cpf1) from a Prevotella or Francisella bacteria, or the Cas is a Cas12b from a Bacillus, optionally Bacillus hisashii. In some embodiments, the Cas is a Cas12a (also known as cpf1) from a Prevotella, Francisella novicida, Acidaminococcus sp., Lachnospiraceae bacterium, or Francisella bacteria. In some embodiments, the nuclease is MAD7 or CasX. In some of any embodiments, the Cas is a Cas3, Cas13, CasMini, or any other Cas protein known in the art. See for example, Wang et al., Biosensors and Bioelectronics (165) 1:2020, and Wu et al. Nature Reviews Chemistry (4) 441:2020). The Cas9 nuclease can, in some embodiments, be a Cas9 or functional fragment thereof from any bacterial species. See, e.g., Makarova et al. Nature Reviews, Microbiology, 9:467-477 (2011), including supplemental information, hereby incorporated by reference in its entirety.

In some embodiments, delivery of the nuclease is by a provided vector encoding the programmable DNA-binding polypeptide and/or the nuclease (e.g. Cas). In some embodiments, delivery of the programmable DNA-binding polypeptide and/or the nuclease is by a provided vector comprising an RNA encoding the programmable DNA-binding polypeptide and/or nuclease (e.g. Cas, Cas nickase, catalytically inactive Cas). In some embodiments, delivery of the programmable DNA-binding polypeptide and/or nuclease is by a provided VLP comprising an RNA encoding the programmable DNA-binding polypeptide and/or nuclease (e.g. Cas, Cas nickase, catalytically inactive Cas). In some embodiments, the Cas is a catalytically active Cas. In some embodiments, the Cas is a catalytically inactive Cas (also known as a dead Cas, dCas) that is a Cas that contains one or more mutations that inactivates the catalytic activity of the domain. In some embodiments, the Cas is a Cas nickase.

In some embodiments, the provided viral vector particles contain a nuclease protein and the programmable DNA-binding polypeptide and/or nuclease protein is directly delivered to a target cell. Methods of delivering a programmable DNA-binding polypeptide and/or nuclease protein include those as described, for example, in Cai et al. Elife, 2014, 3: e01911 and International patent publication No. WO2017068077. For instance, provided viral vector particles comprise one or more Cas protein(s), such as Cas9. In some embodiments, the programmable DNA-binding polypeptide and/or nuclease protein (e.g. Cas, such as Cas 9) is engineered as a chimeric nuclease protein with a viral structural protein (e.g. GAG) for packaging into the viral vector particle (e.g. lentiviral vector particle). For instance, a chimeric Cas9-protein fusion with the structural GAG protein can be packaged inside a lentiviral vector particle. In some embodiments, the fusion protein is a cleavable fusion protein between (i) a viral structural protein (e.g. GAG) and (ii) a nuclease protein (e.g. Cas protein, such as Cas 9).

In some cases, the cleavable fusion protein comprising a viral structural protein (e.g. a GAG protein (e.g. an MLV-gag or an HIV-gag)) and (ii) a programmable DNA-binding protein and/or nuclease protein (e.g. Cas protein (e.g. any of the Cas proteins described herein)) includes a nuclear export sequence (NES). In some embodiments, the NES facilitates localization of the fusion protein to the cytosol.

In some embodiments, the cleavable fusion protein includes at least one NES sequences (e.g. 2 or more, 3 or more, 4 or more, or 5 or more NES sequences). In some embodiments, one or more NES sequences (2 or more, 3 or more, 4 or more, or 5 or more NES sequences) are positioned at or near (e.g., within 50 amino acids of) the N-terminus and/or the C-terminus of the cleavable fusion protein. In one embodiment, the cleavable fusion protein includes an NES sequence is positioned at the N-terminus and an NES sequence is positioned at the C-terminus of the fusion protein.

In some embodiments, the cleavable fusion protein includes at least one NES sequences (e.g. 2 or more, 3 or more, 4 or more, or 5 or more NES sequences) positioned at or near (e.g. within 50 amino acids of) the N-terminus and/or the C-terminus of the programmable DNA-binding protein and/or nuclease protein. In one embodiment, the cleavable fusion protein includes an NES sequence is positioned at the N-terminus and/or an NES sequence is positioned at the C-terminus of the programmable DNA-binding protein and/or nuclease protein.

In some cases, the cleavable fusion protein comprising a viral structural protein (e.g. a GAG protein (e.g. an MLV-gag or an HIV-gag)) and (ii) a programmable DNA-binding protein and/or nuclease protein (e.g. Cas protein (e.g. any of the Cas proteins described herein)) includes a nuclear localization signal (NLS). In some embodiments, the NLS facilitates delivery of the fusion protein, or a therapeutic polypeptide (or polynucleotide encoding the therapeutic polypeptide) released from the fusion protein (for instance, a polypeptide (or polynucleotide encoding a polypeptide) released from the fusion protein following cleavage of the cleavable linker), into the nucleus of a target cell.

In some embodiments, the cleavable fusion protein includes at least one NLS sequences (e.g. 2 or more, 3 or more, 4 or more, or 5 or more NLS sequences). In some embodiments, one or more NLS sequences (2 or more, 3 or more, 4 or more, or 5 or more NLS sequences) are positioned at or near (e.g., within 50 amino acids of) the N-terminus and/or the C-terminus of the cleavable fusion protein. In one embodiment, the cleavable fusion protein includes an NLS sequence is positioned at the N-terminus and an NLS sequence is positioned at the C-terminus of the fusion protein.

In some embodiments, the cleavable fusion protein includes at least one NLS sequences (e.g. 2 or more, 3 or more, 4 or more, or 5 or more NLS sequences) positioned at or near (e.g., within 50 amino acids of) the N-terminus and/or the C-terminus of the programmable DNA-binding protein and/or nuclease protein. In one embodiment, the cleavable fusion protein includes an NLS sequence is positioned at the N-terminus and/or an NLS sequence is positioned at the C-terminus of the nuclease protein.

In some embodiments, the cleavable fusion protein comprises one NES sequence and one NLS sequences. In some embodiments, the viral structural protein, the NES sequence, NLS sequence, and the therapeutic polypeptide (e.g. the programmable DNA-binding protein and/or nuclease protein) sequence are positioned from N-terminus to C-terminus as follows: viral structural protein-NES-NLS-therapeutic polypeptide or viral structural protein-NES-therapeutic polypeptide-NLS. In some embodiments, the viral structural protein, the NES sequences, NLS sequence, and the therapeutic polypeptide sequence are positioned from N-terminus to C-terminus as follows: viral structural protein-NESⁿ-NLS-therapeutic polypeptide or viral structural protein-NESⁿ-therapeutic polypeptide-NLS, where n is equal to or greater than 2. In such embodiments, the cleavable linker is located before the therapeutic polypeptide (e.g., before an NLS that immediately precedes the therapeutic polypeptide).

In some embodiments, the cleavable fusion protein comprises one NES sequence and two NLS sequences. In some embodiments, the viral structural protein, the NES sequence, NLS sequences, and the therapeutic polypeptide (e.g. the programmable DNA-binding protein and/or nuclease protein) sequences are positioned from N-terminus to C-terminus as follows: viral structural protein-NES-NLS-therapeutic polypeptide-NLS. In some embodiments, the viral structural protein, the NES sequences, NLS sequences, and the therapeutic polypeptide sequence are positioned from N-terminus to C-terminus as follows: viral structural protein-NESⁿ-NLS-therapeutic polypeptide-NLS, where n is equal to or greater than 2. In such embodiments, the cleavable linker is located before the therapeutic polypeptide (e.g., before an NLS that immediately precedes the therapeutic polypeptide). In one embodiment, the cleavable fusion protein comprises from N-terminus to C-terminus: viral structural protein-NES-cleavable linker-NLS-therapeutic polypeptide-NLS.

In some embodiments, the cleavable fusion protein has a configuration selected from: gag-cleavage site-NLS-therapeutic polypeptide (e.g. a Cas protein)-NLS; gag-NES (3×)-cleavage site-NLS-therapeutic polypeptide (e.g. a Cas protein)-NLS; and gag-cleavage site-NLS-therapeutic polypeptide (e.g. a Cas protein)-NLS-cleavage site-NES.

In some embodiments, the Cas is wild-type Cas9, which can site-specifically cleave double-stranded DNA, resulting in the activation of the double-strand break (DSB) repair machinery. DSBs can be repaired by the cellular Non-Homologous End Joining (NHEJ) pathway (Overballe-Petersen et al., 2013, Proc Natl Acad Sci USA, Vol. 110:19860-19865), resulting in insertions and/or deletions (indels) which disrupt the targeted locus. Alternatively, if a donor template with homology to the targeted locus is supplied, the DSB may be repaired by the homology-directed repair (HDR) pathway allowing for precise replacement mutations to be made (Overballe-Petersen et al., 2013, Proc Natl Acad Sci USA, Vol. 110:19860-19865; Gong et al., 2005, Nat. Struct Mol Biol, Vol. 12:304-312). In some embodiments, the Cas is mutant form, known as Cas9 D10A, with only nickase activity. This means that Cas9D10A cleaves only one DNA strand, and does not activate NHEJ. Instead, when provided with a homologous repair template, DNA repairs are conducted via the high-fidelity HDR pathway only, resulting in reduced indel mutations (Cong et al., 2013, Science, Vol. 339:819-823; Jinek et al., 2012, Science, Vol. 337:816-821; Qi et al., 2013 Cell, Vol. 152:1173-1183). Cas9D10A is even more appealing in terms of target specificity when loci are targeted by paired Cas9 complexes designed to generate adjacent DNA nicks (Ran et al., 2013, Cell, Vol. 154:1380-1389). In some embodiments, the Cas is a nuclease-deficient Cas9 (Qi et al., 2013 Cell, Vol. 152:1173-1183). For instance, mutations H840A in the HNH domain and D10A in the RuvC domain inactivate cleavage activity, but do not prevent DNA binding. Therefore, this variant can be used to target in a sequence-specific manner any region of the genome without cleavage. Instead, by fusing with various effector domains, dCas9 can be used either as a gene silencing or activation tools. Furthermore, it can be used as a visualization tool by coupling the guide RNA or the Cas9 protein to a fluorophore or a fluorescent protein.

In some embodiments, the Cas protein comprises one or more mutations such that the Cas protein is converted into a nickase that is able to cleave only one strand of a double stranded DNA molecule (e.g., a SSB). For example, Cas9, which is normally capable of inducing a double strand break, can be converted into a Cas9 nickase, which is capable of inducing a single strand break, by mutating one of two Cas9 catalytic domains: the RuvC domain, which comprises the RuvC I, RuvC II, and RuvC III motifs, or the NHN domain. In some embodiments, the Cas protein comprises one or more mutations in the RuvC catalytic domain or the HNH catalytic domain. In some embodiments, the genome-modifying protein is a recombinant nuclease that has been modified to have nickase activity. In some embodiments, the recombinant nuclease cleaves the strand to which the guide RNA, e.g., sgRNA, hybridizes, but does not cleave the strand that is complementary to the strand to which the guide RNA, e.g., sgRNA, hybridizes. In some embodiments, the recombinant nuclease does not cleave the strand to which the guide RNA, e.g., sgRNA, hybridizes, but does cleave the strand that is complementary to the strand to which the guide RNA, e.g., sgRNA, hybridizes.

In some embodiments, the Cas protein is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7. In some embodiments, the Cas protein is Cas9. In some embodiments, the Cas9 is from a bacteria selected from the group consisting of Streptococcus pyogenes, Staphylococcus aureus, Neisseria meningitides, Campylobacter jejuni, and Streptococcus thermophilis. In some embodiments, the Cas9 is from Streptococcus pyogenes. In some embodiments, the Cas9 is from Streptococcus pyogenes and comprises one or more mutations in the RuvC I, RuvC II, or RuvC III motifs. In some embodiments, the Cas9 is from Streptococcus pyogenes and comprises a D10A mutation in the RuvC I motif. In some embodiments, the Cas9 is from Streptococcus pyogenes and comprises one or more mutations in the HNH catalytic domain. In some embodiments, the Cas9 is from Streptococcus pyogenes and comprises one or more mutations in the HNH catalytic domain selected from the group consisting of H840A, H854A, and H863A. In some embodiments, the Cas9 is from Streptococcus pyogenes and comprises a H840A mutation in the HNH catalytic domain. In some embodiments, the Cas9 is from Streptococcus pyogenes and comprises a mutation selected from the group consisting of D10A, H840A, H854A, and H863A.

In some embodiments, the Cas protein is selected from the group consisting of Cas3, Cas9, Cas10, Cas12, and Cas13. In particular embodiments, the nuclease is a Cas nuclease, such as Cas9. In some embodiments, delivery of the CRISPR/Cas can be used to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. In some embodiments, the one or more agent(s) (e.g., the heterologous protein) capable of inducing a DSB comprise Cas9 or a functional fragment thereof, and a first guide RNA, e.g., a first sgRNA, and a second guide RNA, e.g., a second sgRNA. The guide RNA, e.g., the first guide RNA or the second guide RNA, in some embodiments, binds to the recombinant nuclease and targets the recombinant nuclease to a specific location within the target gene such as at a location within the sense strand or the antisense strand of the target gene that is or includes the cleavage site. In some embodiments, the recombinant nuclease is a Cas protein from any bacterial species, or is a functional fragment thereof. In some embodiments, the Cas protein is Cas9 nuclease. Cas9 can, in some embodiments, be a Cas9 or functional fragment thereof from any bacterial species. See, e.g., Makarova et al. Nature Reviews, Microbiology, 9:467-477 (2011), including supplemental information, hereby incorporated by reference in its entirety. In some embodiments, the Cas9 is from Streptococcus pyogenes (SpCas9). In some embodiments, the Cas9 is from Staphylococcus aureus (SaCas9). In some embodiments, the Cas9 is from Neisseria meningitidis (NmeCas9). In some embodiments, the Cas9 is from Campylobacter jejuni (CjCas9). In some embodiments, the Cas9 is from Streptococcus thermophilis (StCas9).

In some embodiments, the Cas9 is from Streptococcus pyogenes (SpCas9) and comprises one or more mutations in the RuvC catalytic domain or the HNH catalytic domain. In some embodiments, the one or more mutations in the RuvC catalytic domain or the HNH catalytic domain inactivates the catalytic activity of the domain. In some embodiments, the recombinant nuclease has RuvC activity but does not have HNH activity. In some embodiments, the recombinant nuclease does not have RuvC activity but does have HNH activity. In some embodiments, the Cas9 is from Streptococcus pyogenes (SpCas9) and comprises one or more mutations selected from the group consisting of D10A, H840A, H854A, and H863A. In some embodiments, the Cas9 is from Streptococcus pyogenes (SpCas9) and comprises one or more mutations in the RuvC I, RuvC II, or RuvC III motifs. In some embodiments, the Cas9 is from Streptococcus pyogenes (SpCas9) and comprises a mutation in the RuvC I motif. In some embodiments, the Cas9 is from Streptococcus pyogenes (SpCas9) and comprises a D10A mutation in the RuvC I motif. In some embodiments, the Cas9 is from Streptococcus pyogenes (SpCas9) and comprises one or more mutations in the HNH catalytic domain. In some embodiments, the one or more mutations in the HNH catalytic domain is selected from the group consisting of H840A, H854A, and H863A. In some embodiments, the Cas9 is from Streptococcus pyogenes (SpCas9) and comprises a H840A mutation in the HNH catalytic domain. In some embodiments, the Cas9 is from Streptococcus pyogenes (SpCas9) and comprises a H840A mutation. In some embodiments, the Cas9 is from Streptococcus pyogenes (SpCas9) and comprises a D10A mutation. In some embodiments, the Cas9 is from Streptococcus pyogenes (SpCas9) and comprises one or more mutations selected from the group consisting of N497A, R661A, Q695A, and Q926A. In some embodiments, the Cas9 is from Streptococcus pyogenes (SpCas9) and comprises one or more mutations selected from the group consisting of R780A, K810A, K855A, H982A, K1003A, R1060A, and K848A. In some embodiments, the Cas9 is from Streptococcus pyogenes (SpCas9) and comprises one or more mutations selected from the group consisting of N692A, M694A, Q695A, and H698A. In some embodiments, the Cas9 is from Streptococcus pyogenes (SpCas9) and comprises one or more mutations selected from the group consisting of M495V, Y515N, K526E, and R661Q. In some embodiments, the Cas9 is from Streptococcus pyogenes (SpCas9) and comprises one or more mutations selected from the group consisting of F539S, M763I, and K890N. In some embodiments, the Cas9 is from Streptococcus pyogenes (SpCas9) and comprises one or more mutations selected from the group consisting of E480K, E543D, E1219V, A262T, S409I, M694I, E108G, S217A.

In some embodiments, the Cas9 is from Streptococcus pyogenes (SaCas9). In some embodiments, the SaCas9 is wild type SaCas9. In some embodiments, the SaCas9 comprises one or more mutations in REC3 domain. In some embodiments, the SaCas9 comprises one or more mutations in REC1 domain. In some embodiments, the SaCas9 comprises one or more mutations selected from the group consisting of N260D, N260Q, N260E, Q414A, Q414L. In some embodiments, the SaCas9 comprises one or more mutations in the recognition lobe. In some embodiments, the SaCas9 comprises one or more mutations selected from the group consisting of R245A, N413A, N419A. In some embodiments, the SaCas9 comprises one or more mutations in the RuvC-III domain. In some embodiments, the SaCas9 comprises a R654A mutation.

In some embodiments, the Cas protein is Cas12. In some embodiments, the Cas protein is Cas12a (i.e. cpf1). In some embodiments, the Cas12a is from the group consisting of Francisella novicida U112 (FnCas12a), Acidaminococcus sp. BV3L6 (AsCas12a), Moraxella bovoculi AAX11_00205 (Mb3Cas12a), Lachnospiraceae bacterium ND2006 (LbCas12a), Thiomicrospira sp. Xs5 (TsCas12a), Moraxella bovoculi AAX08_00205 (Mb2Cas12a), and Butyrivibrio sp. NC3005 (BsCas12a). In some embodiments, the Cas12a recognizes a T-rich 5′ protospacer adjacent motif (PAM). In some embodiments, the Cas12a processes its own crRNA without requiring a transactivating crRNA (tracrRNA). In some embodiments, the Cas12a processes both RNase and DNase activity. In some embodiments, the Cas12a is a split Cas12a platform, consisting of N-terminal and C-terminal fragments of Cas12a. In some embodiments, the split Cas12a platform is from Lachnospiraceae bacterium.

In some embodiments, the particles containing a Cas nuclease (e.g., Cas9) further comprise one or more CRISPR-Cas system guide RNA(s) for targeting a desired target gene. In some embodiments, the CRISPR guide RNAs are efficiently encapsulated in the CAS-containing particles. In some embodiments, the provided particles (e.g., lentiviral particles) further comprise a targeting nucleic acid.

In some embodiments, the lipid particle further comprises a polynucleotide per se, i.e. a polynucleotide that does not encode for a heterologous protein. In some embodiments, the polynucleotide per se is associated with a gene editing system. For example, a lipid particle may comprise a guide RNA (gRNA), such as a single guide RNA (sgRNA).

In some embodiments, the one or more agent(s) (e.g., one or more exogenous agent and/or heterologous protein) comprise, or are used in combination with, a guide RNA, e.g., single guide RNA (sgRNA), for inducing a DSB at the cleavage site. In some embodiments, the one or more agent(s) comprise, or are used in combination with, more than one guide RNA, e.g., a first sgRNA and a second sgRNA, for inducing a DSB at the cleavage site through a SSB on each strand. In some embodiments, the one or more agent(s) (e.g., the heterologous protein) can be used in combination with a donor template, e.g., a single-stranded DNA oligonucleotide (ssODN), for HDR-mediated integration of the donor template into the target gene, such as at the targeting sequence. In some embodiments, the one or more agent(s) (e.g., one or more exogenous agent and/or heterologous protein) can be used in combination with a donor template, e.g., an ssODN, and a guide RNA, e.g., a sgRNA, for HDR-mediated integration of the donor template into the target gene, such as at the targeting sequence. In some embodiments, the one or more agent(s) (e.g., one or more exogenous agent and/or heterologous protein) can be used in combination with a donor template, e.g., an ssODN, and a first guide RNA, e.g., a first sgRNA, and a second guide RNA, e.g., a second sgRNA, for HDR-mediated integration of the donor template into the target gene, such as at the targeting sequence.

In particular embodiments, the genome-modifying agent is a Cas protein, such as Cas9. In some embodiments, delivery of the CRISPR/Cas can be used to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. In some embodiments, a dCas9 version of the CRISPR/Cas9 system can be used to target protein domains for transcriptional regulation, epigenetic modification, and microscopic visualization of specific genome loci.

In some embodiments, the genome-modifying agent, e.g., Cas9, is targeted to the cleavage site by interacting with a guide RNA, e.g., sgRNA, that hybridizes to a DNA sequence that immediately precedes a Protospacer Adjacent Motif (PAM) sequence. In general, a guide RNA, e.g., sgRNA, is any nucleotide sequence comprising a sequence, e.g., a crRNA sequence, that has sufficient complementarity with a target gene sequence to hybridize with the target gene sequence at the cleavage site and direct sequence-specific binding of the recombinant nuclease to a portion of the target gene that includes the cleavage site. Full complementarity (100%) is not necessarily required, so long as there is sufficient complementarity to cause hybridization and promote formation of a complex, e.g., CRISPR complex, that includes the recombinant nuclease, e.g., Cas9, and the guide RNA, e.g., sgRNA. In some embodiments, the cleavage site is situated at a site within the target gene that is homologous to the sequence of the guide RNA, e.g., sgRNA. In some embodiments, the cleavage site is situated approximately 3 nucleotides upstream of the PAM sequence. In some embodiments, the cleavage site is situated approximately 3 nucleotides upstream of the juncture between the guide RNA and the PAM sequence. In some embodiments, the cleavage site is situated 3 nucleotides upstream of the PAM sequence. In some embodiments, the cleavage site is situated 4 nucleotides upstream of the PAM sequence.

In some embodiments, the one or more agent(s) (e.g., one or more exogenous agent and/or heterologous protein) capable of inducing a DSB comprise a fusion protein comprising a DNA binding domain and a DNA cleavage domain. In some embodiments, the DNA cleavage domain is or comprises a recombinant nuclease. In some embodiments, the fusion protein is a TALEN comprising a DNA binding domain and a DNA cleavage domain. In some embodiments, the DNA binding domain is a transcription activator-like (TAL) effector DNA binding domain. In some embodiments, the TAL effector DNA binding domain is from Xanthomonas bacteria. In some embodiments, the DNA cleavage domain is a Fokl nuclease domain. In some embodiments, the TAL effector DNA binding domain is engineered to target a specific target sequence, e.g., a portion of a target gene that includes a cleavage site.

In some embodiments, the fusion protein is a zinc finger nuclease (ZFN) comprising a zinc finger DNA binding domain and a DNA cleavage domain. In some embodiments, the DNA cleavage domain is a Fokl nuclease domain. In some embodiments, the zinc finger DNA binding domain is engineered to target a specific target sequence, e.g., a portion of a target gene, that includes a cleavage site, such as the targeting sequence.

In some embodiments, the provided lipid particles can be for use in a method to deliver an exogenous agent which involves introducing, into a cell, one or more agent(s) (e.g., one or more exogenous agent and/or heterologous protein) capable of inducing a SSB at a cleavage site within the sense strand and a SSB at a cleavage site within the antisense strand of an endogenous target gene in the cell.

In some embodiments, the cleavage site in the sense strand is less than 400, less than 350, less than 300, less than 250, less than 200, less than 175, less than 150, less than 125, less than 100, less than 90, less than 80, less than 75, less than 70, less than 65, less than 60, less than 55, less than 50, less than 45, less than 40, or less than 35 nucleotides from the nucleotide that is complementary to the cleavage site in the antisense strand. In some embodiments, the cleavage site in the antisense strand is less than 400, less than 350, less than 300, less than 250, less than 200, less than 175, less than 150, less than 125, less than 100, less than 90, less than 80, less than 75, less than 70, less than 65, less than 60, less than 55, less than 50, less than 45, less than 40, or less than 35 nucleotides from the nucleotide that is complementary to the cleavage site in the sense strand. In some embodiments, the cleavage site in the sense strand is between 20 and 400, 20 and 350, 20 and 300, 20 and 250, 20 and 200, 20 and 150, 20 and 125, 20 and 100, 20 and 90, 20 and 80, 20 and 70, 30 and 400, 30 and 350, 30 and 300, 30 and 250, 30 and 200, 30 and 150, 30 and 125, 30 and 100, 30 and 90, 30 and 80, 30 and 70, 40 and 400, 40 and 350, 40 and 300, 40 and 250, 40 and 200, 40 and 150, 40 and 125, 40 and 100, 40 and 90, 40 and 80, or 40 and 70 nucleotides from the nucleotide that is complementary to the cleavage site in the antisense strand. In some embodiments, the cleavage site in the antisense strand is between 20 and 400, 20 and 350, 20 and 300, 20 and 250, 20 and 200, 20 and 150, 20 and 125, 20 and 100, 20 and 90, 20 and 80, 20 and 70, 30 and 400, 30 and 350, 30 and 300, 30 and 250, 30 and 200, 30 and 150, 30 and 125, 30 and 100, 30 and 90, 30 and 80, 30 and 70, 40 and 400, 40 and 350, 40 and 300, 40 and 250, 40 and 200, 40 and 150, 40 and 125, 40 and 100, 40 and 90, 40 and 80, or 40 and 70 nucleotides from the nucleotide that is complementary to the cleavage site in the sense strand.

In some embodiments, the one or more agent(s) (e.g., one or more exogenous agent and/or heterologous protein) capable of inducing a SSB at a cleavage site within the sense strand and a SSB at a cleavage site within the antisense strand comprise a recombinant nuclease. In some embodiments, the recombinant nuclease includes a recombinant nuclease that induces the SSB in the sense strand, and a recombinant nuclease that induced the SSB in the antisense strand, and both of which recombinant nucleases are referred to as the recombinant nuclease. Accordingly, in some embodiments, the method involves introducing, into a cell, one or more agent(s) (e.g., the one or more exogenous agent and/or heterologous protein) comprising a recombinant nuclease for inducing a SSB at a cleavage site in the sense strand and a SSB at a cleavage site in the antisense strand within an endogenous target gene in the cell. Although, in some embodiments, it is described that “a” “the” recombinant nuclease induces a SSB in the antisense strand a SSB in the sense strand, it is to be understood that this includes situations where two of the same recombinant nuclease is used, such that one of the recombinant nuclease induces the SSB in the sense strand and the other recombinant nuclease induces the SSB in the antisense strand. In some embodiments, the recombinant nuclease that induces the SSB lacks the ability to induce a DSB by cleaving both strands of double stranded DNA.

In some embodiments, the one or more agent(s) capable of inducing a SSB comprise a recombinant nuclease and a first guide RNA, e.g., a first sgRNA, and a second guide RNA, e.g., a second sgRNA.

In some embodiments, the genome-modifying agent is a Cas protein, a transcription activator-like effector nuclease (TALEN), or a zinc finger nuclease (ZFN). In some embodiments, the recombinant nuclease is a Cas nuclease. In some embodiments, the recombinant nuclease is a TALEN. In some embodiments, the recombinant nuclease is a ZFN.

In some embodiments, the one or more agent(s) capable of inducing a SSB at a cleavage site within the sense strand and a SSB at a cleavage site within the antisense strand comprise a fusion protein comprising a DNA binding domain and a DNA cleavage domain. In some embodiments, the DNA cleavage domain is or comprises a recombinant nuclease. In some embodiments, the fusion protein is a TALEN comprising a DNA binding domain and a DNA cleavage domain. In some embodiments, the DNA binding domain is a transcription activator-like (TAL) effector DNA binding domain. In some embodiments, the TAL effector DNA binding domain is from Xanthomonas bacteria. In some embodiments, the DNA cleavage domain is a Fokl nuclease domain. In some embodiments, the TAL effector DNA binding domain is engineered to target a specific target sequence, e.g., a portion of a target gene that includes a cleavage site. In some embodiments, the fusion protein is a zinc finger nuclease (ZFN) comprising a zinc finger DNA binding domain and a DNA cleavage domain. In some embodiments, the DNA cleavage domain is a Fokl nuclease domain. In some embodiments, the zinc finger DNA binding domain is engineered to target a specific target sequence, e.g., a portion of a target gene that includes a cleavage site, such as the targeting sequence.

In some embodiments, the one or more agent(s) capable of inducing a SSB at a cleavage site within the sense strand and a SSB at a cleavage site within the antisense strand involve use of the CRISPR/Cas gene editing system. In some embodiments, the one or more agent(s) comprise a recombinant nuclease.

In some embodiments, the genome-modifying agent is a Cas protein. In some embodiments, the Cas protein comprises one or more mutations such that the Cas protein is converted into a nickase that lacks the ability to cleave both strands of a double stranded DNA molecule. In some embodiments, the Cas protein comprises one or more mutations such that the Cas protein is converted into a nickase that is able to cleave only one strand of a double stranded DNA molecule. For example, Cas9, which is normally capable of inducing a double strand break, can be converted into a Cas9 nickase, which is capable of inducing a single strand break, by mutating one of two Cas9 catalytic domains: the RuvC domain, which comprises the RuvC I, RuvC II, and RuvC III motifs, or the NHN domain. In some embodiments, the Cas protein comprises one or more mutations in the RuvC catalytic domain or the HNH catalytic domain. In some embodiments, the genome-modifying protein is a recombinant nuclease that has been modified to have nickase activity. In some embodiments, the recombinant nuclease cleaves the strand to which the guide RNA, e.g., sgRNA, hybridizes, but does not cleave the strand that is complementary to the strand to which the guide RNA, e.g., sgRNA, hybridizes. In some embodiments, the recombinant nuclease does not cleave the strand to which the guide RNA, e.g., sgRNA, hybridizes, but does cleave the strand that is complementary to the strand to which the guide RNA, e.g., sgRNA, hybridizes.

In some embodiments, the lipid particle further comprises a guide RNA (gRNA), such as a single guide RNA (sgRNA). Thus, in some embodiments, the heterologous agent comprises a guide RNA (gRNA). In some embodiments, the gRNA is a single guide RNA (sgRNA).

In some embodiments, the genome-modifying protein, e.g., Cas9, is targeted to the cleavage site by interacting with a guide RNA, e.g., a first guide RNA, such as a first sgRNA, or a second guide RNA, such as a second sgRNA, that hybridizes to a DNA sequence on the sense strand or the antisense strand that immediately precedes a Protospacer Adjacent Motif (PAM) sequence.

In some embodiments, the genome-modifying agent, e.g., Cas9, is targeted to the cleavage site on the sense strand by interacting with a first guide RNA, e.g., first sgRNA, that hybridizes to a sequence on the sense strand that immediately precedes a PAM sequence. In some embodiments, the genome-modifying agent, e.g., Cas9, is targeted to the cleavage site on the antisense strand by interacting with a second guide RNA, e.g., second sgRNA, that hybridizes to a sequence on the antisense strand that immediately precedes a PAM sequence.

In some embodiments, the first guide RNA, e.g., first sgNA, that is specific to the sense strand of a target gene of interest is used to target the recombinant nuclease, e.g., Cas9, to induce a SSB at a cleavage site within the sense strand of the target gene. In some embodiments, the first guide RNA, e.g., first sgNA, that is specific to the antisense strand of a target gene of interest is used to target the recombinant nuclease, e.g., Cas9, to induce a SSB at a cleavage site within the antisense strand of the target gene.

In some embodiments, the second guide RNA, e.g., second sgNA, that is specific to the sense strand of a target gene of interest used to target the recombinant nuclease, e.g., Cas9, to induce a SSB at a cleavage site within the sense strand of the target gene. In some embodiments, the second guide RNA, e.g., second sgNA, that is specific to the antisense strand of a target gene of interest is used to target the recombinant nuclease, e.g., Cas9, to induce a SSB at a cleavage site within the antisense strand of the target gene.

In some embodiments, the first guide RNA, e.g., first sgNA, that is specific to the sense strand of a target gene of interest is used to target the recombinant nuclease, e.g., Cas9, to induce a SSB at a cleavage site within the sense strand of the target gene; and the second guide RNA, e.g., second sgNA, that is specific to the antisense strand of a target gene of interest is used to target the recombinant nuclease, e.g., Cas9, to induce a SSB at a cleavage site within the antisense strand of the target gene.

In some embodiments, the first guide RNA, e.g., first sgNA, that is specific to the antisense strand of a target gene of interest is used to target the recombinant nuclease, e.g., Cas9, to induce a SSB at a cleavage site within the antisense strand of the target gene; and the second guide RNA, e.g., second sgNA, that is specific to the sense strand of a target gene of interest is used to target the recombinant nuclease, e.g., Cas9, to induce a SSB at a cleavage site within the sense strand of the target gene. In general, a guide RNA, e.g., a first guide RNA, such as a first sgRNA, or a second guide RNA, such as a second sgRNA, is any nucleotide sequence comprising a sequence, e.g., a crRNA sequence, that has sufficient complementarity with a target gene sequence to hybridize with the target gene sequence at the cleavage site and direct sequence-specific binding of the recombinant nuclease to a portion of the target gene that includes the cleavage site. Full complementarity (100%) is not necessarily required, so long as there is sufficient complementarity to cause hybridization and promote formation of a complex, e.g., CRISPR complex, that includes the recombinant nuclease, e.g., Cas9, and the guide RNA, e.g., the first guide RNA, such as the first sgRNA, or the second guide RNA, such as the second sgRNA.

In some embodiments, the cleavage site is situated at a site within the target gene that is homologous to a sequence comprised within the guide RNA, e.g., sgRNA. In some embodiments, the cleavage site of the sense strand is situated at a site within the sense strand of the target gene that is homologous to a sequence comprised within the first guide RNA, e.g., the first sgRNA. In some embodiments, the cleavage site of the antisense strand is situated at a site within the antisense strand of the target gene that is homologous to a sequence comprised within the first guide RNA, e.g., the first sgRNA. In some embodiments, the cleavage site of the sense strand is situated at a site within the sense strand of the target gene that is homologous to a sequence comprised within the second guide RNA, e.g., the second sgRNA. In some embodiments, the cleavage site of the antisense strand is situated at a site within the antisense strand of the target gene that is homologous to a sequence comprised within the second guide RNA, e.g., the second sgRNA. In some embodiments, the cleavage site of the sense strand is situated at a site within the sense strand of the target gene that is homologous to a sequence comprised within the first guide RNA, e.g., the first sgRNA; and the cleavage site of the antisense strand is situated at a site within the antisense strand of the target gene that is homologous to a sequence comprised within the second guide RNA, e.g., the second sgRNA. In some embodiments, the cleavage site of the antisense strand is situated at a site within the antisense strand of the target gene that is homologous to a sequence comprised within the first guide RNA, e.g., the first sgRNA; and the cleavage site of the sense strand is situated at a site within the sense strand of the target gene that is homologous to a sequence comprised within the second guide RNA, e.g., the second sgRNA. In some embodiments, the cleavage site of the antisense strand is situated at a site within the antisense strand of the target gene that is homologous to a sequence comprised within the second guide RNA, e.g., the second sgRNA; and the cleavage site of the sense strand is situated at a site within the sense strand of the target gene that is homologous to a sequence comprised within the first guide RNA, e.g., the first sgRNA.

In some embodiments, the sense strand comprises the targeting sequence, and the targeting sequence includes the SNP and a protospacer adjacent motif (PAM) sequence. In some embodiments, the sense strand comprises the targeting sequence, and the targeting sequence includes the SNP and a protospacer adjacent motif (PAM) sequence; and the antisense strand comprises a sequence that is complementary to the targeting sequence and includes a PAM sequence. In some embodiments, the antisense strand comprises the targeting sequence, and the targeting sequence includes the SNP and a protospacer adjacent motif (PAM) sequence. In some embodiments, the antisense strand comprises the targeting sequence, and the targeting sequence includes the SNP and a protospacer adjacent motif (PAM) sequence; and the sense strand comprises a sequence that is complementary to the targeting sequence and includes a PAM sequence.

In some embodiments, the cleavage site on the sense strand and/or the antisense strand is situated approximately 3 nucleotides upstream of the PAM sequence. In some embodiments, the cleavage site on the sense strand and/or the antisense strand is situated approximately 3 nucleotides upstream of the juncture between the guide RNA and the PAM sequence. In some embodiments, the cleavage site on the sense strand and/or the antisense strand is situated 3 nucleotides upstream of the PAM sequence. In some embodiments, the cleavage site on the sense strand and/or the antisense strand is situated 4 nucleotides upstream of the PAM sequence.

In some embodiments, the PAM sequence that is recognized by a recombinant nuclease is in the sense strand. In some embodiments, the PAM sequence that is recognized by a recombinant nuclease is in the antisense strand. In some embodiments, the PAM sequence that is recognized by a recombinant nuclease is in the sense strand and is in the antisense strand. In some embodiments, the PAM sequence on the sense strand and the PAM sequence on the antisense strand are outwardly facing. In some embodiments, the PAM sequence on the sense strand and the PAM sequence on the antisense strand comprise the same nucleic acid sequence, which can be any PAM sequence disclosed herein. In some embodiments, the PAM sequence on the sense strand and the PAM sequence on the antisense strand each comprise a different nucleic acid sequence, each of which can be any of the PAM sequences disclosed herein.

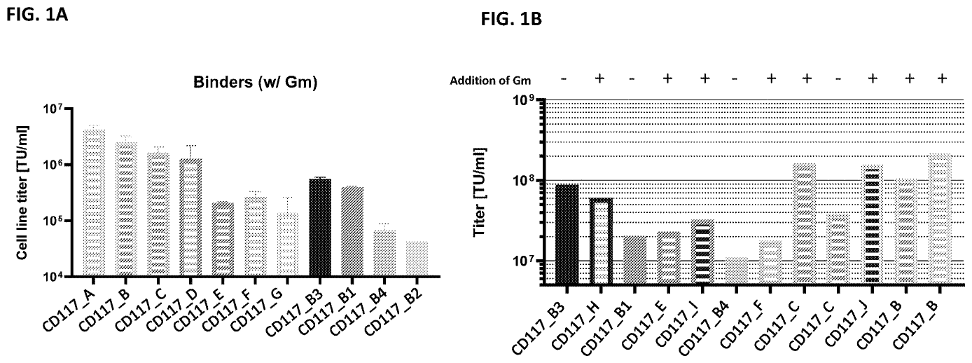

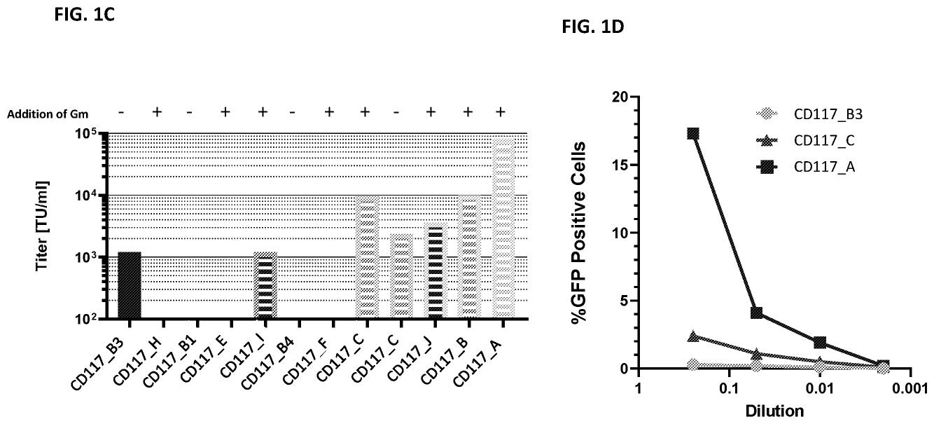

In some embodiments, the PAM sequence that is recognized by a recombinant nuclease, e.g., Cas9, differs depending on the particular recombinant nuclease and the bacterial species it is from Methods for designing guide RNAs, e.g., sgRNAs, and their exemplary targeting sequences, e.g., crRNA sequences, can include those described in, e.g., International PCT Pub. Nos. WO2015/161276, WO2017/193107, and WO2017/093969. Exemplary guide RNA structures, including particular domains, are described in WO2015/161276, e.g., in FIGS. 1A-1G therein. Since guide RNA is an RNA molecule, it will comprise the base uracil (U), while any DNA encoding the guide RNA molecule will comprise the base thymine (T). In some embodiments, the guide RNA, e.g., sgRNA, comprises a CRISPR targeting RNA sequence (crRNA) and a trans-activating crRNA sequence (tracrRNA). In some embodiments, the first guide RNA, e.g., the first sgRNA, and the second guide RNA, e.g., the second sgRNA, each comprise a crRNA and a tracrRNA. In some embodiments, the guide RNA, e.g., sgRNA, is an RNA comprising, from 5′ to 3′: a crRNA sequence and a tracrRNA sequence. In some embodiments, each of the first guide RNA, e.g., first sgRNA, and the second guide RNA, e.g., second sgRNA, is an RNA comprising, from 5′ to 3′: a crRNA sequence and a tracrRNA sequence. In some embodiments, the crRNA and tracrRNA do not naturally occur together in the same sequence.

In some embodiments, the crRNA comprises a nucleotide sequence that is homologous, e.g., is at least 80%, 85%, 90%, 95%, 96%, 97%, 98%, or 99% homologous, or is 100% homologous, to a portion of the target gene that includes the cleavage site. In some embodiments, the crRNA comprises a nucleotide sequence that is 100% homologous to a portion of the target gene that includes the cleavage site. In some embodiments, the portion of the target gene that includes the cleavage site is a portion of the sense strand of the target gene that includes the cleavage site. In some embodiments, the portion of the target gene that includes the cleavage site is a portion of the antisense strand of the target gene that includes the cleavage site.

In some embodiments, the sgRNA comprises a crRNA sequence that is homologous to a sequence in the target gene that includes the cleavage site. In some embodiments, the first sgRNA comprises a crRNA sequence that is homologous to a sequence in the sense strand of the target gene that includes the cleavage site; and/or the second sgRNA comprises a crRNA sequence that is homologous to a sequence in the antisense strand of the target gene that includes the cleavage site. In some embodiments, the first sgRNA comprises a crRNA sequence that is homologous to a sequence in the antisense strand of the target gene that includes the cleavage site; and/or the second sgRNA comprises a crRNA sequence that is homologous to a sequence in the sense strand of the target gene that includes the cleavage site.

In some embodiments, the crRNA sequence has 100% sequence identity to a sequence in the target gene that includes the cleavage site. In some embodiments, the crRNA sequence of the first sgRNA has 100% sequence identity to a sequence in the sense strand of the target gene that includes the cleavage site; and/or the crRNA sequence of the second sgRNA has 100% sequence identity to a sequence in the antisense strand of the target gene that includes the cleavage site. In some embodiments, the crRNA sequence of the first sgRNA has 100% sequence identity to a sequence in the antisense strand of the target gene that includes the cleavage site; and/or the crRNA sequence of the second sgRNA has 100% sequence identity to a sequence in the sense strand of the target gene that includes the cleavage site.

Guidance on the selection of crRNA sequences can be found, e.g., in Fu Y et al., Nat Biotechnol 2014 (doi: 10.1038/nbt.2808) and Sternberg S H et al., Nature 2014 (doi: 10.1038/nature13011). Examples of the placement of crRNA sequences within the guide RNA, e.g., sgRNA, structure include those described in WO2015/161276, e.g., in FIGS. 1A-1G therein.

Reference to “the crRNA” is to be understood as also including reference to the crRNA of the first sgRNA and the crRNA of the second sgRNA, each independently. Thus, embodiments referring to “the crRNA” is to be understood as independently referring to embodiments of (i) the crRNA, (ii) the crRNA of the first sgRNA, and (iii) the crRNA of the second sgRNA. In some embodiments, the crRNA is 15-27 nucleotides in length, i.e., the crRNA is 15, 16, 17, 18, 19, 20, 21, 22, 23, 24, 25, 26, or 27 nucleotides in length. In some embodiments, the crRNA is 18-22 nucleotides in length. In some embodiments, the crRNA is 19-21 nucleotides in length. In some embodiments, the crRNA is 20 nucleotides in length.

In some embodiments, the crRNA is homologous to a portion of a target gene that includes the cleavage site. In some embodiments, the crRNA is homologous to a portion of the sense strand of the target gene that includes the cleavage site. In some embodiments, the crRNA is homologous to a portion of the antisense strand of the target gene that includes the cleavage site. In some embodiments, the crRNA of the first sgRNA is homologous to a portion of the sense strand of the target gene that includes the cleavage site; and the crRNA of the second sgRNA is homologous to a portion of the antisense strand of the target gene that includes the cleavage site.

In some embodiments, the crRNA is homologous to a portion of the antisense strand of a target gene that includes the cleavage site. In some embodiments, the crRNA is homologous to a portion of the sense strand of the target gene that includes the cleavage site. In some embodiments, the crRNA of the first sgRNA is homologous to a portion of the antisense strand of the target gene that includes the cleavage site; and the crRNA of the second sgRNA is homologous to a portion of the sense strand of the target gene that includes the cleavage site.

In some embodiments, the crRNA is homologous to a portion of a target gene that includes the cleavage site, and is 15-27 nucleotides in length, i.e., the crRNA is 15, 16, 17, 18, 19, 20, 21, 22, 23, 24, 25, 26, or 27 nucleotides in length. In some embodiments, the portion of the target gene that includes the cleavage site is on the sense strand. In some embodiments, the portion of the target gene that includes the cleavage site is on the antisense strand.

In some embodiments, the crRNA is homologous to a portion, i.e., sequence, in the sense strand or the antisense strand of the target gene that includes the cleavage site and is immediately upstream of the PAM sequence.

In some embodiments, the tracrRNA sequence may be or comprise any sequence for tracrRNA that is used in any CRISPR/Cas9 system known in the art. Reference to “the tracrRNA” is to be understood as also including reference to the tracrRNA of the first sgRNA and the tracrRNA of the second sgRNA, each independently. Thus, embodiments referring to “the tracrRNA” is to be understood as independently referring to embodiments of (i) the tracrRNA, (ii) the tracrRNA of the first sgRNA, and (iii) the tracrRNA of the second sgRNA. Exemplary CRISPR/Cas9 systems, sgRNA, crRNA, and tracrRNA, and their manufacturing process and use include those described in, e.g., International PCT Pub. Nos. WO2015/161276, WO2017/193107 and WO2017/093969, and those described in, e.g., U.S. Patent Application Publication Nos. 20150232882, 20150203872, 20150184139, 20150079681, 20150073041, 20150056705, 20150031134, 20150020223, 20140357530, 20140335620, 20140310830, 20140273234, 20140273232, 20140273231, 20140256046, 20140248702, 20140242700, 20140242699, 20140242664, 20140234972, 20140227787, 20140189896, 20140186958, 20140186919, 20140186843, 20140179770, 20140179006, 20140170753, 20140093913, and 20140080216.

In some embodiments, the heterologous protein is associated with base editing. Base editors (BEs) are typically fusions of a Cas (“CRISPR-associated”) domain and a nucleobase modification domain (e.g., a natural or evolved deaminase, such as a cytidine deaminase that include APOBEC1 (“apolipoprotein B mRNA editing enzyme, catalytic polypeptide 1”), CDA (“cytidine deaminase”), and AID (“activation-induced cytidine deaminase”)) domains. In some cases, base editors may also include proteins or domains that alter cellular DNA repair processes to increase the efficiency and/or stability of the resulting single-nucleotide change.

In some aspects, currently available base editors include cytidine base editors (e.g., BE4) that convert target C•G to T•A and adenine base editors (e.g., ABE7.10) that convert target A•T to G•C. In some aspects, Cas9-targeted deamination was first demonstrated in connection with a Base Editor (BE) system designed to induce base changes without introducing double-strand DNA breaks. Further Rat deaminase APOBEC1 (rAPOBEC1) fused to deactivated Cas9 (dCas9) was used to successfully convert cytidines to thymidines upstream of the PAM of the sgRNA. In some aspects, this first BE system was optimized by changing the dCas9 to a “nickase” Cas9 D10A, which nicks the strand opposite the deaminated cytidine. Without being bound by theory, this is expected to initiate long-patch base excision repair (BER), where the deaminated strand is preferentially used to template the repair to produce a U: A base pair, which is then converted to T: A during DNA replication.

In some embodiments, the exogenous agent and/or heterologous protein is or encodes a base editor (e.g., a nucleobase editor). In some embodiments, the exogenous agent and/or heterologous protein is a nucleobase editor containing a first DNA binding protein domain that is catalytically inactive, a domain having base editing activity, and a second DNA binding protein domain having nickase activity, where the DNA binding protein domains are expressed on a single fusion protein or are expressed separately (e.g., on separate expression vectors). In some embodiments, the base editor is a fusion protein comprising a domain having base editing activity (e.g., cytidine deaminase or adenosine deaminase), and two nucleic acid programmable DNA binding protein domains (napDNAbp), a first comprising nickase activity and a second napDNAbp that is catalytically inactive, wherein at least the two napDNAbp are joined by a linker. In some embodiments, the base editor is a fusion protein that comprises a DNA domain of a CRISPR-Cas (e.g., Cas9) having nickase activity (nCas; nCas9), a catalytically inactive domain of a CRISPR-Cas protein (e.g., Cas9) having nucleic acid programmable DNA binding activity (dCas; e.g., dCas9), and a deaminase domain, wherein the dCas is joined to the nCas by a linker, and the dCas is immediately adjacent to the deaminase domain. In some embodiments, the base editor is a adenine-to-thymine or “ATBE” (or thymine-to-adenine or “TABE”) transversion base editors. Exemplary base editor and base editor systems include any as described in patent publication Nos. US20220127622, US20210079366, US20200248169, US20210093667, US20210071163, WO2020181202, WO2021158921, WO2019126709, WO2020181178, WO2020181195, WO2020214842, WO2020181193, which are hereby incorporated in their entirety.

In some embodiments, the exogenous agent and/or heterologous protein is one for use in target-primed reverse transcription (TPRT) or “prime editing”. In some embodiments, prime editing mediates targeted insertions, deletions, all 12 possible base-to-base conversions, and combinations thereof in human cells without requiring DSBs or donor DNA templates.

Prime editing is a genome editing method that directly writes new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PERNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same sequence as the endogenous strand of the target site to be edited (with the exception that it includes the desired edit). Through DNA repair and/or replication machinery, the endogenous strand of the target site is replaced by the newly synthesized replacement strand containing the desired edit. In some cases, prime editing may be thought of as a “search-and-replace” genome editing technology since the prime editors search and locate the desired target site to be edited, and encode a replacement strand containing a desired edit which is installed in place of the corresponding target site endogenous DNA strand at the same time. For example, prime editing can be adapted for conducting precision CRISPR/Cas-based genome editing in order to bypass double stranded breaks. In some embodiments, the heterologous protein is or encodes for a Cas protein-reverse transcriptase fusions or related systems to target a specific DNA sequence with a guide RNA, generate a single strand nick at the target site, and use the nicked DNA as a primer for reverse transcription of an engineered reverse transcriptase template that is integrated with the guide RNA. In some embodiments, the prime editor protein is paired with two prime editing guide RNAs (pegRNAs) that template the synthesis of complementary DNA flaps on opposing strands of genomic DNA, resulting in the replacement of endogenous DNA sequence between the PE-induced nick sites with pegRNA-encoded sequences.

In some embodiments, the exogenous agent and/or heterologous protein is or encodes for a primer editor that is a reverse transcriptase, or any DNA polymerase known in the art. Thus, in one aspect, the prime editor may comprise Cas9 (or an equivalent napDNAbp) which is programmed to target a DNA sequence by associating it with a specialized guide RNA (i.e., PEgRNA) containing a spacer sequence that anneals to a complementary protospacer in the target DNA. Such methods include any disclosed in Anzalone et al., (doi.org/10.1038/s41586-019-1711-4), or in PCT publication Nos. WO2020191248, WO2021226558, or WO2022067130, which are hereby incorporated in their entirety.

In some embodiments, the exogenous agent and/or heterologous protein is for use in Programmable Addition via Site-specific Targeting Elements (PASTE). In some aspects, PASTE is platform in which genomic insertion is directed via a CRISPR-Cas9 nickase fused to both a reverse transcriptase and serine integrase. As described in Ioannidi et al. (doi.org/10.1101/2021.11.01.466786), PASTE does not generate double stranded breaks, but allowed for integration of sequences as large as ~36 kb. In some embodiments, the serine integrase can be any known in the art. In some embodiments, the serine integrase has sufficient orthogonality such that PASTE can be used for multiplexed gene integration, simultaneously integrating at least two different genes at least two genomic loci. In some embodiments, PASTE has editing efficiencies comparable to or better than those of homology directed repair or non-homologous end joining based integration, with activity in nondividing cells and fewer detectable off-target events.

In some embodiments, the exogenous agent and/or heterologous protein is or encodes one or more polypeptides having an activity selected from the group consisting of: nuclease activity (e.g., programmable nuclease activity); nickase activity (e.g., programmable nickase activity); homing activity (e.g., programmable DNA binding activity); nucleic acid polymerase activity (e.g., DNA polymerase or RNA polymerase activity); integrase activity; recombinase activity; or base editing activity (e.g., cytidine deaminase or adenosine deaminase activity).

In some embodiments, delivery of the nuclease is by a provided vector encoding the nuclease (e.g. Cas).

In some embodiments, the provided lipid particles contain a nuclease protein and the nuclease protein is directly delivered to a target cell. Methods of delivering a nuclease protein include those as described, for example, in Cai et al. Elife, 2014, 3: e01911 and International patent publication No. WO2017068077. For instance, provided lipid particles comprise one or more Cas protein(s), such as Cas9. In some embodiments, the nuclease protein (e.g. Cas, such as Cas 9) is engineered as a chimeric nuclease protein with a viral structural protein (e.g. GAG) for packaging into the lipid particle (e.g. lentiviral vector particle, VLP, or gesicle). For instance, a chimeric Cas9-protein fusion with the structural GAG protein can be packaged inside a lipid particle. In some embodiments, the fusion protein is a cleavable fusion protein between (i) a viral structural protein (e.g. GAG) and (ii) a nuclease protein (e.g. Cas protein, such as Cas9). In some embodiments, the fusion protein is a cleavable fusion protein between (i) a viral matrix (MA) protein and (ii) a nuclease protein (e.g. Cas protein, such as Cas9). In some embodiments, the particle contains a nuclease protein (e.g., Cas protein, such as Cas 9) immediately downstream of the gag start codon.

In some embodiments, the provided lipid particles contain mRNA encoding a Cas nuclease (e.g., Cas9). In some embodiments, the provided lipid particles contain guide RNA (gRNA), such as a single guide RNA (sgRNA).

In some embodiments, a dCas9 version of the CRISPR/Cas9 system can be used to target protein domains for transcriptional regulation, epigenetic modification, and microscopic visualization of specific genome loci.

In some embodiments, the provided virus particles (e.g. lentiviral particles) containing a Cas nuclease (e.g. Cas9) further comprise, or is further complexed with, one or more CRISPR-Cas system guide RNA(s) for targeting a desired target gene. In some embodiments, the CRISPR guide RNAs are efficiently encapsulated in the CAS-containing viral particles. In some embodiments, the provided virus particles (e.g. lentiviral particles) further comprises, or is further complexed with a targeting nucleic acid.

### 4. Small Molecules

In some embodiments, the exogenous agent includes a small molecule, e.g., ions (e.g. Ca²⁺, Cl—, Fe²⁺), carbohydrates, lipids, reactive oxygen species, reactive nitrogen species, isoprenoids, signaling molecules, heme, polypeptide cofactors, electron accepting compounds, electron donating compounds, metabolites, ligands, and any combination thereof. In some embodiments the small molecule is a pharmaceutical that interacts with a target in the cell. In some embodiments the small molecule targets a protein in the cell for degradation. In some embodiments the small molecule targets a protein in the cell for degradation by localizing the protein to the proteasome. In some embodiments that small molecule is a proteolysis targeting chimera molecule (PROTAC).

In some embodiments, the exogenous agent includes a mixture of proteins, nucleic acids, or metabolites, e.g., multiple polypeptides, multiple nucleic acids, multiple small molecules; combinations of nucleic acids, polypeptides, and small molecules; ribonucleoprotein complexes (e.g. Cas9-gRNA complex); multiple transcription factors, multiple epigenetic factors, reprogramming factors (e.g. Oct4, Sox2, cMyc, and Klf4); multiple regulatory RNAs; and any combination thereof.

### III. Pharmaceutical Compositions

Provided herein are compositions containing the lipid particles herein, including lipid particles containing a retargeted attachment protein comprising (i) a paramyxovirus envelope attachment protein; and (ii) a targeting moiety directed to a first target molecule expressed on the surface of a target cell, and at least one paramyxovirus fusion protein. The pharmaceutical compositions can include any of the described lipid particles.

Also provided herein are compositions comprising any of the lipid particles described herein.

Also provided herein are compositions containing the lipid particles herein, including lipid particles containing a retargeted attachment protein, comprising: (a) a first paramyxovirus envelope attachment protein; and a first targeting moiety directed to a target molecule expressed on the surface of a target cell; (b) a second paramyxovirus envelope attachment protein; and a second targeting moiety directed to a target molecule expressed on the surface of a target cell; and (c) at least one paramyxovirus fusion protein.

Also provided herein are compositions containing the lipid particles herein, including lipid particles containing a retargeted attachment protein, comprising: (a) a first paramyxovirus envelope attachment protein; and a first targeting moiety directed to a target molecule expressed on the surface of a target cell; (b) a second paramyxovirus envelope attachment protein; and a second targeting moiety directed to a target molecule expressed on the surface of a target cell; (c) a third paramyxovirus envelope attachment protein, wherein the third paramyxovirus envelope attachment protein is a variant paramyxovirus envelope attachment protein comprising one or more mutations to reduce native tropism relative to the wild-type paramyxovirus envelope attachment protein not comprising the one or more mutations; and (d) at least one paramyxovirus fusion protein.

Also provided herein are compositions containing the lipid particles herein, including lipid particles containing a retargeted attachment protein comprising: (a) a first paramyxovirus envelope attachment protein; and a first targeting moiety directed to a target molecule expressed on the surface of a target cell; (b) a second paramyxovirus envelope attachment protein; and a second targeting moiety directed to a target molecule expressed on the surface of a target cell; (c) a third paramyxovirus envelope attachment protein, wherein the third paramyxovirus envelope attachment protein is a variant paramyxovirus envelope attachment protein comprising one or more mutations to reduce native tropism relative to the wild-type paramyxovirus envelope attachment protein not comprising the one or more mutations; and (d) at least one paramyxovirus fusion protein; and optionally one or more additional paramyxovirus envelope attachment proteins and one or more additional targeting moieties directed to a target molecule expressed on the surface of a target cell.

The pharmaceutical compositions provided herein can include any of the described lipid particles.

The present disclosure also provides, in some aspects, a pharmaceutical composition comprising the composition described herein and pharmaceutically acceptable carrier.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular lipid particle and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

In some embodiments, the lipid particle meets a pharmaceutical or good manufacturing practices (GMP) standard. In some embodiments, the lipid particle is made according to good manufacturing practices (GMP). In some embodiments, the lipid particle has a pathogen level below a predetermined reference value, e.g., is substantially free of pathogens. In some embodiments, the lipid particle has a contaminant level below a predetermined reference value, e.g., is substantially free of contaminants. In some embodiments, the lipid particle has low immunogenicity.

In some embodiments, formulations of the pharmaceutical compositions described herein may be prepared by any method known or hereafter developed in the art of pharmacology. In some embodiments, preparatory methods include the step of bringing the active ingredient into association with a carrier or one or more other accessory ingredients, and then, if necessary or desirable, shaping or packaging the product into a desired single- or multi-dose unit.

In some embodiments, a “unit dose” is a discrete amount of the pharmaceutical composition comprising a predetermined amount of the active ingredient. In some embodiments, the amount of the active ingredient is generally equal to the dosage of the active ingredient that would be administered to a subject or a convenient fraction of such a dosage such as, for example, one-half or one-third of such a dosage. In some embodiments, the unit dosage form may be for a single daily dose or one of multiple daily doses (e.g., about 1 to 4 or more times per day). In some embodiments, when multiple daily doses are used, the unit dosage form may be the same or different for each dose.

In some embodiments, the lipid particle containing the variant NiV-G is a viral vector or virus-like particle (e.g., Section III). In some embodiments, the compositions provided herein can be formulated in dosage units of genome copies (GC). Suitable method for determining GC have been described and include, e.g., qPCR or digital droplet PCR (ddPCR) as described in, e.g., M. Lock et al, Hu Gene Therapy Methods, Hum Gene Ther Methods 25 (2): 115-25. 2014, which is incorporated herein by reference. In some embodiments, the dosage of administration of a viral vector or virus-like particle is from about 10⁴ to about 10¹⁰ GC units, inclusive. In some embodiments, the dosage of administration of a viral vector or virus-like particle is from about 10⁹ to about 10¹⁵ GC units, inclusive. In some embodiments, the dosage of administration of a viral vector or virus-like particle is from about 10⁵ to about 10⁹ GC units, inclusive. In some embodiments, the dosage of administration of a viral vector or virus-like particle is from about 10⁶ to about 10⁹ GC units, inclusive. In some embodiments, the dosage of administration of a viral vector or virus-like particle is from about 10¹² to about 10¹⁴ GC units, inclusive. In some embodiments, the dosage of administration is 1.0×10⁹ GC units, 5.0×10⁹ GC units, 1.0×10¹⁰ GC units, 5.0×10¹⁰ GC units, 1.0×10¹¹ GC units, 5.0×10¹¹ GC units, 1.0×10¹² GC units, 5.0×10¹² GC units, or 1.0×10¹³ GC units, 5.0×10¹³ GC units, 1.0×10¹⁴ GC units, 5.0×10¹⁴ GC units, or 1.0×10¹⁵ GC units.

In some embodiments, the dosage of administration of a viral vector or virus-like particle is from about 10⁴ to about 10¹⁰ infectious units, inclusive. In some embodiments, the dosage of administration of a viral vector or virus-like particle is from about 10⁹ to about 10¹⁵ infectious units, inclusive In some embodiments, the dosage of administration of a viral vector or virus-like particle is from about 10⁵ to about 10⁹ infectious units. In some embodiments, the dosage of administration of a viral vector or virus-like particle is from about 10⁶ to about 10⁹ infectious units. In some embodiments, the dosage of administration of a viral vector or virus-like particle is from about 10¹² to about 10¹⁴ infectious units, inclusive. In some embodiments, the dosage of administration is 1.0×10⁹ infectious units, 5.0×10⁹ infectious units, 1.0×10¹⁰ infectious units, 5.0×10¹⁰ infectious units, 1.0×10¹¹ infectious units, 5.0×10¹¹ infectious units, 1.0×10¹² infectious units, 5.0×10¹² infectious units, or 1.0×10¹³ infectious units, 5.0×10¹³ infectious units, 1.0×10¹⁴ infectious units, 5.0×10¹⁴ infectious units, or 1.0×10¹⁵ infectious units. The techniques available for quantifying infectious units are routine in the art and include viral particle number determination, fluorescence microscopy, and titer by plaque assay. For example, the number of adenovirus particles can be determined by measuring the absorbance at A260. Similarly, infectious units can also be determined by quantitative immunofluorescence of vector specific proteins using monoclonal antibodies or by plaque assay.

In some embodiments, methods that calculate the infectious units include the plaque assay, in which titrations of the virus are grown on cell monolayers and the number of plaques is counted after several days to several weeks. For example, the infectious titer is determined, such as by plaque assay, for example an assay to assess cytopathic effects (CPE). In some embodiments, a CPE assay is performed by serially diluting virus on monolayers of cells, such as HFF cells, that are overlaid with agarose. After incubation for a time period to achieve a cytopathic effect, such as for about 3 to 28 days, generally 7 to 10 days, the cells can be fixed and foci of absent cells visualized as plaques are determined. In some embodiments, infectious units can be determined using an endpoint dilution (TCID50) method, which determines the dilution of virus at which 50% of the cell cultures are infected and hence, generally, can determine the titer within a certain range, such as one log.

In some embodiments, the dosage of administration of a viral vector or virus-like particle is from about 10⁴ to about 10¹⁰ plaque forming units (pfu), inclusive. In some embodiments, the dosage of administration of a viral vector or virus-like particle is from about 10⁹ to about 10¹⁵ pfu, inclusive In some embodiments, the dosage of administration of a viral vector or virus-like particle is from about 10⁵ to about 10⁹ pfu. In some embodiments, the dosage of administration of a viral vector or virus-like particle is from about 10⁶ to about 10⁹ pfu. In some embodiments, the dosage of administration of a viral vector or virus-like particle is from about 10¹² to about 10¹⁴ pfu, inclusive. In some embodiments, the dosage of administration is 1.0×10⁹ pfu, 5.0×10° pfu, 1.0×10¹⁰ pfu, 5.0×10¹⁰ pfu, 1.0×10¹¹ pfu, 5.0×10¹¹ pfu, 1.0×10¹² pfu, 5.0×10¹² pfu, or 1.0×10¹³ pfu, 5.0×10¹³ pfu, 1.0×10¹⁴ pfu, 5.0×10¹⁴ pfu, or 1.0×10¹⁵ pfu.

In some embodiments, the subject will receive a single injection. In some embodiments, administration can be repeated at daily/weekly/monthly intervals for an indefinite period and/or until the efficacy of the treatment has been established. As set forth herein, the efficacy of treatment can be determined by evaluating the symptoms and clinical parameters described herein and/or by detecting a desired response.

The exact amount of vehicle provided lipid particle required will vary from subject to subject, depending on the species, age, weight and general condition of the subject, the particular polynucleic acid, polypeptide, or vector used, its mode of administration etc. TAn appropriate amount can be determined by one of ordinary skill in the art using only routine experimentation given the teachings herein.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the lipid particles in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Injectables can be prepared in conventional forms, either as liquid solutions or suspensions, solid forms suitable for solution of suspension in liquid prior to injection, or as emulsions. As used herein, “parenteral administration” includes intradermal, intranasal, subcutaneous, intramuscular, intraperitoneal, intravenous and intratracheal routes, as well as a slow release or sustained release system such that a constant dosage is maintained.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

Sustained-release preparations may be prepared. Suitable examples of sustained-release preparations include semipermeable matrices of solid hydrophobic polymers containing the antibody, which matrices are in the form of shaped articles, e.g. films, or microcapsules.

In some embodiments, vehicle formulations may comprise cyroprotectants. As used herein, there term “cryoprotectant” refers to one or more agent that when combined with a given substance, helps to reduce or eliminate damage to that substance that occurs upon freezing. In some embodiments, cryoprotectants are combined with vector vehicles in order to stabilize them during freezing. In some aspects, Frozen storage of RNA between −20° C. and −80° C. may be advantageous for long term (e.g. 36 months) stability of polynucleotide. In some embodiments, the RNA species is mRNA. In some embodiments, cryoprotectants are included in vehicle formulations to stabilize polynucleotide through freeze/thaw cycles and under frozen storage conditions. Cryoprotectants of the provided embodiments may include, but are not limited to sucrose, trehalose, lactose, glycerol, dextrose, raffinose and/or mannitol. Trehalose is listed by the Food and Drug Administration as being generally regarded as safe (GRAS) and is commonly used in commercial pharmaceutical formulations.

The formulations to be used for in vivo administration are generally sterile. Sterility may be readily accomplished, e.g., by filtration through sterile filtration membranes.

### IV. Methods Of Use

In some embodiments, the lipid particles provided herein or pharmaceutical compositions containing same can be administered to a subject, e.g. a mammal, e.g. a human. In such embodiments, the subject may be at risk of, may have a symptom of, or may be diagnosed with or identified as having, a particular disease or condition. In one embodiment, the subject has cancer. In one embodiment, the subject has an infectious disease. In some embodiments, the lipid particle contains nucleic acid sequences encoding an exogenous agent for treating the disease or condition in the subject. For example, the exogenous agent is one that targets or is specific for a protein of a neoplastic cells and the lipid particle is administered to a subject for treating a tumor or cancer in the subject. In another example, the exogenous agent is an inflammatory mediator or immune molecule, such as a cytokine, and lipid particle is administered to a subject for treating any condition in which it is desired to modulate (e.g. increase) the immune response, such as a cancer or infectious disease. In some embodiments, the lipid particle is administered in an effective amount or dose to effect treatment of the disease, condition or disorder. Provided herein are uses of any of the provided lipid particles in such methods and treatments, and in the preparation of a medicament in order to carry out such therapeutic methods. In some embodiments, the methods are carried out by administering the lipid particle or compositions comprising the same, to the subject having, having had, or suspected of having the disease or condition or disorder. In some embodiments, the methods thereby treat the disease or condition or disorder in the subject. Also provided herein are uses of any of the compositions, such as pharmaceutical compositions provided herein, for the treatment of a disease, condition or disorder associated with a particular gene or protein targeted by or provided by the exogenous agent.

In some embodiments, the provided methods or uses involve administration of a pharmaceutical composition comprising oral, inhaled, transdermal or parenteral (including intravenous, intratumoral, intraperitoneal, intramuscular, intracavity, intranodal and subcutaneous) administration. In some embodiments, the lipid particle may be administered alone or formulated as a pharmaceutical composition. In some embodiments, the lipid particle or compositions described herein can be administered to a subject, e.g., a mammal, e.g., a human. In some of any embodiments, the subject may be at risk of, may have a symptom of, or may be diagnosed with or identified as having, a particular disease or condition (e.g., a disease or condition described herein). In some embodiments, the disease is a disease or disorder.

In some embodiments, the lipid particles may be administered in the form of a unit-dose composition, such as a unit dose oral, parenteral, transdermal or inhaled composition. In some embodiments, the compositions are prepared by admixture and are adapted for oral, inhaled, transdermal or parenteral administration, and as such may be in the form of tablets, capsules, oral liquid preparations, powders, granules, lozenges, reconstitutable powders, injectable and infusable solutions or suspensions or suppositories or aerosols.

In some embodiments, the regimen of administration may affect what constitutes an effective amount. In some embodiments, the therapeutic formulations may be administered to the subject either prior to or after a diagnosis of disease. In some embodiments, several divided dosages, as well as staggered dosages may be administered daily or sequentially, or the dose may be continuously infused, or may be a bolus injection. In some embodiments, the dosages of the therapeutic formulations may be proportionally increased or decreased as indicated by the exigencies of the therapeutic or prophylactic situation.

In some embodiments, the administration of the compositions of the present invention to a subject, preferably a mammal, more preferably a human, may be carried out using known procedures, at dosages and for periods of time effective to prevent or treat disease. In some embodiments, an effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. In some embodiments, the dosage regimens may be adjusted to provide the optimum therapeutic response. In some embodiments, several divided doses may be administered daily or the dose may be proportionally reduced as indicated by the exigencies of the therapeutic situation. In some embodiments, the effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

In some embodiments, the compound may be administered to a subject as frequently as several times daily, or it may be administered less frequently, such as once a day, once a week, once every two weeks, once a month, or even less frequently, such as once every several months or even once a year or less. In some embodiments, the amount of compound dosed per day may be administered, in non-limiting examples, every day, every other day, every 2 days, every 3 days, every 4 days, or every 5 days. In some embodiments, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

In some embodiments, dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. In some embodiments, the physician or veterinarian can start doses of the compounds of the invention employed in the pharmaceutical composition at levels lower than that required in order to achieve the desired therapeutic effect and gradually increase the dosage until the desired effect is achieved.

In some embodiments, it is especially advantageous to formulate the compound in dosage unit form for ease of administration and uniformity of dosage. In some embodiments, dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. In some embodiments, the dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In some embodiments, the term “container” includes any receptacle for holding the pharmaceutical composition. In some embodiments, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. It should be understood that the instructions for use of the pharmaceutical composition may be contained on the packaging containing the pharmaceutical composition, and as such the instructions form an increased functional relationship to the packaged product. In some embodiments, instructions may contain information pertaining to the compound's ability to perform its intended function, e.g., treating or preventing a disease in a subject, or delivering an imaging or diagnostic agent to a subject.

In some embodiments, routes of administration of any of the compositions disclosed herein include oral, nasal, rectal, parenteral, sublingual, transdermal, transmucosal (e.g., sublingual, lingual, (trans) buccal, (trans) urethral, vaginal (e.g., trans- and perivaginally), (intra) nasal, and (trans) rectal), intravesical, intrapulmonary, intraduodenal, intragastrical, intrathecal, subcutaneous, intramuscular, intradermal, intra-arterial, intravenous, intrabronchial, inhalation, and topical administration.

In some of any embodiments, suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like.

In some embodiments, the lipid particle composition comprising an exogenous agent or cargo, may be used to deliver such exogenous agent or cargo to a cell tissue or subject. In some embodiments, delivery of a cargo by administration of a lipid particle composition described herein may modify cellular protein expression levels. In certain embodiments, the administered composition directs upregulation of (via expression in the cell, delivery in the cell, or induction within the cell) of one or more cargo (e.g., a polypeptide or mRNA) that provide a functional activity which is substantially absent or reduced in the cell in which the polypeptide is delivered. In some embodiments, the missing functional activity may be enzymatic, structural, or regulatory in nature. In some embodiments, the administered composition directs up-regulation of one or more polypeptides that increases (e.g., synergistically) a functional activity which is present but substantially deficient in the cell in which the polypeptide is upregulated. In some of any embodiments, the administered composition directs downregulation of (via expression in the cell, delivery in the cell, or induction within the cell) of one or more cargo (e.g., a polypeptide, siRNA, or miRNA) that repress a functional activity which is present or upregulated in the cell in which the polypeptide, siRNA, or miRNA is delivered. In some of any embodiments, the upregulated functional activity may be enzymatic, structural, or regulatory in nature. In some embodiments, the administered composition directs down-regulation of one or more polypeptides that decreases (e.g., synergistically) a functional activity which is present or upregulated in the cell in which the polypeptide is downregulated. In some embodiments, the administered composition directs upregulation of certain functional activities and downregulation of other functional activities.

In some of any embodiments, the lipid particle composition (e.g., one comprising mitochondria or DNA) mediates an effect on a target cell, and the effect lasts for at least 1, 2, 3, 4, 5, 6, or 7 days, 2, 3, or 4 weeks, or 1, 2, 3, 6, or 12 months. In some embodiments (e.g., wherein the lipid particle composition comprises an exogenous protein), the effect lasts for less than 1, 2, 3, 4, 5, 6, or 7 days, 2, 3, or 4 weeks, or 1, 2, 3, 6, or 12 months.

In some of any embodiments, the lipid particle composition described herein is delivered ex-vivo to a cell or tissue, e.g., a human cell or tissue. In embodiments, the composition improves function of a cell or tissue ex-vivo, e.g., improves cell viability, respiration, or other function (e.g., another function described herein).

In some embodiments, the composition is delivered to an ex vivo tissue that is in an injured state (e.g., from trauma, disease, hypoxia, ischemia or other damage).

In some embodiments, the composition is delivered to an ex-vivo transplant (e.g., a tissue explant or tissue for transplantation, e.g., a human vein, a musculoskeletal graft such as bone or tendon, cornea, skin, heart valves, nerves; or an isolated or cultured organ, e.g., an organ to be transplanted into a human, e.g., a human heart, liver, lung, kidney, pancreas, intestine, thymus, eye). In some embodiments, the composition is delivered to the tissue or organ before, during and/or after transplantation.

In some embodiments, the composition is delivered, administered or contacted with a cell, e.g., a cell preparation. In some embodiments, the cell preparation may be a cell therapy preparation (a cell preparation intended for administration to a human subject). In embodiments, the cell preparation comprises cells expressing a chimeric antigen receptor (CAR), e.g., expressing a recombinant CAR. The cells expressing the CAR may be, e.g., T cells, Natural Killer (NK) cells, cytotoxic T lymphocytes (CTL), regulatory T cells. In embodiments, the cell preparation is a neural stem cell preparation. In embodiments, the cell preparation is a mesenchymal stem cell (MSC) preparation. In embodiments, the cell preparation is a hematopoietic stem cell (HSC) preparation. In embodiments, the cell preparation is an islet cell preparation.

In some embodiments, the lipid particle compositions described herein can be administered to a subject, e.g., a mammal, e.g., a human. In such embodiments, the subject may be at risk of, may have a symptom of, or may be diagnosed with or identified as having, a particular disease or condition (e.g., a disease or condition described herein).

In some embodiments, the source of lipid particles are from the same subject that is administered a lipid particle composition. In other embodiments, they are different. In some embodiments, the source of lipid particles and recipient tissue may be autologous (from the same subject) or heterologous (from different subjects). In some embodiments, the donor tissue for lipid particle compositions described herein may be a different tissue type than the recipient tissue. In some embodiments, the donor tissue may be muscular tissue and the recipient tissue may be connective tissue (e.g., adipose tissue). In other embodiments, the donor tissue and recipient tissue may be of the same or different type, but from different organ systems.

In some embodiments, the lipid particle composition described herein may be administered to a subject having a cancer, an autoimmune disease, an infectious disease, a metabolic disease, a neurodegenerative disease, or a genetic disease (e.g., enzyme deficiency). In some embodiments, the subject is in need of regeneration.

In some embodiments, the lipid particle is co-administered with an inhibitor of a protein that inhibits membrane fusion. For example, Suppressyn is a human protein that inhibits cell-cell fusion (Sugimoto et al., “A novel human endogenous retroviral protein inhibits cell-cell fusion” Scientific Reports 3:1462 (DOI: 10.1038/srep01462)). In some embodiments, the lipid particle particles is co-administered with an inhibitor of sypressyn, e.g., a siRNA or inhibitory antibody.

In some embodiments, also provided herein are methods of mobilizing hematopoietic cells, such as hematopoietic stem cells (HSCs), in connection with the provided methods of delivering a particle (e.g., lentiviral particle), including a particle containing an exogenous agent for a target cell. In some embodiments, the target cell targeted for delivery by a provided particle (e.g., lentiviral particle) is a hematopoietic stem cell (HSC). In some embodiments, the method of delivery includes administering a mobilization agent, e.g., a mobilization regimen, to the subject and administering the particle (e.g., lentiviral particle) to the subject in accordance with the provided methods. In some embodiments, the mobilization agent is administered to the subject prior to introducing or administering the particles (e.g., lentiviral particle) to the subject. In some embodiments, the mobilization agent includes a mobilization regimen that cause therapeutically inaccessible hematopoietic cells to become therapeutically accessible. In some embodiments, a mobilization agent increases the number of hematopoietic cells in peripheral blood, thus allowing for a more accessible source of hematopoietic cells for targeting by the particles (e.g., lentiviral particles) in accordance with the methods as described. In some embodiments, the mobilization agent stimulates mobilization of bone marrow cells from the bone marrow to the peripheral blood in the subject.

Also provided herein in some embodiments are methods involving administering to a subject (i) an agent that stimulates mobilization of bone marrow cells from the bone marrow to the peripheral blood in the subject and (ii) a particle, such as any described in Section II, either with or without any of the targeting agents described herein.

In some embodiments, the mobilization agent increases the number of stem cells in peripheral blood, thus allowing for a more accessible source of stem cells for use in the method as described. In some embodiments, the mobilization agent increases the number of hematopoietic cells in circulation in the subject. In some embodiments, the mobilization agent is a mobilizer of hematopoietic stem cells or progenitor cells. In some embodiments, the mobilization agent induces the exit of hematopoietic cells from bone marrow.

In some embodiments, the hematopoietic cells are CD34+ and may include CD34+ progenitor cells. In some embodiments, the hematopoietic cells are HSCs. In some aspects, the mobilization agent is a stem cell mobilization agent.

As used herein, “mobilizing” and “mobilizing hematopoietic cells” are used interchangeably to refer to the act of inducing the migration of hematopoietic cells, such as CD34+ cells, including progenitor cells and/or hematopoietic stem cells from a first location (e.g., stem cell niche, e.g., bone marrow) into a second location (e.g., tissue (e.g., peripheral blood) or organ (e.g., spleen). In some embodiments, the process of mobilizing hematopoietic cells involves the recruitment of stem cells from their tissue or organ of residence to peripheral blood following treatment with a mobilization agent, such as using a mobilization agent known to a skilled artisan, including any cytokine and chemotherapeutic drugs known in the art for this purpose (e.g., G-CSF). In some aspects, this process mimics the enhancement of the physiological release of stem cells from tissues or organs in response to stress signals during injury and inflammation. In some embodiments, the mobilization agent or agents act as agonists or antagonists that prevent the attachment of hematopoietic cells to cells or tissues of their microenvironment. In some embodiments, the mobilization agent or agents induce the release of proteases that cleave the adhesion molecules or support structures between hematopoietic cells and their sites of attachment. In some embodiments, the mobilization agent is capable of mobilizing any hematopoietic cell, such as stem cells and/or progenitor cells, in which heparan sulfate proteoglycans are responsible for maintaining adhesion of the cells in their cell niche. In an aspect, a method of mobilizing hematopoietic cells in a subject comprises administering to a subject an effective amount of an agent that inhibits the level or activity of heparan sulfate proteoglycans, thereby mobilizing hematopoietic cells in the subject.

In some embodiments, a mobilization agent increases the circulation of hematopoietic cells and/or mobilizes hematopoietic cells sequestered in bone marrow to exit bone marrow into compartments where they are accessible, e.g., accessible for transduction by the lipid particle and/or viral vector. For example, administration to a subject of a mobilization therapy can increase the circulation of hematopoietic cells and/or mobilize hematopoietic cells sequestered in bone marrow to exit bone marrow into compartments where they are accessible, such as the peripheral blood.

In some embodiments, the mobilization agent is administered prior to the particle (e.g., lentiviral particle or other viral particle). In some embodiments, the mobilization agent is administered to the subject within 7 days prior to administering the particle (e.g., lentiviral particle), such as within 6 days, 5 days, 4 days, 3 days, 2 days or 1 day prior to administering the particle (e.g., lentiviral particle). In some embodiments, the mobilization agent is administered twice a day, once a day or two or three times within a week. In some embodiments, the mobilization agent is administered once daily for consecutive days prior to administering the particle (e.g., lentiviral particle). In some embodiments, at least one dose of the mobilization agent is administered to the subject on the same day as the provided methods of contacting target cells with a particle (e.g., lentiviral particle). In some embodiments, at least one dose of the mobilization agent is administered to the subject within 12 hours prior to administering the lipid particle (e.g. lentiviral vector), such as within 10 hours, 8 hours, 6 hours, 5 hours, 4 hours, 3 hours, 2 hours or 1 hour prior to administering the particle (e.g., lentiviral particle).

Exemplary mobilization agents include stem cell factor (SCF), small molecule VLA-4 inhibitor BI05192, BOP (N-(benzenesulfonyl)-L-prolyl-L-0-(1-pyrrolidinylcarbonyl) tyrosine), heparin, granulocyte colony-stimulating factor (G-CSF), and plerixafor (also known as AMD3100). In some embodiments, the mobilization agent of the method is selected from the group consisting of granulocyte-macrophage colony-stimulating factor (GM-CSF), Fms-related tyrosine kinase 3 (flt-3) ligand, stromal cell-derived factor 1 (SDF-1), agonists of the chemokine (C—C motif) receptor 1 (CCR1), such as chemokine (C—C motif) ligand 3 (CCL3, also known as macrophage inflammatory protein-1α (Mip-1α)), agonists of the chemokine (C—X—C motif) receptor 1 (CXCR1) and CXCR2, such as chemokine (C—X—C motif) ligand (CXCL1), CXCL2 (also known as growth-related oncogene protein-β (Gro-β)), and CXCL8 (also known as interleukin-8 (IL-8)), agonists of CXCR4, such as CTCE-002, ATI-2341, and Met-SDF-1, Very Late Antigen (VLA)-4 inhibitor, TG-0054, AMD3465, and any combination thereof.

In some embodiments, the mobilization agent is stem cell factor (SCF), small molecule VLA-4 inhibitor BI05192, BOP (N-(benzenesulfonyl)-L-prolyl-L-0-(1-pyrrolidinylcarbonyl) tyrosine), heparin, granulocyte colony-stimulating factor (G-CSF), or plerixafor (AMD3100). In some embodiments, the mobilization agent includes the combination of G-CSF and plerixafor (AMD3100). In some of any embodiments, G-CSF is filgrastim (e.g. Neupogen® or Zarzio®). In some embodiments, the G-CSF is a pegylated G-CSF, such as pegfilgrastim (e.g. Neulasta®).

Any of various known methods for mobilizing hematopoietic cells using a mobilization agent can be used, including but not limited to, any as described in PCT publication No. WO2021211450; U.S. publication Nos. US20200268850 and US20170106021; and U.S. Pat. Nos. 7,939,057 and 10,907,177.

In some embodiments, the mobilization regimen includes administration of at least one mobilizing agent. In various embodiments, at least one mobilizing agent is administered to a subject (i) daily on the four days prior to administration of a first dose of the particle (e.g., lentiviral particle); (ii) on the day of administration of a first dose of the particle (e.g., lentiviral particle); and (iii) on the day of administration of one or more subsequent doses of the particle (e.g., lentiviral particle). In some embodiments, at least one mobilizing agent is administered to a subject (i) on the day prior to administration of a first dose of the particle (e.g., lentiviral particle) and (ii) on the day of administration of a first dose of the particle (e.g., lentiviral particle).

In some embodiments, the mobilization regimen includes administration of one or both of G-CSF and plerixafor/AMD3100. In various embodiments G-CSF is administered to a subject (i) daily on the four days prior to administration of a first dose of the particle (e.g., lentiviral particle); (ii) on the day of administration of a first dose of the particle (e.g., lentiviral particle); and (iii) on the day of administration of one or more subsequent doses of the particle (e.g., lentiviral particle). In various embodiments plerixafor/AMD3100 is administered to a subject (i) on the day prior to administration of a first dose of the particle (e.g., lentiviral particle) and (ii) on the day of administration of a first dose of the particle (e.g., lentiviral particle).

In some embodiments, the at least one mobilizing agent is administered once daily at a dose that is, or is at least, 0.1, 1.0, 10, 20, 30, 40, 50, 75, 100, 150, or 200 μg/kg. In various embodiments, a daily dose of the at least one mobilizing agent has a range having a lower bound of 0.1 μg/kg/day, 1.0 μg/kg/day, 10 μg/kg/day, 20 μg/kg/day, 30 μg/kg/day, 40 μg/kg/day, 50 μg/kg/day, or 75 μg/kg/day and an upper bound of 100 μg/kg/day, 150 μg/kg/day, or 200 μg/kg/day. In various embodiments the at least one mobilizing agent is administered once daily at a dose that is, or is at least, 1 mg/kg, 2 mg/kg, 3 mg/kg, 4 mg/kg, 5 mg/kg, 7.5 mg/kg, 10 mg/kg, 15 mg/kg, or 20 mg/kg. In various embodiments, a daily dose of the at least one stem cell mobilizing agent has a range having a lower bound of 1 mg/kg/day, 2 mg/kg/day, 3 mg/kg/day, 4 mg/kg/day, 5 mg/kg/day, or 7.5 mg/kg/day and an upper bound of 10 mg/kg/day, 15 mg/kg/day, or 20 mg/kg/day.

In various embodiments G-CSF is administered once daily at a dose that is, or is at least, 10 μg/kg, 20 μg/kg, 30 μg/kg, 40 μg/kg, 50 μg/kg, 75 μg/kg, 100 μg/kg, 150 μg/kg, or 200 μg/kg. In various embodiments, a daily dose of G-CSF has a range having a lower bound of 10 μg/kg/day, 20 μg/kg/day, 30 μg/kg/day, 40 μg/kg/day, 50 μg/kg/day, or 75 μg/kg/day and an upper bound of 100 μg/kg/day, 150 μg/kg/day, or 200 μg/kg/day. In various embodiments plerixafor/AMD3100 is administered once daily at a dose that is, or is at least, 1 mg/kg, 2 mg/kg, 3 mg/kg, 4 mg/kg, 5 mg/kg, 7.5 mg/kg, 10 mg/kg, 15 mg/kg, or 20 mg/kg. In various embodiments, a daily dose of G-CSF has a range having a lower bound of 1 mg/kg/day, 2 mg/kg/day, 3 mg/kg/day, 4 mg/kg/day, 5 mg/kg/day, or 7.5 mg/kg/day and an upper bound of 10 mg/kg/day, 15 mg/kg/day, or 20 mg/kg/day.

In some embodiments, G-CSF may be administered daily as a dose of 0.5-16 μg/kg (e.g., 5-16 μg/kg or 10-16 μg/kg) for 1-10 days (e.g., 1-7 days, or particularly, 1-3 days). In another example, G-CSF may be administered to a healthy donor at a dosage of 10-16 μg/kg daily for up to seven days. Three or four days of treatment may be sufficient when the peripheral blood collections are combined with apheresis starting on, e.g., day 4. In another example, G-CSF may be administered at a dosage of 10 μg/kg daily for four days with apheresis starting on, e.g., day 4. The most commonly used dosage of G-CSF in healthy donors is 10 μg/kg body weight daily with leukapheresis starting on day 5 onward until collection of an adequate number of stem cells (e.g., collections can be taken once or twice daily for 1 to 4 days, such as 1 or 2 days). G-CSF may be administered subcutaneously or intravenously. Methods of G-CSF administration and dosage are described in Juttner et al. (Blood 89:2233-2258, 1997), Kroschinsky et al. (Haematologica 90:1556-1671, 2005), U.S. Pat. No. 6,162,427, 2005/0186182, WO 2010051335, and WO 2005014023, all of which are incorporated herein by reference in their entireties. If cyclophosphamide is administered for mobilization, G-CSF is usually started 2-5 days after completion of cyclophosphamide infusion. Methods of administering combined mobilization agents of G-CSF and one or more chemotherapeutic agents are described in Andre et al. (Transfusion 43:50-57, 2003), Ataergin et al. (Am. J. Hematol. 83:644-648, 2008), and Demirer et al. (Br. J. Haematol. 116:468-474, 2002), all of which are incorporated herein by reference in their entireties.

Plerixafor may be administered at a dosage of 1-300 μg/kg. At 240 μg/kg, the number of mobilized stem cells peak at around 4-10 hours after plerixafor administration. In some examples, plerixafor may be administered once or twice daily at a dose of ~1-300 μg/kg (e.g., 100-300 μg/kg or 200-300 μg/kg) for 1-10 days (e.g., 1-5 days).

The combination of plerixafor and G-CSF for stem cell mobilization was approved by the FDA in 2008 for use in patients with non-Hodgkin's lymphoma and multiple myeloma. This combination therapy may also be used to mobilize stem cells. A typical combination therapy may include, e.g., the administration of G-CSF at ^{~}0.5-16 μg/kg (e.g., 10 μg/kg) daily with plerixafor at 1-300 μg/kg (e.g., 240 μg/kg) given a few days (e.g., 1-3 days) after G-CSF administration. Both agents may be given together for about 2-10 days (e.g., 4 days) or until adequate hematopoietic cells are collected. Plerixafor, either alone or in combination with G-CSF, may be administered subcutaneously or intravenously.

In some embodiments, the method of mobilizing hematopoietic cells in a subject includes administering to the subject at least one mobilization agent. In some embodiments, the method of mobilizing hematopoietic cells includes administering to the subject at least one mobilization agent comprising (i) at least one heparan sulfate inhibitor and (ii) at least one of a CXCR2 agonist and a CXCR4 antagonist.

In some embodiments, the mobilized hematopoietic cells comprise KLS-CD150+CD48-cells. In some embodiments, the mobilized hematopoietic cells comprise CD34-CD133+ cells. In some embodiments, the mobilized hematopoietic cells and/or progenitor cells comprise common myeloid progenitor cells. In some embodiments, the mobilized hematopoietic cells and/or progenitor cells comprise granulocyte/monocyte progenitor cells. In some embodiments, the mobilized hematopoietic cells and/or progenitor cells comprise megakaryocyte/erythroid progenitor cells. In some embodiments, the mobilized hematopoietic cells and/or progenitor cells comprise committed lymphoid progenitor cells. In some embodiments, the mobilized hematopoietic cells and/or progenitor cells comprise a combination of common myeloid progenitor cells, granulocyte/monocyte progenitor cells, megakaryocyte/erythroid progenitor cells. In some embodiments, the hematopoietic progenitor cells comprise CD150-CD48-CD244+ cells. In some embodiments, the hematopoietic progenitor cells comprise CD150-CD48+CD244+ cells. In some embodiments, the hematopoietic progenitor cells comprise Lin-SCA-1-c-Kit+CD34+CD16/32mid cells. In some embodiments, the hematopoietic progenitor cells comprise lin-SCA-1-c-kit+CD34-CD16/32low cells. In some embodiments, the mobilized hematopoietic cells comprise CD34+ peripheral blood stem cells.

### V. EXEMPLARY EMBODIMENTS

### Exemplary Embodiments A

1. A lipid particle, comprising: (a) a retargeted attachment protein comprising a paramyxovirus envelope attachment protein linked to (i) a first targeting moiety directed to a first target molecule expressed on the surface of a target cell, and (ii) a second targeting moiety directed to a second target molecule expressed on the surface of a target cell; and (b) at least one paramyxovirus fusion (F) protein; and wherein the protein in (a), and (b) are exposed on the outside of the lipid bilayer.
2. The lipid particle of embodiment 1, wherein the paramyxovirus attachment protein is a variant paramyxovirus envelope attachment protein comprising one or more mutations to reduce native tropism relative to the wild-type paramyxovirus envelope attachment protein not comprising the one or more mutations.
3. The lipid particle of embodiment 1 or 2, wherein the paramyxovirus envelope attachment protein is a first paramyxovirus envelope attachment protein and the lipid particle further comprises a second paramyxovirus envelope attachment protein, wherein the second paramyxovirus envelope attachment protein is a variant paramyxovirus envelope attachment protein comprising one or more mutations to reduce native tropism relative to the wild-type paramyxovirus envelope attachment protein not comprising the one or more mutations, wherein the second paramyxovirus envelope attachment protein is exposed on the outside of the lipid bilayer.
4. The lipid particle of any one of embodiments 1-3, wherein the targeting one or both of the first target molecule and the second target molecule does not activate or inhibit, induce a phenotype change (for example maturation and/or differentiation), induce proliferation, and/or induce apoptosis of said target cell.
5. A lipid particle, comprising: (a) a retargeted attachment protein comprising a first paramyxovirus envelope attachment protein operably linked to (i) a first targeting moiety directed to a first target molecule expressed on the surface of a target cell, and (ii) a second targeting moiety directed to a second target molecule expressed on the surface of a target cell, (b) a second paramyxovirus envelope attachment protein that is a variant paramyxovirus envelope attachment protein comprising one or more mutations to reduce the native tropism relative to the wild-type paramyxovirus envelope attachment protein not comprising the one or more mutations; and (c) at least one paramyxovirus fusion (F) protein; and wherein the protein in (a), (b) and (c) are exposed on the outside of the lipid bilayer.
6. The lipid particle of embodiments 1-5, wherein the first paramyxovirus envelope attachment protein is a variant paramyxovirus envelope attachment protein.
7. The lipid particle of embodiment 6, wherein the variant paramyxovirus envelope attachment protein comprises one or more mutations that reduces native tropism relative to the wild-type paramyxovirus envelope attachment protein not comprising the one or more mutations.
8. The lipid particle of any of embodiments 1-8, wherein the first and second paramyxovirus envelope attachment protein are the same.
9. The lipid particle of any of embodiments 1-8, wherein the second paramyxovirus envelope attachment protein is not linked or fused to a non-viral heterologous moiety that is a cell-specific targeting domain or functional domain.
10. The lipid particle of embodiments 1-9, wherein the first and second targeting moiety are each not selected from the group consisting of a cytokine, growth factor, hormone, neurotransmitter, apoptosis ligand and their combinations.
11. The lipid particle of any of embodiments 1-10, wherein targeting one or both of the first target molecule and the second target molecule does not modulate or induce a signal in the target cell.
12. The lipid particle of any of embodiments 1-11, wherein the first and second targeting moieties each bind to a cell surface molecule present on a target cell.
13. The lipid particle of any of embodiments 1-11, wherein the first targeting moiety binds to a cell surface molecule present on a first target cell, and the second targeting moiety binds a surface molecule present on a second target cell.
14. The lipid particle of any of embodiments 1-13, wherein the first and second target molecule are different target molecules.
15. The lipid particle of any of embodiments 1-14, wherein the first and second target molecule are the same target molecule.
16. The lipid particle of embodiment 15, wherein the first and second targeting moiety bind distinct epitopes of the same target molecule.
17. The lipid particle of any of embodiments 1-16, wherein the cell surface molecule is a protein, glycan, or lipid.
18. The lipid particle of any of embodiments 1-17, wherein the target cell is selected from the group consisting of tumor-infiltrating lymphocytes, T cells, neoplastic or tumor cells, virus-infected cells, stem cells, central nervous system (CNS) cells, hematopoietic stem cells (HSCs), and liver cells.
19. The lipid particle of any of embodiments 1-18, wherein the target cells is selected from the group consisting of a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+B cell, a CD19+B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.
20. The lipid particle of any of embodiments 1-19, wherein the target cell is a hepatocyte.
21. The lipid particle of embodiment 20, wherein the cell surface molecule is selected from the group consisting of CD34, CD117, and CD133.
22. The lipid particle of any of embodiments 1-19, wherein the target cell is a T cell.
23. The lipid particle of embodiment 22, wherein the cell surface molecule is selected from the group consisting of CD3, CD4, CD7 CD8, ASCT2, CD105, CD110, CD117, CD133, CD146, CD164, CD34, CD46, CD49f, CD90, EPCR, and ITGA3.
24. A lipid particle, comprising: (a) a first retargeted attachment protein comprising (i) a first paramyxovirus envelope attachment protein; and (ii) a first targeting moiety directed to CD133; and a second targeting moiety directed to CD133; and (b) at least one paramyxovirus fusion (F) protein; and the protein in (a), and (b) are exposed on the outside of the lipid bilayer.
25. The lipid particle of any of embodiments 1-24, wherein: (i) the first targeting moiety is an scFv and comprises an amino acid sequence selected from the group consisting of SEQ ID NOs: 516, 525, 534, 543, and 552, or an amino acid sequence having at least 90%, 91%, 92%, 93%, 94%, 95%, 96%, 97%, 98%, or 99% sequence identity thereto; and/or (ii) the second targeting moiety is an scFv and comprises an amino acid sequence selected from the group consisting of SEQ ID NOs: 516, 525, 534, 543, and 552, or an amino acid sequence having at least 90%, 91%, 92%, 93%, 94%, 95%, 96%, 97%, 98%, or 99% sequence identity thereto.
26. The lipid particle of any of embodiments 1-24, wherein: (i) the first targeting moiety comprises: (a) a CD133 binding agent comprising a CDR-H1, a CDR-H2, and a CDR-H3 comprising the amino acid sequences of SEQ ID NOs: 536, 537, and 538, respectively, and a CDR-L1, a CDR-L2, and a CDR-L3 comprising the amino acid sequences of SEQ ID NOs: 540, 541, and 542, respectively; (b) a CD133 binding agent comprising a CDR-H1, a CDR-H2, and a CDR-H3 comprising the amino acid sequences of SEQ ID NOs: 545, 546, and 547, respectively, and a CDR-L1, a CDR-L2, and a CDR-L3 comprising the amino acid sequences of SEQ ID NOs: 549, 550, and 551, respectively; (c) a CD133 binding agent comprising a CDR-H1, a CDR-H2, and a CDR-H3 comprising the amino acid sequences of SEQ ID NOs: 518, 519, and 520, respectively, and a CDR-L1, a CDR-L2, and a CDR-L3 comprising the amino acid sequences of SEQ ID NOs: 522, 523, and 524, respectively; (d) a CD133 binding agent comprising a CDR-H1, a CDR-H2, and a CDR-H3 comprising the amino acid sequences of SEQ ID NOs: 527, 528, and 529, respectively, and a CDR-L1, a CDR-L2, and a CDR-L3 comprising the amino acid sequences of SEQ ID NOs: 531, 532, and 533, respectively; or (e) a CD133 binding agent comprising a CDR-H1, a CDR-H2, and a CDR-H3 comprising the amino acid sequences of SEQ ID NOs: 554, 555, and 556, respectively, and a CDR-L1, a CDR-L2, and a CDR-L3 comprising the amino acid sequences of SEQ ID NOs: 558, 559, and 560, respectively; and/or (ii) the second targeting moiety comprises: (a) a CD133 binding agent comprising a CDR-H1, a CDR-H2, and a CDR-H3 comprising the amino acid sequences of SEQ ID NOs: 536, 537, and 538, respectively, and a CDR-L1, a CDR-L2, and a CDR-L3 comprising the amino acid sequences of SEQ ID NOs: 540, 541, and 542, respectively; (b) a CD133 binding agent comprising a CDR-H1, a CDR-H2, and a CDR-H3 comprising the amino acid sequences of SEQ ID NOs: 545, 546, and 547, respectively, and a CDR-L1, a CDR-L2, and a CDR-L3 comprising the amino acid sequences of SEQ ID NOs: 549, 550, and 551, respectively; (c) a CD133 binding agent comprising a CDR-H1, a CDR-H2, and a CDR-H3 comprising the amino acid sequences of SEQ ID NOs: 518, 519, and 520, respectively, and a CDR-L1, a CDR-L2, and a CDR-L3 comprising the amino acid sequences of SEQ ID NOs: 522, 523, and 524, respectively; (d) a CD133 binding agent comprising a CDR-H1, a CDR-H2, and a CDR-H3 comprising the amino acid sequences of SEQ ID NOs: 527, 528, and 529, respectively, and a CDR-L1, a CDR-L2, and a CDR-L3 comprising the amino acid sequences of SEQ ID NOs: 531, 532, and 533, respectively; or (e) a CD133 binding agent comprising a CDR-H1, a CDR-H2, and a CDR-H3 comprising the amino acid sequences of SEQ ID NOs: 554, 555, and 556, respectively, and a CDR-L1, a CDR-L2, and a CDR-L3 comprising the amino acid sequences of SEQ ID NOs: 558, 559, and 560, respectively.
27. The lipid particle of any of embodiments 1-24, wherein: (i) the first targeting moiety comprises: (a) a CD133 binding agent comprising a CDR-H1, a CDR-H2, and a CDR-H3 comprising the amino acid sequences of SEQ ID NOs: 289, 565, and 538, respectively, and a CDR-L1, a CDR-L2, and a CDR-L3 comprising the amino acid sequences of SEQ ID NOs: 540, 541, and 542, respectively; (b) a CD133 binding agent comprising a CDR-H1, a CDR-H2, and a CDR-H3 comprising the amino acid sequences of SEQ ID NOs: 566, 567, and 547, respectively, and a CDR-L1, a CDR-L2, and a CDR-L3 comprising the amino acid sequences of SEQ ID NOs: 549, 550, and 551, respectively; (c) a CD133 binding agent comprising a CDR-H1, a CDR-H2, and a CDR-H3 comprising the amino acid sequences of SEQ ID NOs: 568, 569, and 520, respectively, and a CDR-L1, a CDR-L2, and a CDR-L3 comprising the amino acid sequences of SEQ ID NOs: 522, 523, and 524, respectively; (d) a CD133 binding agent comprising a CDR-H1, a CDR-H2, and a CDR-H3 comprising the amino acid sequences of SEQ ID NOs: 570, 571, and 529, respectively, and a CDR-L1, a CDR-L2, and a CDR-L3 comprising the amino acid sequences of SEQ ID NOs: 531, 532, and 533, respectively; or (e) a CD133 binding agent comprising a CDR-H1, a CDR-H2, and a CDR-H3 comprising the amino acid sequences of SEQ ID NOs: 572, 573, and 556, respectively, and a CDR-L1, a CDR-L2, and a CDR-L3 comprising the amino acid sequences of SEQ ID NOs: 558, 559, and 560, respectively; and/or (ii) the second targeting moiety comprises: (a) a CD133 binding agent comprising a CDR-H1, a CDR-H2, and a CDR-H3 comprising the amino acid sequences of SEQ ID NOs: 289, 565, and 538, respectively, and a CDR-L1, a CDR-L2, and a CDR-L3 comprising the amino acid sequences of SEQ ID NOs: 540, 541, and 542, respectively; (b) a CD133 binding agent comprising a CDR-H1, a CDR-H2, and a CDR-H3 comprising the amino acid sequences of SEQ ID NOs: 566, 567, and 547, respectively, and a CDR-L1, a CDR-L2, and a CDR-L3 comprising the amino acid sequences of SEQ ID NOs: 549, 550, and 551, respectively; (c) a CD133 binding agent comprising a CDR-H1, a CDR-H2, and a CDR-H3 comprising the amino acid sequences of SEQ ID NOs: 568, 569, and 520, respectively, and a CDR-L1, a CDR-L2, and a CDR-L3 comprising the amino acid sequences of SEQ ID NOs: 522, 523, and 524, respectively; (d) a CD133 binding agent comprising a CDR-H1, a CDR-H2, and a CDR-H3 comprising the amino acid sequences of SEQ ID NOs: 570, 571, and 529, respectively, and a CDR-L1, a CDR-L2, and a CDR-L3 comprising the amino acid sequences of SEQ ID NOs: 531, 532, and 533, respectively; or (e) a CD133 binding agent comprising a CDR-H1, a CDR-H2, and a CDR-H3 comprising the amino acid sequences of SEQ ID NOs: 572, 573, and 556, respectively, and a CDR-L1, a CDR-L2, and a CDR-L3 comprising the amino acid sequences of SEQ ID NOs: 558, 559, and 560, respectively.
28. The lipid particle of any of embodiments 1-24, wherein: (i) the first targeting moiety comprises: (a) a CD133 binding agent comprising a heavy chain variable (VH) region comprising the amino acid sequence of SEQ ID NO: 535, or an amino acid sequence having at least 90%, 91%, 92%, 93%, 94%, 95%, 96%, 97%, 98%, or 99% sequence identity thereto; and a light chain variable (VL) region comprising the amino acid sequence of SEQ ID NO: 539, or an amino acid sequence having at least 90%, 91%, 92%, 93%, 94%, 95%, 96%, 97%, 98%, or 99% sequence identity thereto; (b) a CD133 binding agent comprising a VH region comprising the amino acid sequence of SEQ ID NO: 544, or an amino acid sequence having at least 90%, 91%, 92%, 93%, 94%, 95%, 96%, 97%, 98%, or 99% sequence identity thereto; and a VL region comprising the amino acid sequence of SEQ ID NO: 548, or an amino acid sequence having at least 90%, 91%, 92%, 93%, 94%, 95%, 96%, 97%, 98%, or 99% sequence identity thereto; (c) a CD133 binding agent comprising a VH region comprising the amino acid sequence of SEQ ID NO: 517, or an amino acid sequence having at least 90%, 91%, 92%, 93%, 94%, 95%, 96%, 97%, 98%, or 99% sequence identity thereto; and a VL region comprising the amino acid sequence of SEQ ID NO: 521, or an amino acid sequence having at least 90%, 91%, 92%, 93%, 94%, 95%, 96%, 97%, 98%, or 99% sequence identity thereto; (d) a CD133 binding agent comprising a VH region comprising the amino acid sequence of SEQ ID NO: 526, or an amino acid sequence having at least 90%, 91%, 92%, 93%, 94%, 95%, 96%, 97%, 98%, or 99% sequence identity thereto; and a VL region comprising the amino acid sequence of SEQ ID NO: 530, or an amino acid sequence having at least 90%, 91%, 92%, 93%, 94%, 95%, 96%, 97%, 98%, or 99% sequence identity thereto; or (e) a CD133 binding agent comprising a VH region comprising the amino acid sequence of SEQ ID NO: 553, or an amino acid sequence having at least 90%, 91%, 92%, 93%, 94%, 95%, 96%, 97%, 98%, or 99% sequence identity thereto; and a VL region comprising the amino acid sequence of SEQ ID NO: 557, or an amino acid sequence having at least 90%, 91%, 92%, 93%, 94%, 95%, 96%, 97%, 98%, or 99% sequence identity thereto; and/or (ii) the second targeting moiety comprises: (a) a CD133 binding agent comprising a heavy chain variable (VH) region comprising the amino acid sequence of SEQ ID NO: 535, or an amino acid sequence having at least 90%, 91%, 92%, 93%, 94%, 95%, 96%, 97%, 98%, or 99% sequence identity thereto; and a light chain variable (VL) region comprising the amino acid sequence of SEQ ID NO: 539, or an amino acid sequence having at least 90%, 91%, 92%, 93%, 94%, 95%, 96%, 97%, 98%, or 99% sequence identity thereto; (b) a CD133 binding agent comprising a VH region comprising the amino acid sequence of SEQ ID NO: 544, or an amino acid sequence having at least 90%, 91%, 92%, 93%, 94%, 95%, 96%, 97%, 98%, or 99% sequence identity thereto; and a VL region comprising the amino acid sequence of SEQ ID NO: 548, or an amino acid sequence having at least 90%, 91%, 92%, 93%, 94%, 95%, 96%, 97%, 98%, or 99% sequence identity thereto; (c) a CD133 binding agent comprising a VH region comprising the amino acid sequence of SEQ ID NO: 517, or an amino acid sequence having at least 90%, 91%, 92%, 93%, 94%, 95%, 96%, 97%, 98%, or 99% sequence identity thereto; and a VL region comprising the amino acid sequence of SEQ ID NO: 521, or an amino acid sequence having at least 90%, 91%, 92%, 93%, 94%, 95%, 96%, 97%, 98%, or 99% sequence identity thereto; (d) a CD133 binding agent comprising a VH region comprising the amino acid sequence of SEQ ID NO: 526, or an amino acid sequence having at least 90%, 91%, 92%, 93%, 94%, 95%, 96%, 97%, 98%, or 99% sequence identity thereto; and a VL region comprising the amino acid sequence of SEQ ID NO: 530, or an amino acid sequence having at least 90%, 91%, 92%, 93%, 94%, 95%, 96%, 97%, 98%, or 99% sequence identity thereto; or (e) a CD133 binding agent comprising a VH region comprising the amino acid sequence of SEQ ID NO: 553, or an amino acid sequence having at least 90%, 91%, 92%, 93%, 94%, 95%, 96%, 97%, 98%, or 99% sequence identity thereto; and a VL region comprising the amino acid sequence of SEQ ID NO: 557, or an amino acid sequence having at least 90%, 91%, 92%, 93%, 94%, 95%, 96%, 97%, 98%, or 99% sequence identity thereto.
29. The lipid particle of any of embodiments 1-28, wherein (a) the first targeting moiety comprises a CD133 binding agent comprising a VH region comprising the amino acid sequence of SEQ ID NO: 517, or an amino acid sequence having at least 90%, 91%, 92%, 93%, 94%, 95%, 96%, 97%, 98%, or 99% sequence identity thereto; and (b) the second targeting moiety comprises a CD133 binding agent comprising a VH region comprising the amino acid sequence of SEQ ID NO: 526, or an amino acid sequence having at least 90%, 91%, 92%, 93%, 94%, 95%, 96%, 97%, 98%, or 99% sequence identity thereto.
30. The lipid particle of any of embodiments 1-28, wherein: (a) the first targeting moiety comprises a CD133 binding agent comprising a VH region comprising the amino acid sequence of SEQ ID NO: 526, or an amino acid sequence having at least 90%, 91%, 92%, 93%, 94%, 95%, 96%, 97%, 98%, or 99% sequence identity thereto; and (b) the second targeting moiety comprises a CD133 binding agent comprising a VH region comprising the amino acid sequence of SEQ ID NO: 517, or an amino acid sequence having at least 90%, 91%, 92%, 93%, 94%, 95%, 96%, 97%, 98%, or 99% sequence identity thereto.
31. The lipid particle of any of embodiments 1-30, wherein the first and second targeting moiety bind to distinct epitopes on CD133.
32. A lipid particle, comprising: (a) a first retargeted attachment protein comprising (i) a first paramyxovirus envelope attachment protein; and (ii) a first targeting moiety directed to CD117; and a second targeting moiety directed to CD117; and (b) at least one paramyxovirus fusion (F) protein; and the protein in (a), (b) are exposed on the outside of the lipid bilayer.
33. The lipid particle of embodiments 1-23 and 32, wherein (a) the first targeting moiety comprises a CD117 binding agent comprising a VHH single domain antibody comprising a CDR-H1, a CDR-H2, and a CDR-H3 contained within an amino acid sequence selected from the group consisting of SEQ ID NOs: 512-515; and/or wherein (b) the second targeting moiety comprises a CD117 binding agent comprising a VHH single domain antibody comprising a CDR-H1, a CDR-H2, and a CDR-H3 contained within an amino acid sequence selected from the group consisting of SEQ ID NOs: 512-515.
34. The lipid particle of any of embodiments 1-23, and 32-33, wherein
(a) the first targeting moiety comprising a VHH single domain antibody comprising an amino acid sequence selected from the group consisting of SEQ ID NOs: 512-515, or an amino acid sequence having at least 90%, 91%, 92%, 93%, 94%, 95%, 96%, 97%, 98%, or 99% sequence identity thereto; and/or (b)) the second targeting moiety comprising a VHH single domain antibody comprising an amino acid sequence selected from the group consisting of SEQ ID NOs: 512-515, or an amino acid sequence having at least 90%, 91%, 92%, 93%, 94%, 95%, 96%, 97%, 98%, or 99% sequence identity thereto.
35. The lipid particle of embodiment 33 or embodiment 34, wherein (a) the first targeting moiety comprises a VHH comprising the amino acid sequence set forth in any one of SEQ ID NOS: 512-515; and/or (b) the second targeting moiety comprises a VHH comprising the amino acid sequence set forth in any one of SEQ ID NOS: 512-515.
36. The lipid particle of any of embodiments 1-23, and 32-35, wherein the first and second targeting moiety bind to distinct epitopes on CD117.
37. A lipid particle, comprising: (a) a first retargeted attachment protein comprising (i) a first paramyxovirus envelope attachment protein; and (ii) a first targeting moiety directed to CD8; and a second targeting moiety directed to CD8; and (b) at least one paramyxovirus fusion (F) protein; and the protein in (a), and (b) are exposed on the outside of the lipid bilayer.
37b. A lipid particle comprising: (a) a first retargeted attachment protein comprising (i) an amino acid sequence selected from SEQ ID NO: 584 or 585, or an amino acid sequence having at least 90%, 91%, 92%, 93%, 94%, 95%, 96%, 97%, 98%, or 99% sequence identity thereto, and (b) at least one paramyxovirus fusion (F) protein; and the protein in (a), (b) are exposed on the outside of the lipid bilayer.
37c. A lipid particle comprising: (a) a first retargeted attachment protein comprising (i) an amino acid sequence selected from SEQ ID NO: 584 or 585, or an amino acid sequence having at least 90%, 91%, 92%, 93%, 94%, 95%, 96%, 97%, 98%, or 99% sequence identity thereto; (b) at least one paramyxovirus fusion (F) protein; and the protein in (a), (b) are exposed on the outside of the lipid bilayer; and (c) a second paramyxovirus envelope attachment protein that is a variant paramyxovirus envelope attachment protein comprising one or more amino acid substitutions to reduce the native tropism relative to the wild-type paramyxovirus envelope attachment protein not comprising the one or more amino acid substitutions, optionally wherein the second paramyxovirus envelope attachment protein that is a variant paramyxovirus envelope attachment protein comprises one or more amino acid substitutions corresponding to amino acid substitutions selected from the group consisting of E501A, W504A, Q530A and E533A with reference to numbering set forth in SEQ ID NO:1.
38. The lipid particle of embodiment 37 or 37b or 37c, wherein (i) the first targeting moiety is an scFv and comprises an amino acid sequence selected from the group consisting of SEQ ID NOs: 489, 496, 501, or 508 or an amino acid sequence having at least 90%, 91%, 92%, 93%, 94%, 95%, 96%, 97%, 98%, or 99% sequence identity thereto; and/or (ii) the second targeting moiety is an scFv and comprises an amino acid sequence selected from the group consisting of SEQ ID NOs: 489, 496, 501, or 508, or an amino acid sequence having at least 90%, 91%, 92%, 93%, 94%, 95%, 96%, 97%, 98%, or 99% sequence identity thereto
39. The lipid particle of embodiments 1-23 and 37 or 37c, wherein (a) the first targeting moiety comprises a CD8 binding agent comprising a VHH single domain antibody comprising a CDR-H1, a CDR-H2, and a CDR-H3 contained within an amino acid sequence of SEQ ID NO: 377; and/or wherein (b) the second targeting moiety comprises a CD8 binding agent comprising a VHH single domain antibody comprising a CDR-H1, a CDR-H2, and a CDR-H3 contained within an amino acid sequence of SEQ ID NO: 377.
40. The lipid particle of embodiment 37 or 37b or 37c or 39, wherein (a) the first targeting moiety comprises a VHH comprising the amino acid sequence set forth in SEQ ID NOS: 377; and/or (b) the second targeting moiety comprises a VHH comprising the amino acid sequence set forth in SEQ ID NO: 377.
41. The lipid particle of any of embodiments 1-23, and 37-40, wherein the first and second targeting moiety bind to distinct epitopes on CD8.
42. The lipid particle of any one of embodiments 1-30, further comprising a second paramyxovirus envelope attachment protein that is a variant paramyxovirus envelope attachment protein comprising one or more mutations that reduces the native tropism relative to the wild-type paramyxovirus envelope attachment protein not comprising the one or more mutations.
43. The lipid particle of any of embodiments 16-42, wherein the distinct epitopes are non-overlapping.
44. The lipid particle of any of embodiments 16-43, wherein the first and second targeting moiety bind to the distinct epitopes in a non-competitive manner.
45. The lipid particle of embodiment 1-44, wherein each of the first targeting moiety and the second targeting moiety are independently selected from the group consisting of an antibody or antigen-binding fragment, a DARPin, an Aptamer, an Affimer, an Affibody, a Knottin, an Avimer, a Monobody, an Anticalin, a Fynomer, and a targeting peptide.
46. The lipid particle of any of embodiments 1-45, wherein the first targeting moiety and the second targeting moiety are independently selected from the group consisting of a single domain antibody or a single chain variable fragment (scFv).
47. A lipid particle, comprising: (a) a retargeted attachment protein comprising (i) a first paramyxovirus envelope attachment protein; and (ii) a first and second targeting moiety directed to CD133, (b) a second paramyxovirus envelope attachment protein that is a variant paramyxovirus envelope attachment protein comprising one or more mutations that reduces the native tropism relative to the wild-type paramyxovirus envelope attachment protein not comprising the one or more mutations; and (c) at least one paramyxovirus fusion (F) protein; and wherein the protein in (a), (b) and (c) are exposed on the outside of the lipid bilayer.
48. A lipid particle, comprising: (a) a retargeted attachment protein comprising (i) a first paramyxovirus envelope attachment protein; and (ii) a first and second targeting moiety directed to CD117, (b) a second paramyxovirus envelope attachment protein that is a variant paramyxovirus envelope attachment protein comprising one or more mutations that reduces the native tropism relative to the wild-type paramyxovirus envelope attachment protein not comprising the one or more mutations; and (c) at least one paramyxovirus fusion (F) protein; and wherein the protein in (a), (b) and (c) are exposed on the outside of the lipid bilayer.
49. A lipid particle, comprising: (a) a retargeted attachment protein comprising (i) a first paramyxovirus envelope attachment protein; and (ii) a first and second targeting moiety directed to CD8, (b) a second paramyxovirus envelope attachment protein that is a variant paramyxovirus envelope attachment protein comprising one or more mutations that reduces the native tropism relative to the wild-type paramyxovirus envelope attachment protein not comprising the one or more mutations; and (c) at least one paramyxovirus fusion (F) protein; and wherein the protein in (a), (b) and (c) are exposed on the outside of the lipid bilayer.
50. The lipid particle of embodiments 24-49, wherein the first paramyxovirus envelope attachment protein is a variant paramyxovirus envelope attachment protein.
51. The lipid particle of embodiment 50, wherein the variant paramyxovirus envelope attachment protein comprises one or more mutations that reduces native tropism relative to the wild-type paramyxovirus envelope attachment protein not comprising the one or more mutations.
52. The lipid particle of any of embodiments 24-51, wherein the second paramyxovirus envelope attachment protein is a variant paramyxovirus envelope attachment protein.
53. The lipid particle of embodiment 52, wherein the variant paramyxovirus envelope attachment protein comprises one or more mutations that reduces native tropism relative to the wild-type paramyxovirus envelope attachment protein not comprising the one or more mutations.
54. The lipid particle of any of embodiments 1-53, wherein the first targeting moiety is selected from the group consisting of a single domain antibody or a single chain variable fragment (scFv).
55. The lipid particle of any of embodiments 1-54, wherein the second targeting moiety is selected from the group consisting of a single domain antibody or a single chain variable fragment (scFv).
56. The lipid particle of embodiments 46 and embodiment 54, or 55, wherein the single domain antibody is a VHH.
57. The lipid particle of any of embodiments 1-56, wherein the first variant paramyxovirus envelope attachment protein and the second variant paramyxovirus envelope attachment protein are the same.
58. The lipid particle of any of embodiments 1-57, wherein the first paramyxovirus envelope attachment protein and the second paramyxovirus envelope attachment protein are different.
59. The lipid particle of any of embodiments 1-58, wherein the first paramyxovirus envelope attachment protein is an envelope attachment protein from a Nipah virus, Hendra virus, or Measles virus, or is a variant or biologically active portion thereof of any of the foregoing.
60. The lipid particle of any of embodiments 1-59, wherein the first paramyxovirus envelope attachment protein is a wild-type paramyxovirus G protein, H protein or HN protein or is a variant or biologically active portion of any of the foregoing.
61. The lipid particle of embodiment 59 or embodiment 60, wherein the first paramyxovirus envelope attachment protein is a wild-type Nipah virus G (NiV-G) protein or is a variant or biologically active portion of a NiV-G.
62. The lipid particle of any of embodiments 59-61, wherein the first paramyxovirus envelope attachment protein is a variant NiV-G that is a variant or a biologically active portion of a wild-type NiV-G.
63. The lipid particle of any of embodiments 1-62, wherein the second paramyxovirus envelope attachment protein is an envelope attachment protein from a Nipah virus, Hendra virus, or Measles virus, or is a variant or biologically active portion of any of the foregoing.
64. The lipid particle of any of embodiments 1-63, wherein the second paramyxovirus envelope attachment protein is a wild-type paramyxovirus G protein, H protein or HN protein or is a variant or biologically active portion of any of the foregoing.
65. The lipid particle of embodiment 63 or embodiment 64, wherein the second paramyxovirus envelope attachment protein is a wild-type Nipah virus G (NiV-G) protein or is a variant or a biologically active portion of a NiV-G.
66. The lipid particle of any of embodiments 1-65, wherein the second paramyxovirus envelope attachment protein is a variant NiV-G that is a variant or a biologically active portion of a wild-type NiV-G.
67. The lipid particle of any of embodiments 3-62, wherein the second paramyxovirus envelope attachment protein is a variant paramyxovirus envelope glycoprotein from a Nipah virus, Hendra virus, or Measles virus or a biologically active portion thereof.
68. The lipid particle of any of embodiments 3-62 and 67, wherein the second paramyxovirus envelope attachment protein is a variant of a wild-type paramyxovirus G protein, H protein or HN protein or a biologically active portion thereof.
69. The lipid particle of any one of embodiments 65, and 66, wherein the variant is a variant NiV-G that is a variant of a wild-type Nipah virus G (NiV-G) protein or a biologically active portion thereof.
70. The lipid particle of embodiment 62, embodiment 66, or embodiment 69, wherein the variant NiV-G is truncated by up to 40 contiguous amino acids at or near the N-terminus of the wild-type NiV-G set forth in SEQ ID NO:1.
71. The lipid particle of any of embodiments 62, 66, 69 and 70, wherein the variant NiV-G has a truncation of amino acids 2-34 of the wild-type NiV-G set forth in SEQ ID NO:1.
72. The lipid particle of any of embodiments 62, 65 and 69-71, wherein the variant NiV-G exhibits reduced binding to Ephrin B2 or Ephrin B3.
73. The lipid particle of embodiment 72, wherein the variant NiV-G comprises: one or more amino acid substitutions corresponding to amino acid substitutions selected from the group consisting of E501A, W504A, Q530A and E533A with reference to numbering set forth in SEQ ID NO:1.
74. The lipid particle of embodiment 72 or embodiment 73, wherein the variant NiV-G comprises amino acid substitutions E501A, W504A, Q530A and E533A with reference to numbering set forth in SEQ ID NO:1.
75. The lipid particle of any of embodiments 62, 66 and 69-74, wherein the variant NiV-G has the amino acid sequence set forth in SEQ ID NO: 228 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:228.
76. The lipid particle of any of embodiments 62, 66 and 69-74, wherein the variant NiV-G has the amino acid sequence set forth in SEQ ID NO:228.
77. The lipid particle of any of embodiments 1-76, wherein the at least one paramyxovirus fusion (F) protein is an F protein from a henipavirus or is a biologically active portion thereof or variant thereof.
78. The lipid particle of embodiment 77, wherein the henipavirus is a Hendra virus. 79. The lipid particle of embodiment 77, wherein the henipavirus is a Nipah virus.
80. The lipid particle of any of embodiments 1-79, wherein the paramyxovirus F protein is a wild-type NiV-F protein or a variant or a biologically active portion thereof.
81. The lipid particle of any of embodiments 1-80, wherein the paramyxovirus F protein is a variant NiV-F that is a variant or a biologically active portion of a wild-type NiV-F protein.
82. The lipid particle of embodiment 81, wherein the variant NiV-F is truncated by up to 22 contiguous amino acids the at the C-terminus of the wild-type NiV-F set forth in SEQ ID NO:235, optionally not include the initial methionine.
83. The lipid particle of embodiment 81 or embodiment 82, wherein the variant NiV-F protein is a truncated NiV-F that lacks amino acids 525-546 of SEQ ID NO:235.
84. The lipid particle of any of embodiments 81-83, wherein the variant NiV-F has the amino acid sequence set forth in SEQ ID NO: 227 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:227.
85. The lipid particle of any of embodiments 81-84, wherein the variant NiV-F has the amino acid sequence set forth in SEQ ID NO:227.
86. The lipid particle of any of embodiments 1-85, wherein the paramyxovirus F protein is an F0 precursor or is a proteolytically cleaved form thereof comprising F1 and F2 subunits.
87. The lipid particle of embodiment 86, wherein the proteolytically cleaved form is a cathepsin L cleavage product.
88. The lipid particle of any of embodiments 1-87, wherein the paramyxovirus envelope attachment protein and the first targeting moiety and second targeting moiety are linked via one or more linkers.
89. The lipid particle of embodiment 88, wherein the one or more linkers are one or more peptide linker.
90. The lipid particles of any of embodiments 1-89, wherein the retargeted attachment protein comprises in order: Paramyxovirus attachment protein-first linker-first targeting moiety-second linker-second targeting moiety.
91. The lipid particle of embodiment 90, wherein the first linker and second linker is independently a peptide linker.
92. The lipid particle of embodiment 91, wherein the first linker and second linker are the same.
93. The lipid particle of embodiment 91, wherein the first linker and second linker are different.
94. The lipid particle of embodiment 89 or embodiment 91, wherein the peptide linker is 2 to 65 amino acids in length.
95. The lipid particle of any of embodiments 91-94, wherein the peptide linker is a flexible linker that comprises GS, GGS, GGGGS, GGGGGS or combinations thereof.
96. The lipid particle of any of embodiments 91-95, wherein the peptide linker is selected from: (GGS)n, wherein n is 1 to 10; (GGGGS)n, wherein n is 1 to 10; or (GGGGGS)n, wherein n is 1 to 6.
97. The lipid particle of any of embodiments 1-94, wherein the peptide linker is selected from SEQ ID NOs: 589-592.
98. The lipid particle of any of embodiments 1-97, wherein the lipid particle further comprises one or more additional paramyxovirus envelope attachment glycoproteins embedded in the lipid bilayer.
99. The lipid particle of embodiment 98, wherein the one or more additional paramyxovirus envelope attachment glycoproteins is a retargeted attachment protein comprising a paramyxovirus envelope attachment protein and a further targeting moiety.
100. The lipid particle of any of embodiments 1-99, wherein the at least one paramyxovirus fusion (F) protein exhibits fusogenic activity with a target cell upon binding of at least one paramyxovirus envelope attachment protein to the target molecule on the target cell.
101. The lipid particle of any of embodiments 1-100, wherein the lipid particle comprises a viral nucleic acid.
102. The lipid particle of embodiment 101, wherein the viral nucleic acid comprises one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3).
103. The lipid particle of any of embodiments 1-102, wherein the lipid particle is a viral vector.
104. The lipid particle of any of embodiments 1-103, that is a retroviral vector.
105. The lipid particle of any of embodiments 1-103, that is a lentiviral vector.
106. The lipid particle of any of embodiments 1-102, wherein the lipid particle is devoid of viral genomic DNA.
107. The lipid particle of any of embodiments 1-102 and 106, that is a viral-like particle.
108. The lipid particle of any of embodiments 1-102, 106 and 107 that is a retroviral-like particle.
109. The lipid particle of any of embodiments 1-102, 106 and 107 that is a lentiviral-like particle.
110. The lipid particle of any of embodiments 1-109, wherein the lipid particle is produced as a preparation with increased titer compared to a reference lipid particle preparation that is similarly produced but with only the first retargeted attachment protein.
111. The lipid particle of embodiment 110, wherein the titer is increased by at or greater than 1.2-fold, 1.3-fold, 1.4-fold, 1.5-fold, 1.6-fold, 1.7-fold, 1.8-fold, 2-fold, 2.5-fold, 3-fold, 4-fold, 5-fold, 6-fold, 7-fold, 8-fold, 9-fold, 10-fold, or more.
112. The lipid particle of any of embodiments 1-111, further comprising an exogenous agent for delivery to a target cell.
113. The lipid particle of embodiment 112, wherein the exogenous agent is present in the lumen.
114. The lipid particle of embodiment 112 or embodiment 113, wherein the exogenous agent is a protein or a nucleic acid, optionally wherein the nucleic acid is a DNA or RNA.
115. The lipid particle of any of embodiments 112-114, wherein the exogenous agent is a nucleic acid encoding a cargo for delivery to the target cell.
116. The lipid particle of any of embodiments 112-115, wherein the exogenous agent is or encodes a therapeutic agent, a diagnostic agent, or a genome-modifying enzyme.
117. The lipid particle of any of embodiments 112-116, wherein the exogenous agent encodes a membrane protein, optionally wherein the membrane protein is an antigen receptor for targeting cells expressed by or associated with a disease or condition.
118. The lipid particle of embodiment 117, wherein the membrane protein is a chimeric antigen receptor (CAR).
119. The lipid particle of any of embodiments 112-116, wherein the exogenous agent is a nucleic acid comprising a payload gene for correcting a genetic deficiency, optionally a genetic deficiency in the target cell, optionally wherein the genetic deficiency is associated with a liver cell or a hepatocyte.
120. The lipid particle of any of embodiments 112-119, wherein binding of the paramyxovirus envelope attachment protein or biologically active portion thereof to a target molecule expressed on the surface of a target cell mediates fusion of the particle with the target cell and delivery of the exogenous agent to the target cell.
121. The lipid particle of any of embodiments 112-120, wherein at or greater than 10%, 20%, 30%, 40%, 50%, 60% of the target cells are delivered the exogenous agent.
122. The lipid particle of any of embodiments 112-121, wherein delivery of the exogenous cell to the target cell is increased compared to a reference particle preparation that is similarly produced but with only a first retargeted attachment protein.
123. The lipid particle of embodiment 122, wherein the delivery to the target cell is increased by at or greater than 1.2-fold, 1.3-fold, 1.4-fold, 1.5-fold, 1.6-fold, 1.7-fold, 1.8-fold, 2-fold, 2.5-fold, 3-fold, 4-fold, 5-fold, 6-fold, 7-fold, 8-fold, 9-fold, 10-fold, or more.
124. A producer cell comprising (a) a nucleic acid encoding a retargeted attachment protein comprising a paramyxovirus envelope attachment protein linked to (i) a first targeting moiety directed to a first target molecule expressed on the surface of a target cell, and (ii) a second targeting moiety directed to a second target molecule expressed on the surface of a target cell; and (b) a nucleic acid encoding at least one paramyxovirus fusion (F) protein.
125. The producer cell of embodiment 124, wherein the cell further comprises a viral nucleic acid(s).
126. The producer cell of any of embodiment 125, wherein the viral nucleic acid(s) are lentiviral nucleic acids.
127. The producer cell of any of embodiments 124-126, wherein the cell is a mammalian cell.
128. The producer cell of any of embodiments 124-127, wherein the producer cell is selected from the group consisting of CHO cells, BHK cells, MDCK cells, C3H 10T½ cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.
129. The producer cell of any of embodiments 124-128, wherein the producer cell comprises 293T cells.
130. The producer cell of any of embodiments 125-129, wherein the viral nucleic acid(s) lacks one or more genes involved in viral replication.
131. The producer cell of any of embodiments 125-130, wherein the viral nucleic acid comprises a nucleic acid encoding a viral packaging protein selected from one or more of Gag, Pol, Rev and Tat.
132. The producer cell of any of embodiments 125-131, wherein the viral nucleic acid comprises: one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3).
133. A method of making a lipid particle, comprising: a) providing a producer cell of any of embodiments 124-132; b) culturing the cell under conditions that allow for production of the lipid particle, and c) separating, enriching, or purifying the lipid particle from the cell, thereby making the lipid particle.
134. The method of embodiment 133, wherein the lipid particle is a pseudotyped lentiviral vector.
135. A lipid particle produced by the method of embodiment 133 or embodiment 134.
136. A composition comprising a plurality of lipid particles of any of embodiments 1-123 and 135.
137. The composition of embodiment 136, further comprising a pharmaceutically acceptable carrier.
138. A method of transducing a cell comprising contacting a cell with a lipid particle of any of embodiments 1-123 and 135 or a composition of embodiment 136 or embodiment 137.
139. A method of delivering an exogenous agent into a target cell, the method comprising contacting a lipid particle of any of embodiments 112-123 and 135 or a composition of embodiment 136 or embodiment 137 with a target cell.
140. The method of embodiment 138 or embodiment 139, wherein the contacting is in vitro or ex vivo.
141. The method of embodiment 138 or embodiment 139, wherein the contacting is in vivo in a subject.
142. A method of delivering an exogenous agent to a cell in a subject, the method comprising administering to the subject a lipid particle of any of embodiments 112-123 and 135 or a composition of embodiment 136 or embodiment 137.
143. The method of embodiment 142, wherein the exogenous agent is or encodes a therapeutic agent for treating a disease or condition in the subject.
144. A method of treatment, the method comprising administering to a subject a lipid particle of any of embodiments 112-123 and 135 or a composition of embodiment 136 or embodiment 137.
145. The method of any of embodiments 139-144, wherein the exogenous agent is or encodes a membrane protein, optionally a chimeric antigen receptor, for targeting an antigen associated with a disease or condition in the subject.
146. The method of any of embodiments 139-144, wherein the exogenous agent is for use in gene therapy to correct a genetic deficiency or replaces a deficient or missing gene in the subject.
147. The method of any of embodiments 142-146, wherein the subject is a human subject.
148. The method of any of embodiments 138-147, wherein the method further comprises administering to the subject one or more agents that stimulate mobilization of bone marrow cells from the bone marrow to the peripheral blood.
149. The method of any of claims 138-148 embodiments wherein the subject has previously been administered one or more agents that stimulate mobilization of bone marrow cells from the bone marrow to the peripheral blood.
150. The method of embodiment 148 or 149, wherein the one or more agents that stimulate mobilization are selected from the group consisting of stem cell factor (SCF), small molecule VLA-4 inhibitor BI05192, BOP (N-(benzenesulfonyl)-L-prolyl-L-0-(1-pyrrolidinylcarbonyl) tyrosine), heparin, granulocyte colony-stimulating factor (G-CSF), MGTA-145, and plerixafor (AMD3100).
151. The method of any of embodiments 148-150, wherein the one or more agents that stimulate mobilization comprise G-CSF.
152. The method of any of embodiments 148-150, wherein the one or more agents that stimulate mobilization comprise plerixafor.

### Exemplary Embodiments B

1. A lipid particle, comprising: (a) a retargeted attachment protein comprising a paramyxovirus envelope attachment protein linked to (i) a first targeting moiety directed to a first target molecule expressed on the surface of a target cell, and (ii) a second targeting moiety directed to a second target molecule expressed on the surface of a target cell; and (b) at least one paramyxovirus fusion (F) protein; and wherein the protein in (a), and (b) are exposed on the outside of the lipid bilayer.
2. The lipid particle of embodiment 1, wherein the paramyxovirus attachment protein is a variant paramyxovirus envelope attachment protein comprising one or more mutations to reduce native tropism relative to the wild-type paramyxovirus envelope attachment protein not comprising the one or more mutations.
3. The lipid particle of embodiment 1 or 2, wherein the paramyxovirus envelope attachment protein is a first paramyxovirus envelope attachment protein and the lipid particle further comprises a second paramyxovirus envelope attachment protein, wherein the second paramyxovirus envelope attachment protein is a variant paramyxovirus envelope attachment protein comprising one or more mutations to reduce native tropism relative to the wild-type paramyxovirus envelope attachment protein not comprising the one or more mutations, wherein the second paramyxovirus envelope attachment protein is exposed on the outside of the lipid bilayer.
4. The lipid particle of any one of embodiments 1-3, wherein the targeting one or both of the first target molecule and the second target molecule does not activate or inhibit, induce a phenotype change (for example maturation and/or differentiation), induce proliferation, and/or induce apoptosis of said target cell.
5. A lipid particle, comprising: (a) a retargeted attachment protein comprising a first paramyxovirus envelope attachment protein operably linked to (i) a first targeting moiety directed to a first target molecule expressed on the surface of a target cell, and (ii) a second targeting moiety directed to a second target molecule expressed on the surface of a target cell, (b) a second paramyxovirus envelope attachment protein that is a variant paramyxovirus envelope attachment protein comprising one or more amino acid substitutions to reduce the native tropism relative to the wild-type paramyxovirus envelope attachment protein not comprising the one or more amino acid substitutions; and (c) at least one paramyxovirus fusion (F) protein; and wherein the protein in (a), (b) and (c) are exposed on the outside of the lipid bilayer.
6. The lipid particle of any of embodiments 1-5, wherein the first paramyxovirus envelope attachment protein is a variant paramyxovirus envelope attachment protein.
7. The lipid particle of embodiment 6, wherein the variant paramyxovirus envelope attachment protein comprises one or more amino acid substitutions that reduces native tropism relative to the wild-type paramyxovirus envelope attachment protein not comprising the one or more amino acid substitutions.
8. The lipid particle of any of embodiments 1-7, wherein the first and second paramyxovirus envelope attachment protein are the same.
9. The lipid particle of any of embodiments 1-8, wherein the second paramyxovirus envelope attachment protein is not linked or fused to a non-viral heterologous moiety that is a cell-specific targeting domain or functional domain, optionally wherein the second paramyxovirus envelope attachment protein is not linked or fused to a cell-specific targeting domain that is a targeting moiety directed to a target molecule expressed on the surface of a target cell.
10. The lipid particle of embodiments 1-9, wherein the first and second targeting moiety are each not selected from the group consisting of a cytokine, growth factor, hormone, neurotransmitter, apoptosis ligand and their combinations.
11. The lipid particle of any of embodiments 1-10, wherein targeting one or both of the first target molecule and the second target molecule does not modulate or induce a signal in the target cell.
12. The lipid particle of any of embodiments 1-11, wherein the first and second targeting moieties each bind to a cell surface molecule present on a target cell.
13. The lipid particle of any of embodiments 1-12, wherein the first targeting moiety binds to a cell surface molecule present on a first target cell, and the second targeting moiety binds a surface molecule present on a second target cell.
14. The lipid particle of any of embodiments 1-13, wherein the first and second target molecule are different target molecules.
15. The lipid particle of any of embodiments 1-13, wherein the first and second target molecule are the same target molecule.
16. The lipid particle of embodiment 15, wherein the first and second targeting moiety bind distinct epitopes of the same target molecule.
17. The lipid particle of any of embodiments 1-16, wherein the cell surface molecule is a protein, glycan, or lipid.
18. The lipid particle of any of embodiments 1-17, wherein the target cell is selected from the group consisting of tumor-infiltrating lymphocytes, T cells, neoplastic or tumor cells, virus-infected cells, stem cells, central nervous system (CNS) cells, hematopoietic stem cells (HSCs), and liver cells.
19. The lipid particle of any of embodiments 1-18, wherein the target cell is selected from the group consisting of a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+B cell, a CD19+B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.
20. The lipid particle of any of embodiments 1-19, wherein the target cell is a hepatocyte.
21. The lipid particle of embodiment 20, wherein the cell surface molecule is selected from the group consisting of CD34, CD117, and CD133.
22. The lipid particle of any of embodiments 1-19, wherein the target cell is a T cell.
23. The lipid particle of embodiment 22, wherein the cell surface molecule is selected from the group consisting of CD3, CD4, CD7 CD8, ASCT2, CD105, CD110, CD117, CD133, CD146, CD164, CD34, CD46, CD49f, CD90, EPCR, and ITGA3.
24. The lipid particle of any of embodiments 5-23, wherein the second paramyxovirus envelope attachment protein that is a variant paramyxovirus envelope attachment protein comprises one or more amino acid substitutions corresponding to amino acid substitutions selected from the group consisting of E501A, W504A, Q530A and E533A with reference to numbering set forth in SEQ ID NO: 1.
25. A lipid particle, comprising: (a) a first retargeted attachment protein comprising (i) a first paramyxovirus envelope attachment protein; and (ii) a first targeting moiety directed to CD133; and a second targeting moiety directed to CD133; and (b) at least one paramyxovirus fusion (F) protein; and the protein in (a), and (b) are exposed on the outside of the lipid bilayer.
26. The lipid particle of any of embodiments 1-25, wherein: (i) the first targeting moiety is an scFv and comprises an amino acid sequence selected from the group consisting of SEQ ID NOs: 516, 525, 534, 543, and 552, or an amino acid sequence having at least 90%, 91%, 92%, 93%, 94%, 95%, 96%, 97%, 98%, or 99% sequence identity thereto; and/or (ii) the second targeting moiety is an scFv and comprises an amino acid sequence selected from the group consisting of SEQ ID NOs: 516, 525, 534, 543, and 552, or an amino acid sequence having at least 90%, 91%, 92%, 93%, 94%, 95%, 96%, 97%, 98%, or 99% sequence identity thereto.
27. The lipid particle of any of embodiments 1-25, wherein: (i) the first targeting moiety comprises: (a) a CD133 binding agent comprising a CDR-H1, a CDR-H2, and a CDR-H3 comprising the amino acid sequences of SEQ ID NOs: 536, 537, and 538, respectively, and a CDR-L1, a CDR-L2, and a CDR-L3 comprising the amino acid sequences of SEQ ID NOs: 540, 541, and 542, respectively; (b) a CD133 binding agent comprising a CDR-H1, a CDR-H2, and a CDR-H3 comprising the amino acid sequences of SEQ ID NOs: 545, 546, and 547, respectively, and a CDR-L1, a CDR-L2, and a CDR-L3 comprising the amino acid sequences of SEQ ID NOs: 549, 550, and 551, respectively; (c) a CD133 binding agent comprising a CDR-H1, a CDR-H2, and a CDR-H3 comprising the amino acid sequences of SEQ ID NOs: 518, 519, and 520, respectively, and a CDR-L1, a CDR-L2, and a CDR-L3 comprising the amino acid sequences of SEQ ID NOs: 522, 523, and 524, respectively; (d) a CD133 binding agent comprising a CDR-H1, a CDR-H2, and a CDR-H3 comprising the amino acid sequences of SEQ ID NOs: 527, 528, and 529, respectively, and a CDR-L1, a CDR-L2, and a CDR-L3 comprising the amino acid sequences of SEQ ID NOs: 531, 532, and 533, respectively; or (e) a CD133 binding agent comprising a CDR-H1, a CDR-H2, and a CDR-H3 comprising the amino acid sequences of SEQ ID NOs: 554, 555, and 556, respectively, and a CDR-L1, a CDR-L2, and a CDR-L3 comprising the amino acid sequences of SEQ ID NOs: 558, 559, and 560, respectively; and/or (ii) the second targeting moiety comprises: (a) a CD133 binding agent comprising a CDR-H1, a CDR-H2, and a CDR-H3 comprising the amino acid sequences of SEQ ID NOs: 536, 537, and 538, respectively, and a CDR-L1, a CDR-L2, and a CDR-L3 comprising the amino acid sequences of SEQ ID NOs: 540, 541, and 542, respectively; (b) a CD133 binding agent comprising a CDR-H1, a CDR-H2, and a CDR-H3 comprising the amino acid sequences of SEQ ID NOs: 545, 546, and 547, respectively, and a CDR-L1, a CDR-L2, and a CDR-L3 comprising the amino acid sequences of SEQ ID NOs: 549, 550, and 551, respectively; (c) a CD133 binding agent comprising a CDR-H1, a CDR-H2, and a CDR-H3 comprising the amino acid sequences of SEQ ID NOs: 518, 519, and 520, respectively, and a CDR-L1, a CDR-L2, and a CDR-L3 comprising the amino acid sequences of SEQ ID NOs: 522, 523, and 524, respectively; (d) a CD133 binding agent comprising a CDR-H1, a CDR-H2, and a CDR-H3 comprising the amino acid sequences of SEQ ID NOs: 527, 528, and 529, respectively, and a CDR-L1, a CDR-L2, and a CDR-L3 comprising the amino acid sequences of SEQ ID NOs: 531, 532, and 533, respectively; or (e) a CD133 binding agent comprising a CDR-H1, a CDR-H2, and a CDR-H3 comprising the amino acid sequences of SEQ ID NOs: 554, 555, and 556, respectively, and a CDR-L1, a CDR-L2, and a CDR-L3 comprising the amino acid sequences of SEQ ID NOs: 558, 559, and 560, respectively.
28. The lipid particle of any of embodiments 1-25, wherein: (i) the first targeting moiety comprises: (a) a CD133 binding agent comprising a CDR-H1, a CDR-H2, and a CDR-H3 comprising the amino acid sequences of SEQ ID NOs: 289, 565, and 538, respectively, and a CDR-L1, a CDR-L2, and a CDR-L3 comprising the amino acid sequences of SEQ ID NOs: 540, 541, and 542, respectively; (b) a CD133 binding agent comprising a CDR-H1, a CDR-H2, and a CDR-H3 comprising the amino acid sequences of SEQ ID NOs: 566, 567, and 547, respectively, and a CDR-L1, a CDR-L2, and a CDR-L3 comprising the amino acid sequences of SEQ ID NOs: 549, 550, and 551, respectively; (c) a CD133 binding agent comprising a CDR-H1, a CDR-H2, and a CDR-H3 comprising the amino acid sequences of SEQ ID NOs: 568, 569, and 520, respectively, and a CDR-L1, a CDR-L2, and a CDR-L3 comprising the amino acid sequences of SEQ ID NOs: 522, 523, and 524, respectively; (d) a CD133 binding agent comprising a CDR-H1, a CDR-H2, and a CDR-H3 comprising the amino acid sequences of SEQ ID NOs: 570, 571, and 529, respectively, and a CDR-L1, a CDR-L2, and a CDR-L3 comprising the amino acid sequences of SEQ ID NOs: 531, 532, and 533, respectively; or (e) a CD133 binding agent comprising a CDR-H1, a CDR-H2, and a CDR-H3 comprising the amino acid sequences of SEQ ID NOs: 572, 573, and 556, respectively, and a CDR-L1, a CDR-L2, and a CDR-L3 comprising the amino acid sequences of SEQ ID NOs: 558, 559, and 560, respectively; and/or (ii) the second targeting moiety comprises: (a) a CD133 binding agent comprising a CDR-H1, a CDR-H2, and a CDR-H3 comprising the amino acid sequences of SEQ ID NOs: 289, 565, and 538, respectively, and a CDR-L1, a CDR-L2, and a CDR-L3 comprising the amino acid sequences of SEQ ID NOs: 540, 541, and 542, respectively; (b) a CD133 binding agent comprising a CDR-H1, a CDR-H2, and a CDR-H3 comprising the amino acid sequences of SEQ ID NOs: 566, 567, and 547, respectively, and a CDR-L1, a CDR-L2, and a CDR-L3 comprising the amino acid sequences of SEQ ID NOs: 549, 550, and 551, respectively; (c) a CD133 binding agent comprising a CDR-H1, a CDR-H2, and a CDR-H3 comprising the amino acid sequences of SEQ ID NOs: 568, 569, and 520, respectively, and a CDR-L1, a CDR-L2, and a CDR-L3 comprising the amino acid sequences of SEQ ID NOs: 522, 523, and 524, respectively; (d) a CD133 binding agent comprising a CDR-H1, a CDR-H2, and a CDR-H3 comprising the amino acid sequences of SEQ ID NOs: 570, 571, and 529, respectively, and a CDR-L1, a CDR-L2, and a CDR-L3 comprising the amino acid sequences of SEQ ID NOs: 531, 532, and 533, respectively; or (e) a CD133 binding agent comprising a CDR-H1, a CDR-H2, and a CDR-H3 comprising the amino acid sequences of SEQ ID NOs: 572, 573, and 556, respectively, and a CDR-L1, a CDR-L2, and a CDR-L3 comprising the amino acid sequences of SEQ ID NOs: 558, 559, and 560, respectively.
29. The lipid particle of any of embodiments 1-25, wherein: (i) the first targeting moiety comprises: (a) a CD133 binding agent comprising a heavy chain variable (VH) region comprising the amino acid sequence of SEQ ID NO: 535, or an amino acid sequence having at least 90%, 91%, 92%, 93%, 94%, 95%, 96%, 97%, 98%, or 99% sequence identity thereto; and a light chain variable (VL) region comprising the amino acid sequence of SEQ ID NO: 539, or an amino acid sequence having at least 90%, 91%, 92%, 93%, 94%, 95%, 96%, 97%, 98%, or 99% sequence identity thereto; (b) a CD133 binding agent comprising a VH region comprising the amino acid sequence of SEQ ID NO: 544, or an amino acid sequence having at least 90%, 91%, 92%, 93%, 94%, 95%, 96%, 97%, 98%, or 99% sequence identity thereto; and a VL region comprising the amino acid sequence of SEQ ID NO: 548, or an amino acid sequence having at least 90%, 91%, 92%, 93%, 94%, 95%, 96%, 97%, 98%, or 99% sequence identity thereto; (c) a CD133 binding agent comprising a VH region comprising the amino acid sequence of SEQ ID NO: 517, or an amino acid sequence having at least 90%, 91%, 92%, 93%, 94%, 95%, 96%, 97%, 98%, or 99% sequence identity thereto; and a VL region comprising the amino acid sequence of SEQ ID NO: 521, or an amino acid sequence having at least 90%, 91%, 92%, 93%, 94%, 95%, 96%, 97%, 98%, or 99% sequence identity thereto; (d) a CD133 binding agent comprising a VH region comprising the amino acid sequence of SEQ ID NO: 526, or an amino acid sequence having at least 90%, 91%, 92%, 93%, 94%, 95%, 96%, 97%, 98%, or 99% sequence identity thereto; and a VL region comprising the amino acid sequence of SEQ ID NO: 530, or an amino acid sequence having at least 90%, 91%, 92%, 93%, 94%, 95%, 96%, 97%, 98%, or 99% sequence identity thereto; or (e) a CD133 binding agent comprising a VH region comprising the amino acid sequence of SEQ ID NO: 553, or an amino acid sequence having at least 90%, 91%, 92%, 93%, 94%, 95%, 96%, 97%, 98%, or 99% sequence identity thereto; and a VL region comprising the amino acid sequence of SEQ ID NO: 557, or an amino acid sequence having at least 90%, 91%, 92%, 93%, 94%, 95%, 96%, 97%, 98%, or 99% sequence identity thereto; and/or (ii) the second targeting moiety comprises: (a) a CD133 binding agent comprising a heavy chain variable (VH) region comprising the amino acid sequence of SEQ ID NO: 535, or an amino acid sequence having at least 90%, 91%, 92%, 93%, 94%, 95%, 96%, 97%, 98%, or 99% sequence identity thereto; and a light chain variable (VL) region comprising the amino acid sequence of SEQ ID NO: 539, or an amino acid sequence having at least 90%, 91%, 92%, 93%, 94%, 95%, 96%, 97%, 98%, or 99% sequence identity thereto; (b) a CD133 binding agent comprising a VH region comprising the amino acid sequence of SEQ ID NO: 544, or an amino acid sequence having at least 90%, 91%, 92%, 93%, 94%, 95%, 96%, 97%, 98%, or 99% sequence identity thereto; and a VL region comprising the amino acid sequence of SEQ ID NO: 548, or an amino acid sequence having at least 90%, 91%, 92%, 93%, 94%, 95%, 96%, 97%, 98%, or 99% sequence identity thereto; (c) a CD133 binding agent comprising a VH region comprising the amino acid sequence of SEQ ID NO: 517, or an amino acid sequence having at least 90%, 91%, 92%, 93%, 94%, 95%, 96%, 97%, 98%, or 99% sequence identity thereto; and a VL region comprising the amino acid sequence of SEQ ID NO: 521, or an amino acid sequence having at least 90%, 91%, 92%, 93%, 94%, 95%, 96%, 97%, 98%, or 99% sequence identity thereto; (d) a CD133 binding agent comprising a VH region comprising the amino acid sequence of SEQ ID NO: 526, or an amino acid sequence having at least 90%, 91%, 92%, 93%, 94%, 95%, 96%, 97%, 98%, or 99% sequence identity thereto; and a VL region comprising the amino acid sequence of SEQ ID NO: 530, or an amino acid sequence having at least 90%, 91%, 92%, 93%, 94%, 95%, 96%, 97%, 98%, or 99% sequence identity thereto; or (e) a CD133 binding agent comprising a VH region comprising the amino acid sequence of SEQ ID NO: 553, or an amino acid sequence having at least 90%, 91%, 92%, 93%, 94%, 95%, 96%, 97%, 98%, or 99% sequence identity thereto; and a VL region comprising the amino acid sequence of SEQ ID NO: 557, or an amino acid sequence having at least 90%, 91%, 92%, 93%, 94%, 95%, 96%, 97%, 98%, or 99% sequence identity thereto.
30. The lipid particle of any of embodiments 1-29, wherein (a) the first targeting moiety comprises a CD133 binding agent comprising a VH region comprising the amino acid sequence of SEQ ID NO: 517, or an amino acid sequence having at least 90%, 91%, 92%, 93%, 94%, 95%, 96%, 97%, 98%, or 99% sequence identity thereto; and (b) the second targeting moiety comprises a CD133 binding agent comprising a VH region comprising the amino acid sequence of SEQ ID NO: 526, or an amino acid sequence having at least 90%, 91%, 92%, 93%, 94%, 95%, 96%, 97%, 98%, or 99% sequence identity thereto.
31. The lipid particle of any of embodiments 1-29, wherein: (a) the first targeting moiety comprises a CD133 binding agent comprising a VH region comprising the amino acid sequence of SEQ ID NO: 526, or an amino acid sequence having at least 90%, 91%, 92%, 93%, 94%, 95%, 96%, 97%, 98%, or 99% sequence identity thereto; and (b) the second targeting moiety comprises a CD133 binding agent comprising a VH region comprising the amino acid sequence of SEQ ID NO: 517, or an amino acid sequence having at least 90%, 91%, 92%, 93%, 94%, 95%, 96%, 97%, 98%, or 99% sequence identity thereto.
32. The lipid particle of any of embodiments 1-31, wherein the first and second targeting moiety bind to distinct epitopes on CD133.
33. The lipid particle of any of embodiments 25-32, further comprising a second paramyxovirus envelope attachment protein that is a variant paramyxovirus envelope attachment protein comprising one or more amino acid substitutions to reduce the native tropism relative to the wild-type paramyxovirus envelope attachment protein not comprising the one or more amino acid substitutions.
34. The lipid particle of embodiment 33, wherein the second paramyxovirus envelope attachment protein that is a variant paramyxovirus envelope attachment protein comprises one or more amino acid substitutions corresponding to amino acid substitutions selected from the group consisting of E501A, W504A, Q530A and E533A with reference to numbering set forth in SEQ ID NO:1.
35. A lipid particle, comprising: (a) a first retargeted attachment protein comprising (i) a first paramyxovirus envelope attachment protein; and (ii) a first targeting moiety directed to CD117; and a second targeting moiety directed to CD117; and (b) at least one paramyxovirus fusion (F) protein; and the protein in (a), (b) are exposed on the outside of the lipid bilayer.
36. The lipid particle of embodiments 1-25 and 35, wherein (a) the first targeting moiety comprises a CD117 binding agent comprising a VHH single domain antibody comprising a CDR-H1, a CDR-H2, and a CDR-H3 contained within an amino acid sequence selected from the group consisting of SEQ ID NOs: 512-515; and/or wherein (b) the second targeting moiety comprises a CD117 binding agent comprising a VHH single domain antibody comprising a CDR-H1, a CDR-H2, and a CDR-H3 contained within an amino acid sequence selected from the group consisting of SEQ ID NOs: 512-515.
37. The lipid particle of any of embodiments 1-25, and 35-36, wherein (a) the first targeting moiety comprising a VHH single domain antibody comprising an amino acid sequence selected from the group consisting of SEQ ID NOs: 512-515, or an amino acid sequence having at least 90%, 91%, 92%, 93%, 94%, 95%, 96%, 97%, 98%, or 99% sequence identity thereto; and/or (b)) the second targeting moiety comprising a VHH single domain antibody comprising an amino acid sequence selected from the group consisting of SEQ ID NOs: 512-515, or an amino acid sequence having at least 90%, 91%, 92%, 93%, 94%, 95%, 96%, 97%, 98%, or 99% sequence identity thereto.
38. The lipid particle of embodiment 36 or claim 37, wherein (a) the first targeting moiety comprises a VHH comprising the amino acid sequence set forth in any one of SEQ ID NOS: 512-515; and/or (b) the second targeting moiety comprises a VHH comprising the amino acid sequence set forth in any one of SEQ ID NOS: 512-515.
39. The lipid particle of any of embodiments 1-25, and 35-38, wherein the first and second targeting moiety bind to distinct epitopes on CD117.
40. The lipid particle of any of embodiments 35-39, further comprising a second paramyxovirus envelope attachment protein that is a variant paramyxovirus envelope attachment protein comprising one or more amino acid substitutions to reduce the native tropism relative to the wild-type paramyxovirus envelope attachment protein not comprising the one or more amino acid substitutions.
41. The lipid particle of embodiment 40, wherein the second paramyxovirus envelope attachment protein that is a variant paramyxovirus envelope attachment protein comprises one or more amino acid substitutions corresponding to amino acid substitutions selected from the group consisting of E501A, W504A, Q530A and E533A with reference to numbering set forth in SEQ ID NO:1.
42. A lipid particle, comprising: (a) a first retargeted attachment protein comprising (i) a first paramyxovirus envelope attachment protein; and (ii) a first targeting moiety directed to CD8; and a second targeting moiety directed to CD8; and (b) at least one paramyxovirus fusion (F) protein; and the protein in (a), and (b) are exposed on the outside of the lipid bilayer.
43. The lipid particle of embodiment 42, wherein (i) the first targeting moiety is an scFv and comprises an amino acid sequence selected from the group consisting of SEQ ID NOs: 489, 496, 501, or 508 or an amino acid sequence having at least 90%, 91%, 92%, 93%, 94%, 95%, 96%, 97%, 98%, or 99% sequence identity thereto; and/or (ii) the second targeting moiety is an scFv and comprises an amino acid sequence selected from the group consisting of SEQ ID NOs: 489, 496, 501, or 508, or an amino acid sequence having at least 90%, 91%, 92%, 93%, 94%, 95%, 96%, 97%, 98%, or 99% sequence identity thereto.
44. The lipid particle of embodiments 1-25 and 42, wherein (a) the first targeting moiety comprises a CD8 binding agent comprising a VHH single domain antibody comprising a CDR-H1, a CDR-H2, and a CDR-H3 contained within an amino acid sequence of SEQ ID NO: 377; and/or wherein (b) the second targeting moiety comprises a CD8 binding agent comprising a VHH single domain antibody comprising a CDR-H1, a CDR-H2, and a CDR-H3 contained within an amino acid sequence of SEQ ID NO: 377.
45. The lipid particle of embodiment 42 or 44, wherein (a) the first targeting moiety comprises a VHH comprising the amino acid sequence set forth in SEQ ID NOS: 377; and/or (b) the second targeting moiety comprises a VHH comprising the amino acid sequence set forth in SEQ ID NO: 377.
46. The lipid particle of any of embodiments 42-45, wherein (a) the first targeting moiety comprises a VHH comprising the amino acid sequence set forth in SEQ ID NO: 377 or an amino acid sequence having at least 90%, 91%, 92%, 93%, 94%, 95%, 96%, 97%, 98%, or 99% sequence identity thereto; and/or (b) the second targeting moiety is an scFv and comprises an amino acid sequence selected from the group consisting of SEQ ID NO: 501 or an amino acid sequence having at least 90%, 91%, 92%, 93%, 94%, 95%, 96%, 97%, 98%, or 99% sequence identity thereto.
47. The lipid particle of any of embodiments 42-45, wherein (a) the first targeting moiety is an scFv and comprises an amino acid sequence selected from the group consisting of SEQ ID NO: 501 or an amino acid sequence having at least 90%, 91%, 92%, 93%, 94%, 95%, 96%, 97%, 98%, or 99% sequence identity thereto; and (b) the second targeting moiety comprises a VHH comprising the amino acid sequence set forth in SEQ ID NO: 377 or an amino acid sequence having at least 90%, 91%, 92%, 93%, 94%, 95%, 96%, 97%, 98%, or 99% sequence identity thereto.
48. The lipid particle of any of embodiments 1-24, and 42-47, wherein the first and second targeting moiety bind to distinct epitopes on CD8.
49. The lipid particle of any of embodiments 16-48, wherein the distinct epitopes are non-overlapping.
50. The lipid particle of any of embodiments 16-49, wherein the first and second targeting moiety bind to the distinct epitopes in a non-competitive manner.
51. The lipid particle of embodiment 1-50, wherein each of the first targeting moiety and the second targeting moiety are independently selected from the group consisting of an antibody or antigen-binding fragment, a DARPin, an Aptamer, an Affimer, an Affibody, a Knottin, an Avimer, a Monobody, an Anticalin, a Fynomer, and a targeting peptide.
52. The lipid particle of any of embodiments 1-51, wherein the first targeting moiety and the second targeting moiety are independently selected from the group consisting of a single domain antibody or a single chain variable fragment (scFv).
53. The lipid particle of any of embodiments 42-52, further comprising a second paramyxovirus envelope attachment protein that is a variant paramyxovirus envelope attachment protein comprising one or more amino acid substitutions to reduce the native tropism relative to the wild-type paramyxovirus envelope attachment protein not comprising the one or more amino acid substitutions.
54. The lipid particle of embodiment 49, wherein the second paramyxovirus envelope attachment protein that is a variant paramyxovirus envelope attachment protein comprises one or more amino acid substitutions corresponding to amino acid substitutions selected from the group consisting of E501A, W504A, Q530A and E533A with reference to numbering set forth in SEQ ID NO:1.
55. A lipid particle, comprising: (a) a retargeted attachment protein comprising (i) a first paramyxovirus envelope attachment protein; and (ii) a first and second targeting moiety directed to CD133, (b) a second paramyxovirus envelope attachment protein that is a variant paramyxovirus envelope attachment protein comprising one or more amino acid substitutions that reduces the native tropism relative to the wild-type paramyxovirus envelope attachment protein not comprising the one or more amino acid substitutions; and (c) at least one paramyxovirus fusion (F) protein; and wherein the protein in (a), (b) and (c) are exposed on the outside of the lipid bilayer.
56. A lipid particle, comprising: (a) a retargeted attachment protein comprising (i) a first paramyxovirus envelope attachment protein; and (ii) a first and second targeting moiety directed to CD117, (b) a second paramyxovirus envelope attachment protein that is a variant paramyxovirus envelope attachment protein comprising one or more amino acid substitutions that reduces the native tropism relative to the wild-type paramyxovirus envelope attachment protein not comprising the one or more amino acid substitutions; and (c) at least one paramyxovirus fusion (F) protein; and wherein the protein in (a), (b) and (c) are exposed on the outside of the lipid bilayer.
57. A lipid particle, comprising: (a) a retargeted attachment protein comprising (i) a first paramyxovirus envelope attachment protein; and (ii) a first and second targeting moiety directed to CD8, (b) a second paramyxovirus envelope attachment protein that is a variant paramyxovirus envelope attachment protein comprising one or more amino acid substitutions that reduces the native tropism relative to the wild-type paramyxovirus envelope attachment protein not comprising the one or more amino acid substitutions; and (c) at least one paramyxovirus fusion (F) protein; and wherein the protein in (a), (b) and (c) are exposed on the outside of the lipid bilayer.
58. The lipid particle of embodiments 24-57, wherein the first paramyxovirus envelope attachment protein is a variant paramyxovirus envelope attachment protein.
59. The lipid particle of embodiment 58, wherein the variant paramyxovirus envelope attachment protein comprises one or more amino acid substitutions that reduces native tropism relative to the wild-type paramyxovirus envelope attachment protein not comprising the one or more amino acid substitutions.
60. The lipid particle of any of embodiments 25-59, wherein the second paramyxovirus envelope attachment protein is a variant paramyxovirus envelope attachment protein.
61. The lipid particle of embodiment 60, wherein the variant paramyxovirus envelope attachment protein comprises one or more amino acid substitutions that reduces native tropism relative to the wild-type paramyxovirus envelope attachment protein not comprising the one or more amino acid substitutions.
62. The lipid particle of any of embodiments 1-61, wherein the first targeting moiety is selected from the group consisting of a single domain antibody or a single chain variable fragment (scFv).
63. The lipid particle of any of embodiments 1-62, wherein the second targeting moiety is selected from the group consisting of a single domain antibody or a single chain variable fragment (scFv).
64. The lipid particle of embodiments 54 and claim 62, or 63, wherein the single domain antibody is a VHH.
65. The lipid particle of any of embodiments 1-64, wherein the first variant paramyxovirus envelope attachment protein and the second variant paramyxovirus envelope attachment protein are the same.
66. The lipid particle of any of embodiments 1-65, wherein the first paramyxovirus envelope attachment protein and the second paramyxovirus envelope attachment protein are different.
67. The lipid particle of any of embodiments 1-66, wherein the first paramyxovirus envelope attachment protein is an envelope attachment protein from a Nipah virus, Hendra virus, or Measles virus, or is a variant or biologically active portion thereof of any of the foregoing.
68. The lipid particle of any of embodiments 1-67, wherein the first paramyxovirus envelope attachment protein is a wild-type paramyxovirus G protein, H protein or HN protein or is a variant or biologically active portion of any of the foregoing.
69. The lipid particle of embodiment 67 or claim 68, wherein the first paramyxovirus envelope attachment protein is a wild-type Nipah virus G (NiV-G) protein or is a variant or biologically active portion of a NiV-G.
70. The lipid particle of any of embodiments 67-69, wherein the first paramyxovirus envelope attachment protein is a variant NiV-G that is a variant or a biologically active portion of a wild-type NiV-G.
71. The lipid particle of any of embodiments 1-70, wherein the second paramyxovirus envelope attachment protein is an envelope attachment protein from a Nipah virus, Hendra virus, or Measles virus, or is a variant or biologically active portion of any of the foregoing.
72. The lipid particle of any of embodiments 1-71, wherein the second paramyxovirus envelope attachment protein is a wild-type paramyxovirus G protein, H protein or HN protein or is a variant or biologically active portion of any of the foregoing.
73. The lipid particle of embodiment 71 or claim 72, wherein the second paramyxovirus envelope attachment protein is a wild-type Nipah virus G (NiV-G) protein or is a variant or a biologically active portion of a NiV-G.
74. The lipid particle of any of embodiments 1-73, wherein the second paramyxovirus envelope attachment protein is a variant NiV-G that is a variant or a biologically active portion of a wild-type NiV-G.
75. The lipid particle of any of embodiments 3-70, wherein the second paramyxovirus envelope attachment protein is a variant paramyxovirus envelope glycoprotein from a Nipah virus, Hendra virus, or Measles virus or a biologically active portion thereof.
76. The lipid particle of any of embodiments 3-70 and 75, wherein the second paramyxovirus envelope attachment protein is a variant of a wild-type paramyxovirus G protein, H protein or HN protein or a biologically active portion thereof.
77. The lipid particle of embodiment 73 or 74, wherein the variant is a variant NiV-G that is a variant of a wild-type Nipah virus G (NiV-G) protein or a biologically active portion thereof.
78. The lipid particle of embodiment 70, claim 74, or claim 77, wherein the variant NiV-G is truncated by up to 40 contiguous amino acids at or near the N-terminus of the wild-type NiV-G set forth in SEQ ID NO:1.
79. The lipid particle of any of embodiments 70, 74, 77 and 78, wherein the variant NiV-G has a truncation of amino acids 2-34 of the wild-type NiV-G set forth in SEQ ID NO:1.
80. The lipid particle of any of embodiments 70, 73 and 77-79, wherein the variant NiV-G exhibits reduced binding to Ephrin B2 or Ephrin B3.
81. The lipid particle of embodiment 80, wherein the variant NiV-G comprises: one or more amino acid substitutions corresponding to amino acid substitutions selected from the group consisting of E501A, W504A, Q530A and E533A with reference to numbering set forth in SEQ ID NO:1.
82. The lipid particle of embodiment 80 or claim 81, wherein the variant NiV-G comprises amino acid substitutions E501A, W504A, Q530A and E533A with reference to numbering set forth in SEQ ID NO:1.
83. The lipid particle of any of embodiments 70, 74 and 77-79, wherein the variant NiV-G has an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:228.
84. The lipid particle of any of embodiments 70, 74 and 77-79, wherein the variant NiV-G has the amino acid sequence set forth in SEQ ID NO:228.
85. The lipid particle of any of embodiments 1-84, wherein the at least one paramyxovirus fusion (F) protein is an F protein from a henipavirus or is a biologically active portion thereof or variant thereof.
86. The lipid particle of embodiment 85, wherein the henipavirus is a Hendra virus.
87. The lipid particle of embodiment 85, wherein the henipavirus is a Nipah virus.
88. The lipid particle of any of embodiments 1-87, wherein the paramyxovirus F protein is a wild-type NiV-F protein or a variant or a biologically active portion thereof.
89. The lipid particle of any of embodiments 1-88, wherein the paramyxovirus F protein is a variant NiV-F that is a variant or a biologically active portion of a wild-type NiV-F protein.
90. The lipid particle of embodiment 89, wherein the variant NiV-F is truncated by up to 22 contiguous amino acids the at the C-terminus of the wild-type NiV-F set forth in SEQ ID NO:235, optionally not include the initial methionine.
91. The lipid particle of embodiment 89 or claim 90, wherein the variant NiV-F protein is a truncated NiV-F that lacks amino acids 525-546 of SEQ ID NO:235.
92. The lipid particle of any of embodiments 89-91, wherein the variant NiV-F has the amino acid sequence set forth in SEQ ID NO: 227 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:227.
93. The lipid particle of any of embodiments 89-92, wherein the variant NiV-F has the amino acid sequence set forth in SEQ ID NO:227.
94. The lipid particle of any of embodiments 1-93, wherein the paramyxovirus F protein is an F0 precursor or is a proteolytically cleaved form thereof comprising F1 and F2 subunits.
95. The lipid particle of embodiment 94, wherein the proteolytically cleaved form is a cathepsin L cleavage product.
96. The lipid particle of any of embodiments 1-95, wherein the paramyxovirus envelope attachment protein and the first targeting moiety and second targeting moiety are linked via one or more linkers.
97. The lipid particle of embodiment 96, wherein the one or more linkers are one or more peptide linker.
98. The lipid particle of any of embodiments 1-97, wherein the retargeted attachment protein comprises in order: paramyxovirus attachment protein-first linker-first targeting moiety-second linker
second targeting moiety.
99. The lipid particle of embodiment 98, wherein the first linker and/or second linker is independently a peptide linker.
100. The lipid particle of embodiment 99, wherein the first linker and second linker are the same.
101. The lipid particle of embodiment 99, wherein the first linker and second linker are different.
102. The lipid particle of embodiment 97 or claim 99, wherein the peptide linker is 2 to 65 amino acids in length.
103. The lipid particle of any of embodiments 99-102, wherein the peptide linker is a flexible linker that comprises GS, GGS, GGGGS, GGGGGS or combinations thereof.
104. The lipid particle of any of embodiments 99-103, wherein the peptide linker is selected from: (GGS)n, wherein n is 1 to 10; (GGGGS)n, wherein n is 1 to 10; or (GGGGGS)n, wherein n is 1 to 6.
105. The lipid particle of any of embodiments 1-104, wherein the peptide linker is selected from SEQ ID NOs: 589-592.
106. The lipid particle of any of embodiments 1-105, wherein the lipid particle further comprises one or more additional paramyxovirus envelope attachment glycoproteins embedded in the lipid bilayer.
107. The lipid particle of embodiment 106, wherein the one or more additional paramyxovirus envelope attachment glycoproteins is a retargeted attachment protein comprising a paramyxovirus envelope attachment protein and a further targeting moiety.
108. The lipid particle of any of embodiments 1-107, wherein the at least one paramyxovirus fusion (F) protein exhibits fusogenic activity with a target cell upon binding of at least one paramyxovirus envelope attachment protein to the target molecule on the target cell.
109. The lipid particle of any of embodiments 1-108, wherein the lipid particle comprises a viral nucleic acid.
110. The lipid particle of embodiment 109, wherein the viral nucleic acid comprises one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3).
111. The lipid particle of any of embodiments 1-110, wherein the lipid particle is a viral vector.
112. The lipid particle of any of embodiments 1-111, that is a retroviral vector.
113. The lipid particle of any of embodiments 1-111, that is a lentiviral vector.
114. The lipid particle of any of embodiments 1-108 and 111-113, wherein the lipid particle is devoid of viral genomic nucleic acid.
115. The lipid particle of any of embodiments 1-108 and 111-113, wherein the lipid particle is devoid of viral genomic DNA.
116. The lipid particle of any of embodiments 1-110 and 115, that is a viral-like particle.
117. The lipid particle of any of embodiments 1-110, 115 and 116 that is a retroviral-like particle.
118. The lipid particle of any of embodiments 1-110, 115 and 116 that is a lentiviral-like particle.
119. The lipid particle of any of embodiments 1-118, wherein the lipid particle is produced as a preparation with increased titer compared to a reference lipid particle preparation that is similarly produced but with only the first retargeted attachment protein.
120. The lipid particle of any of embodiments 5-119, wherein the lipid particle is produced as a preparation with increased titer compared to a reference lipid particle preparation that is similarly produced but without the second paramyxovirus envelope attachment protein that is a variant paramyxovirus envelope attachment protein comprising one or more substitutions to reduce the native tropism relative to the wild-type paramyxovirus envelope attachment protein not comprising the one or more substitutions.
121. The lipid particle of embodiment 120, wherein the second paramyxovirus envelope attachment protein that is a variant paramyxovirus envelope attachment protein comprises one or more amino acid substitutions corresponding to amino acid substitutions selected from the group consisting of E501A, W504A, Q530A and E533A with reference to numbering set forth in SEQ ID NO:1.
122. The lipid particle of any of embodiments 1-119, wherein the lipid particle is produced in suspension culture as a preparation with increased titer compared to a reference lipid particle preparation that is similarly produced but with only the first retargeted attachment protein.
123. The lipid particle of any of embodiments 120-122, wherein the titer is increased by at or greater than 1.2-fold, 1.3-fold, 1.4-fold, 1.5-fold, 1.6-fold, 1.7-fold, 1.8-fold, 2-fold, 2.5-fold, 3-fold, 4-fold, 5-fold, 6-fold, 7-fold, 8-fold, 9-fold, 10-fold, or more.
124. The lipid particle of any of embodiments 1-123, further comprising an exogenous agent for delivery to a target cell.
125. The lipid particle of embodiment 124, wherein the exogenous agent is present in the lumen.
126. The lipid particle of embodiment 124 or 125, wherein the exogenous agent is a protein or a nucleic acid, optionally wherein the nucleic acid is a DNA or RNA.
127. The lipid particle of any of embodiments 124-126, wherein the exogenous agent is a nucleic acid encoding a cargo for delivery to the target cell.
128. The lipid particle of any of embodiments 124-127, wherein the exogenous agent is or encodes a therapeutic agent, a diagnostic agent or a genome-modifying enzyme.
129. The lipid particle of any of embodiments 124-128, wherein the exogenous agent encodes a membrane protein, optionally wherein the membrane protein is an antigen receptor for targeting cells expressed by or associated with a disease or condition.
130. The lipid particle of embodiment 129, wherein the membrane protein is a chimeric antigen receptor (CAR).
131. The lipid particle of any of embodiments 124-128, wherein the exogenous agent is a nucleic acid comprising a payload gene for correcting a genetic deficiency, optionally a genetic deficiency in the target cell, optionally wherein the genetic deficiency is associated with a liver cell or a hepatocyte.
132. The lipid particle of any of embodiments 124-131, wherein binding of the paramyxovirus envelope attachment protein or biologically active portion thereof to a target molecule expressed on the surface of a target cell mediates fusion of the particle with the target cell and delivery of the exogenous agent to the target cell.
133. The lipid particle of any of embodiments 124-132, wherein at or greater than 10%, 20%, 30%, 40%, 50%, 60% of the target cells are delivered the exogenous agent.
134. The lipid particle of any of embodiments 124-133, wherein delivery of the exogenous cell to the target cell is increased compared to a reference particle preparation that is similarly produced but with only a first retargeted attachment protein.
135. The lipid particle of embodiment 134, wherein the delivery to the target cell is increased by at or greater than 1.2-fold, 1.3-fold, 1.4-fold, 1.5-fold, 1.6-fold, 1.7-fold, 1.8-fold, 2-fold, 2.5-fold, 3-fold, 4-fold, 5-fold, 6-fold, 7-fold, 8-fold, 9-fold, 10-fold, or more.
136. A producer cell comprising (a) a nucleic acid encoding a retargeted attachment protein comprising a paramyxovirus envelope attachment protein linked to (i) a first targeting moiety directed to a first target molecule expressed on the surface of a target cell, and (ii) a second targeting moiety directed to a second target molecule expressed on the surface of a target cell; and (b) a nucleic acid encoding at least one paramyxovirus fusion (F) protein
137. The produce cell of embodiment 136, further comprising a nucleic acid encoding a second paramyxovirus envelope attachment protein that is a variant paramyxovirus envelope attachment protein comprising one or more amino acid substitutions to reduce the native tropism relative to the wild-type paramyxovirus envelope attachment protein not comprising the one or more amino acid substitutions.
138. The producer cell of embodiment 136 or 137, wherein the cell further comprises a viral nucleic acid(s).
139. The producer cell of any of embodiment 138, wherein the viral nucleic acid(s) are lentiviral nucleic acids.
140. The producer cell of any of embodiments 136-139, wherein the cell is a mammalian cell
141. The producer cell of any of embodiments 136-140, wherein the producer cell is selected from the group consisting of CHO cells, BHK cells, MDCK cells, C3H 10T½ cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.
142. The producer cell of any of embodiments 136-141, wherein the producer cell comprises 293T cells.
143. The producer cell of any of embodiments 137-142, wherein the viral nucleic acid(s) lacks one or more genes involved in viral replication.
144. The producer cell of any of embodiments 137-143, wherein the viral nucleic acid comprises a nucleic acid encoding a viral packaging protein selected from one or more of Gag, Pol, Rev and Tat.
145. The producer cell of any of embodiments 138-144, wherein the viral nucleic acid comprises: one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3).
146. A method of making a lipid particle, comprising: a) providing a producer cell of any of embodiments 136-145; b) culturing the cell under conditions that allow for production of the lipid particle, and c) separating, enriching, or purifying the lipid particle from the cell, thereby making the lipid particle.
147. The method of embodiment 146, wherein the lipid particle is a pseudotyped lentiviral vector.
148. A lipid particle produced by the method of embodiment 146 or claim 147.
149. A composition comprising a plurality of lipid particles of any of embodiments 1-135 and 148.
150. The composition of embodiment 149, further comprising a pharmaceutically acceptable carrier.
151. A method of transducing a cell comprising contacting a cell with a lipid particle of any of embodiments 1-135 and 148 or a composition of claim 149 or claim 150.
152. A method of delivering an exogenous agent into a target cell, the method comprising contacting a lipid particle of any of embodiments 124-135 and 148 or a composition of claim 149 or claim 150 with a target cell.
153. The method of embodiment 151 or claim 152, wherein the contacting is in vitro or ex vivo.
154. The method of embodiment 151 or claim 152, wherein the contacting is in vivo in a subject.
155. A method of delivering an exogenous agent to a cell in a subject, the method comprising administering to the subject a lipid particle of any of embodiments 124-135 and 148 or a composition of claim 149 or claim 150.
156. The method of embodiment 155, wherein the exogenous agent is or encodes a therapeutic agent for treating a disease or condition in the subject.
157. A method of treatment, the method comprising administering to a subject a lipid particle of any of embodiments 124-135 and 148 or a composition of claim 149 or claim 150.
158. The method of any of embodiments 152-157, wherein the exogenous agent is or encodes a membrane protein, optionally a chimeric antigen receptor, for targeting an antigen associated with a disease or condition in the subject.
159. The method of any of embodiments 152-157, wherein the exogenous agent is for use in gene therapy to correct a genetic deficiency or replaces a deficient or missing gene in the subject.
160. The method of any of embodiments 155-159, wherein the subject is a human subject.
161. The method of any of embodiments 151-160, wherein the method further comprises administering to the subject one or more agents that stimulate mobilization of bone marrow cells from the bone marrow to the peripheral blood.
162. The method of any of embodiments 151-161, wherein the subject has previously been administered one or more agents that stimulate mobilization of bone marrow cells from the bone marrow to the peripheral blood.
163. The method of embodiment 161 or 162, wherein the one or more agents that stimulate mobilization are selected from the group consisting of stem cell factor (SCF), small molecule VLA-4 inhibitor BI05192, BOP (N-(benzenesulfonyl)-L-prolyl-L-0-(1-pyrrolidinylcarbonyl) tyrosine), heparin, granulocyte colony-stimulating factor (G-CSF), MGTA-145, and plerixafor (AMD3100).
164. The method of any of embodiments 161-163, wherein the one or more agents that stimulate mobilization comprise G-CSF.
165. The method of any of embodiments 161-164, wherein the one or more agents that stimulate mobilization comprise plerixafor.

### VI. EXAMPLES

The following examples are included for illustrative purposes only and are not intended to limit the scope of the invention.

### Example 1 Transduction of CD117 Cells

Studies were carried out to assess methods for retargeting lentiviral vectors (LVs) to specific proteins. In this example, lentiviral vector (LV) was pseudotyped with binders, such as VHH peptides, to re-target the LV with an envelope protein (fusogen) targeting CD117. CD117 is a single pass transmembrane protein (500aa) which is present on at least the surface of hematopoietic stem cells (HSCs), multipotent progenitors (MPPs), common myeloid progenitors (CMPs), and common lymphoid progenitors (CLPs). For targeting to CD117, exemplary CD117 VHH binders were identified in an adherent LV production system for the ability to transduce CD117 overexpressing cells. These binders, set forth in SEQ ID NOS: 512-515, can be used for targeting to CD117.

For targeted delivery of an exemplary exogenous agent, a tandem fusogen containing two VHH binders each directed to CD117 and separated by a linker were produced as a fusion to the C-terminus of the exemplary Nipah G (NiV-G) protein sequence GcΔ34 that was mutated to ablate its native tropism (Bender et al. 2016 PLOS Pathol 12 (6): e1005641). The blinded G comprised point mutations (e.g., amino acid substitutions) E501A, W504A, Q530A and E533A to destroy binding to Ephrin B2/B3, and has the amino acid sequence set forth in SEQ ID NO: 228. In this example, a variety of tandem fusogens were tested that contained a first VHH and a second VHH, as summarized in Table E1. The first and second VHH were linked with a GS linker (e.g., SEQ ID NO:275). The single-binder fusogen containing one VHH binder directed to CD117 was similarly fused to the C-terminus of the GcΔ34 NiV-G as noted below. LV also was produced to contain an exemplary Nipah G (NiV-G) protein sequence GcΔ34 that contained the point mutations E501A, W504A, Q530A and E533A to ablate its native tropism but without fusion with any binder (e.g., no-binder NiV-G or Gm as indicated). The LVs were additionally pseudotyped with the exemplary Nipah F (NiV-F) protein sequence NivFdel22 (SEQ ID NO: 226; or SEQ ID NO:227 without a signal sequence; Bender et al. 2016 PLOS).

E1: Exemplary CD117 Tandem and Single Fusogens

| | | |
|---|---|---|
| Fusogen | First VHH | Second VHH |

CD117 Tandem

| | | |
|---|---|---|
| CD117_A | CD117_B2 | CD117_B4 |
| (SEQ ID NO 575) | (SEQ ID NO 513) | (SEQ ID NO 515) |
| CD117_B | CD117_B2 | CD117_B1 |
| (SEQ ID NO 576) | (SEQ ID NO 513) | (SEQ ID NO 512) |
| CD117_C | CD117_B4 | CD117_B1 |
| (SEQ ID NO 577) | (SEQ ID NO 515) | (SEQ ID NO 512) |
| CD117_D | CD117_B1 | CD117_B4 |
| (SEQ ID NO 578) | (SEQ ID NO 512) | (SEQ ID NO 515) |
| CD117_E | CD117_B1 | CD117_B1 |
| (SEQ ID NO 579) | (SEQ ID NO 512) | (SEQ ID NO 512) |
| CD117_F | CD117_B4 | CD117_B4 |
| (SEQ ID NO 580) | (SEQ ID NO 515) | (SEQ ID NO 515) |
| CD117_G | CD117_B2 | CD117_B2 |
| (SEQ ID NO 581) | (SEQ ID NO 513) | (SEQ ID NO 513) |
| CD117_H | CD117_B3 | CD117_B4 |
| (SEQ ID NO 586) | (SEQ ID NO 514) | (SEQ ID NO 515) |
| CD117_I | CD117_B1 | CD117_B3 |
| (SEQ ID NO 587) | (SEQ ID NO 512) | (SEQ ID NO 514) |
| CD117_J | CD117_B2 | CD117_B3 |
| (SEQ ID NO 588) | (SEQ ID NO 513) | (SEQ ID NO 514) |

CD117 Single Binder

| | |
|---|---|
| CD117_B1 | — |
| (SEQ ID NO 512) | |
| CD117_B2 | — |
| (SEQ ID NO 513) | |
| CD117_B3 | — |
| (SEQ ID NO 514) | |
| CD117_B4 | — |
| (SEQ ID NO 515) | |

For producing the lentiviral vector, plasmids encoding either the tandem re-targeted NiV-G fusion protein or the single re-targeted NiV-G fusion protein, the no-binder NiV-G protein, and the NiV-F protein were transfected into the same population of HEK 293 cells. The cells also were transfected with packaging plasmid for viral vector production (e.g. gag/pol, rev), and a transfer plasmid encoding enhanced green fluorescent protein (eGFP). Following viral vector production, the cell culture was centrifuged to pellet the cells and the supernatant containing crude virus was collected.

The exemplary CD117 tandem VHH binders and single VHH binders were assessed for titer in a CD117-overexpressing cell line (FIG. 1A). Highest functional titer was observed for LV pseudotyped with a fusion NiV-G comprising tandem binders. This was observed to be correlated with a functional crude titers within 106 when used to transduce a CD117 overexpressing cell line.

In a complementary experiment, LV pseudotyped with a re-targeted tandem fusogen targeting CD117 and a NiV-F protein as described above was produced in SCC3687 cells, a squamous cell carcinoma line. LV was concentrated 100×via ultracentrifuge and tested for titer on both CD117-overexpressing cells (FIG. 1B) and CD34+ cells (FIG. 1C). It was observed that some tandem binders are particularly potent at transducing CD34+ cells. For example, exemplary tandem fusogen CD117_A (SEQ ID NO 575) showed a twofold improvement in titer on CD117 overexpressing cells and nearly 2-log improvement with CD34+ cells.

Two exemplary tandem binders were next assessed for primary cell transduction in comparison with a single binder also targeted to CD117 (FIG. 1D). It was observed that the candidate CD117 tandem binder CD117_A (SEQ ID NO 575) showed potency when transducing unstimulated CD34+ primary cells. In contrast, the single binder showed little to no ability to transduce CD34+ primary cells despite high titers on the CD117 overexpressing cell line.

These results support that pseudotyping lipid particles, such as LVs, with a tandem binder fused to a viral attachment protein substantially improves targets of the LV to a desired target cell.

### Example 2 Transduction of CD133+CD34+ Cells

Similarly to the experiments described above, lentiviral vector (LV) was pseudotyped with binders, such as scFv peptides, to re-target the LV with an envelope protein (fusogen) targeting CD133. For targeting to CD133, exemplary CD133 scFv binders as set forth in SEQ ID NOS: 534, 543, 516, 525, 552, can be used.

For targeted delivery of an exemplary exogenous agent, two tandem fusogens containing two scFv binders each directed to CD133 and separated by a linker were produced as a fusion to the C-terminus of the exemplary Nipah G (NiV-G) protein sequence GcΔ34 that was mutated to ablate its native tropism (Bender et al. 2016 PLOS Pathol 12 (6): e1005641). The blinded G comprised point mutations E501A, W504A, Q530A and E533A to destroy binding to Ephrin B2/B3, and has the amino acid sequence set forth in SEQ ID NO: 228. In this example, both tandem fusogens were tested that contained a first scFv and a second scFv, as summarized in Table E2. The first and second scFv were linked with a GS linker (e.g., SEQ ID NO:275). The single-binder fusogen containing one scFv binder directed to CD133 was similarly fused to the C-terminus of the GcΔ34 NiV-G as noted below. LV also was produced to contain an exemplary Nipah G (NiV-G) protein sequence GcΔ34 that contained the point mutations E501A, W504A, Q530A and E533A to ablate its native tropism but without fusion with any binder (e.g., no-binder NiV-G or Gm as indicated). The LVs were additionally pseudotyped with the exemplary Nipah F (NiV-F) protein sequence NivFdel22 (SEQ ID NO:226; or SEQ ID NO:227 without a signal sequence; Bender et al. 2016 PLOS).

E2: Exemplary CD133 Tandem and Single Fusogens

| | | |
|---|---|---|
| Fusogen | First VHH | Second VHH |

CD133 Tandem

| | | |
|---|---|---|
| CD133_A | CD133_B3 | CD133_B4 |
| (SEQ ID NO 582) | (SEQ ID NO 517) | (SEQ ID NO 526) |
| CD133_B | CD133_B4 | CD133_B3 |
| (SEQ ID NO 583) | (SEQ ID NO 526) | (SEQ ID NO 517) |

CD133 Single Binder

| | |
|---|---|
| CD133_B3 | — |
| (SEQ ID NO 517) | |
| CD133_B4 | — |
| (SEQ ID NO 526) | |

For producing the lentiviral vector, plasmids encoding either the tandem re-targeted NiV-G fusion protein or the single re-targeted NiV-G fusion protein, the no-binder NiV-G protein, and the NiV-F protein were transfected into the same population of HEK 293 cells. The cells also were transfected with packaging plasmid for viral vector production (e.g. gag/pol, rev), and a transfer plasmid encoding enhanced green fluorescent protein (eGFP). Following viral vector production, the cell culture was centrifuged to pellet the cells and the supernatant containing crude virus was collected.

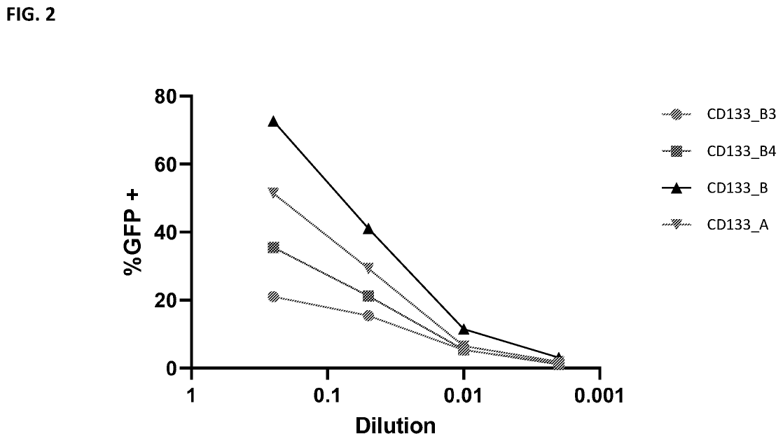

Two exemplary CD133 tandem binders were tested against each construct as a single scFv binder for transduction of CD34+ primary cells (FIG. 2). It was observed that placing the exemplary CD133 binders in tandem resulted in higher transduction of CD34+ cells as compared with single binders.

These results support that pseudotyping lipid particles, such as LVs, with a tandem binder fused to a viral attachment protein substantially improves targets of the LV to a desired target cell.

### Example 3 Transduction of CD8+ T Cells

Similarly to the experiments described above, lentiviral vector (LV) was pseudotyped with binders, such as scFv or VHH peptides, to re-target the LV with an envelope protein (fusogen) targeting CD8. For targeting to CD8, exemplary CD8 binders as set forth in SEQ ID NOS: 377, 489, 496, 501, or 508 can be used.

For targeted delivery of an exemplary exogenous agent, two tandem fusogens containing two binders each directed to CD8 and separated by a linker were produced as a fusion to the C-terminus of the exemplary Nipah G (NiV-G) protein sequence GcΔ34 that was mutated to ablate its native tropism (Bender et al. 2016 PLOS Pathol 12 (6): e1005641). The blinded G comprised point mutations E501A, W504A, Q530A and E533A to destroy binding to Ephrin B2/B3, and has the amino acid sequence set forth in SEQ ID NO: 228. In this example, the tandem fusogens were tested that contained a first binder and a second binder, as summarized in Table E3. The first and second binders were linked with a GS linker (e.g., SEQ ID NO:275). The single-binder fusogen containing one scFv or VHH binder directed to CD8 was similarly fused to the C-terminus of the GcΔ34 NiV-G as noted below. LV also was produced to contain an exemplary Nipah G (NiV-G) protein sequence GcΔ34 that contained the point mutations E501A, W504A, Q530A and E533A to ablate its native tropism but without fusion with any binder (e.g., no-binder NiV-G or Gm as indicated). The LVs were additionally pseudotyped with the exemplary Nipah F (NiV-F) protein sequence NivFdel22 (SEQ ID NO:226; or SEQ ID NO:227 without a signal sequence; Bender et al. 2016 PLOS).

E3: Exemplary CD8 Tandem and Single Fusogens

| | | |
|---|---|---|
| Fusogen | First VHH/Binder | Second VHH/Binder |

CD8 Tandem

| | | |
|---|---|---|
| CD8_A | CD8_B1 | CD8_B2 |
| (SEQ ID NO 584) | (SEQ ID NO 377) | (SEQ ID NO 501) |
| CD8_B | CD8_B2 | CD8_B1 |
| (SEQ ID NO 585) | (SEQ ID NO 501) | (SEQ ID NO 377) |

CD8 Single Binder

| | |
|---|---|
| CD8_B1 | — |
| (SEQ ID NO 377) | |
| CD8_B2 | — |
| (SEQ ID NO 501) | |

For producing the lentiviral vector, plasmids encoding either the tandem re-targeted NiV-G fusion protein or the single re-targeted NiV-G fusion protein, the no-binder (blinded) NiV-G protein, and the NiV-F protein were transfected into the same population of HEK 293 cells. The cells also were transfected with packaging plasmid for viral vector production (e.g. gag/pol, rev), and a transfer plasmid encoding enhanced green fluorescent protein (eGFP). Following viral vector production, the cell culture was centrifuged to pellet the cells and the supernatant containing crude virus was collected.

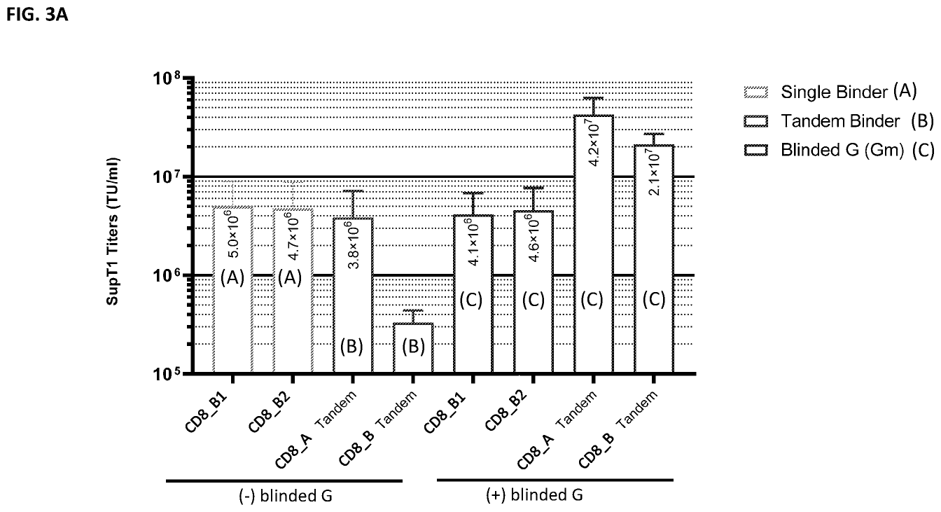

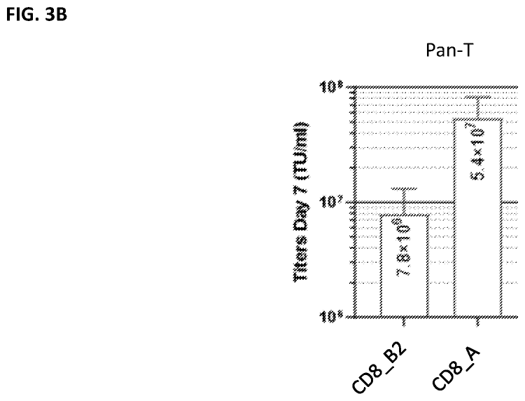

Two exemplary CD8 tandem binders, CD8_A and CD8_B, were tested against each binder construct as a single VHH or scFv binder for transduction of SupT1 T cells (FIG. 3A). It was observed that placing the exemplary CD8 binders in tandem resulted in higher transduction of T cells as compared with single binders. It was observed that the LV pseudotyped with the tandem CD8 NiV-G binder showed a 10-fold increase in titer when expressed on the vector with the blinded G.

An exemplary CD8 tandem binder CD8_A was tested against a single binder construct (CD8_B2) for transduction of resting Pan-T cells. For this experiment, frozen Pan-T cells were thawed and plated in IL-2 (10 mg/mL) (or IL-7) and expanded. Following expansion, 80,000-100,000 cells were plated per well of a 96-well plate in 100 μL of Immunocult medium+pen/strep+IL-2. About 100 μL of lentiviral preps were added to the cells, and the 96-well plate was spun at 10000 g for 90 minutes at room temperature. Cells were then activated using CD3/CD28 activator beads (25 μL/1E6cells) and IL7. After an incubation at 37 C, cells were harvested for staining and/or VCN analysis to determine titers. FIG. 3B shows Pan-T titers for single binder (CD8_B2) and tandem binder (CD8_A). It was observed that including the no-binder NiV-G along with the re-targeted Ni-G during production led to increased titers for at least the exemplary CD8 binders in tandem. It was also observed that tandem binder CD8_A (SEQ ID NO: 584 comprising the CD8_B1 binder (SEQ ID NO: 377) and the CD8_B2 binder (SEQ ID NO: 501)) had a 5-fold increase compared to single binder CD8_B2.

These results support that pseudotyping lipid particles, such as LVs, with a tandem binder that is either of an scFv or VHH, or a combination thereof, fused to a viral attachment protein substantially improves targets of the LV to a desired target cell.

### Example 4: Selective Modulation of CD34+ Cells In Vivo

In a complementary set of experiments similar to Example 1 above, lentiviral vector (LV) was pseudotyped with binders, such as VHH peptides, to re-target the LV with an envelope protein (fusogen) targeting CD117. These binders, e.g., set forth in SEQ ID NOS: 512-515, can be used for targeting to CD117.

For targeted delivery of an exemplary exogenous agent, two tandem fusogens containing two different VHH binders each directed to CD117 and separated by a linker were produced as a fusion to the C-terminus of the exemplary Nipah G (NiV-G) protein sequence GcΔ34 that was mutated to ablate its native tropism (Bender et al. 2016 PLOS Pathol 12 (6): e1005641). The blinded G comprised point mutations E501A, W504A, Q530A and E533A to destroy binding to Ephrin B2/B3 and has the amino acid sequence set forth in SEQ ID NO: 228. The first and second VHH were linked with a GS linker (e.g., SEQ ID NO:275). LV also was produced to contain an exemplary Nipah G (NiV-G) protein sequence GcΔ34 that contained the point mutations E501A, W504A, Q530A and E533A to ablate its native tropism but without fusion with any binder (e.g., no-binder NiV-G or Gm as indicated). The LVs were additionally pseudotyped with the exemplary Nipah F (NiV-F) protein sequence NivFdel22 (SEQ ID NO: 226; or SEQ ID NO:227 without a signal sequence; Bender et al. 2016 PLOS). Control BaEVTR LV was produced using the BaEVTR envelope.

For producing the lentiviral vector, plasmids encoding either the tandem re-targeted NiV-G fusion protein or the single re-targeted NiV-G fusion protein, the no-binder NiV-G protein, and the NiV-F protein were transfected into the same population of HEK 293 cells. The cells also were transfected with packaging plasmid for viral vector production (e.g. gag/pol, rev), and a transfer plasmid encoding a gene for GFP. Following viral vector production, the cell culture was centrifuged to pellet the cells and the supernatant containing crude virus was collected.

The ability of a lentiviral vector pseudotyped with a tandem fusogen to specifically target a minor population of cells in the bone marrow was studied. To assess in vivo delivery, human CD34+ cells (huCD34+) were infused into NBSGW mice (NOD.Cg-KitW-41J Tyr+Prkdescid Il2rgtm1Wjl/ThomJ). Following 84 days of maintenance, GFP expressing lentiviral vector were delivered intravenously. Mice were sacrificed on Day 96, and cells of the bone marrow were assessed for GFP expression.

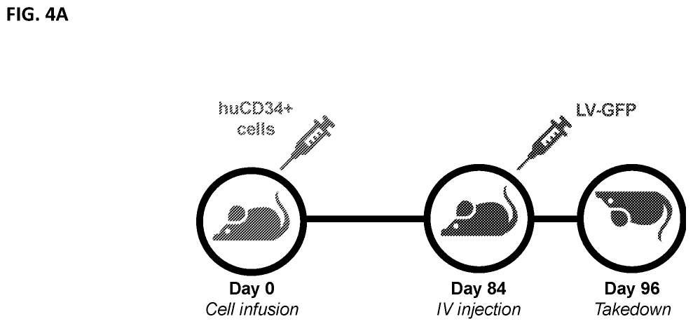

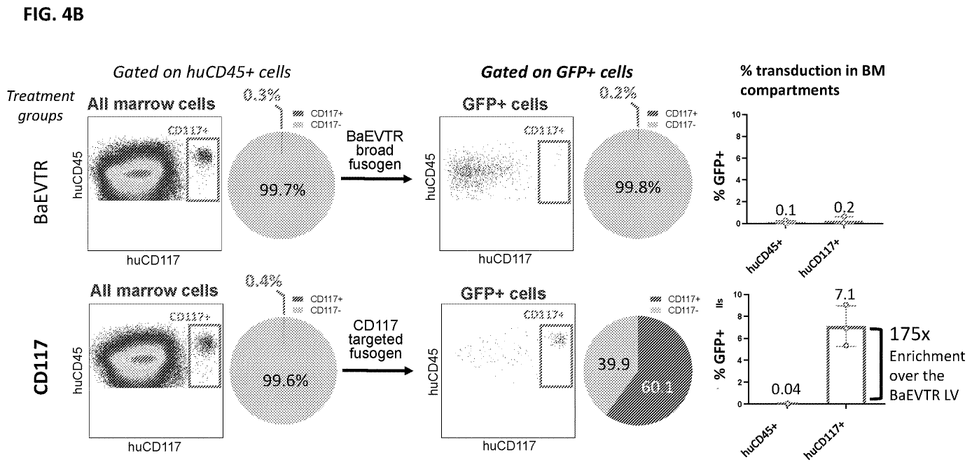

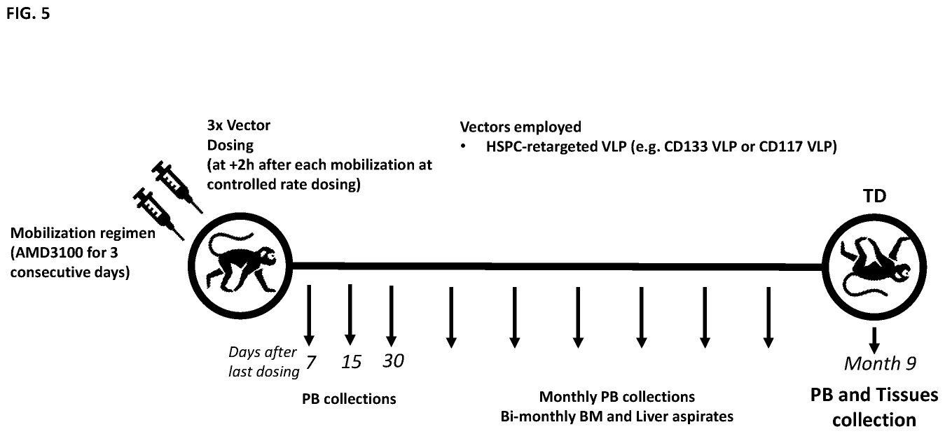

Transduction efficiency for mice which were equivalently dosed with the CD117 LV was compared to the BaEVTR LV as shown in FIG. 4A. It was observed that, when gating on GFP+ cells in the bone marrow (BM), only 0.2% of these cells were CD117+ when using BaEVTR LV. This stands in contrast to the 60% observed following administration of the CD117 LV (FIG. 4B). Similarly, when measuring % GFP on huCD45+ cells, no major difference was observed between the two vectors at equivalent doses. However, gating on the target CD117+ population instead yielded a 175-fold higher targeting of this population with CD117 LV over BaEVTR LV (FIG. 4B).

These results demonstrate that an exemplary lentiviral vector pseudotyped with a tandem fusogen directed to CD117 is highly efficient in editing of CD34+ cells in vivo, as well as demonstrating a 178-fold targeting selectivity of the vector for target cells in vivo. Without wishing to be bound by theory, CD117's localization to the bone marrow likely explains the increased selectivity in vivo. Taken together, these data reflect a high specificity for the retargeted CD117 LV and demonstrate the ability of the retargeted CD117 LV to reach the cognate target cell population all while largely avoiding transduction of off-target cells.

### Example 5: Gene Editing In Vivo in Non-Human Primate Model

This example sets forth an exemplary design to assess various fusogen-payload combinations for administration in a Non-Human Primate (NHP). Virus-like particles (VLPs) are produced with tandem Nipah virus (NiV) fusogens retargeted to HSPC with specific binders against CD133 and/or CD117. These binder-retargeted Nipah G fusogens comprise two HSPC binding moieties linked in tandem to the exemplary NiV-G sequence GcΔ34 set forth in SEQ ID NO:228. The VLPs that are pseudotyped with the tandem fusogens are in some embodiments further pseudotyped with a blinded NiV-G that comprise amino acid substitutions E501A, W504A, Q530A and E533A to ablate binding to Ephrin B2/B3 (e.g. set forth in SEQ ID NO:228) but that is not retargeted by fusion with a binder sequence or tandem binder sequences. For NiV fusogens, VLPs are produced with the Nipah F protein and the binder-retargeted tandem Nipah G protein (see US 2019/0144885, incorporated by reference herein e.g. NivFdel22 (SEQ ID NO:226; or SEQ ID NO:227, which does not have a signal sequence; Bender et al. 2016 PLOS)) expressed on the lipid envelope.

The VLPs are also packaged with a gene editing payload. Following VLP production, the cell culture is centrifuged to pellet the cells and the supernatant containing crude virus is collected.

A primary fusogen is selected based in part on overall VLP performance as measured by percent of long-term editing (i.e., edits resulting from the payload) in peripheral blood myeloid cells. A second fusogen is selected based in part on HSPC specific tropism as measured, for example, by percent of editing (i.e., edits resulting from the payload) in the various mature hematopoietic lineages derived from the HSPCs or in other cell lines vs the target HSPC population.

A payload (e.g., such as nuclease-based editor or base editing-based editor) is selected based on performance in resting NHPCD34+ cells in vitro as per OTA PCR based analysis. Performance can be either reporter-based or a functional readout (such as change in mRNA or protein levels in a biological sample). A genomic target is selected that is characterized by a “neutral” locus or a “therapeutic” locus homologous to a potential therapeutic target for a disease indication (e.g. BCL11A).

A model study design is illustrated in FIG. 5. NHP animals are first subject to a mobilization regimen as described above. Briefly, AMD3100 is administered for 3 consecutive days wherein VLP is administered roughly 2 hours after each mobilization at a controlled rate dosing. Without wishing to be bound by theory, mobilization of cells from the bone marrow to the peripheral blood is generally at its peak 1.5-3 hours post AMD treatment. Peripheral blood is collected throughout the study period, while bone marrow is collected through aspirates bi-monthly. Additional needle aspirates of the liver are performed to assess off-target editing as a function of safety and biodistribution. Animals are sacrificed at 9 months post vector dose for assessing permanent gene modification of the long-term HSC in the bone marrow and potential editing in off-target tissues.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

VII. SEQUENCES

| SEQ | | |
|---|---|---|
| ID NO | SEQUENCE | ANNOTATION |
| 1 | MPAENKKVRFENTTSDKGKIPSKVIKSYYGTMDIKKIN | NiVG protein attachment |
| | EGLLDSKILSAFNTVIALLGSIVIIVMNIMIIQNYTRSTD | glycoprotein (602 aa) |
| | NQAVIKDALQGIQQQIKGLADKIGTEIGPKVSLIDTSST | Uniprot Q9IH62 |
| | ITIPANIGLLGSKISQSTASINENVNEKCKFTLPPLKIHE | Cytoplasmic tail 1-49 |
| | CNISCPNPLPFREYRPQTEGVSNLVGLPNNICLQKTSN | Transmembrane 50-70 |
| | QILKPKLISYTLPVVGQSGTCITDPLLAMDEGYFAYSH | Ectodomain 71-602; Stalk |
| | LERIGSCSRGVSKQRIIGVGEVLDRGDEVPSLFMTNVW | domain 71-187; head domain |
| | TPPNPNTVYHCSAVYNNEFYYVLCAVSTVGDPILNST | 188-602) |
| | YWSGSLMMTRLAVKPKSNGGGYNQHQLALRSIEKGR | |
| | YDKVMPYGPSGIKQGDTLYFPAVGFLVRTEFKYNDSN | |
| | CPITKCQYSKPENCRLSMGIRPNSHYILRSGLLKYNLS | |
| | DGENPKVVFIEISDQRLSIGSPSKIYDSLGQPVFYQASFS | |
| | WDTMIKFGDVLTVNPLVVNWRNNTVISRPGQSQCPRF | |
| | NTCPEICWEGVYNDAFLIDRINWISAGVFLDSNQTAEN | |
| | PVFTVFKDNEILYRAQLASEDTNAQKTITNCFLLKNKI | |
| | WCISLVEIYDTGDNVIRPKLFAVKIPEQCT | |
| 2 | ILSAFNTVIALLGSIVIIVMNIMIIQNYTRSTDNQAVIKD | NiV-G backbone ACT |
| | ALQGIQQQIKGLADKIGTEIGPKVSLIDTSSTITIPANIGL | |
| | LGSKISQSTASINENVNEKCKFTLPPLKIHECNISCPNPL | |
| | PFREYRPQTEGVSNLVGLPNNICLQKTSNQILKPKLISY | |
| | TLPVVGQSGTCITDPLLAMDEGYFAYSHLERIGSCSRG | |
| | VSKQRIIGVGEVLDRGDEVPSLFMTNVWTPPNPNTVY | |
| | HCSAVYNNEFYYVLCAVSTVGDPILNSTYWSGSLMM | |
| | TRLAVKPKSNGGGYNQHQLALRSIEKGRYDKVMPYG | |
| | PSGIKQGDTLYFPAVGFLVRTEFKYNDSNCPITKCQYS | |
| | KPENCRLSMGIRPNSHYILRSGLLKYNLSDGENPKVVF | |
| | IEISDQRLSIGSPSKIYDSLGQPVFYQASFSWDTMIKFG | |
| | DVLTVNPLVVNWRNNTVISRPGQSQCPRFNTCPEICW | |
| | EGVYNDAFLIDRINWISAGVFLDSNQTAENPVFTVFKD | |
| | NEILYRAQLASEDTNAQKTITNCFLLKNKIWCISLVEIY | |
| | DTGDNVIRPKLFAVKIPEQCT | |
| 3 | ILSAFNTVIALLGSIVIIVMNIMIIQNYTRSTDNQAVIKD | NiV-G backbone ACT and |
| | ALQGIQQQIKGLADKIGTEIGPKVSLIDTSSTITIPANIGL | mutated |
| | LGSKISQSTASINENVNEKCKFTLPPLKIHECNISCPNPL | (E501 A, W504A, Q530A, |
| | PFREYRPQTEGVSNLVGLPNNICLQKTSNQILKPKLISY | E533A) |
| | TLPVVGQSGTCITDPLLAMDEGYFAYSHLERIGSCSRG | |
| | VSKQRIIGVGEVLDRGDEVPSLFMTNVWTPPNPNTVY | |
| | HCSAVYNNEFYYVLCAVSTVGDPILNSTYWSGSLMM | |
| | TRLAVKPKSNGGGYNQHQLALRSIEKGRYDKVMPYG | |
| | PSGIKQGDTLYFPAVGFLVRTEFKYNDSNCPITKCQYS | |
| | KPENCRLSMGIRPNSHYILRSGLLKYNLSDGENPKVVF | |
| | IEISDQRLSIGSPSKIYDSLGQPVFYQASFSWDTMIKFG | |
| | DVLTVNPLVVNWRNNTVISRPGQSQCPRFNTCPAICA | |
| | EGVYNDAFLIDRINWISAGVFLDSNATAANPVFTVFK | |
| | DNEILYRAQLASEDTNAQKTITNCFLLKNKIWCISLVEI | |
| | YDTGDNVIRPKLFAVKIPEQCT | |
| 4 | MPAENKKVRFENTTSDKGKIPSKVIKSYYGTMDIKKIN | NivG CT_45_FL |
| | EGLLDSK | |
| 5 | MPAENKKVRFENTTSDKGKIPSKVIKSYYGTMDIKKIN | NiVG with NiVG |
| | EGLLDSKILSAFNTVIALLGSIVIIVMNIMIIQNYTRSTD | CT_45_FL, mutated (E501 |
| | NQAVIKDALQGIQQQIKGLADKIGTEIGPKVSLIDTSST | A, W504A, Q530A, E533A) |
| | ITIPANIGLLGSKISQSTASINENVNEKCKFTLPPLKIHE | |
| | CNISCPNPLPFREYRPQTEGVSNLVGLPNNICLQKTSN | |
| | QILKPKLISYTLPVVGQSGTCITDPLLAMDEGYFAYSH | |
| | LERIGSCSRGVSKQRIIGVGEVLDRGDEVPSLFMTNVW | |
| | TPPNPNTVYHCSAVYNNEFYYVLCAVSTVGDPILNST | |
| | YWSGSLMMTRLAVKPKSNGGGYNQHQLALRSIEKGR | |
| | YDKVMPYGPSGIKQGDTLYFPAVGFLVRTEFKYNDSN | |
| | CPITKCQYSKPENCRLSMGIRPNSHYILRSGLLKYNLS | |
| | DGENPKVVFIEISDQRLSIGSPSKIYDSLGQPVFYQASFS | |
| | WDTMIKFGDVLTVNPLVVNWRNNTVISRPGQSQCPRF | |
| | NTCPAICAEGVYNDAFLIDRINWISAGVFLDSNATAAN | |
| | PVFTVFKDNEILYRAQLASEDTNAQKTITNCFLLKNKI | |
| | WCISLVEIYDTGDNVIRPKLFAVKIPEQCT | |
| 6 | MLDSK | NivG_CT_05 cytoplasmic |
| | | tail |
| 7 | MLLDSK | NivG_CT_06 cytoplasmic |
| | | tail |
| 8 | MGLLDSK | NivG_CT_07 cytoplasmic |
| | | tail |
| 9 | MEGLLDSK | NivG_CT_08 cytoplasmic |
| | | tail |
| 10 | MNEGLLDSK | NivG_CT_09 cytoplasmic |
| | | tail |
| 11 | MINEGLLDSK | NivG_CT_10 cytoplasmic |
| | | tail |
| 12 | MKINEGLLDSK | NivG_CT_11 cytoplasmic |
| | | tail |
| 13 | MIKKINEGLLDSK | NivG_CT_13 cytoplasmic |
| | | tail |
| 14 | MDIKKINEGLLDSK | NivG_CT_14 cytoplasmic |
| | | tail |
| 15 | MMDIKKINEGLLDSK | NivG_CT_15 cytoplasmic |
| | | tail |
| 16 | MTMDIKKINEGLLDSK | NivG_CT_16 cytoplasmic |
| | | tail |
| 17 | MGTMDIKKINEGLLDSK | NivG_CT_17 cytoplasmic |
| | | tail |
| 18 | MYGTMDIKKINEGLLDSK | NivG_CT_18 cytoplasmic |
| | | tail |
| 19 | MYYGTMDIKKINEGLLDSK | NivG_CT_19 cytoplasmic |
| | | tail |
| 20 | MSYYGTMDIKKINEGLLDSK | NivG_CT_20 cytoplasmic |
| | | tail |
| 21 | MKSYYGTMDIKKINEGLLDSK | NivG_CT_21 cytoplasmic |
| | | tail |
| 22 | MIKSYYGTMDIKKINEGLLDSK | NivG_CT_22 cytoplasmic |
| | | tail |
| 23 | MVIKSYYGTMDIKKINEGLLDSK | NivG_CT_23 cytoplasmic |
| | | tail |
| 24 | MKVIKSYYGTMDIKKINEGLLDSK | NivG_CT_24 cytoplasmic |
| | | tail |
| 25 | MSKVIKSYYGTMDIKKINEGLLDSK | NivG_CT_25 cytoplasmic |
| | | tail |
| 26 | MKGKNPSKVIKSYYGTMDIKKINEGLLDSK | NivG_CT_30 cytoplasmic |
| | | tail |
| 27 | MNTTSDKGKNPSKVIKSYYGTMDIKKINEGLLDSK | NivG_CT_35 cytoplasmic |
| | | tail |
| 28 | MKVRFENTTSDKGKNPSKVIKSYYGTMDIKKINEGLL | NivG_CT_40 cytoplasmic |
| | DSK | tail |
| 29 | MIIKSYYGTMDIKKINEGLLDSK | NivG_CSUR38_CT_23 |
| | | cytoplasmic tail |
| 30 | MGKIPSKIIKSYYGTMDIKKINEGLLDSK | NivG_CSUR38_CT_29 |
| | | cytoplasmic tail |
| 31 | MNTTSDKGKIPSKIIKSYYGTMDIKKINEGLLDSK | NivG_CSUR38_CT_35 |
| | | cytoplasmic tail |
| 32 | MSKKVRFENTTSDKGKIPSKIIKSYYGTMDIKKINEGL | NivG_CSUR38_CT_42 |
| | LDSK | cytoplasmic tail |
| 33 | MPAESKKVRFENTTSDKGKIPSKIIKSYYGTMDIKKINE | NivG_CSUR38_CT_45_FL |
| | GLLDSK | cytoplasmic tail |
| 34 | MNPSKVIKSYYGTMDIKKINEGLLDSK | NivG_VRI-0626_CT_27 |
| | | cytoplasmic tail |
| 35 | MDKGKNPSKVIKSYYGTMDIKKINEGLLDSK | NivG_VRI-0626_CT_31 |
| | | cytoplasmic tail |
| 36 | MNTTSDKGKNPSKVIKSYYGTMDIKKINEGLLDSK | NivG_VRI-0626_CT_35 |
| 37 | MKVRFENTTSDKGKNPSKVIKSYYGTMDIKKINEGLL | NivG_VRI-0626_CT_40 |
| | DSK | cytoplasmic tail |
| 38 | MPAENKKVRFENTTSDKGKNPSKVIKSYYGTMDIKKI | NivG_VRI- |
| | NEGLLDSK | 0626_CT_45_FL |
| | | cytoplasmic tail |
| 39 | MKKINEGLLDSK | NivG_CT_12 cytoplasmic |
| | | tail |
| 40 | MDGLLDSK | HevG_CT_08 |
| 41 | MNDGLLDSK | HevG_CT_09 |
| 42 | MINDGLLDSK | HevG_CT_10 |
| 43 | MKINDGLLDSK | HevG_CT_11 |
| 44 | MKKINDGLLDSK | HevG_CT_12 |
| 45 | MIKKINDGLLDSK | HevG_CT_13 |
| 46 | MDIKKINDGLLDSK | HevG_CT_14 |
| 47 | MMDIKKINDGLLDSK | HevG_CT_15 |
| 48 | MTMDIKKINDGLLDSK | HevG_CT_16 |
| 49 | MGTMDIKKINDGLLDSK | HevG_CT_17 |
| 50 | MYGTMDIKKINDGLLDSK | HevG_CT_18 |
| 51 | MYYGTMDIKKINDGLLDSK | HevG_CT_19 |
| 52 | MNYYGTMDIKKINDGLLDSK | HevG_CT_20 |
| 53 | MKNYYGTMDIKKINDGLLDSK | HevG_CT_21 |
| 54 | MIKNYYGTMDIKKINDGLLDSK | HevG_CT_22 |
| 55 | MVIKNYYGTMDIKKINDGLLDSK | HevG_CT_23 |
| 56 | MKVIKNYYGTMDIKKINDGLLDSK | HevG_CT_24 |
| 57 | MMADSKLVSLNNNLSGKIKDQGKVIKNYYGTMDIKK | HevG_CT_45_FL |
| | INDGLLDSK | |
| 58 | MQNDHY | BatPV_G_CT_6 |
| 59 | MNQNDHY | BatPV_G_CT_7 |
| 60 | MKNQNDHY | BatPV_G_CT_8 |
| 61 | MQKNQNDHY | BatPV_G_CT_9 |
| 62 | MKQKNQNDHY | BatPV_G_CT_10 |
| 63 | MKKQKNQNDHY | BatPV_G_CT_11 |
| 64 | MWKKQKNQNDHY | BatPV_G_CT_12 |
| 65 | MNWKKQKNQNDHY | BatPV_G_CT_13 |
| 66 | MRNWKKQKNQNDHY | BatPV_G_CT_14 |
| 67 | MERNWKKQKNQNDHY | BatPV_G_CT_15 |
| 68 | MSERNWKKQKNQNDHY | BatPV_G_CT_16 |
| 69 | MHSERNWKKQKNQNDHY | BatPV_G_CT_17 |
| 70 | MSHSERNWKKQKNQNDHY | BatPV_G_CT_18 |
| 71 | MGSHSERNWKKQKNQNDHY | BatPV_G_CT_19 |
| 72 | MLGSHSERNWKKQKNQNDHY | BatPV_G_CT_20 |
| 73 | MGLGSHSERNWKKQKNQNDHY | BatPV_G_CT_21 |
| 74 | MFGLGSHSERNWKKQKNQNDHY | BatPV_G_CT_22 |
| 75 | MYFGLGSHSERNWKKQKNQNDHY | BatPV_G_CT_23 |
| 76 | MGYFGLGSHSERNWKKQKNQNDHY | BatPV_G_CT_24 |
| 77 | MKITKQGYFGLGSHSERNWKKQKNQNDHY | BatPV_G_CT_29 |
| 78 | MPQKTVEFINMNSPLERGVSTLSDKKTLNQSKITKQG | BatPV_G_CT_59_FL |
| | YFGLGSHSERNWKKQKNQNDHY | |
| 79 | MLHDIK | CedarV_G_CT_6 |
| 80 | MSLHDIK | CedarV_G_CT_7 |
| 81 | MESLHDIK | CedarV_G_CT_8 |
| 82 | MNESLHDIK | CedarV_G_CT_9 |
| 83 | MLNESLHDIK | CedarV_G_CT_10 |
| 84 | MLLNESLHDIK | CedarV_G_CT_11 |
| 85 | MNLLNESLHDIK | CedarV_G_CT_12 |
| 86 | MSNLLNESLHDIK | CedarV_G_CT_13 |
| 87 | MVSNLLNESLHDIK | CedarV_G_CT_14 |
| 88 | MNVSNLLNESLHDIK | CedarV_G_CT_15 |
| 89 | MYNVSNLLNESLHDIK | CedarV_G_CT_16 |
| 90 | MNYNVSNLLNESLHDIK | CedarV_G_CT_17 |
| 91 | MKNYNVSNLLNESLHDIK | CedarV_G_CT_18 |
| 92 | MNKNYNVSNLLNESLHDIK | CedarV_G_CT_19 |
| 93 | MKNKNYNVSNLLNESLHDIK | CedarV_G_CT_20 |
| 94 | MVKNKNYNVSNLLNESLHDIK | CedarV_G_CT_21 |
| 95 | MYVKNKNYNVSNLLNESLHDIK | CedarV_G_CT_22 |
| 96 | MYYVKNKNYNVSNLLNESLHDIK | CedarV_G_CT_23 |
| 97 | MSYYVKNKNYNVSNLLNESLHDIK | CedarV_G_CT_24 |
| 98 | MQKDLNKSYYVKNKNYNVSNLLNESLHDIK | CedarV_G_CT_30 |
| 99 | MLSQLQKNYLDNSNQQGDKMNNPDKKLSVNFNPLEL | CedarV_G_CT_68_FL |
| | DKGQKDLNKSYYVKNKNYNVSNLLNESLHDIK | |
| 100 | MQGGRR | CDV-H_CT_06 |
| 101 | MEQGGRR | CDV-H_CT_07 |
| 102 | MEEQGGRR | CDV-H_CT_08 |
| 103 | MTEEQGGRR | CDV-H_CT_09 |
| 104 | MVTEEQGGRR | CDV-H_CT_10 |
| 105 | MLVTEEQGGRR | CDV-H_CT_11 |
| 106 | MSLVTEEQGGRR | CDV-H_CT_12 |
| 107 | MLSLVTEEQGGRR | CDV-H_CT_13 |
| 108 | MKLSLVTEEQGGRR | CDV-H_CT_14 |
| 109 | MSKLSLVTEEQGGRR | CDV-H_CT_15 |
| 110 | MSSKLSLVTEEQGGRR | CDV-H_CT_16 |
| 111 | MNSSKLSLVTEEQGGRR | CDV-H_CT_17 |
| 112 | MANSSKLSLVTEEQGGRR | CDV-H_CT_18 |
| 113 | MRANSSKLSLVTEEQGGRR | CDV-H_CT_19 |
| 114 | MARANSSKLSLVTEEQGGRR | CDV-H_CT_20 |
| 115 | MLSYQDKVGAFYKDNARANSSKLSLVTEEQGGRR | CDV-H_CT_34_FL |
| 116 | MRIIFR | HPIV2-HN_CT_06 |
| 117 | MIPKRTCRIIFR | HPIV2-HN_CT_12 |
| 118 | MEDYSNLSLKSIPKRTCRIIFR | HPIV2-HN_CT_22_FL |
| 119 | MLMIDR | MvH_CT_06 |
| 120 | MHLMIDR | MvH_CT_07 |
| 121 | MEHLMIDR | MvH_CT_08 |
| 122 | MREHLMIDR | MvH_CT_09 |
| 123 | MNREHLMIDR | MvH_CT_10 |
| 124 | MINREHLMIDR | MvH_CT_11 |
| 125 | MVINREHLMIDR | MvH_CT_12 |
| 126 | MIVINREHLMIDR | MvH_CT_13 |
| 127 | MRIVINREHLMIDR | MvH_CT_14 |
| 128 | MSRIVINREHLMIDR | MvH_CT_15 |
| 129 | MGSRIVINREHLMIDR | MvH_CT_16 |
| 130 | MKGSRIVINREHLMIDR | MvH_CT_17 |
| 131 | MPKGSRIVINREHLMIDR | MvH_CT_18 |
| 132 | MHPKGSRIVINREHLMIDR | MvH_CT_19 |
| 133 | MPHPKGSRIVINREHLMIDR | MvH_CT_20 |
| 134 | MSPQRDRINAFYKDNPHPKGSRIVINREHLMIDR | MvH_CT_34_FL |
| 135 | MRLVFR | NDV-HN_CT_06 |
| 136 | MWRLVFR | NDV-HN_CT_07 |
| 137 | MTWRLVFR | NDV-HN_CT_08 |
| 138 | MNTWRLVFR | NDV-HN_CT_09 |
| 139 | MKNTWRLVFR | NDV-HN_CT_10 |
| 140 | MAKNTWRLVFR | NDV-HN_CT_11 |
| 141 | MEAKNTWRLVFR | NDV-HN_CT_12 |
| 142 | MREAKNTWRLVFR | NDV-HN_CT_13 |
| 143 | MEREAKNTWRLVFR | NDV-HN_CT_14 |
| 144 | MDEREAKNTWRLVFR | NDV-HN_CT_15 |
| 145 | MNDEREAKNTWRLVFR | NDV-HN_CT_16 |
| 146 | MENDEREAKNTWRLVFR | NDV-HN_CT_17 |
| 147 | MLENDEREAKNTWRLVFR | NDV-HN_CT_18 |
| 148 | MALENDEREAKNTWRLVFR | NDV-HN_CT_19 |
| 149 | MVALENDEREAKNTWRLVFR | NDV-HN_CT_20 |
| 150 | MERGVSQVALENDEREAKNTWRLVFR | NDV-HN_CT_26_FL |
| 151 | MERSGK | SeV_CT_06 |
| 152 | MSERSGK | SeV_CT_07 |
| 153 | MDSERSGK | SeV_CT_08 |
| 154 | MSDSERSGK | SeV_CT_09 |
| 155 | MVSDSERSGK | SeV_CT_10 |
| 156 | MLVSDSERSGK | SeV_CT_11 |
| 157 | MKLVSDSERSGK | SeV_CT_12 |
| 158 | MTKLVSDSERSGK | SeV_CT_13 |
| 159 | MTTKLVSDSERSGK | SeV_CT_14 |
| 160 | MSTTKLVSDSERSGK | SeV_CT_15 |
| 161 | MGSTTKLVSDSERSGK | SeV_CT_16 |
| 162 | MGGSTTKLVSDSERSGK | SeV_CT_17 |
| 163 | MPGGSTTKLVSDSERSGK | SeV_CT_18 |
| 164 | MSPGGSTTKLVSDSERSGK | SeV_CT_19 |
| 165 | MTSPGGSTTKLVSDSERSGK | SeV_CT_20 |
| 166 | MDGDRSKRDSYWSTSPGGSTTKLVSDSERSGK | SeV_CT_32_FL |
| 167 | MDQAEEDTRLVQYQQTLVMAHIINLKDNIFATLRN | BaEVwt_CT |
| 168 | MAHIINLKDNIFATLRNKKINEGLLDSK | BaEVRLess_CT |
| 169 | MAHIINLKDNIFATLRN | BaEVRLess_CT |
| 170 | MKRFRSMEIDNYIRKNNSGQYRYR | Cocal_CT |
| 171 | MKRFRSMEIDNYIRKNNSGQYRYRKKINEGLLDSK | Cocal_CT |
| 172 | MFVFKC | EboV-GP_CT |
| 173 | MFVFKCKKINEGLLDSK | Ebo V-GP_CT |
| 174 | MLNGENELAQYKQRLVLIKVASIRDNIFQVLKKKINE | GaLV_CT |
| | GLLDSK | |
| 175 | MLNGENELAQYKQRLVLIKVASIRDNIFQVLK | GaLV_CT |
| 176 | MLIKVASIRDNIFQVLKKKINEGLLDSK | GaLV-RLess_CT |
| 177 | MLIKVASIRDNIFQVLK | GaLV-RLess_CT |
| 178 | MRRKWVTKVGPVKFAGCSCIGKNTLRHPKPCSGGKIH | LCMV_GP_CT |
| | RHT | |
| 179 | MPEYEIPKLQHYQQTLVLAQVVSIRDKVFQVLRKKIN | MLV-A_CT |
| | EGLLDSK | |
| 180 | MPEYEIPKLQHYQQTLVLAQVVSIRDKVFQVLR | MLV-A_CT |
| 181 | MLAQVVSIRDKVFQVLRKKINEGLLDSK | MLV-A-RLess_CT |
| 182 | MLAQVVSIRDKVFQVLR | MLV-A-RLess_CT |
| 183 | MKGLRNMEIDTYIQRKKTHKLKICLHIGVR | VSV-G_CT |
| 184 | MKGLRNMEIDTYIQRKKTHKLKICLHIGVRKKINEGLL | VSV-G_CT |
| | DSK | |
| 185 | MLLIRELGQRIRRPIHRIARYAAQLVEIVRDTGEAVAIA | HIV-1_gp41_MLLIR-LLP1- |
| | TANLLNVASNKLEQSWYQLLNWWYKLAEWGRRGLL | LLP3-LLP2-gp41-CT-N |
| | EVIRTVILLLDRLRHYSFLCLSRLDDWILALSGNVLRIS | |
| | RDRDREGGEEEIGEPRDPGRPIPLHTQFSLPSYGQRVR | |
| | N | |
| 186 | MLLIRELGQRIRRPIHRIARYAAQLVEIVRDTGEAVAIA | HIV-1_gp41_MLLIR-LLP1- |
| | TANLLNVASNKLEQSWYQLLNWWYKLAEWGRRGLL | LLP3-LLP2-gp41-CT-N |
| | EVIRTVILLLDRLRHYSFLCLSRLDDWILALSGNVLRIS | |
| | RDRDREGGEEEIGEPRDPGRPIPLHTQFSLPSYGQRVR | |
| | NKKINEGLLDSK | |
| 187 | MAVAIATANLLNVASNKLEQSWYQLLNWWYKLAEW | HIV-1_gp41_LLP3-LLP2- |
| | GRRGLLEVIRTVILLLDRLRHYSFLCLSRLDDWILALS | gp41-CT-N |
| | GNVLRISRDRDREGGEEEIGEPRDPGRPIPLHTQFSLPS | |
| | YGQRVRN | |
| 188 | MLLEVIRTVILLLDRLRHYSFLCLSRLDDWILALSGNV | HIV-1_gp41_LLP2-gp41- |
| | LRISRDRDREGGEEEIGEPRDPGRPIPLHTQFSLPSYGQ | CT-N |
| | RVRN | |
| 189 | MSGNVLRISRDRDREGGEEEIGEPRDPGRPIPLHTQFSL | HIV-1_gp41_gp41-CT-N |
| | PSYGQRVRN | |
| 190 | MLLIRELGQRIRRPIHRIARYAAQLVEIVRDTGEAVAIA | HIV-1_gp41_MLLIR-LLP1- |
| | TANLLNVASNKLEQSWYQLLNWWYKLAEWGRRGLL | LLP3-LLP2 |
| | EVIRTVILLLDRLRHYSFLCLSRLDDWILAL | |
| 191 | MELGQRIRRPIHRIARYAAQLVEIVRDTGEAVAIATAN | HIV-1_gp41_LLP1-LLP3- |
| | LLNVASNKLEQSWYQLLNWWYKLAEWGRRGLLEVI | LLP2 |
| | RTVILLLDRLRHYSFLCLSRLDDWILAL | |
| 192 | MAVAIATANLLNVASNKLEQSWYQLLNWWYKLAEW | HIV-1_gp41_LLP3-LLP2 |
| | GRRGLLEVIRTVILLLDRLRHYSFLCLSRLDDWILAL | |
| 193 | MLLEVIRTVILLLDRLRHYSFLCLSRLDDWILAL | HIV-1_gp41_LLP2 |
| 194 | MLLIRELGQRIRRPIHRIARYAAQLVEIVRDTGEAVAIA | HIV-1_gp41_MLLIR-LLP1- |
| | TANLLNVASNKLEQSWYQLLNWWYKLAEW | LLP3 |
| 195 | MELGQRIRRPIHRIARYAAQLVEIVRDTGEAVAIATAN | HIV-1_gp41_LLP1-LLP3 |
| | LLNVASNKLEQSWYQLLNWWYKLAEW | |
| 196 | MAVAIATANLLNVASNKLEQSWYQLLNWWYKLAEW | HIV-1_gp41_LLP3 |
| 197 | MLLIRELGQRIRRPIHRIARYAAQLVEIVR | HIV-1_gp41_MLLIR-LLP1 |
| 198 | MELGQRIRRPIHRIARYAAQLVEIVR | HIV-1_gp41_LLP1 |
| 199 | MLLIR | HIV-1_gp41_MLLIR |
| 200 | MAVEGGMKCVK | CD63_CT_1to11 |
| 201 | MCGACKENYC | CD63_CT_73to81 |
| 202 | MVEYGSRISKVLCC | CD63_CT_225to238 |
| 203 | MAVEGGMKCVKKKINEGLLDSK | CD63_CT_1to11 |
| 204 | MCGACKENYCKKINEGLLDSK | CD63_CT_73to81 |
| 205 | MVEYGSRISKVLCCKKINEGLLDSK | CD63_CT_225to238 |
| 206 | MKGEYKPNVVTTVASKYIPNEGTDWKANMKEKEFK | CD29_CT |
| | AFERRDHIIMLLK | |
| 207 | MKGEYKPNVVTTVASKYIPNEGTDWKANMKEKEFK | CD29_CT |
| | AFERRDHIIMLLKKKINEGLLDSK | |
| 208 | MVINREHLMIDRILSAFNTVIALLGSIVIIVMNIMIIQNY | MvH_CT_12 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 209 | MGSRIVINREHLMIDRILSAFNTVIALLGSIVIIVMNIMII | MvH_CT_16 |
| | QNYTRSTDNQAVIKDALQGIQQQIKGLADKIGTEIGPK | |
| | VSLIDTSSTITIPANIGLLGSKISQSTASINENVNEKCKFT | |
| | LPPLKIHECNISCPNPLPFREYRPQTEGVSNLVGLPNNI | |
| | CLQKTSNQILKPKLISYTLPVVGQSGTCITDPLLAMDE | |
| | GYFAYSHLERIGSCSRGVSKQRIIGVGEVLDRGDEVPS | |
| | LFMTNVWTPPNPNTVYHCSAVYNNEFYYVLCAVSTV | |
| | GDPILNSTYWSGSLMMTRLAVKPKSNGGGYNQHQLA | |
| | LRSIEKGRYDKVMPYGPSGIKQGDTLYFPAVGFLVRT | |
| | EFKYNDSNCPITKCQYSKPENCRLSMGIRPNSHYILRS | |
| | GLLKYNLSDGENPKVVFIEISDQRLSIGSPSKIYDSLGQ | |
| | PVFYQASFSWDTMIKFGDVLTVNPLVVNWRNNTVISR | |
| | PGQSQCPRFNTCPAICAEGVYNDAFLIDRINWISAGVF | |
| | LDSNATAANPVFTVFKDNEILYRAQLASEDTNAQKTIT | |
| | NCFLLKNKIWCISLVEIYDTGDNVIRPKLFAVKIPEQCT | |
| 210 | MPHPKGSRIVINREHLMIDRILSAFNTVIALLGSIVIIVM | MvH_CT_20 |
| | NIMIIQNYTRSTDNQAVIKDALQGIQQQIKGLADKIGT | |
| | EIGPKVSLIDTSSTITIPANIGLLGSKISQSTASINENVNE | |
| | KCKFTLPPLKIHECNISCPNPLPFREYRPQTEGVSNLVG | |
| | LPNNICLQKTSNQILKPKLISYTLPVVGQSGTCITDPLL | |
| | AMDEGYFAYSHLERIGSCSRGVSKQRIIGVGEVLDRG | |
| | DEVPSLFMTNVWTPPNPNTVYHCSAVYNNEFYYVLC | |
| | AVSTVGDPILNSTYWSGSLMMTRLAVKPKSNGGGYN | |
| | QHQLALRSIEKGRYDKVMPYGPSGIKQGDTLYFPAVG | |
| | FLVRTEFKYNDSNCPITKCQYSKPENCRLSMGIRPNSH | |
| | YILRSGLLKYNLSDGENPKVVFIEISDQRLSIGSPSKIYD | |
| | SLGQPVFYQASFSWDTMIKFGDVLTVNPLVVNWRNN | |
| | TVISRPGQSQCPRFNTCPAICAEGVYNDAFLIDRINWIS | |
| | AGVFLDSNATAANPVFTVFKDNEILYRAQLASEDTNA | |
| | QKTITNCFLLKNKIWCISLVEIYDTGDNVIRPKLFAVKI | |
| | PEQCT | |
| 211 | MYYGTMDIKKINEGLLDSKILSAFNTVIALLGSIVIIVM | NivG_CT_19 |
| | NIMIIQNYTRSTDNQAVIKDALQGIQQQIKGLADKIGT | |
| | EIGPKVSLIDTSSTITIPANIGLLGSKISQSTASINENVNE | |
| | KCKFTLPPLKIHECNISCPNPLPFREYRPQTEGVSNLVG | |
| | LPNNICLQKTSNQILKPKLISYTLPVVGQSGTCITDPLL | |
| | AMDEGYFAYSHLERIGSCSRGVSKQRIIGVGEVLDRG | |
| | DEVPSLFMTNVWTPPNPNTVYHCSAVYNNEFYYVLC | |
| | AVSTVGDPILNSTYWSGSLMMTRLAVKPKSNGGGYN | |
| | QHQLALRSIEKGRYDKVMPYGPSGIKQGDTLYFPAVG | |
| | FLVRTEFKYNDSNCPITKCQYSKPENCRLSMGIRPNSH | |
| | YILRSGLLKYNLSDGENPKVVFIEISDQRLSIGSPSKIYD | |
| | SLGQPVFYQASFSWDTMIKFGDVLTVNPLVVNWRNN | |
| | TVISRPGQSQCPRFNTCPAICAEGVYNDAFLIDRINWIS | |
| | AGVFLDSNATAANPVFTVFKDNEILYRAQLASEDTNA | |
| | QKTITNCFLLKNKIWCISLVEIYDTGDNVIRPKLFAVKI | |
| | PEQCT | |
| 212 | MKNQNDHYILSAFNTVIALLGSIVIIVMNIMIIQNYTRS | BatPV_G_CT_8 |
| | TDNQAVIKDALQGIQQQIKGLADKIGTEIGPKVSLIDTS | |
| | STITIPANIGLLGSKISQSTASINENVNEKCKFTLPPLKIH | |
| | ECNISCPNPLPFREYRPQTEGVSNLVGLPNNICLQKTSN | |
| | QILKPKLISYTLPVVGQSGTCITDPLLAMDEGYFAYSH | |
| | LERIGSCSRGVSKQRIIGVGEVLDRGDEVPSLFMTNVW | |
| | TPPNPNTVYHCSAVYNNEFYYVLCAVSTVGDPILNST | |
| | YWSGSLMMTRLAVKPKSNGGGYNQHQLALRSIEKGR | |
| | YDKVMPYGPSGIKQGDTLYFPAVGFLVRTEFKYNDSN | |
| | CPITKCQYSKPENCRLSMGIRPNSHYILRSGLLKYNLS | |
| | DGENPKVVFIEISDQRLSIGSPSKIYDSLGQPVFYQASFS | |
| | WDTMIKFGDVLTVNPLVVNWRNNTVISRPGQSQCPRF | |
| | NTCPAICAEGVYNDAFLIDRINWISAGVFLDSNATAAN | |
| | PVFTVFKDNEILYRAQLASEDTNAQKTITNCFLLKNKI | |
| | WCISLVEIYDTGDNVIRPKLFAVKIPEQCT | |
| 213 | MDSERSGKILSAFNTVIALLGSIVIIVMNIMIIQNYTRST | SeV_CT_08 |
| | DNQAVIKDALQGIQQQIKGLADKIGTEIGPKVSLIDTSS | |
| | TITIPANIGLLGSKISQSTASINENVNEKCKFTLPPLKIHE | |
| | CNISCPNPLPFREYRPQTEGVSNLVGLPNNICLQKTSN | |
| | QILKPKLISYTLPVVGQSGTCITDPLLAMDEGYFAYSH | |
| | LERIGSCSRGVSKQRIIGVGEVLDRGDEVPSLFMTNVW | |
| | TPPNPNTVYHCSAVYNNEFYYVLCAVSTVGDPILNST | |
| | YWSGSLMMTRLAVKPKSNGGGYNQHQLALRSIEKGR | |
| | YDKVMPYGPSGIKQGDTLYFPAVGFLVRTEFKYNDSN | |
| | CPITKCQYSKPENCRLSMGIRPNSHYILRSGLLKYNLS | |
| | DGENPKVVFIEISDQRLSIGSPSKIYDSLGQPVFYQASFS | |
| | WDTMIKFGDVLTVNPLVVNWRNNTVISRPGQSQCPRF | |
| | NTCPAICAEGVYNDAFLIDRINWISAGVFLDSNATAAN | |
| | PVFTVFKDNEILYRAQLASEDTNAQKTITNCFLLKNKI | |
| | WCISLVEIYDTGDNVIRPKLFAVKIPEQCT | |
| 214 | MLAQVVSIRDKVFQVLRILSAFNTVIALLGSIVIIVMNI | MLV-A-RLess_CT |
| | MIIQNYTRSTDNQAVIKDALQGIQQQIKGLADKIGTEI | |
| | GPKVSLIDTSSTITIPANIGLLGSKISQSTASINENVNEK | |
| | CKFTLPPLKIHECNISCPNPLPFREYRPQTEGVSNLVGL | |
| | PNNICLQKTSNQILKPKLISYTLPVVGQSGTCITDPLLA | |
| | MDEGYFAYSHLERIGSCSRGVSKQRIIGVGEVLDRGDE | |
| | VPSLFMTNVWTPPNPNTVYHCSAVYNNEFYYVLCAV | |
| | STVGDPILNSTYWSGSLMMTRLAVKPKSNGGGYNQH | |
| | QLALRSIEKGRYDKVMPYGPSGIKQGDTLYFPAVGFL | |
| | VRTEFKYNDSNCPITKCQYSKPENCRLSMGIRPNSHYI | |
| | LRSGLLKYNLSDGENPKVVFIEISDQRLSIGSPSKIYDSL | |
| | GQPVFYQASFSWDTMIKFGDVLTVNPLVVNWRNNTV | |
| | ISRPGQSQCPRFNTCPAICAEGVYNDAFLIDRINWISAG | |
| | VFLDSNATAANPVFTVFKDNEILYRAQLASEDTNAQK | |
| | TITNCFLLKNKIWCISLVEIYDTGDNVIRPKLFAVKIPE | |
| | QCT | |
| 215 | MLLIRILSAFNTVIALLGSIVIIVMNIMIIQNYTRSTDNQ | HIV-1_gp41_MLLIR |
| | AVIKDALQGIQQQIKGLADKIGTEIGPKVSLIDTSSTITI | |
| | PANIGLLGSKISQSTASINENVNEKCKFTLPPLKIHECNI | |
| | SCPNPLPFREYRPQTEGVSNLVGLPNNICLQKTSNQILK | |
| | PKLISYTLPVVGQSGTCITDPLLAMDEGYFAYSHLERI | |
| | GSCSRGVSKQRIIGVGEVLDRGDEVPSLFMTNVWTPP | |
| | NPNTVYHCSAVYNNEFYYVLCAVSTVGDPILNSTYWS | |
| | GSLMMTRLAVKPKSNGGGYNQHQLALRSIEKGRYDK | |
| | VMPYGPSGIKQGDTLYFPAVGFLVRTEFKYNDSNCPIT | |
| | KCQYSKPENCRLSMGIRPNSHYILRSGLLKYNLSDGEN | |
| | PKVVFIEISDQRLSIGSPSKIYDSLGQPVFYQASFSWDT | |
| | MIKFGDVLTVNPLVVNWRNNTVISRPGQSQCPRFNTC | |
| | PAICAEGVYNDAFLIDRINWISAGVFLDSNATAANPVF | |
| | TVFKDNEILYRAQLASEDTNAQKTITNCFLLKNKIWCI | |
| | SLVEIYDTGDNVIRPKLFAVKIPEQCT | |
| 216 | MAVEGGMKCVKILSAFNTVIALLGSIVIIVMNIMIIQNY | CD63_CT_1to11 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 217 | MCGACKENYCILSAFNTVIALLGSIVIIVMNIMIIQNYT | CD63_CT_73to81 |
| | RSTDNQAVIKDALQGIQQQIKGLADKIGTEIGPKVSLID | |
| | TSSTITIPANIGLLGSKISQSTASINENVNEKCKFTLPPL | |
| | KIHECNISCPNPLPFREYRPQTEGVSNLVGLPNNICLQK | |
| | TSNQILKPKLISYTLPVVGQSGTCITDPLLAMDEGYFA | |
| | YSHLERIGSCSRGVSKQRIIGVGEVLDRGDEVPSLFMT | |
| | NVWTPPNPNTVYHCSAVYNNEFYYVLCAVSTVGDPIL | |
| | NSTYWSGSLMMTRLAVKPKSNGGGYNQHQLALRSIE | |
| | KGRYDKVMPYGPSGIKQGDTLYFPAVGFLVRTEFKYN | |
| | DSNCPITKCQYSKPENCRLSMGIRPNSHYILRSGLLKY | |
| | NLSDGENPKVVFIEISDQRLSIGSPSKIYDSLGQPVFYQ | |
| | ASFSWDTMIKFGDVLTVNPLVVNWRNNTVISRPGQSQ | |
| | CPRFNTCPAICAEGVYNDAFLIDRINWISAGVFLDSNA | |
| | TAANPVFTVFKDNEILYRAQLASEDTNAQKTITNCFLL | |
| | KNKIWCISLVEIYDTGDNVIRPKLFAVKIPEQCT | |
| 218 | MKKINDGLLDSKILSAFNTVIALLGSIVIIVMNIMIIQNY | HevG_CT_12 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 219 | MPEYEIPKLQHYQQTLVLAQVVSIRDKVFQVLRILSAF | MLV-A_CT |
| | NTVIALLGSIVIIVMNIMIIQNYTRSTDNQAVIKDALQGI | |
| | QQQIKGLADKIGTEIGPKVSLIDTSSTITIPANIGLLGSKI | |
| | SQSTASINENVNEKCKFTLPPLKIHECNISCPNPLPFREY | |
| | RPQTEGVSNLVGLPNNICLQKTSNQILKPKLISYTLPVV | |
| | GQSGTCITDPLLAMDEGYFAYSHLERIGSCSRGVSKQR | |
| | IIGVGEVLDRGDEVPSLFMTNVWTPPNPNTVYHCSAV | |
| | YNNEFYYVLCAVSTVGDPILNSTYWSGSLMMTRLAV | |
| | KPKSNGGGYNQHQLALRSIEKGRYDKVMPYGPSGIKQ | |
| | GDTLYFPAVGFLVRTEFKYNDSNCPITKCQYSKPENCR | |
| | LSMGIRPNSHYILRSGLLKYNLSDGENPKVVFIEISDQR | |
| | LSIGSPSKIYDSLGQPVFYQASFSWDTMIKFGDVLTVN | |
| | PLVVNWRNNTVISRPGQSQCPRFNTCPAICAEGVYND | |
| | AFLIDRINWISAGVFLDSNATAANPVFTVFKDNEILYR | |
| | AQLASEDTNAQKTITNCFLLKNKIWCISLVEIYDTGDN | |
| | VIRPKLFAVKIPEQCT | |
| 220 | MLLDSKILSAFNTVIALLGSIVIIVMNIMIIQNYTRSTDN | NivG_CT_06 |
| | QAVIKDALQGIQQQIKGLADKIGTEIGPKVSLIDTSSTIT | |
| | IPANIGLLGSKISQSTASINENVNEKCKFTLPPLKIHECN | |
| | ISCPNPLPFREYRPQTEGVSNLVGLPNNICLQKTSNQIL | |
| | KPKLISYTLPVVGQSGTCITDPLLAMDEGYFAYSHLER | |
| | IGSCSRGVSKQRIIGVGEVLDRGDEVPSLFMTNVWTPP | |
| | NPNTVYHCSAVYNNEFYYVLCAVSTVGDPILNSTYWS | |
| | GSLMMTRLAVKPKSNGGGYNQHQLALRSIEKGRYDK | |
| | VMPYGPSGIKQGDTLYFPAVGFLVRTEFKYNDSNCPIT | |
| | KCQYSKPENCRLSMGIRPNSHYILRSGLLKYNLSDGEN | |
| | PKVVFIEISDQRLSIGSPSKIYDSLGQPVFYQASFSWDT | |
| | MIKFGDVLTVNPLVVNWRNNTVISRPGQSQCPRFNTC | |
| | PAICAEGVYNDAFLIDRINWISAGVFLDSNATAANPVF | |
| | TVFKDNEILYRAQLASEDTNAQKTITNCFLLKNKIWCI | |
| | SLVEIYDTGDNVIRPKLFAVKIPEQCT | |
| 221 | MIKKINEGLLDSKILSAFNTVIALLGSIVIIVMNIMIIQN | NivG_CT_13 |
| | YTRSTDNQAVIKDALQGIQQQIKGLADKIGTEIGPKVS | |
| | LIDTSSTITIPANIGLLGSKISQSTASINENVNEKCKFTLP | |
| | PLKIHECNISCPNPLPFREYRPQTEGVSNLVGLPNNICL | |
| | QKTSNQILKPKLISYTLPVVGQSGTCITDPLLAMDEGY | |
| | FAYSHLERIGSCSRGVSKQRIIGVGEVLDRGDEVPSLF | |
| | MTNVWTPPNPNTVYHCSAVYNNEFYYVLCAVSTVGD | |
| | PILNSTYWSGSLMMTRLAVKPKSNGGGYNQHQLALR | |
| | SIEKGRYDKVMPYGPSGIKQGDTLYFPAVGFLVRTEF | |
| | KYNDSNCPITKCQYSKPENCRLSMGIRPNSHYILRSGL | |
| | LKYNLSDGENPKVVFIEISDQRLSIGSPSKIYDSLGQPV | |
| | FYQASFSWDTMIKFGDVLTVNPLVVNWRNNTVISRPG | |
| | QSQCPRFNTCPAICAEGVYNDAFLIDRINWISAGVFLD | |
| | SNATAANPVFTVFKDNEILYRAQLASEDTNAQKTITN | |
| | CFLLKNKIWCISLVEIYDTGDNVIRPKLFAVKIPEQCT | |
| 222 | MWKKQKNQNDHYILSAFNTVIALLGSIVIIVMNIMIIQ | BatPV_G_CT_12 |
| | NYTRSTDNQAVIKDALQGIQQQIKGLADKIGTEIGPKV | |
| | SLIDTSSTITIPANIGLLGSKISQSTASINENVNEKCKFTL | |
| | PPLKIHECNISCPNPLPFREYRPQTEGVSNLVGLPNNIC | |
| | LQKTSNQILKPKLISYTLPVVGQSGTCITDPLLAMDEG | |
| | YFAYSHLERIGSCSRGVSKQRIIGVGEVLDRGDEVPSL | |
| | FMTNVWTPPNPNTVYHCSAVYNNEFYYVLCAVSTVG | |
| | DPILNSTYWSGSLMMTRLAVKPKSNGGGYNQHQLAL | |
| | RSIEKGRYDKVMPYGPSGIKQGDTLYFPAVGFLVRTEF | |
| | KYNDSNCPITKCQYSKPENCRLSMGIRPNSHYILRSGL | |
| | LKYNLSDGENPKVVFIEISDQRLSIGSPSKIYDSLGQPV | |
| | FYQASFSWDTMIKFGDVLTVNPLVVNWRNNTVISRPG | |
| | QSQCPRFNTCPAICAEGVYNDAFLIDRINWISAGVFLD | |
| | SNATAANPVFTVFKDNEILYRAQLASEDTNAQKTITN | |
| | CFLLKNKIWCISLVEIYDTGDNVIRPKLFAVKIPEQCT | |
| 223 | MRIIFRILSAFNTVIALLGSIVIIVMNIMIIQNYTRSTDNQ | HPIV2-HN_CT_06 |
| | AVIKDALQGIQQQIKGLADKIGTEIGPKVSLIDTSSTITI | |
| | PANIGLLGSKISQSTASINENVNEKCKFTLPPLKIHECNI | |
| | SCPNPLPFREYRPQTEGVSNLVGLPNNICLQKTSNQILK | |
| | PKLISYTLPVVGQSGTCITDPLLAMDEGYFAYSHLERI | |
| | GSCSRGVSKQRIIGVGEVLDRGDEVPSLFMTNVWTPP | |
| | NPNTVYHCSAVYNNEFYYVLCAVSTVGDPILNSTYWS | |
| | GSLMMTRLAVKPKSNGGGYNQHQLALRSIEKGRYDK | |
| | VMPYGPSGIKQGDTLYFPAVGFLVRTEFKYNDSNCPIT | |
| | KCQYSKPENCRLSMGIRPNSHYILRSGLLKYNLSDGEN | |
| | PKVVFIEISDQRLSIGSPSKIYDSLGQPVFYQASFSWDT | |
| | MIKFGDVLTVNPLVVNWRNNTVISRPGQSQCPRFNTC | |
| | PAICAEGVYNDAFLIDRINWISAGVFLDSNATAANPVF | |
| | TVFKDNEILYRAQLASEDTNAQKTITNCFLLKNKIWCI | |
| | SLVEIYDTGDNVIRPKLFAVKIPEQCT | |
| 224 | MIPKRTCRIIFRILSAFNTVIALLGSIVIIVMNIMIIQNYT | HPIV2-HN_CT_12 |
| | RSTDNQAVIKDALQGIQQQIKGLADKIGTEIGPKVSLID | |
| | TSSTITIPANIGLLGSKISQSTASINENVNEKCKFTLPPL | |
| | KIHECNISCPNPLPFREYRPQTEGVSNLVGLPNNICLQK | |
| | TSNQILKPKLISYTLPVVGQSGTCITDPLLAMDEGYFA | |
| | YSHLERIGSCSRGVSKQRIIGVGEVLDRGDEVPSLFMT | |
| | NVWTPPNPNTVYHCSAVYNNEFYYVLCAVSTVGDPIL | |
| | NSTYWSGSLMMTRLAVKPKSNGGGYNQHQLALRSIE | |
| | KGRYDKVMPYGPSGIKQGDTLYFPAVGFLVRTEFKYN | |
| | DSNCPITKCQYSKPENCRLSMGIRPNSHYILRSGLLKY | |
| | NLSDGENPKVVFIEISDQRLSIGSPSKIYDSLGQPVFYQ | |
| | ASFSWDTMIKFGDVLTVNPLVVNWRNNTVISRPGQSQ | |
| | CPRFNTCPAICAEGVYNDAFLIDRINWISAGVFLDSNA | |
| | TAANPVFTVFKDNEILYRAQLASEDTNAQKTITNCFLL | |
| | KNKIWCISLVEIYDTGDNVIRPKLFAVKIPEQCT | |
| 225 | MKLVSDSERSGKILSAFNTVIALLGSIVIIVMNIMIIQNY | CD63_CT_1to11 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 226 | MVVILDKRCY CNLLILILMI SECSVGILHY | Truncated NiV fusion |
| | EKLSKIGLVK GVTRKYKIKS NPLTKDIVIK | glycoprotein (FcDelta22) at |
| | MIPNVSNMSQ CTGSVMENYK TRLNGILTPI | cytoplasmic tail |
| | KGALEIYKNN THDLVGDVRL AGVIMAGVAI | (with signal sequence) |
| | GIATAAQITA GVALYEAMKN ADNINKLKSS | |
| | IESTNEAVVK LQETAEKTVY VLTALQDYI | |
| | TNLVPTIDKI SCKQTELSLD LALSKYLSDL | |
| | LFVFGPNLQD PVSNSMTIQA ISQAFGGNYE | |
| | TLLRTLGYAT EDFDDLLESD SITGQIIYVD | |
| | LSSYYIIVRV YFPILTEIQQ AYIQELLPVS | |
| | FNNDNSEWIS IVPNFILVRN TLISNIEIGF CLITKRSVIC | |
| | NQDYATPMTN NMRECLTGST EKCPRELVVS | |
| | SHVPRFALSN GVLFANCISV TCQCQTTGRA | |
| | ISQSGEQTLL MIDNTTCPTA VLGNVIISLG | |
| | KYLGSVNYNS EGIAIGPPVF TDKVDISSQI | |
| | SSMNQSLQQS KDYIKEAQRL LDTVNPSLIS | |
| | MLSMIILYVL SIASLCIGLI TFISFIIVEK KRNT | |
| 227 | ILHY EKLSKIGLVKG VTRKYKIKSN PLTKDIVIK | Truncated mature NiV |
| | MIPNVSNMSQ CTGSVMENYK TRLNGILTPI | fusion glycoprotein |
| | KGALEIYKNN THDLVGDVRL AGVIMAGVAI | (FcDelta22) at cytoplasmic |
| | GIATAAQITA GVALYEAMKN ADNINKLKSS | tail |
| | IESTNEAVVK LQETAEKTVY VLTALQDYIN | |
| | TNLVPTIDKI SCKQTELSLD LALSKYLSDL | |
| | LFVFGPNLQD PVSNSMTIQA ISQAFGGNYE | |
| | TLLRTLGYAT EDFDDLLESD SITGQIIYVD | |
| | LSSYYIIVRV YFPILTEIQQ AYIQELLPVS | |
| | FNNDNSEWIS IVPNFILVRN TLISNIEIGF CLITKRSVIC | |
| | NQDYATPMTN NMRECLTGST EKCPRELVVS | |
| | SHVPRFALSN GVLFANCISV TCQCQTTGRA | |
| | ISQSGEQTLL MIDNTTCPTA VLGNVIISLG | |
| | KYLGSVNYNS EGIAIGPPVF TDKVDISSQI | |
| | SSMNQSLQQS KDYIKEAQRL LDTVNPSLIS | |
| | MLSMIILYVL SIASLCIGLI TFISFIIVEK KRNT | |
| 228 | MKKINEGLLDSKILSA FNTVIALLGS IVIIVMNIMI | NiVG protein attachment |
| | IQNYTRSTDN QAVIKDALQG IQQQIKGLAD | glycoprotein |
| | KIGTEIGPKV SLIDTSSTIT IPANIGLLGS KISQSTASIN | Truncated and mutated |
| | ENVNEKCKFT LPPLKIHECN ISCPNPLPFR | (E501A, W504A, Q530A, |
| | EYRPQTEGVS NLVGLPNNIC LQKTSNQILK | E533A) NiV G protein (Gc |
| | PKLISYTLPV VGQSGTCITD PLLAMDEGYF | Δ 34) |
| | AYSHLERIGS CSRGVSKQRI IGVGEVLDRG | |
| | DEVPSLFMTN VWTPPNPNTV YHCSAVYNNE | |
| | FYYVLCAVST VGDPILNSTY WSGSLMMTRL | |
| | AVKPKSNGGG YNQHQLALRS IEKGRYDKVM | |
| | PYGPSGIKQG DTLYFPAVGF LVRTEFKYND | |
| | SNCPITKCQY SKPENCRLSM GIRPNSHYIL | |
| | RSGLLKYNLS DGENPKVVFI EISDQRLSIG | |
| | SPSKIYDSLG QPVFYQASFS WDTMIKFGDV | |
| | LTVNPLVVNW RNNTVISRPG QSQCPRFNTC | |
| | PAICAEGVYN DAFLIDRINW ISAGVFLDSN | |
| | ATAANPVFTV FKDNEILYRA QLASEDTNAQ | |
| | KTITNCFLLK NKIWCISLVE IYDTGDNVIR | |
| | PKLFAVKIPE QCT | |
| 229 | NRLVQFVKDRISVVQALVLTQQYHQLKPIEYEP | Moloney murine leukemia |
| | | virus cytoplasmic tail |
| | | (632-665 of UniProt |
| | | P03385; R-peptide 650-665) |
| 230 | GGGGS | peptide linker |
| 231 | GGGGGS | peptide linker |
| 232 | (GGGGS)n | peptide linker, wherein n is 1 |
| | | to 10 |
| 233 | (GGGGGS)n | peptide linker, wherein n is 1 |
| | | to 6 |
| 234 | MATQEVRLKCLLCGIIVLVLSLEGLGILHYEKLSKIGL | Hendra F |
| | VKGITRKYKIKSNPLTKDIVIKMIPNVSNVSKCTGTVM | |
| | ENYKSRLTGILSPIKGAIELYNNNTHDLVGDVKLAGV | |
| | VMAGIAIGIATAAQITAGVALYEAMKNADNINKLKSSI | |
| | ESTNEAVVKLQETAEKTVYVLTALQDYINTNLVPTID | |
| | QISCKQTELALDLALSKYLSDLLFVFGPNLQDPVSNSM | |
| | TIQAISQAFGGNYETLLRTLGYATEDFDDLLESDSIAG | |
| | QIVYVDLSSYYIIVRVYFPILTEIQQAYVQELLPVSENN | |
| | DNSEWISIVPNFVLIRNTLISNIEVKYCLITKKSVICNQD | |
| | YATPMTASVRECLTGSTDKCPRELVVSSHVPRFALSG | |
| | GVLFANCISVTCQCQTTGRAISQSGEQTLLMIDNTTCT | |
| | TVVLGNIIISLGKYLGSINYNSESIAVGPPVYTDKVDISS | |
| | QISSMNQSLQQSKDYIKEAQKILDTVNPSLISMLSMIIL | |
| | YVLSIAALCIGLITFISFVIVEKKRGNYSRLDDRQVRPV | |
| | SNGDLYYIGT | |
| 235 | MVVILDKRCYCNLLILILMISECSVGILHYEKLSKIGLV | Nipah F0 |
| | KGVTRKYKIKSNPLTKDIVIKMIPNVSNMSQCTGSVM | |
| | ENYKTRLNGILTPIKGALEIYKNNTHDLVGDVRLAGVI | |
| | MAGVAIGIATAAQITAGVALYEAMKNADNINKLKSSI | |
| | ESTNEAVVKLQETAEKTVYVLTALQDYINTNLVPTID | |
| | KISCKQTELSLDLALSKYLSDLLFVFGPNLQDPVSNSM | |
| | TIQAISQAFGGNYETLLRTLGYATEDFDDLLESDSITGQ | |
| | IIYVDLSSYYIIVRVYFPILTEIQQAYIQELLPVSFNNDN | |
| | SEWISIVPNFILVRNTLISNIEIGFCLITKRSVICNQDYAT | |
| | PMTNNMRECLTGSTEKCPRELVVSSHVPRFALSNGVL | |
| | FANCISVTCQCQTTGRAISQSGEQTLLMIDNTTCPTAV | |
| | LGNVIISLGKYLGSVNYNSEGIAIGPPVFTDKVDISSQIS | |
| | SMNQSLQQSKDYIKEAQRLLDTVNPSLISMLSMIILYV | |
| | LSIASLCIGLITFISFIIVEKKRNTYSRLEDRRVRPTSSGD | |
| | LYYIGT | |
| 236 | MSNKRTTVLIIISYTLFYLNNAAIVGFDFDKLNKIGVV | CedV F |
| | QGRVLNYKIKGDPMTKDLVLKFIPNIVNITECVREPLS | |
| | RYNETVRRLLLPIHNMLGLYLNNTNAKMTGLMIAGVI | |
| | MGGIAIGIATAAQITAGFALYEAKKNTENIQKLTDSIM | |
| | KTQDSIDKLTDSVGTSILILNKLQTYINNQLVPNLELLS | |
| | CRQNKIEFDLMLTKYLVDLMTVIGPNINNPVNKDMTI | |
| | QSLSLLFDGNYDIMMSELGYTPQDFLDLIESKSITGQII | |
| | YVDMENLYVVIRTYLPTLIEVPDAQIYEFNKITMSSNG | |
| | GEYLSTIPNFILIRGNYMSNIDVATCYMTKASVICNQD | |
| | YSLPMSQNLRSCYQGETEYCPVEAVIASHSPRFALTNG | |
| | VIFANCINTICRCQDNGKTITQNINQFVSMIDNSTCNDV | |
| | MVDKFTIKVGKYMGRKDINNINIQIGPQIIIDKVDLSNE | |
| | INKMNQSLKDSIFYLREAKRILDSVNISLISPSVQLFLIII | |
| | SVLSFIILLIIIVYLYCKSKHSYKYNKFIDDPDYYNDYK | |
| | RERINGKASKSNNIYYVGD | |
| 237 | MALNKNMFSSLFLGYLLVYATTVQSSIHYDSLSKVGV | Mojiang F |
| | IKGLTYNYKIKGSPSTKLMVVKLIPNIDSVKNCTQKQY | |
| | DEYKNLVRKALEPVKMAIDTMLNNVKSGNNKYRFAG | |
| | AIMAGVALGVATAATVTAGIALHRSNENAQAIANMK | |
| | SAIQNTNEAVKQLQLANKQTLAVIDTIRGEINNNIIPVI | |
| | NQLSCDTIGLSVGIRLTQYYSEIITAFGPALQNPVNTRIT | |
| | IQAISSVFNGNFDELLKIMGYTSGDLYEILHSELIRGNII | |
| | DVDVDAGYIALEIEFPNLTLVPNAVVQELMPISYNIDG | |
| | DEWVTLVPRFVLTRTTLLSNIDTSRCTITDSSVICDNDY | |
| | ALPMSHELIGCLQGDTSKCAREKVVSSYVPKFALSDG | |
| | LVYANCLNTICRCMDTDTPISQSLGATVSLLDNKRCSV | |
| | YQVGDVLISVGSYLGDGEYNADNVELGPPIVIDKIDIG | |
| | NQLAGINQTLQEAEDYIEKSEEFLKGVNPSIITLGSMV | |
| | VLYIFMILIAIVSVIALVLSIKLTVKGNVVRQQFTYTQH | |
| | VPSMENINYVSH | |
| 238 | MKKKTDNPTISKRGHNHSRGIKSRALLRETDNYSNGLI | Bat PV F |
| | VENLVRNCHHPSKNNLNYTKTQKRDSTIPYRVEERKG | |
| | HYPKIKHLIDKSYKHIKRGKRRNGHNGNIITIILLLILIL | |
| | KTQMSEGAIHYETLSKIGLIKGITREYKVKGTPSSKDIV | |
| | IKLIPNVTGLNKCTNISMENYKEQLDKILIPINNIIELYA | |
| | NSTKSAPGNARFAGVIIAGVALGVAAAAQITAGIALHE | |
| | ARQNAERINLLKDSISATNNAVAELQEATGGIVNVITG | |
| | MQDYINTNLVPQIDKLQCSQIKTALDISLSQYYSEILTV | |
| | FGPNLQNPVTTSMSIQAISQSFGGNIDLLLNLLGYTAN | |
| | DLLDLLESKSITGQITYINLEHYFMVIRVYYPIMTTISN | |
| | AYVQELIKISFNVDGSEWVSLVPSYILIRNSYLSNIDISE | |
| | CLITKNSVICRHDFAMPMSYTLKECLTGDTEKCPREA | |
| | VVTSYVPRFAISGGVIYANCLSTTCQCYQTGKVIAQDG | |
| | SQTLMMIDNQTCSIVRIEEILISTGKYLGSQEYNTMHVS | |
| | VGNPVFTDKLDITSQISNINQSIEQSKFYLDKSKAILDKI | |
| | NLNLIGSVPISILFIIAILSLILSIITFVIVMIIVRRYNKYTP | |
| | LINSDPSSRRSTIQDVYIIPNPGEHSIRSAARSIDRDRD | |
| 239 | DIQMTQTTSSLSASLGDRVTISCRASQDISKYLNWYQQ | Anti-CD19 scFv (FMC63) |
| | KPDGTVKLLIYHTSRLHSGVPSRFSGSGSGTDYSLTISN | |
| | LEQEDIATYFCQQGNTLPYTFGGGTKLEITGSTSGSGK | |
| | PGSGEGSTKGEVKLQESGPGLVAPSQSLSVTCTVSGVS | |
| | LPDYGVSWIRQPPRKGLEWLGVIWGSETTYYNSALKS | |
| | RLTIIKDNSKSQVFLKMNSLQTDDTAIYYCAKHYYYG | |
| | GSYAMDYWGQGTSVTVSS | |
| 240 | DIQMTQTTSSLSASLGDRVTISCRASQDISKYLNWYQQ | Anti-CD19 scFv (FMC63) |
| | KPDGTVKLLIYHTSRLHSGVPSRFSGSGSGTDYSLTISN | |
| | LEQEDIATYFCQQGNTLPYTFGGGTKLEITGGGGSGG | |
| | GGSGGGGSEVKLQESGPGLVAPSQSLSVTCTVSGVSLP | |
| | DYGVSWIRQPPRKGLEWLGVIWGSETTYYNSALKSRL | |
| | TIIKDNSKSQVFLKMNSLQTDDTAIYYCAKHYYYGGS | |
| | YAMDYWGQGTSVTVSS | |
| 241 | ESKYGPPCPPCP | IgG4 Hinge |
| 242 | TTTPAPRPPTPAPTIASQPLSLRPE | CD8 Hinge |
| 243 | IEVMYPPPYLDNEKSNGTIIHVKGKHLCPSPLFPGPSKP | CD28 |
| 244 | ACRPAAGGAVHTRGLDFACDIYIWAPLAGTCGVLLLS | CD8 |
| | LVITLYC | |
| 245 | FWVLVVVGGVLACYSLLVTVAFIIFWV | CD28 |
| 246 | FWVLVVVGGVLACYSLLVTVAFIIFWV | CD28 |
| 247 | RSKRSRLLHSDYMNMTPRRPGPTRKHYQPYAPPRDFA | CD28 |
| | AYRS | |
| 248 | KRGRKKLLYIFKQPFMRPVQTTQEEDGCSCRFPEEEEG | 4-1BB |
| | GCEL | |
| 249 | RVKFSRSADAPAYQQGQNQLYNELNLGRREEYDVLD | CD3zeta |
| | KRRGRDPEMGGKPRRKNPQEGLYNELQKDKMAEAY | |
| | SEIGMKGERRRGKGHDGLYQGLSTATKDTYDALHMQ | |
| | ALPPR | |
| 250 | RVKFSRSADAPAYKQGQNQLYNELNLGRREEYDVLD | CD3zeta |
| | KRRGRDPEMGGKPRRKNPQEGLYNELQKDKMAEAY | |
| | SEIGMKGERRRGKGHDGLYQGLSTATKDTYDALHMQ | |
| | ALPPR | |
| 252 | AVGIGALFLGFLGAAGSTMGAASMTLTVQARQLLSGI | P04578 (512-856) |
| | VQQQNNLLRAIEAQQHLLQLTVWGIKQLQARILAVER | HIV -1 Env (GP41) |
| | YLKDQQLLGIWGCSGKLICTTAVPWNASWSNKSLEQI | |
| | WNHTTWMEW | |
| | DREINNYTSLIHSLIEESQNQQEKNEQELLELDKWASL | |
| | WNWFNITNWLWYIKLFIMIVGGLVGLRIVFAVLSIVN | |
| | RVRQGYSPLSFQTHLPTPRGPDRPEGIEEEGGERDRDR | |
| | SIRLVNGSLALIWDDLRSLCLFSYHRLRDLLLIVTRIVE | |
| | LLGRRGWEALKYWWNLLQYWSQELKNSAVSLLNAT | |
| | AIAVAEGTDRVIEVVQGACRAIRHIPRRIRQGLERILL | |
| 253 | AVGIGALFLGFLGAAGSTMGAASMTLTVQARQLLSDI | AAK08489.2 (512-856) |
| | VQQQNNLLRAIEAQQHLLQLTVWGIKQLQARILAVER | HIV-1 NL4-3 Env (GP41) |
| | YLKDQQLLGIWGCSGKLICTTAVPWNASWSNKSLEQI | |
| | WNNMTWMEWDREINNYTSLIHSLIEESQNQQEKNEQ | |
| | ELLELDKWASLWNWFNITNWLWYIKLFIMIVGGLVG | |
| | LRIVFAVLSIVNRVRQGYSPLSFQTHLPIPRGPDRPEGIE | |
| | EEGGERGRDRSIRLVNGSLALIWDDLRSLCLFSYHRLR | |
| | DLLLIVTRIVELLGRRGWEALKYWWNLLQYWSQELK | |
| | NSAVNLLNATAIAVAEGTDRVIEVLQAAYRAIRHIPRR | |
| | IRQGLERILL | |
| 254 | KKINEGLLDSK | Cytoplasmic tail amino acids |
| 255 | ILHYEKLSKIGLVKGITRKYKIKSNPLTKDIVIKMIPNVS | Hendra virus F Protein, |
| | NVSKCTGTVMENYKSRLTGILSPIKGAIELYNNNTHDL | Without signal sequence |
| | VGDVKLAGVVMAGIAIGIATAAQITAGVALYEAMKN | |
| | ADNINKLKSSIESTNEAVVKLQETAEKTVYVLTALQD | |
| | YINTNLVPTIDQISCKQTELALDLALSKYLSDLLFVFGP | |
| | NLQDPVSNSMTIQAISQAFGGNYETLLRTLGYATEDFD | |
| | DLLESDSIAGQIVYVDLSSYYIIVRVYFPILTEIQQAYV | |
| | QELLPVSFNNDNSEWISIVPNFVLIRNTLISNIEVKYCLI | |
| | TKKSVICNQDYATPMTASVRECLTGSTDKCPRELVVS | |
| | SHVPRFALSGGVLFANCISVTCQCQTTGRAISQSGEQT | |
| | LLMIDNTTCTTVVLGNIIISLGKYLGSINYNSESIAVGPP | |
| | VYTDKVDISSQISSMNQSLQQSKDYIKEAQKILDTVNP | |
| | SLISMLSMIILYVLSIAALCIGLITFISFVIVEKKRGNYSR | |
| | LDDRQVRPVSNGDLYYIGT | |
| 256 | ILHYEKLSKIGLVKGVTRKYKIKSNPLTKDIVIKMIPNV | Nipah virus F Protein, |
| | SNMSQCTGSVMENYKTRLNGILTPIKGALEIYKNNTH | without signal sequence |
| | DLVGDVRLAGVIMAGVAIGIATAAQITAGVALYEAM | |
| | KNADNINKLKSSIESTNEAVVKLQETAEKTVYVLTAL | |
| | QDYINTNLVPTIDKISCKQTELSLDLALSKYLSDLLFVF | |
| | GPNLQDPVSNSMTIQAISQAFGGNYETLLRTLGYATED | |
| | FDDLLESDSITGQIIYVDLSSYYIIVRVYFPILTEIQQAYI | |
| | QELLPVSFNNDNSEWISIVPNFILVRNTLISNIEIGFCLIT | |
| | KRSVICNQDYATPMTNNMRECLTGSTEKCPRELVVSS | |
| | HVPRFALSNGVLFANCISVTCQCQTTGRAISQSGEQTL | |
| | LMIDNTTCPTAVLGNVIISLGKYLGSVNYNSEGIAIGPP | |
| | VFTDKVDISSQISSMNQSLQQSKDYIKEAQRLLDTVNP | |
| | SLISMLSMIILYVLSIASLCIGLITFISFIIVEKKRNTYSRL | |
| | EDRRVRPTSSGDLYYIGT | |
| 257 | TVLIIISYTLFYLNNAAIVGFDFDKLNKIGVVQGRVLN | Cedar Virus F Protein, |
| | YKIKGDPMTKDLVLKFIPNIVNITECVREPLSRYNETV | without signal sequence |
| | RRLLLPIHNMLGLYLNNTNAKMTGLMIAGVIMGGIAI | |
| | GIATAAQITAGFALYEAKKNTENIQKLTDSIMKTQDSI | |
| | DKLTDSVGTSILILNKLQTYINNQLVPNLELLSCRQNKI | |
| | EFDLMLTKYLVDLMTVIGPNINNPVNKDMTIQSLSLLF | |
| | DGNYDIMMSELGYTPQDFLDLIESKSITGQIIYVDMEN | |
| | LYVVIRTYLPTLIEVPDAQIYEFNKITMSSNGGEYLSTIP | |
| | NFILIRGNYMSNIDVATCYMTKASVICNQDYSLPMSQ | |
| | NLRSCYQGETEYCPVEAVIASHSPRFALTNGVIFANCI | |
| | NTICRCQDNGKTITQNINQFVSMIDNSTCNDVMVDKF | |
| | TIKVGKYMGRKDINNINIQIGPQIIIDKVDLSNEINKMN | |
| | QSLKDSIFYLREAKRILDSVNISLISPSVQLFLIIISVLSFII | |
| | LLIIIVYLYCKSKHSYKYNKFIDDPDYYNDYKRERING | |
| | KASKSNNIYYVGD | |
| 258 | IHYDSLSKVGVIKGLTYNYKIKGSPSTKLMVVKLIPNI | Mojiang virus, Tongguan 1 |
| | DSVKNCTQKQYDEYKNLVRKALEPVKMAIDTMLNN | F Protein, without signal |
| | VKSGNNKYRFAGAIMAGVALGVATAATVTAGIALHR | sequence |
| | SNENAQAIANMKSAIQNTNEAVKQLQLANKQTLAVID | |
| | TIRGEINNNIIPVINQLSCDTIGLSVGIRLTQYYSEIITAF | |
| | GPALQNPVNTRITIQAISSVFNGNFDELLKIMGYTSGDL | |
| | YEILHSELIRGNIIDVDVDAGYIALEIEFPNLTLVPNAV | |
| | VQELMPISYNIDGDEWVTLVPRFVLTRTTLLSNIDTSR | |
| | CTITDSSVICDNDYALPMSHELIGCLQGDTSKCAREKV | |
| | VSSYVPKFALSDGLVYANCLNTICRCMDTDTPISQSLG | |
| | ATVSLLDNKRCSVYQVGDVLISVGSYLGDGEYNADN | |
| | VELGPPIVIDKIDIGNQLAGINQTLQEAEDYIEKSEEFLK | |
| | GVNPSIITLGSMVVLYIFMILIAIVSVIALVLSIKLTVKG | |
| | NVVRQQFTYTQHVPSMENINYVSH | |
| 259 | SRALLRETDNYSNGLIVENLVRNCHHPSKNNLNYTKT | Bat Paramyxovirus F |
| | QKRDSTIPYRVEERKGHYPKIKHLIDKSYKHIKRGKRR | Protein, without signal |
| | NGHNGNIITIILLLILILKTQMSEGAIHYETLSKIGLIKGI | sequence |
| | TREYKVKGTPSSKDIVIKLIPNVTGLNKCTNISMENYK | |
| | EQLDKILIPINNIIELYANSTKSAPGNARFAGVIIAGVAL | |
| | GVAAAAQITAGIALHEARQNAERINLLKDSISATNNAV | |
| | AELQEATGGIVNVITGMQDYINTNLVPQIDKLQCSQIK | |
| | TALDISLSQYYSEILTVFGPNLQNPVTTSMSIQAISQSFG | |
| | GNIDLLLNLLGYTANDLLDLLESKSITGQITYINLEHYF | |
| | MVIRVYYPIMTTISNAYVQELIKISFNVDGSEWVSLVP | |
| | SYILIRNSYLSNIDISECLITKNSVICRHDFAMPMSYTLK | |
| | ECLTGDTEKCPREAVVTSYVPRFAISGGVIYANCLSTT | |
| | CQCYQTGKVIAQDGSQTLMMIDNQTCSIVRIEEILISTG | |
| | KYLGSQEYNTMHVSVGNPVFTDKLDITSQISNINQSIE | |
| | QSKFYLDKSKAILDKINLNLIGSVPISILFIIAILSLILSIIT | |
| | FVIVMIIVRRYNKYTPLINSDPSSRRSTIQDVYIIPNPGE | |
| | HSIRSAARSIDRDRD | |
| 260 | GYTFSNYW | CDR-H1 |
| 261 | ILPGSGST | CDR-H2 |
| 262 | ARRGYGYDEGFDY | CDR-H3 |
| 263 | QDINSY | CDR-L1 |
| 264 | RAN | CDR-L2 |
| 265 | LQYDEFPPT | CDR-L3 |
| 266 | NYWIE | CDR-H1 |
| 267 | EILPGSGSTSYNEKFKG | CDR-H2 |
| 268 | RGYGYDEGFDY | CDR-H3 |
| 269 | KASQDINSYLS | CDR-L1 |
| 270 | RANRLVD | CDR-L2 |
| 271 | GYTFSNY | CDR-H1 |
| 272 | LPGSGS | CDR-H2 |
| 273 | QVQLQQSGAELMKPGASVKMSCKATGYTFSNYWIEW | VH |
| | VKQRPGHGLEWIGEILPGSGSTSYNEKFKGKATFTADT | |
| | SSSTAYMQLSSLTSEDSAVYYCARRGYGYDEGFDYW | |
| | GQGSTLTVSS | |
| 274 | DIKMTQSPSSMYASLGERVTITCKASQDINSYLSWFQQ | VI |
| | KPGKSPKTLIYRANRLVDGVPSRFSGSGSGQDYSLTISS | |
| | LEYEDMGIYYCLQYDEFPPTFGAGTKLELKR | |
| 275 | GGGGSGGGGSGGGGS | linker |
| 276 | QVQLQQSGAELMKPGASVKMSCKATGYTFSNYWIEW | scFv |
| | VKQRPGHGLEWIGEILPGSGSTSYNEKFKGKATFTADT | |
| | SSSTAYMQLSSLTSEDSAVYYCARRGYGYDEGFDYW | |
| | GQGSTLTVSSGGGGSGGGGSGGGGSDIKMTQSPSSMY | |
| | ASLGERVTITCKASQDINSYLSWFQQKPGKSPKTLIYR | |
| | ANRLVDGVPSRFSGSGSGQDYSLTISSLEYEDMGIYYC | |
| | LQYDEFPPTFGAGTKLELKR | |
| 277 | GYTFTDYV | CDR-H1 |
| 278 | IYPGSGSS | CDR-H2 |
| 279 | ARPGDLGFAY | CDR-H3 |
| 280 | QSVDYDGDSY | CDR-L1 |
| 281 | AAS | CDR-L2 |
| 282 | QQSNKDPFT | CDR-L3 |
| 283 | DYVIS | CDR-H1 |
| 284 | EIYPGSGSSYYNEKFKG | CDR-H2 |
| 285 | PGDLGFAY | CDR-H3 |
| 286 | KASQSVDYDGDSYMN | CDR-L1 |
| 287 | AASNLES | CDR-L2 |
| 288 | QQSNKDPFT | CDR-L3 |
| 289 | GYTFTDY | CDR-H1 |
| 290 | YPGSGS | CDR-H2 |
| 291 | QVQLQQSGPELVKPGASVKMSCKASGYTFTDYVISW | VH |
| | VRQAPGQGLEWIGEIYPGSGSSYYNEKFKGRATLTAD | |
| | KSSNTAYMQLSSLRSEDSAVYFCARPGDLGFAYWGQ | |
| | GTLVTVSS | |
| 292 | DIVLTQSPSSLAVSLGQRATISCKASQSVDYDGDSYMN | VL |
| | WYQQKPGQPPKLLIYAASNLESGIPARFSGSGSGTDFT | |
| | LTIHPVEEEDAATYYCQQSNKDPFTFGGGTKLELKR | |
| 293 | QVQLQQSGPELVKPGASVKMSCKASGYTFTDYVISW | scFv |
| | VRQAPGQGLEWIGEIYPGSGSSYYNEKFKGRATLTAD | |
| | KSSNTAYMQLSSLRSEDSAVYFCARPGDLGFAYWGQ | |
| | GTLVTVSSGGGGSGGGGSGGGGSDIVLTQSPSSLAVSL | |
| | GQRATISCKASQSVDYDGDSYMNWYQQKPGQPPKLL | |
| | IYAASNLESGIPARFSGSGSGTDFTLTIHPVEEEDAATY | |
| | YCQQSNKDPFTFGGGTKLELKR | |
| 294 | GGTFSSYA | CDR-H1 |
| 295 | INPNSGGT | CDR-H2 |
| 296 | ARDGYSGSYSD | CDR-H3 |
| 297 | QSVLSSSYNKNY | CDR-L1 |
| 298 | WAS | CDR-L2 |
| 299 | QQYYSTPWT | CDR-L3 |
| 300 | SYAIS | CDR-H1 |
| 301 | WINPNSGGTNYAQKFQG | CDR-H2 |
| 302 | DGYSGSYSD | CDR-H3 |
| 303 | KSSQSVLSSSYNKNYLA | CDR-L1 |
| 304 | WASTRES | CDR-L2 |
| 305 | GGTFSSY | CDR-H1 |
| 306 | NPNSGG | CDR-H2 |
| 307 | QVQLVQSGAEVKKPGASVKVSCKASGGTFSSYAISWV | VH |
| | RQAPGQGLEWMGWINPNSGGTNYAQKFQGRVTMTR | |
| | DTSTSTVYMELSSLRSEDTAVYYCARDGYSGSYSDW | |
| | GQGTLVTVSS | |
| 308 | DIVMTQSPDSLAVSLGERATINCKSSQSVLSSSYNKNY | VL |
| | LAWYQQKPGQPPKLLIYWASTRESGVPDRFSGSGSGT | |
| | DFTLTISSLQAEDVAVYYCQQYYSTPWTFGQGTKVEI | |
| | K | |
| 309 | QVQLVQSGAEVKKPGASVKVSCKASGGTFSSYAISWV | scFv |
| | RQAPGQGLEWMGWINPNSGGTNYAQKFQGRVTMTR | |
| | DTSTSTVYMELSSLRSEDTAVYYCARDGYSGSYSDW | |
| | GQGTLVTVSSGGGGSGGGGSGGGGSDIVMTQSPDSLA | |
| | VSLGERATINCKSSQSVLSSSYNKNYLAWYQQKPGQP | |
| | PKLLIYWASTRESGVPDRFSGSGSGTDFTLTISSLQAED | |
| | VAVYYCQQYYSTPWTFGQGTKVEIK | |
| 310 | GYTFTSYD | CDR-H1 |
| 311 | IIPLSGAP | CDR-H2 |
| 312 | ARGALYNWNDGWFDP | CDR-H3 |
| 313 | QDIGDY | CDR-L1 |
| 314 | DAS | CDR-L2 |
| 315 | QQANSFPLT | CDR-L3 |
| 316 | SYDIN | CDR-H1 |
| 317 | GIIPLSGAPNYAHKFQG | CDR-H2 |
| 318 | GALYNWNDGWFDP | CDR-H3 |
| 319 | RASQDIGDYLA | CDR-L1 |
| 320 | DASSLQS | CDR-L2 |
| 321 | GYTFTSY | CDR-H1 |
| 322 | IPLSGA | CDR-H2 |
| 323 | QVQLVQSGAEVKKPGSSVKVSCKASGYTFTSYDINW | VH |
| | VRQAPGQGLEWMGGIIPLSGAPNYAHKFQGRVTITAD | |
| | ESTSTAYMELSSLRSEDTAVYYCARGALYNWNDGWF | |
| | DPWGQGTLVTVSS | |
| 324 | DIQMTQSPSSLSASVGDRVTITCRASQDIGDYLAWYQ | VL |
| | QKPGKAPKLLIYDASSLQSGVPSRFSGSGSGTDFTLTIS | |
| | SLQPEDFATYYCQQANSFPLTFGGGTKVEIK | |
| 325 | QVQLVQSGAEVKKPGSSVKVSCKASGYTFTSYDINW | scFv |
| | VRQAPGQGLEWMGGIIPLSGAPNYAHKFQGRVTITAD | |
| | ESTSTAYMELSSLRSEDTAVYYCARGALYNWNDGWF | |
| | DPWGQGTLVTVSSGGGGSGGGGSGGGGSDIQMTQSP | |
| | SSLSASVGDRVTITCRASQDIGDYLAWYQQKPGKAPK | |
| | LLIYDASSLQSGVPSRFSGSGSGTDFTLTISSLQPEDFAT | |
| | YYCQQANSFPLTFGGGTKVEIK | |
| 326 | GDTFSDHA | CDR-H1 |
| 327 | MNPKIGNT | CDR-H2 |
| 328 | VYDSSGYDAFDI | CDR-H3 |
| 329 | QSVLSTSYNRNF | CDR-L1 |
| 330 | WAS | CDR-L2 |
| 331 | QQYYSTPYT | CDR-L3 |
| 332 | DHAIN | CDR-H1 |
| 333 | WMNPKIGNTGYAQKFQG | CDR-H2 |
| 334 | DSSGYDAFDI | CDR-H3 |
| 335 | KSSQSVLSTSYNRNFLA | CDR-L1 |
| 336 | WASTRQS | CDR-L2 |
| 337 | GDTFSDH | CDR-H1 |
| 338 | NPKIGN | CDR-H2 |
| 339 | QVQLVQSGAEVKKPGASVKVSCKASGDTFSDHAINW | VH |
| | VRQAPGQGLEWMGWMNPKIGNTGYAQKFQGRVTMT | |
| | RDTSTSTVYMELSSLRSEDTAVYYCVYDSSGYDAFDI | |
| | WGQGTTVTVSS | |
| 340 | DIVMTQSPDSLAVSLGERATINCKSSQSVLSTSYNRNF | VL |
| | LAWYQQKPGQPPKLLIYWASTRQSGVPDRFSGSGSGT | |
| | DFTLTISSLQAEDVAVYYCQQYYSTPYTFGQGTKLEIK | |
| 341 | QVQLVQSGAEVKKPGASVKVSCKASGDTFSDHAINW | scFv |
| | VRQAPGQGLEWMGWMNPKIGNTGYAQKFQGRVTMT | |
| | RDTSTSTVYMELSSLRSEDTAVYYCVYDSSGYDAFDI | |
| | WGQGTTVTVSSGGGGSGGGGSGGGGSDIVMTQSPDS | |
| | LAVSLGERATINCKSSQSVLSTSYNRNFLAWYQQKPG | |
| | QPPKLLIYWASTRQSGVPDRFSGSGSGTDFTLTISSLQA | |
| | EDVAVYYCQQYYSTPYTFGQGTKLEIK | |
| 342 | GYSLITHW | CDR-H1 |
| 343 | INPSDGVT | CDR-H2 |
| 344 | AREYYGEGFDY | CDR-H3 |
| 345 | QGISNY | CDR-L1 |
| 346 | SAS | CDR-L2 |
| 347 | QQSYSTPLT | CDR-L3 |
| 348 | THWMH | CDR-H1 |
| 349 | MINPSDGVTYYAQTFQG | CDR-H2 |
| 350 | EYYGEGFDY | CDR-H3 |
| 351 | RASQGISNYLA | CDR-L1 |
| 352 | SASNLQS | CDR-L2 |
| 353 | GYSLITH | CDR-H1 |
| 354 | NPSDGV | CDR-H2 |
| 355 | QVQLVQSGAEVKKPGASVKVSCKASGYSLITHWMH | VH |
| | WVRQAPGQGLEWMGMINPSDGVTYYAQTFQGRVTM | |
| | TRDTSTSTVYMELSSLRSEDTAVYYCAREYYGEGFDY | |
| | WGQGTLVTVSS | |
| 356 | DIQMTQSPSSLSASVGDRVTITCRASQGISNYLAWYQQ | VL |
| | KPGKAPKLLIYSASNLQSGVPSRFSGSGSGTDFTLTISS | |
| | LQPEDFATYYCQQSYSTPLTFGGGTKVEIKR | |
| 357 | QVQLVQSGAEVKKPGASVKVSCKASGYSLITHWMH | scFv |
| | WVRQAPGQGLEWMGMINPSDGVTYYAQTFQGRVTM | |
| | TRDTSTSTVYMELSSLRSEDTAVYYCAREYYGEGFDY | |
| | WGQGTLVTVSSGGGGSGGGGSGGGGSDIQMTQSPSSL | |
| | SASVGDRVTITCRASQGISNYLAWYQQKPGKAPKLLI | |
| | YSASNLQSGVPSRFSGSGSGTDFTLTISSLQPEDFATYY | |
| | CQQSYSTPLTFGGGTKVEIKR | |
| 358 | GFTFSGYW | CDR-H1 |
| 359 | ISPGGGST | CDR-H2 |
| 360 | ASSLTATHTYEYDY | CDR-H3 |
| 361 | GYWMY | CDR-H1 |
| 362 | AISPGGGSTYYPDSVKG | CDR-H2 |
| 363 | SLTATHTYEYDY | CDR-H3 |
| 364 | GFTFSGY | CDR-H1 |
| 365 | SPGGGS | CDR-H2 |
| 366 | EVQLVESGGGLVQSGGSLRLSCAASGFTFSGYWMYW | VHH |
| | VRQAPGKGLEWVSAISPGGGSTYYPDSVKGRFTISRD | |
| | NAKNTLYLQMNSLEPEDTALYYCASSLTATHTYEYD | |
| | YWGQGTQVTVSS | |
| 367 | TTTPAPRPPTPAPTIASQPLSLRPEACRPAAGGAVHTRG | CD8α hinge domain |
| | LDFACD | |
| 368 | IYIWAPLAGTCGVLLLSLVITLYC | CD8α transmembrane |
| | | domain |
| 369 | QVQLVQSGAEVKKPGASVKVSCKASGGTFSSYAISWV | CD8_1 VH |
| | RQAPGQGLEWMGIIDPSDGNTNYAQNFQGRVTMTRD | |
| | TSTSTVYMELSSLRSEDTAVYYCAKERAAAGYYYYM | |
| | DVWGQGTTVTVSS | |
| 370 | DIQMTQSPSSLSASVGDRVTITCRASQSISSYLNWYQQ | CD8_1 VL |
| | KPGKAPKLLIYAASSLQSGVPSRFSGSGSGTDFTLTISS | |
| | LQPEDFATYYCQQSYSTPLTFGGGTKVEIKR | |
| 371 | QVQLVQSGAEVKKPGASVKVSCKASGYTFTDYYIQW | CD8_2 VH |
| | VRQAPGQGLEWMGWINPNSGGTSYAQKFQGRVTMT | |
| | RDTSTSTVYMELSSLRSEDTAVYYCAKEGDYYYGMD | |
| | AWGQGTMVTVSS | |
| 372 | DIVMTQSPLSLPVTPGEPASISCRSSQSLLHSNGYNYLD | CD8_2 VL |
| | WYLQKPGQSPQLLIYLGSNRASGVPDRFSGSGSGTDFT | |
| | LKISRVEAEDVGVYYCMQGLQTPHTFGQGTKVEIKR | |
| 373 | QVQLVQSGAEVKKPGASVKVSCKASGYTFTSYYMH | CD8_3 VH |
| | WVRQAPGQGLEWMGGFDPEDGETIYAQKFQGRVTM | |
| | TRDTSTSTVYMELSSLRSEDTAVYYCARDQGWGMDV | |
| | WGQGTTVTVSS | |
| 374 | DIQMTQSPSSLSASVGDRVTITCRASQSISSYLNWYQQ | CD8_3 VL |
| | KPGKAPKLLIYAASSLQSGVPSRFSGSGSGTDFTLTISS | |
| | LQPEDFATYYCQQTYSTPYTFGQGTKLEIKR | |
| 375 | QVQLVQSGAEVKKPGASVKVSCKASGYTFTNHYMH | CD8_4 VH |
| | WVRQAPGQGLEWMGWMNPNSGNTGYAQKFQGRVT | |
| | MTRDTSTSTVYMELSSLRSEDTAVYYCASSESGSDLD | |
| | YWGQGTLVTVSS | |
| 376 | DIQMTQSPSSLSASVGDRVTITCRASQTIGNYVNWYQ | CD8_4 VL |
| | QKPGKAPKLLIYGASNLHTGVPSRFSGSGSGTDFTLTIS | |
| | SLQPEDFATYYCQQTYSAPLTFGGGTKVEIKR | |
| 377 | QVQLVESGGGLVQAGGSLRLSCAASGRTFSGYVMGW | VHH |
| | FRQAPGKQRKFVAAISRGGLSTSYADSVKGRFTISRDN | |
| | AKNTVFLQMNTLKPEDTAVYYCAADRSDLYEITAAS | |
| | NIDSWGQGTLVTVSS | |
| 378 | MALPVTALLLPLALLLHAARP | CD8α signal peptide |
| 379 | METDTLLLWVLLLWVPGSTG | IgK signal peptide |
| 380 | MLLLVTSLLLCELPHPAFLLIP | GMCSFR-α (CSF2RA) |
| | | signal peptide |
| 381 | TTTPAPRPPTPAPTIASQPLSLRPEACRPAAGGAVHTRG | CD8α hinge domain |
| | LDFACD | |
| 382 | IEVMYPPPYLDNEKSNGTIIHVKGKHLCPSPLFPGPSKP | CD28 hinge domain |
| 383 | AAAIEVMYPPPYLDNEKSNGTIIHVKGKHLCPSPLFPG | CD28 hinge domain |
| | PSKP | |
| 384 | ESKYGPPCPPCP | IgG4 hinge domain |
| 385 | ESKYGPPCPSCP | IgG4 hinge domain |
| 386 | ESKYGPPCPPCPAPEFLGGPSVFLFPPKPKDTLMISRTP | IgG4 hinge-CH2-CH3 |
| | EVTCVVVDVSQEDPEVQFNWYVDGVEVHNAKTKPRE | domain |
| | EQFNSTYRVVSVLTVLHQDWLNGKEYKCKVSNKGLP | |
| | SSIEKTISKAKGQPREPQVYTLPPSQEEMTKNQVSLTC | |
| | LVKGFYPSDIAVEWESNGQPENNYKTTPPVLDSDGSFF | |
| | LYSRLTVDKSRWQEGNVFSCSVMHEALHNHYTQKSL | |
| | SLSLGK | |
| 387 | IYIWAPLAGTCGVLLLSLVITLYC | CD8α transmembrane |
| | | domain |
| 388 | FWVLVVVGGVLACYSLLVTVAFIIFWV | CD28 transmembrane |
| | | domain |
| 389 | MFWVLVVVGGVLACYSLLVTVAFIIFWV | CD28 transmembrane |
| | | domain |
| 390 | KRGRKKLLYIFKQPFMRPVQTTQEEDGCSCRFPEEEEG | 4-1BB costimulatory domain |
| | GCEL | |
| 391 | RSKRSRLLHSDYMNMTPRRPGPTRKHYQPYAPPRDFA | CD28 costimulatory domain |
| | AYRS | |
| 392 | RVKFSRSADAPAYQQGQNQLYNELNLGRREEYDVLD | CD3ζ signaling domain |
| | KRRGRDPEMGGKPRRKNPQEGLYNELQKDKMAEAY | |
| | SEIGMKGERRRGKGHDGLYQGLSTATKDTYDALHMQ | |
| | ALPPR | |
| 393 | RVKFSRSADAPAYKQGQNQLYNELNLGRREEYDVLD | CD3ζ signaling domain |
| | KRRGRDPEMGGKPRRKNPQEGLYNELQKDKMAEAY | (with Q to K mutation at |
| | SEIGMKGERRRGKGHDGLYQGLSTATKDTYDALHMQ | position 14) |
| | ALPPR | |
| 394 | DIQMTQTTSSLSASLGDRVTISCRASQDISKYLNWYQQ | Anti-CD19 FMC63 scFv |
| | KPDGTVKLLIYHTSRLHSGVPSRFSGSGSGTDYSLTISN | entire sequence, with |
| | LEQEDIATYFCQQGNTLPYTFGGGTKLEITGSTSGSGK | Whitlow linker |
| | PGSGEGSTKGEVKLQESGPGLVAPSQSLSVTCTVSGVS | |
| | LPDYGVSWIRQPPRKGLEWLGVIWGSETTYYNSALKS | |
| | RLTIIKDNSKSQVFLKMNSLQTDDTAIYYCAKHYYYG | |
| | GSYAMDYWGQGTSVTVSS | |
| 395 | DIQMTQTTSSLSASLGDRVTISCRASQDISKYLNWYQQ | Anti-CD19 FMC63 scFv |
| | KPDGTVKLLIYHTSRLHSGVPSRFSGSGSGTDYSLTISN | light chain variable region |
| | LEQEDIATYFCQQGNTLPYTFGGGTKLEIT | |
| 396 | QDISKY | Anti-CD19 FMC63 scFv |
| | | light chain CDR1 |
| 397 | HTS | Anti-CD19 FMC63 scFv |
| | | light chain CDR2 |
| 398 | QQGNTLPYT | Anti-CD19 FMC63 scFv |
| | | light chain CDR3 |
| 399 | GSTSGSGKPGSGEGSTKG | Whitlow linker |
| 400 | EVKLQESGPGLVAPSQSLSVTCTVSGVSLPDYGVSWIR | Anti-CD19 FMC63 scFv |
| | QPPRKGLEWLGVIWGSETTYYNSALKSRLTIIKDNSKS | heavy chain variable region |
| | QVFLKMNSLQTDDTAIYYCAKHYYYGGSYAMDYWG | |
| | QGTSVTVSS | |
| 401 | GVSLPDYG | Anti-CD19 FMC63 scFv |
| | | heavy chain CDR1 |
| 402 | IWGSETT | Anti-CD19 FMC63 scFv |
| | | heavy chain CDR2 |
| 403 | AKHYYYGGSYAMDY | Anti-CD19 FMC63 scFv |
| | | heavy chain CDR3 |
| 404 | DIQMTQTTSSLSASLGDRVTISCRASQDISKYLNWYQQ | Anti-CD19 FMC63 scFv |
| | KPDGTVKLLIYHTSRLHSGVPSRFSGSGSGTDYSLTISN | entire sequence, with 3xG4S |
| | LEQEDIATYFCQQGNTLPYTFGGGTKLEITGGGGSGG | linker |
| | GGSGGGGSEVKLQESGPGLVAPSQSLSVTCTVSGVSLP | |
| | DYGVSWIRQPPRKGLEWLGVIWGSETTYYNSALKSRL | |
| | TIIKDNSKSQVFLKMNSLQTDDTAIYYCAKHYYYGGS | |
| | YAMDYWGQGTSVTVSS | |
| 405 | GGGGSGGGGSGGGGS | 3xG₄S linker |
| 406 | atggccttaccagtgaccgccttgctcctgccgctggccttgctg | Exemplary CD19 CAR |
| | ctccacgccgccaggccggacatccagatgacacagactacatcc | nucleotide sequence |
| | tccctgtctgcctctctgggagacagagtcaccatcagttgcagg | |
| | gcaagtcaggacattagtaaatatttaaattggtatcagcagaaa | |
| | ccagatggaactgttaaactcctgatctaccatacatcaagatta | |
| | cactcaggagtcccatcaaggttcagtggcagtgggtctggaaca | |
| | gattattctctcaccattagcaacctggagcaagaagatattgcc | |
| | acttacttttgccaacagggtaatacgcttccgtacacgttcgga | |
| | ggggggaccaagctggagatcacaggctccacctctggatccggc | |
| | aagcccggatctggcgagggatccaccaagggcgaggtgaaactg | |
| | caggagtcaggacctggcctggtggcgccctcacagagcctgtcc | |
| | gtcacatgcactgtctcaggggtctcattacccgactatggtgta | |
| | agctggattcgccagcctccacgaaagggtctggagtggctggga | |
| | gtaatatggggtagtgaaaccacatactataattcagctctcaaa | |
| | tccagactgaccatcatcaaggacaactccaagagccaagttttc | |
| | ttaaaaatgaacagtctgcaaactgatgacacagccatttactac | |
| | tgtgccaaacattattactacggtggtagctatgctatggactac | |
| | tggggccaaggaacctcagtcaccgtctcctcaaccacgacgcca | |
| | gcgccgcgaccaccaacaccggcgcccaccatcgcgtcgcagccc | |
| | ctgtccctgcgcccagaggcgtgccggccagcggggggggcgcag | |
| | tgcacacgagggggctggacttcgcctgtgatatctacatctggg | |
| | cgcccttggccgggacttgtggggtccttctcctgtcactggtta | |
| | tcaccctttactgcaaacggggcagaaagaaactcctgtatatat | |
| | tcaaacaaccatttatgagaccagtacaaactactcaagaggaag | |
| | atggctgtagctgccgatttccagaagaagaagaaggaggatgtg | |
| | aactgagagtgaagttcagcaggagcgcagacgcccccgcgtacc | |
| | agcagggccagaaccagctctataacgagctcaatctaggacgaa | |
| | gagaggagtacgatgttttggacaagagacgtggccgggaccctg | |
| | agatggggggaaagccgagaaggaagaaccctcaggaaggcctgt | |
| | acaatgaactgcagaaagataagatggcggaggcctacagtgaga | |
| | ttgggatgaaaggcgagcgccggaggggcaaggggcacgatggcc | |
| | tttaccagggtctcagtacagccaccaaggacacctacgacgccc | |
| | ttcacatgcaggccctgccccctcgc | |
| 407 | MALPVTALLLPLALLLHAARPDIQMTQTTSSLSASLGD | Exemplary CD19 CAR |
| | RVTISCRASQDISKYLNWYQQKPDGTVKLLIYHTSRLH | amino acid sequence |
| | SGVPSRFSGSGSGTDYSLTISNLEQEDIATYFCQQGNTL | |
| | PYTFGGGTKLEITGSTSGSGKPGSGEGSTKGEVKLQES | |
| | GPGLVAPSQSLSVTCTVSGVSLPDYGVSWIRQPPRKGL | |
| | EWLGVIWGSETTYYNSALKSRLTIIKDNSKSQVFLKM | |
| | NSLQTDDTAIYYCAKHYYYGGSYAMDYWGQGTSVT | |
| | VSSTTTPAPRPPTPAPTIASQPLSLRPEACRPAAGGAVH | |
| | TRGLDFACDIYIWAPLAGTCGVLLLSLVITLYCKRGRK | |
| | KLLYIFKQPFMRPVQTTQEEDGCSCRFPEEEEGGCELR | |
| | VKFSRSADAPAYQQGQNQLYNELNLGRREEYDVLDK | |
| | RRGRDPEMGGKPRRKNPQEGLYNELQKDKMAEAYSE | |
| | IGMKGERRRGKGHDGLYQGLSTATKDTYDALHMQA | |
| | LPPR | |
| 408 | atggccttaccagtgaccgccttgctcctgccgctggccttgctg | Tisagenlecleucel CD19 CAR |
| | ctccacgccgccaggccggacatccagatgacacagactacatcc | nucleotide sequence |
| | tccctgtctgcctctctgggagacagagtcaccatcagttgcagg | |
| | gcaagtcaggacattagtaaatatttaaattggtatcagcagaaa | |
| | ccagatggaactgttaaactcctgatctaccatacatcaagatta | |
| | cactcaggagtcccatcaaggttcagtggcagtgggtctggaaca | |
| | gattattctctcaccattagcaacctggagcaagaagatattgcc | |
| | acttacttttgccaacagggtaatacgcttccgtacacgttcgga | |
| | ggggggaccaagctggagatcacaggtggcggtggctcgggcggt | |
| | ggtgggtcgggtggcggcggatctgaggtgaaactgcaggagtca | |
| | ggacctggcctggtggcgccctcacagagcctgtccgtcacatgc | |
| | actgtctcaggggtctcattacccgactatggtgtaagctggatt | |
| | cgccagcctccacgaaagggtctggagtggctgggagtaatatgg | |
| | ggtagtgaaaccacatactataattcagctctcaaatccagactg | |
| | accatcatcaaggacaactccaagagccaagttttcttaaaaatg | |
| | aacagtctgcaaactgatgacacagccatttactactgtgccaaa | |
| | cattattactacggtggtagctatgctatggactactggggccaa | |
| | ggaacctcagtcaccgtctcctcaaccacgacgccagcgccgcga | |
| | ccaccaacaccggcgcccaccatcgcgtcgcagcccctgtccctg | |
| | cgcccagaggcgtgccggccagcggcggggggcgcagtgcacacg | |
| | agggggctggacttcgcctgtgatatctacatctgggcgcccttg | |
| | gccgggacttgtggggtccttctcctgtcactggttatcaccctt | |
| | tactgcaaacggggcagaaagaaactcctgtatatattcaaacaa | |
| | ccatttatgagaccagtacaaactactcaagaggaagatggctgt | |
| | agctgccgatttccagaagaagaagaaggaggatgtgaactgaga | |
| | gtgaagttcagcaggagcgcagacgcccccgcgtacaagcagggc | |
| | cagaaccagctctataacgagctcaatctaggacgaagagaggag | |
| | tacgatgttttggacaagagacgtggccgggaccctgagatgggg | |
| | ggaaagccgagaaggaagaaccctcaggaaggcctgtacaatgaa | |
| | ctgcagaaagataagatggcggaggcctacagtgagattgggatg | |
| | aaaggcgagcgccggaggggcaaggggcacgatggcctttaccag | |
| | ggtctcagtacagccaccaaggacacctacgacgcccttcacatg | |
| | caggccctgccccctcgc | |
| 409 | MALPVTALLLPLALLLHAARPDIQMTQTTSSLSASLGD | Tisagenlecleucel CD19 CAR |
| | RVTISCRASQDISKYLNWYQQKPDGTVKLLIYHTSRLH | amino acid sequence |
| | SGVPSRFSGSGSGTDYSLTISNLEQEDIATYFCQQGNTL | |
| | PYTFGGGTKLEITGGGGSGGGGSGGGGSEVKLQESGP | |
| | GLVAPSQSLSVTCTVSGVSLPDYGVSWIRQPPRKGLE | |
| | WLGVIWGSETTYYNSALKSRLTIIKDNSKSQVFLKMN | |
| | SLQTDDTAIYYCAKHYYYGGSYAMDYWGQGTSVTV | |
| | SSTTTPAPRPPTPAPTIASQPLSLRPEACRPAAGGAVHT | |
| | RGLDFACDIYIWAPLAGTCGVLLLSLVITLYCKRGRKK | |
| | LLYIFKQPFMRPVQTTQEEDGCSCRFPEEEEGGCELRV | |
| | KFSRSADAPAYKQGQNQLYNELNLGRREEYDVLDKR | |
| | RGRDPEMGGKPRRKNPQEGLYNELQKDKMAEAYSEI | |
| | GMKGERRRGKGHDGLYQGLSTATKDTYDALHMQAL | |
| | PPR | |
| 410 | atgctgctgctggtgaccagcctgctgctgtgcgagctgccccac | Lisocabtagene maraleucel |
| | cccgcctttctgctgatccccgacatccagatgacccagaccacc | CD19 CAR nucleotide |
| | tccagcctgagcgccagcctgggcgaccgggtgaccatcagctgc | sequence |
| | cgggccagccaggacatcagcaagtacctgaactggtatcagcag | |
| | aagcccgacggcaccgtcaagctgctgatctaccacaccagccgg | |
| | ctgcacagcggcgtgcccagccggtttagcggcagcggctccggc | |
| | accgactacagcctgaccatctccaacctggaacaggaagatatc | |
| | gccacctacttttgccagcagggcaacacactgccctacaccttt | |
| | ggcggcggaacaaagctggaaatcaccggcagcacctccggcagc | |
| | ggcaagcctggcagcggcgagggcagcaccaagggcgaggtgaag | |
| | ctgcaggaaagcggccctggcctggtggcccccagccagagcctg | |
| | agcgtgacctgcaccgtgagcggcgtgagcctgcccgactacggc | |
| | gtgagctggatccggcagccccccaggaagggcctggaatggctg | |
| | ggcgtgatctggggcagcgagaccacctactacaacagcgccctg | |
| | aagagccggctgaccatcatcaaggacaacagcaagagccaggtg | |
| | ttcctgaagatgaacagcctgcagaccgacgacaccgccatctac | |
| | tactgcgccaagcactactactacggcggcagctacgccatggac | |
| | tactggggccagggcaccagcgtgaccgtgagcagcgaatctaag | |
| | tacggaccgccctgccccccttgccctatgttctgggtgctggtg | |
| | gtggtcggaggcgtgctggcctgctacagcctgctggtcaccgtg | |
| | gccttcatcatcttttgggtgaaacggggcagaaagaaactcctg | |
| | tatatattcaaacaaccatttatgagaccagtacaaactactcaa | |
| | gaggaagatggctgtagctgccgatttccagaagaagaagaagga | |
| | ggatgtgaactgcgggtgaagttcagcagaagcgccgacgcccct | |
| | gcctaccagcagggccagaatcagctgtacaacgagctgaacctg | |
| | ggcagaagggaagagtacgacgtcctggataagcggagaggccgg | |
| | gaccctgagatgggcggcaagcctcggcggaagaacccccaggaa | |
| | ggcctgtataacgaactgcagaaagacaagatggccgaggcctac | |
| | agcgagatcggcatgaagggcgagcggaggcggggcaagggccac | |
| | gacggcctgtatcagggcctgtccaccgccaccaaggatacctac | |
| | gacgccctgcacatgcaggccctgcccccaagg | |
| 411 | MLLLVTSLLLCELPHPAFLLIPDIQMTQTTSSLSASLGD | Lisocabtagene maraleucel |
| | RVTISCRASQDISKYLNWYQQKPDGTVKLLIYHTSRLH | CD19 CAR amino acid |
| | SGVPSRFSGSGSGTDYSLTISNLEQEDIATYFCQQGNTL | sequence |
| | PYTFGGGTKLEITGSTSGSGKPGSGEGSTKGEVKLQES | |
| | GPGLVAPSQSLSVTCTVSGVSLPDYGVSWIRQPPRKGL | |
| | EWLGVIWGSETTYYNSALKSRLTIIKDNSKSQVFLKM | |
| | NSLQTDDTAIYYCAKHYYYGGSYAMDYWGQGTSVT | |
| | VSSESKYGPPCPPCPMFWVLVVVGGVLACYSLLVTVA | |
| | FIIFWVKRGRKKLLYIFKQPFMRPVQTTQEEDGCSCRF | |
| | PEEEEGGCELRVKFSRSADAPAYQQGQNQLYNELNLG | |
| | RREEYDVLDKRRGRDPEMGGKPRRKNPQEGLYNELQ | |
| | KDKMAEAYSEIGMKGERRRGKGHDGLYQGLSTATKD | |
| | TYDALHMQALPPR | |
| 412 | atgcttctcctggtgacaagccttctgctctgtgagttaccacac | Axicabtagene ciloleucel |
| | ccagcattcctcctgatcccagacatccagatgacacagactaca | CD19 CAR nucleotide |
| | tcctccctgtctgcctctctgggagacagagtcaccatcagttgc | sequence |
| | agggcaagtcaggacattagtaaatatttaaattggtatcagcag | |
| | aaaccagatggaactgttaaactcctgatctaccatacatcaaga | |
| | ttacactcaggagtcccatcaaggttcagtggcagtgggtctgga | |
| | acagattattctctcaccattagcaacctggagcaagaagatatt | |
| | gccacttacttttgccaacagggtaatacgcttccgtacacgttc | |
| | ggaggggggactaagttggaaataacaggctccacctctggatcc | |
| | ggcaagcccggatctggcgagggatccaccaagggcgaggtgaaa | |
| | ctgcaggagtcaggacctggcctggtggcgccctcacagagcctg | |
| | tccgtcacatgcactgtctcaggggtctcattacccgactatggt | |
| | gtaagctggattcgccagcctccacgaaagggtctggagtggctg | |
| | ggagtaatatggggtagtgaaaccacatactataattcagctctc | |
| | aaatccagactgaccatcatcaaggacaactccaagagccaagtt | |
| | ttcttaaaaatgaacagtctgcaaactgatgacacagccatttac | |
| | tactgtgccaaacattattactacggtggtagctatgctatggac | |
| | tactggggtcaaggaacctcagtcaccgtctcctcagcggccgca | |
| | attgaagttatgtatcctcctccttacctagacaatgagaagagc | |
| | aatggaaccattatccatgtgaaagggaaacacctttgtccaagt | |
| | cccctatttcccggaccttctaagcccttttgggtgctggtggtg | |
| | gttgggggagtcctggcttgctatagcttgctagtaacagtggcc | |
| | tttattattttctgggtgaggagtaagaggagcaggctcctgcac | |
| | agtgactacatgaacatgactccccgccgccccgggcccacccgc | |
| | aagcattaccagccctatgccccaccacgcgacttcgcagcctat | |
| | cgctccagagtgaagttcagcaggagcgcagacgcccccgcgtac | |
| | cagcagggccagaaccagctctataacgagctcaatctaggacga | |
| | agagaggagtacgatgttttggacaagagacgtggccgggaccct | |
| | gagatggggggaaagccgagaaggaagaaccctcaggaaggcctg | |
| | tacaatgaactgcagaaagataagatggcggaggcctacagtgag | |
| | attgggatgaaaggcgagcgccggaggggcaaggggcacgatggc | |
| | ctttaccagggtctcagtacagccaccaaggacacctacgacgcc | |
| | cttcacatgcaggccctgccccctcgc | |
| 413 | MLLLVTSLLLCELPHPAFLLIPDIQMTQTTSSLSASLGD | Axicabtagene ciloleucel |
| | RVTISCRASQDISKYLNWYQQKPDGTVKLLIYHTSRLH | CD19 CAR amino acid |
| | SGVPSRFSGSGSGTDYSLTISNLEQEDIATYFCQQGNTL | sequence |
| | PYTFGGGTKLEITGSTSGSGKPGSGEGSTKGEVKLQES | |
| | GPGLVAPSQSLSVTCTVSGVSLPDYGVSWIRQPPRKGL | |
| | EWLGVIWGSETTYYNSALKSRLTIIKDNSKSQVFLKM | |
| | NSLQTDDTAIYYCAKHYYYGGSYAMDYWGQGTSVT | |
| | VSSAAAIEVMYPPPYLDNEKSNGTIIHVKGKHLCPSPL | |
| | FPGPSKPFWVLVVVGGVLACYSLLVTVAFIIFWVRSK | |
| | RSRLLHSDYMNMTPRRPGPTRKHYQPYAPPRDFAAY | |
| | RSRVKFSRSADAPAYQQGQNQLYNELNLGRREEYDV | |
| | LDKRRGRDPEMGGKPRRKNPQEGLYNELQKDKMAE | |
| | AYSEIGMKGERRRGKGHDGLYQGLSTATKDTYDALH | |
| | MQALPPR | |
| 414 | DIVLTQSPAILSASPGEKVTMTCRASSSVNYMDWYQK | Anti-CD20 Leu16 scFv |
| | KPGSSPKPWIYATSNLASGVPARFSGSGSGTSYSLTISR | entire sequence, with |
| | VEAEDAATYYCQQWSFNPPTFGGGTKLEIKGSTSGSG | Whitlow linker |
| | KPGSGEGSTKGEVQLQQSGAELVKPGASVKMSCKAS | |
| | GYTFTSYNMHWVKQTPGQGLEWIGAIYPGNGDTSYN | |
| | QKFKGKATLTADKSSSTAYMQLSSLTSEDSADYYCAR | |
| | SNYYGSSYWFFDVWGAGTTVTVSS | |
| 415 | DIVLTQSPAILSASPGEKVTMTCRASSSVNYMDWYQK | Anti-CD20 Leu16 scFv light |
| | KPGSSPKPWIYATSNLASGVPARFSGSGSGTSYSLTISR | chain variable region |
| | VEAEDAATYYCQQWSFNPPTFGGGTKLEIK | |
| 416 | RASSSVNYMD | Anti-CD20 Leu16 scFv light |
| | | chain CDR1 |
| 417 | ATSNLAS | Anti-CD20 Leu16 scFv light |
| | | chain CDR2 |
| 418 | QQWSFNPPT | Anti-CD20 Leu16 scFv light |
| | | chain CDR3 |
| 419 | EVQLQQSGAELVKPGASVKMSCKASGYTFTSYNMHW | Anti-CD20 Leu16 scFv |
| | VKQTPGQGLEWIGAIYPGNGDTSYNQKFKGKATLTA | heavy chain |
| | DKSSSTAYMQLSSLTSEDSADYYCARSNYYGSSYWFF | |
| | DVWGAGTTVTVSS | |
| 420 | SYNMH | Anti-CD20 Leu16 scFv |
| | | heavy chain CDR1 |
| 421 | AIYPGNGDTSYNQKFKG | Anti-CD20 Leu16 scFv |
| | | heavy chain CDR2 |
| 422 | SNYYGSSYWFFDV | Anti-CD20 Leu16 scFv |
| | | heavy chain CDR3 |
| 423 | QVQLQQSGPGLVKPSQTLSLTCAISGDSVSSNSAAWN | Anti-CD22 m971 scFv entire |
| | WIRQSPSRGLEWLGRTYYRSKWYNDYAVSVKSRITIN | sequence, with 3xG4S linker |
| | PDTSKNQFSLQLNSVTPEDTAVYYCAREVTGDLEDAF | |
| | DIWGQGTMVTVSSGGGGSGGGGSGGGGSDIQMTQSP | |
| | SSLSASVGDRVTITCRASQTIWSYLNWYQQRPGKAPN | |
| | LLIYAASSLQSGVPSRFSGRGSGTDFTLTISSLQAEDFA | |
| | TYYCQQSYSIPQTFGQGTKLEIK | |
| 424 | QVQLQQSGPGLVKPSQTLSLTCAISGDSVSSNSAAWN | Anti-CD22 m971 scFv |
| | WIRQSPSRGLEWLGRTYYRSKWYNDYAVSVKSRITIN | heavy chain variable region |
| | PDTSKNQFSLQLNSVTPEDTAVYYCAREVTGDLEDAF | |
| | DIWGQGTMVTVSS | |
| 425 | GDSVSSNSAA | Anti-CD22 m971 scFv |
| | | heavy chain CDR1 |
| 426 | TYYRSKWYN | Anti-CD22 m971 scFv |
| | | heavy chain CDR2 |
| 427 | AREVTGDLEDAFDI | Anti-CD22 m971 scFv |
| | | heavy chain CDR3 |
| 428 | DIQMTQSPSSLSASVGDRVTITCRASQTIWSYLNWYQ | Anti-CD22 m971 scFv light |
| | QRPGKAPNLLIYAASSLQSGVPSRFSGRGSGTDFTLTIS | chain |
| | SLQAEDFATYYCQQSYSIPQTFGQGTKLEIK | |
| 429 | QTIWSY | Anti-CD22 m971 scFv light |
| | | chain CDR1 |
| 430 | AAS | Anti-CD22 m971 scFv light |
| | | chain CDR2 |
| 431 | QQSYSIPQT | Anti-CD22 m971 scFv light |
| | | chain CDR3 |
| 432 | QVQLQQSGPGMVKPSQTLSLTCAISGDSVSSNSVAWN | Anti-CD22 m971-L7 scFv |
| | WIRQSPSRGLEWLGRTYYRSTWYNDYAVSMKSRITIN | entire sequence, with 3xG4S |
| | PDTNKNQFSLQLNSVTPEDTAVYYCAREVTGDLEDAF | linker |
| | DIWGQGTMVTVSSGGGGSGGGGSGGGGSDIQMIQSPS | |
| | SLSASVGDRVTITCRASQTIWSYLNWYRQRPGEAPNL | |
| | LIYAASSLQSGVPSRFSGRGSGTDFTLTISSLQAEDFAT | |
| | YYCQQSYSIPQTFGQGTKLEIK | |
| 433 | QVQLQQSGPGMVKPSQTLSLTCAISGDSVSSNSVAWN | Anti-CD22 m971-L7 scFv |
| | WIRQSPSRGLEWLGRTYYRSTWYNDYAVSMKSRITIN | heavy chain variable region |
| | PDTNKNQFSLQLNSVTPEDTAVYYCAREVTGDLEDAF | |
| | DIWGQGTMVTVSS | |
| 434 | GDSVSSNSVA | Anti-CD22 m971-L7 scFv |
| | | heavy chain CDR1 |
| 435 | TYYRSTWYN | Anti-CD22 m971-L7 scFv |
| | | heavy chain CDR2 |
| 436 | AREVTGDLEDAFDI | Anti-CD22 m971-L7 scFv |
| | | heavy chain CDR3 |
| 437 | DIQMIQSPSSLSASVGDRVTITCRASQTIWSYLNWYRQ | Anti-CD22 m971-L7 scFv |
| | RPGEAPNLLIYAASSLQSGVPSRFSGRGSGTDFTLTISS | light chain variable region |
| | LQAEDFATYYCQQSYSIPQTFGQGTKLEIK | |
| 438 | QTIWSY | Anti-CD22 m971-L7 scFv |
| | | light chain CDR1 |
| 439 | AAS | Anti-CD22 m971-L7 scFv |
| | | light chain CDR2 |
| 440 | QQSYSIPQT | Anti-CD22 m971-L7 scFv |
| | | light chain CDR3 |
| 441 | DIVLTQSPASLAMSLGKRATISCRASESVSVIGAHLIH | Anti-BCMA C11D5.3 scFv |
| | WYQQKPGQPPKLLIYLASNLETGVPARFSGSGSGTDFT | entire sequence, with |
| | LTIDPVEEDDVAIYSCLQSRIFPRTFGGGTKLEIKGSTS | Whitlow linker |
| | GSGKPGSGEGSTKGQIQLVQSGPELKKPGETVKISCKA | |
| | SGYTFTDYSINWVKRAPGKGLKWMGWINTETREPAY | |
| | AYDFRGRFAFSLETSASTAYLQINNLKYEDTATYFCAL | |
| | DYSYAMDYWGQGTSVTVSS | |
| 442 | DIVLTQSPASLAMSLGKRATISCRASESVSVIGAHLIH | Anti-BCMA C11D5.3 scFv |
| | WYQQKPGQPPKLLIYLASNLETGVPARFSGSGSGTDFT | light chain variable region |
| | LTIDPVEEDDVAIYSCLQSRIFPRTFGGGTKLEIK | |
| 443 | RASESVSVIGAHLIH | Anti-BCMA C11D5.3 scFv |
| | | light chain CDR1 |
| 444 | LASNLET | Anti-BCMA C11D5.3 scFv |
| | | light chain CDR2 |
| 445 | LQSRIFPRT | Anti-BCMA C11D5.3 scFv |
| | | light chain CDR3 |
| 446 | QIQLVQSGPELKKPGETVKISCKASGYTFTDYSINWVK | Anti-BCMA C11D5.3 scFv |
| | RAPGKGLKWMGWINTETREPAYAYDFRGRFAFSLETS | heavy chain variable region |
| | ASTAYLQINNLKYEDTATYFCALDYSYAMDYWGQGT | |
| | SVTVSS | |
| 447 | DYSIN | Anti-BCMA C11D5.3 scFv |
| | | heavy chain CDR1 |
| 448 | WINTETREPAYAYDFRG | Anti-BCMA C11D5.3 scFv |
| | | heavy chain CDR2 |
| 449 | DYSYAMDY | Anti-BCMA C11D5.3 scFv |
| | | heavy chain CDR3 |
| 450 | DIVLTQSPPSLAMSLGKRATISCRASESVTILGSHLIYW | Anti-BCMA C12A3.2 scFv |
| | YQQKPGQPPTLLIQLASNVQTGVPARFSGSGSRTDFTL | entire sequence, with |
| | TIDPVEEDDVAVYYCLQSRTIPRTFGGGTKLEIKGSTS | Whitlow linker |
| | GSGKPGSGEGSTKGQIQLVQSGPELKKPGETVKISCKA | |
| | SGYTFRHYSMNWVKQAPGKGLKWMGRINTESGVPIY | |
| | ADDFKGRFAFSVETSASTAYLVINNLKDEDTASYFCSN | |
| | DYLYSLDFWGQGTALTVSS | |
| 451 | DIVLTQSPPSLAMSLGKRATISCRASESVTILGSHLIYW | Anti-BCMA C12A3.2 scFv |
| | YQQKPGQPPTLLIQLASNVQTGVPARFSGSGSRTDFTL | light chain variable region |
| | TIDPVEEDDVAVYYCLQSRTIPRTFGGGTKLEIK | |
| 452 | RASESVTILGSHLIY | Anti-BCMA C12A3.2 scFv |
| | | light chain CDR1 |
| 453 | LASNVQT | Anti-BCMA C12A3.2 scFv |
| | | light chain CDR2 |
| 454 | LQSRTIPRT | Anti-BCMA C12A3.2 scFv |
| | | light chain CDR3 |
| 455 | QIQLVQSGPELKKPGETVKISCKASGYTFRHYSMNWV | Anti-BCMA C12A3.2 scFv |
| | KQAPGKGLKWMGRINTESGVPIYADDFKGRFAFSVET | heavy chain variable region |
| | SASTAYLVINNLKDEDTASYFCSNDYLYSLDFWGQGT | |
| | ALTVSS | |
| 456 | HYSMN | Anti-BCMA C12A3.2 scFv |
| | | heavy chain CDR1 |
| 457 | RINTESGVPIYADDFKG | Anti-BCMA C12A3.2 scFv |
| | | heavy chain CDR2 |
| 458 | DYLYSLDF | Anti-BCMA C12A3.2 scFv |
| | | heavy chain CDR3 |
| 459 | EVQLLESGGGLVQPGGSLRLSCAASGFTFSSYAMSWV | Anti-BCMA FHVH33 entire |
| | RQAPGKGLEWVSSISGSGDYIYYADSVKGRFTISRDIS | sequence |
| | KNTLYLQMNSLRAEDTAVYYCAKEGTGANSSLADYR | |
| | GQGTLVTVSS | |
| 460 | GFTFSSYA | Anti-BCMA FHVH33 |
| | | CDR1 |
| 461 | ISGSGDYI | Anti-BCMA FHVH33 |
| | | CDR2 |
| 462 | AKEGTGANSSLADY | Anti-BCMA FHVH33 |
| | | CDR3 |
| 463 | DIQMTQSPSSLSASVGDRVTITCRASQSISSYLNWYQQ | Anti-BCMA CT103A scFv |
| | KPGKAPKLLIYAASSLQSGVPSRFSGSGSGTDFTLTISS | entire sequence, with |
| | LQPEDFATYYCQQKYDLLTFGGGTKVEIKGSTSGSGK | Whitlow linker |
| | PGSGEGSTKGQLQLQESGPGLVKPSETLSLTCTVSGGS | |
| | ISSSSYYWGWIRQPPGKGLEWIGSISYSGSTYYNPSLKS | |
| | RVTISVDTSKNQFSLKLSSVTAADTAVYYCARDRGDTI | |
| | LDVWGQGTMVTVSS | |
| 464 | DIQMTQSPSSLSASVGDRVTITCRASQSISSYLNWYQQ | Anti-BCMA CT103A scFv |
| | KPGKAPKLLIYAASSLQSGVPSRFSGSGSGTDFTLTISS | light chain variable region |
| | LQPEDFATYYCQQKYDLLTFGGGTKVEIK | |
| 465 | QSISSY | Anti-BCMA CT103A scFv |
| | | light chain CDR1 |
| 466 | AAS | Anti-BCMA CT103A scFv |
| | | light chain CDR2 |
| 467 | QQKYDLLT | Anti-BCMA CT103A scFv |
| | | light chain CDR3 |
| 468 | QLQLQESGPGLVKPSETLSLTCTVSGGSISSSSYYWGW | Anti-BCMA CT103A scFv |
| | IRQPPGKGLEWIGSISYSGSTYYNPSLKSRVTISVDTSK | heavy chain variable region |
| | NQFSLKLSSVTAADTAVYYCARDRGDTILDVWGQGT | |
| | MVTVSS | |
| 469 | GGSISSSSYY | Anti-BCMA CT103A scFv |
| | | heavy chain CDR1 |
| 470 | ISYSGST | Anti-BCMA CT103A scFv |
| | | heavy chain CDR2 |
| 471 | ARDRGDTILDV | Anti-BCMA CT103A scFv |
| | | heavy chain CDR3 |
| 472 | atggccttaccagtgaccgccttgctcctgccgctggccttgctg | Exemplary BCMA CAR |
| | ctccacgccgccaggccggacatccagatgacccagtctccatcc | nucleotide sequence |
| | tccctgtctgcatctgtaggagacagagtcaccatcacttgccgg | |
| | gcaagtcagagcattagcagctatttaaattggtatcagcagaaa | |
| | ccagggaaagcccctaagctcctgatctatgctgcatccagtttg | |
| | caaagtggggtcccatcaaggttcagtggcagtggatctgggaca | |
| | gatttcactctcaccatcagcagtctgcaacctgaagattttgca | |
| | acttactactgtcagcaaaaatacgacctcctcacttttggcgga | |
| | gggaccaaggttgagatcaaaggcagcaccagcggctccggcaag | |
| | cctggctctggcgagggcagcacaaagggacagctgcagctgcag | |
| | gagtcgggcccaggactggtgaagccttcggagaccctgtccctc | |
| | acctgcactgtctctggtggctccatcagcagtagtagttactac | |
| | tggggctggatccgccagcccccagggaaggggctggagtggatt | |
| | gggagtatctcctatagtgggagcacctactacaacccgtccctc | |
| | aagagtcgagtcaccatatccgtagacacgtccaagaaccagttc | |
| | tccctgaagctgagttctgtgaccgccgcagacacggcggtgtac | |
| | tactgcgccagagatcgtggagacaccatactagacgtatggggt | |
| | cagggtacaatggtcaccgtcagctcattcgtgcccgtgttcctg | |
| | cccgccaaacctaccaccacccctgcccctagacctcccacccca | |
| | gccccaacaatcgccagccagcctctgtctctgcggcccgaagcc | |
| | tgtagacctgctgccggcggagccgtgcacaccagaggcctggac | |
| | ttcgcctgcgacatctacatctgggcccctctggccggcacctgt | |
| | ggcgtgctgctgctgagcctggtgatcaccctgtactgcaaccac | |
| | cggaacaaacggggcagaaagaaactcctgtatatattcaaacaa | |
| | ccatttatgagaccagtacaaactactcaagaggaagatggctgt | |
| | agctgccgatttccagaagaagaagaaggaggatgtgaactgaga | |
| | gtgaagttcagcagatccgccgacgcccctgcctaccagcaggga | |
| | cagaaccagctgtacaacgagctgaacctgggcagacgggaagag | |
| | tacgacgtgctggacaagcggagaggccgggaccccgagatgggc | |
| | ggaaagcccagacggaagaacccccaggaaggcctgtataacgaa | |
| | ctgcagaaagacaagatggccgaggcctacagcgagatcggcatg | |
| | aagggcgagcggaggcgcggcaagggccacgatggcctgtaccag | |
| | ggcctgagcaccgccaccaaggacacctacgacgccctgcacatg | |
| | caggccctgccccccaga | |
| 473 | MALPVTALLLPLALLLHAARPDIQMTQSPSSLSASVGD | Exemplary BCMA CAR |
| | RVTITCRASQSISSYLNWYQQKPGKAPKLLIYAASSLQ | amino acid sequence |
| | SGVPSRFSGSGSGTDFTLTISSLQPEDFATYYCQQKYD | |
| | LLTFGGGTKVEIKGSTSGSGKPGSGEGSTKGQLQLQES | |
| | GPGLVKPSETLSLTCTVSGGSISSSSYYWGWIRQPPGK | |
| | GLEWIGSISYSGSTYYNPSLKSRVTISVDTSKNQFSLKL | |
| | SSVTAADTAVYYCARDRGDTILDVWGQGTMVTVSSF | |
| | VPVFLPAKPTTTPAPRPPTPAPTIASQPLSLRPEACRPA | |
| | AGGAVHTRGLDFACDIYIWAPLAGTCGVLLLSLVITL | |
| | YCNHRNKRGRKKLLYIFKQPFMRPVQTTQEEDGCSCR | |
| | FPEEEEGGCELRVKFSRSADAPAYQQGQNQLYNELNL | |
| | GRREEYDVLDKRRGRDPEMGGKPRRKNPQEGLYNEL | |
| | QKDKMAEAYSEIGMKGERRRGKGHDGLYQGLSTATK | |
| | DTYDALHMQALPPR | |
| 474 | RYTMH | CD3 CDR-H1 |
| 475 | YINPSRGYTNYNQKFKD | CD3 CDR-H2 |
| 476 | YYDDHYCLDY | CD3 CDR-H3 |
| 477 | RASSSVSYMN | CD3 CDR-L1 |
| 478 | DTSKVAS | CD3 CDR-L2 |
| 479 | QQWSSNPLT | CD3 CDR-L3 |
| 480 | DIKLQQSGAELARPGASVKMSCKTSGYTFTRYTMHW | CD3 VH |
| | VKQRPGQGLEWIGYINPSRGYTNYNQKFKDKATLTTD | |
| | KSSSTAYMQLSSLTSEDSAVYYCARYYDDHYCLDYW | |
| | GQGTTLTVSSVE | |
| 481 | DIQLTQSPAIMSASPGEKVTMTCRASSSVSYMNWYQQ | CD3 VL |
| | KSGTSPKRWIYDTSKVASGVPYRFSGSGSGTSYSLTISS | |
| | MEAEDAATYYCQQWSSNPLTFGAGTKLELK | |
| 482 | DIKLQQSGAELARPGASVKMSCKTSGYTFTRYTMHW | CD3 scFv |
| | VKQRPGQGLEWIGYINPSRGYTNYNQKFKDKATLTTD | |
| | KSSSTAYMQLSSLTSEDSAVYYCARYYDDHYCLDYW | |
| | GQGTTLTVSSVEGGSGGSGGSGGSGGVDDIQLTQSPAI | |
| | MSASPGEKVTMTCRASSSVSYMNWYQQKSGTSPKRW | |
| | IYDTSKVASGVPYRFSGSGSGTSYSLTISSMEAEDAAT | |
| | YYCQQWSSNPLTFGAGTKLELK | |
| 483 | SYAIS | CDR-H1 |
| 484 | IIDPSDGNTNYAQNFQG | CDR-H2 |
| 485 | ERAAAGYYYYMDV | CDR-H3 |
| 486 | RASQSISSYLN | CDR-L1 |
| 487 | AASSLQS | CDR-L2 |
| 488 | QQSYSTPLT | CDR-L3 |
| 489 | QVQLVQSGAEVKKPGASVKVSCKASGGTFSSYAISWV | CD8 scFv |
| | RQAPGQGLEWMGIIDPSDGNTNYAQNFQGRVTMTRD | |
| | TSTSTVYMELSSLRSEDTAVYYCAKERAAAGYYYYM | |
| | DVWGQGTTVTVSSGGGGSGGGGSGGGGSDIQMTQSP | |
| | SSLSASVGDRVTITCRASQSISSYLNWYQQKPGKAPKL | |
| | LIYAASSLQSGVPSRFSGSGSGTDFTLTISSLQPEDFAT | |
| | YYCQQSYSTPLTFGGGTKVEIKR | |
| 490 | DYYIQ | CDR-H1 |
| 491 | WINPNSGGTSYAQKFQG | CDR-H2 |
| 492 | EGDYYYGMDA | CDR-H3 |
| 493 | RSSQSLLHSNGYNYLD | CDR-L1 |
| 494 | LGSNRAS | CDR-L2 |
| 495 | MQGLQTPHT | CDR-L3 |
| 496 | QVQLVQSGAEVKKPGASVKVSCKASGYTFTDYYIQW | CD8 scFv |
| | VRQAPGQGLEWMGWINPNSGGTSYAQKFQGRVTMT | |
| | RDTSTSTVYMELSSLRSEDTAVYYCAKEGDYYYGMD | |
| | AWGQGTMVTVSSGGGGSGGGGSGGGGSDIVMTQSPL | |
| | SLPVTPGEPASISCRSSQSLLHSNGYNYLDWYLQKPGQ | |
| | SPQLLIYLGSNRASGVPDRFSGSGSGTDFTLKISRVEAE | |
| | DVGVYYCMQGLQTPHTFGQGTKVEIKR | |
| 497 | SYYMH | CDR-H1 |
| 498 | GFDPEDGETIYAQKFQG | CDR-H2 |
| 499 | DQGWGMDV | CDR-H3 |
| 500 | QQTYSTPYT | CDR-L3 |
| 501 | QVQLVQSGAEVKKPGASVKVSCKASGYTFTSYYMH | CD8 scFv |
| | WVRQAPGQGLEWMGGFDPEDGETIYAQKFQGRVTM | |
| | TRDTSTSTVYMELSSLRSEDTAVYYCARDQGWGMDV | |
| | WGQGTTVTVSSGGGGSGGGGSGGGGSDIQMTQSPSSL | |
| | SASVGDRVTITCRASQSISSYLNWYQQKPGKAPKLLIY | |
| | AASSLQSGVPSRFSGSGSGTDFTLTISSLQPEDFATYYC | |
| | QQTYSTPYTFGQGTKLEIKR | |
| 502 | NHYMH | CDR-H1 |
| 503 | WMNPNSGNTGYAQKFQG | CDR-H2 |
| 504 | SESGSDLDY | CDR-H3 |
| 505 | RASQTIGNYVN | CDR-L1 |
| 506 | GASNLHT | CDR-L2 |
| 507 | QQTYSAPLT | CDR-L3 |
| 508 | QVQLVQSGAEVKKPGASVKVSCKASGYTFTNHYMH | CD8 scFv |
| | WVRQAPGQGLEWMGWMNPNSGNTGYAQKFQGRVT | |
| | MTRDTSTSTVYMELSSLRSEDTAVYYCASSESGSDLD | |
| | YWGQGTLVTVSSGGGGSGGGGSGGGGSDIQMTQSPS | |
| | SLSASVGDRVTITCRASQTIGNYVNWYQQKPGKAPKL | |
| | LIYGASNLHTGVPSRFSGSGSGTDFTLTISSLQPEDFAT | |
| | YYCQQTYSAPLTFGGGTKVEIKR | |
| 509 | GYVMG | CDR-H1 |
| 510 | AISRGGLSTSYADSVKG | CDR-H2 |
| 511 | DRSDLYEITAASNIDS | CDR-H3 |
| 512 | QVQLVESGGGLVQAGGSLRLSCAASGRTNSNYAMG | CD117_B1 VHH |
| | WFRQAPGKEREFVSVIGRGLGSTYYADSVKGRFTISR | |
| | DNAKNTVYLQMNSLKPEDAAVYYCAADLEFVFDEM | |
| | GWIPGEHAYWGRGTLVTVSS | |
| 513 | QVQLVESGGRLVQAGESLRLSCSASGGTFSSYLMAWF | CD117_B2 VHH |
| | RQVPGKEREIVAFITWHGHTTYYTDSVKGRFAISRDN | |
| | ARNTMYLQMNSLRPEDTAVYYCAADQRQEFSGTYSR | |
| | EYNYWGQGTQVTVSS | |
| 514 | QVQLVESGGGLVQPGGSLRLSCAASGQTFTDYHHHV | CD117_B3 VHH |
| | AWFRQAPGRERDFVAVISWSTGNTDYADSQRARFTIS | |
| | KDNAKNTVYLQMNNLKFEDTAVYYCAARDYHPRES | |
| | YSADDYNYWGQGTLVTVSS | |
| 515 | EVQLVESGGGLVQAGGSLRLSCAASGSSINEMGYYRQ | CD117_B4 VHH |
| | APGKERAPRELVATITSGGSTNYADSVKGRFTISRDNA | |
| | KNTVYLQMNSLKPEDTAVYYCAALRIMTMGRPYDY | |
| | WGQGTQVTVSS | |
| 516 | EIQLQQSGPDLMKPGASVKISCKASGYSFTNYYVHWV | CD133 scFv (b3) |
| | KQSLDKSLEWIGYVDPFNGDFNYNQKFKDKATLTVD | |
| | KSSSTAYMHLSSLTSEDSAVYYCARGGLDWYDTSYW | |
| | YFDVWGAGTAVTVSSGGGGSGGGGSGGGGSNIVMTQ | |
| | SPKSMSMSLGERVTLSCKASENVDTYVSWYQQKPEQ | |
| | SPKVLIYGASNRYTGVPDRFTGSGSATDFSLTISNVQA | |
| | EDLADYHCGQSYRYPLTFGAGTKLELK | |
| 517 | EIQLQQSGPDLMKPGASVKISCKASGYSFTNYYVHWV | CD133 VH (b3) |
| | KQSLDKSLEWIGYVDPFNGDFNYNQKFKDKATLTVD | |
| | KSSSTAYMHLSSLTSEDSAVYYCARGGLDWYDTSYW | |
| | YFDVWGAGTAVTVSS | |
| 518 | NYYVH | CD133 CDR-H1 (b3) |
| | | (Kabat) |
| 519 | YVDPFNGDFNYNQKFKD | CD133 CDR-H2 (b3) |
| | | (Kabat) |
| 520 | GGLDWYDTSYWYFDV | CD133 CDR-H3 (b3) |
| | | (Kabat, Chothia) |
| 521 | NIVMTQSPKSMSMSLGERVTLSCKASENVDTYVSWY | CD133 VL (b3) |
| | QQKPEQSPKVLIYGASNRYTGVPDRFTGSGSATDFSLT | |
| | ISNVQAEDLADYHCGQSYRYPLTFGAGTKLELK | |
| 522 | KASENVDTYVS | CD133 CDR-L1 (b3) |
| | | (Kabat, Chothia) |
| 523 | GASNRYT | CD133 CDR-L2 (b3) |
| | | (Kabat) |
| 524 | GQSYRYPLT | CD133 CDR-L3 (b3) |
| | | (Kabat) |
| 525 | EVQLVESGGGLVQPGGSLRLSCAASGFNIYYYGSMH | CD133 scFv (b4) |
| | WVRQAPGKGLEWVASISPYYGSTYYADSVKGRFTISA | |
| | DTSKNTAYLQMNSLRAEDTAVYYCARHASSGYGHYA | |
| | VYGIDYWGQGTLVTVSSGGGGSGGGGSGGGGSDIQM | |
| | TQSPSSLSASVGDRVTITCRASQGSSYVAWYQQKPGK | |
| | APKLLIYSASYLYSGVPSRFSGSRSGTDFTLTISSLQPED | |
| | FATYYCQQGVWSLITFGQGTKVEIK | |
| 526 | EVQLVESGGGLVQPGGSLRLSCAASGFNIYYYGSMH | CD133 VH (b4) |
| | WVRQAPGKGLEWVASISPYYGSTYYADSVKGRFTISA | |
| | DTSKNTAYLQMNSLRAEDTAVYYCARHASSGYGHYA | |
| | VYGIDYWGQGTLVTVSS | |
| 527 | YYGSMH | CD133 CDR-H1 (b4) |
| | | (Kabat) |
| 528 | SISPYYGSTYYADSVKG | CD133 CDR-H2 (b4) |
| | | (Kabat) |
| 529 | HASSGYGHYAVYGIDY | CD133 CDR-H3 (b4) |
| | | (Kabat, Chothia) |
| 530 | DIQMTQSPSSLSASVGDRVTITCRASQGSSYVAWYQQ | CD133 VL (b4) |
| | KPGKAPKLLIYSASYLYSGVPSRFSGSRSGTDFTLTISS | |
| | LQPEDFATYYCQQGVWSLITFGQGTKVEIK | |
| 531 | RASQGSSYVA | CD133 CDR-L1 (b4) |
| | | (Kabat, Chothia) |
| 532 | SASYLYS | CD133 CDR-L1 (b4) |
| | | (Kabat, Chothia) |
| 533 | QQGVWSLIT | CD133 CDR-L1 (b4) |
| | | (Kabat, Chothia) |
| 534 | EVKLVESGPELVKPGETVKISCKASGYTFTDYSMHWV | CD133 scFv (b1) |
| | RQAPGKGLEWMGWINTETGEPSYADDFKGRFAFSLD | |
| | TSASTAYLQINSLRSEDTATYFCATDYGDYFDYWGQG | |
| | TTVTVSSGGGGSGGGGSGGGGSDIVLSQSPAIMSASPG | |
| | EKVTISCSASSSVSYMYWYQQKPGSSPKPWIYRTSNL | |
| | ASGVPARFSGSGSGTSYSLTISSMEPEDAATYYCQQYH | |
| | SYPPTFGAGTKLELK | |
| 535 | EVKLVESGPELVKPGETVKISCKASGYTFTDYSMHWV | CD133 VH (b1) |
| | RQAPGKGLEWMGWINTETGEPSYADDFKGRFAFSLD | |
| | TSASTAYLQINSLRSEDTATYFCATDYGDYFDYWGQG | |
| | TTVTVSS | |
| 536 | DYSMH | CD133 CDR-H1 (b1) |
| | | (Kabat) |
| 537 | WINTETGEPSYADDFKG | CD133 CDR-H2 (b1) |
| | | (Kabat) |
| 538 | DYGDYFDY | CD133 CDR-H3 (b1) |
| | | (Kabat, Chothia) |
| 539 | DIVLSQSPAIMSASPGEKVTISCSASSSVSYMYWYQQK | CD133 VL (b1) |
| | PGSSPKPWIYRTSNLASGVPARFSGSGSGTSYSLTISSM | |
| | EPEDAATYYCQQYHSYPPTFGAGTKLELK | |
| 540 | SASSSVSYMY | CD133 CDR-L1 (b1) |
| | | (Kabat, Chothia) |
| 541 | RTSNLAS | CD133 CDR-L2 (b1) |
| | | (Kabat, Chothia) |
| 542 | QQYHSYPPT | CD133 CDR-L3 (b1) |
| | | (Kabat, Chothia) |
| 543 | DVQLQESGPGLVKPSQSLSLTCSVTGYSITSGYYWSWI | CD133 scFv (b2) |
| | RQFPGNKLEWMGYISYDGSNNYNPSLKNRISITRDTSK | |
| | NQIFLKLNSVTTEDTATYYCTRVYFGHPYYYGMDYW | |
| | GQGISVTVGGGGSGGGGSGGGGSQIVLTQSPAIMSASL | |
| | GERVTMTCTASSSVSSFYLHWYRQKPGSSPKLWIYST | |
| | SNLASGVPARFSGSGSGTSYSLTISSMEAEDAATYYCH | |
| | QYHRSPYMYMFGGGTKLEIK | |
| 544 | DVQLQESGPGLVKPSQSLSLTCSVTGYSITSGYYWSWI | CD133 VH (b2) |
| | RQFPGNKLEWMGYISYDGSNNYNPSLKNRISITRDTSK | |
| | NQIFLKLNSVTTEDTATYYCTRVYFGHPYYYGMDYW | |
| | GQGISVTV | |
| 545 | SGYYWS | CD133 CDR-H1 (b2) |
| | | (Kabat) |
| 546 | YISYDGSNNYNPSLKN | CD133 CDR-H2 (b2) |
| | | (Kabat) |
| 547 | VYFQHPYYYGMDY | CD133 CDR-H3 (b2) |
| | | (Kabat, Chothia) |
| 548 | QIVLTQSPAIMSASLGERVTMTCTASSSVSSFYLHWYR | CD133 VL (b2) |
| | QKPGSSPKLWIYSTSNLASGVPARESGSGSGTSYSLTIS | |
| | SMEAEDAATYYCHQYHRSPYMYMFGGGTKLEIK | |
| 549 | TASSSVSSFYLH | CD133 CDR-L1 (b2) |
| | | (Kabat, Chothia) |
| 550 | STSNLAS | CD133 CDR-L2 (b2) |
| | | (Kabat, Chothia) |
| 551 | HQYHRSPYMYM | CD133 CDR-L3 (b2) |
| | | (Kabat, Chothia) |
| 552 | EVQLVESQGGLVQPGGSLRLSCAASGENLSSSSIHWVR | CD133 scFv (b5) |
| | QAPGKGLEWVAYTYPYYSYTYYADSVKGRFTISADTS | |
| | KNTAYLQMNSLRAEDTAVYYCARFGSVAGEDYWGQ | |
| | GILVTVSSGGGGSGGGGSGGGGSDIQMTQSPSSLSAS | |
| | VGDRVTITCRASQSVSSAVAWYQQKPGKAPKLLIYSA | |
| | SSLYSGVPSRFSGSRSGTDFTLTISSLQPEDFATYYCQQ | |
| | YSHAGHLFTFGQGTKVEIK | |
| 553 | EVQLVESGGGLVQPGGSLRLSCAASGENLSSSSIHWVR | CD133 VH (b5) |
| | QAPGKGLEWVAYTYPYYSYTYYADSVKGRFTISADTS | |
| | KNTAYLQMNSLRAEDTAVYYCARFGSVAGFDYWGQ | |
| | GTLVTVSS | |
| 554 | SSSIH | CD133 CDR-H1 (b5) |
| | | (Kabat) |
| 555 | YTYPYYSYTYYADSVKG | CD133 CDR-H2 (b5) |
| | | (Kabat) |
| 556 | FGSVAGEDY | CD133 CDR-H3 (b5) |
| | | (Kabat, Chothia) |
| 557 | DIQMTQSPSSLSASVGDRVTITCRASQSVSSAVAWYQ | CD133 VL (b5) |
| | QKPGKAPKLLIYSASSLYSGVPSRFSGSRSGTDFTLTIS | |
| | SLQPEDFATYYCQQYSHAGHLFTFGQGTKVEIK | |
| 558 | RASQSVSSAVA | CD133 CDR-L1 (b5) |
| | | (Kabat, Chothia) |
| 559 | SASSLYS | CD133 CDR-L2 (b5) |
| | | (Kabat, Chothia) |
| 560 | QQYSHAGHLFT | CD133 CDR-L3 (b5) |
| | | (Kabat, Chothia) |
| 561 | MMADSKLVSLNNNLSGKIKDQGKVIKNYYGTMDIKK | Henipavirus protein G |
| | INDGLLDSKILGAFNTVIALLGSIIIIVMNIMIIQNYTRTT | (GENBANK ID: AF017149) |
| | DNQALIKESLQSVQQQIKALTDKIGTEIGPKVSLIDTSS | |
| | TITIPANIGLLGSKISQSTSSINENVNDKCKFTLPPLKIHE | |
| | CNISCPNPLPFREYRPISQGVSDLVGLPNQICLQKTTSTI | |
| | LKPRLISYTLPINTREGVCITDPLLAVDNGFFAYSHLEK | |
| | IGSCTRGIAKQRIIGVGEVLDRGDKVPSMFMTNVWTPP | |
| | NPSTIHHCSSTYHEDFYYTLCAVSHVGDPILNSTSWTE | |
| | SLSLIRLAVRPKSDSGDYNQKYIAITKVERGKYDKVM | |
| | PYGPSGIKQGDTLYFPAVGFLPRTEFQYNDSNCPIIHCK | |
| | YSKAENCRLSMGVNSKSHYILRSGLLKYNLSLGGDIIL | |
| | QFIEIADNRLTIGSPSKIYNSLGQPVFYQASYSWDTMIK | |
| | LGDVDTVDPLRVQWRNNSVISRPGQSQCPRFNVCPEV | |
| | CWEGTYNDAFLIDRLNWVSAGVYLNSNQTAENPVFA | |
| | VFKDNEILYQVPLAEDDTNAQKTITDCFLLENVIWCIS | |
| | LVEIYDTGDSVIRPKLFAVKIPAQCSES | |
| 562 | MLSQLQKNYLDNSNQQGDKMNNPDKKLSVNFNPLEL | Henipavirus protein G |
| | DKGQKDLNKSYYVKNKNYNVSNLLNESLHDIKFCIYC | (GENBANK ID: JQ001776) |
| | IFSLLIIITIINIITISIVITRLKVHEENNGMESPNLQSIQDS | |
| | LSSLTNMINTEITPRIGILVTATSVTLSSSINYVGTKTNQ | |
| | LVNELKDYITKSCGFKVPELKLHECNISCADPKISKSA | |
| | MYSTNAYAELAGPPKIFCKSVSKDPDFRLKQIDYVIPV | |
| | QQDRSICMNNPLLDISDGFFTYIHYEGINSCKKSDSFKV | |
| | LLSHGEIVDRGDYRPSLYLLSSHYHPYSMQVINCVPVT | |
| | CNQSSFVFCHISNNTKTLDNSDYSSDEYYITYFNGIDRP | |
| | KTKKIPINNMTADNRYIHFTFSGGGGVCLGEEFIIPVTT | |
| | VINTDVFTHDYCESFNCSVQTGKSLKEICSESLRSPTNS | |
| | SRYNLNGIMIISQNNMTDFKIQLNGITYNKLSFGSPGRL | |
| | SKTLGQVLYYQSSMSWDTYLKAGFVEKWKPFTPNW | |
| | MNNTVISRPNQGNCPRYHKCPEICYGGTYNDIAPLDL | |
| | GKDMYVSVILDSDQLAENPEITVENSTTILYKERVSKD | |
| | ELNTRSTTTSCFLFLDEPWCISVLETNRFNGKSIRPEIYS | |
| | YKIPKYC | |
| 563 | MPQKTVEFINMNSPLERGVSTLSDKKTLNQSKITKQG | Henipavirus protein G |
| | YFGLGSHSERNWKKQKNQNDHYMTVSTMILEILVVL | (GENBANK ID: |
| | GIMFNLIVLTMVYYQNDNINQRMAELTSNITVLNLNL | NC_025256) |
| | NQLTNKIQREIIPRITLIDTATTITIPSAITYILATLTTRISE | |
| | LLPSINQKCEFKTPTLVLNDCRINCTPPLNPSDGVKMSS | |
| | LATNLVAHGPSPCRNFSSVPTIYYYRIPGLYNRTALDE | |
| | RCILNPRLTISSTKFAYVHSEYDKNCTRGFKYYELMTF | |
| | GEILEGPEKEPRMFSRSFYSPTNAVNYHSCTPIVTVNE | |
| | GYFLCLECTSSDPLYKANLSNSTFHLVILRHNKDEKIV | |
| | SMPSFNLSTDQEYVQIIPAEGGGTAESGNLYFPCIGRLL | |
| | HKRVTHPLCKKSNCSRTDDESCLKSYYNQGSPQHQV | |
| | VNCLIRIRNAQRDNPTWDVITVDLTNTYPGSRSRIFGSF | |
| | SKPMLYQSSVSWHTLLQVAEITDLDKYQLDWLDTPYI | |
| | SRPGGSECPFGNYCPTVCWEGTYNDVYSLTPNNDLFV | |
| | TVYLKSEQVAENPYFAIFSRDQILKEFPLDAWISSARTT | |
| | TISCFMFNNEIWCIAALEITRLNDDIIRPIYYSFWLPTDC | |
| | RTPYPHTGKMTRVPLRSTYNY | |
| 564 | MATNRDNTITSAEVSQEDKVKKYYGVETAEKVADSIS | Henipavirus protein G |
| | GNKVFILMNTLLILTGAIITITLNITNLTAAKSQQNMLK | (GENBANK ID: |
| | IIQDDVNAKLEMFVNLDQLVKGEIKPKVSLINTAVSVS | NC_025352) |
| | IPGQISNLQTKFLQKYVYLEESITKQCTCNPLSGIFPTSG | |
| | PTYPPTDKPDDDTTDDDKVDTTIKPIEYPKPDGCNRTG | |
| | DHFTMEPGANFYTVPNLGPASSNSDECYTNPSFSIGSSI | |
| | YMFSQEIRKTDCTAGEILSIQIVLGRIVDKGQQGPQASP | |
| | LLVWAVPNPKIINSCAVAAGDEMGWVLCSVTLTAAS | |
| | GEPIPHMFDGFWLYKLEPDTEVVSYRITGYAYLLDKQ | |
| | YDSVFIGKGGGIQKGNDLYFQMYGLSRNRQSFKALCE | |
| | HGSCLGTGGGGYQVLCDRAVMSFGSEESLITNAYLKV | |
| | NDLASGKPVIIGQTFPPSDSYKGSNGRMYTIGDKYGLY | |
| | LAPSSWNRYLRFGITPDISVRSTTWLKSQDPIMKILSTC | |
| | TNTDRDMCPEICNTRGYQDIFPLSEDSEYYTYIGITPNN | |
| | GGTKNFVAVRDSDGHIASIDILQNYYSITSATISCFMYK | |
| | DEIWCIAITEGKKQKDNPQRIYAHSYKIRQMCYNMKS | |
| | ATVTVGNAKNITIRRY | |
| 565 | NTETGE | CD133 CDR-H2 (b1) |
| | | (Chothia) |
| 566 | GYSITSGY | CD133 CDR-H1 (b2) |
| | | (Chothia) |
| 567 | SYDGS | CD133 CDR-H2 (b2) |
| | | (Chothia) |
| 568 | GYSFTNY | CD133 CDR-H1 (b3) |
| | | (Chothia) |
| 569 | DPFNGD | CD133 CDR-H2 (b3) |
| | | (Chothia) |
| 570 | GFNIYYYG | CD133 CDR-H1 (b4) |
| | | (Chothia) |
| 571 | SPYYGS | CD133 CDR-H2 (b4) |
| | | (Chothia) |
| 572 | GFNLSSS | CD133 CDR-H1 (b5) |
| | | (Chothia) |
| 573 | YPYYSY | CD133 CDR-H2 (b5) |
| | | (Chothia) |
| 574 | MKKINEGLLDSKILSAFNTVIALLGSIVIIVMNIMIIQNY | Blinded NiVG protein |
| | TRSTDNQAVIKDALQGIQQQIKGLADKIGTEIGPKVSLI | attachment glycoprotein + |
| | DTSSTITIPANIGLLGSKISQSTASINENVNEKCKFTLPP | linker + CD133 scFv (b1) |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIBISDQRLSIGSPSKIYDSLGQPVFY | |
| | QASFSWDTMIKFGDVLTVNPLVVNWRNNTVISRPGQS | |
| | QCPRENTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLPAVKIPEQCTGGG | |
| | GSGGGGSGGGGSEVKLVESGPELVKPGETVKISCKAS | |
| | GYTFTDYSMHWVRQAPGKGLEWMGWINTETGEPSY | |
| | ADDFKGRFAFSLDTSASTAYLQINSLRSEDTATYFCAT | |
| | DYGDYFDYWGQGTTVTVSSGGGGSGGGGSGGGGSDI | |
| | VLSQSPAIMSASPGEKVTISCSASSSVSYMYWYQQKPG | |
| | SSPKPWIYRTSNLASGVPARESGSGSGTSYSLTISSMEP | |
| | EDAATYYCQQYHSYPPTFGAGTKLELK | |
| 575 | MKKINEGLLDSKILSAFNTVIALLGSIVIIVMNIMIIQNY | CD117_A |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVELDSN | |
| | ATAANPVFTVFKDNEILYRAQLASEDTNAQKTIINCEL | |
| | LKNKIWCISLVEIYDTGDNVIRPKLFAVKIPEQCTGGG | |
| | GSGGGGSGGGGSQVQLVESGGRLVQAGESLRLSCSAS | |
| | GGTFSSYLMAWFRQVPGKEREIVAFITWHGHTTYYTD | |
| | SVKGRFAISRDNARNTMYLQMNSLRPEDTAVYYCAA | |
| | DQRQEFSGTYSREYNYWGQGTQVTVSSGGGGSGGGG | |
| | SGGGGSEVQLVESGGGLVQAGGSLRLSCAASGSSINE | |
| | MGYYRQAPGKERAPRELVATITSGGSTNYADSVKGRF | |
| | TISRDNAKNTVYLQMNSLKPEDTAVYYCAALRIMTM | |
| | GRPYDYWGQGTQVTVSS | |
| 576 | MKKINEGLLDSKILSAFNTVIALLGSIVIIVMNIMIIQNY | CD117_B |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCTGGG | |
| | GSGGGGSGGGGSQVQLVESGGRLVQAGESLRLSCSAS | |
| | GGTFSSYLMAWFRQVPGKEREIVAFITWHGHTTYYTD | |
| | SVKGRFAISRDNARNTMYLQMNSLRPEDTAVYYCAA | |
| | DQRQEFSGTYSREYNYWGQGTQVTVSSGGGGSGGGG | |
| | SGGGGSQVQLVESGGGLVQAGGSLRLSCAASGRTNS | |
| | NYAMGWFRQAPGKEREFVSVIGRGLGSTYYADSVKG | |
| | RFTISRDNAKNTVYLQMNSLKPEDAAVYYCAADLEF | |
| | VFDEMGWIPGEHAYWGRGTLVTVSS | |
| 577 | MKKINEGLLDSKILSAFNTVIALLGSIVIIVMNIMIIQNY | CD117_C |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCTGGG | |
| | GSGGGGSGGGGSEVQLVESGGGLVQAGGSLRLSCAA | |
| | SGSSINEMGYYRQAPGKERAPRELVATITSGGSTNYA | |
| | DSVKGRFTISRDNAKNTVYLQMNSLKPEDTAVYYCA | |
| | ALRIMTMGRPYDYWGQGTQVTVSSGGGGSGGGGSG | |
| | GGGS | |
| | QVQLVESGGGLVQAGGSLRLSCAASGRTNSNYAMG | |
| | WFRQAPGKEREFVSVIGRGLGSTYYADSVKGRFTISR | |
| | DNAKNTVYLQMNSLKPEDAAVYYCAADLEFVFDEM | |
| | GWIPGEHAYWGRGTLVTVSS | |
| 578 | MKKINEGLLDSKILSAFNTVIALLGSIVIIVMNIMIIQNY | CD117_D |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCTGGG | |
| | GSGGGGSGGGGSQVQLVESGGGLVQAGGSLRLSCAA | |
| | SGRTNSNYAMGWFRQAPGKEREFVSVIGRGLGSTYY | |
| | ADSVKGRFTISRDNAKNTVYLQMNSLKPEDAAVYYC | |
| | AADLEFVFDEMGWIPGEHAYWGRGTLVTVSSGGGGS | |
| | GGGGSGGGGSEVQLVESGGGLVQAGGSLRLSCAASG | |
| | SSINEMGYYRQAPGKERAPRELVATITSGGSTNYADS | |
| | VKGRFTISRDNAKNTVYLQMNSLKPEDTAVYYCAAL | |
| | RIMTMGRPYDYWGQGTQVTVSS | |
| 579 | MKKINEGLLDSKILSAFNTVIALLGSIVIIVMNIMIIQNY | CD117_E |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCTGGG | |
| | GSGGGGSGGGGSQVQLVESGGGLVQAGGSLRLSCAA | |
| | SGRTNSNYAMGWFRQAPGKEREFVSVIGRGLGSTYY | |
| | ADSVKGRFTISRDNAKNTVYLQMNSLKPEDAAVYYC | |
| | AADLEFVFDEMGWIPGEHAYWGRGTLVTVSSGGGGS | |
| | GGGGSGGGGSQVQLVESGGGLVQAGGSLRLSCAASG | |
| | RTNSNYAMGWFRQAPGKEREFVSVIGRGLGSTYYAD | |
| | SVKGRFTISRDNAKNTVYLQMNSLKPEDAAVYYCAA | |
| | DLEFVFDEMGWIPGEHAYWGRGTLVTVSS | |
| 580 | MKKINEGLLDSKILSAFNTVIALLGSIVIIVMNIMIIQNY | CD117_F |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCTGGG | |
| | GSGGGGSGGGGSEVQLVESGGGLVQAGGSLRLSCAA | |
| | SGSSINEMGYYRQAPGKERAPRELVATITSGGSTNYA | |
| | DSVKGRFTISRDNAKNTVYLQMNSLKPEDTAVYYCA | |
| | ALRIMTMGRPYDYWGQGTQVTVSSGGGGSGGGGSG | |
| | GGGS | |
| | EVQLVESGGGLVQAGGSLRLSCAASGSSINEMGYYRQ | |
| | APGKERAPRELVATITSGGSTNYADSVKGRFTISRDNA | |
| | KNTVYLQMNSLKPEDTAVYYCAALRIMTMGRPYDY | |
| | WGQGTQVTVSS | |
| 581 | MKKINEGLLDSKILSAFNTVIALLGSIVIIVMNIMIIQNY | CD117_E |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCTGGG | |
| | GSGGGGSGGGGSQVQLVESGGRLVQAGESLRLSCSAS | |
| | GGTFSSYLMAWFRQVPGKEREIVAFITWHGHTTYYTD | |
| | SVKGRFAISRDNARNTMYLQMNSLRPEDTAVYYCAA | |
| | DQRQEFSGTYSREYNYWGQGTQVTVSSGGGGSGGGG | |
| | SGGGGSQVQLVESGGRLVQAGESLRLSCSASGGTFSS | |
| | YLMAWFRQVPGKEREIVAFITWHGHTTYYTDSVKGR | |
| | FAISRDNARNTMYLQMNSLRPEDTAVYYCAADQRQE | |
| | FSGTYSREYNYWGQGTQVTVSS | |
| 582 | MKKINEGLLDSKILSAFNTVIALLGSIVIIVMNIMIIQNY | CD133_A |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCTGGG | |
| | GSGGGGSGGGGSEIQLQQSGPDLMKPGASVKISCKAS | |
| | GYSFTNYYVHWVKQSLDKSLEWIGYVDPFNGDFNYN | |
| | QKFKDKATLTVDKSSSTAYMHLSSLTSEDSAVYYCAR | |
| | GGLDWYDTSYWYFDVWGAGTAVTVSSGGGGSGGGG | |
| | SGGGGSNIVMTQSPKSMSMSLGERVTLSCKASENVDT | |
| | YVSWYQQKPEQSPKVLIYGASNRYTGVPDRFTGSGSA | |
| | TDFSLTISNVQAEDLADYHCGQSYRYPLTFGAGTKLE | |
| | LKGGGGSGGGGSGGGGSEVQLVESGGGLVQPGGSLR | |
| | LSCAASGFNIYYYGSMHWVRQAPGKGLEWVASISPY | |
| | YGSTYYADSVKGRFTISADTSKNTAYLQMNSLRAEDT | |
| | AVYYCARHASSGYGHYAVYGIDYWGQGTLVTVSSG | |
| | GGGSGGGGSGGGGSDIQMTQSPSSLSASVGDRVTITC | |
| | RASQGSSYVAWYQQKPGKAPKLLIYSASYLYSGVPSR | |
| | FSGSRSGTDFTLTISSLQPEDFATYYCQQGVWSLITFGQ | |
| | GTKVEIK | |
| 583 | MKKINEGLLDSKILSAFNTVIALLGSIVIIVMNIMIIQNY | CD133_B |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCTGGG | |
| | GSGGGGSGGGGSEVQLVESGGGLVQPGGSLRLSCAAS | |
| | GFNIYYYGSMHWVRQAPGKGLEWVASISPYYGSTYY | |
| | ADSVKGRFTISADTSKNTAYLQMNSLRAEDTAVYYC | |
| | ARHASSGYGHYAVYGIDYWGQGTLVTVSSGGGGSGG | |
| | GGSGGGGSDIQMTQSPSSLSASVGDRVTITCRASQGSS | |
| | YVAWYQQKPGKAPKLLIYSASYLYSGVPSRFSGSRSG | |
| | TDFTLTISSLQPEDFATYYCQQGVWSLITFGQGTKVEI | |
| | KGGGGSGGGGSGGGGSEIQLQQSGPDLMKPGASVKIS | |
| | CKASGYSFTNYYVHWVKQSLDKSLEWIGYVDPFNGD | |
| | FNYNQKFKDKATLTVDKSSSTAYMHLSSLTSEDSAVY | |
| | YCARGGLDWYDTSYWYFDVWGAGTAVTVSSGGGGS | |
| | GGGGSGGGGSNIVMTQSPKSMSMSLGERVTLSCKASE | |
| | NVDTYVSWYQQKPEQSPKVLIYGASNRYTGVPDRFT | |
| | GSGSATDFSLTISNVQAEDLADYHCGQSYRYPLTFGA | |
| | GTKLELK | |
| 584 | MKKINEGLLDSKILSAFNTVIALLGSIVIIVMNIMIIQNY | CD8_A |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | GSGGGGSGGGGSQVQLVESGGGLVQPGGSLRLSCAA | |
| | SGQTFTDYHHHVAWFRQAPGRERDFVAVISWSTGNT | |
| | DYADSQRARFTISKDNAKNTVYLQMNNLKFEDTAVY | |
| | YCAARDYHPRFSYSADDYNYWGQGTLVTVSSGGGGS | |
| | GGGGSGGGGSEVQLVESGGGLVQAGGSLRLSCAASG | |
| | SSINEMGYYRQAPGKERAPRELVATITSGGSTNYADS | |
| | VKGRFTISRDNAKNTVYLQMNSLKPEDTAVYYCAAL | |
| | RIMTMGRPYDYWGQGTQVTVSS | |
| 587 | MKKINEGLLDSKILSAFNTVIALLGSIVIIVMNIMIIQNY | CD117_I |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCTGGG | |
| | GSGGGGSGGGGSQVQLVESGGGLVQAGGSLRLSCAA | |
| | SGRTNSNYAMGWFRQAPGKEREFVSVIGRGLGSTYY | |
| | ADSVKGRFTISRDNAKNTVYLQMNSLKPEDAAVYYC | |
| | AADLEFVFDEMGWIPGEHAYWGRGTLVTVSS | |
| | GGGGSGGGGSGGGGSQVQLVESGGGLVQPGGSLRLS | |
| | CAASGQTFTDYHHHVAWFRQAPGRERDFVAVISWST | |
| | GNTDYADSQRARFTISKDNAKNTVYLQMNNLKFEDT | |
| | AVYYCAARDYHPRFSYSADDYNYWGQGTLVTVSS | |
| 588 | MKKINEGLLDSKILSAFNTVIALLGSIVIIVMNIMIIQNY | CD117_J |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCTGGG | |
| | GSGGGGSGGGGSQVQLVESGGRLVQAGESLRLSCSAS | |
| | GGTFSSYLMAWFRQVPGKEREIVAFITWHGHTTYYTD | |
| | SVKGRFAISRDNARNTMYLQMNSLRPEDTAVYYCAA | |
| | DQRQEFSGTYSREYNYWGQGTQVTVSSGGGGSGGGG | |
| | SGGGGSQVQLVESGGGLVQPGGSLRLSCAASGQTFTD | |
| | YHHHVAWFRQAPGRERDFVAVISWSTGNTDYADSQR | |
| | ARFTISKDNAKNTVYLQMNNLKFEDTAVYYCAARDY | |
| | HPRFSYSADDYNYWGQGTLVTVSSa | |
| 589 | AHHSED | IgG2c camelid hinge |
| 590 | EPKTPKPQPQPQPQPQPNPTTE | IgG2b camelid hinge |
| 591 | EPKIPQPQPKPQPQPQPQPKPQPKPEPECTCPKCP | IgG2b camelid hinge |
| 592 | TNEVCKCPKCP | IgG2b camelid hinge |
| 593 | EVQLVESGGGLVQAGGSLRLSCKASGRTNSNYAMGW | CD117_b1 |
| | FRQAPGKEREFVTVIGRSIGMTYYADSVKGRFTISRDN | |
| | AKNTVYLQMNSLKPEDTAVYYCAADLEFVFGETGWI | |
| | PGDDAYWGQGTQVTVSS | |
| 594 | QVQLVESGGGLVQAGGSLRLSCAASGRTNSNYAMG | CD117_b2 |
| | WFRQAPGKEREFVSVIGRGLGSTYYADSVKGRFTISR | |
| | DNAKNTVYLQVNSLKPEDTAVYYCAADLEFVFDEMG | |
| | WIPGEHAYWGRGTLVTVSS | |
| 595 | EVQLVESGGGLVQPGGSLRLSCTASGRTNSNYAMGW | CD117_b3 |
| | FRQAPGKEREFVSVIGRGLGSTYYADSVKGRFTISRDN | |
| | AKNTVYLQMNSLKPEDTAVYYCAADLEFVFDETSWIP | |
| | GEHAYWGRGTLVTVSS | |
| 596 | EVQLVESGGGLVQAGGSLRLSCAASGRTNSNYAMGW | CD117_b4 |
| | FRQAPGKEREFVTVIGRSIGMTYYADSVKGRFTISRDN | |
| | AKNTVYLQMNSLKPEDTAVYYCAADLEFVFDETGWI | |
| | PGDDAYWGQGTLVTVSS | |
| 597 | QVQLVESGGGLVQAGGSLRLSCAASGRTNSNYAMG | CD117_b5 |
| | WFRQAPGKEREFVSVIGRGLGSTYYADSVKGRFTISR | |
| | DNAKNTVYLQMNSLKPEDTAVYYCAADLEFVFDGM | |
| | GWIPGEHAYWGRGTLVTVSS | |
| 598 | EVQLVESGGGLVQAGGSLRLSCAASGRTNSNYAMGW | CD117_b6 |
| | FRQAPGKEREFVSVIGRGLGSTYYADSVKGRFTISRDN | |
| | AKNTVYLQMNSLKPEDTAVYYCAADLEFVFDEMGWI | |
| | PGEHAYWGRGTLVTVSS | |
| 599 | EVQLVESGGRLVRAGGSLRLSCAASGRTNSNYAMGW | CD117_b7 |
| | FRQAPGKEREFVSVIGRGLGSTYYADSVKGRFTISRDN | |
| | AKNTVYLQMNSLKPEDTAVYFCAADLEFVFDEMGWI | |
| | PGEHAYWGRGTLVTVSS | |
| 600 | EVQLVESGGGLVQAGGSLRLSCAASGRTNSNYAMGW | CD117_b8 |
| | FRQAPGKEREFVSVIGRGLGSTYYADSVKGRFTISRDN | |
| | AKNTVYLQMNSLKPEDTSVYYCAADLEFVFDEMGWI | |
| | PGEHAYWGRGTQVTVSS | |
| 601 | QVQLVESGGGLVQAGGSLGLSCAASGRTFSRMMMG | CD117_b9 |
| | WFRQAPGKEREFVSVIGRGLGSTYYADSVKGRFTISR | |
| | DNAKNTVYLQMNSLKPEDTAVYYCAADLEFVFDEM | |
| | GWIPGEHAYWGRGTQVTVSS | |
| 602 | QVQLVESGGGLVQAGGSLRLSCAASGRTNSNYAMG | CD117_b10 |
| | WFRQAPGKEREFVSVIGRGLGSTYYADSVKGRFTISR | |
| | DNAKNTVYLQMNSLKPEDTAVYYCAADLEFVFDEM | |
| | GWIPGEHAYWGRGTQVTVSS | |
| 603 | QVQLVESGGGLVQAGGSLRLSCAASGRTNSNYAMG | CD117_b11 |
| | WFRQAPGKEREFVSVIGRGLGSTYCADSVKGRFTTSR | |
| | DNAKNTVYLQMNSLKPEDTAVYYCAADLEFVFDEM | |
| | GWIPGEHAYWGRGTQVTVSS | |
| 604 | EVQLVESGGGLVQAGGSLRLSCAASGRTNSNYAMGW | CD117_b13 |
| | FRQAPGKEREFVSVIGRGLGSTYYADSVKGRFTISRDN | |
| | AKNTVYLQMNSLKPEDTAVYYCAADLEFVFDEMGWI | |
| | PGEHAYWGRGTQVTVSS | |
| 605 | EVQLVESGGGLVQAGGSLRLSCAASGRTNSDYAMGW | CD117_b14 |
| | FRQAPGKEREFVSVIGRGLGSTYYADSVKGRFTISRDN | |
| | AKNTVYLQMNSLKPEDTAVYYCAADLEFVFDEMGWI | |
| | PGEHAYWGRGTQVTVSS | |
| 606 | EVQLVESGGGLVQAGGSLRLSCAASGRTNSNYAMGW | CD117_b15 |
| | FRQAPGKEREFVSVIGRGLGSTYYADSVKGRFTISRDN | |
| | AKNTVYLQMNSLKPEDTAVYYCVADLEFVFDEMGWI | |
| | PGEHAYWGRGTLVTVSS | |
| 607 | EVQLVESGGGLAQAGDSLRLSCAASGRFDTSYAMGW | CD117_b16 |
| | FRQAPGKEREFVAGILSGTVKAYEDSVRGRFSISRDKA | |
| | KNTVYLQMNSLKSEDTAVYYCVADVSFGDLTNGRSD | |
| | YWGQGTQVTVSS | |
| 608 | EVQLVESGGGLAQAGDSLRLSCAASGRFDTSYAMGW | CD117_b17 |
| | FRQAPGKEREFVAGILSGTVKAYEDSVRGRFSISRDKA | |
| | KNTVYLQMNSLKPEDTAVYYCVADVSFGDLINGRSD | |
| | YWGQGTQVTVSS | |
| 609 | EVQLVESGGGLAQAGDSLRLSCAASGRFDTSYAMGW | CD117_b18 |
| | FRQAPGKEREFVAGILSGTVKAYEDSVRGRFSISRDKA | |
| | KNTVYLQMNSLKPEDTAVYYCVADVSFGDLINGRSD | |
| | YWGQGTLVTVSS | |
| 610 | QVQLVESGGGLAQAGDSLRLSCAASGREDTSYAMGW | CD117_b19 |
| | FRQAPGKEREFVAGILSGTVKAYEDSVRGRFSISRDKA | |
| | KNTVYLQMNSLKSEDTAVYYCVADVSFGDLTNGRSD | |
| | YWGQGTQVTVSS | |
| 611 | EVQLVESGGGLAQAGDSLRLSCAASGRFDTSYAMGW | CD117_b20 |
| | FRQAPGKEREFVAGILSGTVKAYEDSVRGRFSISRDKA | |
| | KNTVYLQMNSLKSEDTAVYYCVADVSFGDLINGRSD | |
| | YWGQGTLVTVSS | |
| 612 | EVQLVESGGGLAQAGDSLRLSCAASGRFDTSYAMGW | CD117_b21 |
| | FRQAPGKEREFVAGILSGAVKAYEDSVRGRFSISRDKA | |
| | KNTVYLQMNSLKPEDTAVYYCVADVSFGDLINGRSD | |
| | YWGQGTQVTVSS | |
| 613 | QVQLVESGGGLAQAGDSLRLSCAASGRFDTSYAMGW | CD117_b22 |
| | FRQAPGKEREFVAGILSGTVKAYEDSVRGRFSISRDKA | |
| | KNTVYLQMNSLKPEDTAVYYCVADVSFGDLINGRSD | |
| | YWGQGTQVTVSS | |
| 614 | EVQLVESGGGLAQAGDSLRLSCAASGRFDASYAMGW | CD117_b23 |
| | FRQAPGKEREFVAGILSGTVKAYEDSVRGRFSISRDKA | |
| | KNTVYLQMNSLKSEDTAVYYCVADVSFGDLINGRSD | |
| | YWGQGTLVTVSS | |
| 615 | EVQLVESGGGLAQAGDSLRLSCAASGRFDTSYAMGW | CD117_b24 |
| | FRQAPGKEREFVAGILSGTVKAYEDSVRGRFSISRDQA | |
| | KNTVYLQMNSLKPEDTAVYYCVADVSFGDLTNGRSD | |
| | YWGQGTQVTVSS | |
| 616 | EVQLVESGGGLAQAGDSLRLSCAASGRFDTSYAMGW | CD117_b25 |
| | FRQVPGKERGFVAGILSGTVKAYEDSVRGRFSISRDKA | |
| | KNTVYLRMNSLKPEDTAVYYCVADVSFGDLTNGRSD | |
| | YWGQGTQVTVSS | |
| 617 | EVQLVESGGGLVRAGDSLRLSCAASEGRTFTMRSVG | CD117_b26 |
| | WFRQAPGKEREFVAASNWWSSGSTMYAEYVKGRFTI | |
| | SRDNAKNTVYLQMNSLKPEDTATYYCAASSTVYMSS | |
| | RLSEYAYWGQGTQVTVSS | |
| 618 | QVQLVESGGGLVRAGDSLRLSCAASEGRTFTMRSVG | CD117_b27 |
| | WFRQAPGKEREFVAASNWWSSGSTMYAEYVKGRFTI | |
| | SRDNAKNTVYLQMNSLKPEDTATYYCAASSTVYMSS | |
| | RLSEYAYWGQGTQVTVSS | |
| 619 | EVQLVESGGGLVQTGGSLTLSCAASKRTLNTYAMGW | CD117_b28 |
| | FRQPPGKRREFVAADSWSGGSTMYAEYVKGRFTISRD | |
| | NAKNTVYLQMNSLKPEDTATYYCAASSTVYMSSRLS | |
| | EYAYWGQGTQVTVSS | |
| 620 | EVQLVESGGGWVQTDDSLRLSCAASGGAFGNYAMG | CD117_b29 |
| | WLRQAPGKGREFVAGIDWSGGSTMYAEYVKGRFTIS | |
| | RDNAKNTVYLQMNSLKPEDTATYYCAASSTVYMSSR | |
| | LSEYAYWGQGTLVTVSS | |
| 621 | QVQLVESGGGWVQTDDSLRLSCAASGGAFGNYAMG | CD117_b30 |
| | WLRQAPGKGREFVAGIDWSGGSTMYAEYVKGRFTIS | |
| | RDNAKNTVYLQMNSLKPEDTATYYCAASSTVYMSSR | |
| | LSEYAYWGQGTQVTVSS | |
| 622 | QVQLVESGGGSVQTGDSLRLSCVASGSTFGSYAMAW | CD117_b31 |
| | YRQVPGKEREFVAAVRWSGGSTMYAEYVKGRFTISR | |
| | DNAKNTVYLQMNGLKPEDTATYYCAASSTVYMSSRL | |
| | SEYAYWGQGTLVTVSS | |
| 623 | EVQLVESGGRLVQAGESLRLSCSASGGTFSSYLMAWF | CD117_b32 |
| | RQVPGKEREIVAFITWHGHTTYYTDSVKGRFAISRDN | |
| | ARNTMYLQMNSLRPEDTAVYYCAADQRQEFSGTYSR | |
| | EYNYWGQGTLVTVSS | |
| 624 | QVQLVESGGRLVQAGESLRLSCSASGGTFSSYLMAWF | CD117_b33 |
| | RQVPGKEREIVAFITWHGHTTYYTDSVKGRFAISRDN | |
| | ARNTMYLQMNSLRPEDTAVYYCAADQRQEFSGTYSR | |
| | EYNYWGQGTLVTVSS | |
| 625 | EVQLVESGGGLVQAGGSLGLACVASGDTLSTYFMGW | CD117_b35 |
| | FRQAPGKEREFVAAIGWSGWLTYYADSMKGRFTISRD | |
| | NGKNSVYLQMNNLKRDDTAVYYCAAGKTFAAAPST | |
| | TLIGDYWGQGTLVTVSS | |
| 626 | EVQLVESGGGLVQAGGSLRLACVASGDTLSTYFMGW | CD117_b36 |
| | FRQAPGKEREFVAAIGWSGWLTYYADSMKGRFTISRD | |
| | NGKNSVYLQMNNLKRDDTAVYYCAAGKTFAAAPST | |
| | TLIGDYWGQGTQVTVSS | |
| 627 | QVQLVESGGGLVQAGGSLRLSCVASGDTLSTYFMGW | CD117_b37 |
| | FRQAPGKEREFVAAIGWSGWLTYYADSMKGRFTISRD | |
| | NGKNSVYLQMNNLKRDDTAVYYCAAGKTFAAAPST | |
| | TLIGDYWGQGTLVTVSS | |
| 628 | EVQLVESGGGLVQVGGSLRLSCAASGRTTGRYFMGW | CD117_b38 |
| | FREAPGKEREFVSAIGWSVGSTYYVDSVKGRFTISRDN | |
| | AKNTVYLQMNSLKPEDTAVYYCAVGFEFSQGAGTAE | |
| | LIGDYWGQGTQVTVSS | |
| 629 | EVQLVESGGGLVQVGGSLRLSCAASGRTTGRYFMGW | CD117_b39 |
| | FREAPGKEREFVSAIGWSVGSTYYVDSVKGRFTISRDN | |
| | ANNTVYLQMNSLKPEDTAVYYCAVGFEFSQGAGTAE | |
| | LIGDYWGQGTQVTVSS | |
| 630 | QVQLVESGGGLVQAGGSLTLSCAAPGRTFSNYFMGW | CD117_b40 |
| | FRQAPGKERDFVAAISWSGFLTYYADSVKGRFTISRD | |
| | NAKNTVDLQMNRLKPEDTAVYYCAMGSEFSPGAGTA | |
| | ELIGDYWGQGTLVTVSS | |
| 631 | QVQLVESGGGLVQPGGSLRLSCAASGQTFTDYHHHV | CD117_b42 |
| | AWFRQAPGRERDFVAVISWSTGNTDYADSQRARFTIS | |
| | KDNAKNTAYLQMNNLKFEDTAVYYCAARDYHPRFS | |
| | YSADDYNYWGQGTQVTVSS | |
| 632 | EVQLVESGGGLVQAGDSLRLSCTASISTSGNYATFKN | CD117_b43 |
| | YAMGWFRQAPGKAREFVAGISNSGGTTDYADSVKGR | |
| | FTISRDNARKTTYLQMNRLGPEDTAVYSCAADAWFG | |
| | PPKRDPTTYSFRGPGTLVTVSS | |
| 633 | EVQLVESGGGLVQAGDSLRLSCAVSGRTFSSVAMAW | CD117_b44 |
| | LRQGPGKAREFVASISWSGSTTDYADSVKGRFTISRDN | |
| | AKKMVYLQMNSLKPEDTAVYYCAAGRVPGSSPTIHN | |
| | YVTWGQGTQVTVSS | |
| 634 | EVQLVESGGGSAHTGGPLRLSCVGTGPTTGHTFRGYA | CD117_b45 |
| | VAWFRQAPGKEREFVAAVRWNEGQDVYYADSVKGR | |
| | FTISRDNDKNTVYLQMNSLKPEDTAVYYCAAGVKWG | |
| | SITFMASSSRYDFWGQGTQVTVSS | |
| 635 | EVQLVESGGGLVQAGGSLRLSCAASGRTFSYYIMGWF | CD117_b47 |
| | RQAPGKEREFVAAINRSGGTTYYADSVEGRFFISRDNA | |
| | KNTMYLQMNSLKPEDTAVYYCAAQPDSRGTYGYWG | |
| | QGTLVTVSS | |
| 636 | EVQLVESGGGLVQAGGSLRLSCIDSGRTFSRYYMGWF | CD117_b48 |
| | RQAPGKEREFVAAISQNGYTKWYAESVKGRSAISRDI | |
| | AKNTVDLQMNSLKPEDTAVYYCAATSILEFSRDTASD | |
| | FEYYGQGTLVTVSS | |
| 637 | EVQLVESGGGLVRAGDSLRLSCSASGRTFSKWTMAW | CD117_b49 |
| | FRQAPGKEREFVAGMSGDATYYAASVKGRFTSSRDN | |
| | SKNTVYLQMDSLKPEDTAVYFCAIYYGSGSYFQQNSY | |
| | DTWGQGTQVTVSS | |
| 638 | QVQLVESGGGLVQAGGSLRLSCTASGRTESTYFMSWF | CD117_b50 |
| | RQTPGKERDFVAVINYSGDVTTYADSVKGRFTISRDN | |
| | AKNTVHLQMNSLTVEDTAVYYCAVHRTGPMNRADS | |
| | YDYWGQGTLVTVSS | |
| 639 | EVQLVESGGGLVQAGGSLKLSCAASGNIANTYRMAW | CD117_b51 |
| | YGQAPGKQRELVANITSDGIASYADSVKGRFTISRDNA | |
| | KNTVYLQMNSLKAEDTAVYYCNIVYRRCSRGGYGCY | |
| | SEYSWGQGTLVTVSS | |
| 640 | MKKINEGLLDSKILSAFNTVIALLGSIVIIVMNIMIIQQY | NivG.690 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 641 | MKKINEGLLDSKILSAFNTVIALLGSIVIIVMNIMIIQNY | NivG.691 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 642 | MKKINEGLLDSKILSAFNTVIALLGSIVIIVMNIMIIQNY | NivG.693 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 643 | MKKINEGLLDSKILSAFNTVIALLGSIVIIVMNIMIIQNY | NivG.694 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 644 | MKKINEGLLDSKILSAFNTVIALLGSIVIIVMNIMIIQNY | NivG.695 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 645 | MKKINEGLLDSKILSAFNTVIALLGSIVIIVMNIMIIQNY | NivG.696 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 646 | MKKINEGLLDSKILSAFNTVIALLGSIVIIVMNIMIIQNY | NivG.697 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 647 | MKKINEGLLDSKILSAFNTVIALLGSIVIIVMNIMIIQNY | NivG.699 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 648 | MKKINEGLLDSKILSAFNTVIALLGSIVIIVMNIMIIQNY | NivG.700 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 649 | MKKINEGLLDSKILSAFNTVIALLGSIVIIVMNIMIIQNY | NivG.701 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 650 | MKKINEGLLDSKILSAFNTVIALLGSIVIIVMNIMIIQQY | NivG.702 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 651 | MKKINEGLLDSKILSAFNTVIALLGSIVIIVMNIMIIQNY | NivG.740 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 652 | MKKINEGLLDSKILSAFNTVIALLGSIVIIVMNIMIIQNY | NivG.742 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 653 | MKKINEGLLDSKILSAFNTVIALLGSIVIIVMNIMIIQNY | NivG.743 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 654 | MKKINEGLLDSKILSAFNTVIALLGSIVIIVMNIMIIQNY | NivG.744 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 655 | MKKINEGLLDSKILSAFNTVIALLGSIVIIVMNIMIIQNY | NivG.748 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 656 | MKKINEGLLDSKILSAFNTVIALLGSIVIIVMNIMIIQNY | NivG.749 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 657 | MKKINEGLLDSKILSAFNTVIALLGSIVIIVMNIMIIQNY | NivG.750 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 658 | MKKINEGLLDSKILSAFNTVIALLGSIVIIVMNIMIIQNY | NivG.752 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 659 | MKKINEGLLDSKILSAFNTVIALLGSIVIIVMNIMIIQNY | NivG.753 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 660 | MKKINEGLLDSKILSAFNTVIALLGSIVIIVMNIMIIQNY | NivG.754 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 661 | MKKINEGLLDSKILSAFNTVIALLGSIVIIVMNIMIIQNY | NivG.755 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 662 | MKKINEGLLDSKILSAFNTVIALLGSIVIIVMNIMIIQNY | NivG.756 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 663 | MKKINEGLLDSKILSAFNTVIALLGSIVIIVMNIMIIQNY | NivG.757 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 664 | MKKINEGLLDSKILSAFNTVIALLGSIVIIVMNIMIIQNY | NivG.758 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 665 | MKKINEGLLDSKILSAFNTVIALLGSIVIIVMNIMIIQNY | NivG.759 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 666 | MKKINEGLLDSKILSAFNTVIALLGSIVIIVMNIMIIQNY | NivG.760 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 667 | MKKINEGLLDSKILSAFNTVIALLGSIVIIVMNIMIIQNY | NivG.761 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 668 | MKKINEGLLDSKILSAFNTVIALLGSIVIIVMNIMIIQNY | NivG.762 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 669 | MKKINEGLLDSKILSAFNTVIALLGSIVIIVMNIMIIQNY | NivG.763 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 670 | MKKINEGLLDSKILSAFNTVIALLGSIVIIVMNIMIIQNY | NivG.764 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 671 | MKKINEGLLDSKILSAFNTVIALLGSIVIIVMNIMIIQNY | NivG.765 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 672 | MKKINEGLLDSKILSAFNTVIALLGSIVIIVMNIMIIQNY | NivG.766 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 673 | MKKINEGLLDSKILSAFNTVIALLGSIVIIVMNIMIIQNY | NivG.767 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 674 | MKKINEGLLDSKILSAFNTVIALLGSIVIIVMNIMIIQNY | NivG.768 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 675 | MKKINEGLLDSKILSAFNTVIALLGSIVIIVMNIMIIQNY | NivG.769 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 676 | MKKINEGLLDSKILSAFNTVIALLGSIVIIVMNIMIIQNY | NivG.770 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 677 | MKKINEGLLDSKILSAFNTVIALLGSIVIIVMNIMIIQNY | NivG.771 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 678 | MKKINEGLLDSKILSAFNTVIALLGSIVIIVMNIMIIQNY | NivG.772 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 679 | MKKINEGLLDSKILSAFNTVIALLGSIVIIVMNIMIIQNY | NivG.773 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 680 | MKKINEGLLDSKILSAFNTVIALLGSIVIIVMNIMIIQNY | NivG.774 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 681 | MKKINEGLLDSKILSAFNTVIALLGSIVIIVMNIMIIQNY | NivG.775 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 682 | MKKINEGLLDSKILSAFNTVIALLGSIVIIVMNIMIIQNY | NivG.776 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 683 | MKKINEGLLDSKILSAFNTVIALLGSIVIIVMNIMIIQNY | NivG.777 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 684 | MKKINEGLLDSKILSAFNTVIALLGSIVIIVMNIMIIQNY | NivG.778 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 685 | MKKINEGLLDSKILSAFNTVIALLGSIVIIVMNIMIIQNY | NivG.779 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 686 | MKKINEGLLDSKILSAFNTVIALLGSIVIIVMNIMIIQNY | NivG.780 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 687 | MKKINEGLLDSKILSAFNTVIALLGSIVIIVMNIMIIQNY | NivG.781 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 688 | MKKINEGLLDSKILSAFNTVIALLGSIVIIVMNIMIIQNY | NivG.782 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 689 | MKKINEGLLDSKILSAFNTVIALLGSIVIIVMNIMIIQNY | NivG.783 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 690 | MKKINEGLLDSKILSAFNTVIALLGSIVIIVMNIMIIQNY | NivG.784 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 691 | MKKINEGLLDSKILSAFNTVIALLGSIVIIVMNIMIIQNY | NivG.785 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 692 | MKKINEGLLDSKILSAFNTVIALLGSIVIIVMNIMIIQNY | NivG.786 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 693 | MKKINEGLLDSKILSAFNTVIALLGSIVIIVMNIMIIQNY | NivG.787 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 694 | MKKINEGLLDSKILSAFNTVIALLGSIVIIVMNIMIIQNY | NivG.788 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 695 | MKKINEGLLDSKILSAFNTVIALLGSIVIIVMNIMIIQNY | NivG.789 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 696 | MKKINEGLLDSKILSAFNTVIALLGSIVIIVMNIMIIQNY | NivG.790 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 697 | MKKINEGLLDSKILSAFNTVIALLGSIVIIVMNIMIIQNY | NivG.791 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 698 | MKKINEGLLDSKILSAFNTVIALLGSIVIIVMNIMIIQNY | NivG.792 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 699 | MKKINEGLLDSKILSAFNTVIALLGSIVIIVMNIMIIQNY | NivG.793 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 700 | MKKINEGLLDSKILSAFNTVIALLGSIVIIVMNIMIIQNY | NivG.794 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 701 | MKKINEGLLDSKILSAFNTVIALLGSIVIIVMNIMIIQNY | NivG.795 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 702 | MKKINEGLLDSKILSAFNTVIALLGSIVIIVMNIMIIQNY | NivG.796 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 703 | MKKINEGLLDSKILSAFNTVIALLGSIVIIVMNIMIIQNY | NivG.797 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 704 | MKKINEGLLDSKILSAFNTVIALLGSIVIIVMNIMIIQNY | NivG.798 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 705 | MKKINEGLLDSKILSAFNTVIALLGSIVIIVMNIMIIQQY | NivG.799 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 706 | MKKINEGLLDSKILSAFNTVIALLGSIVIIVMNIMIIQQY | NivG.800 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 707 | MKKINEGLLDSKILSAFNTVIALLGSIVIIVMNIMIIQQY | NivG.801 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 708 | MKKINEGLLDSKILSAFNTVIALLGSIVIIVMNIMIIQQY | NivG.802 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 709 | MKKINEGLLDSKILSAFNTVIALLGSIVIIVMNIMIIQQY | NivG.803 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 710 | MKKINEGLLDSKILSAFNTVIALLGSIVIIVMNIMIIQQY | NivG.804 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 711 | MKKINEGLLDSKILSAFNTVIALLGSIVIIVMNIMIIQQY | NivG.805 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 712 | MKKINEGLLDSKILSAFNTVIALLGSIVIIVMNIMIIQQY | NivG.806 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 713 | MKKINEGLLDSKILSAFNTVIALLGSIVIIVMNIMIIQQY | NivG.807 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 714 | MKKINEGLLDSKILSAFNTVIALLGSIVIIVMNIMIIQQY | NivG.808 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 715 | MKKINEGLLDSKILSAFNTVIALLGSIVIIVMNIMIIQQY | NivG.809 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 716 | MKKINEGLLDSKILSAFNTVIALLGSIVIIVMNIMIIQQY | NivG.810 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 717 | MKKINEGLLDSKILSAFNTVIALLGSIVIIVMNIMIIQQY | NivG.811 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 718 | MKKINEGLLDSKILSAFNTVIALLGSIVIIVMNIMIIQQY | NivG.812 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 719 | MKKINEGLLDSKILSAFNTVIALLGSIVIIVMNIMIIQQY | NivG.813 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 720 | MKKINEGLLDSKILSAFNTVIALLGSIVIIVMNIMIIQQY | NivG.814 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 721 | MKKINEGLLDSKILSAFNTVIALLGSIVIIVMNIMIIQQY | NivG.815 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 722 | MKKINEGLLDSKILSAFNTVIALLGSIVIIVMNIMIIQQY | NivG.816 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 723 | MKKINEGLLDSKILSAFNTVIALLGSIVIIVMNIMIIQQY | NivG.817 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 724 | MKKINEGLLDSKILSAFNTVIALLGSIVIIVMNIMIIQQY | NivG.818 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 725 | MKKINEGLLDSKILSAFNTVIALLGSIVIIVMNIMIIQQY | NivG.819 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 726 | MKKINEGLLDSKILSAFNTVIALLGSIVIIVMNIMIIQQY | NivG.820 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 727 | MKKINEGLLDSKILSAFNTVIALLGSIVIIVMNIMIIQQY | NivG.821 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 728 | MKKINEGLLDSKILSAFNTVIALLGSIVIIVMNIMIIQQY | NivG.822 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 729 | MKKINEGLLDSKILSAFNTVIALLGSIVIIVMNIMIIQQY | NivG.823 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 730 | MKKINEGLLDSKILSAFNTVIALLGSIVIIVMNIMIIQQY | NivG.824 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 731 | MKKINEGLLDSKILSAFNTVIALLGSIVIIVMNIMIIQQY | NivG.825 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 732 | MKKINEGLLDSKILSAFNTVIALLGSIVIIVMNIMIIQQY | NivG.826 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 733 | MKKINEGLLDSKILSAFNTVIALLGSIVIIVMNIMIIQQY | NivG.827 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 734 | MKKINEGLLDSKILSAFNTVIALLGSIVIIVMNIMIIQQY | NivG.828 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 735 | MKKINEGLLDSKILSAFNTVIALLGSIVIIVMNIMIIQQY | NivG.829 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECNISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 736 | MKKINEGLLDSKILSAFNTVIALLGSIVIIVMNIMIIQQY | NivG.830 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 737 | MKKINEGLLDSKILSAFNTVIALLGSIVIIVMNIMIIQQY | NivG.831 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 738 | MKKINEGLLDSKILSAFNTVIALLGSIVIIVMNIMIIQQY | NivG.832 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 739 | MKKINEGLLDSKILSAFNTVIALLGSIVIIVMNIMIIQQY | NivG.833 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 740 | MKKINEGLLDSKILSAFNTVIALLGSIVIIVMNIMIIQQY | NivG.834 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 741 | MKKINEGLLDSKILSAFNTVIALLGSIVIIVMNIMIIQQY | NivG.835 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 742 | MKKINEGLLDSKILSAFNTVIALLGSIVIIVMNIMIIQQY | NivG.836 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 743 | MKKINEGLLDSKILSAFNTVIALLGSIVIIVMNIMIIQQY | NivG.837 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 744 | MKKINEGLLDSKILSAFNTVIALLGSIVIIVMNIMIIQQY | NivG.838 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 745 | MKKINEGLLDSKILSAFNTVIALLGSIVIIVMNIMIIQQY | NivG.839 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 746 | MKKINEGLLDSKILSAFNTVIALLGSIVIIVMNIMIIQQY | NivG.840 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 747 | MKKINEGLLDSKILSAFNTVIALLGSIVIIVMNIMIIQQY | NivG.841 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 748 | MKKINEGLLDSKILSAFNTVIALLGSIVIIVMNIMIIQQY | NivG.842 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 749 | MKKINEGLLDSKILSAFNTVIALLGSIVIIVMNIMIIQQY | NivG.843 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 750 | MKKINEGLLDSKILSAFNTVIALLGSIVIIVMNIMIIQQY | NivG.844 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 751 | MKKINEGLLDSKILSAFNTVIALLGSIVIIVMNIMIIQQY | NivG.845 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LNSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 752 | MKKINEGLLDSKILSAFNTVIALLGSIVIIVMNIMIIQQY | NivG.846 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 753 | MKKINEGLLDSKILSAFNTVIALLGSIVIIVMNIMIIQQY | NivG.847 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 754 | MKKINEGLLDSKILSAFNTVIALLGSIVIIVMNIMIIQQY | NivG.848 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 755 | MKKINEGLLDSKILSAFNTVIALLGSIVIIVMNIMIIQQY | NivG.849 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 756 | MKKINEGLLDSKILSAFNTVIALLGSIVIIVMNIMIIQQY | NivG.850 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 757 | MKKINEGLLDSKILSAFNTVIALLGSIVIIVMNIMIIQQY | NivG.851 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 758 | MKKINEGLLDSKILSAFNTVIALLGSIVIIVMNIMIIQQY | NivG.852 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 759 | MKKINEGLLDSKILSAFNTVIALLGSIVIIVMNIMIIQQY | NivG.853 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | NDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 760 | MKKINEGLLDSKILSAFNTVIALLGSIVIIVMNIMIIQQY | NivG.854 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 761 | MKKINEGLLDSKILSAFNTVIALLGSIVIIVMNIMIIQQY | NivG.855 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 762 | MKKINEGLLDSKILSAFNTVIALLGSIVIIVMNIMIIQQY | NivG.856 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 763 | MKKINEGLLDSKILSAFNTVIALLGSIVIIVMNIMIIQQY | NivG.857 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YNLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 764 | MKKINEGLLDSKILSAFNTVIALLGSIVIIVMNIMIIQQY | NivG.858 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 765 | MKKINEGLLDSKILSAFNTVIALLGSIVIIVMNIMIIQQY | NivG.859 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRNNTVISRPGQS | |
| | QCPRFNTCPAICAEGVYNDAFLIDRINWISAGVFLDSQ | |
| | ATAANPVFTVFKDNEILYRAQLASEDTNAQKTITNCFL | |
| | LKNKIWCISLVEIYDTGDNVIRPKLFAVKIPEQCT | |
| 766 | MKKINEGLLDSKILSAFNTVIALLGSIVIIVMNIMIIQQY | NivG.860 |
| | TRSTDNQAVIKDALQGIQQQIKGLADKIGTEIGPKVSLI | |
| | DTSSTITIPANIGLLGSKISQSTASINENVNEKCKFTLPP | |
| | LKIHECQISCPNPLPFREYRPQTEGVSNLVGLPNNICLQ | |
| | KTSNQILKPKLISYTLPVVGQSGTCITDPLLAMDEGYF | |
| | AYSHLERIGSCSRGVSKQRIIGVGEVLDRGDEVPSLFM | |
| | TNVWTPPNPNTVYHCSAVYNNEFYYVLCAVSTVGDPI | |
| | LQSTYWSGSLMMTRLAVKPKSNGGGYNQHQLALRSI | |
| | EKGRYDKVMPYGPSGIKQGDTLYFPAVGFLVRTEFKY | |
| | QDSNCPITKCQYSKPENCRLSMGIRPNSHYILRSGLLK | |
| | YQLSDGENPKVVFIEISDQRLSIGSPSKIYDSLGQPVFY | |
| | QASFSWDTMIKFGDVLTVNPLVVNWRQNTVISRPGQS | |
| | QCPRFNTCPAICAEGVYNDAFLIDRINWISAGVFLDSN | |
| | ATAANPVFTVFKDNEILYRAQLASEDTNAQKTITNCFL | |
| | LKNKIWCISLVEIYDTGDNVIRPKLFAVKIPEQCT | |
| 767 | MGFTTKIIFLYNLVLVYAGFDDPRKAIELVQKRYGRPC | BaEVTR |
| | DCSGGQVSEPPSDRVSQVTCSGKTAYLMPDQRWKCK | |
| | SIPKDTSPSGPLQECPCNSYQSSVHSSCYTSYQQCRSG | |
| | NKTYYTATLLKTQTGGTSDVQVLGSTNKLIQSPCNGI | |
| | KGQSICWSTTAPIHVSDGGGPLDTTRIKSVQRKLEEIH | |
| | KALYPELQYHPLAIPKVRDNLMVDAQTLNILNATYNL | |
| | LLMSNTSLVDDCWLCLKLGPPTPLAIPNFLLSYVTRSS | |
| | DNISCLIIPPLLVQPMQFSNSSCLFSPSYNSTEEIDLGHV | |
| | AFSNCTSITNVTGPICAVNGSVFLCGNNMAYTYLPTN | |
| | WTGLCVLATLLPDIDIIPGDEPVPIPAIDHFIYRPKRAIQ | |
| | FIPLLAGLGITAAFTTGATGLGVSVTQYTKLSNQLISDV | |
| | QILSSTIQDLQDQVDSLAEVVLQNRRGLDLLTAEQGGI | |
| | CLALQEKCCFYVNKSGIVRDKIKTLQEELERRRKDLAS | |
| | NPLWTGLQGLLPYLLPFLGPLLTLLLLLTIGPCIFNRLV | |
| | QFVKDRISVVQALVLTQQYHQLKPLEYEP | |
| 768 | MGQTVTTPLSLTLGHWKDVERIAHNQSVDVKKRRWV | MLV-gag |
| | TFCSAEWPTFNVGWPRDGTFNRDLITQVKIKVFSPGPH | |
| | GHPDQVPYIVTWEALAFDPPPWVKPFVHPKPPPPLPPS | |
| | APSLPLEPPRSTPPRSSLYPALTPSLGAKPKPQVLSDSG | |
| | GPLIDLLTEDPPPYRDPRPPPSDRDGNGGEATPAGEAP | |
| | DPSPMASRLRGRREPPVADSTTSQAFPLRAGGNGQLQ | |
| | YWPFSSSDLYNWKNNNPSFSEDPGKLTALIESVLITHQ | |
| | PTWDDCQQLLGTLLTGEEKQRVLLEARKAVRGDDGR | |
| | PTQLPNEVDAAFPLERPDWDYTTQAGRNHLVHYRQL | |
| | LLAGLQNAGRSPTNLAKVKGITQGPNESPSAFLERLKE | |
| | AYRRYTPYDPEDPGQETNVSMSFIWQSAPDIGRKLER | |
| | LEDLKNKTLGDLVREAEKIFNKRETPEEREERIRRETE | |
| | EKEERRRTEDEQKEKERDRRRHREMSKLLATVVSGQ | |
| | KQDRQGGERRRSQLDRDQCAYCKEKGHWAKDCPKK | |
| | PRGPRGPRPQTSLLTLDDSGGSLQLPPLERLTLGSLQLP | |
| | PLERLTLGSLQLPPLERLTLTSTLLMENSSGDYKDDDD | |
| | KKRTADGSEFESPKKKRKVSGGSDKKYSIGLDIGTNSV | |
| | GWAVITDEYKVPSKKFKVLGNTDRHSIKKNLIGALLF | |
| | DSGETAEATRLKRTARRRYTRRKNRICYLQEIFSNEM | |
| | AKVDDSFFHRLEESFLVEEDKKHERHPIFGNIVDEVAY | |
| | HEKYPTIYHLRKKLVDSTDKADLRLIYLALAHMIKFR | |
| | GHFLIEGDLNPDNSDVDKLFIQLVQTYNQLFEENPINA | |
| | SGVDAKAILSARLSKSRRLENLIAQLPGEKKNGLFGNL | |
| | IALSLGLTPNFKSNFDLAEDAKLQLSKDTYDDDLDNL | |
| | LAQIGDQYADLFLAAKNLSDAILLSDILRVNTEITKAPL | |
| | SASMIKRYDEHHQDLTLLKALVRQQLPEKYKEIFFDQ | |
| | SKNGYAGYIDGGASQEEFYKFIKPILEKMDGTEELLVK | |
| | LNREDLLRKQRTFDNGSIPHQIHLGELHAILRRQEDFY | |
| | PFLKDNREKIEKILTFRIPYYVGPLARGNSRFAWMTRK | |
| | SEETITPWNFEEVVDKGASAQSFIERMTNFDKNLPNEK | |
| | VLPKHSLLYEYFTVYNELTKVKYVTEGMRKPAFLSGE | |
| | QKKAIVDLLFKTNRKVTVKQLKEDYFKKIECFDSVEIS | |
| | GVEDRFNASLGTYHDLLKIIKDKDFLDNEENEDILEDI | |
| | VLTLTLFEDREMIEERLKTYAHLFDDKVMKQLKRRRY | |
| | TGWGRLSRKLINGIRDKQSGKTILDFLKSDGFANRNF | |
| | MQLIHDDSLTFKEDIQKAQVSGQGDSLHEHIANLAGSP | |
| | AIKKGILQTVKVVDELVKVMGRHKPENIVIEMARENQ | |
| | TTQKGQKNSRERMKRIEEGIKELGSQILKEHPVENTQL | |
| | QNEKLYLYYLQNGRDMYVDQELDINRLSDYDVDHIV | |
| | PQSFLKDDSIDNKVLTRSDKNRGKSDNVPSEEVVKKM | |
| | KNYWRQLLNAKLITQRKFDNLTKAERGGLSELDKAG | |
| | FIKRQLVETRQITKHVAQILDSRMNTKYDENDKLIREV | |
| | KVITLKSKLVSDFRKDFQFYKVREINNYHHAHDAYLN | |
| | AVVGTALIKKYPKLESEFVYGDYKVYDVRKMIAKSE | |
| | QEIGKATAKYFFYSNIMNFFKTEITLANGEIRKRPLIET | |
| | NGETGEIVWDKGRDFATVRKVLSMPQVNIVKKTEVQ | |
| | TGGFSKESILPKRNSDKLIARKKDWDPKKYGGFDSPT | |
| | VAYSVLVVAKVEKGKSKKLKSVKELLGITIMERSSFE | |
| | KNPIDFLEAKGYKEVKKDLIIKLPKYSLFELENGRKRM | |
| | LASAGELQKGNELALPSKYVNFLYLASHYEKLKGSPE | |
| | DNEQKQLFVEQHKHYLDEIIEQISEFSKRVILADANLD | |
| | KVLSAYNKHRDKPIREQAENIIHLFTLTNLGAPAAFKY | |
| | FDTTIDRKRYTSTKEVLDATLIHQSITGLYETRIDLSQL | |
| | GGDSGGSKRTADGSEFEPKKKRKV | |
| 769 | MSPQRDRINAFYKDNPHPKGSRIVINREHLMIDRPYVL | Measles H protein |
| | LAVLFVMFLSLIGLLAIAGIRLHRAAIYTAEIHKSLSTN | |
| | LDVTNSIEHQVKDVLTPLFKIIGDEVGLRTPQRFTDLV | |
| | KFISDKIKFLNPDREYDFRDLTWCINPPERIKLDYDQY | |
| | CADVAAEELMNALVNSTLLETRTTNQFLAVSKGNCS | |
| | GPTTIRGQFSNMSLSLLDLYLGRGYNVSSIVTMTSQG | |
| | MYGGTYLVEKPNLSSKRSELSQLSMYRVFEVGVIRNP | |
| | GLGAPVFHMTNYLEQPVSNDLSNCMVALGELKLAAL | |
| | CHGEDSITIPYQGSGKGVSFQLVKLGVWKSPTDMQSW | |
| | VPLSTDD- | |
| | PVIDRLYLSSHRGVIADNQAKWAVPTTRTDDKLRMET | |
| | CFQQACKGKIQALCENPEWAPLKDNRIPSYGVLSVDL | |
| | SLTVELKIKIASGFGPLITHGSGMDLYKSNHNNVYWLT | |
| | IPPMKNLALGVINTLEWIPRFKVSPYLFNVPIKEAGEDC | |
| | HAPTYLPAEVDGDVKLSSNLVILPGQDLQYVLATYDT | |
| | SRVEHAVVYYVYSPSRSFSYFYPFRLPIKGVPIELQVEC | |
| | FTWDQKLWCRHFCVLADSESGGHITHSGMEGMGVSC | |
| | TVTREDGTNRR | |
| 770 | MLPYQDKVGAFYKDNARANSTKLSLVTEGHGGRRPP | Canine distemper virus H |
| | YLLFVLLILLVGILALLAITGVRFHQVSTSNMEFSRLLK | protein |
| | EDMEKSEAVHHQVIDVLTPLFKIIGDEIGLRLPQKLNEI | |
| | KQFILQKTNFFNPNREFDFRDLHWCINPPSTVKVNFTN | |
| | YCESIGIRKAIASAANPILLSALSGGRGDIFPPHRCSGAT | |
| | TSVGKVFPLSVSLSMSLISRTSEVINMLTAISDGVYGKT | |
| | YLLVPDDIEREFDTREIRVFEIGFIKRWLNDMPLLQTTN | |
| | YMVLPKNSKAKVCTIAVGELTLASLCVEESTVLLYHD | |
| | SSGSQDGILVVTLGIFWATPMDHIEEVIPVAHPSMKKI | |
| | HITNHRGFIKDSIATWMVPALASEKQEEQKGCLESAC | |
| | QRKTYPMCNQASWEPFGGRQLPSYGRLTLPLDASVDL | |
| | QLNISFTYGPVILNGDGMDYYESPLLNSGWLTIPPKDG | |
| | TISGLINKAGRGDQFTVLPHVLTFAPRESSGNCYLPIQT | |
| | SQIRDRDVLIESNIVVLPTQSIRYVIATYDISRSDHAIVY | |
| | YVYDPIRTISYTHPFRLTTKGRPDFLRIECFVWDDNLW | |
| | CHQF | |

What is claimed:

1. A lipid particle, comprising:
(a) a retargeted attachment protein comprising a paramyxovirus envelope attachment protein linked to (i) a first targeting moiety directed to a first target molecule expressed on the surface of a target cell, and (ii) a second targeting moiety directed to a second target molecule expressed on the surface of a target cell; and
(b) at least one paramyxovirus fusion (F) protein; and
wherein the protein in (a), and (b) are exposed on the outside of the lipid bilayer.

2. The lipid particle of claim 1, wherein the paramyxovirus attachment protein is a variant paramyxovirus envelope attachment protein comprising one or more mutations to reduce native tropism relative to the wild-type paramyxovirus envelope attachment protein not comprising the one or more mutations.

3. The lipid particle of claim 1 or 2, wherein the paramyxovirus envelope attachment protein is a first paramyxovirus envelope attachment protein and the lipid particle further comprises a second paramyxovirus envelope attachment protein, wherein the second paramyxovirus envelope attachment protein is a variant paramyxovirus envelope attachment protein comprising one or more mutations to reduce native tropism relative to the wild-type paramyxovirus envelope attachment protein not comprising the one or more mutations, wherein the second paramyxovirus envelope attachment protein is exposed on the outside of the lipid bilayer.

4. The lipid particle of any one of claims 1-3, wherein the targeting one or both of the first target molecule and the second target molecule does not activate or inhibit, induce a phenotype change (for example maturation and/or differentiation), induce proliferation, and/or induce apoptosis of said target cell.

5. A lipid particle, comprising:
(a) a retargeted attachment protein comprising a first paramyxovirus envelope attachment protein operably linked to (i) a first targeting moiety directed to a first target molecule expressed on the surface of a target cell, and (ii) a second targeting moiety directed to a second target molecule expressed on the surface of a target cell,
(b) a second paramyxovirus envelope attachment protein that is a variant paramyxovirus envelope attachment protein comprising one or more amino acid substitutions to reduce the native tropism relative to the wild-type paramyxovirus envelope attachment protein not comprising the one or more amino acid substitutions; and
(c) at least one paramyxovirus fusion (F) protein;
wherein the protein in (a), (b) and (c) are exposed on the outside of the lipid bilayer.

6. The lipid particle of any of claims 1-5, wherein the first paramyxovirus envelope attachment protein is a variant paramyxovirus envelope attachment protein.

7. The lipid particle of claim 6, wherein the variant paramyxovirus envelope attachment protein comprises one or more amino acid substitutions that reduces native tropism relative to the wild-type paramyxovirus envelope attachment protein not comprising the one or more amino acid substitutions.

8. The lipid particle of any of claims 1-7, wherein the first and second paramyxovirus envelope attachment protein are the same.

9. The lipid particle of any of claims 1-8, wherein the second paramyxovirus envelope attachment protein is not linked or fused to a non-viral heterologous moiety that is a cell-specific targeting domain or functional domain, optionally wherein the second paramyxovirus envelope attachment protein is not linked or fused to a cell-specific targeting domain that is a targeting moiety directed to a target molecule expressed on the surface of a target cell.

10. The lipid particle of claims 1-9, wherein the first and second targeting moiety are each not selected from the group consisting of a cytokine, growth factor, hormone, neurotransmitter, apoptosis ligand and their combinations.

11. The lipid particle of any of claims 1-10, wherein targeting one or both of the first target molecule and the second target molecule does not modulate or induce a signal in the target cell.

12. The lipid particle of any of claims 1-11, wherein the first and second targeting moieties each bind to a cell surface molecule present on a target cell.

13. The lipid particle of any of claims 1-12, wherein the first targeting moiety binds to a cell surface molecule present on a first target cell, and the second targeting moiety binds a surface molecule present on a second target cell.

14. The lipid particle of any of claims 1-13, wherein the first and second target molecule are different target molecules.

15. The lipid particle of any of claims 1-13, wherein the first and second target molecule are the same target molecule.

16. The lipid particle of claim 15, wherein the first and second targeting moiety bind distinct epitopes of the same target molecule.

17. The lipid particle of any of claims 1-16, wherein the cell surface molecule is a protein, glycan, or lipid.

18. The lipid particle of any of claims 1-17, wherein the target cell is selected from the group consisting of tumor-infiltrating lymphocytes, T cells, neoplastic or tumor cells, virus-infected cells, stem cells, central nervous system (CNS) cells, hematopoietic stem cells (HSCs), and liver cells.

19. The lipid particle of any of claims 1-18, wherein the target cell is selected from the group consisting of a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+B cell, a CD19+B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

20. The lipid particle of any of claims 1-19, wherein the target cell is a hepatocyte.

21. The lipid particle of claim 20, wherein the cell surface molecule is selected from the group consisting of CD34, CD117, and CD133.

22. The lipid particle of any of claims 1-19, wherein the target cell is a T cell.

23. The lipid particle of claim 22, wherein the cell surface molecule is selected from the group consisting of CD3, CD4, CD7 CD8, ASCT2, CD105, CD110, CD117, CD133, CD146, CD164, CD34, CD46, CD49f, CD90, EPCR, and ITGA3.

24. The lipid particle of any of claims 5-23, wherein the second paramyxovirus envelope attachment protein that is a variant paramyxovirus envelope attachment protein comprises one or more amino acid substitutions corresponding to amino acid substitutions selected from the group consisting of E501A, W504A, Q530A and E533A with reference to numbering set forth in SEQ ID NO:1.

25. A lipid particle, comprising:
(a) a first retargeted attachment protein comprising (i) a first paramyxovirus envelope attachment protein; and (ii) a first targeting moiety directed to CD133; and a second targeting moiety directed to CD133; and
(b) at least one paramyxovirus fusion (F) protein; and
the protein in (a), and (b) are exposed on the outside of the lipid bilayer.

26. The lipid particle of any of claims 1-25, wherein:
(i) the first targeting moiety is an scFv and comprises an amino acid sequence selected from the group consisting of SEQ ID NOs: 516, 525, 534, 543, and 552, or an amino acid sequence having at least 90%, 91%, 92%, 93%, 94%, 95%, 96%, 97%, 98%, or 99% sequence identity thereto; and/or
(ii) the second targeting moiety is an scFv and comprises an amino acid sequence selected from the group consisting of SEQ ID NOs: 516, 525, 534, 543, and 552, or an amino acid sequence having at least 90%, 91%, 92%, 93%, 94%, 95%, 96%, 97%, 98%, or 99% sequence identity thereto.

27. The lipid particle of any of claims 1-25, wherein:
(i) the first targeting moiety comprises: (a) a CD133 binding agent comprising a CDR-H1, a CDR-H2, and a CDR-H3 comprising the amino acid sequences of SEQ ID NOs: 536, 537, and 538, respectively, and a CDR-L1, a CDR-L2, and a CDR-L3 comprising the amino acid sequences of SEQ ID NOs: 540, 541, and 542, respectively; (b) a CD133 binding agent comprising a CDR-H1, a CDR-H2, and a CDR-H3 comprising the amino acid sequences of SEQ ID NOs: 545, 546, and 547, respectively, and a CDR-L1, a CDR-L2, and a CDR-L3 comprising the amino acid sequences of SEQ ID NOs: 549, 550, and 551, respectively; (c) a CD133 binding agent comprising a CDR-H1, a CDR-H2, and a CDR-H3 comprising the amino acid sequences of SEQ ID NOs: 518, 519, and 520, respectively, and a CDR-L1, a CDR-L2, and a CDR-L3 comprising the amino acid sequences of SEQ ID NOs: 522, 523, and 524, respectively; (d) a CD133 binding agent comprising a CDR-H1, a CDR-H2, and a CDR-H3 comprising the amino acid sequences of SEQ ID NOs: 527, 528, and 529, respectively, and a CDR-L1, a CDR-L2, and a CDR-L3 comprising the amino acid sequences of SEQ ID NOs: 531, 532, and 533, respectively; or (e) a CD133 binding agent comprising a CDR-H1, a CDR-H2, and a CDR-H3 comprising the amino acid sequences of SEQ ID NOs: 554, 555, and 556, respectively, and a CDR-L1, a CDR-L2, and a CDR-L3 comprising the amino acid sequences of SEQ ID NOs: 558, 559, and 560, respectively; and/or
(ii) the second targeting moiety comprises: (a) a CD133 binding agent comprising a CDR-H1, a CDR-H2, and a CDR-H3 comprising the amino acid sequences of SEQ ID NOs: 536, 537, and 538, respectively, and a CDR-L1, a CDR-L2, and a CDR-L3 comprising the amino acid sequences of SEQ ID NOs: 540, 541, and 542, respectively; (b) a CD133 binding agent comprising a CDR-H1, a CDR-H2, and a CDR-H3 comprising the amino acid sequences of SEQ ID NOs: 545, 546, and 547, respectively, and a CDR-L1, a CDR-L2, and a CDR-L3 comprising the amino acid sequences of SEQ ID NOs: 549, 550, and 551, respectively; (c) a CD133 binding agent comprising a CDR-H1, a CDR-H2, and a CDR-H3 comprising the amino acid sequences of SEQ ID NOs: 518, 519, and 520, respectively, and a CDR-L1, a CDR-L2, and a CDR-L3 comprising the amino acid sequences of SEQ ID NOs: 522, 523, and 524, respectively; (d) a CD133 binding agent comprising a CDR-H1, a CDR-H2, and a CDR-H3 comprising the amino acid sequences of SEQ ID NOs: 527, 528, and 529, respectively, and a CDR-L1, a CDR-L2, and a CDR-L3 comprising the amino acid sequences of SEQ ID NOs: 531, 532, and 533, respectively; or (e) a CD133 binding agent comprising a CDR-H1, a CDR-H2, and a CDR-H3 comprising the amino acid sequences of SEQ ID NOs: 554, 555, and 556, respectively, and a CDR-L1, a CDR-L2, and a CDR-L3 comprising the amino acid sequences of SEQ ID NOs: 558, 559, and 560, respectively.

28. The lipid particle of any of claims 1-25, wherein:
(i) the first targeting moiety comprises: (a) a CD133 binding agent comprising a CDR-H1, a CDR-H2, and a CDR-H3 comprising the amino acid sequences of SEQ ID NOs: 289, 565, and 538, respectively, and a CDR-L1, a CDR-L2, and a CDR-L3 comprising the amino acid sequences of SEQ ID NOs: 540, 541, and 542, respectively; (b) a CD133 binding agent comprising a CDR-H1, a CDR-H2, and a CDR-H3 comprising the amino acid sequences of SEQ ID NOs: 566, 567, and 547, respectively, and a CDR-L1, a CDR-L2, and a CDR-L3 comprising the amino acid sequences of SEQ ID NOs: 549, 550, and 551, respectively; (c) a CD133 binding agent comprising a CDR-H1, a CDR-H2, and a CDR-H3 comprising the amino acid sequences of SEQ ID NOs: 568, 569, and 520, respectively, and a CDR-L1, a CDR-L2, and a CDR-L3 comprising the amino acid sequences of SEQ ID NOs: 522, 523, and 524, respectively; (d) a CD133 binding agent comprising a CDR-H1, a CDR-H2, and a CDR-H3 comprising the amino acid sequences of SEQ ID NOs: 570, 571, and 529, respectively, and a CDR-L1, a CDR-L2, and a CDR-L3 comprising the amino acid sequences of SEQ ID NOs: 531, 532, and 533, respectively; or (e) a CD133 binding agent comprising a CDR-H1, a CDR-H2, and a CDR-H3 comprising the amino acid sequences of SEQ ID NOs: 572, 573, and 556, respectively, and a CDR-L1, a CDR-L2, and a CDR-L3 comprising the amino acid sequences of SEQ ID NOs: 558, 559, and 560, respectively; and/or
(ii) the second targeting moiety comprises: (a) a CD133 binding agent comprising a CDR-H1, a CDR-H2, and a CDR-H3 comprising the amino acid sequences of SEQ ID NOs: 289, 565, and 538, respectively, and a CDR-L1, a CDR-L2, and a CDR-L3 comprising the amino acid sequences of SEQ ID NOs: 540, 541, and 542, respectively; (b) a CD133 binding agent comprising a CDR-H1, a CDR-H2, and a CDR-H3 comprising the amino acid sequences of SEQ ID NOs: 566, 567, and 547, respectively, and a CDR-L1, a CDR-L2, and a CDR-L3 comprising the amino acid sequences of SEQ ID NOs: 549, 550, and 551, respectively; (c) a CD133 binding agent comprising a CDR-H1, a CDR-H2, and a CDR-H3 comprising the amino acid sequences of SEQ ID NOs: 568, 569, and 520, respectively, and a CDR-L1, a CDR-L2, and a CDR-L3 comprising the amino acid sequences of SEQ ID NOs: 522, 523, and 524, respectively; (d) a CD133 binding agent comprising a CDR-H1, a CDR-H2, and a CDR-H3 comprising the amino acid sequences of SEQ ID NOs: 570, 571, and 529, respectively, and a CDR-L1, a CDR-L2, and a CDR-L3 comprising the amino acid sequences of SEQ ID NOs: 531, 532, and 533, respectively; or (e) a CD133 binding agent comprising a CDR-H1, a CDR-H2, and a CDR-H3 comprising the amino acid sequences of SEQ ID NOs: 572, 573, and 556, respectively, and a CDR-L1, a CDR-L2, and a CDR-L3 comprising the amino acid sequences of SEQ ID NOs: 558, 559, and 560, respectively.

29. The lipid particle of any of claims 1-25, wherein:
(i) the first targeting moiety comprises: (a) a CD133 binding agent comprising a heavy chain variable (VH) region comprising the amino acid sequence of SEQ ID NO: 535, or an amino acid sequence having at least 90%, 91%, 92%, 93%, 94%, 95%, 96%, 97%, 98%, or 99% sequence identity thereto; and a light chain variable (VL) region comprising the amino acid sequence of SEQ ID NO: 539, or an amino acid sequence having at least 90%, 91%, 92%, 93%, 94%, 95%, 96%, 97%, 98%, or 99% sequence identity thereto; (b) a CD133 binding agent comprising a VH region comprising the amino acid sequence of SEQ ID NO: 544, or an amino acid sequence having at least 90%, 91%, 92%, 93%, 94%, 95%, 96%, 97%, 98%, or 99% sequence identity thereto; and a VL region comprising the amino acid sequence of SEQ ID NO: 548, or an amino acid sequence having at least 90%, 91%, 92%, 93%, 94%, 95%, 96%, 97%, 98%, or 99% sequence identity thereto; (c) a CD133 binding agent comprising a VH region comprising the amino acid sequence of SEQ ID NO: 517, or an amino acid sequence having at least 90%, 91%, 92%, 93%, 94%, 95%, 96%, 97%, 98%, or 99% sequence identity thereto; and a VL region comprising the amino acid sequence of SEQ ID NO: 521, or an amino acid sequence having at least 90%, 91%, 92%, 93%, 94%, 95%, 96%, 97%, 98%, or 99% sequence identity thereto; (d) a CD133 binding agent comprising a VH region comprising the amino acid sequence of SEQ ID NO: 526, or an amino acid sequence having at least 90%, 91%, 92%, 93%, 94%, 95%, 96%, 97%, 98%, or 99% sequence identity thereto; and a VL region comprising the amino acid sequence of SEQ ID NO: 530, or an amino acid sequence having at least 90%, 91%, 92%, 93%, 94%, 95%, 96%, 97%, 98%, or 99% sequence identity thereto; or (e) a CD133 binding agent comprising a VH region comprising the amino acid sequence of SEQ ID NO: 553, or an amino acid sequence having at least 90%, 91%, 92%, 93%, 94%, 95%, 96%, 97%, 98%, or 99% sequence identity thereto; and a VL region comprising the amino acid sequence of SEQ ID NO: 557, or an amino acid sequence having at least 90%, 91%, 92%, 93%, 94%, 95%, 96%, 97%, 98%, or 99% sequence identity thereto; and/or
(ii) the second targeting moiety comprises: (a) a CD133 binding agent comprising a heavy chain variable (VH) region comprising the amino acid sequence of SEQ ID NO: 535, or an amino acid sequence having at least 90%, 91%, 92%, 93%, 94%, 95%, 96%, 97%, 98%, or 99% sequence identity thereto; and a light chain variable (VL) region comprising the amino acid sequence of SEQ ID NO: 539, or an amino acid sequence having at least 90%, 91%, 92%, 93%, 94%, 95%, 96%, 97%, 98%, or 99% sequence identity thereto; (b) a CD133 binding agent comprising a VH region comprising the amino acid sequence of SEQ ID NO: 544, or an amino acid sequence having at least 90%, 91%, 92%, 93%, 94%, 95%, 96%, 97%, 98%, or 99% sequence identity thereto; and a VL region comprising the amino acid sequence of SEQ ID NO: 548, or an amino acid sequence having at least 90%, 91%, 92%, 93%, 94%, 95%, 96%, 97%, 98%, or 99% sequence identity thereto; (c) a CD133 binding agent comprising a VH region comprising the amino acid sequence of SEQ ID NO: 517, or an amino acid sequence having at least 90%, 91%, 92%, 93%, 94%, 95%, 96%, 97%, 98%, or 99% sequence identity thereto; and a VL region comprising the amino acid sequence of SEQ ID NO: 521, or an amino acid sequence having at least 90%, 91%, 92%, 93%, 94%, 95%, 96%, 97%, 98%, or 99% sequence identity thereto; (d) a CD133 binding agent comprising a VH region comprising the amino acid sequence of SEQ ID NO: 526, or an amino acid sequence having at least 90%, 91%, 92%, 93%, 94%, 95%, 96%, 97%, 98%, or 99% sequence identity thereto; and a VL region comprising the amino acid sequence of SEQ ID NO: 530, or an amino acid sequence having at least 90%, 91%, 92%, 93%, 94%, 95%, 96%, 97%, 98%, or 99% sequence identity thereto; or (e) a CD133 binding agent comprising a VH region comprising the amino acid sequence of SEQ ID NO: 553, or an amino acid sequence having at least 90%, 91%, 92%, 93%, 94%, 95%, 96%, 97%, 98%, or 99% sequence identity thereto; and a VL region comprising the amino acid sequence of SEQ ID NO: 557, or an amino acid sequence having at least 90%, 91%, 92%, 93%, 94%, 95%, 96%, 97%, 98%, or 99% sequence identity thereto.

30. The lipid particle of any of claims 1-29, wherein
(a) the first targeting moiety comprises a CD133 binding agent comprising a VH region comprising the amino acid sequence of SEQ ID NO: 517, or an amino acid sequence having at least 90%, 91%, 92%, 93%, 94%, 95%, 96%, 97%, 98%, or 99% sequence identity thereto; and
(b) the second targeting moiety comprises a CD133 binding agent comprising a VH region comprising the amino acid sequence of SEQ ID NO: 526, or an amino acid sequence having at least 90%, 91%, 92%, 93%, 94%, 95%, 96%, 97%, 98%, or 99% sequence identity thereto.

31. The lipid particle of any of claims 1-29, wherein:
(a) the first targeting moiety comprises a CD133 binding agent comprising a VH region comprising the amino acid sequence of SEQ ID NO: 526, or an amino acid sequence having at least 90%, 91%, 92%, 93%, 94%, 95%, 96%, 97%, 98%, or 99% sequence identity thereto; and
(b) the second targeting moiety comprises a CD133 binding agent comprising a VH region comprising the amino acid sequence of SEQ ID NO: 517, or an amino acid sequence having at least 90%, 91%, 92%, 93%, 94%, 95%, 96%, 97%, 98%, or 99% sequence identity thereto.

32. The lipid particle of any of claims 1-31, wherein the first and second targeting moiety bind to distinct epitopes on CD133.

33. The lipid particle of any of claims 25-32, further comprising a second paramyxovirus envelope attachment protein that is a variant paramyxovirus envelope attachment protein comprising one or more amino acid substitutions to reduce the native tropism relative to the wild-type paramyxovirus envelope attachment protein not comprising the one or more amino acid substitutions.

34. The lipid particle of claim 33, wherein the second paramyxovirus envelope attachment protein that is a variant paramyxovirus envelope attachment protein comprises one or more amino acid substitutions corresponding to amino acid substitutions selected from the group consisting of E501A, W504A, Q530A and E533A with reference to numbering set forth in SEQ ID NO:1.

35. A lipid particle, comprising:
(a) a first retargeted attachment protein comprising (i) a first paramyxovirus envelope attachment protein; and (ii) a first targeting moiety directed to CD117; and a second targeting moiety directed to CD117; and
(b) at least one paramyxovirus fusion (F) protein; and
the protein in (a), (b) are exposed on the outside of the lipid bilayer.

36. The lipid particle of claims 1-25 and 35, wherein
(a) the first targeting moiety comprises a CD117 binding agent comprising a VHH single domain antibody comprising a CDR-H1, a CDR-H2, and a CDR-H3 contained within an amino acid sequence selected from the group consisting of SEQ ID NOs: 512-515; and/or wherein
(b) the second targeting moiety comprises a CD117 binding agent comprising a VHH single domain antibody comprising a CDR-H1, a CDR-H2, and a CDR-H3 contained within an amino acid sequence selected from the group consisting of SEQ ID NOs: 512-515.

37. The lipid particle of any of claims 1-25, and 35-36, wherein
(a) the first targeting moiety comprising a VHH single domain antibody comprising an amino acid sequence selected from the group consisting of SEQ ID NOs: 512-515, or an amino acid sequence having at least 90%, 91%, 92%, 93%, 94%, 95%, 96%, 97%, 98%, or 99% sequence identity thereto; and/or
(b)) the second targeting moiety comprising a VHH single domain antibody comprising an amino acid sequence selected from the group consisting of SEQ ID NOs: 512-515, or an amino acid sequence having at least 90%, 91%, 92%, 93%, 94%, 95%, 96%, 97%, 98%, or 99% sequence identity thereto.

38. The lipid particle of claim 36 or claim 37, wherein
(a) the first targeting moiety comprises a VHH comprising the amino acid sequence set forth in any one of SEQ ID NOS: 512-515; and/or
(b) the second targeting moiety comprises a VHH comprising the amino acid sequence set forth in any one of SEQ ID NOS: 512-515.

39. The lipid particle of any of claims 1-25, and 35-38, wherein the first and second targeting moiety bind to distinct epitopes on CD117.

40. The lipid particle of any of claims 35-39, further comprising a second paramyxovirus envelope attachment protein that is a variant paramyxovirus envelope attachment protein comprising one or more amino acid substitutions to reduce the native tropism relative to the wild-type paramyxovirus envelope attachment protein not comprising the one or more amino acid substitutions.

41. The lipid particle of claim 40, wherein the second paramyxovirus envelope attachment protein that is a variant paramyxovirus envelope attachment protein comprises one or more amino acid substitutions corresponding to amino acid substitutions selected from the group consisting of E501A, W504A, Q530A and E533A with reference to numbering set forth in SEQ ID NO:1.

42. A lipid particle, comprising:
(a) a first retargeted attachment protein comprising (i) a first paramyxovirus envelope attachment protein; and (ii) a first targeting moiety directed to CD8; and a second targeting moiety directed to CD8; and
(b) at least one paramyxovirus fusion (F) protein; and
the protein in (a), and (b) are exposed on the outside of the lipid bilayer.

43. The lipid particle of claim 42, wherein
(i) the first targeting moiety is an scFv and comprises an amino acid sequence selected from the group consisting of SEQ ID NOs: 489, 496, 501, or 508 or an amino acid sequence having at least 90%, 91%, 92%, 93%, 94%, 95%, 96%, 97%, 98%, or 99% sequence identity thereto; and/or
(ii) the second targeting moiety is an scFv and comprises an amino acid sequence selected from the group consisting of SEQ ID NOs: 489, 496, 501, or 508, or an amino acid sequence having at least 90%, 91%, 92%, 93%, 94%, 95%, 96%, 97%, 98%, or 99% sequence identity thereto.

44. The lipid particle of claims 1-25 and 42, wherein
(a) the first targeting moiety comprises a CD8 binding agent comprising a VHH single domain antibody comprising a CDR-H1, a CDR-H2, and a CDR-H3 contained within an amino acid sequence of SEQ ID NO: 377; and/or wherein
(b) the second targeting moiety comprises a CD8 binding agent comprising a VHH single domain antibody comprising a CDR-H1, a CDR-H2, and a CDR-H3 contained within an amino acid sequence of SEQ ID NO: 377.

45. The lipid particle of claim 42 or 44, wherein
(a) the first targeting moiety comprises a VHH comprising the amino acid sequence set forth in SEQ ID NOS: 377; and/or
(b) the second targeting moiety comprises a VHH comprising the amino acid sequence set forth in SEQ ID NO: 377.

46. The lipid particle of any of claims 42-45, wherein
(a) the first targeting moiety comprises a VHH comprising the amino acid sequence set forth in SEQ ID NO: 377 or an amino acid sequence having at least 90%, 91%, 92%, 93%, 94%, 95%, 96%, 97%, 98%, or 99% sequence identity thereto; and/or
(b) the second targeting moiety is an scFv and comprises an amino acid sequence selected from the group consisting of SEQ ID NO: 501 or an amino acid sequence having at least 90%, 91%, 92%, 93%, 94%, 95%, 96%, 97%, 98%, or 99% sequence identity thereto.

47. The lipid particle of any of claims 42-45, wherein
(a) the first targeting moiety is an scFv and comprises an amino acid sequence selected from the group consisting of SEQ ID NO: 501 or an amino acid sequence having at least 90%, 91%, 92%, 93%, 94%, 95%, 96%, 97%, 98%, or 99% sequence identity thereto; and
(b) the second targeting moiety comprises a VHH comprising the amino acid sequence set forth in SEQ ID NO: 377 or an amino acid sequence having at least 90%, 91%, 92%, 93%, 94%, 95%, 96%, 97%, 98%, or 99% sequence identity thereto.

48. The lipid particle of any of claims 1-24, and 42-47, wherein the first and second targeting moiety bind to distinct epitopes on CD8.

49. The lipid particle of any of claims 16-48, wherein the distinct epitopes are non-overlapping.

50. The lipid particle of any of claims 16-49, wherein the first and second targeting moiety bind to the distinct epitopes in a non-competitive manner.

51. The lipid particle of claim 1-50, wherein each of the first targeting moiety and the second targeting moiety are independently selected from the group consisting of an antibody or antigen-binding fragment, a DARPin, an Aptamer, an Affimer, an Affibody, a Knottin, an Avimer, a Monobody, an Anticalin, a Fynomer, and a targeting peptide.

52. The lipid particle of any of claims 1-51, wherein the first targeting moiety and the second targeting moiety are independently selected from the group consisting of a single domain antibody or a single chain variable fragment (scFv).

53. The lipid particle of any of claims 42-52, further comprising a second paramyxovirus envelope attachment protein that is a variant paramyxovirus envelope attachment protein comprising one or more amino acid substitutions to reduce the native tropism relative to the wild-type paramyxovirus envelope attachment protein not comprising the one or more amino acid substitutions.

54. The lipid particle of claim 49, wherein the second paramyxovirus envelope attachment protein that is a variant paramyxovirus envelope attachment protein comprises one or more amino acid substitutions corresponding to amino acid substitutions selected from the group consisting of E501A, W504A, Q530A and E533A with reference to numbering set forth in SEQ ID NO:1.

55. A lipid particle, comprising:
(a) a retargeted attachment protein comprising (i) a first paramyxovirus envelope attachment protein; and (ii) a first and second targeting moiety directed to CD133,
(b) a second paramyxovirus envelope attachment protein that is a variant paramyxovirus envelope attachment protein comprising one or more amino acid substitutions that reduces the native tropism relative to the wild-type paramyxovirus envelope attachment protein not comprising the one or more amino acid substitutions; and
(c) at least one paramyxovirus fusion (F) protein; and
wherein the protein in (a), (b) and (c) are exposed on the outside of the lipid bilayer.

56. A lipid particle, comprising:
(a) a retargeted attachment protein comprising (i) a first paramyxovirus envelope attachment protein; and (ii) a first and second targeting moiety directed to CD117,
(b) a second paramyxovirus envelope attachment protein that is a variant paramyxovirus envelope attachment protein comprising one or more amino acid substitutions that reduces the native tropism relative to the wild-type paramyxovirus envelope attachment protein not comprising the one or more amino acid substitutions; and
(c) at least one paramyxovirus fusion (F) protein; and
wherein the protein in (a), (b) and (c) are exposed on the outside of the lipid bilayer.

57. A lipid particle, comprising:
(a) a retargeted attachment protein comprising (i) a first paramyxovirus envelope attachment protein; and (ii) a first and second targeting moiety directed to CD8,
(b) a second paramyxovirus envelope attachment protein that is a variant paramyxovirus envelope attachment protein comprising one or more amino acid substitutions that reduces the native tropism relative to the wild-type paramyxovirus envelope attachment protein not comprising the one or more amino acid substitutions; and
(c) at least one paramyxovirus fusion (F) protein; and
wherein the protein in (a), (b) and (c) are exposed on the outside of the lipid bilayer.

58. The lipid particle of claims 24-57, wherein the first paramyxovirus envelope attachment protein is a variant paramyxovirus envelope attachment protein.

59. The lipid particle of claim 58, wherein the variant paramyxovirus envelope attachment protein comprises one or more amino acid substitutions that reduces native tropism relative to the wild-type paramyxovirus envelope attachment protein not comprising the one or more amino acid substitutions.

60. The lipid particle of any of claims 25-59, wherein the second paramyxovirus envelope attachment protein is a variant paramyxovirus envelope attachment protein.

61. The lipid particle of claim 60, wherein the variant paramyxovirus envelope attachment protein comprises one or more amino acid substitutions that reduces native tropism relative to the wild-type paramyxovirus envelope attachment protein not comprising the one or more amino acid substitutions.

62. The lipid particle of any of claims 1-61, wherein the first targeting moiety is selected from the group consisting of a single domain antibody or a single chain variable fragment (scFv).

63. The lipid particle of any of claims 1-62, wherein the second targeting moiety is selected from the group consisting of a single domain antibody or a single chain variable fragment (scFv).

64. The lipid particle of claim 54 and claim 62, or 63, wherein the single domain antibody is a VHH.

65. The lipid particle of any of claims 1-64, wherein the first variant paramyxovirus envelope attachment protein and the second variant paramyxovirus envelope attachment protein are the same.

66. The lipid particle of any of claims 1-65, wherein the first paramyxovirus envelope attachment protein and the second paramyxovirus envelope attachment protein are different.

67. The lipid particle of any of claims 1-66, wherein the first paramyxovirus envelope attachment protein is an envelope attachment protein from a Nipah virus, Hendra virus, or Measles virus, or is a variant or biologically active portion thereof of any of the foregoing.

68. The lipid particle of any of claims 1-67, wherein the first paramyxovirus envelope attachment protein is a wild-type paramyxovirus G protein, H protein or HN protein or is a variant or biologically active portion of any of the foregoing.

69. The lipid particle of claim 67 or claim 68, wherein the first paramyxovirus envelope attachment protein is a wild-type Nipah virus G (NiV-G) protein or is a variant or biologically active portion of a NiV-G.

70. The lipid particle of any of claims 67-69, wherein the first paramyxovirus envelope attachment protein is a variant NiV-G that is a variant or a biologically active portion of a wild-type NiV-G.

71. The lipid particle of any of claims 1-70, wherein the second paramyxovirus envelope attachment protein is an envelope attachment protein from a Nipah virus, Hendra virus, or Measles virus, or is a variant or biologically active portion of any of the foregoing.

72. The lipid particle of any of claims 1-71, wherein the second paramyxovirus envelope attachment protein is a wild-type paramyxovirus G protein, H protein or HN protein or is a variant or biologically active portion of any of the foregoing.

73. The lipid particle of claim 71 or claim 72, wherein the second paramyxovirus envelope attachment protein is a wild-type Nipah virus G (NiV-G) protein or is a variant or a biologically active portion of a NiV-G.

74. The lipid particle of any of claims 1-73, wherein the second paramyxovirus envelope attachment protein is a variant NiV-G that is a variant or a biologically active portion of a wild-type NiV-G.

75. The lipid particle of any of claims 3-70, wherein the second paramyxovirus envelope attachment protein is a variant paramyxovirus envelope glycoprotein from a Nipah virus, Hendra virus, or Measles virus or a biologically active portion thereof.

76. The lipid particle of any of claims 3-70 and 75, wherein the second paramyxovirus envelope attachment protein is a variant of a wild-type paramyxovirus G protein, H protein or HN protein or a biologically active portion thereof.

77. The lipid particle of claim 73 or 74, wherein the variant is a variant NiV-G that is a variant of a wild-type Nipah virus G (NiV-G) protein or a biologically active portion thereof.

78. The lipid particle of claim 70, claim 74, or claim 77, wherein the variant NiV-G is truncated by up to 40 contiguous amino acids at or near the N-terminus of the wild-type NiV-G set forth in SEQ ID NO:1.

79. The lipid particle of any of claims 70, 74, 77 and 78, wherein the variant NiV-G has a truncation of amino acids 2-34 of the wild-type NiV-G set forth in SEQ ID NO:1.

80. The lipid particle of any of claims 70, 73 and 77-79, wherein the variant NiV-G exhibits reduced binding to Ephrin B2 or Ephrin B3.

81. The lipid particle of claim 80, wherein the variant NiV-G comprises: one or more amino acid substitutions corresponding to amino acid substitutions selected from the group consisting of E501A, W504A, Q530A and E533A with reference to numbering set forth in SEQ ID NO:1.

82. The lipid particle of claim 80 or claim 81, wherein the variant NiV-G comprises amino acid substitutions E501A, W504A, Q530A and E533A with reference to numbering set forth in SEQ ID NO: 1.

83. The lipid particle of any of claims 70, 74 and 77-79, wherein the variant NiV-G has an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:228.

84. The lipid particle of any of claims 70, 74 and 77-79, wherein the variant NiV-G has the amino acid sequence set forth in SEQ ID NO:228.

85. The lipid particle of any of claims 1-84, wherein the at least one paramyxovirus fusion (F) protein is an F protein from a henipavirus or is a biologically active portion thereof or variant thereof.

86. The lipid particle of claim 85, wherein the henipavirus is a Hendra virus.

87. The lipid particle of claim 85, wherein the henipavirus is a Nipah virus.

88. The lipid particle of any of claims 1-87, wherein the paramyxovirus F protein is a wild-type NiV-F protein or a variant or a biologically active portion thereof.

89. The lipid particle of any of claims 1-88, wherein the paramyxovirus F protein is a variant NiV-F that is a variant or a biologically active portion of a wild-type NiV-F protein.

90. The lipid particle of claim 89, wherein the variant NiV-F is truncated by up to 22 contiguous amino acids the at the C-terminus of the wild-type NiV-F set forth in SEQ ID NO:235, optionally not include the initial methionine.

91. The lipid particle of claim 89 or claim 90, wherein the variant NiV-F protein is a truncated NiV-F that lacks amino acids 525-546 of SEQ ID NO:235.

92. The lipid particle of any of claims 89-91, wherein the variant NiV-F has the amino acid sequence set forth in SEQ ID NO: 227 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:227.

93. The lipid particle of any of claims 89-92, wherein the variant NiV-F has the amino acid sequence set forth in SEQ ID NO:227.

94. The lipid particle of any of claims 1-93, wherein the paramyxovirus F protein is an F0 precursor or is a proteolytically cleaved form thereof comprising F1 and F2 subunits.

95. The lipid particle of claim 94, wherein the proteolytically cleaved form is a cathepsin L cleavage product.

96. The lipid particle of any of claims 1-95, wherein the paramyxovirus envelope attachment protein and the first targeting moiety and second targeting moiety are linked via one or more linkers.

97. The lipid particle of claim 96, wherein the one or more linkers are one or more peptide linker.

98. The lipid particle of any of claims 1-97, wherein the retargeted attachment protein comprises in order: paramyxovirus attachment protein-first linker-first targeting moiety-second linker-second targeting moiety.

99. The lipid particle of claim 98, wherein the first linker and/or second linker is independently a peptide linker.

100. The lipid particle of claim 99, wherein the first linker and second linker are the same.

101. The lipid particle of claim 99, wherein the first linker and second linker are different.

102. The lipid particle of claim 97 or claim 99, wherein the peptide linker is 2 to 65 amino acids in length.

103. The lipid particle of any of claims 99-102, wherein the peptide linker is a flexible linker that comprises GS, GGS, GGGGS, GGGGGS or combinations thereof.

104. The lipid particle of any of claims 99-103, wherein the peptide linker is selected from:
(GGS)n, wherein n is 1 to 10; (GGGGS)n, wherein n is 1 to 10; or (GGGGGS)n, wherein n is 1 to 6.

105. The lipid particle of any of claims 1-104, wherein the peptide linker is selected from SEQ ID NOs: 589-592.

106. The lipid particle of any of claims 1-105, wherein the lipid particle further comprises one or more additional paramyxovirus envelope attachment glycoproteins embedded in the lipid bilayer.

107. The lipid particle of claim 106, wherein the one or more additional paramyxovirus envelope attachment glycoproteins is a retargeted attachment protein comprising a paramyxovirus envelope attachment protein and a further targeting moiety.

108. The lipid particle of any of claims 1-107, wherein the at least one paramyxovirus fusion (F) protein exhibits fusogenic activity with a target cell upon binding of at least one paramyxovirus envelope attachment protein to the target molecule on the target cell.

109. The lipid particle of any of claims 1-108, wherein the lipid particle comprises a viral nucleic acid.

110. The lipid particle of claim 109, wherein the viral nucleic acid comprises one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3).

111. The lipid particle of any of claims 1-110, wherein the lipid particle is a viral vector.

112. The lipid particle of any of claims 1-111, that is a retroviral vector.

113. The lipid particle of any of claims 1-111, that is a lentiviral vector.

114. The lipid particle of any of claims 1-108 and 111-113, wherein the lipid particle is devoid of viral genomic nucleic acid.

115. The lipid particle of any of claims 1-108 and 111-113, wherein the lipid particle is devoid of viral genomic DNA.

116. The lipid particle of any of claims 1-110 and 115, that is a viral-like particle.

117. The lipid particle of any of claims 1-110, 115 and 116 that is a retroviral-like particle.

118. The lipid particle of any of claims 1-110, 115 and 116 that is a lentiviral-like particle.

119. The lipid particle of any of claims 1-118, wherein the lipid particle is produced as a preparation with increased titer compared to a reference lipid particle preparation that is similarly produced but with only the first retargeted attachment protein.

120. The lipid particle of any of claims 5-119, wherein the lipid particle is produced as a preparation with increased titer compared to a reference lipid particle preparation that is similarly produced but without the second paramyxovirus envelope attachment protein that is a variant paramyxovirus envelope attachment protein comprising one or more substitutions to reduce the native tropism relative to the wild-type paramyxovirus envelope attachment protein not comprising the one or more substitutions.

121. The lipid particle of claim 120, wherein the second paramyxovirus envelope attachment protein that is a variant paramyxovirus envelope attachment protein comprises one or more amino acid substitutions corresponding to amino acid substitutions selected from the group consisting of E501A, W504A, Q530A and E533A with reference to numbering set forth in SEQ ID NO:1.

122. The lipid particle of any of claims 1-119, wherein the lipid particle is produced in suspension culture as a preparation with increased titer compared to a reference lipid particle preparation that is similarly produced but with only the first retargeted attachment protein.

123. The lipid particle of any of claims 120-122, wherein the titer is increased by at or greater than 1.2-fold, 1.3-fold, 1.4-fold, 1.5-fold, 1.6-fold, 1.7-fold, 1.8-fold, 2-fold, 2.5-fold, 3-fold, 4-fold, 5-fold, 6-fold, 7-fold, 8-fold, 9-fold, 10-fold, or more.

124. The lipid particle of any of claims 1-123, further comprising an exogenous agent for delivery to a target cell.

125. The lipid particle of claim 124, wherein the exogenous agent is present in the lumen.

126. The lipid particle of claim 124 or 125, wherein the exogenous agent is a protein or a nucleic acid, optionally wherein the nucleic acid is a DNA or RNA.

127. The lipid particle of any of claims 124-126, wherein the exogenous agent is a nucleic acid encoding a cargo for delivery to the target cell.

128. The lipid particle of any of claims 124-127, wherein the exogenous agent is or encodes a therapeutic agent, a diagnostic agent, or a genome-modifying enzyme.

129. The lipid particle of any of claims 124-128, wherein the exogenous agent encodes a membrane protein, optionally wherein the membrane protein is an antigen receptor for targeting cells expressed by or associated with a disease or condition.

130. The lipid particle of claim 129, wherein the membrane protein is a chimeric antigen receptor (CAR).

131. The lipid particle of any of claims 124-128, wherein the exogenous agent is a nucleic acid comprising a payload gene for correcting a genetic deficiency, optionally a genetic deficiency in the target cell, optionally wherein the genetic deficiency is associated with a liver cell or a hepatocyte.

132. The lipid particle of any of claims 124-131, wherein binding of the paramyxovirus envelope attachment protein or biologically active portion thereof to a target molecule expressed on the surface of a target cell mediates fusion of the particle with the target cell and delivery of the exogenous agent to the target cell.

133. The lipid particle of any of claims 124-132, wherein at or greater than 10%, 20%, 30%, 40%, 50%, 60% of the target cells are delivered the exogenous agent.

134. The lipid particle of any of claims 124-133, wherein delivery of the exogenous cell to the target cell is increased compared to a reference particle preparation that is similarly produced but with only a first retargeted attachment protein.

135. The lipid particle of claim 134, wherein the delivery to the target cell is increased by at or greater than 1.2-fold, 1.3-fold, 1.4-fold, 1.5-fold, 1.6-fold, 1.7-fold, 1.8-fold, 2-fold, 2.5-fold, 3-fold, 4-fold, 5-fold, 6-fold, 7-fold, 8-fold, 9-fold, 10-fold, or more.

136. A producer cell comprising (a) a nucleic acid encoding a retargeted attachment protein comprising a paramyxovirus envelope attachment protein linked to (i) a first targeting moiety directed to a first target molecule expressed on the surface of a target cell, and (ii) a second targeting moiety directed to a second target molecule expressed on the surface of a target cell; and
(b) a nucleic acid encoding at least one paramyxovirus fusion (F) protein

137. The producer cell of claim 136, further comprising a nucleic acid encoding a second paramyxovirus envelope attachment protein that is a variant paramyxovirus envelope attachment protein comprising one or more amino acid substitutions to reduce the native tropism relative to the wild-type paramyxovirus envelope attachment protein not comprising the one or more amino acid substitutions.

138. The producer cell of claim 136 or 137, wherein the cell further comprises a viral nucleic acid(s).

139. The producer cell of any of claim 138, wherein the viral nucleic acid(s) are lentiviral nucleic acids.

140. The producer cell of any of claims 136-139, wherein the cell is a mammalian cell.

141. The producer cell of any of claims 136-140, wherein the producer cell is selected from the group consisting of CHO cells, BHK cells, MDCK cells, C3H 10T½ cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.

142. The producer cell of any of claims 136-141, wherein the producer cell comprises 293T cells.

143. The producer cell of any of claims 137-142, wherein the viral nucleic acid(s) lacks one or more genes involved in viral replication.

144. The producer cell of any of claims 137-143, wherein the viral nucleic acid comprises a nucleic acid encoding a viral packaging protein selected from one or more of Gag, Pol, Rev and Tat.

145. The producer cell of any of claims 138-144, wherein the viral nucleic acid comprises:
one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3).

146. A method of making a lipid particle, comprising:
a) providing a producer cell of any of claims 136-145;
b) culturing the cell under conditions that allow for production of the lipid particle, and
c) separating, enriching, or purifying the lipid particle from the cell, thereby making the lipid particle.

147. The method of claim 146, wherein the lipid particle is a pseudotyped lentiviral vector.

148. A lipid particle produced by the method of claim 146 or claim 147.

149. A composition comprising a plurality of lipid particles of any of claims 1-135 and 148.

150. The composition of claim 149, further comprising a pharmaceutically acceptable carrier.

151. A method of transducing a cell comprising contacting a cell with a lipid particle of any of claims 1-135 and 148 or a composition of claim 149 or claim 150.

152. A method of delivering an exogenous agent into a target cell, the method comprising contacting a lipid particle of any of claims 124-135 and 148 or a composition of claim 149 or claim 150 with a target cell.

153. The method of claim 151 or claim 152, wherein the contacting is in vitro or ex vivo.

154. The method of claim 151 or claim 152, wherein the contacting is in vivo in a subject.

155. A method of delivering an exogenous agent to a cell in a subject, the method comprising administering to the subject a lipid particle of any of claims 124-135 and 148 or a composition of claim 149 or claim 150.

156. The method of claim 155, wherein the exogenous agent is or encodes a therapeutic agent for treating a disease or condition in the subject.

157. A method of treatment, the method comprising administering to a subject a lipid particle of any of claims 124-135 and 148 or a composition of claim 149 or claim 150.

158. The method of any of claims 152-157, wherein the exogenous agent is or encodes a membrane protein, optionally a chimeric antigen receptor, for targeting an antigen associated with a disease or condition in the subject.

159. The method of any of claims 152-157, wherein the exogenous agent is for use in gene therapy to correct a genetic deficiency or replaces a deficient or missing gene in the subject.

160. The method of any of claims 155-159, wherein the subject is a human subject.

161. The method of any of claims 151-160, wherein the method further comprises administering to the subject one or more agents that stimulate mobilization of bone marrow cells from the bone marrow to the peripheral blood.

162. The method of any of claims 151-161, wherein the subject has previously been administered one or more agents that stimulate mobilization of bone marrow cells from the bone marrow to the peripheral blood.

163. The method of claim 161 or 162, wherein the one or more agents that stimulate mobilization are selected from the group consisting of stem cell factor (SCF), small molecule VLA-4 inhibitor BI05192, BOP (N-(benzenesulfonyl)-L-prolyl-L-0-(1-pyrrolidinylcarbonyl) tyrosine), heparin, granulocyte colony-stimulating factor (G-CSF), MGTA-145, and plerixafor (AMD3100).

164. The method of any of claims 161-163, wherein the one or more agents that stimulate mobilization comprise G-CSF.

165. The method of any of claims 161-164, wherein the one or more agents that stimulate mobilization comprise plerixafor.


SEQUENCE LISTING (770 sequences)

SEQ ID NO: 1 - protein, 602 aa, synthetic construct
  REGION 1..602: note = Uniprot Q9IH62
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 2 - protein, 557 aa, synthetic construct
  REGION 1..557: note = NiV-G backbone delCT
ILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 3 - protein, 557 aa, synthetic construct
  REGION 1..557: note = NiV-G backbone delCT and mutated (E501 A, W504A, Q530A, E533A)
ILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 4 - protein, 45 aa, synthetic construct
  REGION 1..45: note = NivG CT_45_FL
MPAENKKVRFENTTSDKGKIPSKVIKSYYGTMDIKKINEGLLDSK

SEQ ID NO: 5 - protein, 602 aa, synthetic construct
  REGION 1..602: note = NiVG with NiVG CT_45_FL, mutated (E501 A, W504A, Q530A, E533A)
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 6 - protein, 5 aa, synthetic construct
  REGION 1..5: note = NivG_CT_05 cytoplasmic tail
MLDSK

SEQ ID NO: 7 - protein, 6 aa, synthetic construct
  REGION 1..6: note = NivG_CT_06 cytoplasmic tail
MLLDSK

SEQ ID NO: 8 - protein, 7 aa, synthetic construct
  REGION 1..7: note = NivG_CT_07 cytoplasmic tail
MGLLDSK

SEQ ID NO: 9 - protein, 8 aa, synthetic construct
  REGION 1..8: note = NivG_CT_08 cytoplasmic tail
MEGLLDSK

SEQ ID NO: 10 - protein, 9 aa, synthetic construct
  REGION 1..9: note = NivG_CT_09 cytoplasmic tail
MNEGLLDSK

SEQ ID NO: 11 - protein, 10 aa, synthetic construct
  REGION 1..10: note = NivG_CT_10 cytoplasmic tail
MINEGLLDSK

SEQ ID NO: 12 - protein, 11 aa, synthetic construct
  REGION 1..11: note = NivG_CT_11 cytoplasmic tail
MKINEGLLDSK

SEQ ID NO: 13 - protein, 13 aa, synthetic construct
  REGION 1..13: note = NivG_CT_13 cytoplasmic tail
MIKKINEGLLDSK

SEQ ID NO: 14 - protein, 14 aa, synthetic construct
  REGION 1..14: note = NivG_CT_14 cytoplasmic tail
MDIKKINEGLLDSK

SEQ ID NO: 15 - protein, 15 aa, synthetic construct
  REGION 1..15: note = NivG_CT_15 cytoplasmic tail
MMDIKKINEGLLDSK

SEQ ID NO: 16 - protein, 16 aa, synthetic construct
  REGION 1..16: note = NivG_CT_16 cytoplasmic tail
MTMDIKKINEGLLDSK

SEQ ID NO: 17 - protein, 17 aa, synthetic construct
  REGION 1..17: note = NivG_CT_17 cytoplasmic tail
MGTMDIKKINEGLLDSK

SEQ ID NO: 18 - protein, 18 aa, synthetic construct
  REGION 1..18: note = NivG_CT_18 cytoplasmic tail
MYGTMDIKKINEGLLDSK

SEQ ID NO: 19 - protein, 19 aa, synthetic construct
  REGION 1..19: note = NivG_CT_19 cytoplasmic tail
MYYGTMDIKKINEGLLDSK

SEQ ID NO: 20 - protein, 20 aa, synthetic construct
  REGION 1..20: note = NivG_CT_20 cytoplasmic tail
MSYYGTMDIKKINEGLLDSK

SEQ ID NO: 21 - protein, 21 aa, synthetic construct
  REGION 1..21: note = NivG_CT_21 cytoplasmic tail
MKSYYGTMDIKKINEGLLDSK

SEQ ID NO: 22 - protein, 22 aa, synthetic construct
  REGION 1..22: note = NivG_CT_22 cytoplasmic tail
MIKSYYGTMDIKKINEGLLDSK

SEQ ID NO: 23 - protein, 23 aa, synthetic construct
  REGION 1..23: note = NivG_CT_23 cytoplasmic tail
MVIKSYYGTMDIKKINEGLLDSK

SEQ ID NO: 24 - protein, 24 aa, synthetic construct
  REGION 1..24: note = NivG_CT_24 cytoplasmic tail
MKVIKSYYGTMDIKKINEGLLDSK

SEQ ID NO: 25 - protein, 25 aa, synthetic construct
  REGION 1..25: note = NivG_CT_25 cytoplasmic tail
MSKVIKSYYGTMDIKKINEGLLDSK

SEQ ID NO: 26 - protein, 30 aa, synthetic construct
  REGION 1..30: note = NivG_CT_30 cytoplasmic tail
MKGKNPSKVIKSYYGTMDIKKINEGLLDSK

SEQ ID NO: 27 - protein, 35 aa, synthetic construct
  REGION 1..35: note = NivG_CT_35 cytoplasmic tail
MNTTSDKGKNPSKVIKSYYGTMDIKKINEGLLDSK

SEQ ID NO: 28 - protein, 40 aa, synthetic construct
  REGION 1..40: note = NivG_CT_40 cytoplasmic tail
MKVRFENTTSDKGKNPSKVIKSYYGTMDIKKINEGLLDSK

SEQ ID NO: 29 - protein, 23 aa, synthetic construct
  REGION 1..23: note = NivG_CSUR38_CT_23 cytoplasmic tail
MIIKSYYGTMDIKKINEGLLDSK

SEQ ID NO: 30 - protein, 29 aa, synthetic construct
  REGION 1..29: note = NivG_CSUR38_CT_29 cytoplasmic tail
MGKIPSKIIKSYYGTMDIKKINEGLLDSK

SEQ ID NO: 31 - protein, 35 aa, synthetic construct
  REGION 1..35: note = NivG_CSUR38_CT_35 cytoplasmic tail
MNTTSDKGKIPSKIIKSYYGTMDIKKINEGLLDSK

SEQ ID NO: 32 - protein, 42 aa, synthetic construct
  REGION 1..42: note = NivG_CSUR38_CT_42 cytoplasmic tail
MSKKVRFENTTSDKGKIPSKIIKSYYGTMDIKKINEGLLDSK

SEQ ID NO: 33 - protein, 45 aa, synthetic construct
  REGION 1..45: note = NivG_CSUR38_CT_45_FL cytoplasmic tail
MPAESKKVRFENTTSDKGKIPSKIIKSYYGTMDIKKINEGLLDSK

SEQ ID NO: 34 - protein, 27 aa, synthetic construct
  REGION 1..27: note = NivG_VRI-0626_CT_27 cytoplasmic tail
MNPSKVIKSYYGTMDIKKINEGLLDSK

SEQ ID NO: 35 - protein, 31 aa, synthetic construct
  REGION 1..31: note = NivG_VRI-0626_CT_31 cytoplasmic tail
MDKGKNPSKVIKSYYGTMDIKKINEGLLDSK

SEQ ID NO: 36 - protein, 35 aa, synthetic construct; identical to SEQ ID NO: 27
  REGION 1..35: note = NivG_VRI-0626_CT_35

SEQ ID NO: 37 - protein, 40 aa, synthetic construct; identical to SEQ ID NO: 28
  REGION 1..40: note = NivG_VRI-0626_CT_40 cytoplasmic tail

SEQ ID NO: 38 - protein, 45 aa, synthetic construct
  REGION 1..45: note = NivG_VRI-0626_CT_45_FL cytoplasmic tail
MPAENKKVRFENTTSDKGKNPSKVIKSYYGTMDIKKINEGLLDSK

SEQ ID NO: 39 - protein, 12 aa, synthetic construct
  REGION 1..12: note = NivG_CT_12 cytoplasmic tail
MKKINEGLLDSK

SEQ ID NO: 40 - protein, 8 aa, synthetic construct
  REGION 1..8: note = HevG_CT_08
MDGLLDSK

SEQ ID NO: 41 - protein, 9 aa, synthetic construct
  REGION 1..9: note = HevG_CT_09
MNDGLLDSK

SEQ ID NO: 42 - protein, 10 aa, synthetic construct
  REGION 1..10: note = HevG_CT_10
MINDGLLDSK

SEQ ID NO: 43 - protein, 11 aa, synthetic construct
  REGION 1..11: note = HevG_CT_11
MKINDGLLDSK

SEQ ID NO: 44 - protein, 12 aa, synthetic construct
  REGION 1..12: note = HevG_CT_12
MKKINDGLLDSK

SEQ ID NO: 45 - protein, 13 aa, synthetic construct
  REGION 1..13: note = HevG_CT_13
MIKKINDGLLDSK

SEQ ID NO: 46 - protein, 14 aa, synthetic construct
  REGION 1..14: note = HevG_CT_14
MDIKKINDGLLDSK

SEQ ID NO: 47 - protein, 15 aa, synthetic construct
  REGION 1..15: note = HevG_CT_15
MMDIKKINDGLLDSK

SEQ ID NO: 48 - protein, 16 aa, synthetic construct
  REGION 1..16: note = HevG_CT_16
MTMDIKKINDGLLDSK

SEQ ID NO: 49 - protein, 17 aa, synthetic construct
  REGION 1..17: note = HevG_CT_17
MGTMDIKKINDGLLDSK

SEQ ID NO: 50 - protein, 18 aa, synthetic construct
  REGION 1..18: note = HevG_CT_18
MYGTMDIKKINDGLLDSK

SEQ ID NO: 51 - protein, 19 aa, synthetic construct
  REGION 1..19: note = HevG_CT_19
MYYGTMDIKKINDGLLDSK

SEQ ID NO: 52 - protein, 20 aa, synthetic construct
  REGION 1..20: note = HevG_CT_20
MNYYGTMDIKKINDGLLDSK

SEQ ID NO: 53 - protein, 21 aa, synthetic construct
  REGION 1..21: note = HevG_CT_21
MKNYYGTMDIKKINDGLLDSK

SEQ ID NO: 54 - protein, 22 aa, synthetic construct
  REGION 1..22: note = HevG_CT_22
MIKNYYGTMDIKKINDGLLDSK

SEQ ID NO: 55 - protein, 23 aa, synthetic construct
  REGION 1..23: note = HevG_CT_23
MVIKNYYGTMDIKKINDGLLDSK

SEQ ID NO: 56 - protein, 24 aa, synthetic construct
  REGION 1..24: note = HevG_CT_24
MKVIKNYYGTMDIKKINDGLLDSK

SEQ ID NO: 57 - protein, 45 aa, synthetic construct
  REGION 1..45: note = HevG_CT_45_FL
MMADSKLVSLNNNLSGKIKDQGKVIKNYYGTMDIKKINDGLLDSK

SEQ ID NO: 58 - protein, 6 aa, synthetic construct
  REGION 1..6: note = BatPV_G_CT_6
MQNDHY

SEQ ID NO: 59 - protein, 7 aa, synthetic construct
  REGION 1..7: note = BatPV_G_CT_7
MNQNDHY

SEQ ID NO: 60 - protein, 8 aa, synthetic construct
  REGION 1..8: note = BatPV_G_CT_8
MKNQNDHY

SEQ ID NO: 61 - protein, 9 aa, synthetic construct
  REGION 1..9: note = BatPV_G_CT_9
MQKNQNDHY

SEQ ID NO: 62 - protein, 10 aa, synthetic construct
  REGION 1..10: note = BatPV_G_CT_10
MKQKNQNDHY

SEQ ID NO: 63 - protein, 11 aa, synthetic construct
  REGION 1..11: note = BatPV_G_CT_11
MKKQKNQNDHY

SEQ ID NO: 64 - protein, 12 aa, synthetic construct
  REGION 1..12: note = BatPV_G_CT_12
MWKKQKNQNDHY

SEQ ID NO: 65 - protein, 13 aa, synthetic construct
  REGION 1..13: note = BatPV_G_CT_13
MNWKKQKNQNDHY

SEQ ID NO: 66 - protein, 14 aa, synthetic construct
  REGION 1..14: note = BatPV_G_CT_14
MRNWKKQKNQNDHY

SEQ ID NO: 67 - protein, 15 aa, synthetic construct
  REGION 1..15: note = BatPV_G_CT_15
MERNWKKQKNQNDHY

SEQ ID NO: 68 - protein, 16 aa, synthetic construct
  REGION 1..16: note = BatPV_G_CT_16
MSERNWKKQKNQNDHY

SEQ ID NO: 69 - protein, 17 aa, synthetic construct
  REGION 1..17: note = BatPV_G_CT_17
MHSERNWKKQKNQNDHY

SEQ ID NO: 70 - protein, 18 aa, synthetic construct
  REGION 1..18: note = BatPV_G_CT_18
MSHSERNWKKQKNQNDHY

SEQ ID NO: 71 - protein, 19 aa, synthetic construct
  REGION 1..19: note = BatPV_G_CT_19
MGSHSERNWKKQKNQNDHY

SEQ ID NO: 72 - protein, 20 aa, synthetic construct
  REGION 1..20: note = BatPV_G_CT_20
MLGSHSERNWKKQKNQNDHY

SEQ ID NO: 73 - protein, 21 aa, synthetic construct
  REGION 1..21: note = BatPV_G_CT_21
MGLGSHSERNWKKQKNQNDHY

SEQ ID NO: 74 - protein, 22 aa, synthetic construct
  REGION 1..22: note = BatPV_G_CT_22
MFGLGSHSERNWKKQKNQNDHY

SEQ ID NO: 75 - protein, 23 aa, synthetic construct
  REGION 1..23: note = BatPV_G_CT_23
MYFGLGSHSERNWKKQKNQNDHY

SEQ ID NO: 76 - protein, 24 aa, synthetic construct
  REGION 1..24: note = BatPV_G_CT_24
MGYFGLGSHSERNWKKQKNQNDHY

SEQ ID NO: 77 - protein, 29 aa, synthetic construct
  REGION 1..29: note = BatPV_G_CT_29
MKITKQGYFGLGSHSERNWKKQKNQNDHY

SEQ ID NO: 78 - protein, 59 aa, synthetic construct
  REGION 1..59: note = BatPV_G_CT_59_FL
MPQKTVEFINMNSPLERGVSTLSDKKTLNQSKITKQGYFGLGSHSERNWKKQKNQNDHY

SEQ ID NO: 79 - protein, 6 aa, synthetic construct
  REGION 1..6: note = CedarV_G_CT_6
MLHDIK

SEQ ID NO: 80 - protein, 7 aa, synthetic construct
  REGION 1..7: note = CedarV_G_CT_7
MSLHDIK

SEQ ID NO: 81 - protein, 8 aa, synthetic construct
  REGION 1..8: note = CedarV_G_CT_8
MESLHDIK

SEQ ID NO: 82 - protein, 9 aa, synthetic construct
  REGION 1..9: note = CedarV_G_CT_9
MNESLHDIK

SEQ ID NO: 83 - protein, 10 aa, synthetic construct
  REGION 1..10: note = CedarV_G_CT_10
MLNESLHDIK

SEQ ID NO: 84 - protein, 11 aa, synthetic construct
  REGION 1..11: note = CedarV_G_CT_11
MLLNESLHDIK

SEQ ID NO: 85 - protein, 12 aa, synthetic construct
  REGION 1..12: note = CedarV_G_CT_12
MNLLNESLHDIK

SEQ ID NO: 86 - protein, 13 aa, synthetic construct
  REGION 1..13: note = CedarV_G_CT_13
MSNLLNESLHDIK

SEQ ID NO: 87 - protein, 14 aa, synthetic construct
  REGION 1..14: note = CedarV_G_CT_14
MVSNLLNESLHDIK

SEQ ID NO: 88 - protein, 15 aa, synthetic construct
  REGION 1..15: note = CedarV_G_CT_15
MNVSNLLNESLHDIK

SEQ ID NO: 89 - protein, 16 aa, synthetic construct
  REGION 1..16: note = CedarV_G_CT_16
MYNVSNLLNESLHDIK

SEQ ID NO: 90 - protein, 17 aa, synthetic construct
  REGION 1..17: note = CedarV_G_CT_17
MNYNVSNLLNESLHDIK

SEQ ID NO: 91 - protein, 18 aa, synthetic construct
  REGION 1..18: note = CedarV_G_CT_18
MKNYNVSNLLNESLHDIK

SEQ ID NO: 92 - protein, 19 aa, synthetic construct
  REGION 1..19: note = CedarV_G_CT_19
MNKNYNVSNLLNESLHDIK

SEQ ID NO: 93 - protein, 20 aa, synthetic construct
  REGION 1..20: note = CedarV_G_CT_20
MKNKNYNVSNLLNESLHDIK

SEQ ID NO: 94 - protein, 21 aa, synthetic construct
  REGION 1..21: note = CedarV_G_CT_21
MVKNKNYNVSNLLNESLHDIK

SEQ ID NO: 95 - protein, 22 aa, synthetic construct
  REGION 1..22: note = CedarV_G_CT_22
MYVKNKNYNVSNLLNESLHDIK

SEQ ID NO: 96 - protein, 23 aa, synthetic construct
  REGION 1..23: note = CedarV_G_CT_23
MYYVKNKNYNVSNLLNESLHDIK

SEQ ID NO: 97 - protein, 24 aa, synthetic construct
  REGION 1..24: note = CedarV_G_CT_24
MSYYVKNKNYNVSNLLNESLHDIK

SEQ ID NO: 98 - protein, 30 aa, synthetic construct
  REGION 1..30: note = CedarV_G_CT_30
MQKDLNKSYYVKNKNYNVSNLLNESLHDIK

SEQ ID NO: 99 - protein, 68 aa, synthetic construct
  REGION 1..68: note = CedarV_G_CT_68_FL
MLSQLQKNYLDNSNQQGDKMNNPDKKLSVNFNPLELDKGQKDLNKSYYVKNKNYNVSNLLNESLHDIK

SEQ ID NO: 100 - protein, 6 aa, synthetic construct
  REGION 1..6: note = CDV-H_CT_06
MQGGRR

SEQ ID NO: 101 - protein, 7 aa, synthetic construct
  REGION 1..7: note = CDV-H_CT_07
MEQGGRR

SEQ ID NO: 102 - protein, 8 aa, synthetic construct
  REGION 1..8: note = CDV-H_CT_08
MEEQGGRR

SEQ ID NO: 103 - protein, 9 aa, synthetic construct
  REGION 1..9: note = CDV-H_CT_09
MTEEQGGRR

SEQ ID NO: 104 - protein, 10 aa, synthetic construct
  REGION 1..10: note = CDV-H_CT_10
MVTEEQGGRR

SEQ ID NO: 105 - protein, 11 aa, synthetic construct
  REGION 1..11: note = CDV-H_CT_11
MLVTEEQGGRR

SEQ ID NO: 106 - protein, 12 aa, synthetic construct
  REGION 1..12: note = CDV-H_CT_12
MSLVTEEQGGRR

SEQ ID NO: 107 - protein, 13 aa, synthetic construct
  REGION 1..13: note = CDV-H_CT_13
MLSLVTEEQGGRR

SEQ ID NO: 108 - protein, 14 aa, synthetic construct
  REGION 1..14: note = CDV-H_CT_14
MKLSLVTEEQGGRR

SEQ ID NO: 109 - protein, 15 aa, synthetic construct
  REGION 1..15: note = CDV-H_CT_15
MSKLSLVTEEQGGRR

SEQ ID NO: 110 - protein, 16 aa, synthetic construct
  REGION 1..16: note = CDV-H_CT_16
MSSKLSLVTEEQGGRR

SEQ ID NO: 111 - protein, 17 aa, synthetic construct
  REGION 1..17: note = CDV-H_CT_17
MNSSKLSLVTEEQGGRR

SEQ ID NO: 112 - protein, 18 aa, synthetic construct
  REGION 1..18: note = CDV-H_CT_18
MANSSKLSLVTEEQGGRR

SEQ ID NO: 113 - protein, 19 aa, synthetic construct
  REGION 1..19: note = CDV-H_CT_19
MRANSSKLSLVTEEQGGRR

SEQ ID NO: 114 - protein, 20 aa, synthetic construct
  REGION 1..20: note = CDV-H_CT_20
MARANSSKLSLVTEEQGGRR

SEQ ID NO: 115 - protein, 34 aa, synthetic construct
  REGION 1..34: note = CDV-H_CT_34_FL
MLSYQDKVGAFYKDNARANSSKLSLVTEEQGGRR

SEQ ID NO: 116 - protein, 6 aa, synthetic construct
  REGION 1..6: note = HPIV2-HN_CT_06
MRIIFR

SEQ ID NO: 117 - protein, 12 aa, synthetic construct
  REGION 1..12: note = HPIV2-HN_CT_12
MIPKRTCRIIFR

SEQ ID NO: 118 - protein, 22 aa, synthetic construct
  REGION 1..22: note = HPIV2-HN_CT_22_FL
MEDYSNLSLKSIPKRTCRIIFR

SEQ ID NO: 119 - protein, 6 aa, synthetic construct
  REGION 1..6: note = MvH_CT_06
MLMIDR

SEQ ID NO: 120 - protein, 7 aa, synthetic construct
  REGION 1..7: note = MvH_CT_07
MHLMIDR

SEQ ID NO: 121 - protein, 8 aa, synthetic construct
  REGION 1..8: note = MvH_CT_08
MEHLMIDR

SEQ ID NO: 122 - protein, 9 aa, synthetic construct
  REGION 1..9: note = MvH_CT_09
MREHLMIDR

SEQ ID NO: 123 - protein, 10 aa, synthetic construct
  REGION 1..10: note = MvH_CT_10
MNREHLMIDR

SEQ ID NO: 124 - protein, 11 aa, synthetic construct
  REGION 1..11: note = MvH_CT_11
MINREHLMIDR

SEQ ID NO: 125 - protein, 12 aa, synthetic construct
  REGION 1..12: note = MvH_CT_12
MVINREHLMIDR

SEQ ID NO: 126 - protein, 13 aa, synthetic construct
  REGION 1..13: note = MvH_CT_13
MIVINREHLMIDR

SEQ ID NO: 127 - protein, 14 aa, synthetic construct
  REGION 1..14: note = MvH_CT_14
MRIVINREHLMIDR

SEQ ID NO: 128 - protein, 15 aa, synthetic construct
  REGION 1..15: note = MvH_CT_15
MSRIVINREHLMIDR

SEQ ID NO: 129 - protein, 16 aa, synthetic construct
  REGION 1..16: note = MvH_CT_16
MGSRIVINREHLMIDR

SEQ ID NO: 130 - protein, 17 aa, synthetic construct
  REGION 1..17: note = MvH_CT_17
MKGSRIVINREHLMIDR

SEQ ID NO: 131 - protein, 18 aa, synthetic construct
  REGION 1..18: note = MvH_CT_18
MPKGSRIVINREHLMIDR

SEQ ID NO: 132 - protein, 19 aa, synthetic construct
  REGION 1..19: note = MvH_CT_19
MHPKGSRIVINREHLMIDR

SEQ ID NO: 133 - protein, 20 aa, synthetic construct
  REGION 1..20: note = MvH_CT_20
MPHPKGSRIVINREHLMIDR

SEQ ID NO: 134 - protein, 34 aa, synthetic construct
  REGION 1..34: note = MvH_CT_34_FL
MSPQRDRINAFYKDNPHPKGSRIVINREHLMIDR

SEQ ID NO: 135 - protein, 6 aa, synthetic construct
  REGION 1..6: note = NDV-HN_CT_06
MRLVFR

SEQ ID NO: 136 - protein, 7 aa, synthetic construct
  REGION 1..7: note = NDV-HN_CT_07
MWRLVFR

SEQ ID NO: 137 - protein, 8 aa, synthetic construct
  REGION 1..8: note = NDV-HN_CT_08
MTWRLVFR

SEQ ID NO: 138 - protein, 9 aa, synthetic construct
  REGION 1..9: note = NDV-HN_CT_09
MNTWRLVFR

SEQ ID NO: 139 - protein, 10 aa, synthetic construct
  REGION 1..10: note = NDV-HN_CT_10
MKNTWRLVFR

SEQ ID NO: 140 - protein, 11 aa, synthetic construct
  REGION 1..11: note = NDV-HN_CT_11
MAKNTWRLVFR

SEQ ID NO: 141 - protein, 12 aa, synthetic construct
  REGION 1..12: note = NDV-HN_CT_12
MEAKNTWRLVFR

SEQ ID NO: 142 - protein, 13 aa, synthetic construct
  REGION 1..13: note = NDV-HN_CT_13
MREAKNTWRLVFR

SEQ ID NO: 143 - protein, 14 aa, synthetic construct
  REGION 1..14: note = NDV-HN_CT_14
MEREAKNTWRLVFR

SEQ ID NO: 144 - protein, 15 aa, synthetic construct
  REGION 1..15: note = NDV-HN_CT_15
MDEREAKNTWRLVFR

SEQ ID NO: 145 - protein, 16 aa, synthetic construct
  REGION 1..16: note = NDV-HN_CT_16
MNDEREAKNTWRLVFR

SEQ ID NO: 146 - protein, 17 aa, synthetic construct
  REGION 1..17: note = NDV-HN_CT_17
MENDEREAKNTWRLVFR

SEQ ID NO: 147 - protein, 18 aa, synthetic construct
  REGION 1..18: note = NDV-HN_CT_18
MLENDEREAKNTWRLVFR

SEQ ID NO: 148 - protein, 19 aa, synthetic construct
  REGION 1..19: note = NDV-HN_CT_19
MALENDEREAKNTWRLVFR

SEQ ID NO: 149 - protein, 20 aa, synthetic construct
  REGION 1..20: note = NDV-HN_CT_20
MVALENDEREAKNTWRLVFR

SEQ ID NO: 150 - protein, 26 aa, synthetic construct
  REGION 1..26: note = NDV-HN_CT_26_FL
MERGVSQVALENDEREAKNTWRLVFR

SEQ ID NO: 151 - protein, 6 aa, synthetic construct
  REGION 1..6: note = SeV_CT_06
MERSGK

SEQ ID NO: 152 - protein, 7 aa, synthetic construct
  REGION 1..7: note = SeV_CT_07
MSERSGK

SEQ ID NO: 153 - protein, 8 aa, synthetic construct
  REGION 1..8: note = SeV_CT_08
MDSERSGK

SEQ ID NO: 154 - protein, 9 aa, synthetic construct
  REGION 1..9: note = SeV_CT_09
MSDSERSGK

SEQ ID NO: 155 - protein, 10 aa, synthetic construct
  REGION 1..10: note = SeV_CT_10
MVSDSERSGK

SEQ ID NO: 156 - protein, 11 aa, synthetic construct
  REGION 1..11: note = SeV_CT_11
MLVSDSERSGK

SEQ ID NO: 157 - protein, 12 aa, synthetic construct
  REGION 1..12: note = SeV_CT_12
MKLVSDSERSGK

SEQ ID NO: 158 - protein, 13 aa, synthetic construct
  REGION 1..13: note = SeV_CT_13
MTKLVSDSERSGK

SEQ ID NO: 159 - protein, 14 aa, synthetic construct
  REGION 1..14: note = SeV_CT_14
MTTKLVSDSERSGK

SEQ ID NO: 160 - protein, 15 aa, synthetic construct
  REGION 1..15: note = SeV_CT_15
MSTTKLVSDSERSGK

SEQ ID NO: 161 - protein, 16 aa, synthetic construct
  REGION 1..16: note = SeV_CT_16
MGSTTKLVSDSERSGK

SEQ ID NO: 162 - protein, 17 aa, synthetic construct
  REGION 1..17: note = SeV_CT_17
MGGSTTKLVSDSERSGK

SEQ ID NO: 163 - protein, 18 aa, synthetic construct
  REGION 1..18: note = SeV_CT_18
MPGGSTTKLVSDSERSGK

SEQ ID NO: 164 - protein, 19 aa, synthetic construct
  REGION 1..19: note = SeV_CT_19
MSPGGSTTKLVSDSERSGK

SEQ ID NO: 165 - protein, 20 aa, synthetic construct
  REGION 1..20: note = SeV_CT_20
MTSPGGSTTKLVSDSERSGK

SEQ ID NO: 166 - protein, 32 aa, synthetic construct
  REGION 1..32: note = SeV_CT_32_FL
MDGDRSKRDSYWSTSPGGSTTKLVSDSERSGK

SEQ ID NO: 167 - protein, 35 aa, synthetic construct
  REGION 1..35: note = BaEVwt_CT
MDQAEEDTRLVQYQQTLVMAHIINLKDNIFATLRN

SEQ ID NO: 168 - protein, 28 aa, synthetic construct
  REGION 1..28: note = BaEVRLess_CT
MAHIINLKDNIFATLRNKKINEGLLDSK

SEQ ID NO: 169 - protein, 17 aa, synthetic construct
  REGION 1..17: note = BaEVRLess_CT
MAHIINLKDNIFATLRN

SEQ ID NO: 170 - protein, 24 aa, synthetic construct
  REGION 1..24: note = Cocal_CT
MKRFRSMEIDNYIRKNNSGQYRYR

SEQ ID NO: 171 - protein, 35 aa, synthetic construct
  REGION 1..35: note = Cocal_CT
MKRFRSMEIDNYIRKNNSGQYRYRKKINEGLLDSK

SEQ ID NO: 172 - protein, 6 aa, synthetic construct
  REGION 1..6: note = EboV-GP_CT
MFVFKC

SEQ ID NO: 173 - protein, 17 aa, synthetic construct
  REGION 1..17: note = EboV-GP_CT
MFVFKCKKINEGLLDSK

SEQ ID NO: 174 - protein, 43 aa, synthetic construct
  REGION 1..43: note = GaLV_CT
MLNGENELAQYKQRLVLIKVASIRDNIFQVLKKKINEGLLDSK

SEQ ID NO: 175 - protein, 32 aa, synthetic construct
  REGION 1..32: note = GaLV_CT
MLNGENELAQYKQRLVLIKVASIRDNIFQVLK

SEQ ID NO: 176 - protein, 28 aa, synthetic construct
  REGION 1..28: note = GaLV-RLess_CT
MLIKVASIRDNIFQVLKKKINEGLLDSK

SEQ ID NO: 177 - protein, 17 aa, synthetic construct
  REGION 1..17: note = GaLV-RLess_CT
MLIKVASIRDNIFQVLK

SEQ ID NO: 178 - protein, 40 aa, synthetic construct
  REGION 1..40: note = LCMV_GP_CT
MRRKWVTKVGPVKFAGCSCIGKNTLRHPKPCSGGKIHRHT

SEQ ID NO: 179 - protein, 44 aa, synthetic construct
  REGION 1..44: note = MLV-A_CT
MPEYEIPKLQHYQQTLVLAQVVSIRDKVFQVLRKKINEGLLDSK

SEQ ID NO: 180 - protein, 33 aa, synthetic construct
  REGION 1..33: note = MLV-A_CT
MPEYEIPKLQHYQQTLVLAQVVSIRDKVFQVLR

SEQ ID NO: 181 - protein, 28 aa, synthetic construct
  REGION 1..28: note = MLV-A-RLess_CT
MLAQVVSIRDKVFQVLRKKINEGLLDSK

SEQ ID NO: 182 - protein, 17 aa, synthetic construct
  REGION 1..17: note = MLV-A-RLess_CT
MLAQVVSIRDKVFQVLR

SEQ ID NO: 183 - protein, 30 aa, synthetic construct
  REGION 1..30: note = VSV-G_CT
MKGLRNMEIDTYIQRKKTHKLKICLHIGVR

SEQ ID NO: 184 - protein, 41 aa, synthetic construct
  REGION 1..41: note = VSV-G_CT
MKGLRNMEIDTYIQRKKTHKLKICLHIGVRKKINEGLLDSK

SEQ ID NO: 185 - protein, 152 aa, synthetic construct
  REGION 1..152: note = HIV-1_gp41_MLLIR-LLP1-LLP3-LLP2-gp41-CT-N
MLLIRELGQRIRRPIHRIARYAAQLVEIVRDTGEAVAIATANLLNVASNKLEQSWYQLLNWWYKLAEWGRRGLLEVIRTVILLLDRLRHYSFLCLSRLDDWILALSGNVLRISRDRDREGGEEEIGEPRDPGRPIPLHTQFSLPSYGQRVRN

SEQ ID NO: 186 - protein, 163 aa, synthetic construct
  REGION 1..163: note = HIV-1_gp41_MLLIR-LLP1-LLP3-LLP2-gp41-CT-N
MLLIRELGQRIRRPIHRIARYAAQLVEIVRDTGEAVAIATANLLNVASNKLEQSWYQLLNWWYKLAEWGRRGLLEVIRTVILLLDRLRHYSFLCLSRLDDWILALSGNVLRISRDRDREGGEEEIGEPRDPGRPIPLHTQFSLPSYGQRVRNKKINEGLLDSK

SEQ ID NO: 187 - protein, 119 aa, synthetic construct
  REGION 1..119: note = HIV-1_gp41_LLP3-LLP2-gp41-CT-N
MAVAIATANLLNVASNKLEQSWYQLLNWWYKLAEWGRRGLLEVIRTVILLLDRLRHYSFLCLSRLDDWILALSGNVLRISRDRDREGGEEEIGEPRDPGRPIPLHTQFSLPSYGQRVRN

SEQ ID NO: 188 - protein, 81 aa, synthetic construct
  REGION 1..81: note = HIV-1_gp41_LLP2-gp41-CT-N
MLLEVIRTVILLLDRLRHYSFLCLSRLDDWILALSGNVLRISRDRDREGGEEEIGEPRDPGRPIPLHTQFSLPSYGQRVRN

SEQ ID NO: 189 - protein, 48 aa, synthetic construct
  REGION 1..48: note = HIV-1_gp41_gp41-CT-N
MSGNVLRISRDRDREGGEEEIGEPRDPGRPIPLHTQFSLPSYGQRVRN

SEQ ID NO: 190 - protein, 105 aa, synthetic construct
  REGION 1..105: note = HIV-1_gp41_MLLIR-LLP1-LLP3-LLP2
MLLIRELGQRIRRPIHRIARYAAQLVEIVRDTGEAVAIATANLLNVASNKLEQSWYQLLNWWYKLAEWGRRGLLEVIRTVILLLDRLRHYSFLCLSRLDDWILAL

SEQ ID NO: 191 - protein, 101 aa, synthetic construct
  REGION 1..101: note = HIV-1_gp41_LLP1-LLP3-LLP2
MELGQRIRRPIHRIARYAAQLVEIVRDTGEAVAIATANLLNVASNKLEQSWYQLLNWWYKLAEWGRRGLLEVIRTVILLLDRLRHYSFLCLSRLDDWILAL

SEQ ID NO: 192 - protein, 72 aa, synthetic construct
  REGION 1..72: note = HIV-1_gp41_LLP3-LLP2
MAVAIATANLLNVASNKLEQSWYQLLNWWYKLAEWGRRGLLEVIRTVILLLDRLRHYSFLCLSRLDDWILAL

SEQ ID NO: 193 - protein, 34 aa, synthetic construct
  REGION 1..34: note = HIV-1_gp41_LLP2
MLLEVIRTVILLLDRLRHYSFLCLSRLDDWILAL

SEQ ID NO: 194 - protein, 68 aa, synthetic construct
  REGION 1..68: note = HIV-1_gp41_MLLIR-LLP1-LLP3
MLLIRELGQRIRRPIHRIARYAAQLVEIVRDTGEAVAIATANLLNVASNKLEQSWYQLLNWWYKLAEW

SEQ ID NO: 195 - protein, 64 aa, synthetic construct
  REGION 1..64: note = HIV-1_gp41_LLP1-LLP3
MELGQRIRRPIHRIARYAAQLVEIVRDTGEAVAIATANLLNVASNKLEQSWYQLLNWWYKLAEW

SEQ ID NO: 196 - protein, 35 aa, synthetic construct
  REGION 1..35: note = HIV-1_gp41_LLP3
MAVAIATANLLNVASNKLEQSWYQLLNWWYKLAEW

SEQ ID NO: 197 - protein, 30 aa, synthetic construct
  REGION 1..30: note = HIV-1_gp41_MLLIR-LLP1
MLLIRELGQRIRRPIHRIARYAAQLVEIVR

SEQ ID NO: 198 - protein, 26 aa, synthetic construct
  REGION 1..26: note = HIV-1_gp41_LLP1
MELGQRIRRPIHRIARYAAQLVEIVR

SEQ ID NO: 199 - protein, 5 aa, synthetic construct
  REGION 1..5: note = HIV-1_gp41_MLLIR
MLLIR

SEQ ID NO: 200 - protein, 11 aa, synthetic construct
  REGION 1..11: note = CD63_CT_1to11
MAVEGGMKCVK

SEQ ID NO: 201 - protein, 10 aa, synthetic construct
  REGION 1..10: note = CD63_CT_73to81
MCGACKENYC

SEQ ID NO: 202 - protein, 14 aa, synthetic construct
  REGION 1..14: note = CD63_CT_225to238
MVEYGSRISKVLCC

SEQ ID NO: 203 - protein, 22 aa, synthetic construct
  REGION 1..22: note = CD63_CT_1to11
MAVEGGMKCVKKKINEGLLDSK

SEQ ID NO: 204 - protein, 21 aa, synthetic construct
  REGION 1..21: note = CD63_CT_73to81
MCGACKENYCKKINEGLLDSK

SEQ ID NO: 205 - protein, 25 aa, synthetic construct
  REGION 1..25: note = CD63_CT_225to238
MVEYGSRISKVLCCKKINEGLLDSK

SEQ ID NO: 206 - protein, 48 aa, synthetic construct
  REGION 1..48: note = CD29_CT
MKGEYKPNVVTTVASKYIPNEGTDWKANMKEKEFKAFERRDHIIMLLK

SEQ ID NO: 207 - protein, 59 aa, synthetic construct
  REGION 1..59: note = CD29_CT
MKGEYKPNVVTTVASKYIPNEGTDWKANMKEKEFKAFERRDHIIMLLKKKINEGLLDSK

SEQ ID NO: 208 - protein, 569 aa, synthetic construct
  REGION 1..569: note = MvH_CT_12
MVINREHLMIDRILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 209 - protein, 573 aa, synthetic construct
  REGION 1..573: note = MvH_CT_16
MGSRIVINREHLMIDRILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 210 - protein, 577 aa, synthetic construct
  REGION 1..577: note = MvH_CT_20
MPHPKGSRIVINREHLMIDRILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 211 - protein, 576 aa, synthetic construct
  REGION 1..576: note = NivG_CT_19
MYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 212 - protein, 565 aa, synthetic construct
  REGION 1..565: note = BatPV_G_CT_8
MKNQNDHYILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 213 - protein, 565 aa, synthetic construct
  REGION 1..565: note = SeV_CT_08
MDSERSGKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 214 - protein, 574 aa, synthetic construct
  REGION 1..574: note = MLV-A-RLess_CT
MLAQVVSIRDKVFQVLRILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 215 - protein, 562 aa, synthetic construct
  REGION 1..562: note = HIV-1_gp41_MLLIR
MLLIRILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 216 - protein, 568 aa, synthetic construct
  REGION 1..568: note = CD63_CT_1to11
MAVEGGMKCVKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 217 - protein, 567 aa, synthetic construct
  REGION 1..567: note = CD63_CT_73to81
MCGACKENYCILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 218 - protein, 569 aa, synthetic construct
  REGION 1..569: note = HevG_CT_12
MKKINDGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 219 - protein, 590 aa, synthetic construct
  REGION 1..590: note = MLV-A_CT
MPEYEIPKLQHYQQTLVLAQVVSIRDKVFQVLRILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 220 - protein, 563 aa, synthetic construct
  REGION 1..563: note = NivG_CT_06
MLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 221 - protein, 570 aa, synthetic construct
  REGION 1..570: note = NivG_CT_13
MIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 222 - protein, 569 aa, synthetic construct
  REGION 1..569: note = BatPV_G_CT_12
MWKKQKNQNDHYILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 223 - protein, 563 aa, synthetic construct
  REGION 1..563: note = HPIV2-HN_CT_06
MRIIFRILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 224 - protein, 569 aa, synthetic construct
  REGION 1..569: note = HPIV2-HN_CT_12
MIPKRTCRIIFRILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 225 - protein, 569 aa, synthetic construct
  REGION 1..569: note = CD63_CT_1to11
MKLVSDSERSGKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 226 - protein, 524 aa, synthetic construct
  REGION 1..524: note = Truncated NiV fusion glycoprotein (FcDelta22) at cytoplasmic tail (with signal sequence)
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 227 - protein, 498 aa, synthetic construct
  REGION 1..498: note = Truncated mature NiV fusion glycoprotein (FcDelta22) at cytoplasmic tail
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 228 - protein, 569 aa, synthetic construct
  REGION 1..569: note = NiVG protein attachment glycoprotein Truncated and mutated (E501 A, W504A, Q530A, E533A) NiV G protein (Gc del34)
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 229 - protein, 33 aa, synthetic construct
  REGION 1..33: note = Moloney murine leukemia virus cytoplasmic tail (632-665 of UniProt P03385; R-peptide 650-665)
NRLVQFVKDRISVVQALVLTQQYHQLKPIEYEP

SEQ ID NO: 230 - protein, 5 aa, synthetic construct
  REGION 1..5: note = peptide linker
GGGGS

SEQ ID NO: 231 - protein, 6 aa, synthetic construct
  REGION 1..6: note = peptide linker
GGGGGS

SEQ ID NO: 232 - protein, 5 aa, synthetic construct
  REGION 1..5: note = peptide linker
  REPEAT 1..5: note = Repeat is 1 to 10
GGGGS

SEQ ID NO: 233 - protein, 6 aa, synthetic construct
  REGION 1..6: note = peptide linker
  REPEAT 1..6: note = Repeat is 1 to 6
GGGGGS

SEQ ID NO: 234 - protein, 546 aa, synthetic construct
  REGION 1..546: note = Hendra F
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 235 - protein, 546 aa, synthetic construct
  REGION 1..546: note = Nipah F0
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 236 - protein, 557 aa, synthetic construct
  REGION 1..557: note = CedV F
MSNKRTTVLIIISYTLFYLNNAAIVGFDFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD

SEQ ID NO: 237 - protein, 545 aa, synthetic construct
  REGION 1..545: note = Mojiang F
MALNKNMFSSLFLGYLLVYATTVQSSIHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH

SEQ ID NO: 238 - protein, 662 aa, synthetic construct
  REGION 1..662: note = Bat PV F
MKKKTDNPTISKRGHNHSRGIKSRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD

SEQ ID NO: 239 - protein, 245 aa, synthetic construct
  REGION 1..245: note = Anti-CD19 scFv (FMC63)
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 240 - protein, 242 aa, synthetic construct
  REGION 1..242: note = Anti-CD19 scFv (FMC63)
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 241 - protein, 12 aa, synthetic construct
  REGION 1..12: note = IgG4 Hinge
ESKYGPPCPPCP

SEQ ID NO: 242 - protein, 25 aa, synthetic construct
  REGION 1..25: note = CD8 Hinge
TTTPAPRPPTPAPTIASQPLSLRPE

SEQ ID NO: 243 - protein, 39 aa, synthetic construct
  REGION 1..39: note = CD28
IEVMYPPPYLDNEKSNGTIIHVKGKHLCPSPLFPGPSKP

SEQ ID NO: 244 - protein, 44 aa, synthetic construct
  REGION 1..44: note = CD8
ACRPAAGGAVHTRGLDFACDIYIWAPLAGTCGVLLLSLVITLYC

SEQ ID NO: 245 - protein, 27 aa, synthetic construct
  REGION 1..27: note = CD28
FWVLVVVGGVLACYSLLVTVAFIIFWV

SEQ ID NO: 246 - protein, 27 aa, synthetic construct
  REGION 1..27: note = CD28
FWVLVVVGGVLACYSLLVTVAFIIFWV

SEQ ID NO: 247 - protein, 41 aa, synthetic construct
  REGION 1..41: note = CD28
RSKRSRLLHSDYMNMTPRRPGPTRKHYQPYAPPRDFAAYRS

SEQ ID NO: 248 - protein, 42 aa, synthetic construct
  REGION 1..42: note = 4-1BB
KRGRKKLLYIFKQPFMRPVQTTQEEDGCSCRFPEEEEGGCEL

SEQ ID NO: 249 - protein, 112 aa, synthetic construct
  REGION 1..112: note = CD3zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 250 - protein, 112 aa, synthetic construct
  REGION 1..112: note = CD3zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 251 - skipped

SEQ ID NO: 252 - protein, 345 aa, synthetic construct
  REGION 1..345: note = P04578 (512-856) HIV -1 Env (GP41)
AVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 253 - protein, 345 aa, synthetic construct
  REGION 1..345: note = AAK08489.2 (512-856) HIV-1 NL4-3 Env (GP41)
AVGIGALFLGFLGAAGSTMGAASMTLTVQARQLLSDIVQQQNNLLRAIEAQQHLLQLTVWGIKQLQARILAVERYLKDQQLLGIWGCSGKLICTTAVPWNASWSNKSLEQIWNNMTWMEWDREINNYTSLIHSLIEESQNQQEKNEQELLELDKWASLWNWFNITNWLWYIKLFIMIVGGLVGLRIVFAVLSIVNRVRQGYSPLSFQTHLPIPRGPDRPEGIEEEGGERGRDRSIRLVNGSLALIWDDLRSLCLFSYHRLRDLLLIVTRIVELLGRRGWEALKYWWNLLQYWSQELKNSAVNLLNATAIAVAEGTDRVIEVLQAAYRAIRHIPRRIRQGLERILL

SEQ ID NO: 254 - protein, 11 aa, synthetic construct
  REGION 1..11: note = Cytoplasmic tail amino acids
KKINEGLLDSK

SEQ ID NO: 255 - protein, 520 aa, synthetic construct
  REGION 1..520: note = Hendra virus F Protein, Without signal sequence
ILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 256 - protein, 520 aa, synthetic construct
  REGION 1..520: note = Nipah virus F Protein, without signal sequence
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 257 - protein, 551 aa, synthetic construct
  REGION 1..551: note = Cedar Virus F Protein, without signal sequence
TVLIIISYTLFYLNNAAIVGFDFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD

SEQ ID NO: 258 - protein, 519 aa, synthetic construct
  REGION 1..519: note = Mojiang virus, Tongguan 1 F Protein, without signal sequence
IHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH

SEQ ID NO: 259 - protein, 640 aa, synthetic construct
  REGION 1..640: note = Bat Paramyxovirus F Protein, without signal sequence
SRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD

SEQ ID NO: 260 - protein, 8 aa, synthetic construct
  REGION 1..8: note = CDR-H1
GYTFSNYW

SEQ ID NO: 261 - protein, 8 aa, synthetic construct
  REGION 1..8: note = CDR-H2
ILPGSGST

SEQ ID NO: 262 - protein, 13 aa, synthetic construct
  REGION 1..13: note = CDR-H3
ARRGYGYDEGFDY

SEQ ID NO: 263 - protein, 6 aa, synthetic construct
  REGION 1..6: note = CDR-L1
QDINSY

SEQ ID NO: 264 - skipped

SEQ ID NO: 265 - protein, 9 aa, synthetic construct
  REGION 1..9: note = CDR-L3
LQYDEFPPT

SEQ ID NO: 266 - protein, 5 aa, synthetic construct
  REGION 1..5: note = CDR-H1
NYWIE

SEQ ID NO: 267 - protein, 17 aa, synthetic construct
  REGION 1..17: note = CDR-H2
EILPGSGSTSYNEKFKG

SEQ ID NO: 268 - protein, 11 aa, synthetic construct
  REGION 1..11: note = CDR-H3
RGYGYDEGFDY

SEQ ID NO: 269 - protein, 11 aa, synthetic construct
  REGION 1..11: note = CDR-L1
KASQDINSYLS

SEQ ID NO: 270 - protein, 7 aa, synthetic construct
  REGION 1..7: note = CDR-L2
RANRLVD

SEQ ID NO: 271 - protein, 7 aa, synthetic construct
  REGION 1..7: note = CDR-H1
GYTFSNY

SEQ ID NO: 272 - protein, 6 aa, synthetic construct
  REGION 1..6: note = CDR-H2
LPGSGS

SEQ ID NO: 273 - protein, 120 aa, synthetic construct
  REGION 1..120: note = VH
QVQLQQSGAELMKPGASVKMSCKATGYTFSNYWIEWVKQRPGHGLEWIGEILPGSGSTSYNEKFKGKATFTADTSSSTAYMQLSSLTSEDSAVYYCARRGYGYDEGFDYWGQGSTLTVSS

SEQ ID NO: 274 - protein, 108 aa, synthetic construct
  REGION 1..108: note = VL
DIKMTQSPSSMYASLGERVTITCKASQDINSYLSWFQQKPGKSPKTLIYRANRLVDGVPSRFSGSGSGQDYSLTISSLEYEDMGIYYCLQYDEFPPTFGAGTKLELKR

SEQ ID NO: 275 - protein, 15 aa, synthetic construct
  REGION 1..15: note = linker
GGGGSGGGGSGGGGS

SEQ ID NO: 276 - protein, 243 aa, synthetic construct
  REGION 1..243: note = scFv
QVQLQQSGAELMKPGASVKMSCKATGYTFSNYWIEWVKQRPGHGLEWIGEILPGSGSTSYNEKFKGKATFTADTSSSTAYMQLSSLTSEDSAVYYCARRGYGYDEGFDYWGQGSTLTVSSGGGGSGGGGSGGGGSDIKMTQSPSSMYASLGERVTITCKASQDINSYLSWFQQKPGKSPKTLIYRANRLVDGVPSRFSGSGSGQDYSLTISSLEYEDMGIYYCLQYDEFPPTFGAGTKLELKR

SEQ ID NO: 277 - protein, 8 aa, synthetic construct
  REGION 1..8: note = CDR-H1
GYTFTDYV

SEQ ID NO: 278 - protein, 8 aa, synthetic construct
  REGION 1..8: note = CDR-H2
IYPGSGSS

SEQ ID NO: 279 - protein, 10 aa, synthetic construct
  REGION 1..10: note = CDR-H3
ARPGDLGFAY

SEQ ID NO: 280 - protein, 10 aa, synthetic construct
  REGION 1..10: note = CDR-L1
QSVDYDGDSY

SEQ ID NO: 281 - skipped

SEQ ID NO: 282 - protein, 9 aa, synthetic construct
  REGION 1..9: note = CDR-L3
QQSNKDPFT

SEQ ID NO: 283 - protein, 5 aa, synthetic construct
  REGION 1..5: note = CDR-H1
DYVIS

SEQ ID NO: 284 - protein, 17 aa, synthetic construct
  REGION 1..17: note = CDR-H2
EIYPGSGSSYYNEKFKG

SEQ ID NO: 285 - protein, 8 aa, synthetic construct
  REGION 1..8: note = CDR-H3
PGDLGFAY

SEQ ID NO: 286 - protein, 15 aa, synthetic construct
  REGION 1..15: note = CDR-L1
KASQSVDYDGDSYMN

SEQ ID NO: 287 - protein, 7 aa, synthetic construct
  REGION 1..7: note = CDR-L2
AASNLES

SEQ ID NO: 288 - protein, 9 aa, synthetic construct
  REGION 1..9: note = CDR-L3
QQSNKDPFT

SEQ ID NO: 289 - protein, 7 aa, synthetic construct
  REGION 1..7: note = CDR-H1
GYTFTDY

SEQ ID NO: 290 - protein, 6 aa, synthetic construct
  REGION 1..6: note = CDR-H2
YPGSGS

SEQ ID NO: 291 - protein, 117 aa, synthetic construct
  REGION 1..117: note = VH
QVQLQQSGPELVKPGASVKMSCKASGYTFTDYVISWVRQAPGQGLEWIGEIYPGSGSSYYNEKFKGRATLTADKSSNTAYMQLSSLRSEDSAVYFCARPGDLGFAYWGQGTLVTVSS

SEQ ID NO: 292 - protein, 112 aa, synthetic construct
  REGION 1..112: note = VL
DIVLTQSPSSLAVSLGQRATISCKASQSVDYDGDSYMNWYQQKPGQPPKLLIYAASNLESGIPARFSGSGSGTDFTLTIHPVEEEDAATYYCQQSNKDPFTFGGGTKLELKR

SEQ ID NO: 293 - protein, 244 aa, synthetic construct
  REGION 1..244: note = scFv
QVQLQQSGPELVKPGASVKMSCKASGYTFTDYVISWVRQAPGQGLEWIGEIYPGSGSSYYNEKFKGRATLTADKSSNTAYMQLSSLRSEDSAVYFCARPGDLGFAYWGQGTLVTVSSGGGGSGGGGSGGGGSDIVLTQSPSSLAVSLGQRATISCKASQSVDYDGDSYMNWYQQKPGQPPKLLIYAASNLESGIPARFSGSGSGTDFTLTIHPVEEEDAATYYCQQSNKDPFTFGGGTKLELKR

SEQ ID NO: 294 - protein, 8 aa, synthetic construct
  REGION 1..8: note = CDR-H1
GGTFSSYA

SEQ ID NO: 295 - protein, 8 aa, synthetic construct
  REGION 1..8: note = CDR-H2
INPNSGGT

SEQ ID NO: 296 - protein, 11 aa, synthetic construct
  REGION 1..11: note = CDR-H3
ARDGYSGSYSD

SEQ ID NO: 297 - protein, 12 aa, synthetic construct
  REGION 1..12: note = CDR-L1
QSVLSSSYNKNY

SEQ ID NO: 298 - skipped

SEQ ID NO: 299 - protein, 9 aa, synthetic construct
  REGION 1..9: note = CDR-L3
QQYYSTPWT

SEQ ID NO: 300 - protein, 5 aa, synthetic construct
  REGION 1..5: note = CDR-H1
SYAIS

SEQ ID NO: 301 - protein, 17 aa, synthetic construct
  REGION 1..17: note = CDR-H2
WINPNSGGTNYAQKFQG

SEQ ID NO: 302 - protein, 9 aa, synthetic construct
  REGION 1..9: note = CDR-H3
DGYSGSYSD

SEQ ID NO: 303 - protein, 17 aa, synthetic construct
  REGION 1..17: note = CDR-L1
KSSQSVLSSSYNKNYLA

SEQ ID NO: 304 - protein, 7 aa, synthetic construct
  REGION 1..7: note = CDR-L2
WASTRES

SEQ ID NO: 305 - protein, 7 aa, synthetic construct
  REGION 1..7: note = CDR-H1
GGTFSSY

SEQ ID NO: 306 - protein, 6 aa, synthetic construct
  REGION 1..6: note = CDR-H2
NPNSGG

SEQ ID NO: 307 - protein, 118 aa, synthetic construct
  REGION 1..118: note = VH
QVQLVQSGAEVKKPGASVKVSCKASGGTFSSYAISWVRQAPGQGLEWMGWINPNSGGTNYAQKFQGRVTMTRDTSTSTVYMELSSLRSEDTAVYYCARDGYSGSYSDWGQGTLVTVSS

SEQ ID NO: 308 - protein, 113 aa, synthetic construct
  REGION 1..113: note = VL
DIVMTQSPDSLAVSLGERATINCKSSQSVLSSSYNKNYLAWYQQKPGQPPKLLIYWASTRESGVPDRFSGSGSGTDFTLTISSLQAEDVAVYYCQQYYSTPWTFGQGTKVEIK

SEQ ID NO: 309 - protein, 246 aa, synthetic construct
  REGION 1..246: note = scFv
QVQLVQSGAEVKKPGASVKVSCKASGGTFSSYAISWVRQAPGQGLEWMGWINPNSGGTNYAQKFQGRVTMTRDTSTSTVYMELSSLRSEDTAVYYCARDGYSGSYSDWGQGTLVTVSSGGGGSGGGGSGGGGSDIVMTQSPDSLAVSLGERATINCKSSQSVLSSSYNKNYLAWYQQKPGQPPKLLIYWASTRESGVPDRFSGSGSGTDFTLTISSLQAEDVAVYYCQQYYSTPWTFGQGTKVEIK

SEQ ID NO: 310 - protein, 8 aa, synthetic construct
  REGION 1..8: note = CDR-H1
GYTFTSYD

SEQ ID NO: 311 - protein, 8 aa, synthetic construct
  REGION 1..8: note = CDR-H2
IIPLSGAP

SEQ ID NO: 312 - protein, 15 aa, synthetic construct
  REGION 1..15: note = CDR-H3
ARGALYNWNDGWFDP

SEQ ID NO: 313 - protein, 6 aa, synthetic construct
  REGION 1..6: note = CDR-L1
QDIGDY

SEQ ID NO: 314 - skipped

SEQ ID NO: 315 - protein, 9 aa, synthetic construct
  REGION 1..9: note = CDR-L3
QQANSFPLT

SEQ ID NO: 316 - protein, 5 aa, synthetic construct
  REGION 1..5: note = CDR-H1
SYDIN

SEQ ID NO: 317 - protein, 17 aa, synthetic construct
  REGION 1..17: note = CDR-H2
GIIPLSGAPNYAHKFQG

SEQ ID NO: 318 - protein, 13 aa, synthetic construct
  REGION 1..13: note = CDR-H3
GALYNWNDGWFDP

SEQ ID NO: 319 - protein, 11 aa, synthetic construct
  REGION 1..11: note = CDR-L1
RASQDIGDYLA

SEQ ID NO: 320 - protein, 7 aa, synthetic construct
  REGION 1..7: note = CDR-L2
DASSLQS

SEQ ID NO: 321 - protein, 7 aa, synthetic construct
  REGION 1..7: note = CDR-H1
GYTFTSY

SEQ ID NO: 322 - protein, 6 aa, synthetic construct
  REGION 1..6: note = CDR-H2
IPLSGA

SEQ ID NO: 323 - protein, 122 aa, synthetic construct
  REGION 1..122: note = VH
QVQLVQSGAEVKKPGSSVKVSCKASGYTFTSYDINWVRQAPGQGLEWMGGIIPLSGAPNYAHKFQGRVTITADESTSTAYMELSSLRSEDTAVYYCARGALYNWNDGWFDPWGQGTLVTVSS

SEQ ID NO: 324 - protein, 107 aa, synthetic construct
  REGION 1..107: note = VL
DIQMTQSPSSLSASVGDRVTITCRASQDIGDYLAWYQQKPGKAPKLLIYDASSLQSGVPSRFSGSGSGTDFTLTISSLQPEDFATYYCQQANSFPLTFGGGTKVEIK

SEQ ID NO: 325 - protein, 244 aa, synthetic construct
  REGION 1..244: note = scFv
QVQLVQSGAEVKKPGSSVKVSCKASGYTFTSYDINWVRQAPGQGLEWMGGIIPLSGAPNYAHKFQGRVTITADESTSTAYMELSSLRSEDTAVYYCARGALYNWNDGWFDPWGQGTLVTVSSGGGGSGGGGSGGGGSDIQMTQSPSSLSASVGDRVTITCRASQDIGDYLAWYQQKPGKAPKLLIYDASSLQSGVPSRFSGSGSGTDFTLTISSLQPEDFATYYCQQANSFPLTFGGGTKVEIK

SEQ ID NO: 326 - protein, 8 aa, synthetic construct
  REGION 1..8: note = CDR-H1
GDTFSDHA

SEQ ID NO: 327 - protein, 8 aa, synthetic construct
  REGION 1..8: note = CDR-H2
MNPKIGNT

SEQ ID NO: 328 - protein, 12 aa, synthetic construct
  REGION 1..12: note = CDR-H3
VYDSSGYDAFDI

SEQ ID NO: 329 - protein, 12 aa, synthetic construct
  REGION 1..12: note = CDR-L1
QSVLSTSYNRNF

SEQ ID NO: 330 - skipped

SEQ ID NO: 331 - protein, 9 aa, synthetic construct
  REGION 1..9: note = CDR-L3
QQYYSTPYT

SEQ ID NO: 332 - protein, 5 aa, synthetic construct
  REGION 1..5: note = CDR-H1
DHAIN

SEQ ID NO: 333 - protein, 17 aa, synthetic construct
  REGION 1..17: note = CDR-H2
WMNPKIGNTGYAQKFQG

SEQ ID NO: 334 - protein, 10 aa, synthetic construct
  REGION 1..10: note = CDR-H3
DSSGYDAFDI

SEQ ID NO: 335 - protein, 17 aa, synthetic construct
  REGION 1..17: note = CDR-L1
KSSQSVLSTSYNRNFLA

SEQ ID NO: 336 - protein, 7 aa, synthetic construct
  REGION 1..7: note = CDR-L2
WASTRQS

SEQ ID NO: 337 - protein, 7 aa, synthetic construct
  REGION 1..7: note = CDR-H1
GDTFSDH

SEQ ID NO: 338 - protein, 6 aa, synthetic construct
  REGION 1..6: note = CDR-H2
NPKIGN

SEQ ID NO: 339 - protein, 119 aa, synthetic construct
  REGION 1..119: note = VH
QVQLVQSGAEVKKPGASVKVSCKASGDTFSDHAINWVRQAPGQGLEWMGWMNPKIGNTGYAQKFQGRVTMTRDTSTSTVYMELSSLRSEDTAVYYCVYDSSGYDAFDIWGQGTTVTVSS

SEQ ID NO: 340 - protein, 113 aa, synthetic construct
  REGION 1..113: note = VL
DIVMTQSPDSLAVSLGERATINCKSSQSVLSTSYNRNFLAWYQQKPGQPPKLLIYWASTRQSGVPDRFSGSGSGTDFTLTISSLQAEDVAVYYCQQYYSTPYTFGQGTKLEIK

SEQ ID NO: 341 - protein, 247 aa, synthetic construct
  REGION 1..247: note = scFv
QVQLVQSGAEVKKPGASVKVSCKASGDTFSDHAINWVRQAPGQGLEWMGWMNPKIGNTGYAQKFQGRVTMTRDTSTSTVYMELSSLRSEDTAVYYCVYDSSGYDAFDIWGQGTTVTVSSGGGGSGGGGSGGGGSDIVMTQSPDSLAVSLGERATINCKSSQSVLSTSYNRNFLAWYQQKPGQPPKLLIYWASTRQSGVPDRFSGSGSGTDFTLTISSLQAEDVAVYYCQQYYSTPYTFGQGTKLEIK

SEQ ID NO: 342 - protein, 8 aa, synthetic construct
  REGION 1..8: note = CDR-H1
GYSLITHW

SEQ ID NO: 343 - protein, 8 aa, synthetic construct
  REGION 1..8: note = CDR-H2
INPSDGVT

SEQ ID NO: 344 - protein, 11 aa, synthetic construct
  REGION 1..11: note = CDR-H3
AREYYGEGFDY

SEQ ID NO: 345 - protein, 6 aa, synthetic construct
  REGION 1..6: note = CDR-L1
QGISNY

SEQ ID NO: 346 - skipped

SEQ ID NO: 347 - protein, 9 aa, synthetic construct
  REGION 1..9: note = CDR-L3
QQSYSTPLT

SEQ ID NO: 348 - protein, 5 aa, synthetic construct
  REGION 1..5: note = CDR-H1
THWMH

SEQ ID NO: 349 - protein, 17 aa, synthetic construct
  REGION 1..17: note = CDR-H2
MINPSDGVTYYAQTFQG

SEQ ID NO: 350 - protein, 9 aa, synthetic construct
  REGION 1..9: note = CDR-H3
EYYGEGFDY

SEQ ID NO: 351 - protein, 11 aa, synthetic construct
  REGION 1..11: note = CDR-L1
RASQGISNYLA

SEQ ID NO: 352 - protein, 7 aa, synthetic construct
  REGION 1..7: note = CDR-L2
SASNLQS

SEQ ID NO: 353 - protein, 7 aa, synthetic construct
  REGION 1..7: note = CDR-H1
GYSLITH

SEQ ID NO: 354 - protein, 6 aa, synthetic construct
  REGION 1..6: note = CDR-H2
NPSDGV

SEQ ID NO: 355 - protein, 118 aa, synthetic construct
  REGION 1..118: note = VH
QVQLVQSGAEVKKPGASVKVSCKASGYSLITHWMHWVRQAPGQGLEWMGMINPSDGVTYYAQTFQGRVTMTRDTSTSTVYMELSSLRSEDTAVYYCAREYYGEGFDYWGQGTLVTVSS

SEQ ID NO: 356 - protein, 108 aa, synthetic construct
  REGION 1..108: note = VL
DIQMTQSPSSLSASVGDRVTITCRASQGISNYLAWYQQKPGKAPKLLIYSASNLQSGVPSRFSGSGSGTDFTLTISSLQPEDFATYYCQQSYSTPLTFGGGTKVEIKR

SEQ ID NO: 357 - protein, 241 aa, synthetic construct
  REGION 1..241: note = scFv
QVQLVQSGAEVKKPGASVKVSCKASGYSLITHWMHWVRQAPGQGLEWMGMINPSDGVTYYAQTFQGRVTMTRDTSTSTVYMELSSLRSEDTAVYYCAREYYGEGFDYWGQGTLVTVSSGGGGSGGGGSGGGGSDIQMTQSPSSLSASVGDRVTITCRASQGISNYLAWYQQKPGKAPKLLIYSASNLQSGVPSRFSGSGSGTDFTLTISSLQPEDFATYYCQQSYSTPLTFGGGTKVEIKR

SEQ ID NO: 358 - protein, 8 aa, synthetic construct
  REGION 1..8: note = CDR-H1
GFTFSGYW

SEQ ID NO: 359 - protein, 8 aa, synthetic construct
  REGION 1..8: note = CDR-H2
ISPGGGST

SEQ ID NO: 360 - protein, 14 aa, synthetic construct
  REGION 1..14: note = CDR-H3
ASSLTATHTYEYDY

SEQ ID NO: 361 - protein, 5 aa, synthetic construct
  REGION 1..5: note = CDR-H1
GYWMY

SEQ ID NO: 362 - protein, 17 aa, synthetic construct
  REGION 1..17: note = CDR-H2
AISPGGGSTYYPDSVKG

SEQ ID NO: 363 - protein, 12 aa, synthetic construct
  REGION 1..12: note = CDR-H3
SLTATHTYEYDY

SEQ ID NO: 364 - protein, 7 aa, synthetic construct
  REGION 1..7: note = CDR-H1
GFTFSGY

SEQ ID NO: 365 - protein, 6 aa, synthetic construct
  REGION 1..6: note = CDR-H2
SPGGGS

SEQ ID NO: 366 - protein, 121 aa, synthetic construct
  REGION 1..121: note = VHH
EVQLVESGGGLVQSGGSLRLSCAASGFTFSGYWMYWVRQAPGKGLEWVSAISPGGGSTYYPDSVKGRFTISRDNAKNTLYLQMNSLEPEDTALYYCASSLTATHTYEYDYWGQGTQVTVSS

SEQ ID NO: 367 - protein, 45 aa, synthetic construct
  REGION 1..45: note = CD8a hinge domain
TTTPAPRPPTPAPTIASQPLSLRPEACRPAAGGAVHTRGLDFACD

SEQ ID NO: 368 - protein, 24 aa, synthetic construct
  REGION 1..24: note = CD8a transmembrane domain
IYIWAPLAGTCGVLLLSLVITLYC

SEQ ID NO: 369 - protein, 122 aa, synthetic construct
  REGION 1..122: note = CD8_1 VH
QVQLVQSGAEVKKPGASVKVSCKASGGTFSSYAISWVRQAPGQGLEWMGIIDPSDGNTNYAQNFQGRVTMTRDTSTSTVYMELSSLRSEDTAVYYCAKERAAAGYYYYMDVWGQGTTVTVSS

SEQ ID NO: 370 - protein, 108 aa, synthetic construct
  REGION 1..108: note = CD8_1 VL
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 371 - protein, 119 aa, synthetic construct
  REGION 1..119: note = CD8_2 VH
QVQLVQSGAEVKKPGASVKVSCKASGYTFTDYYIQWVRQAPGQGLEWMGWINPNSGGTSYAQKFQGRVTMTRDTSTSTVYMELSSLRSEDTAVYYCAKEGDYYYGMDAWGQGTMVTVSS

SEQ ID NO: 372 - protein, 113 aa, synthetic construct
  REGION 1..113: note = CD8_2 VL
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLQTPHTFGQGTKVEIKR

SEQ ID NO: 373 - protein, 117 aa, synthetic construct
  REGION 1..117: note = CD8_3 VH
QVQLVQSGAEVKKPGASVKVSCKASGYTFTSYYMHWVRQAPGQGLEWMGGFDPEDGETIYAQKFQGRVTMTRDTSTSTVYMELSSLRSEDTAVYYCARDQGWGMDVWGQGTTVTVSS

SEQ ID NO: 374 - protein, 108 aa, synthetic construct
  REGION 1..108: note = CD8_3 VL
DIQMTQSPSSLSASVGDRVTITCRASQSISSYLNWYQQKPGKAPKLLIYAASSLQSGVPSRFSGSGSGTDFTLTISSLQPEDFATYYCQQTYSTPYTFGQGTKLEIKR

SEQ ID NO: 375 - protein, 118 aa, synthetic construct
  REGION 1..118: note = CD8_4 VH
QVQLVQSGAEVKKPGASVKVSCKASGYTFTNHYMHWVRQAPGQGLEWMGWMNPNSGNTGYAQKFQGRVTMTRDTSTSTVYMELSSLRSEDTAVYYCASSESGSDLDYWGQGTLVTVSS

SEQ ID NO: 376 - protein, 108 aa, synthetic construct
  REGION 1..108: note = CD8_4 VL
DIQMTQSPSSLSASVGDRVTITCRASQTIGNYVNWYQQKPGKAPKLLIYGASNLHTGVPSRFSGSGSGTDFTLTISSLQPEDFATYYCQQTYSAPLTFGGGTKVEIKR

SEQ ID NO: 377 - protein, 125 aa, synthetic construct
  REGION 1..125: note = VHH
QVQLVESGGGLVQAGGSLRLSCAASGRTFSGYVMGWFRQAPGKQRKFVAAISRGGLSTSYADSVKGRFTISRDNAKNTVFLQMNTLKPEDTAVYYCAADRSDLYEITAASNIDSWGQGTLVTVSS

SEQ ID NO: 378 - protein, 21 aa, synthetic construct
  REGION 1..21: note = CD8a signal peptide
MALPVTALLLPLALLLHAARP

SEQ ID NO: 379 - protein, 20 aa, synthetic construct
  REGION 1..20: note = IgK signal peptide
METDTLLLWVLLLWVPGSTG

SEQ ID NO: 380 - protein, 22 aa, synthetic construct
  REGION 1..22: note = GMCSFR-a (CSF2RA) signal peptide
MLLLVTSLLLCELPHPAFLLIP

SEQ ID NO: 381 - protein, 45 aa, synthetic construct; identical to SEQ ID NO: 367
  REGION 1..45: note = CD8a hinge domain

SEQ ID NO: 382 - protein, 39 aa, synthetic construct; identical to SEQ ID NO: 243
  REGION 1..39: note = CD28 hinge domain

SEQ ID NO: 383 - protein, 42 aa, synthetic construct
  REGION 1..42: note = CD28 hinge domain
AAAIEVMYPPPYLDNEKSNGTIIHVKGKHLCPSPLFPGPSKP

SEQ ID NO: 384 - protein, 12 aa, synthetic construct
  REGION 1..12: note = IgG4 hinge domain
ESKYGPPCPPCP

SEQ ID NO: 385 - protein, 12 aa, synthetic construct
  REGION 1..12: note = IgG4 hinge domain
ESKYGPPCPSCP

SEQ ID NO: 386 - protein, 229 aa, synthetic construct
  REGION 1..229: note = IgG4 hinge-CH2-CH3 domain
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 387 - protein, 24 aa, synthetic construct
  REGION 1..24: note = CD8a transmembrane domain
IYIWAPLAGTCGVLLLSLVITLYC

SEQ ID NO: 388 - protein, 27 aa, synthetic construct
  REGION 1..27: note = CD28 transmembrane domain
FWVLVVVGGVLACYSLLVTVAFIIFWV

SEQ ID NO: 389 - protein, 28 aa, synthetic construct
  REGION 1..28: note = CD28 transmembrane domain
MFWVLVVVGGVLACYSLLVTVAFIIFWV

SEQ ID NO: 390 - protein, 42 aa, synthetic construct; identical to SEQ ID NO: 248
  REGION 1..42: note = 4-1BB costimulatory domain

SEQ ID NO: 391 - protein, 41 aa, synthetic construct; identical to SEQ ID NO: 247
  REGION 1..41: note = CD28 costimulatory domain

SEQ ID NO: 392 - protein, 112 aa, synthetic construct; identical to SEQ ID NO: 249
  REGION 1..112: note = CD3z signaling domain

SEQ ID NO: 393 - protein, 112 aa, synthetic construct; identical to SEQ ID NO: 250
  REGION 1..112: note = CD3z signaling domain (with Q to K mutation at position 14)

SEQ ID NO: 394 - protein, 245 aa, synthetic construct; identical to SEQ ID NO: 239
  REGION 1..245: note = Anti-CD19 FMC63 scFv entire sequence, with Whitlow linker

SEQ ID NO: 395 - protein, 107 aa, synthetic construct
  REGION 1..107: note = Anti-CD19 FMC63 scFv light chain variable region
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 396 - protein, 6 aa, synthetic construct
  REGION 1..6: note = Anti-CD19 FMC63 scFv light chain CDR1
QDISKY

SEQ ID NO: 397 - skipped

SEQ ID NO: 398 - protein, 9 aa, synthetic construct
  REGION 1..9: note = Anti-CD19 FMC63 scFv light chain CDR3
QQGNTLPYT

SEQ ID NO: 399 - protein, 18 aa, synthetic construct
  REGION 1..18: note = Whitlow linker
GSTSGSGKPGSGEGSTKG

SEQ ID NO: 400 - protein, 120 aa, synthetic construct
  REGION 1..20: note = Anti-CD19 FMC63 scFv heavy chain variable region
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 401 - protein, 8 aa, synthetic construct
  REGION 1..8: note = Anti-CD19 FMC63 scFv heavy chain CDR1
GVSLPDYG

SEQ ID NO: 402 - protein, 7 aa, synthetic construct
  REGION 1..7: note = Anti-CD19 FMC63 scFv heavy chain CDR2
IWGSETT

SEQ ID NO: 403 - protein, 14 aa, synthetic construct
  REGION 1..14: note = Anti-CD19 FMC63 scFv heavy chain CDR3
AKHYYYGGSYAMDY

SEQ ID NO: 404 - protein, 242 aa, synthetic construct; identical to SEQ ID NO: 240
  REGION 1..242: note = Anti-CD19 FMC63 scFv entire sequence, with 3xG4S linker

SEQ ID NO: 405 - protein, 15 aa, synthetic construct
  REGION 1..15: note = 3xG4S linker
GGGGSGGGGSGGGGS

SEQ ID NO: 406 - DNA, 1467 bp, synthetic construct
  source 1..1467: mol_type = other DNA
  misc_feature 1..1467: note = Exemplary CD19 CAR nucleotide sequence
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 407 - protein, 489 aa, synthetic construct
  REGION 1..489: note = Exemplary CD19 CAR amino acid sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 408 - DNA, 1458 bp, synthetic construct
  source 1..1458: mol_type = other DNA
  misc_feature 1..1458: note = Tisagenlecleucel CD19 CAR nucleotide sequence
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 409 - protein, 486 aa, synthetic construct
  REGION 1..486: note = Tisagenlecleucel CD19 CAR amino acid sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 410 - DNA, 1383 bp, synthetic construct
  source 1..1383: mol_type = other DNA
  misc_feature 1..1383: note = Lisocabtagene maraleucel CD19 CAR nucleotide sequence
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 411 - protein, 461 aa, synthetic construct
  REGION 1..461: note = Lisocabtagene maraleucel CD19 CAR amino acid sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 412 - DNA, 1467 bp, synthetic construct
  source 1..1467: mol_type = other DNA
  misc_feature 1..1467: note = Axicabtagene ciloleucel CD19 CAR nucleotide sequence
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 413 - protein, 489 aa, synthetic construct
  REGION 1..489: note = Axicabtagene ciloleucel CD19 CAR amino acid sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 414 - protein, 246 aa, synthetic construct
  REGION 1..246: note = Anti-CD20 Leu16 scFv entire sequence, with Whitlow linker
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGSGKPGSGEGSTKGEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 415 - protein, 106 aa, synthetic construct
  REGION 1..106: note = Anti-CD20 Leu16 scFv light chain variable region
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 416 - protein, 10 aa, synthetic construct
  REGION 1..10: note = Anti-CD20 Leu16 scFv light chain CDR1
RASSSVNYMD

SEQ ID NO: 417 - protein, 7 aa, synthetic construct
  REGION 1..7: note = Anti-CD20 Leu16 scFv light chain CDR2
ATSNLAS

SEQ ID NO: 418 - protein, 9 aa, synthetic construct
  REGION 1..9: note = Anti-CD20 Leu16 scFv light chain CDR3
QQWSFNPPT

SEQ ID NO: 419 - protein, 122 aa, synthetic construct
  REGION 1..122: note = Anti-CD20 Leu16 scFv heavy chain
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 420 - protein, 5 aa, synthetic construct
  REGION 1..5: note = Anti-CD20 Leu16 scFv heavy chain CDR1
SYNMH

SEQ ID NO: 421 - protein, 17 aa, synthetic construct
  REGION 1..17: note = Anti-CD20 Leu16 scFv heavy chain CDR2
AIYPGNGDTSYNQKFKG

SEQ ID NO: 422 - protein, 13 aa, synthetic construct
  REGION 1..13: note = Anti-CD20 Leu16 scFv heavy chain CDR3
SNYYGSSYWFFDV

SEQ ID NO: 423 - protein, 246 aa, synthetic construct
  REGION 1..246: note = Anti-CD22 m971 scFv entire sequence, with 3xG4S linker
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 424 - protein, 124 aa, synthetic construct
  REGION 1..124: note = Anti-CD22 m971 scFv heavy chain variable region
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSS

SEQ ID NO: 425 - protein, 10 aa, synthetic construct
  REGION 1..10: note = Anti-CD22 m971 scFv heavy chain CDR1
GDSVSSNSAA

SEQ ID NO: 426 - protein, 9 aa, synthetic construct
  REGION 1..9: note = Anti-CD22 m971 scFv heavy chain CDR2
TYYRSKWYN

SEQ ID NO: 427 - protein, 14 aa, synthetic construct
  REGION 1..14: note = Anti-CD22 m971 scFv heavy chain CDR3
AREVTGDLEDAFDI

SEQ ID NO: 428 - protein, 107 aa, synthetic construct
  REGION 1..107: note = Anti-CD22 m971 scFv light chain
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 429 - protein, 6 aa, synthetic construct
  REGION 1..6: note = Anti-CD22 m971 scFv light chain CDR1
QTIWSY

SEQ ID NO: 430 - skipped

SEQ ID NO: 431 - protein, 9 aa, synthetic construct
  REGION 1..9: note = Anti-CD22 m971 scFv light chain CDR3
QQSYSIPQT

SEQ ID NO: 432 - protein, 246 aa, synthetic construct
  REGION 1..246: note = Anti-CD22 m971-L7 scFv entire sequence, with 3xG4S linker
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 433 - protein, 124 aa, synthetic construct
  REGION 1..124: note = Anti-CD22 m971-L7 scFv heavy chain variable region
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSS

SEQ ID NO: 434 - protein, 10 aa, synthetic construct
  REGION 1..10: note = Anti-CD22 m971-L7 scFv heavy chain CDR1
GDSVSSNSVA

SEQ ID NO: 435 - protein, 9 aa, synthetic construct
  REGION 1..9: note = Anti-CD22 m971-L7 scFv heavy chain CDR2
TYYRSTWYN

SEQ ID NO: 436 - protein, 14 aa, synthetic construct
  REGION 1..14: note = Anti-CD22 m971-L7 scFv heavy chain CDR3
AREVTGDLEDAFDI

SEQ ID NO: 437 - protein, 107 aa, synthetic construct
  REGION 1..107: note = Anti-CD22 m971-L7 scFv light chain variable region
DIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 438 - protein, 6 aa, synthetic construct
  REGION 1..6: note = Anti-CD22 m971-L7 scFv light chain CDR1
QTIWSY

SEQ ID NO: 439 - skipped

SEQ ID NO: 440 - protein, 9 aa, synthetic construct
  REGION 1..9: note = Anti-CD22 m971-L7 scFv light chain CDR3
QQSYSIPQT

SEQ ID NO: 441 - protein, 246 aa, synthetic construct
  REGION 1..246: note = Anti-BCMA C11D5.3 scFv entire sequence, with Whitlow linker
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 442 - protein, 111 aa, synthetic construct
  REGION 1..111: note = Anti-BCMA C11D5.3 scFv light chain variable region
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 443 - protein, 15 aa, synthetic construct
  REGION 1..15: note = Anti-BCMA C11D5.3 scFv light chain CDR1
RASESVSVIGAHLIH

SEQ ID NO: 444 - protein, 7 aa, synthetic construct
  REGION 1..7: note = Anti-BCMA C11D5.3 scFv light chain CDR2
LASNLET

SEQ ID NO: 445 - protein, 9 aa, synthetic construct
  REGION 1..9: note = Anti-BCMA C11D5.3 scFv light chain CDR3
LQSRIFPRT

SEQ ID NO: 446 - protein, 117 aa, synthetic construct
  REGION 1..117: note = Anti-BCMA C11D5.3 scFv heavy chain variable region
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 447 - protein, 5 aa, synthetic construct
  REGION 1..5: note = Anti-BCMA C11D5.3 scFv heavy chain CDR1
DYSIN

SEQ ID NO: 448 - protein, 17 aa, synthetic construct
  REGION 1..17: note = Anti-BCMA C11D5.3 scFv heavy chain CDR2
WINTETREPAYAYDFRG

SEQ ID NO: 449 - protein, 8 aa, synthetic construct
  REGION 1..8: note = Anti-BCMA C11D5.3 scFv heavy chain CDR3
DYSYAMDY

SEQ ID NO: 450 - protein, 246 aa, synthetic construct
  REGION 1..246: note = Anti-BCMA C12A3.2 scFv entire sequence, with Whitlow linker
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 451 - protein, 111 aa, synthetic construct
  REGION 1..111: note = Anti-BCMA C12A3.2 scFv light chain variable region
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 452 - protein, 15 aa, synthetic construct
  REGION 1..15: note = Anti-BCMA C12A3.2 scFv light chain CDR1
RASESVTILGSHLIY

SEQ ID NO: 453 - protein, 7 aa, synthetic construct
  REGION 1..7: note = Anti-BCMA C12A3.2 scFv light chain CDR2
LASNVQT

SEQ ID NO: 454 - protein, 9 aa, synthetic construct
  REGION 1..9: note = Anti-BCMA C12A3.2 scFv light chain CDR3
LQSRTIPRT

SEQ ID NO: 455 - protein, 117 aa, synthetic construct
  REGION 1..117: note = Anti-BCMA C12A3.2 scFv heavy chain variable region
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 456 - protein, 5 aa, synthetic construct
  REGION 1..5: note = Anti-BCMA C12A3.2 scFv heavy chain CDR1
HYSMN

SEQ ID NO: 457 - protein, 17 aa, synthetic construct
  REGION 1..17: note = Anti-BCMA C12A3.2 scFv heavy chain CDR2
RINTESGVPIYADDFKG

SEQ ID NO: 458 - protein, 8 aa, synthetic construct
  REGION 1..8: note = Anti-BCMA C12A3.2 scFv heavy chain CDR3
DYLYSLDF

SEQ ID NO: 459 - protein, 121 aa, synthetic construct
  REGION 1..121: note = Anti-BCMA FHVH33 entire sequence
EVQLLESGGGLVQPGGSLRLSCAASGFTFSSYAMSWVRQAPGKGLEWVSSISGSGDYIYYADSVKGRFTISRDISKNTLYLQMNSLRAEDTAVYYCAKEGTGANSSLADYRGQGTLVTVSS

SEQ ID NO: 460 - protein, 8 aa, synthetic construct
  REGION 1..8: note = Anti-BCMA FHVH33 CDR1
GFTFSSYA

SEQ ID NO: 461 - protein, 8 aa, synthetic construct
  REGION 1..8: note = Anti-BCMA FHVH33 CDR2
ISGSGDYI

SEQ ID NO: 462 - protein, 14 aa, synthetic construct
  REGION 1..14: note = Anti-BCMA FHVH33 CDR3
AKEGTGANSSLADY

SEQ ID NO: 463 - protein, 243 aa, synthetic construct
  REGION 1..243: note = Anti-BCMA CT103A scFv entire sequence, with Whitlow linker
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 464 - protein, 106 aa, synthetic construct
  REGION 1..106: note = Anti-BCMA CT103A scFv light chain variable region
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIK

SEQ ID NO: 465 - protein, 6 aa, synthetic construct
  REGION 1..6: note = Anti-BCMA CT103A scFv light chain CDR1
QSISSY

SEQ ID NO: 466 - skipped

SEQ ID NO: 467 - protein, 8 aa, synthetic construct
  REGION 1..8: note = Anti-BCMA CT103A scFv light chain CDR3
QQKYDLLT

SEQ ID NO: 468 - protein, 119 aa, synthetic construct
  REGION 1..119: note = Anti-BCMA CT103A scFv heavy chain variable region
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 469 - protein, 10 aa, synthetic construct
  REGION 1..10: note = Anti-BCMA CT103A scFv heavy chain CDR1
GGSISSSSYY

SEQ ID NO: 470 - protein, 7 aa, synthetic construct
  REGION 1..7: note = Anti-BCMA CT103A scFv heavy chain CDR2
ISYSGST

SEQ ID NO: 471 - protein, 11 aa, synthetic construct
  REGION 1..11: note = Anti-BCMA CT103A scFv heavy chain CDR3
ARDRGDTILDV

SEQ ID NO: 472 - DNA, 1503 bp, synthetic construct
  source 1..1503: mol_type = other DNA
  misc_feature 1..1503: note = Exemplary BCMA CAR nucleotide sequence
atggccttaccagtgaccgccttgctcctgccgctggccttgctgctccacgccgccaggccggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcaaaaatacgacctcctcacttttggcggagggaccaaggttgagatcaaaggcagcaccagcggctccggcaagcctggctctggcgagggcagcacaaagggacagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctcctatagtgggagcacctactacaacccgtccctcaagagtcgagtcaccatatccgtagacacgtccaagaaccagttctccctgaagctgagttctgtgaccgccgcagacacggcggtgtactactgcgccagagatcgtggagacaccatactagacgtatggggtcagggtacaatggtcaccgtcagctcattcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccaga

SEQ ID NO: 473 - protein, 501 aa, synthetic construct
  REGION 1..501: note = Exemplary BCMA CAR amino acid sequence
MALPVTALLLPLALLLHAARPDIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 474 - protein, 5 aa, synthetic construct
  REGION 1..5: note = CD3 CDR-H1
RYTMH

SEQ ID NO: 475 - protein, 17 aa, synthetic construct
  REGION 1..17: note = CD3 CDR-H2
YINPSRGYTNYNQKFKD

SEQ ID NO: 476 - protein, 10 aa, synthetic construct
  REGION 1..10: note = CD3 CDR-H3
YYDDHYCLDY

SEQ ID NO: 477 - protein, 10 aa, synthetic construct
  REGION 1..10: note = CD3 CDR-L1
RASSSVSYMN

SEQ ID NO: 478 - protein, 7 aa, synthetic construct
  REGION 1..7: note = CD3 CDR-L2
DTSKVAS

SEQ ID NO: 479 - protein, 9 aa, synthetic construct
  REGION 1..9: note = CD3 CDR-L3
QQWSSNPLT

SEQ ID NO: 480 - protein, 121 aa, synthetic construct
  REGION 1..121: note = CD3 VH
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVE

SEQ ID NO: 481 - protein, 106 aa, synthetic construct
  REGION 1..106: note = CD3 VL
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 482 - protein, 243 aa, synthetic construct
  REGION 1..243: note = CD3 scFv
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 483 - protein, 5 aa, synthetic construct
  REGION 1..5: note = CDR-H1
SYAIS

SEQ ID NO: 484 - protein, 17 aa, synthetic construct
  REGION 1..17: note = CDR-H2
IIDPSDGNTNYAQNFQG

SEQ ID NO: 485 - protein, 13 aa, synthetic construct
  REGION 1..13: note = CDR-H3
ERAAAGYYYYMDV

SEQ ID NO: 486 - protein, 11 aa, synthetic construct
  REGION 1..11: note = CDR-L1
RASQSISSYLN

SEQ ID NO: 487 - protein, 7 aa, synthetic construct
  REGION 1..7: note = CDR-L2
AASSLQS

SEQ ID NO: 488 - protein, 9 aa, synthetic construct
  REGION 1..9: note = CDR-L3
QQSYSTPLT

SEQ ID NO: 489 - protein, 245 aa, synthetic construct
  REGION 1..245: note = CD8 scFv
QVQLVQSGAEVKKPGASVKVSCKASGGTFSSYAISWVRQAPGQGLEWMGIIDPSDGNTNYAQNFQGRVTMTRDTSTSTVYMELSSLRSEDTAVYYCAKERAAAGYYYYMDVWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 490 - protein, 5 aa, synthetic construct
  REGION 1..5: note = CDR-H1
DYYIQ

SEQ ID NO: 491 - protein, 17 aa, synthetic construct
  REGION 1..17: note = CDR-H2
WINPNSGGTSYAQKFQG

SEQ ID NO: 492 - protein, 10 aa, synthetic construct
  REGION 1..10: note = CDR-H3
EGDYYYGMDA

SEQ ID NO: 493 - protein, 16 aa, synthetic construct
  REGION 1..16: note = CDR-L1
RSSQSLLHSNGYNYLD

SEQ ID NO: 494 - protein, 7 aa, synthetic construct
  REGION 1..7: note = CDR-L2
LGSNRAS

SEQ ID NO: 495 - protein, 9 aa, synthetic construct
  REGION 1..9: note = CDR-L3
MQGLQTPHT

SEQ ID NO: 496 - protein, 247 aa, synthetic construct
  REGION 1..247: note = CD8 scFv
QVQLVQSGAEVKKPGASVKVSCKASGYTFTDYYIQWVRQAPGQGLEWMGWINPNSGGTSYAQKFQGRVTMTRDTSTSTVYMELSSLRSEDTAVYYCAKEGDYYYGMDAWGQGTMVTVSSGGGGSGGGGSGGGGSDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLQTPHTFGQGTKVEIKR

SEQ ID NO: 497 - protein, 5 aa, synthetic construct
  REGION 1..5: note = CDR-H1
SYYMH

SEQ ID NO: 498 - protein, 17 aa, synthetic construct
  REGION 1..17: note = CDR-H2
GFDPEDGETIYAQKFQG

SEQ ID NO: 499 - protein, 8 aa, synthetic construct
  REGION 1..8: note = CDR-H3
DQGWGMDV

SEQ ID NO: 500 - protein, 9 aa, synthetic construct
  REGION 1..9: note = CDR-L3
QQTYSTPYT

SEQ ID NO: 501 - protein, 240 aa, synthetic construct
  REGION 1..240: note = CD8 scFv
QVQLVQSGAEVKKPGASVKVSCKASGYTFTSYYMHWVRQAPGQGLEWMGGFDPEDGETIYAQKFQGRVTMTRDTSTSTVYMELSSLRSEDTAVYYCARDQGWGMDVWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQTYSTPYTFGQGTKLEIKR

SEQ ID NO: 502 - protein, 5 aa, synthetic construct
  REGION 1..5: note = CDR-H1
NHYMH

SEQ ID NO: 503 - protein, 17 aa, synthetic construct
  REGION 1..17: note = CDR-H2
WMNPNSGNTGYAQKFQG

SEQ ID NO: 504 - protein, 9 aa, synthetic construct
  REGION 1..9: note = CDR-H3
SESGSDLDY

SEQ ID NO: 505 - protein, 11 aa, synthetic construct
  REGION 1..11: note = CDR-L1
RASQTIGNYVN

SEQ ID NO: 506 - protein, 7 aa, synthetic construct
  REGION 1..7: note = CDR-L2
GASNLHT

SEQ ID NO: 507 - protein, 9 aa, synthetic construct
  REGION 1..9: note = CDR-L3
QQTYSAPLT

SEQ ID NO: 508 - protein, 241 aa, synthetic construct
  REGION 1..241: note = CD8 scFv
QVQLVQSGAEVKKPGASVKVSCKASGYTFTNHYMHWVRQAPGQGLEWMGWMNPNSGNTGYAQKFQGRVTMTRDTSTSTVYMELSSLRSEDTAVYYCASSESGSDLDYWGQGTLVTVSSGGGGSGGGGSGGGGSDIQMTQSPSSLSASVGDRVTITCRASQTIGNYVNWYQQKPGKAPKLLIYGASNLHTGVPSRFSGSGSGTDFTLTISSLQPEDFATYYCQQTYSAPLTFGGGTKVEIKR

SEQ ID NO: 509 - protein, 5 aa, synthetic construct
  REGION 1..5: note = CDR-H1
GYVMG

SEQ ID NO: 510 - protein, 17 aa, synthetic construct
  REGION 1..17: note = CDR-H2
AISRGGLSTSYADSVKG

SEQ ID NO: 511 - protein, 16 aa, synthetic construct
  REGION 1..16: note = CDR-H3
DRSDLYEITAASNIDS

SEQ ID NO: 512 - protein, 127 aa, synthetic construct
  REGION 1..127: note = CD117_B1 VHH
QVQLVESGGGLVQAGGSLRLSCAASGRTNSNYAMGWFRQAPGKEREFVSVIGRGLGSTYYADSVKGRFTISRDNAKNTVYLQMNSLKPEDAAVYYCAADLEFVFDEMGWIPGEHAYWGRGTLVTVSS

SEQ ID NO: 513 - protein, 125 aa, synthetic construct
  REGION 1..125: note = CD117_B2 VHH
QVQLVESGGRLVQAGESLRLSCSASGGTFSSYLMAWFRQVPGKEREIVAFITWHGHTTYYTDSVKGRFAISRDNARNTMYLQMNSLRPEDTAVYYCAADQRQEFSGTYSREYNYWGQGTQVTVSS

SEQ ID NO: 514 - protein, 127 aa, synthetic construct
  REGION 1..127: note = CD117_B3 VHH
QVQLVESGGGLVQPGGSLRLSCAASGQTFTDYHHHVAWFRQAPGRERDFVAVISWSTGNTDYADSQRARFTISKDNAKNTVYLQMNNLKFEDTAVYYCAARDYHPRFSYSADDYNYWGQGTLVTVSS

SEQ ID NO: 515 - protein, 121 aa, synthetic construct
  REGION 1..121: note = CD117_B4 VHH
EVQLVESGGGLVQAGGSLRLSCAASGSSINEMGYYRQAPGKERAPRELVATITSGGSTNYADSVKGRFTISRDNAKNTVYLQMNSLKPEDTAVYYCAALRIMTMGRPYDYWGQGTQVTVSS

SEQ ID NO: 516 - protein, 246 aa, synthetic construct
  REGION 1..246: note = CD133 scFv (b3)
EIQLQQSGPDLMKPGASVKISCKASGYSFTNYYVHWVKQSLDKSLEWIGYVDPFNGDFNYNQKFKDKATLTVDKSSSTAYMHLSSLTSEDSAVYYCARGGLDWYDTSYWYFDVWGAGTAVTVSSGGGGSGGGGSGGGGSNIVMTQSPKSMSMSLGERVTLSCKASENVDTYVSWYQQKPEQSPKVLIYGASNRYTGVPDRFTGSGSATDFSLTISNVQAEDLADYHCGQSYRYPLTFGAGTKLELK

SEQ ID NO: 517 - protein, 124 aa, synthetic construct
  REGION 1..124: note = CD133 VH (b3)
EIQLQQSGPDLMKPGASVKISCKASGYSFTNYYVHWVKQSLDKSLEWIGYVDPFNGDFNYNQKFKDKATLTVDKSSSTAYMHLSSLTSEDSAVYYCARGGLDWYDTSYWYFDVWGAGTAVTVSS

SEQ ID NO: 518 - protein, 5 aa, synthetic construct
  REGION 1..5: note = CD133 CDR-H1 (b3) (Kabat)
NYYVH

SEQ ID NO: 519 - protein, 17 aa, synthetic construct
  REGION 1..17: note = CD133 CDR-H2 (b3) (Kabat)
YVDPFNGDFNYNQKFKD

SEQ ID NO: 520 - protein, 15 aa, synthetic construct
  REGION 1..15: note = CD133 CDR-H3 (b3) (Kabat, Chothia)
GGLDWYDTSYWYFDV

SEQ ID NO: 521 - protein, 107 aa, synthetic construct
  REGION 1..107: note = CD133 VL (b3)
NIVMTQSPKSMSMSLGERVTLSCKASENVDTYVSWYQQKPEQSPKVLIYGASNRYTGVPDRFTGSGSATDFSLTISNVQAEDLADYHCGQSYRYPLTFGAGTKLELK

SEQ ID NO: 522 - protein, 11 aa, synthetic construct
  REGION 1..11: note = CD133 CDR-L1 (b3) (Kabat, Chothia)
KASENVDTYVS

SEQ ID NO: 523 - protein, 7 aa, synthetic construct
  REGION 1..7: note = CD133 CDR-L2 (b3) (Kabat)
GASNRYT

SEQ ID NO: 524 - protein, 9 aa, synthetic construct
  REGION 1..9: note = CD133 CDR-L3 (b3) (Kabat)
GQSYRYPLT

SEQ ID NO: 525 - protein, 247 aa, synthetic construct
  REGION 1..247: note = CD133 scFv (b4)
EVQLVESGGGLVQPGGSLRLSCAASGFNIYYYGSMHWVRQAPGKGLEWVASISPYYGSTYYADSVKGRFTISADTSKNTAYLQMNSLRAEDTAVYYCARHASSGYGHYAVYGIDYWGQGTLVTVSSGGGGSGGGGSGGGGSDIQMTQSPSSLSASVGDRVTITCRASQGSSYVAWYQQKPGKAPKLLIYSASYLYSGVPSRFSGSRSGTDFTLTISSLQPEDFATYYCQQGVWSLITFGQGTKVEIK

SEQ ID NO: 526 - protein, 126 aa, synthetic construct
  REGION 1..126: note = CD133 VH (b4)
EVQLVESGGGLVQPGGSLRLSCAASGFNIYYYGSMHWVRQAPGKGLEWVASISPYYGSTYYADSVKGRFTISADTSKNTAYLQMNSLRAEDTAVYYCARHASSGYGHYAVYGIDYWGQGTLVTVSS

SEQ ID NO: 527 - protein, 6 aa, synthetic construct
  REGION 1..6: note = CD133 CDR-H1 (b4) (Kabat)
YYGSMH

SEQ ID NO: 528 - protein, 17 aa, synthetic construct
  REGION 1..17: note = CD133 CDR-H2 (b4) (Kabat)
SISPYYGSTYYADSVKG

SEQ ID NO: 529 - protein, 16 aa, synthetic construct
  REGION 1..16: note = CD133 CDR-H3 (b4) (Kabat, Chothia)
HASSGYGHYAVYGIDY

SEQ ID NO: 530 - protein, 106 aa, synthetic construct
  REGION 1..106: note = CD133 VL (b4)
DIQMTQSPSSLSASVGDRVTITCRASQGSSYVAWYQQKPGKAPKLLIYSASYLYSGVPSRFSGSRSGTDFTLTISSLQPEDFATYYCQQGVWSLITFGQGTKVEIK

SEQ ID NO: 531 - protein, 10 aa, synthetic construct
  REGION 1..10: note = CD133 CDR-L1 (b4) (Kabat, Chothia)
RASQGSSYVA

SEQ ID NO: 532 - protein, 7 aa, synthetic construct
  REGION 1..7: note = CD133 CDR-L1 (b4) (Kabat, Chothia)
SASYLYS

SEQ ID NO: 533 - protein, 9 aa, synthetic construct
  REGION 1..9: note = CD133 CDR-L1 (b4) (Kabat, Chothia)
QQGVWSLIT

SEQ ID NO: 534 - protein, 238 aa, synthetic construct
  REGION 1..238: note = CD133 scFv (b1)
EVKLVESGPELVKPGETVKISCKASGYTFTDYSMHWVRQAPGKGLEWMGWINTETGEPSYADDFKGRFAFSLDTSASTAYLQINSLRSEDTATYFCATDYGDYFDYWGQGTTVTVSSGGGGSGGGGSGGGGSDIVLSQSPAIMSASPGEKVTISCSASSSVSYMYWYQQKPGSSPKPWIYRTSNLASGVPARFSGSGSGTSYSLTISSMEPEDAATYYCQQYHSYPPTFGAGTKLELK

SEQ ID NO: 535 - protein, 117 aa, synthetic construct
  REGION 1..117: note = CD133 VH (b1)
EVKLVESGPELVKPGETVKISCKASGYTFTDYSMHWVRQAPGKGLEWMGWINTETGEPSYADDFKGRFAFSLDTSASTAYLQINSLRSEDTATYFCATDYGDYFDYWGQGTTVTVSS

SEQ ID NO: 536 - protein, 5 aa, synthetic construct
  REGION 1..5: note = CD133 CDR-H1 (b1) (Kabat)
DYSMH

SEQ ID NO: 537 - protein, 17 aa, synthetic construct
  REGION 1..17: note = CD133 CDR-H2 (b1) (Kabat)
WINTETGEPSYADDFKG

SEQ ID NO: 538 - protein, 8 aa, synthetic construct
  REGION 1..8: note = CD133 CDR-H3 (b1) (Kabat, Chothia)
DYGDYFDY

SEQ ID NO: 539 - protein, 106 aa, synthetic construct
  REGION 1..106: note = CD133 VL (b1)
DIVLSQSPAIMSASPGEKVTISCSASSSVSYMYWYQQKPGSSPKPWIYRTSNLASGVPARFSGSGSGTSYSLTISSMEPEDAATYYCQQYHSYPPTFGAGTKLELK

SEQ ID NO: 540 - protein, 10 aa, synthetic construct
  REGION 1..10: note = CD133 CDR-L1 (b1) (Kabat, Chothia)
SASSSVSYMY

SEQ ID NO: 541 - protein, 7 aa, synthetic construct
  REGION 1..7: note = CD133 CDR-L2 (b1) (Kabat, Chothia)
RTSNLAS

SEQ ID NO: 542 - protein, 9 aa, synthetic construct
  REGION 1..9: note = CD133 CDR-L3 (b1) (Kabat, Chothia)
QQYHSYPPT

SEQ ID NO: 543 - protein, 245 aa, synthetic construct
  REGION 1..245: note = CD133 scFv (b2)
DVQLQESGPGLVKPSQSLSLTCSVTGYSITSGYYWSWIRQFPGNKLEWMGYISYDGSNNYNPSLKNRISITRDTSKNQIFLKLNSVTTEDTATYYCTRVYFGHPYYYGMDYWGQGISVTVGGGGSGGGGSGGGGSQIVLTQSPAIMSASLGERVTMTCTASSSVSSFYLHWYRQKPGSSPKLWIYSTSNLASGVPARFSGSGSGTSYSLTISSMEAEDAATYYCHQYHRSPYMYMFGGGTKLEIK

SEQ ID NO: 544 - protein, 120 aa, synthetic construct
  REGION 1..120: note = CD133 VH (b2)
DVQLQESGPGLVKPSQSLSLTCSVTGYSITSGYYWSWIRQFPGNKLEWMGYISYDGSNNYNPSLKNRISITRDTSKNQIFLKLNSVTTEDTATYYCTRVYFGHPYYYGMDYWGQGISVTV

SEQ ID NO: 545 - protein, 6 aa, synthetic construct
  REGION 1..6: note = CD133 CDR-H1 (b2) (Kabat)
SGYYWS

SEQ ID NO: 546 - protein, 16 aa, synthetic construct
  REGION 1..16: note = CD133 CDR-H2 (b2) (Kabat)
YISYDGSNNYNPSLKN

SEQ ID NO: 547 - protein, 13 aa, synthetic construct
  REGION 1..13: note = CD133 CDR-H3 (b2) (Kabat, Chothia)
VYFGHPYYYGMDY

SEQ ID NO: 548 - protein, 110 aa, synthetic construct
  REGION 1..110: note = CD133 VL (b2)
QIVLTQSPAIMSASLGERVTMTCTASSSVSSFYLHWYRQKPGSSPKLWIYSTSNLASGVPARFSGSGSGTSYSLTISSMEAEDAATYYCHQYHRSPYMYMFGGGTKLEIK

SEQ ID NO: 549 - protein, 12 aa, synthetic construct
  REGION 1..12: note = CD133 CDR-L1 (b2) (Kabat, Chothia)
TASSSVSSFYLH

SEQ ID NO: 550 - protein, 7 aa, synthetic construct
  REGION 1..7: note = CD133 CDR-L2 (b2) (Kabat, Chothia)
STSNLAS

SEQ ID NO: 551 - protein, 11 aa, synthetic construct
  REGION 1..11: note = CD133 CDR-L3 (b2) (Kabat, Chothia)
HQYHRSPYMYM

SEQ ID NO: 552 - protein, 242 aa, synthetic construct
  REGION 1..242: note = CD133 scFv (b5)
EVQLVESGGGLVQPGGSLRLSCAASGFNLSSSSIHWVRQAPGKGLEWVAYIYPYYSYTYYADSVKGRFTISADTSKNTAYLQMNSLRAEDTAVYYCARFGSVAGFDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYSHAGHLFTFGQGTKVEIK

SEQ ID NO: 553 - protein, 118 aa, synthetic construct
  REGION 1..118: note = CD133 VH (b5)
EVQLVESGGGLVQPGGSLRLSCAASGFNLSSSSIHWVRQAPGKGLEWVAYIYPYYSYTYYADSVKGRFTISADTSKNTAYLQMNSLRAEDTAVYYCARFGSVAGFDYWGQGTLVTVSS

SEQ ID NO: 554 - protein, 5 aa, synthetic construct
  REGION 1..5: note = CD133 CDR-H1 (b5) (Kabat)
SSSIH

SEQ ID NO: 555 - protein, 17 aa, synthetic construct
  REGION 1..17: note = CD133 CDR-H2 (b5) (Kabat)
YIYPYYSYTYYADSVKG

SEQ ID NO: 556 - protein, 9 aa, synthetic construct
  REGION 1..9: note = CD133 CDR-H3 (b5) (Kabat, Chothia)
FGSVAGFDY

SEQ ID NO: 557 - protein, 109 aa, synthetic construct
  REGION 1..109: note = CD133 VL (b5)
DIQMTQSPSSLSASVGDRVTITCRASQSVSSAVAWYQQKPGKAPKLLIYSASSLYSGVPSRFSGSRSGTDFTLTISSLQPEDFATYYCQQYSHAGHLFTFGQGTKVEIK

SEQ ID NO: 558 - protein, 11 aa, synthetic construct
  REGION 1..11: note = CD133 CDR-L1 (b5) (Kabat, Chothia)
RASQSVSSAVA

SEQ ID NO: 559 - protein, 7 aa, synthetic construct
  REGION 1..7: note = CD133 CDR-L2 (b5) (Kabat, Chothia)
SASSLYS

SEQ ID NO: 560 - protein, 11 aa, synthetic construct
  REGION 1..11: note = CD133 CDR-L3 (b5) (Kabat, Chothia)
QQYSHAGHLFT

SEQ ID NO: 561 - protein, 604 aa, synthetic construct
  REGION 1..604: note = Henipavirus protein G (GENBANK ID: AF017149)
MMADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 562 - protein, 622 aa, synthetic construct
  REGION 1..622: note = Henipavirus protein G (GENBANK ID: JQ001776)
MLSQLQKNYLDNSNQQGDKMNNPDKKLSVNFNPLELDKGQKDLNKSYYVKNKNYNVSNLLNESLHDIKFCIYCIFSLLIIITIINIITISIVITRLKVHEENNGMESPNLQSIQDSLSSLTNMINTEITPRIGILVTATSVTLSSSINYVGTKTNQLVNELKDYITKSCGFKVPELKLHECNISCADPKISKSAMYSTNAYAELAGPPKIFCKSVSKDPDFRLKQIDYVIPVQQDRSICMNNPLLDISDGFFTYIHYEGINSCKKSDSFKVLLSHGEIVDRGDYRPSLYLLSSHYHPYSMQVINCVPVTCNQSSFVFCHISNNTKTLDNSDYSSDEYYITYFNGIDRPKTKKIPINNMTADNRYIHFTFSGGGGVCLGEEFIIPVTTVINTDVFTHDYCESFNCSVQTGKSLKEICSESLRSPTNSSRYNLNGIMIISQNNMTDFKIQLNGITYNKLSFGSPGRLSKTLGQVLYYQSSMSWDTYLKAGFVEKWKPFTPNWMNNTVISRPNQGNCPRYHKCPEICYGGTYNDIAPLDLGKDMYVSVILDSDQLAENPEITVFNSTTILYKERVSKDELNTRSTTTSCFLFLDEPWCISVLETNRFNGKSIRPEIYSYKIPKYC

SEQ ID NO: 563 - protein, 632 aa, synthetic construct
  REGION 1..632: note = Henipavirus protein G (GENBANK ID: NC_025256)
MPQKTVEFINMNSPLERGVSTLSDKKTLNQSKITKQGYFGLGSHSERNWKKQKNQNDHYMTVSTMILEILVVLGIMFNLIVLTMVYYQNDNINQRMAELTSNITVLNLNLNQLTNKIQREIIPRITLIDTATTITIPSAITYILATLTTRISELLPSINQKCEFKTPTLVLNDCRINCTPPLNPSDGVKMSSLATNLVAHGPSPCRNFSSVPTIYYYRIPGLYNRTALDERCILNPRLTISSTKFAYVHSEYDKNCTRGFKYYELMTFGEILEGPEKEPRMFSRSFYSPTNAVNYHSCTPIVTVNEGYFLCLECTSSDPLYKANLSNSTFHLVILRHNKDEKIVSMPSFNLSTDQEYVQIIPAEGGGTAESGNLYFPCIGRLLHKRVTHPLCKKSNCSRTDDESCLKSYYNQGSPQHQVVNCLIRIRNAQRDNPTWDVITVDLTNTYPGSRSRIFGSFSKPMLYQSSVSWHTLLQVAEITDLDKYQLDWLDTPYISRPGGSECPFGNYCPTVCWEGTYNDVYSLTPNNDLFVTVYLKSEQVAENPYFAIFSRDQILKEFPLDAWISSARTTTISCFMFNNEIWCIAALEITRLNDDIIRPIYYSFWLPTDCRTPYPHTGKMTRVPLRSTYNY

SEQ ID NO: 564 - protein, 625 aa, synthetic construct
  REGION 1..625: note = Henipavirus protein G (GENBANK ID: NC_025352)
MATNRDNTITSAEVSQEDKVKKYYGVETAEKVADSISGNKVFILMNTLLILTGAIITITLNITNLTAAKSQQNMLKIIQDDVNAKLEMFVNLDQLVKGEIKPKVSLINTAVSVSIPGQISNLQTKFLQKYVYLEESITKQCTCNPLSGIFPTSGPTYPPTDKPDDDTTDDDKVDTTIKPIEYPKPDGCNRTGDHFTMEPGANFYTVPNLGPASSNSDECYTNPSFSIGSSIYMFSQEIRKTDCTAGEILSIQIVLGRIVDKGQQGPQASPLLVWAVPNPKIINSCAVAAGDEMGWVLCSVTLTAASGEPIPHMFDGFWLYKLEPDTEVVSYRITGYAYLLDKQYDSVFIGKGGGIQKGNDLYFQMYGLSRNRQSFKALCEHGSCLGTGGGGYQVLCDRAVMSFGSEESLITNAYLKVNDLASGKPVIIGQTFPPSDSYKGSNGRMYTIGDKYGLYLAPSSWNRYLRFGITPDISVRSTTWLKSQDPIMKILSTCTNTDRDMCPEICNTRGYQDIFPLSEDSEYYTYIGITPNNGGTKNFVAVRDSDGHIASIDILQNYYSITSATISCFMYKDEIWCIAITEGKKQKDNPQRIYAHSYKIRQMCYNMKSATVTVGNAKNITIRRY

SEQ ID NO: 565 - protein, 6 aa, synthetic construct
  REGION 1..6: note = CD133 CDR-H2 (b1) (Chothia)
NTETGE

SEQ ID NO: 566 - protein, 8 aa, synthetic construct
  REGION 1..8: note = CD133 CDR-H1 (b2) (Chothia)
GYSITSGY

SEQ ID NO: 567 - protein, 5 aa, synthetic construct
  REGION 1..5: note = CD133 CDR-H2 (b2) (Chothia)
SYDGS

SEQ ID NO: 568 - protein, 7 aa, synthetic construct
  REGION 1..7: note = CD133 CDR-H1 (b3) (Chothia)
GYSFTNY

SEQ ID NO: 569 - protein, 6 aa, synthetic construct
  REGION 1..6: note = CD133 CDR-H2 (b3) (Chothia)
DPFNGD

SEQ ID NO: 570 - protein, 8 aa, synthetic construct
  REGION 1..8: note = CD133 CDR-H1 (b4) (Chothia)
GFNIYYYG

SEQ ID NO: 571 - protein, 6 aa, synthetic construct
  REGION 1..6: note = CD133 CDR-H2 (b4) (Chothia)
SPYYGS

SEQ ID NO: 572 - protein, 7 aa, synthetic construct
  REGION 1..7: note = CD133 CDR-H1 (b5) (Chothia)
GFNLSSS

SEQ ID NO: 573 - protein, 6 aa, synthetic construct
  REGION 1..6: note = CD133 CDR-H2 (b5) (Chothia)
YPYYSY

SEQ ID NO: 574 - protein, 822 aa, synthetic construct
  REGION 1..822: note = Blinded NiVG protein attachment glycoprotein + linker + CD133 scFv (b1)
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCTGGGGSGGGGSGGGGSEVKLVESGPELVKPGETVKISCKASGYTFTDYSMHWVRQAPGKGLEWMGWINTETGEPSYADDFKGRFAFSLDTSASTAYLQINSLRSEDTATYFCATDYGDYFDYWGQGTTVTVSSGGGGSGGGGSGGGGSDIVLSQSPAIMSASPGEKVTISCSASSSVSYMYWYQQKPGSSPKPWIYRTSNLASGVPARFSGSGSGTSYSLTISSMEPEDAATYYCQQYHSYPPTFGAGTKLELK

SEQ ID NO: 575 - protein, 845 aa, synthetic construct
  REGION 1..845: note = CD117_A
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCTGGGGSGGGGSGGGGSQVQLVESGGRLVQAGESLRLSCSASGGTFSSYLMAWFRQVPGKEREIVAFITWHGHTTYYTDSVKGRFAISRDNARNTMYLQMNSLRPEDTAVYYCAADQRQEFSGTYSREYNYWGQGTQVTVSSGGGGSGGGGSGGGGSEVQLVESGGGLVQAGGSLRLSCAASGSSINEMGYYRQAPGKERAPRELVATITSGGSTNYADSVKGRFTISRDNAKNTVYLQMNSLKPEDTAVYYCAALRIMTMGRPYDYWGQGTQVTVSS

SEQ ID NO: 576 - protein, 851 aa, synthetic construct
  REGION 1..851: note = CD117_B
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCTGGGGSGGGGSGGGGSQVQLVESGGRLVQAGESLRLSCSASGGTFSSYLMAWFRQVPGKEREIVAFITWHGHTTYYTDSVKGRFAISRDNARNTMYLQMNSLRPEDTAVYYCAADQRQEFSGTYSREYNYWGQGTQVTVSSGGGGSGGGGSGGGGSQVQLVESGGGLVQAGGSLRLSCAASGRTNSNYAMGWFRQAPGKEREFVSVIGRGLGSTYYADSVKGRFTISRDNAKNTVYLQMNSLKPEDAAVYYCAADLEFVFDEMGWIPGEHAYWGRGTLVTVSS

SEQ ID NO: 577 - protein, 847 aa, synthetic construct
  REGION 1..847: note = CD117_C
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCTGGGGSGGGGSGGGGSEVQLVESGGGLVQAGGSLRLSCAASGSSINEMGYYRQAPGKERAPRELVATITSGGSTNYADSVKGRFTISRDNAKNTVYLQMNSLKPEDTAVYYCAALRIMTMGRPYDYWGQGTQVTVSSGGGGSGGGGSGGGGSQVQLVESGGGLVQAGGSLRLSCAASGRTNSNYAMGWFRQAPGKEREFVSVIGRGLGSTYYADSVKGRFTISRDNAKNTVYLQMNSLKPEDAAVYYCAADLEFVFDEMGWIPGEHAYWGRGTLVTVSS

SEQ ID NO: 578 - protein, 847 aa, synthetic construct
  REGION 1..847: note = CD117_D
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCTGGGGSGGGGSGGGGSQVQLVESGGGLVQAGGSLRLSCAASGRTNSNYAMGWFRQAPGKEREFVSVIGRGLGSTYYADSVKGRFTISRDNAKNTVYLQMNSLKPEDAAVYYCAADLEFVFDEMGWIPGEHAYWGRGTLVTVSSGGGGSGGGGSGGGGSEVQLVESGGGLVQAGGSLRLSCAASGSSINEMGYYRQAPGKERAPRELVATITSGGSTNYADSVKGRFTISRDNAKNTVYLQMNSLKPEDTAVYYCAALRIMTMGRPYDYWGQGTQVTVSS

SEQ ID NO: 579 - protein, 853 aa, synthetic construct
  REGION 1..853: note = CD117_E
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCTGGGGSGGGGSGGGGSQVQLVESGGGLVQAGGSLRLSCAASGRTNSNYAMGWFRQAPGKEREFVSVIGRGLGSTYYADSVKGRFTISRDNAKNTVYLQMNSLKPEDAAVYYCAADLEFVFDEMGWIPGEHAYWGRGTLVTVSSGGGGSGGGGSGGGGSQVQLVESGGGLVQAGGSLRLSCAASGRTNSNYAMGWFRQAPGKEREFVSVIGRGLGSTYYADSVKGRFTISRDNAKNTVYLQMNSLKPEDAAVYYCAADLEFVFDEMGWIPGEHAYWGRGTLVTVSS

SEQ ID NO: 580 - protein, 841 aa, synthetic construct
  REGION 1..841: note = CD117_F
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCTGGGGSGGGGSGGGGSEVQLVESGGGLVQAGGSLRLSCAASGSSINEMGYYRQAPGKERAPRELVATITSGGSTNYADSVKGRFTISRDNAKNTVYLQMNSLKPEDTAVYYCAALRIMTMGRPYDYWGQGTQVTVSSGGGGSGGGGSGGGGSEVQLVESGGGLVQAGGSLRLSCAASGSSINEMGYYRQAPGKERAPRELVATITSGGSTNYADSVKGRFTISRDNAKNTVYLQMNSLKPEDTAVYYCAALRIMTMGRPYDYWGQGTQVTVSS

SEQ ID NO: 581 - protein, 849 aa, synthetic construct
  REGION 1..849: note = CD117_E
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCTGGGGSGGGGSGGGGSQVQLVESGGRLVQAGESLRLSCSASGGTFSSYLMAWFRQVPGKEREIVAFITWHGHTTYYTDSVKGRFAISRDNARNTMYLQMNSLRPEDTAVYYCAADQRQEFSGTYSREYNYWGQGTQVTVSSGGGGSGGGGSGGGGSQVQLVESGGRLVQAGESLRLSCSASGGTFSSYLMAWFRQVPGKEREIVAFITWHGHTTYYTDSVKGRFAISRDNARNTMYLQMNSLRPEDTAVYYCAADQRQEFSGTYSREYNYWGQGTQVTVSS

SEQ ID NO: 582 - protein, 1092 aa, synthetic construct
  REGION 1..1092: note = CD133_A
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCTGGGGSGGGGSGGGGSEIQLQQSGPDLMKPGASVKISCKASGYSFTNYYVHWVKQSLDKSLEWIGYVDPFNGDFNYNQKFKDKATLTVDKSSSTAYMHLSSLTSEDSAVYYCARGGLDWYDTSYWYFDVWGAGTAVTVSSGGGGSGGGGSGGGGSNIVMTQSPKSMSMSLGERVTLSCKASENVDTYVSWYQQKPEQSPKVLIYGASNRYTGVPDRFTGSGSATDFSLTISNVQAEDLADYHCGQSYRYPLTFGAGTKLELKGGGGSGGGGSGGGGSEVQLVESGGGLVQPGGSLRLSCAASGFNIYYYGSMHWVRQAPGKGLEWVASISPYYGSTYYADSVKGRFTISADTSKNTAYLQMNSLRAEDTAVYYCARHASSGYGHYAVYGIDYWGQGTLVTVSSGGGGSGGGGSGGGGSDIQMTQSPSSLSASVGDRVTITCRASQGSSYVAWYQQKPGKAPKLLIYSASYLYSGVPSRFSGSRSGTDFTLTISSLQPEDFATYYCQQGVWSLITFGQGTKVEIK

SEQ ID NO: 583 - protein, 1092 aa, synthetic construct
  REGION 1..1092: note = CD133_B
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCTGGGGSGGGGSGGGGSEVQLVESGGGLVQPGGSLRLSCAASGFNIYYYGSMHWVRQAPGKGLEWVASISPYYGSTYYADSVKGRFTISADTSKNTAYLQMNSLRAEDTAVYYCARHASSGYGHYAVYGIDYWGQGTLVTVSSGGGGSGGGGSGGGGSDIQMTQSPSSLSASVGDRVTITCRASQGSSYVAWYQQKPGKAPKLLIYSASYLYSGVPSRFSGSRSGTDFTLTISSLQPEDFATYYCQQGVWSLITFGQGTKVEIKGGGGSGGGGSGGGGSEIQLQQSGPDLMKPGASVKISCKASGYSFTNYYVHWVKQSLDKSLEWIGYVDPFNGDFNYNQKFKDKATLTVDKSSSTAYMHLSSLTSEDSAVYYCARGGLDWYDTSYWYFDVWGAGTAVTVSSGGGGSGGGGSGGGGSNIVMTQSPKSMSMSLGERVTLSCKASENVDTYVSWYQQKPEQSPKVLIYGASNRYTGVPDRFTGSGSATDFSLTISNVQAEDLADYHCGQSYRYPLTFGAGTKLELK

SEQ ID NO: 584 - protein, 964 aa, synthetic construct
  REGION 1..964: note = CD8_A
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCTGGGGSGGGGSGGGGSQVQLVESGGGLVQAGGSLRLSCAASGRTFSGYVMGWFRQAPGKQRKFVAAISRGGLSTSYADSVKGRFTISRDNAKNTVFLQMNTLKPEDTAVYYCAADRSDLYEITAASNIDSWGQGTLVTVSSGGGGSGGGGSGGGGSQVQLVQSGAEVKKPGASVKVSCKASGYTFTSYYMHWVRQAPGQGLEWMGGFDPEDGETIYAQKFQGRVTMTRDTSTSTVYMELSSLRSEDTAVYYCARDQGWGMDVWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQTYSTPYTFGQGTKLEIKR

SEQ ID NO: 585 - protein, 961 aa, synthetic construct
  REGION 1..961: note = CD8_B
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCTGGGGSGGGGSGGGGSQVQLVQSGAEVKKPGASVKVSCKASGYTFTSYYMHWVRQAPGQGLEWMGGFDPEDGETIYAQKFQGRVTMTRDTSTSTVYMELSSLRSEDTAVYYCARDQGWGMDVWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQTYSTPYTFGQGTKLEIKRGSGGGGSGGGGSQVQLVESGGGLVQAGGSLRLSCAASGRTFSGYVMGWFRQAPGKQRKFVAAISRGGLSTSYADSVKGRFTISRDNAKNTVFLQMNTLKPEDTAVYYCAADRSDLYEITAASNIDSWGQGTLVTVSS

SEQ ID NO: 586 - protein, 847 aa, synthetic construct
  REGION 1..847: note = CD117_H
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCTGGGGSGGGGSGGGGSQVQLVESGGGLVQPGGSLRLSCAASGQTFTDYHHHVAWFRQAPGRERDFVAVISWSTGNTDYADSQRARFTISKDNAKNTVYLQMNNLKFEDTAVYYCAARDYHPRFSYSADDYNYWGQGTLVTVSSGGGGSGGGGSGGGGSEVQLVESGGGLVQAGGSLRLSCAASGSSINEMGYYRQAPGKERAPRELVATITSGGSTNYADSVKGRFTISRDNAKNTVYLQMNSLKPEDTAVYYCAALRIMTMGRPYDYWGQGTQVTVSS

SEQ ID NO: 587 - protein, 853 aa, synthetic construct
  REGION 1..853: note = CD117_I
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCTGGGGSGGGGSGGGGSQVQLVESGGGLVQAGGSLRLSCAASGRTNSNYAMGWFRQAPGKEREFVSVIGRGLGSTYYADSVKGRFTISRDNAKNTVYLQMNSLKPEDAAVYYCAADLEFVFDEMGWIPGEHAYWGRGTLVTVSSGGGGSGGGGSGGGGSQVQLVESGGGLVQPGGSLRLSCAASGQTFTDYHHHVAWFRQAPGRERDFVAVISWSTGNTDYADSQRARFTISKDNAKNTVYLQMNNLKFEDTAVYYCAARDYHPRFSYSADDYNYWGQGTLVTVSS

SEQ ID NO: 588 - protein, 852 aa, synthetic construct
  REGION 1..852: note = CD117_J
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCTGGGGSGGGGSGGGGSQVQLVESGGRLVQAGESLRLSCSASGGTFSSYLMAWFRQVPGKEREIVAFITWHGHTTYYTDSVKGRFAISRDNARNTMYLQMNSLRPEDTAVYYCAADQRQEFSGTYSREYNYWGQGTQVTVSSGGGGSGGGGSGGGGSQVQLVESGGGLVQPGGSLRLSCAASGQTFTDYHHHVAWFRQAPGRERDFVAVISWSTGNTDYADSQRARFTISKDNAKNTVYLQMNNLKFEDTAVYYCAARDYHPRFSYSADDYNYWGQGTLVTVSSA

SEQ ID NO: 589 - protein, 6 aa, synthetic construct
  REGION 1..6: note = IgG2c camelid hinge
AHHSED

SEQ ID NO: 590 - protein, 22 aa, synthetic construct
  REGION 1..22: note = IgG2b camelid hinge
EPKTPKPQPQPQPQPQPNPTTE

SEQ ID NO: 591 - protein, 35 aa, synthetic construct
  REGION 1..35: note = IgG2b camelid hinge
EPKIPQPQPKPQPQPQPQPKPQPKPEPECTCPKCP

SEQ ID NO: 592 - protein, 11 aa, synthetic construct
  REGION 1..11: note = IgG2b camelid hinge
TNEVCKCPKCP

SEQ ID NO: 593 - protein, 127 aa, synthetic construct
  REGION 1..127: note = CD117_b1
EVQLVESGGGLVQAGGSLRLSCKASGRTNSNYAMGWFRQAPGKEREFVTVIGRSIGMTYYADSVKGRFTISRDNAKNTVYLQMNSLKPEDTAVYYCAADLEFVFGETGWIPGDDAYWGQGTQVTVSS

SEQ ID NO: 594 - protein, 127 aa, synthetic construct
  REGION 1..127: note = CD117_b2
QVQLVESGGGLVQAGGSLRLSCAASGRTNSNYAMGWFRQAPGKEREFVSVIGRGLGSTYYADSVKGRFTISRDNAKNTVYLQVNSLKPEDTAVYYCAADLEFVFDEMGWIPGEHAYWGRGTLVTVSS

SEQ ID NO: 595 - protein, 127 aa, synthetic construct
  REGION 1..127: note = CD117_b3
EVQLVESGGGLVQPGGSLRLSCTASGRTNSNYAMGWFRQAPGKEREFVSVIGRGLGSTYYADSVKGRFTISRDNAKNTVYLQMNSLKPEDTAVYYCAADLEFVFDETSWIPGEHAYWGRGTLVTVSS

SEQ ID NO: 596 - protein, 127 aa, synthetic construct
  REGION 1..127: note = CD117_b4
EVQLVESGGGLVQAGGSLRLSCAASGRTNSNYAMGWFRQAPGKEREFVTVIGRSIGMTYYADSVKGRFTISRDNAKNTVYLQMNSLKPEDTAVYYCAADLEFVFDETGWIPGDDAYWGQGTLVTVSS

SEQ ID NO: 597 - protein, 127 aa, synthetic construct
  REGION 1..127: note = CD117_b5
QVQLVESGGGLVQAGGSLRLSCAASGRTNSNYAMGWFRQAPGKEREFVSVIGRGLGSTYYADSVKGRFTISRDNAKNTVYLQMNSLKPEDTAVYYCAADLEFVFDGMGWIPGEHAYWGRGTLVTVSS

SEQ ID NO: 598 - protein, 127 aa, synthetic construct
  REGION 1..127: note = CD117_b6
EVQLVESGGGLVQAGGSLRLSCAASGRTNSNYAMGWFRQAPGKEREFVSVIGRGLGSTYYADSVKGRFTISRDNAKNTVYLQMNSLKPEDTAVYYCAADLEFVFDEMGWIPGEHAYWGRGTLVTVSS

SEQ ID NO: 599 - protein, 127 aa, synthetic construct
  REGION 1..127: note = CD117_b7
EVQLVESGGRLVRAGGSLRLSCAASGRTNSNYAMGWFRQAPGKEREFVSVIGRGLGSTYYADSVKGRFTISRDNAKNTVYLQMNSLKPEDTAVYFCAADLEFVFDEMGWIPGEHAYWGRGTLVTVSS

SEQ ID NO: 600 - protein, 127 aa, synthetic construct
  REGION 1..127: note = CD117_b8
EVQLVESGGGLVQAGGSLRLSCAASGRTNSNYAMGWFRQAPGKEREFVSVIGRGLGSTYYADSVKGRFTISRDNAKNTVYLQMNSLKPEDTSVYYCAADLEFVFDEMGWIPGEHAYWGRGTQVTVSS

SEQ ID NO: 601 - protein, 127 aa, synthetic construct
  REGION 1..127: note = CD117_b9
QVQLVESGGGLVQAGGSLGLSCAASGRTFSRMMMGWFRQAPGKEREFVSVIGRGLGSTYYADSVKGRFTISRDNAKNTVYLQMNSLKPEDTAVYYCAADLEFVFDEMGWIPGEHAYWGRGTQVTVSS

SEQ ID NO: 602 - protein, 127 aa, synthetic construct
  REGION 1..127: note = CD117_b10
QVQLVESGGGLVQAGGSLRLSCAASGRTNSNYAMGWFRQAPGKEREFVSVIGRGLGSTYYADSVKGRFTISRDNAKNTVYLQMNSLKPEDTAVYYCAADLEFVFDEMGWIPGEHAYWGRGTQVTVSS

SEQ ID NO: 603 - protein, 127 aa, synthetic construct
  REGION 1..127: note = CD117_b11
QVQLVESGGGLVQAGGSLRLSCAASGRTNSNYAMGWFRQAPGKEREFVSVIGRGLGSTYCADSVKGRFTTSRDNAKNTVYLQMNSLKPEDTAVYYCAADLEFVFDEMGWIPGEHAYWGRGTQVTVSS

SEQ ID NO: 604 - protein, 127 aa, synthetic construct
  REGION 1..127: note = CD117_b13
EVQLVESGGGLVQAGGSLRLSCAASGRTNSNYAMGWFRQAPGKEREFVSVIGRGLGSTYYADSVKGRFTISRDNAKNTVYLQMNSLKPEDTAVYYCAADLEFVFDEMGWIPGEHAYWGRGTQVTVSS

SEQ ID NO: 605 - protein, 127 aa, synthetic construct
  REGION 1..127: note = CD117_b14
EVQLVESGGGLVQAGGSLRLSCAASGRTNSDYAMGWFRQAPGKEREFVSVIGRGLGSTYYADSVKGRFTISRDNAKNTVYLQMNSLKPEDTAVYYCAADLEFVFDEMGWIPGEHAYWGRGTQVTVSS

SEQ ID NO: 606 - protein, 127 aa, synthetic construct
  REGION 1..127: note = CD117_b15
EVQLVESGGGLVQAGGSLRLSCAASGRTNSNYAMGWFRQAPGKEREFVSVIGRGLGSTYYADSVKGRFTISRDNAKNTVYLQMNSLKPEDTAVYYCVADLEFVFDEMGWIPGEHAYWGRGTLVTVSS

SEQ ID NO: 607 - protein, 122 aa, synthetic construct
  REGION 1..122: note = CD117_b16
EVQLVESGGGLAQAGDSLRLSCAASGRFDTSYAMGWFRQAPGKEREFVAGILSGTVKAYEDSVRGRFSISRDKAKNTVYLQMNSLKSEDTAVYYCVADVSFGDLTNGRSDYWGQGTQVTVSS

SEQ ID NO: 608 - protein, 122 aa, synthetic construct
  REGION 1..122: note = CD117_b17
EVQLVESGGGLAQAGDSLRLSCAASGRFDTSYAMGWFRQAPGKEREFVAGILSGTVKAYEDSVRGRFSISRDKAKNTVYLQMNSLKPEDTAVYYCVADVSFGDLTNGRSDYWGQGTQVTVSS

SEQ ID NO: 609 - protein, 122 aa, synthetic construct
  REGION 1..122: note = CD117_b18
EVQLVESGGGLAQAGDSLRLSCAASGRFDTSYAMGWFRQAPGKEREFVAGILSGTVKAYEDSVRGRFSISRDKAKNTVYLQMNSLKPEDTAVYYCVADVSFGDLTNGRSDYWGQGTLVTVSS

SEQ ID NO: 610 - protein, 122 aa, synthetic construct
  REGION 1..122: note = CD117_b19
QVQLVESGGGLAQAGDSLRLSCAASGRFDTSYAMGWFRQAPGKEREFVAGILSGTVKAYEDSVRGRFSISRDKAKNTVYLQMNSLKSEDTAVYYCVADVSFGDLTNGRSDYWGQGTQVTVSS

SEQ ID NO: 611 - protein, 122 aa, synthetic construct
  REGION 1..122: note = CD117_b20
EVQLVESGGGLAQAGDSLRLSCAASGRFDTSYAMGWFRQAPGKEREFVAGILSGTVKAYEDSVRGRFSISRDKAKNTVYLQMNSLKSEDTAVYYCVADVSFGDLTNGRSDYWGQGTLVTVSS

SEQ ID NO: 612 - protein, 122 aa, synthetic construct
  REGION 1..122: note = CD117_b21
EVQLVESGGGLAQAGDSLRLSCAASGRFDTSYAMGWFRQAPGKEREFVAGILSGAVKAYEDSVRGRFSISRDKAKNTVYLQMNSLKPEDTAVYYCVADVSFGDLTNGRSDYWGQGTQVTVSS

SEQ ID NO: 613 - protein, 122 aa, synthetic construct
  REGION 1..122: note = CD117_b22
QVQLVESGGGLAQAGDSLRLSCAASGRFDTSYAMGWFRQAPGKEREFVAGILSGTVKAYEDSVRGRFSISRDKAKNTVYLQMNSLKPEDTAVYYCVADVSFGDLTNGRSDYWGQGTQVTVSS

SEQ ID NO: 614 - protein, 122 aa, synthetic construct
  REGION 1..122: note = CD117_b23
EVQLVESGGGLAQAGDSLRLSCAASGRFDASYAMGWFRQAPGKEREFVAGILSGTVKAYEDSVRGRFSISRDKAKNTVYLQMNSLKSEDTAVYYCVADVSFGDLTNGRSDYWGQGTLVTVSS

SEQ ID NO: 615 - protein, 122 aa, synthetic construct
  REGION 1..122: note = CD117_b24
EVQLVESGGGLAQAGDSLRLSCAASGRFDTSYAMGWFRQAPGKEREFVAGILSGTVKAYEDSVRGRFSISRDQAKNTVYLQMNSLKPEDTAVYYCVADVSFGDLTNGRSDYWGQGTQVTVSS

SEQ ID NO: 616 - protein, 122 aa, synthetic construct
  REGION 1..122: note = CD117_b25
EVQLVESGGGLAQAGDSLRLSCAASGRFDTSYAMGWFRQVPGKERGFVAGILSGTVKAYEDSVRGRFSISRDKAKNTVYLRMNSLKPEDTAVYYCVADVSFGDLTNGRSDYWGQGTQVTVSS

SEQ ID NO: 617 - protein, 126 aa, synthetic construct
  REGION 1..126: note = CD117_b26
EVQLVESGGGLVRAGDSLRLSCAASEGRTFTMRSVGWFRQAPGKEREFVAASNWWSSGSTMYAEYVKGRFTISRDNAKNTVYLQMNSLKPEDTATYYCAASSTVYMSSRLSEYAYWGQGTQVTVSS

SEQ ID NO: 618 - protein, 126 aa, synthetic construct
  REGION 1..126: note = CD117_b27
QVQLVESGGGLVRAGDSLRLSCAASEGRTFTMRSVGWFRQAPGKEREFVAASNWWSSGSTMYAEYVKGRFTISRDNAKNTVYLQMNSLKPEDTATYYCAASSTVYMSSRLSEYAYWGQGTQVTVSS

SEQ ID NO: 619 - protein, 124 aa, synthetic construct
  REGION 1..124: note = CD117_b28
EVQLVESGGGLVQTGGSLTLSCAASKRTLNTYAMGWFRQPPGKRREFVAADSWSGGSTMYAEYVKGRFTISRDNAKNTVYLQMNSLKPEDTATYYCAASSTVYMSSRLSEYAYWGQGTQVTVSS

SEQ ID NO: 620 - protein, 124 aa, synthetic construct
  REGION 1..124: note = CD117_b29
EVQLVESGGGWVQTDDSLRLSCAASGGAFGNYAMGWLRQAPGKGREFVAGIDWSGGSTMYAEYVKGRFTISRDNAKNTVYLQMNSLKPEDTATYYCAASSTVYMSSRLSEYAYWGQGTLVTVSS

SEQ ID NO: 621 - protein, 124 aa, synthetic construct
  REGION 1..124: note = CD117_b30
QVQLVESGGGWVQTDDSLRLSCAASGGAFGNYAMGWLRQAPGKGREFVAGIDWSGGSTMYAEYVKGRFTISRDNAKNTVYLQMNSLKPEDTATYYCAASSTVYMSSRLSEYAYWGQGTQVTVSS

SEQ ID NO: 622 - protein, 124 aa, synthetic construct
  REGION 1..124: note = CD117_b31
QVQLVESGGGSVQTGDSLRLSCVASGSTFGSYAMAWYRQVPGKEREFVAAVRWSGGSTMYAEYVKGRFTISRDNAKNTVYLQMNGLKPEDTATYYCAASSTVYMSSRLSEYAYWGQGTLVTVSS

SEQ ID NO: 623 - protein, 125 aa, synthetic construct
  REGION 1..125: note = CD117_b32
EVQLVESGGRLVQAGESLRLSCSASGGTFSSYLMAWFRQVPGKEREIVAFITWHGHTTYYTDSVKGRFAISRDNARNTMYLQMNSLRPEDTAVYYCAADQRQEFSGTYSREYNYWGQGTLVTVSS

SEQ ID NO: 624 - protein, 125 aa, synthetic construct
  REGION 1..125: note = CD117_b33
QVQLVESGGRLVQAGESLRLSCSASGGTFSSYLMAWFRQVPGKEREIVAFITWHGHTTYYTDSVKGRFAISRDNARNTMYLQMNSLRPEDTAVYYCAADQRQEFSGTYSREYNYWGQGTLVTVSS

SEQ ID NO: 625 - protein, 125 aa, synthetic construct
  REGION 1..125: note = CD117_b35
EVQLVESGGGLVQAGGSLGLACVASGDTLSTYFMGWFRQAPGKEREFVAAIGWSGWLTYYADSMKGRFTISRDNGKNSVYLQMNNLKRDDTAVYYCAAGKTFAAAPSTTLIGDYWGQGTLVTVSS

SEQ ID NO: 626 - protein, 125 aa, synthetic construct
  REGION 1..125: note = CD117_b36
EVQLVESGGGLVQAGGSLRLACVASGDTLSTYFMGWFRQAPGKEREFVAAIGWSGWLTYYADSMKGRFTISRDNGKNSVYLQMNNLKRDDTAVYYCAAGKTFAAAPSTTLIGDYWGQGTQVTVSS

SEQ ID NO: 627 - protein, 125 aa, synthetic construct
  REGION 1..125: note = CD117_b37
QVQLVESGGGLVQAGGSLRLSCVASGDTLSTYFMGWFRQAPGKEREFVAAIGWSGWLTYYADSMKGRFTISRDNGKNSVYLQMNNLKRDDTAVYYCAAGKTFAAAPSTTLIGDYWGQGTLVTVSS

SEQ ID NO: 628 - protein, 126 aa, synthetic construct
  REGION 1..126: note = CD117_b38
EVQLVESGGGLVQVGGSLRLSCAASGRTTGRYFMGWFREAPGKEREFVSAIGWSVGSTYYVDSVKGRFTISRDNAKNTVYLQMNSLKPEDTAVYYCAVGFEFSQGAGTAELIGDYWGQGTQVTVSS

SEQ ID NO: 629 - protein, 126 aa, synthetic construct
  REGION 1..126: note = CD117_b39
EVQLVESGGGLVQVGGSLRLSCAASGRTTGRYFMGWFREAPGKEREFVSAIGWSVGSTYYVDSVKGRFTISRDNANNTVYLQMNSLKPEDTAVYYCAVGFEFSQGAGTAELIGDYWGQGTQVTVSS

SEQ ID NO: 630 - protein, 126 aa, synthetic construct
  REGION 1..126: note = CD117_b40
QVQLVESGGGLVQAGGSLTLSCAAPGRTFSNYFMGWFRQAPGKERDFVAAISWSGFLTYYADSVKGRFTISRDNAKNTVDLQMNRLKPEDTAVYYCAMGSEFSPGAGTAELIGDYWGQGTLVTVSS

SEQ ID NO: 631 - protein, 127 aa, synthetic construct
  REGION 1..127: note = CD117_b42
QVQLVESGGGLVQPGGSLRLSCAASGQTFTDYHHHVAWFRQAPGRERDFVAVISWSTGNTDYADSQRARFTISKDNAKNTAYLQMNNLKFEDTAVYYCAARDYHPRFSYSADDYNYWGQGTQVTVSS

SEQ ID NO: 632 - protein, 131 aa, synthetic construct
  REGION 1..131: note = CD117_b43
EVQLVESGGGLVQAGDSLRLSCTASISTSGNYATFKNYAMGWFRQAPGKAREFVAGISNSGGTTDYADSVKGRFTISRDNARKTTYLQMNRLGPEDTAVYSCAADAWFGPPKRDPTTYSFRGPGTLVTVSS

SEQ ID NO: 633 - protein, 124 aa, synthetic construct
  REGION 1..124: note = CD117_b44
EVQLVESGGGLVQAGDSLRLSCAVSGRTFSSVAMAWLRQGPGKAREFVASISWSGSTTDYADSVKGRFTISRDNAKKMVYLQMNSLKPEDTAVYYCAAGRVPGSSPTIHNYVTWGQGTQVTVSS

SEQ ID NO: 634 - protein, 132 aa, synthetic construct
  REGION 1..132: note = CD117_b45
EVQLVESGGGSAHTGGPLRLSCVGTGPTTGHTFRGYAVAWFRQAPGKEREFVAAVRWNEGQDVYYADSVKGRFTISRDNDKNTVYLQMNSLKPEDTAVYYCAAGVKWGSITFMASSSRYDFWGQGTQVTVSS

SEQ ID NO: 635 - protein, 119 aa, synthetic construct
  REGION 1..119: note = CD117_b47
EVQLVESGGGLVQAGGSLRLSCAASGRTFSYYIMGWFRQAPGKEREFVAAINRSGGTTYYADSVEGRFFISRDNAKNTMYLQMNSLKPEDTAVYYCAAQPDSRGTYGYWGQGTLVTVSS

SEQ ID NO: 636 - protein, 125 aa, synthetic construct
  REGION 1..125: note = CD117_b48
EVQLVESGGGLVQAGGSLRLSCIDSGRTFSRYYMGWFRQAPGKEREFVAAISQNGYTKWYAESVKGRSAISRDIAKNTVDLQMNSLKPEDTAVYYCAATSILEFSRDTASDFEYYGQGTLVTVSS

SEQ ID NO: 637 - protein, 122 aa, synthetic construct
  REGION 1..122: note = CD117_b49
EVQLVESGGGLVRAGDSLRLSCSASGRTFSKWTMAWFRQAPGKEREFVAGMSGDATYYAASVKGRFTSSRDNSKNTVYLQMDSLKPEDTAVYFCAIYYGSGSYFQQNSYDTWGQGTQVTVSS

SEQ ID NO: 638 - protein, 123 aa, synthetic construct
  REGION 1..123: note = CD117_b50
QVQLVESGGGLVQAGGSLRLSCTASGRTESTYFMSWFRQTPGKERDFVAVINYSGDVTTYADSVKGRFTISRDNAKNTVHLQMNSLTVEDTAVYYCAVHRTGPMNRADSYDYWGQGTLVTVSS

SEQ ID NO: 639 - protein, 125 aa, synthetic construct
  REGION 1..125: note = CD117_b51
EVQLVESGGGLVQAGGSLKLSCAASGNIANTYRMAWYGQAPGKQRELVANITSDGIASYADSVKGRFTISRDNAKNTVYLQMNSLKAEDTAVYYCNIVYRRCSRGGYGCYSEYSWGQGTLVTVSS

SEQ ID NO: 640 - protein, 569 aa, synthetic construct
  REGION 1..569: note = NivG.690
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 641 - protein, 569 aa, synthetic construct
  REGION 1..569: note = NivG.691
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 642 - protein, 569 aa, synthetic construct
  REGION 1..569: note = NivG.693
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 643 - protein, 569 aa, synthetic construct
  REGION 1..569: note = NivG.694
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 644 - protein, 569 aa, synthetic construct
  REGION 1..569: note = NivG.695
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 645 - protein, 569 aa, synthetic construct
  REGION 1..569: note = NivG.696
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 646 - protein, 569 aa, synthetic construct
  REGION 1..569: note = NivG.697
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 647 - protein, 569 aa, synthetic construct
  REGION 1..569: note = NivG.699
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 648 - protein, 569 aa, synthetic construct
  REGION 1..569: note = NivG.700
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 649 - protein, 569 aa, synthetic construct
  REGION 1..569: note = NivG.701
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 650 - protein, 569 aa, synthetic construct
  REGION 1..569: note = NivG.702
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 651 - protein, 569 aa, synthetic construct
  REGION 1..569: note = NivG.740
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 652 - protein, 569 aa, synthetic construct
  REGION 1..569: note = NivG.742
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 653 - protein, 569 aa, synthetic construct
  REGION 1..569: note = NivG.743
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 654 - protein, 569 aa, synthetic construct
  REGION 1..569: note = NivG.744
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 655 - protein, 569 aa, synthetic construct
  REGION 1..569: note = NivG.748
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 656 - protein, 569 aa, synthetic construct
  REGION 1..569: note = NivG.749
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 657 - protein, 569 aa, synthetic construct
  REGION 1..569: note = NivG.750
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 658 - protein, 569 aa, synthetic construct
  REGION 1..569: note = NivG.752
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 659 - protein, 569 aa, synthetic construct
  REGION 1..569: note = NivG.753
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 660 - protein, 569 aa, synthetic construct
  REGION 1..569: note = NivG.754
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 661 - protein, 569 aa, synthetic construct
  REGION 1..569: note = NivG.755
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 662 - protein, 569 aa, synthetic construct
  REGION 1..569: note = NivG.756
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 663 - protein, 569 aa, synthetic construct
  REGION 1..569: note = NivG.757
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 664 - protein, 569 aa, synthetic construct
  REGION 1..569: note = NivG.758
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 665 - protein, 569 aa, synthetic construct
  REGION 1..569: note = NivG.759
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 666 - protein, 569 aa, synthetic construct
  REGION 1..569: note = NivG.760
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 667 - protein, 569 aa, synthetic construct
  REGION 1..569: note = NivG.761
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 668 - protein, 569 aa, synthetic construct
  REGION 1..569: note = NivG.762
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 669 - protein, 569 aa, synthetic construct
  REGION 1..569: note = NivG.763
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 670 - protein, 569 aa, synthetic construct
  REGION 1..569: note = NivG.764
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 671 - protein, 569 aa, synthetic construct
  REGION 1..569: note = NivG.765
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 672 - protein, 569 aa, synthetic construct
  REGION 1..569: note = NivG.766
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 673 - protein, 569 aa, synthetic construct
  REGION 1..569: note = NivG.767
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 674 - protein, 569 aa, synthetic construct
  REGION 1..569: note = NivG.768
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 675 - protein, 569 aa, synthetic construct
  REGION 1..569: note = NivG.769
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 676 - protein, 569 aa, synthetic construct
  REGION 1..569: note = NivG.770
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 677 - protein, 569 aa, synthetic construct
  REGION 1..569: note = NivG.771
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 678 - protein, 569 aa, synthetic construct
  REGION 1..569: note = NivG.772
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 679 - protein, 569 aa, synthetic construct
  REGION 1..569: note = NivG.773
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 680 - protein, 569 aa, synthetic construct
  REGION 1..569: note = NivG.774
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 681 - protein, 569 aa, synthetic construct
  REGION 1..569: note = NivG.775
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 682 - protein, 569 aa, synthetic construct
  REGION 1..569: note = NivG.776
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 683 - protein, 569 aa, synthetic construct
  REGION 1..569: note = NivG.777
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 684 - protein, 569 aa, synthetic construct
  REGION 1..569: note = NivG.778
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 685 - protein, 569 aa, synthetic construct
  REGION 1..569: note = NivG.779
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 686 - protein, 569 aa, synthetic construct
  REGION 1..569: note = NivG.780
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 687 - protein, 569 aa, synthetic construct
  REGION 1..569: note = NivG.781
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 688 - protein, 569 aa, synthetic construct
  REGION 1..569: note = NivG.782
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 689 - protein, 569 aa, synthetic construct
  REGION 1..569: note = NivG.783
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 690 - protein, 569 aa, synthetic construct
  REGION 1..569: note = NivG.784
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 691 - protein, 569 aa, synthetic construct
  REGION 1..569: note = NivG.785
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 692 - protein, 569 aa, synthetic construct
  REGION 1..569: note = NivG.786
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 693 - protein, 569 aa, synthetic construct
  REGION 1..569: note = NivG.787
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 694 - protein, 569 aa, synthetic construct
  REGION 1..569: note = NivG.788
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 695 - protein, 569 aa, synthetic construct
  REGION 1..569: note = NivG.789
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 696 - protein, 569 aa, synthetic construct
  REGION 1..569: note = NivG.790
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 697 - protein, 569 aa, synthetic construct
  REGION 1..569: note = NivG.791
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 698 - protein, 569 aa, synthetic construct
  REGION 1..569: note = NivG.792
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 699 - protein, 569 aa, synthetic construct
  REGION 1..569: note = NivG.793
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 700 - protein, 569 aa, synthetic construct
  REGION 1..569: note = NivG.794
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 701 - protein, 569 aa, synthetic construct
  REGION 1..569: note = NivG.795
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 702 - protein, 569 aa, synthetic construct
  REGION 1..569: note = NivG.796
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 703 - protein, 569 aa, synthetic construct
  REGION 1..569: note = NivG.797
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 704 - protein, 569 aa, synthetic construct
  REGION 1..569: note = NivG.798
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 705 - protein, 569 aa, synthetic construct
  REGION 1..569: note = NivG.799
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 706 - protein, 569 aa, synthetic construct
  REGION 1..569: note = NivG.800
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 707 - protein, 569 aa, synthetic construct
  REGION 1..569: note = NivG.801
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 708 - protein, 569 aa, synthetic construct
  REGION 1..569: note = NivG.802
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 709 - protein, 569 aa, synthetic construct
  REGION 1..569: note = NivG.803
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 710 - protein, 569 aa, synthetic construct
  REGION 1..569: note = NivG.804
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 711 - protein, 569 aa, synthetic construct
  REGION 1..569: note = NivG.805
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 712 - protein, 569 aa, synthetic construct
  REGION 1..569: note = NivG.806
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 713 - protein, 569 aa, synthetic construct
  REGION 1..569: note = NivG.807
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 714 - protein, 569 aa, synthetic construct
  REGION 1..569: note = NivG.808
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 715 - protein, 569 aa, synthetic construct
  REGION 1..569: note = NivG.809
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 716 - protein, 569 aa, synthetic construct
  REGION 1..569: note = NivG.810
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 717 - protein, 569 aa, synthetic construct
  REGION 1..569: note = NivG.811
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 718 - protein, 569 aa, synthetic construct
  REGION 1..569: note = NivG.812
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 719 - protein, 569 aa, synthetic construct
  REGION 1..569: note = NivG.813
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 720 - protein, 569 aa, synthetic construct
  REGION 1..569: note = NivG.814
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 721 - protein, 569 aa, synthetic construct
  REGION 1..569: note = NivG.815
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 722 - protein, 569 aa, synthetic construct
  REGION 1..569: note = NivG.816
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 723 - protein, 569 aa, synthetic construct
  REGION 1..569: note = NivG.817
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 724 - protein, 569 aa, synthetic construct
  REGION 1..569: note = NivG.818
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 725 - protein, 569 aa, synthetic construct
  REGION 1..569: note = NivG.819
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 726 - protein, 569 aa, synthetic construct
  REGION 1..569: note = NivG.820
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 727 - protein, 569 aa, synthetic construct
  REGION 1..569: note = NivG.821
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 728 - protein, 569 aa, synthetic construct
  REGION 1..569: note = NivG.822
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 729 - protein, 569 aa, synthetic construct
  REGION 1..569: note = NivG.823
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 730 - protein, 569 aa, synthetic construct
  REGION 1..569: note = NivG.824
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 731 - protein, 569 aa, synthetic construct
  REGION 1..569: note = NivG.825
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 732 - protein, 569 aa, synthetic construct
  REGION 1..569: note = NivG.826
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 733 - protein, 569 aa, synthetic construct
  REGION 1..569: note = NivG.827
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 734 - protein, 569 aa, synthetic construct
  REGION 1..569: note = NivG.828
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 735 - protein, 569 aa, synthetic construct
  REGION 1..569: note = NivG.829
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 736 - protein, 569 aa, synthetic construct
  REGION 1..569: note = NivG.830
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 737 - protein, 569 aa, synthetic construct
  REGION 1..569: note = NivG.831
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 738 - protein, 569 aa, synthetic construct
  REGION 1..569: note = NivG.832
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 739 - protein, 569 aa, synthetic construct
  REGION 1..569: note = NivG.833
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 740 - protein, 569 aa, synthetic construct
  REGION 1..569: note = NivG.834
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 741 - protein, 569 aa, synthetic construct
  REGION 1..569: note = NivG.835
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 742 - protein, 569 aa, synthetic construct
  REGION 1..569: note = NivG.836
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 743 - protein, 569 aa, synthetic construct
  REGION 1..569: note = NivG.837
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 744 - protein, 569 aa, synthetic construct
  REGION 1..569: note = NivG.838
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 745 - protein, 569 aa, synthetic construct
  REGION 1..569: note = NivG.839
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 746 - protein, 569 aa, synthetic construct
  REGION 1..569: note = NivG.840
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 747 - protein, 569 aa, synthetic construct
  REGION 1..569: note = NivG.841
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 748 - protein, 569 aa, synthetic construct
  REGION 1..569: note = NivG.842
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 749 - protein, 569 aa, synthetic construct
  REGION 1..569: note = NivG.843
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 750 - protein, 569 aa, synthetic construct
  REGION 1..569: note = NivG.844
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 751 - protein, 569 aa, synthetic construct
  REGION 1..569: note = NivG.845
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 752 - protein, 569 aa, synthetic construct
  REGION 1..569: note = NivG.846
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 753 - protein, 569 aa, synthetic construct
  REGION 1..569: note = NivG.847
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 754 - protein, 569 aa, synthetic construct
  REGION 1..569: note = NivG.848
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 755 - protein, 569 aa, synthetic construct
  REGION 1..569: note = NivG.849
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 756 - protein, 569 aa, synthetic construct
  REGION 1..569: note = NivG.850
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 757 - protein, 569 aa, synthetic construct
  REGION 1..569: note = NivG.851
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 758 - protein, 569 aa, synthetic construct
  REGION 1..569: note = NivG.852
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 759 - protein, 569 aa, synthetic construct
  REGION 1..569: note = NivG.853
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 760 - protein, 569 aa, synthetic construct
  REGION 1..569: note = NivG.854
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 761 - protein, 569 aa, synthetic construct
  REGION 1..569: note = NivG.855
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 762 - protein, 569 aa, synthetic construct
  REGION 1..569: note = NivG.856
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 763 - protein, 569 aa, synthetic construct
  REGION 1..569: note = NivG.857
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 764 - protein, 569 aa, synthetic construct
  REGION 1..569: note = NivG.858
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 765 - protein, 569 aa, synthetic construct
  REGION 1..569: note = NivG.859
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSQATAANPVFTVFKDNEILYRAQLASEDTNAQKTITNCFLLKNKIWCISLVEIYDTGDNVIRPKLFAVKIPEQCT

SEQ ID NO: 766 - protein, 569 aa, synthetic construct
  REGION 1..569: note = NivG.860
MKKINEGLLDSKILSAFNTVIALLGSIVIIVMNIMIIQQYTRSTDNQAVIKDALQGIQQQIKGLADKIGTEIGPKVSLIDTSSTITIPANIGLLGSKISQSTASINENVNEKCKFTLPPLKIHECQISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILQSTYWSGSLMMTRLAVKPKSNGGGYNQHQLALRSIEKGRYDKVMPYGPSGIKQGDTLYFPAVGFLVRTEFKYQDSNCPITKCQYSKPENCRLSMGIRPNSHYILRSGLLKYQLSDGENPKVVFIEISDQRLSIGSPSKIYDSLGQPVFYQASFSWDTMIKFGDVLTVNPLVVNWRQNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 767 - protein, 562 aa, synthetic construct
  REGION 1..562: note = BaEVTR
MGFTTKIIFLYNLVLVYAGFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLVQFVKDRISVVQALVLTQQYHQLKPLEYEP

SEQ ID NO: 768 - protein, 2008 aa, synthetic construct
  REGION 1..2008: note = MLV-gag
MGQTVTTPLSLTLGHWKDVERIAHNQSVDVKKRRWVTFCSAEWPTFNVGWPRDGTFNRDLITQVKIKVFSPGPHGHPDQVPYIVTWEALAFDPPPWVKPFVHPKPPPPLPPSAPSLPLEPPRSTPPRSSLYPALTPSLGAKPKPQVLSDSGGPLIDLLTEDPPPYRDPRPPPSDRDGNGGEATPAGEAPDPSPMASRLRGRREPPVADSTTSQAFPLRAGGNGQLQYWPFSSSDLYNWKNNNPSFSEDPGKLTALIESVLITHQPTWDDCQQLLGTLLTGEEKQRVLLEARKAVRGDDGRPTQLPNEVDAAFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLERLEDLKNKTLGDLVREAEKIFNKRETPEEREERIRRETEEKEERRRTEDEQKEKERDRRRHREMSKLLATVVSGQKQDRQGGERRRSQLDRDQCAYCKEKGHWAKDCPKKPRGPRGPRPQTSLLTLDDSGGSLQLPPLERLTLGSLQLPPLERLTLGSLQLPPLERLTLTSTLLMENSSGDYKDDDDKKRTADGSEFESPKKKRKVSGGSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKV

SEQ ID NO: 769 - protein, 617 aa, synthetic construct
  REGION 1..617: note = Measles H protein
MSPQRDRINAFYKDNPHPKGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPYLFNVPIKEAGEDCHAPTYLPAEVDGDVKLSSNLVILPGQDLQYVLATYDTSRVEHAVVYYVYSPSRSFSYFYPFRLPIKGVPIELQVECFTWDQKLWCRHFCVLADSESGGHITHSGMEGMGVSCTVTREDGTNRR

SEQ ID NO: 770 - protein, 578 aa, synthetic construct
  REGION 1..578: note = Canine distemper virus H protein
MLPYQDKVGAFYKDNARANSTKLSLVTEGHGGRRPPYLLFVLLILLVGILALLAITGVRFHQVSTSNMEFSRLLKEDMEKSEAVHHQVIDVLTPLFKIIGDEIGLRLPQKLNEIKQFILQKTNFFNPNREFDFRDLHWCINPPSTVKVNFTNYCESIGIRKAIASAANPILLSALSGGRGDIFPPHRCSGATTSVGKVFPLSVSLSMSLISRTSEVINMLTAISDGVYGKTYLLVPDDIEREFDTREIRVFEIGFIKRWLNDMPLLQTTNYMVLPKNSKAKVCTIAVGELTLASLCVEESTVLLYHDSSGSQDGILVVTLGIFWATPMDHIEEVIPVAHPSMKKIHITNHRGFIKDSIATWMVPALASEKQEEQKGCLESACQRKTYPMCNQASWEPFGGRQLPSYGRLTLPLDASVDLQLNISFTYGPVILNGDGMDYYESPLLNSGWLTIPPKDGTISGLINKAGRGDQFTVLPHVLTFAPRESSGNCYLPIQTSQIRDRDVLIESNIVVLPTQSIRYVIATYDISRSDHAIVYYVYDPIRTISYTHPFRLTTKGRPDFLRIECFVWDDNLWCHQF